(19) United States
(12) Patent Application Publication
(10) Pub. No.: US 2025/0313524 A1
(43) Pub. Date: Oct. 9, 2025

(54) PI3K-ALPHA INHIBITORS AND METHODS OF USE THEREOF

(71) Applicant: Relay Therapeutics, Inc., Cambridge, MA (US)

(72) Inventors: Yue Pan, Cambridge, MA (US); Alessandro Boezio, Cambridge, MA (US); Cary Griffin Fridrich, Cambridge, MA (US); Elaine B. Krueger, Cambridge, MA (US); Demetri T. Moustakas, Cambridge, MA (US); Kevin David Raynor, Cambridge, MA (US); Kelley C. Shortsleeves, Cambridge, MA (US); Thomas H. McLean, Cambridge, MA (US); Michael Paul DeNinno, Gales Ferry, CT (US); Christopher Thomson, Hoddesdon, Hertfordshire (GB); Alexandre Larivee, Montreal (CA); Andrew J. Burnie, Montreal (CA); Bren-Jordan Atienza, Montreal (CA); Christian Perreault, Montreal (CA); Gaetan Maertens, Montreal (CA); Tarek Mohamed, Montreal (CA); Thomas Lepitre, Montreal (CA); Erich W. Baum, Cambridge, MA (US); Hakan Gunaydin, Cambridge, MA (US)

(21) Appl. No.: 18/863,452

(22) PCT Filed: May 10, 2023
(86) PCT No.: PCT/US2023/021668

§ 371 Date: Nov. 6, 2024

Related U.S. Application Data
(60) Provisional application No. 63/364,459, filed on May 10, 2022.

(51) Int. Cl. C07C 211/29; A61K 31/137; A61K 31/325; A61K 31/403; A61K 31/4184; A61K 31/423; A61P 35/00; C07C 271/06; C07D 209/54; C07D 235/02; C07D 263/52

(52) U.S. Cl. CPC C07C 211/29; A61K 31/137; A61K 31/325; A61K 31/403; A61K 31/4184; A61K 31/423; A61P 35/00; C07C 271/06; C07D 209/54; C07D 235/02; C07D 263/52

(57) ABSTRACT

The present disclosure relates to novel compounds and pharmaceutical compositions thereof, and methods for inhibiting the activity of PI3Kα enzymes with the compounds and compositions of the disclosure. The present disclosure further relates to, but is not limited to, methods for treating disorders associated with PI3Kα signaling with the compounds and compositions of the disclosure.

### CROSS-REFERENCE TO RELATED APPLICATIONS

This application claims the benefit of U.S. Provisional Application No. 63/364,459, filed on May 10, 2022, the entirety of which is hereby incorporated by reference.

### BACKGROUND

Phosphatidylinositol 3-kinases (PI3Ks) comprise a family of lipid kinases that catalyze the transfer of phosphate to the D-3′ position of inositol lipids to produce phosphoinositol-3-phosphate (PIP), phosphoinositol-3,4-diphosphate (PIP₂) and phosphoinositol-3,4,5-triphosphate (PIP₃), which, in turn, act as second messengers in signaling cascades by docking proteins containing pleckstrin-homology, FYVE, Phox and other phospholipid-binding domains into a variety of signaling complexes often at the plasma membrane (Vanhaesebroeck et al., Annu. Rev. Biochem 70:535 (2001); Katso et al., Annu. Rev. Cell Dev. Biol. 17:615 (2001)). Of the two Class 1 PI3K sub-classes, Class 1A PI3Ks are heterodimers composed of a catalytic p110 subunit (alpha, beta, or delta isoforms) constitutively associated with a regulatory subunit that can be p85 alpha, p55 alpha, p50 alpha, p85 beta, or p55 gamma. The Class 1B sub-class has one family member, a heterodimer composed of a catalytic p110 gamma subunit associated with one of two regulatory subunits, p101 or p84 (Fruman et al., Annu Rev. Biochem. 67:481 (1998); Suire et al., Curr. Biol. 15:566 (2005)). The modular domains of the p85/55/50 subunits include Src Homology (SH2) domains that bind phosphotyrosine residues in a specific sequence context on activated receptor and cytoplasmic tyrosine kinases, resulting in activation and localization of Class 1A PI3Ks. Class 1B PI3K is activated directly by G protein-coupled receptors that bind a diverse repertoire of peptide and non-peptide ligands (Stephens et al., Cell 89:105 (1997); Katso et al., Annu. Rev. Cell Dev. Biol. 17:615-675 (2001)).

Consequently, the resultant phospholipid products of Class I PI3Ks link upstream receptors with downstream cellular activities including proliferation, survival, chemotaxis, cellular trafficking, motility, metabolism, inflammatory and allergic responses, transcription and translation (Cantley et al., Cell 64:281 (1991); Escobedo and Williams, Nature 335:85 (1988); Fantl et al., Cell 69:413 (1992)). In many cases, PIP₂ and PIP₃ recruit Aid, the product of the human homologue of the viral oncogene v-Akt, to the plasma membrane where it acts as a nodal point for many intracellular signaling pathways important for growth and survival (Fantl et al., Cell 69:413-423 (1992); Bader et al., Nature Rev. Cancer 5:921 (2005); Vivanco and Sawyer, Nature Rev. Cancer 2:489 (2002)).

Aberrant regulation of PI3K, which often increases survival through Aid activation, is one of the most prevalent events in human cancer and has been shown to occur at multiple levels. The tumor suppressor gene PTEN, which dephosphorylates phosphoinositides at the 3′ position of the inositol ring, and in so doing antagonizes PI3K activity, is functionally deleted in a variety of tumors. In other tumors, the genes for the p110 alpha isoform, PIK3CA, and for Akt are amplified, and increased protein expression of their gene products has been demonstrated in several human cancers. Furthermore, mutations and translocation of p85 alpha that serve to up-regulate the p85-p110 complex have been described in human cancers. Finally, somatic missense mutations in PIK3CA that activate downstream signaling pathways have been described at significant frequencies in a wide diversity of human cancers (Kang et el., Proc. Natl. Acad. Sci. USA 102:802 (2005); Samuels et al., Science 304:554 (2004); Samuels et al., Cancer Cell 7:561-573 (2005)). These observations show that deregulation of phosphoinositol-3 kinase, and the upstream and downstream components of this signaling pathway, is one of the most common deregulations associated with human cancers and proliferative diseases (Parsons et al., Nature 436:792 (2005); Hennessey at el., Nature Rev. Drug Disc. 4:988-1004 (2005)).

In view of the above, inhibitors of PI3Kα would be of particular value in the treatment of proliferative disease and other disorders. While multiple inhibitors of PI3Ks have been developed (for example, taselisib, alpelisib, buparlisib and others), these molecules inhibit multiple Class 1A PI3K isoforms. Inhibitors that are active against multiple Class 1A PI3K isoforms are known as “pan-PI3K” inhibitors. A major hurdle for the clinical development of existing PI3K inhibitors has been the inability to achieve the required level of target inhibition in tumors while avoiding toxicity in cancer patients. Pan-PI3K inhibitors share certain target-related toxicities including diarrhea, rash, fatigue, and hyperglycemia. The toxicity of PI3K inhibitors is dependent on their isoform selectivity profile. Inhibition of PI3Kα is associated with hyperglycemia and rash, whereas inhibition of PI3Kδ or PI3Kγ is associated with diarrhea, myelosuppression, and transaminitis (Hanker et al., Cancer Discovery (2019) PMID: 30837161. Therefore, selective inhibitors of PI3Kα may increase the therapeutic window, enabling sufficient target inhibition in the tumor while avoiding dose-limiting toxicity in cancer patients.

### SUMMARY

In some embodiments, the present disclosure provides a compound of formula I:

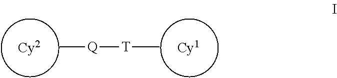

or a pharmaceutically acceptable salt thereof, wherein each of Cy¹, Cy², Q, and T is as defined in embodiments and classes and subclasses herein.

In some embodiments, the present disclosure provides a pharmaceutical composition comprising a compound of formula I, or a pharmaceutically acceptable salt thereof, and a pharmaceutically acceptable carrier, adjuvant, or diluent.

In some embodiments, the present disclosure provides a method of treating a PI3Kα-mediated disorder comprising administering to a patient in need thereof a compound of formula I, or composition comprising said compound.

In some embodiments, the present disclosure provides a process for providing a compound of formula I, or synthetic intermediates thereof.

In some embodiments, the present disclosure provides a process for providing pharmaceutical compositions comprising compounds of formula I.

### DETAILED DESCRIPTION

### 1. General Description of Certain Embodiments of the Disclosure

Compounds of the present disclosure, and pharmaceutical compositions thereof, are useful as inhibitors of PI3Kα. In some embodiments, the present disclosure provides a compound of formula I:

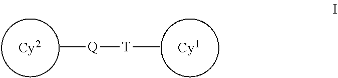

or a pharmaceutically acceptable salt thereof, wherein:
Cy¹ is phenyl; naphthyl; cubanyl; adamantyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; wherein Cy¹ is substituted with n instances of R¹;
Cy² is phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein Cy² is substituted with m instances of R²;
Q is L^{Q};
T is a bivalent C₁₋₃ aliphatic chain substituted with q instances of R^{T};
each R¹ is independently -L¹-R^{1A};
each R² is independently -L²-R^{2A};
each R^{T} is independently -L^{T}-R^{TA}; or
   two instances of R^{T} are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p¹ instances of R^{TTC};
   two instances of R¹ are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p² instances of R^{11C};
   two instances of R² are taken together with their intervening atoms to form a 3-7 membered saturated, partially unsaturated, or aromatic carbocyclic ring or a 3-7 membered saturated or partially unsaturated monocyclic heterocyclic ring having 1-3 heteroatoms independently selected from nitrogen, oxygen, and sulfur; wherein the ring is substituted with p³ instances of R^{22C};
   one instance of R^{T} and one instance of R¹ are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p⁴ instances of R^{TLC}; or
   one instance of R^{T} and one instance of R^{L} are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p⁵ instances of R^{TLC};
each of L¹, L², L^{Q}, and L^{T} is independently a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —N(R)C(NR)—, —N(R)C(NOR)—, —N(R)C(NCN)—, —C(O)N(R)—, —N(R)S(O)₂—, —S(O)₂N(R)—, —O—, —C(O)—, —OC(O)—, —C(O)O—, —S—, —S(O)—, or —S(O)₂—;
each R^{1A} is independently R^{A} or R^{B} substituted by r¹ instances of R^{1C};
each R^{2A} is independently R^{A} or R^{B} substituted by r² instances of R^{2C};
each R^{TA} is independently R^{A} or RB substituted by r³ instances of R^{TC};
each R^{L} is independently R^{A} or R^{B} substituted by r⁴ instances of R^{LC};
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{1C}, R^{2C}, R^{TC}, R^{TTC}, R^{11C}, R^{22C}, R^{T1C}, R^{TLC}, and R^{LC} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of n, m, q, p¹, p², p³, p⁴, p⁵, r¹, r², r³, and r⁴ is independently 0, 1, 2, 3, 4, or 5.

### 2. Compounds and Definitions

Compounds of the present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” or “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “alkyl”, unless otherwise indicated, as used herein, refers to a monovalent aliphatic hydrocarbon radical having a straight chain, branched chain, monocyclic moiety, or polycyclic moiety or combinations thereof, wherein the radical is optionally substituted at one or more carbons of the straight chain, branched chain, monocyclic moiety, or polycyclic moiety or combinations thereof with one or more substituents at each carbon, wherein the one or more substituents are independently C₁-C₁₀ alkyl. Examples of “alkyl” groups include methyl, ethyl, propyl, isopropyl, butyl, iso-butyl, sec-butyl, tert-butyl, pentyl, hexyl, heptyl, cyclopropyl, cyclobutyl, cyclopentyl, cyclohexyl, cycloheptyl, norbornyl, and the like.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR, (as in N-substituted pyrrolidinyl)).

The term “unsaturated,” as used herein, means that a moiety has one or more units of unsaturation.

As used herein, the term “C₁₋₈ (or C₁₋₆, or C₁₋₄) bivalent saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl,” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present disclosure, “aryl” refers to an aromatic ring system which includes, but is not limited to, phenyl, biphenyl, naphthyl, anthracyl and the like, which may bear one or more substituents.

The terms “heteroaryl” or “heteroaromatic”, unless otherwise defined, as used herein refers to a monocyclic aromatic 5-6 membered ring containing one or more heteroatoms, for example one to three heteroatoms, such as nitrogen, oxygen, and sulfur, or an 8-10 membered polycyclic ring system containing one or more heteroatoms, wherein at least one ring in the polycyclic ring system is aromatic, and the point of attachment of the polycyclic ring system is through a ring atom on an aromatic ring. A heteroaryl ring may be linked to adjacent radicals though carbon or nitrogen. Examples of heteroaryl rings include but are not limited to furan, thiophene, pyrrole, thiazole, oxazole, isothiazole, isoxazole, imidazole, pyrazole, triazole, pyridine, pyrimidine, indole, etc. For example, unless otherwise defined, 1,2,3,4-tetrahydroquinoline is a heteroaryl ring if its point of attachment is through the benzo ring, e.g.:

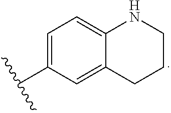 [SMILES: *c1ccc2c(c1)CCCN2]

The terms “heterocyclyl” or “heterocyclic group”, unless otherwise defined, refer to a saturated or partially unsaturated 3-10 membered monocyclic or 7-14 membered polycyclic ring system, including bridged or fused rings, and whose ring system includes one to four heteroatoms, such as nitrogen, oxygen, and sulfur. A heterocyclyl ring may be linked to adjacent radicals through carbon or nitrogen.

The term “partially unsaturated” in the context of rings, unless otherwise defined, refers to a monocyclic ring, or a component ring within a polycyclic (e.g., bicyclic, tricyclic, etc.) ring system, wherein the component ring contains at least one degree of unsaturation in addition to those provided by the ring itself, but is not aromatic. Examples of partially unsaturated rings include, but are not limited to, 3,4-dihydro-2H-pyran, 3-pyrroline, 2-thiazoline, etc. Where a partially unsaturated ring is part of a polycyclic ring system, the other component rings in the polycyclic ring system may be saturated, partially unsaturated, or aromatic, but the point of attachment of the polycyclic ring system is on a partially unsaturated component ring. For example, unless otherwise defined, 1,2,3,4-tetrahydroquinoline is a partially unsaturated ring if its point of attachment is through the piperidino ring, e.g.:

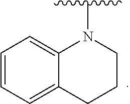 [SMILES: *N1CCCc2ccccc21]

The term “saturated” in the context of rings, unless otherwise defined, refers to a 3-10 membered monocyclic ring, or a 7-14 membered polycyclic (e.g., bicyclic, tricyclic, etc.) ring system, wherein the monocyclic ring or the component ring that is the point of attachment for the polycyclic ring system contains no additional degrees of unsaturation in addition to that provided by the ring itself. Examples of monocyclic saturated rings include, but are not limited to, azetidine, oxetane, cyclohexane, etc. Where a saturated ring is part of a polycyclic ring system, the other component rings in the polycyclic ring system may be saturated, partially unsaturated, or aromatic, but the point of attachment of the polycyclic ring system is on a saturated component ring. For example, unless otherwise defined, 2-azaspiro[3.4]oct-6-ene is a saturated ring if its point of attachment is through the azetidino ring, e.g.:

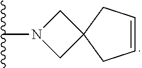 [SMILES: *N1CC2(CC=CC2)C1]

The terms “alkylene”, “arylene”, “cycloalkylene”, “heteroarylene”, “heterocycloalkylene”, and the other similar terms with the suffix “-ylene” as used herein refers to a divalently bonded version of the group that the suffix modifies. For example, “alkylene” is a divalent alkyl group connecting the groups to which it is attached.

As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

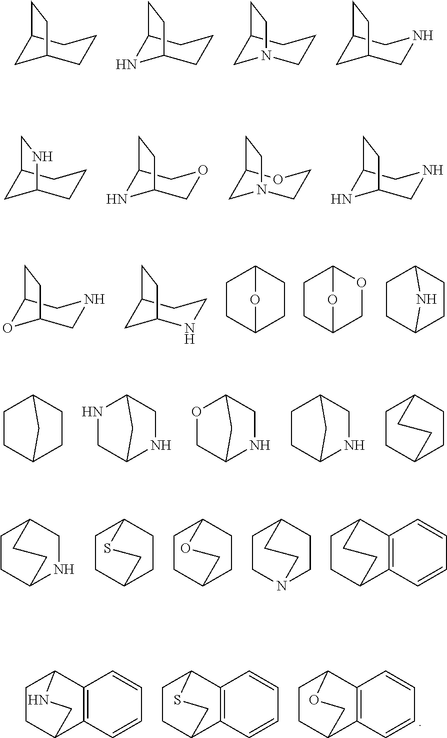

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)(OR^{∘})R^{∘}; —P(O)R^{∘}₂; —OP(O)R^{∘}₂; —OP(O)(OR^{∘})₂; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “isomer” as used herein refers to a compound having the identical chemical formula but different structural or optical configurations. The term “stereoisomer” as used herein refers to and includes isomeric molecules that have the same molecular formula but differ in positioning of atoms and/or functional groups in the space. All stereoisomers of the present compounds (e.g., those which may exist due to asymmetric carbons on various substituents), including enantiomeric forms and diastereomeric forms, are contemplated within the scope of this disclosure. Therefore, unless otherwise stated, single stereochemical isomers as well as mixtures of enantiomeric, diastereomeric, and geometric (or conformational) isomers of the present compounds are within the scope of the disclosure.

The term “tautomer” as used herein refers to one of two or more structural isomers which exist in equilibrium and which are readily converted from one isomeric form to another. It is understood that tautomers encompass valence tautomers and proton tautomers (also known as prototropic tautomers). Valence tautomers include interconversions by reorganization of some of the bonding electrons. Proton tautomers include interconversions via migration of a proton, such as keto-enol and imine-enamine isomerizations. Unless otherwise stated, all tautomers of the compounds of the disclosure are within the scope of the disclosure.

The term “isotopic substitution” as used herein refers to the substitution of an atom with its isotope. The term “isotope” as used herein refers to an atom having the same atomic number as that of atoms dominant in nature but having a mass number (neutron number) different from the mass number of the atoms dominant in nature. It is understood that a compound with an isotopic substitution refers to a compound in which at least one atom contained therein is substituted with its isotope. Atoms that can be substituted with its isotope include, but are not limited to, hydrogen, carbon, and oxygen. Examples of the isotope of a hydrogen atom include ²H (also represented as D) and ³H. Examples of the isotope of a carbon atom include ¹³C and ¹⁴C. Examples of the isotope of an oxygen atom include ¹⁸O. Unless otherwise stated, all isotopic substitution of the compounds of the disclosure are within the scope of the disclosure. Such compounds are useful, for example, as analytical tools, as probes in biological assays, or as therapeutic agents in accordance with the present disclosure.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Exemplary pharmaceutically acceptable salts are found, e.g., in Berge, et al. (J. Pharm. Sci. 1977, 66(1), 1; and Gould, P. L., Int. J. Pharmaceutics 1986, 33, 201-217; (each hereby incorporated by reference in its entirety).

Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate, and aryl sulfonate.

Pharmaceutically acceptable salts are also intended to encompass hemi-salts, wherein the ratio of compound:acid is respectively 2:1. Exemplary hemi-salts are those salts derived from acids comprising two carboxylic acid groups, such as malic acid, fumaric acid, maleic acid, succinic acid, tartaric acid, glutaric acid, oxalic acid, adipic acid and citric acid. Other exemplary hemi-salts are those salts derived from diprotic mineral acids such as sulfuric acid. Exemplary preferred hemi-salts include, but are not limited to, hemimaleate, hemifumarate, and hemisuccinate.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

An “effective amount”, “sufficient amount”, or “therapeutically effective amount” as used herein is an amount of a compound that is sufficient to effect beneficial or desired results, including clinical results. As such, the effective amount may be sufficient, e.g., to reduce or ameliorate the severity and/or duration of afflictions related to PI3Kα signaling, or one or more symptoms thereof, prevent the advancement of conditions or symptoms related to afflictions related to PI3Kα signaling, or enhance or otherwise improve the prophylactic or therapeutic effect(s) of another therapy. An effective amount also includes the amount of the compound that avoids or substantially attenuates undesirable side effects.

As used herein and as well understood in the art, “treatment” is an approach for obtaining beneficial or desired results, including clinical results. Beneficial or desired clinical results may include, but are not limited to, alleviation or amelioration of one or more symptoms or conditions, diminution of extent of disease or affliction, a stabilized (i.e., not worsening) state of disease or affliction, preventing spread of disease or affliction, delay or slowing of disease or affliction progression, amelioration or palliation of the disease or affliction state and remission (whether partial or total), whether detectable or undetectable. “Treatment” can also mean prolonging survival as compared to expected survival if not receiving treatment. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The phrase “in need thereof” refers to the need for symptomatic or asymptomatic relief from conditions related to PI3Kα signaling activity or that may otherwise be relieved by the compounds and/or compositions of the disclosure.

### 3. Description of Exemplary Embodiments

As described above, in some embodiments, the present disclosure provides a compound of formula I:

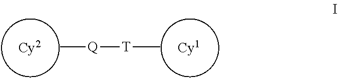

or a pharmaceutically acceptable salt thereof, wherein:
Cy¹ is phenyl; naphthyl; cubanyl; adamantyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; wherein Cy¹ is substituted with n instances of R¹;
Cy² is phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein Cy² is substituted with m instances of R²;
Q is L^{Q};
T is a bivalent C₁₋₃ aliphatic chain substituted with q instances of R^{T};
each R¹ is independently -L¹-R^{1A};
each R² is independently -L²-R^{2A};
each R^{T} is independently -L^{T}-R^{TA}; or
   two instances of R^{T} are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p¹ instances of R^{TTC};
   two instances of R¹ are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p² instances of R^{11C};
   two instances of R² are taken together with their intervening atoms to form a 3-7 membered saturated, partially unsaturated, or aromatic carbocyclic ring or a 3-7 membered saturated or partially unsaturated monocyclic heterocyclic ring having 1-3 heteroatoms independently selected from nitrogen, oxygen, and sulfur; wherein the ring is substituted with p³ instances of R^{22C};
   one instance of R^{T} and one instance of R¹ are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p⁴ instances of R^{TLC}; or
   one instance of R^{T} and one instance of R^{L} are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p⁵ instances of R^{TLC};
each of L¹, L², L^{Q}, and L^{T} is independently a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —N(R)C(NR)—, —N(R)C(NOR)—, —N(R)C(NCN)—, —C(O)N(R)—, —N(R)S(O)₂—, —S(O)₂N(R)—, —O—, —C(O)—, —OC(O)—, —C(O)O—, —S—, —S(O)—, or —S(O)₂—;
each R^{1A} is independently R^{A} or R^{B} substituted by r¹ instances of R^{1C};
each R^{2A} is independently R^{A} or R^{B} substituted by r² instances of R^{2C};
each R^{TA} is independently R^{A} or R^{B} substituted by r³ instances of R^{TC};
each R^{L} is independently R^{A} or R^{B} substituted by r⁴ instances of R^{LC};
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{1C}, R^{2C}, R^{TC}, R^{TTC}, R^{11C}, R^{22C}, R^{T1C}, R^{TLC}, and R^{LC} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and each of n, m, q, p¹, p², p³, p⁴, p⁵, r¹, r², r³, and r⁴ is independently 0, 1, 2, 3, 4, or 5.

As defined generally above, Cy¹ is phenyl; naphthyl; cubanyl; adamantyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; wherein Cy¹ is substituted with n instances of R¹.

In some embodiments, Cy¹ is phenyl, wherein Cy¹ is substituted with n instances of R¹. In some embodiments, Cy¹ is naphthyl, wherein Cy¹ is substituted with n instances of R¹. In some embodiments, Cy¹ is cubanyl, wherein Cy¹ is substituted with n instances of R¹. In some embodiments, Cy¹ is adamantyl, wherein Cy¹ is substituted with n instances of R¹. In some embodiments, Cy¹ is a 3-7 membered saturated or partially unsaturated monocyclic carbocyclic ring, wherein Cy¹ is substituted with n instances of R¹. In some embodiments, Cy¹ is a 3-7 membered saturated or partially unsaturated monocyclic heterocyclic ring having 1-2 heteroatoms independently selected from nitrogen, oxygen, and sulfur, wherein Cy¹ is substituted with n instances of R¹. In some embodiments, Cy¹ is a 5-6 membered monocyclic heteroaryl ring having 1-4 heteroatoms independently selected from nitrogen, oxygen, and sulfur, wherein Cy¹ is substituted with n instances of R¹. In some embodiments, Cy¹ is an 8-10 membered bicyclic heteroaryl ring having 1-4 heteroatoms independently selected from nitrogen, oxygen, and sulfur, wherein Cy¹ is substituted with n instances of R¹.

In some embodiments, Cy¹ is

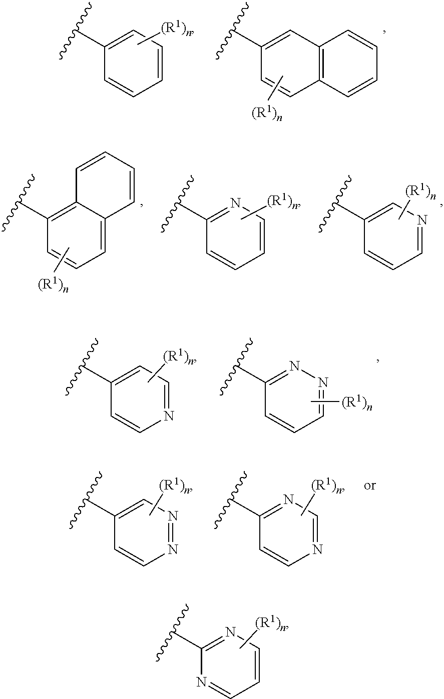

wherein R¹ and n are as defined in the embodiments and classes and subclasses herein. In some embodiments, Cy¹ is

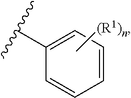

wherein R¹ and n are as defined in the embodiments and classes and subclasses herein. In some embodiments, Cy¹ is

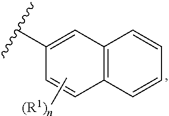

wherein R¹ and n are as defined in the embodiments and classes and subclasses herein. In some embodiments, Cy¹ is

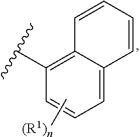

wherein R¹ and n are as defined in the embodiments and classes and subclasses herein. In some embodiments, Cy¹ is

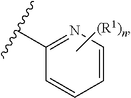

wherein R¹ and n are as defined in the embodiments and classes and subclasses herein. In some embodiments, Cy¹ is

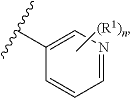

wherein R¹ and n are as defined in the embodiments and classes and subclasses herein. In some embodiments, Cy¹ is

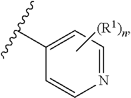

wherein R¹ and n are as defined in the embodiments and classes and subclasses herein. In some embodiments, Cy¹ is

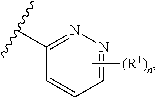

wherein R¹ and n are as defined in the embodiments and classes and subclasses herein. In some embodiments, Cy¹ is

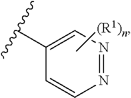

wherein R¹ and n are as defined in the embodiments and classes and subclasses herein. In some embodiments, Cy¹ is

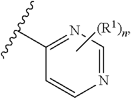

wherein R¹ and n are as defined in the embodiments and classes and subclasses herein. In some embodiments, Cy¹ is

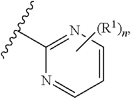

wherein R¹ and n are as defined in the embodiments and classes and subclasses herein.

In some embodiments, Cy¹ is

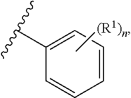

wherein R¹ is halogen and n is as defined in the embodiments and classes and subclasses herein. In some embodiments, Cy¹ is

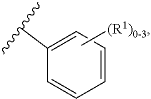

wherein R¹ is halogen. In some embodiments, Cy¹ is

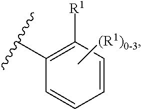

wherein R¹ is as defined in the embodiments and classes and subclasses herein. In some embodiments, Cy¹ is

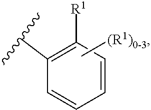

wherein R¹ is halogen. In some embodiments, Cy¹ is

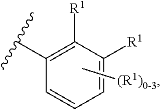

wherein R¹ is as defined in the embodiments and classes and subclasses herein. In some embodiments, Cy¹ is

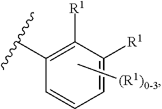

wherein R¹ is halogen. In some embodiments, Cy¹ is

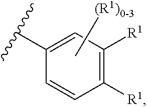

wherein R¹ is as defined in the embodiments and classes and subclasses herein. In some embodiments, Cy¹ is

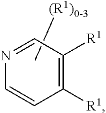

wherein R¹ is halogen. In some embodiments, Cy¹ is

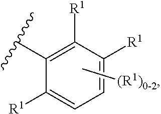

wherein R¹ is as defined in the embodiments and classes and subclasses herein. In some embodiments, Cy¹ is

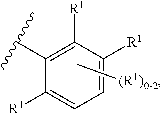

wherein R¹ is halogen.

In some embodiments, Cy¹ is

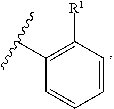

wherein R¹ is as defined in the embodiments and classes and subclasses herein. In some embodiments, Cy¹ is

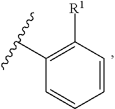

wherein R¹ is halogen. In some embodiments, Cy¹ is

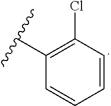 [SMILES: *c1ccccc1Cl]

In some embodiments, Cy¹ is

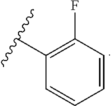 [SMILES: *c1ccccc1F]

In some embodiments, Cy¹ is

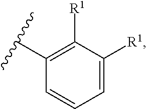

wherein R¹ is as defined in the embodiments and classes and subclasses herein. In some embodiments, Cy¹ is

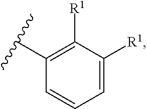

wherein R¹ is halogen. In some embodiments, Cy¹ is

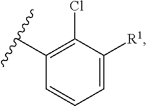

wherein R¹ is halogen. In some embodiments, Cy¹ is

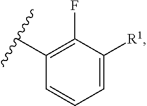

wherein R¹ is halogen. In some embodiments, Cy¹ is

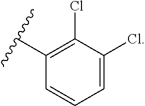 [SMILES: *c1cccc(Cl)c1Cl]

In some embodiments, Cy¹ is

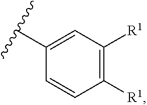

wherein R¹ is as defined in the embodiments and classes and subclasses herein. In some embodiments, Cy¹ is

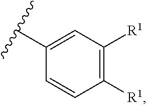

wherein R¹ is halogen. In some embodiments, Cy¹ is

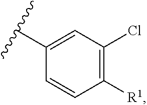

wherein R¹ is halogen. In some embodiments, Cy¹ is

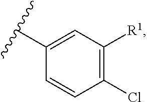

wherein R¹ is halogen. In some embodiments, Cy¹ is

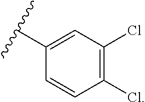 [SMILES: *c1ccc(Cl)c(Cl)c1]

In some embodiments, Cy¹ is

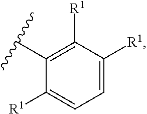

wherein R¹ is as defined in the embodiments and classes and subclasses herein. In some embodiments, Cy¹ is

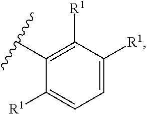

wherein R¹ is halogen. In some embodiments, Cy¹ is

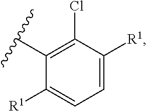

wherein R¹ is halogen. In some embodiments, Cy¹ is

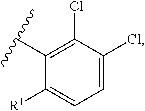

wherein R¹ is halogen. In some embodiments, Cy¹ is

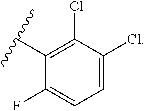 [SMILES: *c1c(F)ccc(Cl)c1Cl]

In some embodiments, Cy¹ is

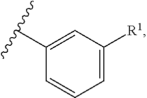

wherein R¹ is as defined in the embodiments and classes and subclasses herein. In some embodiments, Cy¹ is

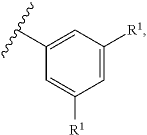

wherein R¹ is as defined in the embodiments and classes and subclasses herein. In some embodiments, Cy, is

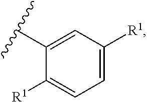

wherein R¹ is as defined in the embodiments and classes and subclasses herein. In some embodiments, Cy¹ is

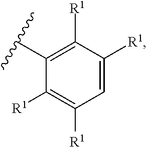

wherein R¹ is as defined in the embodiments and classes and subclasses herein. In some embodiments, Cy¹ is

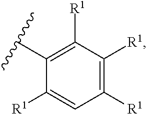

wherein R¹ is as defined in the embodiments and classes and subclasses herein. In some embodiments, Cy¹ is

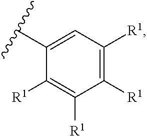

wherein R¹ is as defined in the embodiments and classes and subclasses herein. In some embodiments, Cy¹ is

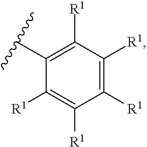

wherein R¹ is as defined in the embodiments and classes and subclasses herein.

In some embodiments, Cy¹ is an 8-10 membered bicyclic heteroaryl ring having 1-4 heteroatoms independently selected from nitrogen, oxygen, and sulfur; wherein Cy¹ is substituted with n instances of R¹ wherein R¹ is as defined in the embodiments and classes and subclasses herein. In some embodiments, Cy¹ is an 8-10 membered bicyclic heteroaryl ring having 1-2 heteroatoms independently selected from nitrogen, oxygen, and sulfur; wherein Cy¹ is substituted with n instances of R¹ wherein R¹ is as defined in the embodiments and classes and subclasses herein. In some embodiments, Cy¹ is

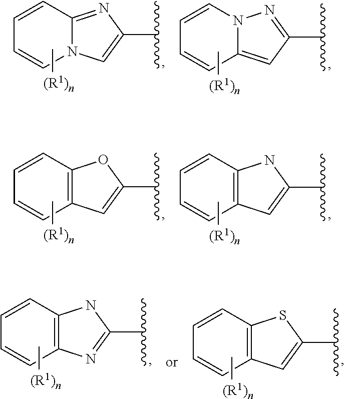

wherein n and R¹ are as defined in the embodiments and classes and subclasses herein. In some embodiments, Cy¹ is

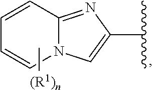

wherein n and R¹ are as defined in the embodiments and classes and subclasses herein. In some embodiments, Cy¹ is

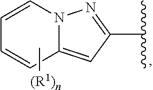

wherein n and R¹ are as defined in the embodiments and classes and subclasses herein. In some embodiments, Cy¹ is

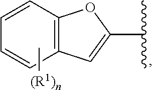

wherein n and R¹ are as defined in the embodiments and classes and subclasses herein. In some embodiments, Cy¹ is

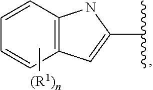

wherein n and R¹ are as defined in the embodiments and classes and subclasses herein. In some embodiments, Cy¹ is

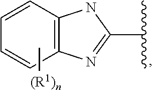

wherein n and R¹ are as defined in the embodiments and classes and subclasses herein. In some embodiments, Cy¹ is

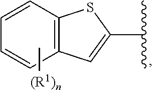

wherein n and R¹ are as defined in the embodiments and classes and subclasses herein. In some embodiments, Cy¹ is

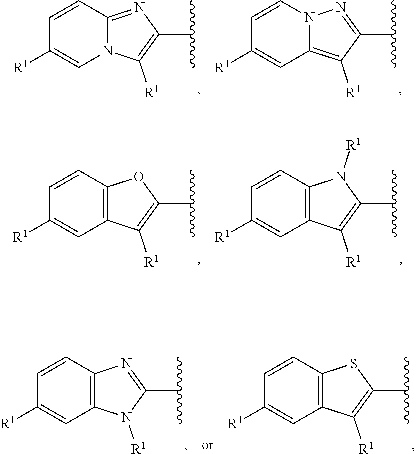

wherein n and R¹ are as defined in the embodiments and classes and subclasses herein. In some embodiments, Cy¹ is

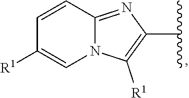

wherein n and R¹ are as defined in the embodiments and classes and subclasses herein. In some embodiments, Cy, is,

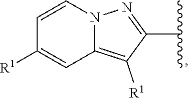

wherein n and R¹ are as defined in the embodiments and classes and subclasses herein. In some embodiments, Cy¹ is

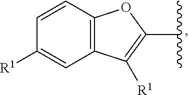

wherein n and R¹ are as defined in the embodiments and classes and subclasses herein. In some embodiments, Cy¹ is

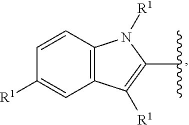

wherein n and R¹ are as defined in the embodiments and classes and subclasses herein. In some embodiments, Cy¹ is

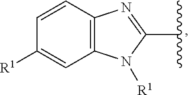

wherein n and R¹ are as defined in the embodiments and classes and subclasses herein. In some embodiments, Cy¹ is,

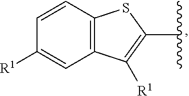

wherein n and R¹ are as defined in the embodiments and classes and subclasses herein. In some embodiments, Cy¹ is

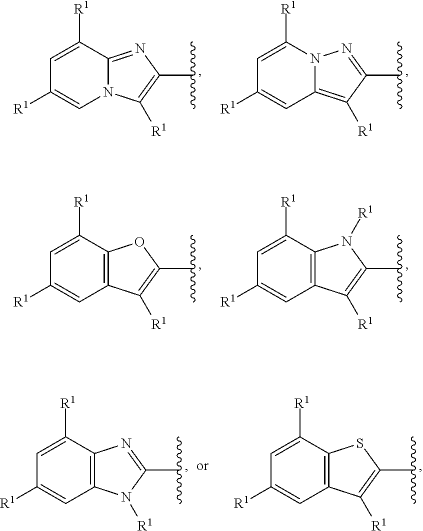

wherein n and R¹ are as defined in the embodiments and classes and subclasses herein. In some embodiments, Cy¹ is

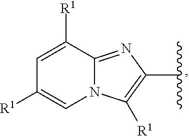

wherein n and R¹ are as defined in the embodiments and classes and subclasses herein. In some embodiments, Cy¹ is

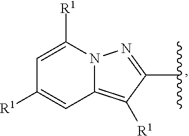

wherein n and R¹ are as defined in the embodiments and classes and subclasses herein. In some embodiments, Cy¹ is

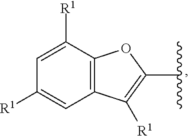

wherein n and R¹ are as defined in the embodiments and classes and subclasses herein. In some embodiments, Cy¹ is

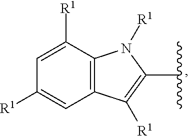

wherein n and R¹ are as defined in the embodiments and classes and subclasses herein. In some embodiments, Cy¹ is

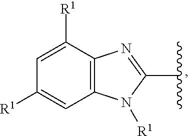

wherein n and R¹ are as defined in the embodiments and classes and subclasses herein. In some embodiments, Cy¹ is

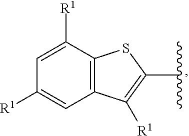

wherein n and R¹ are as defined in the embodiments and classes and subclasses herein.

In some embodiments, Cy¹ is selected from the groups depicted in the compounds in Table 1. In some embodiments, Cy¹ is not

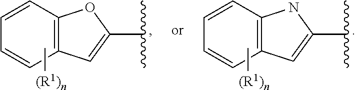

As defined generally above, Cy² is phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein Cy² is substituted with m instances of R².

In some embodiments, Cy² is phenyl, wherein Cy² is substituted with m instances of R². In some embodiments, Cy² is naphthyl, wherein Cy² is substituted with m instances of R². In some embodiments, Cy² is cubanyl, wherein Cy² is substituted with m instances of R². In some embodiments, Cy² is adamantyl, wherein Cy² is substituted with m instances of R². In some embodiments, Cy² is a 5-6 membered monocyclic heteroaryl ring having 1-4 heteroatoms independently selected from nitrogen, oxygen, and sulfur, wherein Cy² is substituted with m instances of R². In some embodiments, Cy² is an 8-10 membered bicyclic heteroaryl ring having 1-4 heteroatoms independently selected from nitrogen, oxygen, and sulfur, wherein Cy² is substituted with m instances of R². In some embodiments, Cy² is a 3-7 membered saturated or partially unsaturated monocyclic carbocyclic ring, wherein Cy² is substituted with m instances of R². In some embodiments, Cy² is a 5-12 membered saturated or partially unsaturated bicyclic carbocyclic ring, wherein Cy² is substituted with m instances of R². In some embodiments, Cy² is a 3-7 membered saturated or partially unsaturated monocyclic heterocyclic ring having 1-2 heteroatoms independently selected from nitrogen, oxygen, and sulfur, wherein Cy² is substituted with m instances of R². In some embodiments, Cy² is a 7-12 membered saturated or partially unsaturated bicyclic heterocyclic ring having 1-4 heteroatoms independently selected from nitrogen, oxygen, and sulfur, wherein Cy² is substituted with m instances of R².

In some embodiments, Cy² is an 8-10 membered bicyclic heteroaryl ring having 1-4 heteroatoms independently selected from nitrogen, oxygen, and sulfur, wherein Cy² is substituted with m instances of R². In some embodiments, Cy² is a 9-10 membered bicyclic heteroaryl ring having 1-4 heteroatoms independently selected from nitrogen, oxygen, and sulfur, wherein Cy² is substituted with m instances of R². In some embodiments, Cy² is an 8-9 membered bicyclic heteroaryl ring having 1-4 heteroatoms independently selected from nitrogen, oxygen, and sulfur, wherein Cy² is substituted with m instances of R². In some embodiments, Cy² is an 8-membered bicyclic heteroaryl ring having 1-4 heteroatoms independently selected from nitrogen, oxygen, and sulfur, wherein Cy² is substituted with m instances of R². In some embodiments, Cy² is a 9-membered bicyclic heteroaryl ring having 1-4 heteroatoms independently selected from nitrogen, oxygen, and sulfur, wherein Cy² is substituted with m instances of R². In some embodiments, Cy² is a 10-membered bicyclic heteroaryl ring having 1-4 heteroatoms independently selected from nitrogen, oxygen, and sulfur, wherein Cy² is substituted with m instances of R².

In some embodiments, Cy² is a 3-7 membered saturated or partially unsaturated monocyclic carbocyclic ring, wherein Cy² is substituted with m instances of R². In some embodiments, Cy² is a 4-6 membered saturated or partially unsaturated monocyclic carbocyclic ring, wherein Cy² is substituted with m instances of R². In some embodiments, Cy² is a 4-5 membered saturated or partially unsaturated monocyclic carbocyclic ring, wherein Cy² is substituted with m instances of R². In some embodiments, Cy² is a 5-6 membered saturated or partially unsaturated monocyclic carbocyclic ring, wherein Cy² is substituted with m instances of R². In some embodiments, Cy² is a 4-membered saturated or partially unsaturated monocyclic carbocyclic ring, wherein Cy² is substituted with m instances of R². In some embodiments, Cy² is a 5-membered saturated or partially unsaturated monocyclic carbocyclic ring, wherein Cy² is substituted with m instances of R². In some embodiments, Cy² is a 6-membered saturated or partially unsaturated monocyclic carbocyclic ring, wherein Cy² is substituted with m instances of R.

In some embodiments, Cy² is

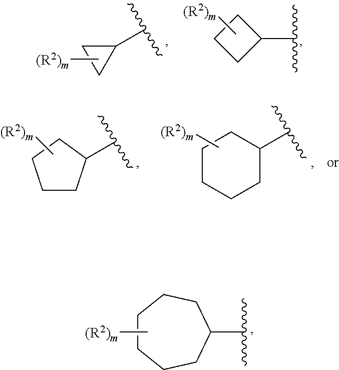

wherein R² and m are as defined in embodiments and classes and subclasses herein. In some embodiments, Cy² is

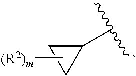

wherein R² and m are as defined in embodiments and classes and subclasses herein. In some embodiments, Cy² is

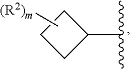

wherein R² and m are as defined in embodiments and classes and subclasses herein. In some embodiments, Cy² is

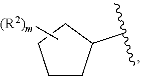

wherein R² and m are as defined in embodiments and classes and subclasses herein. In some embodiments, Cy² is

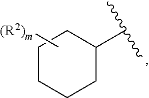

wherein R² and m are as defined in embodiments and classes and subclasses herein. In some embodiments, Cy² is

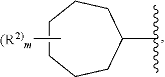

wherein R² and m are as defined in embodiments and classes and subclasses herein.

In some embodiments, Cy² is

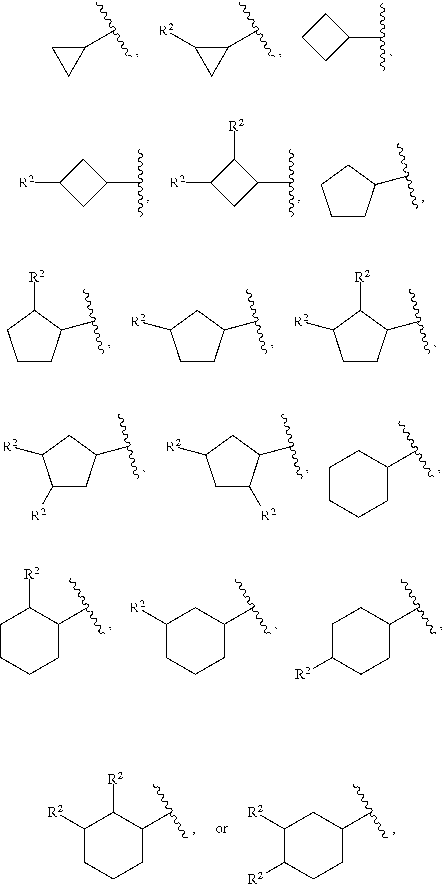

wherein R² is as defined in embodiments and classes and subclasses herein. In some embodiments, Cy² is

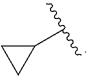 [SMILES: *C1CC1]

In some embodiments, Cy² is

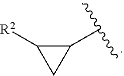

wherein R² is as defined in embodiments and classes and subclasses herein. In some embodiments, Cy² is

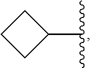

wherein R² is as defined in embodiments and classes and subclasses herein. In some embodiments, Cy² is

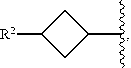

wherein R² is as defined in embodiments and classes and subclasses herein. In some embodiments, Cy² is

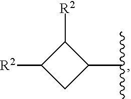

wherein R² is as defined in embodiments and classes and subclasses herein. In some embodiments, Cy² is

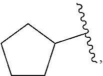

wherein R² is as defined in embodiments and classes and subclasses herein. In some embodiments, Cy² is

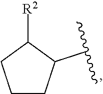

wherein R² is as defined in embodiments and classes and subclasses herein. In some embodiments, Cy² is

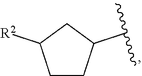

wherein R² is as defined in embodiments and classes and subclasses herein. In some embodiments, Cy² is

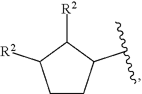

wherein R² is as defined in embodiments and classes and subclasses herein. In some embodiments, Cy² is

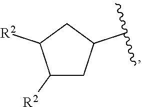

wherein R² is as defined in embodiments and classes and subclasses herein. In some embodiments, Cy² is

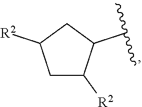

wherein R² is as defined in embodiments and classes and subclasses herein. In some embodiments, Cy² is

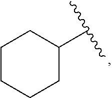

wherein R² is as defined in embodiments and classes and subclasses herein. In some embodiments, Cy² is

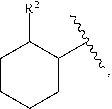

wherein R² is as defined in embodiments and classes and subclasses herein. In some embodiments, Cy² is

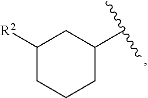

wherein R² is as defined in embodiments and classes and subclasses herein. In some embodiments, Cy² is

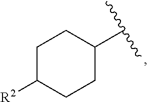

wherein R² is as defined in embodiments and classes and subclasses herein. In some embodiments, Cy² is

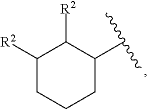

wherein R² is as defined in embodiments and classes and subclasses herein. In some embodiments, Cy² is

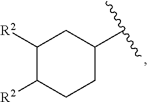

wherein R² is as defined in embodiments and classes and subclasses herein.

In some embodiments, Cy² is a 7-12 membered saturated or partially unsaturated bicyclic heterocyclic ring having 1-4 heteroatoms independently selected from nitrogen, oxygen, and sulfur, wherein Cy² is substituted with m instances of R². In some embodiments, Cy² is an 8-10 membered saturated or partially unsaturated bicyclic heterocyclic ring having 1-4 heteroatoms independently selected from nitrogen, oxygen, and sulfur, wherein Cy² is substituted with m instances of R². In some embodiments, Cy² is an 8-9 membered saturated or partially unsaturated bicyclic heterocyclic ring having 1-4 heteroatoms independently selected from nitrogen, oxygen, and sulfur, wherein Cy² is substituted with m instances of R². In some embodiments, Cy² is an 8-membered saturated or partially unsaturated bicyclic heterocyclic ring having 1-4 heteroatoms independently selected from nitrogen, oxygen, and sulfur, wherein Cy² is substituted with m instances of R². In some embodiments, Cy² is a 9-membered saturated or partially unsaturated bicyclic heterocyclic ring having 1-4 heteroatoms independently selected from nitrogen, oxygen, and sulfur, wherein Cy² is substituted with m instances of R².

In some embodiments, Cy² is

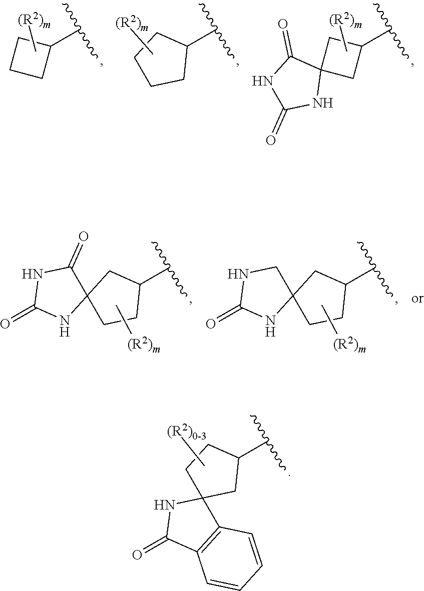

In some embodiments, Cy² is

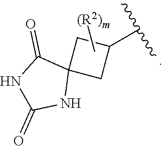

In some embodiments, Cy² is

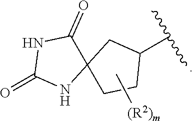

In some embodiments, Cy² is

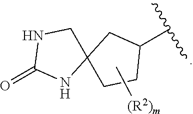

In some embodiments, Cy² is

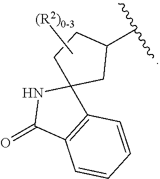

In some embodiments, Cy² is

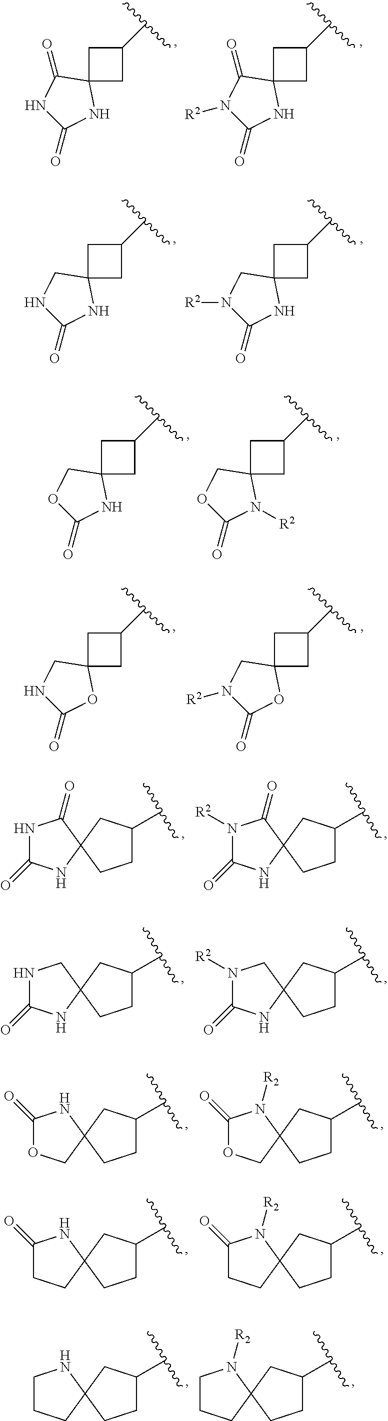

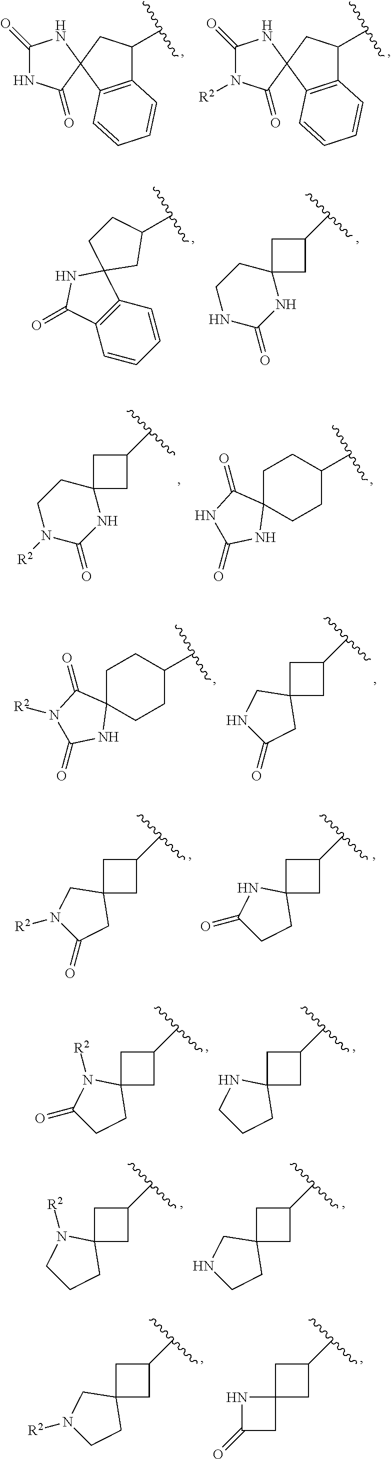

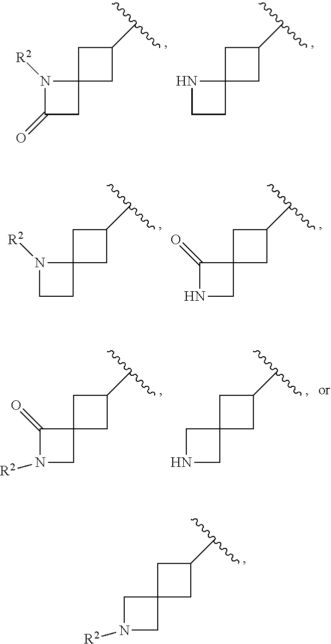

wherein R² is as defined in embodiments and classes and subclasses herein.

In some embodiments, Cy² is

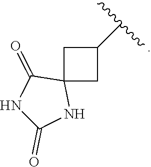 [SMILES: *C1CC2(C1)NC(=O)NC2=O]

In some embodiments, Cy² is

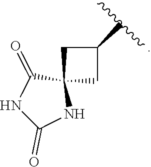 [SMILES: *[C@H]1C[C@]2(C1)NC(=O)NC2=O]

In some embodiments, Cy² is

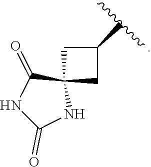 [SMILES: *[C@H]1C[C@@]2(C1)NC(=O)NC2=O]

In some embodiments, Cy² is

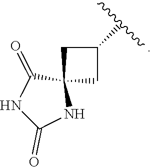 [SMILES: *[C@H]1C[C@@]2(C1)NC(=O)NC2=O]

In some embodiments, Cy² is

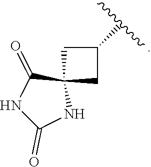 [SMILES: *[C@H]1C[C@]2(C1)NC(=O)NC2=O]

In some embodiments, Cy² is

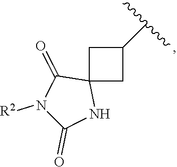

wherein R² is as defined in embodiments and classes and subclasses herein. In some embodiments, Cy² is

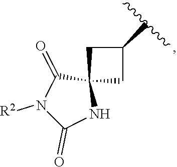

wherein R² is as defined in embodiments and classes and subclasses herein. In some embodiments, Cy² is

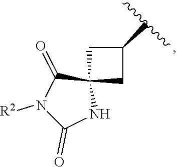

wherein R² is as defined in embodiments and classes and subclasses herein. In some embodiments, Cy² is

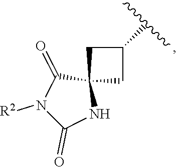

wherein R² is as defined in embodiments and classes and subclasses herein. In some embodiments, Cy² is

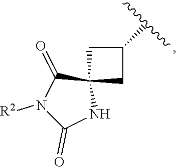

wherein R² is as defined in embodiments and classes and subclasses herein.

In some embodiments, Cy² is

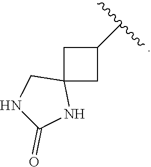 [SMILES: *C1CC2(CNC(=O)N2)C1]

In some embodiments, Cy² is

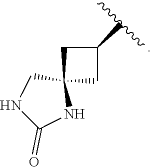 [SMILES: *[C@H]1C[C@]2(CNC(=O)N2)C1]

In some embodiments, Cy² is

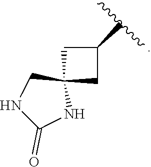 [SMILES: *[C@H]1C[C@@]2(CNC(=O)N2)C1]

In some embodiments, Cy² is

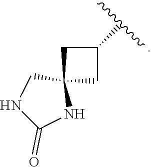 [SMILES: *[C@H]1C[C@@]2(CNC(=O)N2)C1]

In some embodiments, Cy² is

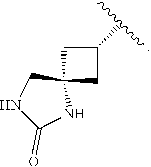 [SMILES: *[C@H]1C[C@]2(CNC(=O)N2)C1]

In some embodiments, Cy² is

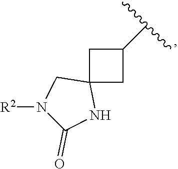

wherein R² is as defined in embodiments and classes and subclasses herein. In some embodiments, Cy² is

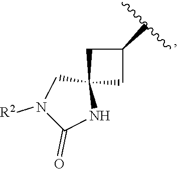

wherein R² is as defined in embodiments and classes and subclasses herein. In some embodiments, Cy² is

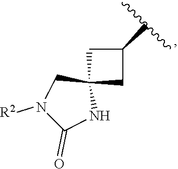

wherein R² is as defined in embodiments and classes and subclasses herein. In some embodiments, Cy² is

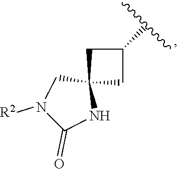

wherein R² is as defined in embodiments and classes and subclasses herein. In some embodiments, Cy² is

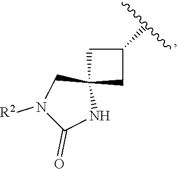

wherein R² is as defined in embodiments and classes and subclasses herein.

In some embodiments, Cy¹ is

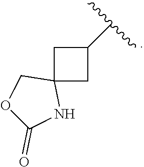 [SMILES: *C1CC2(COC(=O)N2)C1]

In some embodiments, Cy² is

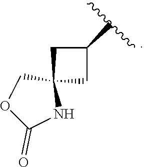 [SMILES: *[C@H]1C[C@]2(COC(=O)N2)C1]

In some embodiments, Cy² is

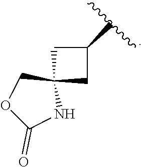 [SMILES: *[C@H]1C[C@@]2(COC(=O)N2)C1]

In some embodiments, Cy² is

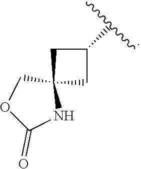 [SMILES: *[C@H]1C[C@@]2(COC(=O)N2)C1]

In some embodiments, Cy² is

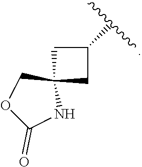 [SMILES: *[C@H]1C[C@]2(COC(=O)N2)C1]

In some embodiments, Cy² is

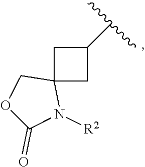

wherein R² is as defined in embodiments and classes and subclasses herein. In some embodiments, Cy² is

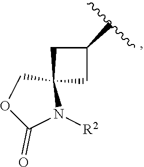

herein R² is as defined in embodiments and classes and subclasses herein. In some embodiments, Cy² is

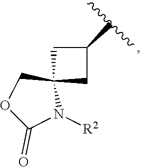

wherein R² is as defined in embodiments and classes and subclasses herein. In some embodiments, Cy² is

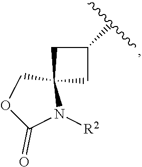

wherein R² is as defined in embodiments and classes and subclasses herein. In some embodiments, Cy² is

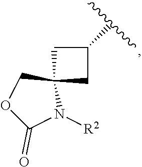

wherein R² is as defined in embodiments and classes and subclasses herein.

In some embodiments, Cy² is

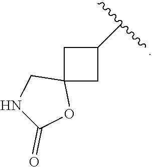 [SMILES: *C1CC2(CNC(=O)O2)C1]

In some embodiments, Cy² is

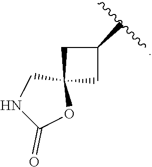 [SMILES: *[C@H]1C[C@]2(CNC(=O)O2)C1]

In some embodiments, Cy² is

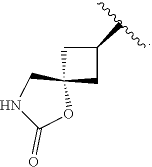 [SMILES: *[C@H]1C[C@@]2(CNC(=O)O2)C1]

In some embodiments, Cy² is

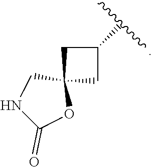 [SMILES: *[C@H]1C[C@@]2(CNC(=O)O2)C1]

In some embodiments, Cy² is

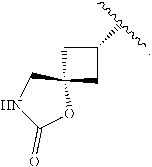 [SMILES: *[C@H]1C[C@]2(CNC(=O)O2)C1]

In some embodiments, Cy² is

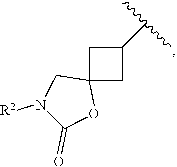

wherein R² is as defined in embodiments and classes and subclasses herein. In some embodiments, Cy² is

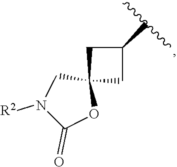

wherein R² is as defined in embodiments and classes and subclasses herein.

In some embodiments, Cy² is

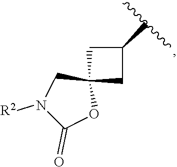

wherein R² is as defined in embodiments and classes and subclasses herein. In some embodiments, Cy² is

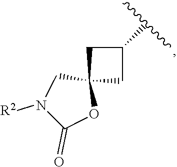

wherein R² is as defined in embodiments and classes and subclasses herein. In some embodiments, Cy² is

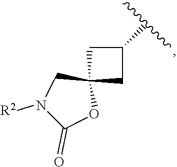

wherein R² is as defined in embodiments and classes and subclasses herein.

In some embodiments, Cy² is

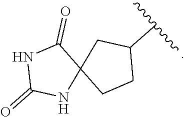 [SMILES: *C1CCC2(C1)NC(=O)NC2=O]

In some embodiments, Cy² is

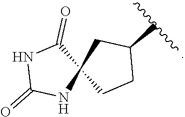 [SMILES: *[C@H]1CC[C@]2(C1)NC(=O)NC2=O]

In some embodiments, Cy² is

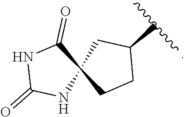 [SMILES: *[C@H]1CC[C@@]2(C1)NC(=O)NC2=O]

In some embodiments, Cy² is

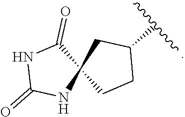 [SMILES: *[C@@H]1CC[C@]2(C1)NC(=O)NC2=O]

In some embodiments, Cy² is

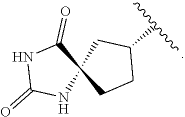 [SMILES: *[C@@H]1CC[C@@]2(C1)NC(=O)NC2=O]

In some embodiments, Cy² is

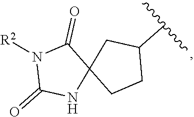

wherein R² is as defined in embodiments and classes and subclasses herein. In some embodiments, Cy² is

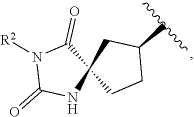

wherein R² is as defined in embodiments and classes and subclasses herein. In some embodiments, Cy² is

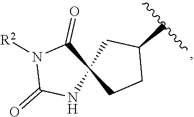

wherein R² is as defined in embodiments and classes and subclasses herein. In some embodiments, Cy² is

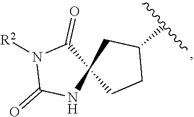

wherein R² is as defined in embodiments and classes and subclasses herein. In some embodiments, Cy² is

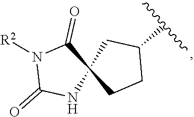

wherein R² is as defined in embodiments and classes and subclasses herein.

In some embodiments, Cy² is

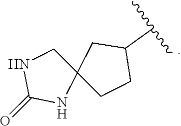 [SMILES: *C1CCC2(CNC(=O)N2)C1]

In some embodiments, Cy² is

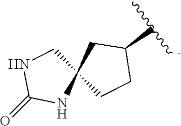 [SMILES: *[C@H]1CC[C@]2(CNC(=O)N2)C1]

In some embodiments, Cy² is

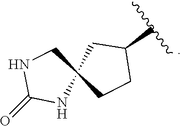 [SMILES: *[C@H]1CC[C@@]2(CNC(=O)N2)C1]

In some embodiments, Cy² is

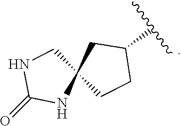 [SMILES: *[C@@H]1CC[C@]2(CNC(=O)N2)C1]

In some embodiments, Cy² is

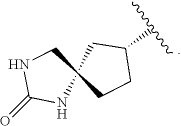 [SMILES: *[C@@H]1CC[C@@]2(CNC(=O)N2)C1]

In some embodiments, Cy² is

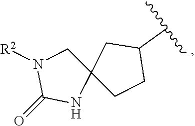

wherein R² is as defined in embodiments and classes and subclasses herein. In some embodiments, Cy² is

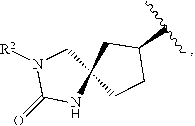

wherein R² is as defined in embodiments and classes and subclasses herein. In some embodiments, Cy² is

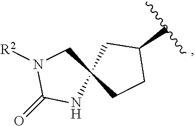

wherein R² is as defined in embodiments and classes and subclasses herein. In some embodiments, Cy² is

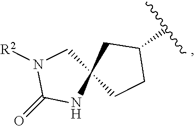

wherein R² is as defined in embodiments and classes and subclasses herein. In some embodiments, Cy² is

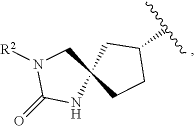

wherein R² is as defined in embodiments and classes and subclasses herein.

In some embodiments, Cy² is

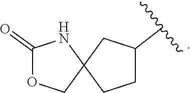 [SMILES: *C1CCC2(COC(=O)N2)C1]

In some embodiments, Cy² is

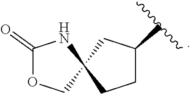 [SMILES: *[C@H]1CC[C@]2(COC(=O)N2)C1]

In some embodiments, Cy² is

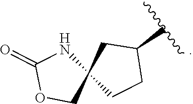 [SMILES: *[C@H]1CC[C@@]2(COC(=O)N2)C1]

In some embodiments, Cy² is

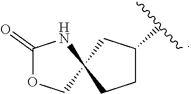 [SMILES: *[C@@H]1CC[C@]2(COC(=O)N2)C1]

In some embodiments, Cy² is

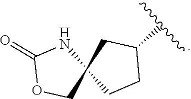 [SMILES: *[C@@H]1CC[C@@]2(COC(=O)N2)C1]

In some embodiments, Cy² is

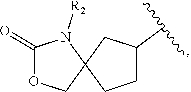

wherein R² is as defined in embodiments and classes and subclasses herein. In some embodiments, Cy² is

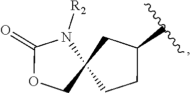

wherein R² is as defined in embodiments and classes and subclasses herein. In some embodiments, Cy² is

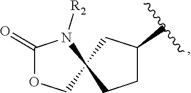

wherein R² is as defined in embodiments and classes and subclasses herein. In some embodiments, Cy² is

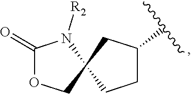

wherein R² is as defined in embodiments and classes and subclasses herein. In some embodiments, Cy² is

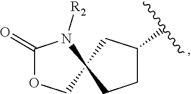

wherein R² is as defined in embodiments and classes and subclasses herein. In some embodiments, Cy² is

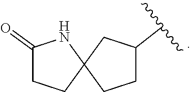 [SMILES: *C1CCC2(CCC(=O)N2)C1]

In some embodiments, Cy² is

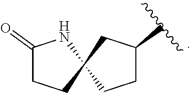 [SMILES: *[C@H]1CC[C@@]2(CCC(=O)N2)C1]

In some embodiments, Cy² is

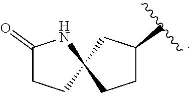 [SMILES: *[C@H]1CC[C@]2(CCC(=O)N2)C1]

In some embodiments, Cy² is

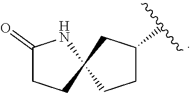 [SMILES: *[C@@H]1CC[C@@]2(CCC(=O)N2)C1]

In some embodiments, Cy² is

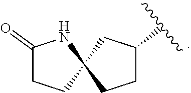 [SMILES: *[C@@H]1CC[C@]2(CCC(=O)N2)C1]

In some embodiments, Cy² is

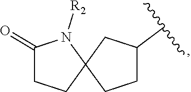

wherein R² is as defined in embodiments and classes and subclasses herein. In some embodiments, Cy² is

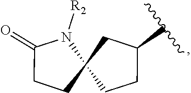

wherein R² is as defined in embodiments and classes and subclasses herein. In some embodiments, Cy² is

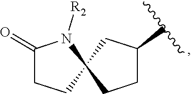

wherein R² is as defined in embodiments and classes and subclasses herein. In some embodiments, Cy² is

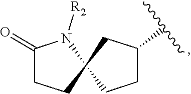

wherein R² is as defined in embodiments and classes and subclasses herein. In some embodiments, Cy² is

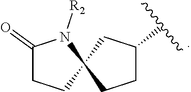

wherein R² is as defined in embodiments and classes and subclasses herein.

In some embodiments, Cy² is

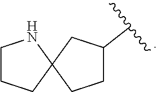 [SMILES: *C1CCC2(CCCN2)C1]

In some embodiments, Cy² is

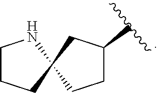 [SMILES: *[C@H]1CC[C@@]2(CCCN2)C1]

In some embodiments, Cy² is

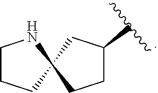 [SMILES: *[C@H]1CC[C@]2(CCCN2)C1]

In some embodiments, Cy² is

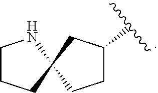 [SMILES: *[C@@H]1CC[C@@]2(CCCN2)C1]

In some embodiments, Cy² is

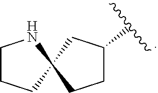 [SMILES: *[C@@H]1CC[C@]2(CCCN2)C1]

In some embodiments, Cy² is

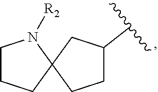

wherein R² is as defined in embodiments and classes and subclasses herein. In some embodiments, Cy² is

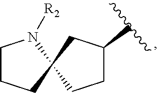

wherein R² is as defined in embodiments and classes and subclasses herein. In some embodiments, Cy² is

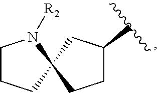

wherein R² is as defined in embodiments and classes and subclasses herein. In some embodiments, Cy² is

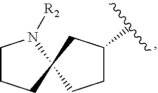

wherein R² is as defined in embodiments and classes and subclasses herein. In some embodiments, Cy² is

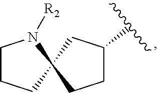

wherein R² is as defined in embodiments and classes and subclasses herein.

In some embodiments, Cy² is

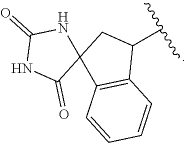 [SMILES: *C1CC2(NC(=O)NC2=O)c2ccccc21]

In some embodiments, Cy² is

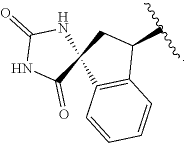 [SMILES: *[C@@H]1C[C@@]2(NC(=O)NC2=O)c2ccccc21]

In some embodiments, Cy² is

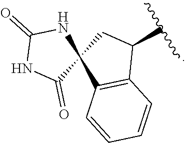 [SMILES: *[C@@H]1C[C@]2(NC(=O)NC2=O)c2ccccc21]

In some embodiments, Cy² is

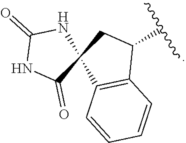 [SMILES: *[C@H]1C[C@@]2(NC(=O)NC2=O)c2ccccc21]

In some embodiments, Cy² is

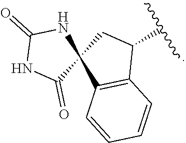 [SMILES: *[C@H]1C[C@]2(NC(=O)NC2=O)c2ccccc21]

In some embodiments, Cy² is

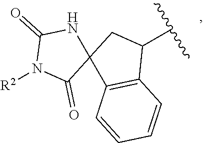

wherein R² is as defined in embodiments and classes and subclasses herein. In some embodiments, Cy² is

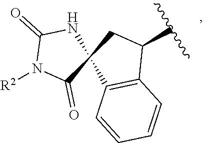

herein R² is as defined in embodiments and classes and subclasses herein. In some embodiments, Cy² is

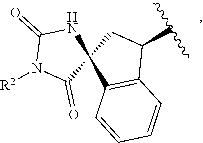

wherein R² is as defined in embodiments and classes and subclasses herein. In some embodiments, Cy² is

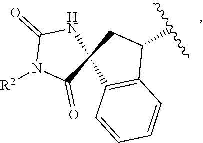

wherein R² is as defined in embodiments and classes and subclasses herein. In some embodiments, Cy² is

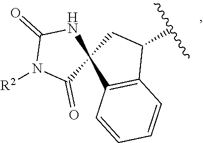

wherein R² is a defined in embodiments and classes and subclasses herein.

In some embodiments, Cy² is

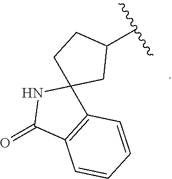 [SMILES: *C1CCC2(C1)NC(=O)c1ccccc12]

In some embodiments, Cy² is

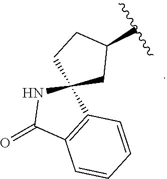 [SMILES: *[C@@H]1CC[C@@]2(C1)NC(=O)c1ccccc12]

In some embodiments, Cy² is

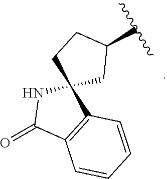 [SMILES: *[C@@H]1CC[C@]2(C1)NC(=O)c1ccccc12]

In some embodiments, Cy² is

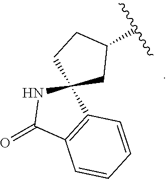 [SMILES: *[C@H]1CC[C@@]2(C1)NC(=O)c1ccccc12]

In some embodiments, Cy² is

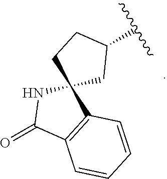 [SMILES: *[C@H]1CC[C@]2(C1)NC(=O)c1ccccc12]

In some embodiments, Cy² is

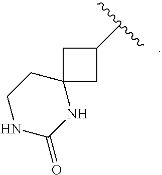 [SMILES: *C1CC2(CCNC(=O)N2)C1]

In some embodiments, Cy² is

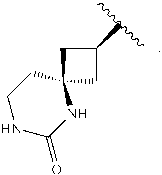 [SMILES: *[C@H]1C[C@]2(CCNC(=O)N2)C1]

In some embodiments, Cy² is

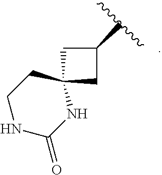 [SMILES: *[C@H]1C[C@@]2(CCNC(=O)N2)C1]

In some embodiments, Cy² is

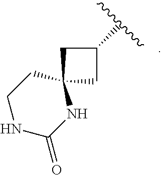 [SMILES: *[C@H]1C[C@@]2(CCNC(=O)N2)C1]

In some embodiments, Cy² is

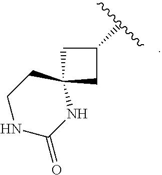 [SMILES: *[C@H]1C[C@]2(CCNC(=O)N2)C1]

In some embodiments, Cy² is

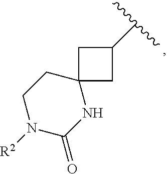

wherein R² is as defined in embodiments and classes and subclasses herein. In some embodiments, Cy² is

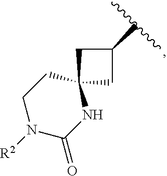

wherein R² is as defined in embodiments and classes and subclasses herein. In some embodiments, Cy² is

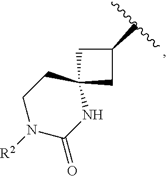

wherein R² is as defined in embodiments and classes and subclasses herein. In some embodiments, Cy² is

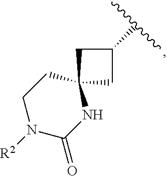

wherein R² is as defined in embodiments and classes and subclasses herein. In some embodiments, Cy² is

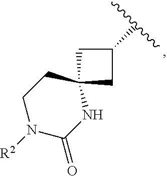

wherein R² is as defined in embodiments and classes and subclasses herein.

In some embodiments, Cy² is

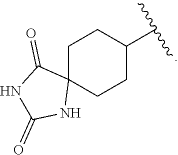 [SMILES: *C1CCC2(CC1)NC(=O)NC2=O]

In some embodiments, Cy² is

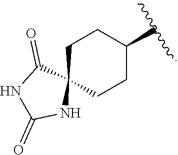 [SMILES: *[C@H]1CC[C@]2(CC1)NC(=O)NC2=O]

In some embodiments, Cy² is

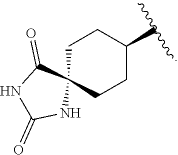 [SMILES: *[C@H]1CC[C@@]2(CC1)NC(=O)NC2=O]

In some embodiments, Cy² is

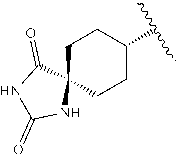 [SMILES: *[C@H]1CC[C@@]2(CC1)NC(=O)NC2=O]

In some embodiments, Cy² is

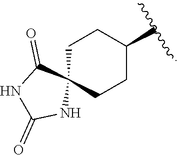 [SMILES: *[C@H]1CC[C@@]2(CC1)NC(=O)NC2=O]

In some embodiments, Cy² is

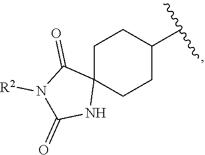

wherein R² is as defined in embodiments and classes and subclasses herein. In some embodiments, Cy² is

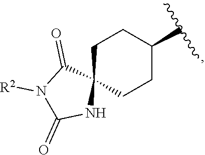

wherein R² is as defined in embodiments and classes and subclasses herein. In some embodiments, Cy² is

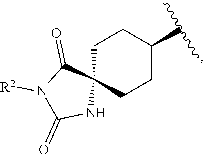

wherein R² is as defined in embodiments and classes and subclasses herein. In some embodiments, Cy² is

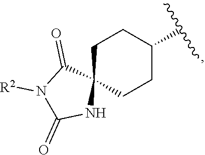

wherein R² is as defined in embodiments and classes and subclasses herein. In some embodiments, Cy² is

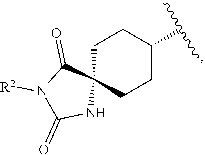

wherein R² is as defined in embodiments and classes and subclasses herein.

In some embodiments, Cy² is

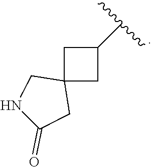 [SMILES: *C1CC2(CNC(=O)C2)C1]

In some embodiments, Cy² is

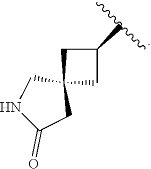 [SMILES: *[C@H]1C[C@]2(CNC(=O)C2)C1]

In some embodiments, Cy² is

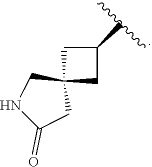 [SMILES: *[C@H]1C[C@@]2(CNC(=O)C2)C1]

In some embodiments, Cy² is

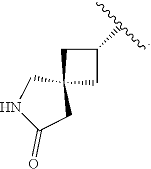 [SMILES: *[C@H]1C[C@@]2(CNC(=O)C2)C1]

In some embodiments, Cy² is

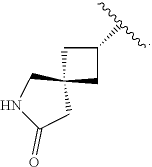 [SMILES: *[C@H]1C[C@]2(CNC(=O)C2)C1]

In some embodiments, Cy² is

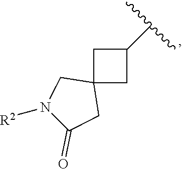

wherein R² is as defined in embodiments and classes and subclasses herein. In some embodiments, Cy² is

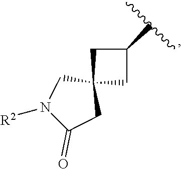

wherein R² is as defined in embodiments and classes and subclasses herein. In some embodiments, Cy² is

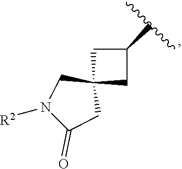

wherein R² is as defined in embodiments and classes and subclasses herein. In some embodiments, Cy² is

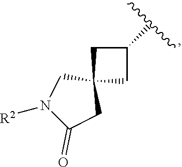

wherein R² is as defined in embodiments and classes and subclasses herein. In some embodiments, Cy² is

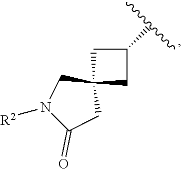

wherein R² is as defined in classes and subclasses herein.

In some embodiments, Cy² is

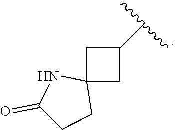 [SMILES: *C1CC2(CCC(=O)N2)C1]

In some embodiments, Cy² is

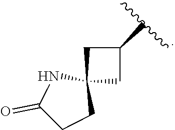 [SMILES: *[C@H]1C[C@@]2(CCC(=O)N2)C1]

In some embodiments, Cy² is

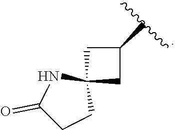 [SMILES: *[C@H]1C[C@]2(CCC(=O)N2)C1]

In some embodiments, Cy² is

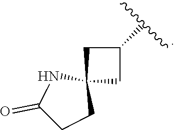 [SMILES: *[C@H]1C[C@]2(CCC(=O)N2)C1]

In some embodiments, Cy² is

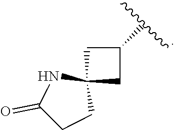 [SMILES: *[C@H]1C[C@@]2(CCC(=O)N2)C1]

In some embodiments, Cy² is

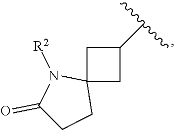

wherein R² is as defined in embodiments and classes and subclasses herein. In some embodiments, Cy² is

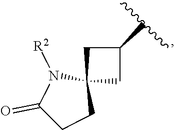

wherein R² is as defined in embodiments and classes and subclasses herein. In some embodiments, Cy² is

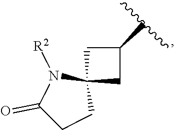

wherein R² is as defined in embodiments and classes and subclasses herein. In some embodiments, Cy² is

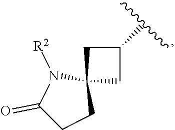

wherein R² is as defined in embodiments and classes and subclasses herein. In some embodiments, Cy² is

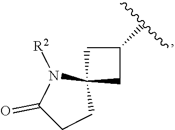

wherein R² is as defined in classes and subclasses herein.

In some embodiments, Cy² is

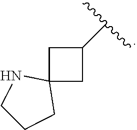 [SMILES: *C1CC2(CCCN2)C1]

In some embodiments, Cy² is

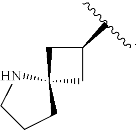 [SMILES: *[C@H]1C[C@@]2(CCCN2)C1]

In some embodiments, Cy² is

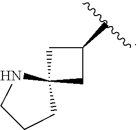 [SMILES: *[C@H]1C[C@]2(CCCN2)C1]

In some embodiments, Cy² is

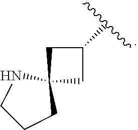 [SMILES: *[C@H]1C[C@]2(CCCN2)C1]

In some embodiments, Cy² is

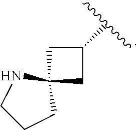 [SMILES: *[C@H]1C[C@@]2(CCCN2)C1]

In some embodiments, Cy² is

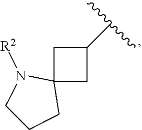

wherein R² is as defined in embodiments and classes and subclasses herein. In some embodiments, Cy² is

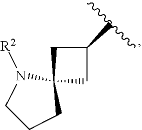

wherein R² is as defined in embodiments and classes and subclasses herein. In some embodiments, Cy² is

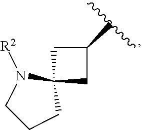

wherein R² is as defined in embodiments and classes and subclasses herein. In some embodiments, Cy² is

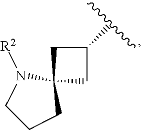

wherein R² is as defined in embodiments and classes and subclasses herein. In some embodiments, Cy² is

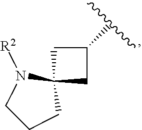

wherein R² is as defined in embodiments and classes and subclasses herein.

In some embodiments, Cy² is

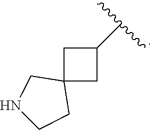 [SMILES: *C1CC2(CCNC2)C1]

In some embodiments, Cy² is

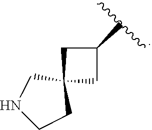 [SMILES: *[C@H]1C[C@@]2(CCNC2)C1]

In some embodiments, Cy² is

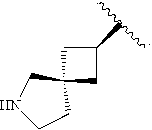 [SMILES: *[C@H]1C[C@]2(CCNC2)C1]

In some embodiments, Cy² is

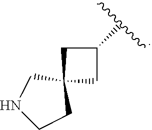 [SMILES: *[C@H]1C[C@]2(CCNC2)C1]

In some embodiments, Cy² is

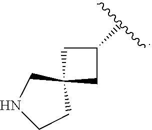 [SMILES: *[C@H]1C[C@@]2(CCNC2)C1]

In some embodiments, Cy² is

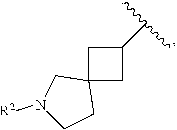

wherein R² is as defined in embodiments and classes and subclasses herein. In some embodiments, Cy² is

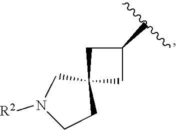

wherein R² is as defined in embodiments and classes and subclasses herein. In some embodiments, Cy² is

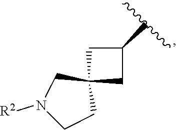

wherein R² is as defined in embodiments and classes and subclasses herein. In some embodiments, Cy² is

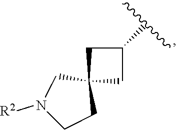

wherein R² is as defined in embodiments and classes and subclasses herein. In some embodiments, Cy² is

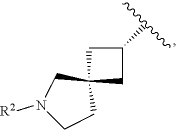

wherein R² is as defined in embodiments and classes and subclasses herein.

In some embodiments, Cy² is

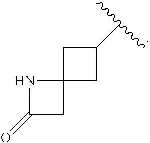 [SMILES: *C1CC2(CC(=O)N2)C1]

In some embodiments, Cy² is

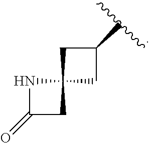 [SMILES: *[C@H]1C[C@@]2(CC(=O)N2)C1]

In some embodiments, Cy² is

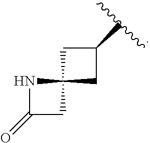 [SMILES: *[C@H]1C[C@]2(CC(=O)N2)C1]

In some embodiments, Cy² is

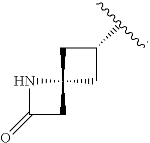 [SMILES: *[C@H]1C[C@]2(CC(=O)N2)C1]

In some embodiments, Cy² is

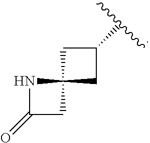 [SMILES: *[C@H]1C[C@@]2(CC(=O)N2)C1]

In some embodiments, Cy² is

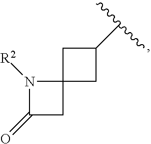

wherein R² is as defined in embodiments and classes and subclasses herein. In some embodiments, Cy² is

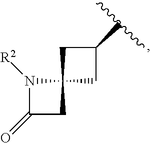

wherein R² is as defined in embodiments and classes and subclasses herein. In some embodiments, Cy² is

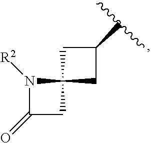

wherein R² is as defined in embodiments and classes and subclasses herein. In some embodiments, Cy² is

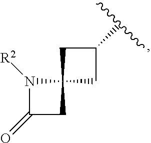

wherein R² is as defined in embodiments and classes and subclasses herein. In some embodiments, Cy² is

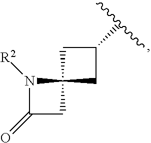

wherein R² is as defined in embodiments and classes and subclasses herein.

In some embodiments, Cy² is

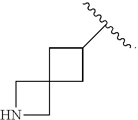 [SMILES: *C1CC2(CNC2)C1]

In some embodiments, Cy² is

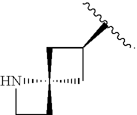 [SMILES: *[C@H]1C[C@@]2(CCN2)C1]

In some embodiments, Cy² is

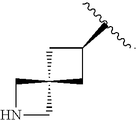

In some embodiments, Cy² is

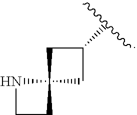 [SMILES: *[C@H]1C[C@]2(CCN2)C1]

In some embodiments, Cy² is

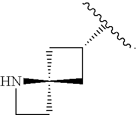 [SMILES: *[C@H]1C[C@@]2(CCN2)C1]

In some embodiments, Cy² is

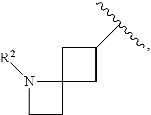

wherein R² is as defined in embodiments and classes and subclasses herein. In some embodiments, Cy² is

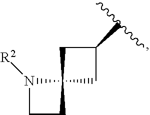

wherein R² is as defined in embodiments and classes and subclasses herein. In some embodiments, Cy² is

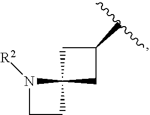

wherein R² is as defined in embodiments and classes and subclasses herein. In some embodiments, Cy² is

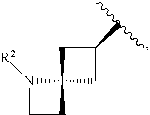

wherein R² is as defined in embodiments and classes and subclasses herein. In some embodiments, Cy² is

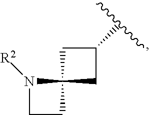

wherein R² is as defined in embodiments and classes and subclasses herein.

In some embodiments, Cy² is

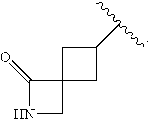 [SMILES: *C1CC2(CNC2=O)C1]

In some embodiments, Cy² is

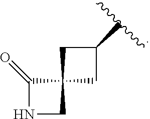 [SMILES: *[C@H]1C[C@@]2(CNC2=O)C1]

In some embodiments, Cy² is

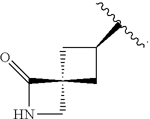 [SMILES: *[C@H]1C[C@]2(CNC2=O)C1]

In some embodiments, Cy² is

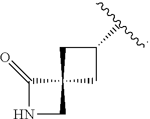 [SMILES: *[C@H]1C[C@]2(CNC2=O)C1]

In some embodiments, Cy² is

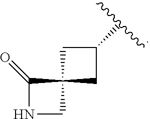 [SMILES: *[C@H]1C[C@@]2(CNC2=O)C1]

In some embodiments, Cy² is

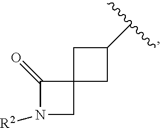

wherein R² is as defined in embodiments and classes and subclasses herein. In some embodiments, Cy² is

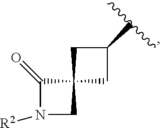

wherein R² is as defined in embodiments and classes and subclasses herein.
In some embodiments, Cy² is

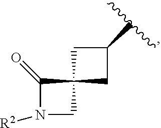

wherein R² is as defined in embodiments and classes and subclasses herein. In some embodiments, Cy² is

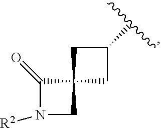

wherein R² is as defined in embodiments and classes and subclasses herein. In some embodiments, Cy² is

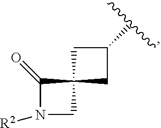

wherein R² is as defined in embodiments and classes and subclasses herein.

In some embodiments, Cy² is

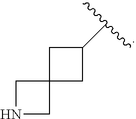 [SMILES: *C1CC2(CNC2)C1]

In some embodiments, Cy² is

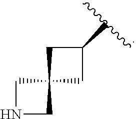

In some embodiments, Cy² is

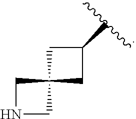

In some embodiments, Cy² is

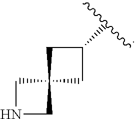

In some embodiments, Cy² is

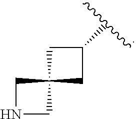

In some embodiments, Cy² is

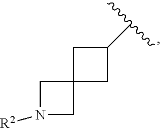

wherein R² is as defined in embodiments and classes and subclasses herein. In some embodiments, Cy² is

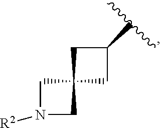

wherein R² is as defined in embodiments and classes and subclasses herein. In some embodiments, Cy² is

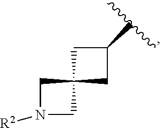

wherein R² is as defined in embodiments and classes and subclasses herein. In some embodiments, Cy² is

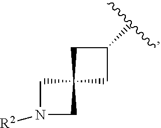

wherein R² is as defined in embodiments and classes and subclasses herein. In some embodiments, Cy² is

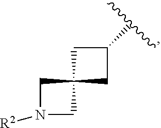

wherein R² is as defined in embodiments and classes and subclasses herein.

In some embodiments, Cy² is a 5-6 membered monocyclic heteroaryl ring having 1-4 heteroatoms independently selected from nitrogen, oxygen, and sulfur, wherein Cy² is substituted with m instances of R². In some embodiments, Cy² is a 5-6 membered monocyclic heteroaryl ring having 1-2 heteroatoms independently selected from nitrogen, oxygen, and sulfur, wherein Cy² is substituted with m instances of R². In some embodiments, Cy² is pyridyl, pyrimidinyl, pyridazinyl, triazinyl, or tetrazinyl. In some embodiments, Cy² is

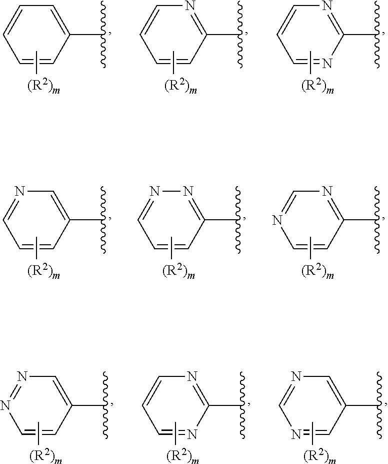

wherein each R² is as defined in embodiments and classes and subclasses herein. In some embodiments, Cy² is

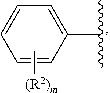

wherein each R² is as defined in embodiments and classes and subclasses herein. In some embodiments, Cy² is

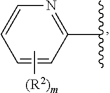

wherein each R² is as defined in embodiments and classes and subclasses herein. In some embodiments, Cy² is

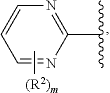

wherein each R² is as defined in embodiments and classes and subclasses herein. In some embodiments, Cy² is

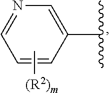

wherein each R² is as defined in embodiments and classes and subclasses herein. In some embodiments, Cy² is

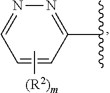

wherein each R² is as defined in embodiments and classes and subclasses herein. In some embodiments, Cy² is

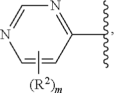

wherein each R² is as defined in embodiments and classes and subclasses herein. In some embodiments, Cy² is

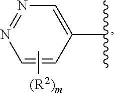

wherein each R² is as defined in embodiments and classes and subclasses herein. In some embodiments, Cy² is

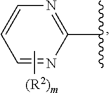

wherein each R² is as defined in embodiments and classes and subclasses herein. In some embodiments, Cy² is

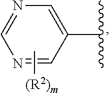

wherein each R² is as defined in embodiments and classes and subclasses herein.

In some embodiments, Cy² is a 3-7 membered saturated or partially unsaturated monocyclic heterocyclic ring having 1-2 heteroatoms independently selected from nitrogen, oxygen, and sulfur; wherein Cy² is substituted with m instances of R². In some embodiments, Cy² is aziridinyl, oxiranyl, azetidinyl, oxetanyl, pyrrolidinyl, piperidinyl, piperazinyl, tetrahydrofuranyl, tetrahydropyranyl, dioxanyl, morpholinyl, tetrahydrothiofuranyl, tetrahydrothiopyranyl, thiomorpholinyl, azepanyl, homomorpholinyl, and homothiomorpholinyl. In some embodiments, Cy² is azetidinyl, pyrrolidinyl or piperidinyl. In some embodiments, Cy² is

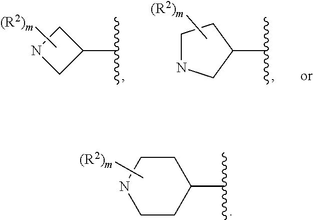

In some embodiments, Cy² is selected from the groups depicted in the compounds in Table 1.

As defined generally above, Q is L^{Q}, wherein L^{Q} is as defined in embodiments and classes and subclasses herein.

In some embodiments, Q is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, Q is a covalent bond. In some embodiments, Q is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, Q is a C₁₋₄ bivalent saturated or unsaturated, straight, or branched hydrocarbon chain.

In some embodiments, Q is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, Q is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, Q is a C₁₋₂ bivalent saturated or unsaturated hydrocarbon chain.

In some embodiments, Q is —C(O)N(R)—, —C(O)N(R)CH₂—, —N(R)—, —CH₂C(O)N(R)—, —N(R)C(O)N(R)—, or a covalent bond. In some embodiments, Q is —C(O)N(H)—, —C(O)N(H)CH₂—, —N(H)—, —CH₂C(O)N(H)—, —N(H)C(O)N(H)—, or a covalent bond. In some embodiments, Q is —C(O)N(H)—, —C(O)N(H)CH₂—, or a covalent bond. In some embodiments, Q is —C(O)N(H)— or —C(O)N(H)CH₂—. In some embodiments, Q is —C(O)N(H)—. In some embodiments, Q is —C(O)N(H)CH₂—. In some embodiments, Q is —N(H)—. In some embodiments, Q is —CH₂C(O)N(H)—. In some embodiments, Q is —N(H)C(O)N(H)—. In some embodiments, Q is a covalent bond.

In some embodiments, Q is selected from the groups depicted in the compounds in Table 1.

As defined generally above, T is a bivalent C₁₋₃ aliphatic chain substituted with q instances of R^{T}. In some embodiments, T is a bivalent C₂₋₃ aliphatic chain substituted with q instances of R^{T}. In some embodiments, T is a bivalent C₁₂ aliphatic chain substituted with q instances of R^{T}. In some embodiments, T is a bivalent C₁ aliphatic chain substituted with q instances of R^{T}. In some embodiments, T is a bivalent C₂ aliphatic chain substituted with q instances of R^{T}. In some embodiments, T is a bivalent C₃ aliphatic chain substituted with q instances of R^{T}.

In some embodiments, T is

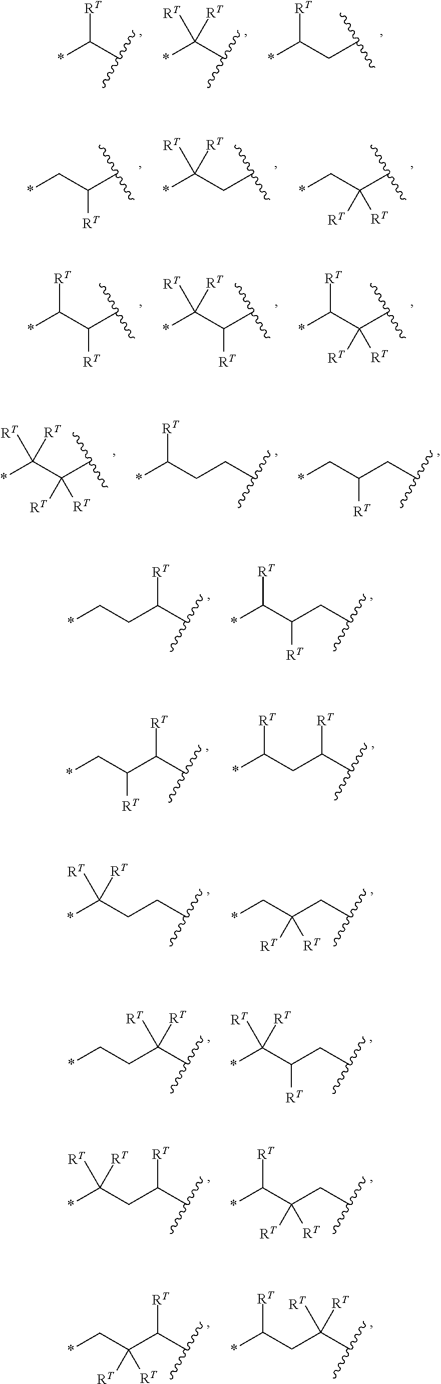

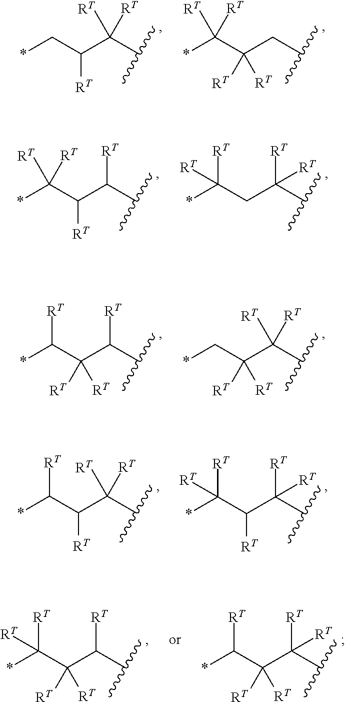

wherein [symbol] represents a bond to Q and [symbol] represents a bond to Cy¹, and wherein R^{T} is defined in embodiments and classes and subclasses herein.

In some embodiments, T is

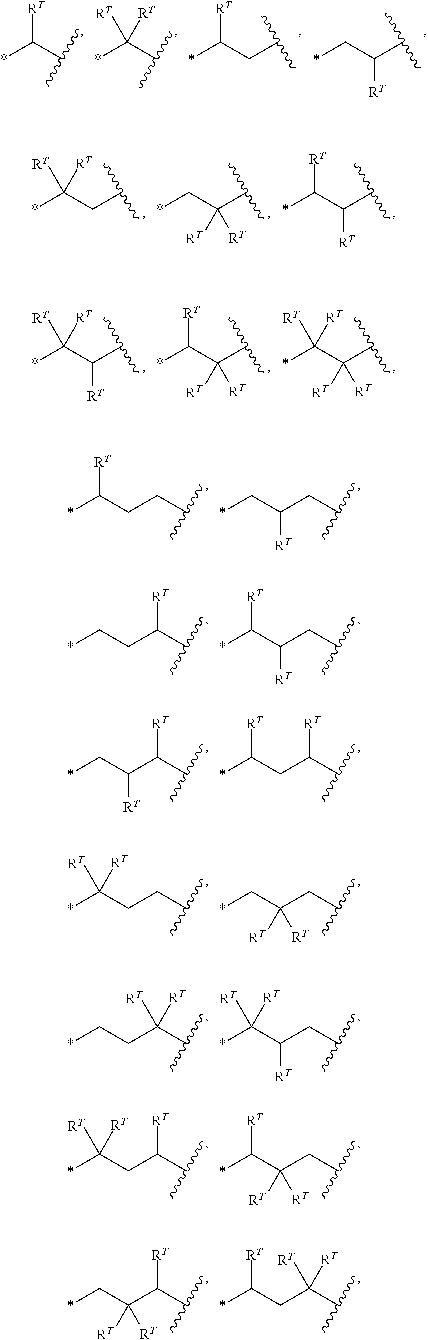

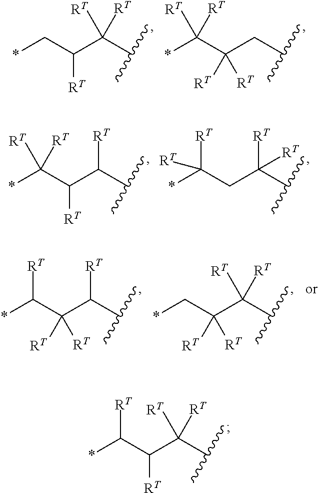

wherein [symbol] represents a bond to Q and [symbol] represents a bond to Cy¹, and wherein R^{T} is as defined in embodiments and classes and subclasses herein.

In some embodiments, T is

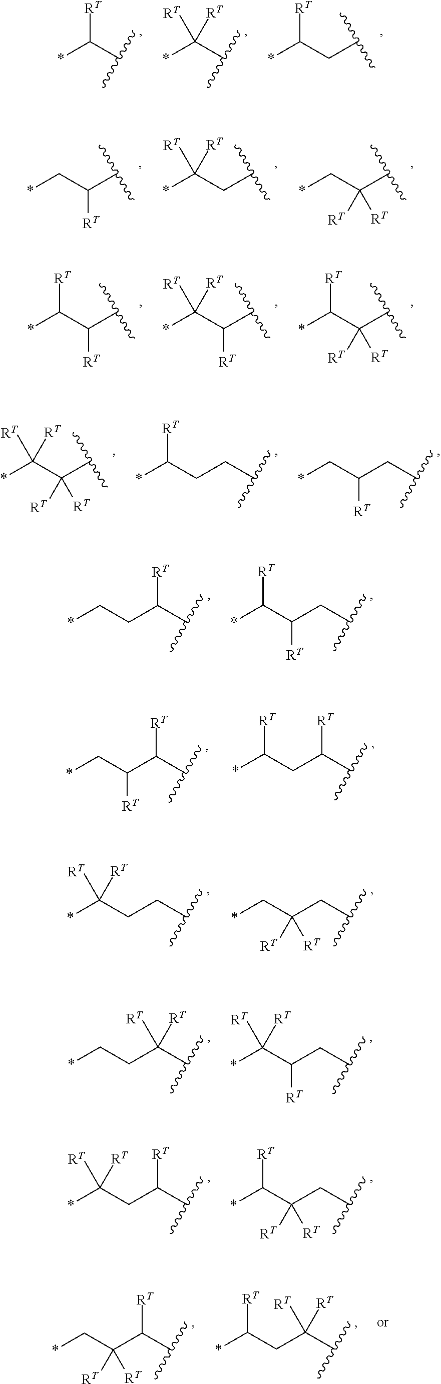

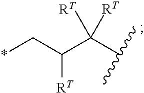

wherein [symbol] represents a bond to Q and [symbol] represents a bond to Cy¹ and wherein R^{T} is as defined in embodiments and classes and subclasses herein.

In some embodiments, T is

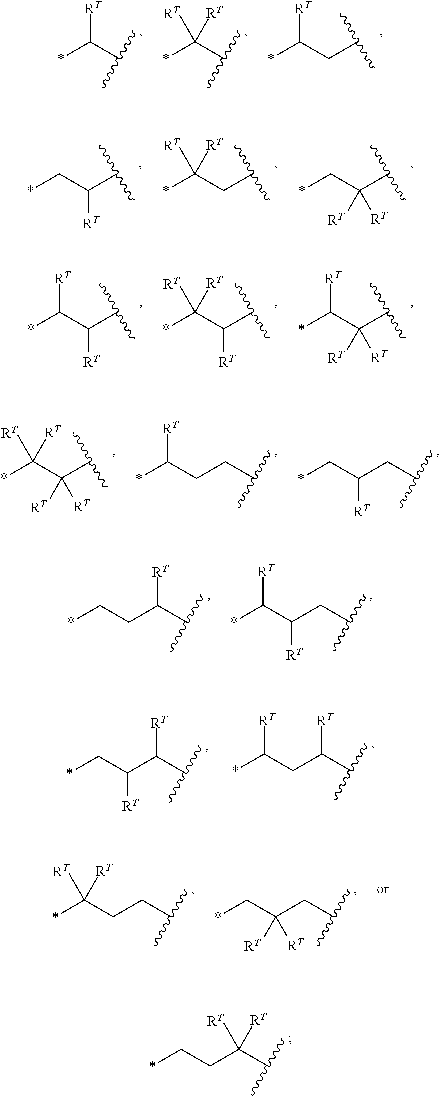

wherein [symbol] represents a bond to Q and [symbol] represents a bond to Cy¹, and wherein R^{T} is as defined in embodiments and classes and subclasses herein.

In some embodiments, T is

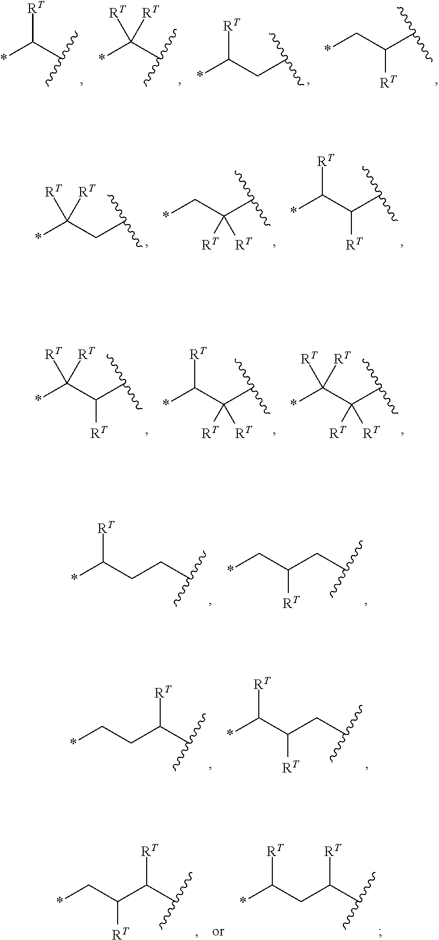

wherein [symbol] represents a bond to Q and [symbol] represents a bond to Cy¹, and wherein R^{T} is as defined in embodiments and classes and subclasses herein.

In some embodiments, T is

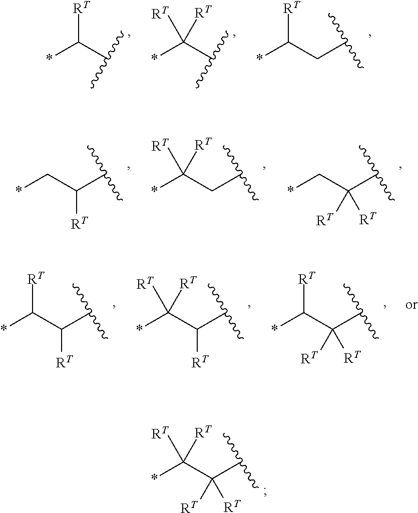

wherein [symbol] represents a bond to Q and [symbol] represents a bond to Cy¹, and wherein R^{T} is as defined in embodiments and classes and subclasses herein.

In some embodiments, T is

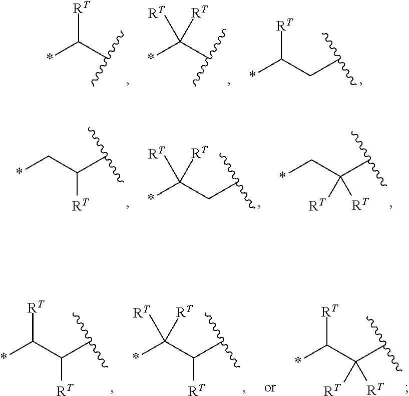

wherein [symbol] represents a bond to Q and [symbol] represents a bond to Cy¹, and wherein R^{T} is as defined in embodiments and classes and subclasses herein.

In some embodiments, T is

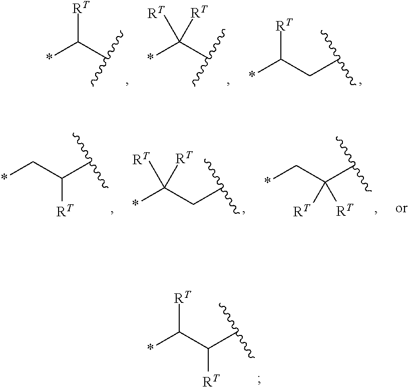

wherein [symbol] represents a bond to Q and [symbol] represents a bond to Cy¹, and wherein R^{T} is as defined in embodiments and classes and subclasses herein.

In some embodiments, T is

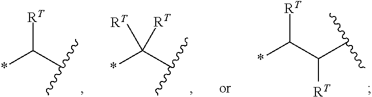

wherein [symbol] represents a bond to Q and [symbol] represents a bond to Cy¹, and wherein R^{T} is as defined in embodiments and classes and subclasses herein.

In some embodiments, T is

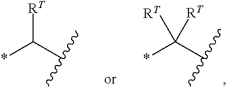

wherein [symbol] represents a bond to Q and [symbol] represents a bond to Cy¹, and wherein R^{T} is as defined in embodiments and classes and subclasses herein.

In some embodiments, T is

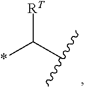

wherein [symbol] represents a bond to Q and [symbol] represents a bond to Cy¹, and wherein R^{T} is as defined in embodiments and classes and subclasses herein. In some embodiments, T is

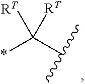

wherein [symbol] represents a bond to Q and [symbol] represents a bond to Cy¹, and wherein R^{T} is as defined in embodiments and classes and subclasses herein. In some embodiments, T is

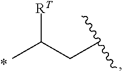

wherein [symbol] represents a bond to Q and [symbol] represents a bond to Cy¹, and wherein R^{T} is as defined in embodiments and classes and subclasses herein. In some embodiments, T is

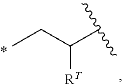

wherein [symbol] represents a bond to Q and [symbol] represents a bond to Cy¹, and wherein R^{T} is as defined in embodiments and classes and subclasses herein. In some embodiments, T is

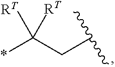

wherein [symbol] represents a bond to Q and [symbol] represents a bond to Cy¹, and wherein R^{T} is as defined in embodiments and classes and subclasses herein. In some embodiments, T is

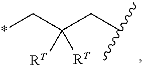

wherein [symbol] represents a bond to Q and [symbol] represents a bond to Cy¹, and wherein R^{T} is as defined in embodiments and classes and subclasses herein. In some embodiments, T is

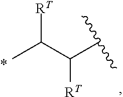

wherein [symbol] represents a bond to Q and [symbol] represents a bond to Cy¹, and wherein R^{T} is as defined in embodiments and classes and subclasses herein. In some embodiments, T is

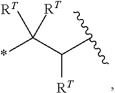

wherein [symbol] represents a bond to Q and [symbol] represents a bond to Cy¹, and wherein R^{T} is as defined in embodiments and classes and subclasses herein. In some embodiments, T is

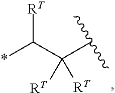

wherein [symbol] represents a bond to Q and [symbol] represents a bond to Cy¹, and wherein R^{T} is as defined in embodiments and classes and subclasses herein. In some embodiments, T is

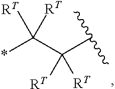

wherein [symbol] represents a bond to Q and [symbol] represents a bond to Cy¹, and wherein R^{T} is as defined in embodiments and classes and subclasses herein. In some embodiments, T is

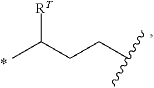

wherein [symbol] represents a bond to Q and [symbol] represents a bond to Cy¹, and wherein R^{T} is as defined in embodiments and classes and subclasses herein. In some embodiments, T is

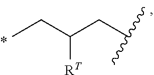

wherein [symbol] represents a bond to Q and [symbol] represents a bond to Cy¹, and wherein R^{T} is as defined in embodiments and classes and subclasses herein. In some embodiments, T is

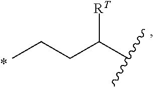

wherein [symbol] represents a bond to Q and [symbol] represents a bond to Cy¹, and wherein R¹ is as defined in embodiments and classes and subclasses herein. In some embodiments, T is

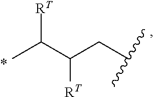

wherein [symbol] represents a bond to Q and [symbol] represents a bond to Cy¹, and wherein R^{T} is as defined in embodiments and classes and subclasses herein. In some embodiments, T is

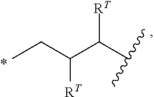

wherein [symbol] represents a bond to Q and [symbol] represents a bond to Cy¹, and wherein R^{T} is as defined in embodiments and classes and subclasses herein. In some embodiments, T is

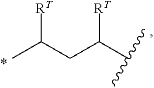

wherein [symbol] represents a bond to Q and [symbol] represents a bond to Cy¹, and wherein R^{T} is as defined in embodiments and classes and subclasses herein. In some embodiments, T is

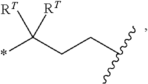

wherein [symbol] represents a bond to Q and [symbol] represents a bond to Cy¹, and wherein R^{T} is as defined in embodiments and classes and subclasses herein. In some embodiments, T is

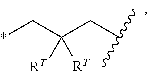

wherein [symbol] represents a bond to Q and [symbol] represents a bond to Cy¹, and wherein R^{T} is as defined in embodiments and classes and subclasses herein. In some embodiments, T is,

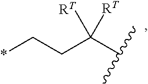

wherein [symbol] represents a bond to Q and [symbol] represents a bond to Cy¹, and wherein R^{T} is as defined in embodiments and classes and subclasses herein. In some embodiments, T is

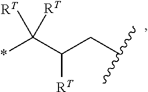

wherein [symbol] represents a bond to Q and [symbol] represents a bond to Cy¹, and wherein R^{T} is as defined in embodiments and classes and subclasses herein. In some embodiments, T is

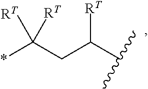

wherein [symbol] represents a bond to Q and [symbol] represents a bond to Cy¹, and wherein R^{T} is as defined in embodiments and classes and subclasses herein. In some embodiments, T is

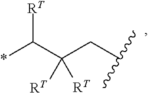

wherein [symbol] represents a bond to Q and [symbol] represents a bond to Cy¹, and wherein R^{T} is as defined in embodiments and classes and subclasses herein. In some embodiments, T is

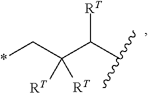

wherein [symbol] represents a bond to Q and [symbol] represents a bond to Cy¹, and wherein R^{T} is as defined in embodiments and classes and subclasses herein. In some embodiments, T is

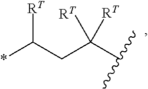

wherein [symbol] represents a bond to Q and [symbol] represents a bond to Cy¹, and wherein R^{T} is as defined in embodiments and classes and subclasses herein. In some embodiments, T is

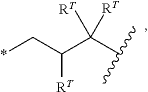

wherein [symbol] represents a bond to Q and [symbol] represents a bond to Cy¹, and wherein R^{T} is as defined in embodiments and classes and subclasses herein. In some embodiments, T is

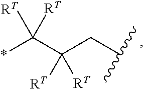

wherein [symbol] πrepresents a bond to Q and [symbol] represents a bond to Cy¹, and wherein R^{T} is as defined in embodiments and classes and subclasses herein. In some embodiments, T is

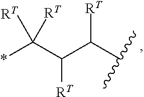

wherein [symbol] represents a bond to Q and [symbol] represents a bond to Cy¹, and wherein R^{T} is as defined in embodiments and classes and subclasses herein. In some embodiments, T is

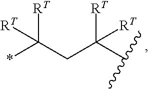

wherein [symbol] represents a bond to Q and [symbol] represents a bond to Cy¹, and wherein R^{T} is as defined in embodiments and classes and subclasses herein. In some embodiments, T is

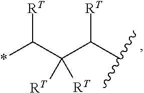

wherein [symbol] represents a bond to Q and [symbol] represents a bond to Cy¹, and wherein R^{T} is as defined in embodiments and classes and subclasses herein. In some embodiments, T is

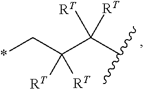

wherein [symbol] represents a bond to Q and [symbol] represents a bond to Cy¹, and wherein R^{T} is as defined in embodiments and classes and subclasses herein. In some embodiments, T is

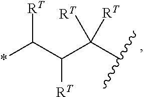

wherein [symbol] represents a bond to Q and [symbol] represents a bond to Cy¹, and wherein R^{T} is as defined in embodiments and classes and subclasses herein. In some embodiments, T is

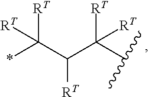

wherein [symbol] represents a bond to Q and [symbol] represents a bond to Cy¹, and wherein R^{T} is as defined in embodiments and classes and subclasses herein. In some embodiments, T is

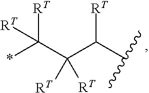

wherein [symbol] represents a bond to Q and [symbol] represents a bond to Cy¹, and wherein R^{T} is as defined in embodiments and classes and subclasses herein. In some embodiments, T is

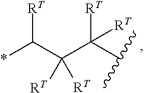

wherein [symbol] represents a bond to Q and [symbol] represents a bond to Cy¹, and wherein R^{T} is as defined in embodiments and classes and subclasses herein.

In some embodiments, T is

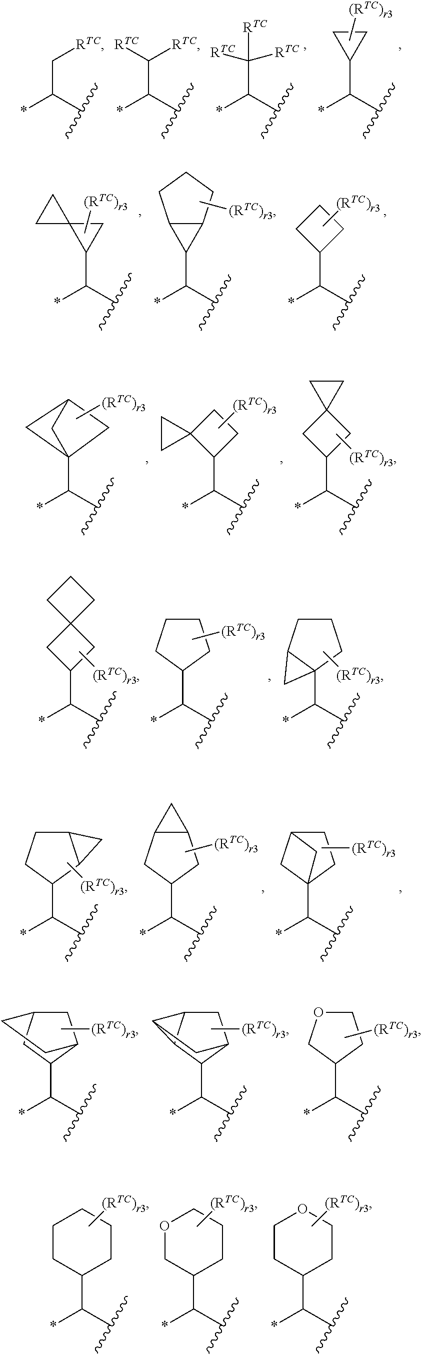

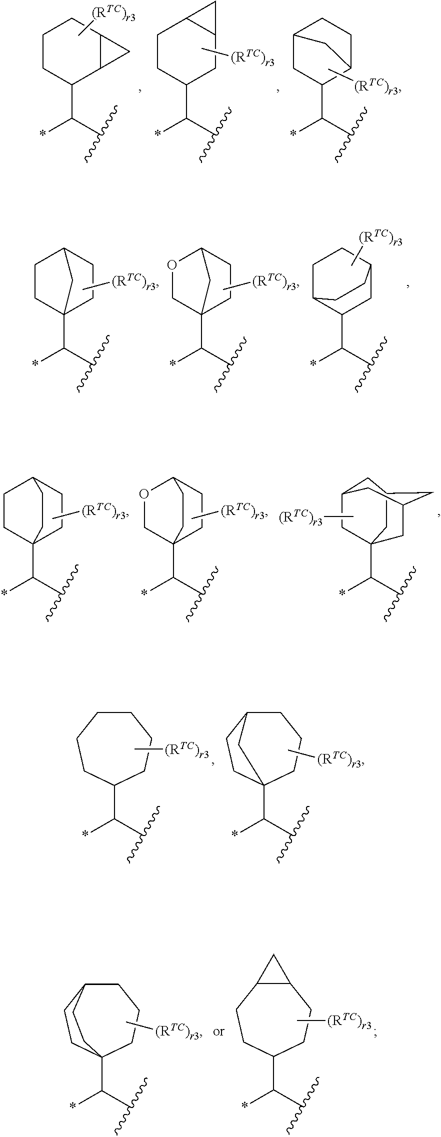

wherein [symbol] represents a bond to Q and [symbol] represents a bond to Cy¹, and wherein R^{TC} and r³ are as defined in embodiments and classes and subclasses herein.

In some embodiments, T is

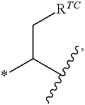

wherein [symbol] represents a bond to Q and [symbol] represents a bond to Cy¹, and wherein R^{TC} is as defined in embodiments and classes and subclasses herein. In some embodiments, T is

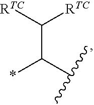

wherein [symbol] represents a bond to Q and [symbol] represents a bond to Cy¹, and wherein R^{TC} is as defined in embodiments and classes and subclasses herein. In some embodiments, T is

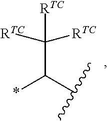

wherein [symbol] represents a bond to Q and [symbol] represents a bond to Cy¹, and wherein R^{TC} is as defined in embodiments and classes and subclasses herein. In some embodiments, T is

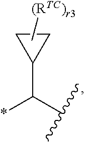

wherein [symbol] represents a bond to Q and [symbol] represents a bond to Cy¹, and wherein R^{TC} and r³ are as defined in embodiments and classes and subclasses herein. In some embodiments, T is

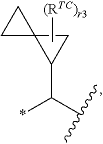

wherein [symbol] represents a bond to Q and [symbol] represents a bond to Cy¹, and wherein R^{TC} and r³ are as defined in embodiments and classes and subclasses herein. In some embodiments, T is

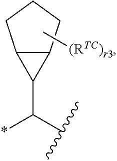

wherein [symbol] represents a bond to Q and [symbol] represents a bond to Cy¹, and wherein R^{TC} and r³ are as defined in embodiments and classes and subclasses herein. In some embodiments, T is

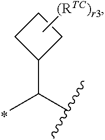

wherein [symbol] represents a bond to Q and [symbol] represents a bond to Cy¹, and wherein R^{TC} and r³ are as defined in embodiments and classes and subclasses herein. In some embodiments, T is

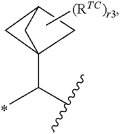

wherein [symbol] represents a bond to Q and [symbol] represents a bond to Cy¹, and wherein R^{TC} and r³ are as defined in embodiments and classes and subclasses herein. In some embodiments, T is,

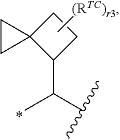

wherein [symbol] represents a bond to Q and [symbol] represents a bond to Cy¹, and wherein R^{TC} and r³ are as defined in embodiments and classes and subclasses herein. In some embodiments, T is

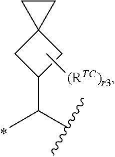

wherein [symbol] represents a bond to Q and [symbol] represents a bond to Cy¹, and wherein R^{TC} and r³ are as defined in embodiments and classes and subclasses herein. In some embodiments, T is

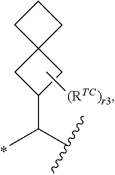

wherein [symbol] represents a bond to Q and [symbol] represents a bond to Cy¹, and wherein R^{TC} and r³ are as defined in embodiments and classes and subclasses herein. In some embodiments, T is

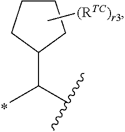

wherein [symbol] represents a bond to Q and [symbol] represents a bond to Cy¹, and wherein R^{TC} and r³ are as defined in embodiments and classes and subclasses herein. In some embodiments, T is

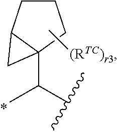

wherein [symbol] represents a bond to Q and [symbol] represents a bond to Cy¹, and wherein R^{TC} and r³ are as defined in embodiments and classes and subclasses herein. In some embodiments, T is

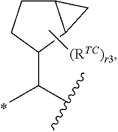

wherein [symbol] represents a bond to Q and [symbol] represents a bond to Cy¹, and wherein R^{TC} and r³ are as defined in embodiments and classes and subclasses herein. In some embodiments, T is

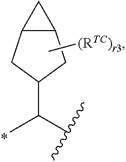

wherein [symbol] represents a bond to Q and [symbol] represents a bond to Cy¹, and wherein R^{TC} and r³ are as defined in embodiments and classes and subclasses herein. In some embodiments, T is

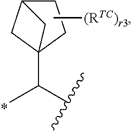

wherein [symbol] represents a bond to Q and [symbol] represents a bond to Cy¹, and wherein R^{TC} and r³ are as defined in embodiments and classes and subclasses herein. In some embodiments, T is

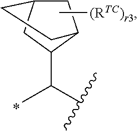

wherein [symbol] represents a bond to Q and [symbol] represents a bond to Cy¹, and wherein R^{TC} and r³ are as defined in embodiments and classes and subclasses herein. In some embodiments, T is

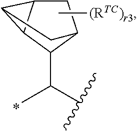

wherein [symbol] represents a bond to Q and [symbol] represents a bond to Cy and wherein R^{TC} and r³ are as defined in embodiments and classes and subclasses herein. In some embodiments, T is

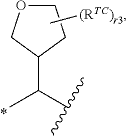

wherein [symbol] represents a bond to Q and [symbol] represents a bond to Cy¹, and wherein R^{TC} and r³ are as defined in embodiments and classes and subclasses herein. In some embodiments, T is

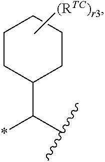

wherein [symbol] represents a bond to Q and [symbol] represents a bond to Cy¹, and wherein R^{TC} and r³ are as defined in embodiments and classes and subclasses herein. In some embodiments, T is

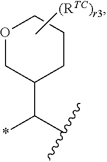

wherein [symbol] represents a bond to Q and [symbol] represents a bond to Cy¹, and wherein R^{TC} and r³ are as defined in embodiments and classes and subclasses herein. In some embodiments, T is

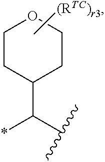

wherein [symbol] represents a bond to Q and [symbol] represents a bond to Cy¹, and wherein R^{TC} and r³ are as defined in embodiments and classes and subclasses herein. In some embodiments, T is

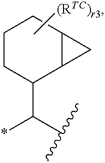

wherein [symbol] represents a bond to Q and [symbol] represents a bond to Cy¹, and wherein R^{TC} and r³ are as defined in embodiments and classes and subclasses herein. In some embodiments, T is

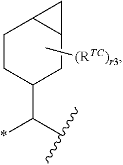

wherein [symbol] represents a bond to Q and [symbol] represents a bond to Cy¹, and wherein R^{TC} and r³ are as defined in embodiments and classes and subclasses herein. In some embodiments, T is

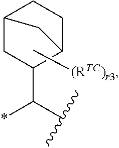

wherein [symbol] represents a bond to Q and [symbol] represents a bond to Cy¹, and wherein R^{TC} and r³ are as defined in embodiments and classes and subclasses herein. In some embodiments, T is

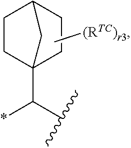

wherein [symbol] represents a bond to Q and [symbol] represents a bond to Cy¹, and wherein R^{TC} and r³ are as defined in embodiments and classes and subclasses herein. In some embodiments, T is

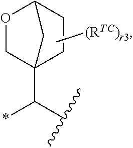

wherein [symbol] represents a bond to Q and [symbol] represents a bond to Cy¹, and wherein R^{TC} and r³ are as defined in embodiments and classes and subclasses herein. In some embodiments, T is

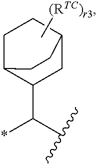

wherein [symbol] represents a bond to Q and [symbol] represents a bond to Cy¹, and wherein R^{TC} and r³ are as defined in embodiments and classes and subclasses herein. In some embodiments, T is

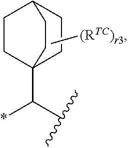

wherein [symbol] represents a bond to Q and [symbol] represents a bond to Cy¹, and wherein R^{TC} and r³ are as defined in embodiments and classes and subclasses herein. In some embodiments, T is

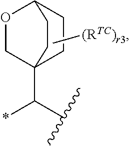

wherein [symbol] represents a bond to Q and [symbol] represents a bond to Cy¹, and wherein R^{TC} and r³ are as defined in embodiments and classes and subclasses herein. In some embodiments, T is

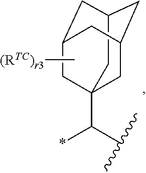

wherein [symbol] represents a bond to Q and [symbol] represents a bond to Cy¹, and wherein R^{TC} and r³ are as defined in embodiments and classes and subclasses herein. In some embodiments, T is

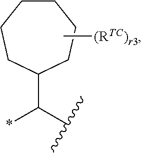

wherein [symbol] represents a bond to Q and [symbol] represents a bond to Cy¹, and wherein R^{TC} and r³ are as defined in embodiments and classes and subclasses herein. In some embodiments, T is

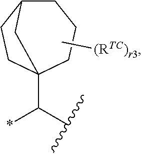

wherein [symbol] represents a bond to Q and [symbol] represents a bond to Cy¹, and wherein R^{TC} and r³ are as defined in embodiments and classes and subclasses herein. In some embodiments, T is

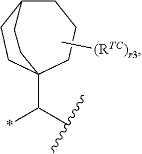

wherein [symbol] represents a bond to Q and [symbol] represents a bond to Cy¹, and wherein R^{TC} and r³ are as defined in embodiments and classes and subclasses herein. In some embodiments, T is

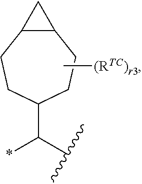

wherein [symbol] represents a bond to Q and [symbol] represents a bond to Cy¹, and wherein R^{TC} and r³ are as defined in embodiments and classes and subclasses herein.

In some embodiments, T is

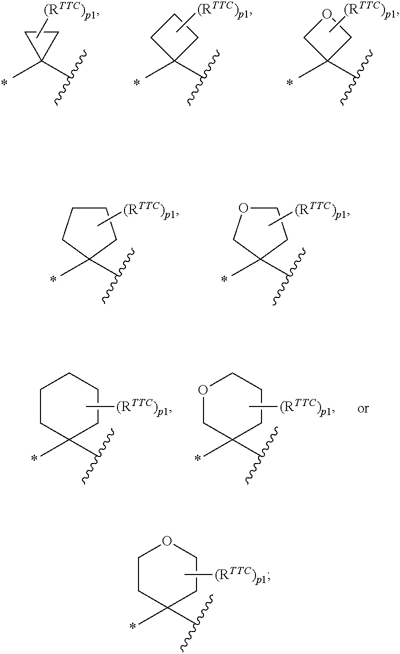

wherein [symbol] represents a bond to Q and [symbol] represents a bond to Cy¹, and wherein R^{TTC} and p¹ are as defined in embodiments and classes and subclasses herein.

In some embodiments, T is

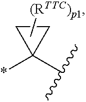

wherein [symbol] represents a bond to Q and [symbol] represents a bond to Cy¹, and wherein R^{TTC} and p¹ are as defined in embodiments and classes and subclasses herein. In some embodiments, T is

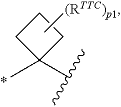

wherein represents a bond to Q and [symbol] represents a bond to Cy¹, and wherein R^{TTC} and p¹ are as defined in embodiments and classes and subclasses herein. In some embodiments, T is

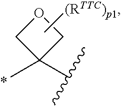

wherein [symbol] represents a bond to Q and [symbol] represents a bond to Cy¹, and wherein R^{TTC} and p¹ are as defined in embodiments and classes and subclasses herein. In some embodiments, T is

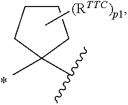

wherein [symbol] represents a bond to Q and [symbol] represents a bond to Cy¹, and wherein R^{TTC} and p¹ are as defined in embodiments and classes and subclasses herein. In some embodiments, T is

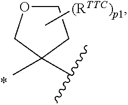

wherein [symbol] represents a bond to Q and [symbol] represents a bond to Cy¹, and wherein R^{TTC} and p¹ are as defined in embodiments and classes and subclasses herein. In some embodiments, T is

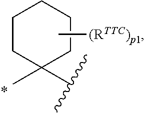

wherein [symbol] represents a bond to Q and [symbol] represents a bond to Cy¹, and wherein R^{TTC} and p¹ are as defined in embodiments and classes and subclasses herein. In some embodiments, T is

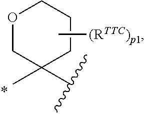

wherein [symbol] represents a bond to Q and [symbol] represents a bond to Cy¹, and wherein R^{TTC} and p¹ are as defined in embodiments and classes and subclasses herein. In some embodiments, T is

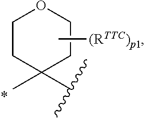

wherein [symbol] represents a bond to Q and [symbol] represents a bond to Cy¹, and wherein R^{TTC} and p¹ are as defined in embodiments and classes and subclasses herein.

In some embodiments, T is

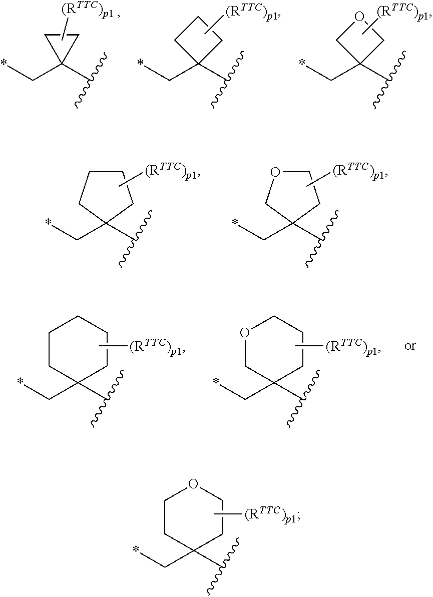

wherein [symbol] represents a bond to Q and [symbol] represents a bond to Cy¹, and wherein R^{TTC} and p¹ are as defined in embodiments and classes and subclasses herein.

In some embodiments, T is

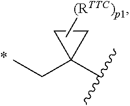

wherein [symbol] represents a bond to Q and [symbol] represents a bond to Cy¹, and wherein R^{TTC} and p¹ are as defined in embodiments and classes and subclasses herein. In some embodiments, T is

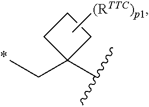

wherein [symbol] represents a bond to Q and [symbol] represents a bond to Cy¹, and wherein R^{TTC} and p¹ are as defined in embodiments and classes and subclasses herein. In some embodiments, T is

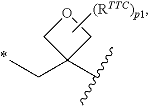

wherein [symbol] represents a bond to Q and [symbol] represents a bond to Cy¹, and wherein R^{TTC} and p¹ are as defined in embodiments and classes and subclasses herein. In some embodiments, T is

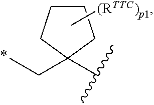

wherein [symbol] represents a bond to Q and [symbol] represents a bond to Cy¹, and wherein R^{TTC} and p¹ are as defined in embodiments and classes and subclasses herein. In some embodiments, T is

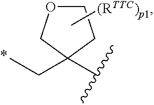

wherein [symbol] represents a bond to Q and [symbol] represents a bond to Cy¹, and wherein R^{TTC} and p¹ are as defined in embodiments and classes and subclasses herein. In some embodiments, T is

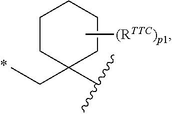

wherein [symbol] represents a bond to Q and [symbol] represents a bond to Cy¹, and wherein R^{TTC} and p¹ are as defined in embodiments and classes and subclasses herein. In some embodiments, T is

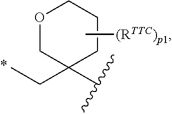

wherein [symbol] represents a bond to Q and [symbol] represents a bond to Cy¹, and wherein R^{TTC} and p¹ are as defined in embodiments and classes and subclasses herein. In some embodiments, T is

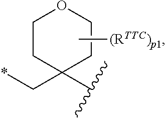

wherein [symbol] represents a bond to Q and [symbol] represents a bond to Cy¹, and wherein R^{TTC} and p¹ are as defined in embodiments and classes and subclasses herein.

In some embodiments, T is or

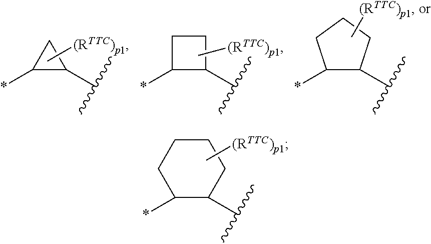

wherein [symbol] represents a bond to Q and [symbol] represents a bond to Cy¹, and wherein R^{TTC} and p¹ are as defined in embodiments and classes and subclasses herein.

In some embodiments, T is

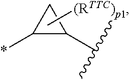

wherein [symbol] represents a bond to Q and [symbol] represents a bond to Cy¹, and wherein R^{TTC} and p¹ are as defined in embodiments and classes and subclasses herein. In some embodiments, T is

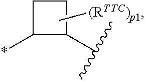

wherein [symbol] represents a bond to Q and [symbol] represents a bond to Cy¹, and wherein R^{TTC} and p¹ are as defined in embodiments and classes and subclasses herein. In some embodiments, T is

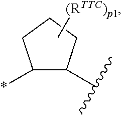

wherein [symbol] represents a bond to Q and [symbol] represents a bond to Cy¹, and wherein R^{TTC} and p¹ are as defined in embodiments and classes and subclasses herein. In some embodiments, T is

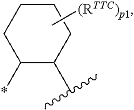

wherein [symbol] represents a bond to Q and [symbol] represents a bond to Cy¹, and wherein R^{TTC} and p¹ are as defined in embodiments and classes and subclasses herein. In some embodiments, T is

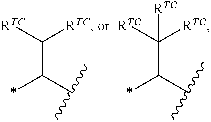

wherein each R^{TC} is as defined in embodiments and classes and subclasses herein. In some embodiments, T is

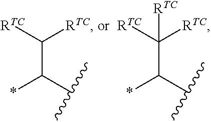

wherein each R^{TC} is as defined in embodiments and classes and subclasses herein. In some embodiments, T is

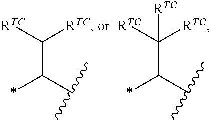

wherein each R^{TC} is independently deuterium, halogen, or an optionally substituted group selected from C₁₋₆ aliphatic. In some embodiments, T is

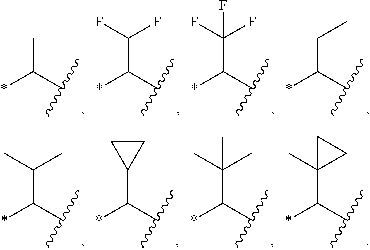

In some embodiments, T is

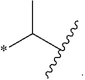 [SMILES: *C(*)C]

In some embodiments. T is

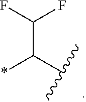 [SMILES: *C(*)C(F)F]

In some embodiments, T is

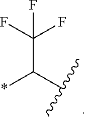 [SMILES: *C(*)C(F)(F)F]

In some embodiments, T is

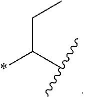 [SMILES: *C(*)CC]

In some embodiments, T is

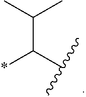 [SMILES: *C(*)C(C)C]

In some embodiments, T is

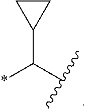 [SMILES: *C(*)C1CC1]

In some embodiments, T is

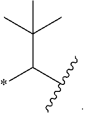 [SMILES: *C(*)C(C)(C)C]

In some embodiments, T is

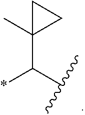 [SMILES: *C(*)C1(C)CC1]

In some embodiments, T is selected from the groups depicted in the compounds in Table 1.

As defined generally above, each R¹ is independently -L¹-R^{1A}; or two instances of R¹ are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p² instances of R^{11C}; or one instance of R^{T} and one instance of R¹ are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p⁴ instances of R^{T1C}.

In some embodiments, each R¹ is independently -L¹-R^{1A}. In some embodiments, each R¹ is independently —R^{1A}.

In some embodiments, each R¹ is independently R^{A}. In some embodiments, each R¹ (i.e., -L¹-R^{1A} taken together) is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂; wherein R is as defined in embodiments and classes and subclasses herein.

In some embodiments, R¹ is oxo. In some embodiments, R¹ is deuterium. In some embodiments, each R¹ is independently halogen. In some embodiments, R¹ is —CN. In some embodiments, R¹ is —NO₂. In some embodiments, each R¹ is independently —OR, wherein R is as defined in embodiments and classes and subclasses herein. In some embodiments, R¹ is —SF₅. In some embodiments, each R¹ is independently —SR, wherein R is as defined in embodiments and classes and subclasses herein. In some embodiments, each R¹ is independently —NR₂, wherein R is as defined in embodiments and classes and subclasses herein. In some embodiments, each R¹ is independently —S(O)₂R, wherein R is as defined in embodiments and classes and subclasses herein. In some embodiments, each R¹ is independently —S(O)₂NR₂, wherein R is as defined in embodiments and classes and subclasses herein. In some embodiments, R¹ is —S(O)₂F. In some embodiments, each R¹ is independently —S(O)R, wherein R is as defined in embodiments and classes and subclasses herein. In some embodiments, each R¹ is independently —S(O)NR₂, wherein R is as defined in embodiments and classes and subclasses herein. In some embodiments, each R¹ is independently —S(O)(NR)R, wherein R is as defined in embodiments and classes and subclasses herein. In some embodiments, each R¹ is independently —S(O)(NCN)R, wherein R is as defined in embodiments and classes and subclasses herein. In some embodiments, each R¹ is independently —S(NCN)R, wherein R is as defined in embodiments and classes and subclasses herein. In some embodiments, each R¹ is independently —C(O)R, wherein R is as defined in embodiments and classes and subclasses herein. In some embodiments, each R¹ is independently —C(O)OR, wherein R is as defined in embodiments and classes and subclasses herein. In some embodiments, each R¹ is independently —C(O)NR₂, wherein R is as defined in embodiments and classes and subclasses herein. In some embodiments, each R¹ is independently —C(O)N(R)OR, wherein R is as defined in embodiments and classes and subclasses herein. In some embodiments, each R¹ is independently —OC(O)R, wherein R is as defined in embodiments and classes and subclasses herein. In some embodiments, each R¹ is independently —OC(O)NR₂, wherein R is as defined in embodiments and classes and subclasses herein. In some embodiments, each R¹ is independently —N(R)C(O)OR, wherein R is as defined in embodiments and classes and subclasses herein. In some embodiments, each R¹ is independently —N(R)C(O)R, wherein R is as defined in embodiments and classes and subclasses herein. In some embodiments, each R¹ is independently —N(R)C(O)NR₂, wherein R is as defined in embodiments and classes and subclasses herein. In some embodiments, each R¹ is independently —N(R)C(NR)NR₂, wherein R is as defined in embodiments and classes and subclasses herein. In some embodiments, each R¹ is independently —N(R)S(O)₂NR₂, wherein R is as defined in embodiments and classes and subclasses herein. In some embodiments, each R¹ is independently —N(R)S(O)₂R, wherein R is as defined in embodiments and classes and subclasses herein. In some embodiments, each R¹ is independently —P(O)R₂, wherein R is as defined in embodiments and classes and subclasses herein. In some embodiments, each R¹ is independently —P(O)(R)OR, wherein R is as defined in embodiments and classes and subclasses herein. In some embodiments, each R¹ is independently —B(OR)₂, wherein R is as defined in embodiments and classes and subclasses herein.

In some embodiments, each R¹ is independently R^{B} substituted by r¹ instances of R^{1C}. In some embodiments, R¹ (i.e., -L¹-R^{1A} taken together) is a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, each R¹ (i.e., -L¹-R^{1A} taken together) is independently halogen, —CN, —OR, or a C₁₋₆ aliphatic chain substituted with r¹ halogens. In some embodiments, each R¹ is independently halogen, —CN, —OR, or a C₁₋₆ aliphatic chain substituted with 0-5 halogens. In some embodiments, each R¹ is independently halogen, —CN, —O—(C₁₋₆ aliphatic chain substituted with 0-5 halogens), or a C₁₋₆ aliphatic chain substituted with 0-5 halogens. In some embodiments, each R¹ is independently halogen or a C₁₋₆ aliphatic chain substituted with 0-5 halogens. In some embodiments, each R¹ is independently halogen or a C₁₋₆ aliphatic chain substituted with 0-4 halogens. In some embodiments, each R¹ is independently halogen or a C₁₋₆ aliphatic chain substituted with 0-3 halogens. In some embodiments, each R¹ is independently halogen or a C₁₋₃ aliphatic chain substituted with 0-3 halogens. In some embodiments, each R¹ is independently halogen or a C₁₋₃ aliphatic chain substituted with 0-2 halogens.

In some embodiments, each R¹ is independently a halogen selected from Br, Cl, and F. In some embodiments, each R¹ is independently a halogen selected from Cl and F. In some embodiments, R¹ is Cl. In some embodiments, R¹ is F.

In some embodiments, at least one R¹ is halogen. In some embodiments, at least two R¹ are halogen. In some embodiments, at least three R¹ are halogen. In some embodiments, one instance of R¹ is Cl. In some embodiments, two instances of R¹ are Cl. In some embodiments, one instance of R¹ is F. In some embodiments, two instances of R¹ are F. In some embodiments, one instance of R¹ is Cl, and one instance of R¹ is F. In some embodiments, two instances of R¹ are Cl, and one instance of R¹ is F. In some embodiments, one instance of R¹ is Cl, and two instances of R¹ are F.

In some embodiments, each R¹ is independently a C₁₋₆ aliphatic chain substituted with 0-5 halogens. In some embodiments, each R¹ is independently a C₁₋₆ aliphatic chain substituted with 0-4 halogens. In some embodiments, each R¹ is independently a C₁₋₆ aliphatic chain substituted with 0-3 halogens. In some embodiments, each R¹ is independently a C₁₋₃ aliphatic chain substituted with 0-3 halogens. In some embodiments, each R¹ is independently a C₁₋₃ aliphatic chain substituted with 0-2 halogens.

In some embodiments, at least one R¹ is C₁₋₃ aliphatic optionally substituted with 1-3 halogen. In some embodiments, at least one R¹ is —O—C₁₋₃ aliphatic optionally substituted with 1-3 halogen.

In some embodiments, each R¹ (i.e., -L¹-R^{1A} taken together) is independently halogen, —OH, —OCH₃, or C₁₋₃ aliphatic optionally substituted with 1-3 halogen. In some embodiments, each R¹ is independently fluorine, chlorine, —OCH₃, or —CH₃. In some embodiments, R¹ is —OH. In some embodiments, R¹ is —CH₃. In some embodiments, R¹ is —OCH₃. In some embodiments, R¹ is —CF₃. In some embodiments, R¹ is —CHF₂.

In some embodiments, two instances of R¹ are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p² instances of R^{11C}; wherein p² and R^{11C} are as defined in embodiments and classes and subclasses herein.

In some embodiments, one instance of R^{T} and one instance of R¹ are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p⁴ instances of R^{T1C}; wherein p⁴ and R^{TLC} are as defined in embodiments and classes and subclasses herein.

In some embodiments, R¹ is selected from the groups depicted in the compounds in Table 1.

As defined generally above, each R² is independently -L²-R^{2A}; or two instances of R² are taken together with their intervening atoms to form a 3-7 membered saturated, partially unsaturated, or aromatic carbocyclic ring or a 3-7 membered saturated or partially unsaturated monocyclic heterocyclic ring having 1-3 heteroatoms independently selected from nitrogen, oxygen, and sulfur; wherein the ring is substituted with p³ instances of R^{22C}.

In some embodiments, each R² is independently -L²-R^{2A}. In some embodiments each R² is independently R^{2A}.

In some embodiments, R² (i.e., -L²-R^{2A} taken together) is —N(R)—R^{2A}, wherein R and R^{2A} are as defined in embodiments and classes and subclasses herein. In some embodiments, R² (i.e., -L²-R^{2A} taken together) is —NH—R^{2A}, wherein R^{2A} is as defined in embodiments and classes and subclasses herein. In some embodiments, R² (i.e., -L²-R^{2A} taken together) is —CH(R^{L})—R^{2A}, wherein R and R^{2A} are as defined in embodiments and classes and subclasses herein. In some embodiments, R² (i.e., -L²-R^{2A} taken together) is —CH₂—R^{2A}, wherein R^{2A} is as defined in embodiments and classes and subclasses herein. In some embodiments, R² (i.e., -L²-R^{2A} taken together) is —N(R)C(O)—R^{2A}, wherein R and R^{2A} are as defined in embodiments and classes and subclasses herein. In some embodiments, R² (i.e., -L²-R^{2A} taken together) is —NHC(O)—R^{2A}, wherein R^{2A} is as defined in embodiments and classes and subclasses herein. In some embodiments, R² (i.e., -L²-R^{2A} taken together) is —N(R)C(O)CH(R^{L})—R^{2A} wherein R and R^{2A} are as defined in embodiments and classes and subclasses herein. In some embodiments, R² (i.e., -L²-R^{2A} taken together) is —NHC(O)CH₂—R^{2A}, wherein R² is as defined in embodiments and classes and subclasses herein. In some embodiments, R² (i.e., -L²-R^{2A} taken together) is —N(R)C(O)N(R)—R^{2A}, wherein R and R^{2A} are as defined in embodiments and classes and subclasses herein. In some embodiments, R² (i.e., -L²-R^{2A} taken together) is —NHC(O)NH—R^{2A}, wherein R^{2A} is as defined in embodiments and classes and subclasses herein. In some embodiments, R² (i.e., -L²-R^{2A} taken together) is —N(R)C(O)CH(R^{L})O—R^{2A}, wherein R and R^{2A} are as defined in embodiments and classes and subclasses herein. In some embodiments, R² (i.e., -L²-R^{2A} taken together) is —NHC(O)CH₂O—R^{2A}, wherein R^{2A} is as defined in embodiments and classes and subclasses herein. In some embodiments, R² (i.e., -L²-R^{2A} taken together) is —CH(R^{L})N(R)—R^{2A}, wherein R and R^{2A} are as defined in embodiments and classes and subclasses herein. In some embodiments, R² (i.e., -L²-R^{2A} taken together) is —CH(R^{L})O—R^{2A}, wherein R and R^{2A} are as defined in embodiments and classes and subclasses herein.

In some embodiments, R² is —N(R)—R^{2A}, —N(R)C(O)—R^{2A}, —CH(R^{L})N(R)—R^{2A}, —N(R)C(O)CH(R^{L})—R^{2A}, —CH(R^{L})O—R^{2A}, —CH(R^{L})—R^{2A}, or —R^{2A}. In some embodiments, R² is —N(R)—R^{2A}, —N(R)C(O)—R^{2A}, —CH(R^{L})N(R)—R^{2A}, or —R^{2A}. In some embodiments, R² is —N(R)—R^{2A}, —N(R)C(O)—R^{2A}, or —R^{2A}. In some embodiments, R² is —N(R)C(O)—R^{2A} or —R^{2A}.

In some embodiments, R² is —N(H)—R^{2A}, —N(H)C(O)—R^{2A}, —CH₂N(H)—R^{2A}, —N(H)C(O)CH₂—R^{2A}, —CH₂O—R^{2A}, —CH₂—R^{2A}, or —R^{2A}. In some embodiments, R² is —N(H)—R^{2A}, —N(H)C(O)—R^{2A}, —CH₂N(H)—R^{2A}, or —R^{2A}. In some embodiments, R² is —N(H)—R^{2A}, —N(H)C(O)—R^{2A}, or —R^{2A}. In some embodiments, R² is —N(H)C(O)—R^{2A} or —R^{2A}.

In some embodiments, R² (i.e., -L²-R^{2A} taken together) is

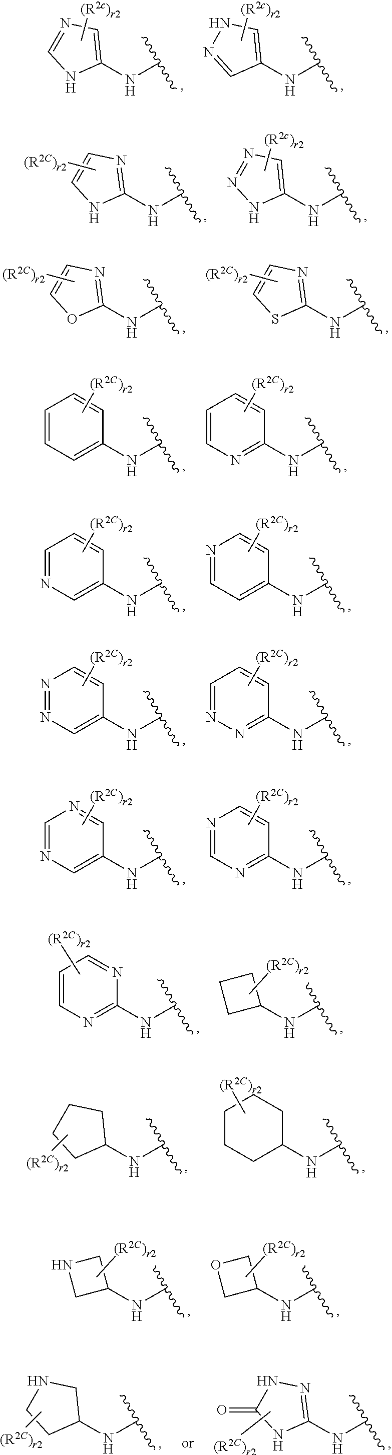

wherein R^{2C} and r² are as defined in the embodiments and classes and subclasses herein.

In some embodiments, R² (i.e., -L²-R^{2A} taken together) is

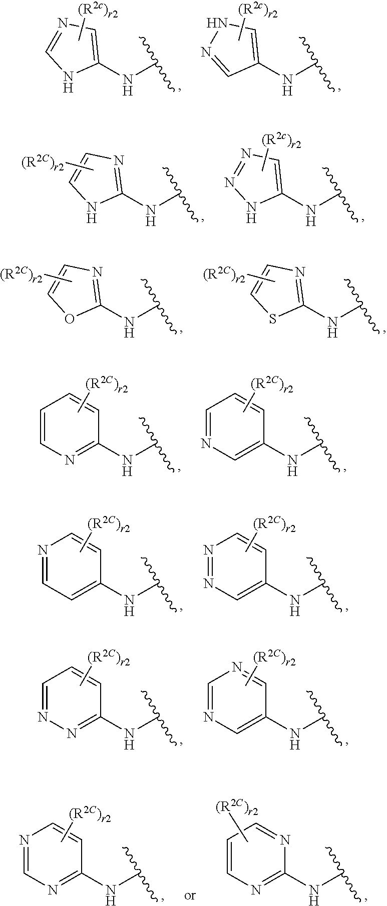

wherein R^{2C} and r² are as defined in the embodiments and classes and subclasses herein.

In some embodiments, R² (i.e., -L²-R^{2A} taken together) is

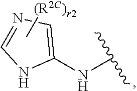

wherein R^{2C} and r² are as defined in the embodiments and classes and subclasses herein. In some embodiments, R² (i.e., -L²-R^{2A} taken together) is

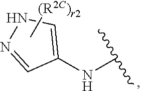

wherein R^{2C} and r² are as defined in the embodiments and classes and subclasses herein. In some embodiments, R² (i.e., -L²-R^{2A} taken together) is

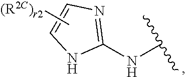

wherein R^{2C} and r² are as defined in the embodiments and classes and subclasses herein. In some embodiments, R² (i.e., -L²-R^{2A} taken together) is

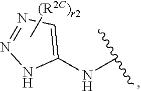

wherein R^{2C} and r² are as defined in the embodiments and classes and subclasses herein. In some embodiments, R² (i.e., -L²-R^{2A} taken together) is

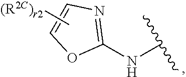

wherein R^{2C} and r² are as defined in the embodiments and classes and subclasses herein. In some embodiments, R² (i.e., -L²-R^{2A} taken together) is

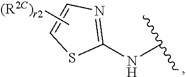

wherein R^{2C} and r² are as defined in the embodiments and classes and subclasses herein. In some embodiments, R² (i.e., -L²-R^{2A} taken together) is

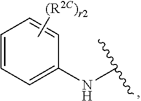

wherein R^{2C} and r² are as defined in the embodiments and classes and subclasses herein. In some embodiments, R² (i.e., -L²-R^{2A} taken together) is

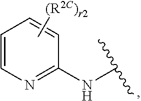

wherein R^{2C} and r² are as defined in the embodiments and classes and subclasses herein. In some embodiments, R² (i.e., -L²-R^{2A} taken together) is

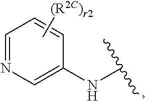

wherein R^{2C} and r² are as defined in the embodiments and classes and subclasses herein. In some embodiments, R² (i.e., -L²-R^{2A} taken together) is

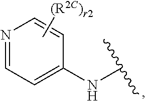

wherein R^{2C} and r² are as defined in the embodiments and classes and subclasses herein. In some embodiments, R² (i.e., -L²-R^{2A} taken together) is

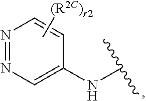

wherein R^{2C} and r² are as defined in the embodiments and classes and subclasses herein. In some embodiments, R² (i.e., -L²-R^{2A} taken together) is

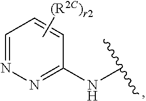

wherein R^{2C} and r² are as defined in the embodiments and classes and subclasses herein. In some embodiments, R² (i.e., -L²-R^{2A} taken together) is

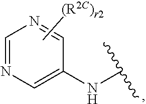

wherein R^{2C} and r² are as defined in the embodiments and classes and subclasses herein. In some embodiments, R² (i.e., -L²-R^{2A} taken together) is

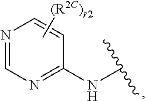

wherein R^{2C} and r² are as defined in the embodiments and classes and subclasses herein. In some embodiments, R² (i.e., -L²-R^{2A} taken together) is

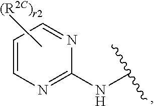

wherein R^{2C} and r² are as defined in the embodiments and classes and subclasses herein.

In some embodiments, R² (i.e., -L²-R^{2A} taken together) is

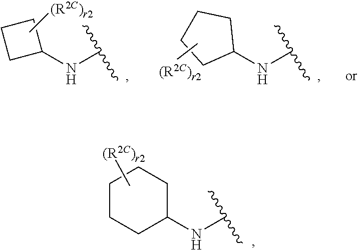

wherein R^{2C} and r² are as defined in the embodiments and classes and subclasses herein. In some embodiments, R² (i.e., -L²-R^{2A} taken together) is

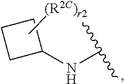

wherein R^{2C} and r² are as defined in the embodiments and classes and subclasses herein. In some embodiments, R² (i.e., -L²-R^{2A} taken together) is

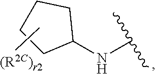

wherein R^{2C} and r² are as defined in the embodiments and classes and subclasses herein. In some embodiments, R² (i.e., -L²-R^{2A} taken together) is

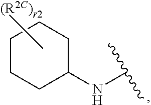

wherein R^{2C} and r² are as defined in the embodiments and classes and subclasses herein.

In some embodiments, R² (i.e., -L²-R^{2A} taken together) is

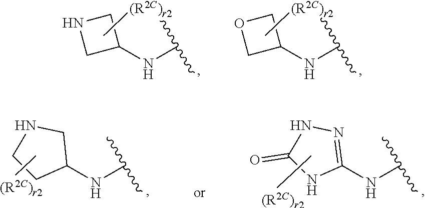

wherein R^{2C} and r² are as defined in the embodiments and classes and subclasses herein. In some embodiments, R² (i.e., -L²-R^{2A} taken together) is

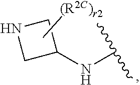

wherein R^{2C} and r² are as defined in the embodiments and classes and subclasses herein. In some embodiments, R² (i.e., -L²-R^{2A} taken together) is

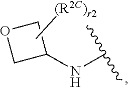

wherein R^{2C} and r² are as defined in the embodiments and classes and subclasses herein. In some embodiments, R² (i.e., -L²-R^{2A} taken together) is

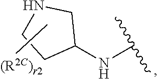

wherein R^{2C} and r² are as defined in the embodiments and classes and subclasses herein. In some embodiments, R² (i.e., -L²-R^{2A} taken together) is

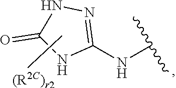

wherein R^{2C} and r² are as defined in the embodiments and classes and subclasses herein.

In some embodiments, R² (i.e., -L²-R^{2A} taken together) is

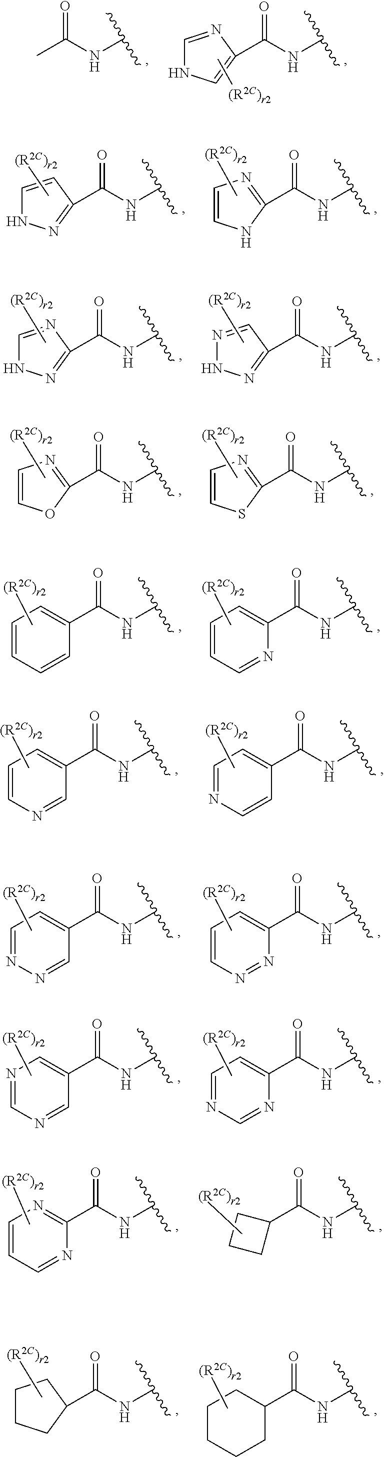

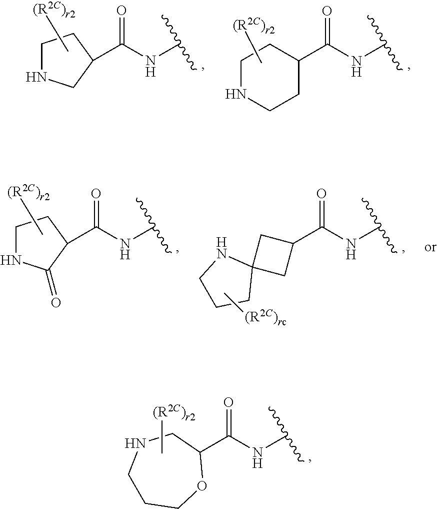

wherein R^{2C} and r² are defined in the embodiments and classes and subclasses herein.

In some embodiments, R² (i.e., -L²-R^{2A} taken together) is

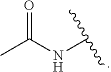 [SMILES: *NC(C)=O]

In some embodiments, R² (i.e., -L²-R^{2A} taken together) is

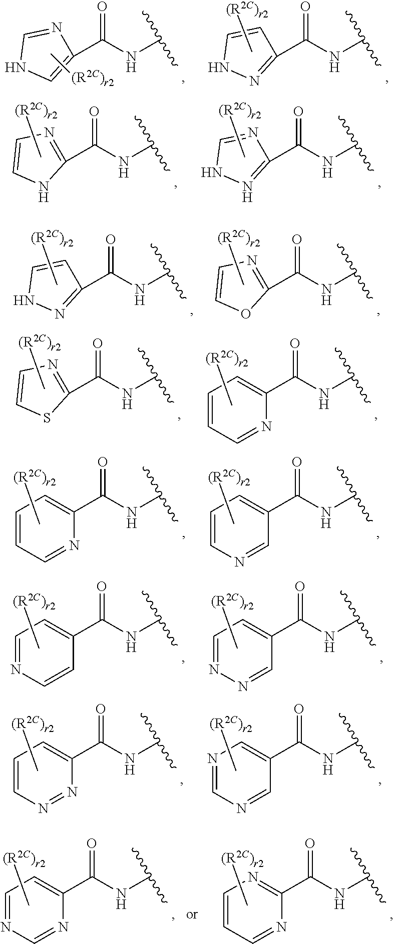

wherein R^{2C} and r² are as defined in the embodiments and classes and subclasses herein.

In some embodiments, R² (i.e., -L²-R^{2A} taken together) is

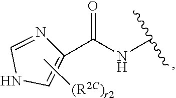

wherein R^{2C} and r² are as defined in the embodiments and classes and subclasses herein. In some embodiments, R² (i.e., -L²-R^{2A} taken together) is

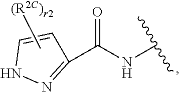

wherein R^{2C} and r² are as defined in the embodiments and classes and subclasses herein. In some embodiments, R² (i.e., -L²-R^{2A} taken together) is

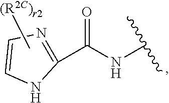

wherein R^{2C} and r² are as defined in the embodiments and classes and subclasses herein. In some embodiments, R² (i.e., -L²-R^{2A} taken together) is

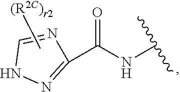

wherein R^{2C} and r² are as defined in the embodiments and classes and subclasses herein. In some embodiments, R² (i.e., -L²-R^{2A} taken together) is

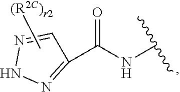

wherein R^{2C} and r² are as defined in the embodiments and classes and subclasses herein. In some embodiments, R² (i.e., -L²-R^{2A} taken together) is

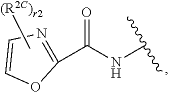

wherein R^{2C} and r² are as defined in the embodiments and classes and subclasses herein. In some embodiments, R² (i.e., -L²-R^{2A} taken together) is

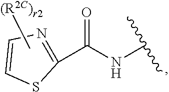

wherein R^{2C} and r² are as defined in the embodiments and classes and subclasses herein. In some embodiments, R² (i.e., -L²-R^{2A} taken together) is

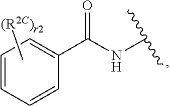

wherein R^{2C} and r² are as defined in the embodiments and classes and subclasses herein. In some embodiments, R² (i.e., -L²-R^{2A} taken together) is

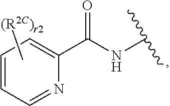

wherein R^{2C} and r² are as defined in the embodiments and classes and subclasses herein. In some embodiments, R² (i.e., -L²-R² taken together) is

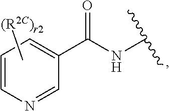

wherein R^{2C} and r² are as defined in the embodiments and classes and subclasses herein. In some embodiments, R² (i.e., -L²-R^{2A} taken together) is

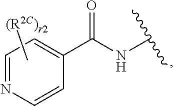

wherein R^{2C} and r² are as defined in the embodiments and classes and subclasses herein. In some embodiments, R² (i.e., -L²-R^{2A} taken together) is

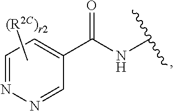

wherein R^{2C} and r² are as defined in the embodiments and classes and subclasses herein. In some embodiments, R² (i.e., -L²-R^{2A} taken together) is

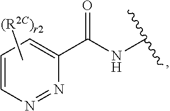

wherein R^{2C} and r² are as defined in the embodiments and classes and subclasses herein. In some embodiments, R² (i.e., -L²-R^{2A} taken together) is

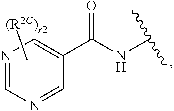

wherein R^{2C} and r² are as defined in the embodiments and classes and subclasses herein. In some embodiments, R² (i.e., -L²-R^{2A} taken together) is

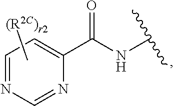

wherein R^{2C} and r² are as defined in the embodiments and classes and subclasses herein. In some embodiments, R² (i.e., -L²-R^{2A} taken together) is

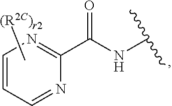

wherein R^{2C} and r² are as defined in the embodiments and classes and subclasses herein.

In some embodiments, R² (i.e., -L²-R^{2A} taken together) is

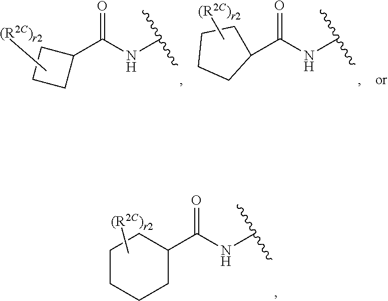

wherein R^{2C} and r² are as defined in the embodiments and classes and subclasses herein. In some embodiments, R² (i.e., -L²-R^{2A} taken together) is

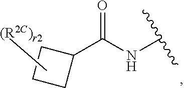

wherein R^{2C} and r² are as defined in the embodiments and classes and subclasses herein. In some embodiments, R² (i.e., -L²-R^{2A} taken together) is

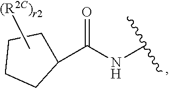

wherein R^{2C} and r² are as defined in the embodiments and classes and subclasses herein. In some embodiments, R² (i.e., -L²-R^{2A} taken together) is

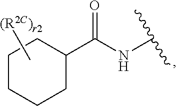

wherein R^{2C} and r² are as defined in the embodiments and classes and subclasses herein.

In some embodiments, R² (i.e., -L²-R^{2A} taken together) is

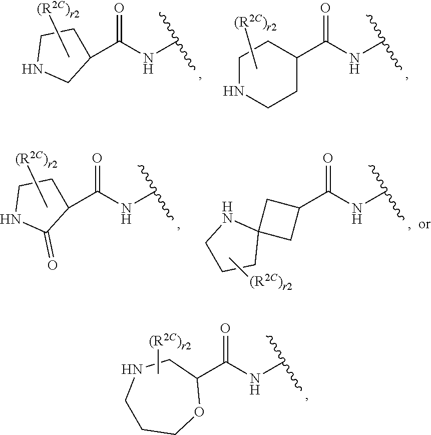

wherein R^{2C} and r² are as defined in the embodiments and classes and subclasses herein. In some embodiments, R² (i.e., -L²-R^{2A} taken together) is

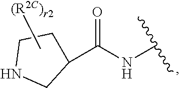

wherein R^{2C} and r² are as defined in the embodiments and classes and subclasses herein. In some embodiments, R² (i.e., -L²-R^{2A} taken together) is

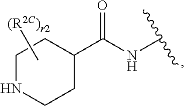

wherein R^{2C} and r² are as defined in the embodiments and classes and subclasses herein. In some embodiments, R² (i.e., -L²-R^{2A} taken together) is

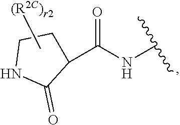

wherein R^{2C} and r² are as defined in the embodiments and classes and subclasses herein. In some embodiments, R² (i.e., L²-R^{2A} taken together) is

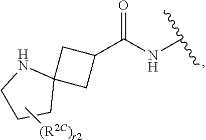

wherein R^{2C} and r² are as defined in the embodiments and classes and subclasses herein. In some embodiments, R² (i.e., -L²-R^{2A} taken together) is

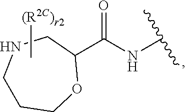

wherein R^{2C} and r² are as defined in the embodiments and classes and subclasses herein.

In some embodiments, R² (i.e., -L²-R^{2A} taken together) is

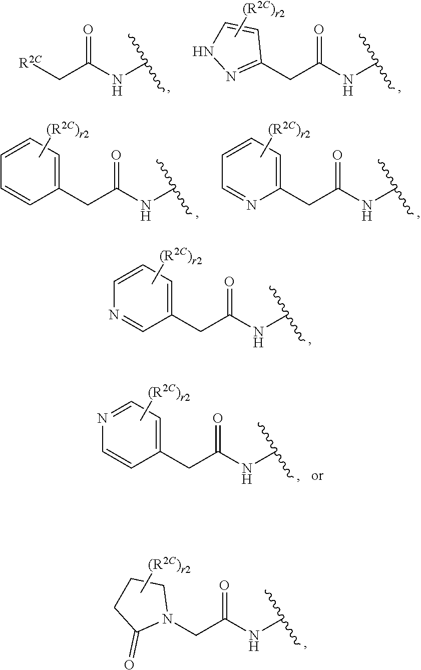

wherein R^{2C} and r² are as defined in the embodiments and classes and subclasses herein.

In some embodiments, R² (i.e., -L²-R^{2A} taken together) is

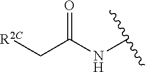

wherein R^{2C} is as defined in the embodiments and classes and subclasses herein. In some embodiments, R² (i.e., -L²-R^{2A} taken together) is

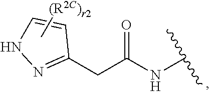

wherein R^{2C} and r² are as defined in the embodiments and classes and subclasses herein. In some embodiments, R² (i.e., -L²-R^{2A} taken together) is

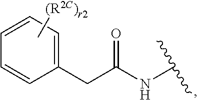

wherein R^{2C} and r² are as defined in the embodiments and classes and subclasses herein. In some embodiments, R² (i.e., -L²-R^{2A} taken together) is

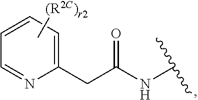

wherein R^{2C} and r² are as defined in the embodiments and classes and subclasses herein. In some embodiments, R² (i.e., -L²-R^{2A} taken together) is

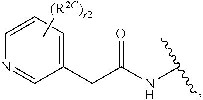

wherein R^{2C} and r² are as defined in the embodiments and classes and subclasses herein. In some embodiments, R² (i.e., -L²-R^{2A} taken together) is

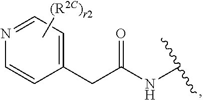

H wherein R^{2C} and r² are as defined in the embodiments and classes and subclasses herein. In some embodiments, R² (i.e., -L²-R^{2A} taken together) is

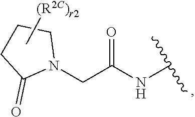

wherein R^{2C} and r² are as defined in the embodiments and classes and subclasses herein.

In some embodiments, R² (i.e., -L²-R^{2A} taken together) is

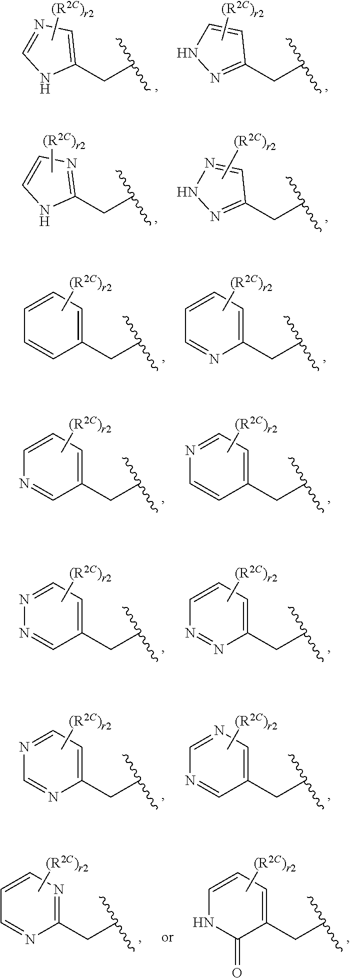

wherein R^{2A}, R^{2C}, and r² are as defined in the embodiments and classes and subclasses herein.

In some embodiments, R² (i.e., -L²-R^{2A} taken together) is

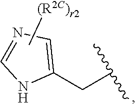

wherein R^{2C} and r² are as defined in embodiments and classes and subclasses herein. In some embodiments, R² (i.e., -L²-R^{2A} taken together) is

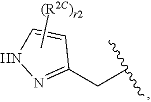

wherein R^{2C} and r² are as defined in embodiments and classes and subclasses herein. In some embodiments, R² (i.e., -L²-R^{2A} taken together) is

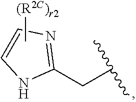

wherein R^{2C} and r² are as defined in embodiments and classes and subclasses herein. In some embodiments, R² (i.e., -L²-R^{2A} taken together) is

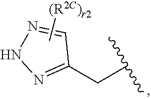

wherein R^{2C} and r² are as defined in embodiments and classes and subclasses herein. In some embodiments, R² (i.e., -L²-R^{2A} taken together) is

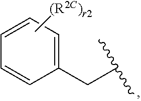

wherein R^{2C} and r² are as defined in embodiments and classes and subclasses herein. In some embodiments, R² (i.e., -L²-R^{2A} taken together) is

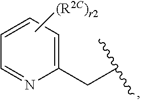

wherein R^{2C} and r² are as defined in embodiments and classes and subclasses herein. In some embodiments, R² (i.e., -L²-R^{2A} taken together) is

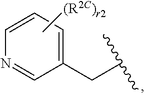

wherein R^{2C} and r² are as defined in embodiments and classes and subclasses herein. In some embodiments, R² (i.e., -L²-R^{2A} taken together) is

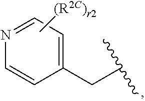

wherein R^{2C} and r² are as defined in embodiments and classes and subclasses herein. In some embodiments, R² (i.e., -L²-R^{2A} taken together) is

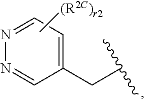

wherein R^{2C} and r² are as defined in embodiments and classes and subclasses herein. In some embodiments, R² (i.e., -L²-R^{2A} taken together) is

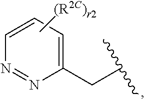

wherein R^{2C} and r² are as defined in embodiments and classes and subclasses herein. In some embodiments, R² (i.e., -L²-R^{2A} taken together) is

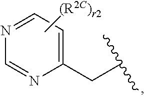

wherein R^{2C} and r² are as defined in embodiments and classes and subclasses herein. In some embodiments, R² (i.e., -L²-R^{2A} taken together) is

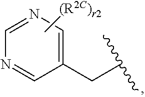

wherein R^{2C} and r² are as defined in embodiments and classes and subclasses herein. In some embodiments, R² (i.e., -L²-R^{2A} taken together) is

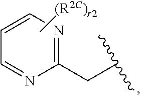

wherein R^{2C} and r² are as defined in embodiments and classes and subclasses herein. In some embodiments, R² (i.e., -L²-R^{2A} taken together) is

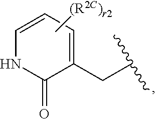

wherein R^{2C} and r² are as defined in embodiments and classes and subclasses herein.

In some embodiments, each R² (i.e., -L²-R^{2A} taken together) is independently halogen, —OH, —OCH₃, or C₁₋₃ aliphatic optionally substituted with 1-3 halogen. In some embodiments, each R² is independently fluorine, chlorine, —OCH₃, or —CH₃. In some embodiments, R² is —OH. In some embodiments, R² is —CH₃. In some embodiments, R² is —OCH₃. In some embodiments, R² is —CF₃. In some embodiments, R² is —CHF₂.

In some embodiments, two instances of R² are taken together with their intervening atoms to form a 3-7 membered saturated, partially unsaturated, or aromatic carbocyclic ring or a 3-7 membered saturated or partially unsaturated monocyclic heterocyclic ring having 1-3 heteroatoms independently selected from nitrogen, oxygen, and sulfur; wherein the ring is substituted with p³ instances of R^{22C}. In some embodiments, two instances of R² are taken together with their intervening atoms to form a 3-7 membered saturated, partially unsaturated, or aromatic carbocyclic ring; wherein the ring is substituted with p3 instances of R^{22C}. In some embodiments, two instances of R² are taken together with their intervening atoms to form a 6-membered aromatic carbocyclic ring; wherein the ring is substituted with p³ instances of R^{22C}.

In some embodiments, R² is selected from the groups depicted in the compounds in Table 1.

As defined generally above, each R^{T} is independently -L^{T}-R^{TA}; or two instances of R^{T} are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p¹ instances of R^{TTC}; or one instance of R^{T} and one instance of R¹ are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p⁴ instances of R^{T1C}; or one instance of R^{T} and one instance of R^{L} are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p⁵ instances of R^{TLC}.

In some embodiments, each R^{T} is independently -L^{T}-R^{TA}. In some embodiments, each R^{T} is independently —R^{TA}. In some embodiments, each R^{T} is independently R^{A}. In some embodiments, each R^{T} is independently R^{B} substituted by r³ instances of R^{TC}.

In some embodiments, R^{T} (i.e., -L^{T}-R^{TA} taken together) is a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted with r³ instances of R^{TC}.

In some embodiments, R^{T} is a C₁₋₆ aliphatic chain; adamantyl; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted with r³ instances of R^{TC}.

In some embodiments, R^{T} is a 3-7 membered saturated or partially unsaturated monocyclic carbocyclic ring or a 5-12 membered saturated or partially unsaturated bicyclic carbocyclic ring; each of which is substituted by r³ instances of R^{TC}. In some embodiments, R^{T} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted with r³ instances of R^{TC}.

In some embodiments, R^{T} is a 3-7 membered saturated monocyclic carbocyclic ring; a 5-12 membered saturated bicyclic carbocyclic ring; a 3-7 membered saturated monocyclic heterocyclic ring having 1-2 heteroatoms independently selected from nitrogen, oxygen, and sulfur; or a 7-12 membered saturated bicyclic heterocyclic ring having 1-4 heteroatoms independently selected from nitrogen, oxygen, and sulfur; each of which is substituted with r³ instances of R^{TC}. In some embodiments, R^{T} is a 3-7 membered saturated monocyclic carbocyclic ring; or a 5-12 membered saturated bicyclic carbocyclic ring; each of which is substituted with r³ instances of R^{TC}. In some embodiments, R^{T} is a 3-7 membered saturated monocyclic heterocyclic ring having 1-2 heteroatoms independently selected from nitrogen, oxygen, and sulfur; or a 7-12 membered saturated bicyclic heterocyclic ring having 1-4 heteroatoms independently selected from nitrogen, oxygen, and sulfur; each of which is substituted with r³ instances of R^{TC}.

In some embodiments, Rr (i.e., -L^{T}-R^{TA} taken together) is a C₁₋₆ aliphatic chain substituted with r³ instances of R^{TC}. In some embodiments, R^{T} (i.e., -L^{T}-R^{TA} taken together) is phenyl substituted with r³ instances of R^{TC}. In some embodiments, R^{T} (i.e., -L^{T}-R^{TA} taken together) is naphthyl substituted with r³ instances of R^{TC}. In some embodiments, R^{T} (i.e., -L^{T}-R^{TA} taken together) is cubanyl substituted with r³ instances of R^{TC}. In some embodiments, R^{T} (i.e., -L^{T}-R^{TA} taken together) is adamantyl substituted with r³ instances of R^{TC}. In some embodiments, R^{T} (i.e., -L^{T}-R^{TA} taken together) is a 5-6 membered monocyclic heteroaryl ring having 1-4 heteroatoms independently selected from nitrogen, oxygen, and sulfur, wherein the ring is substituted with r³ instances of R^{TC}. In some embodiments, R^{T} (i.e., -L^{T}-R^{TA} taken together) is an 8-10 membered bicyclic heteroaryl ring having 1-4 heteroatoms independently selected from nitrogen, oxygen, and sulfur, wherein the ring is substituted with r³ instances of R^{TC}. In some embodiments, R^{T} (i.e., -L^{T}-R^{TA} taken together) is a 3-7 membered saturated or partially unsaturated monocyclic carbocyclic ring, wherein the ring is substituted with r³ instances of R^{TC}. In some embodiments, R¹ (i.e., -L^{T}-R^{A} taken together) is a 5-12 membered saturated or partially unsaturated bicyclic carbocyclic ring, wherein the ring is substituted with r³ instances of R^{TC}. In some embodiments, R^{T}(i.e., -L^{T}-R^{TA} taken together) is a 3-7 membered saturated or partially unsaturated monocyclic heterocyclic ring having 1-2 heteroatoms independently selected from nitrogen, oxygen, and sulfur, wherein the ring is substituted with r³ instances of R^{TC}. In some embodiments, R^{T} (i.e., -L^{T}-R^{TA} taken together) is a 7-12 membered saturated or partially unsaturated bicyclic heterocyclic ring having 1-4 heteroatoms independently selected from nitrogen, oxygen, and sulfur, wherein the ring is substituted with r³ instances of R^{TC}.

In some embodiments, R^{T} is

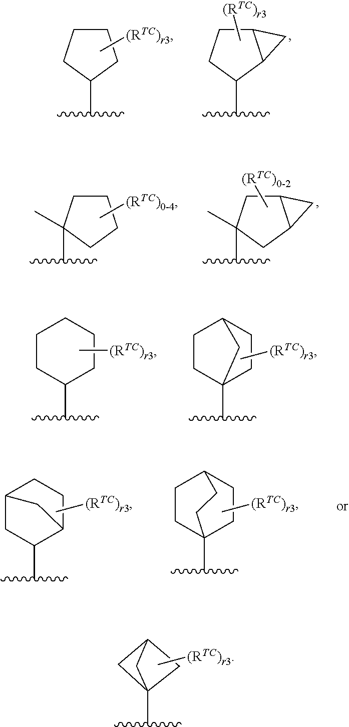

In some embodiments, R^{T} is

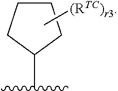

In some embodiments, R^{T} is

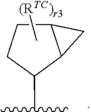

In some embodiments, R^{T} is

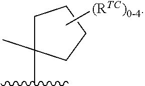

In some embodiments, R^{T} is

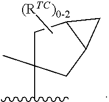

In some embodiments, R^{T} is

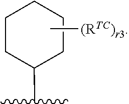

In some embodiments, R^{T} is

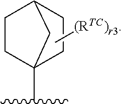

In some embodiments, R^{T} is

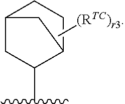

In some embodiments, R^{T} is

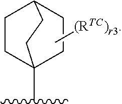

In some embodiments, R^{T} is

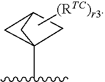

In some embodiments, R^{T} is CF3. In some embodiments, R^{T} is C₁₋₆ alkyl substituted by r³ instances of R^{TC}. In some embodiments, R^{T} is C₃₋₈ cycloalkyl substituted by r³ instances of R^{TC}.

In some embodiments, two instances of R^{T} are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p¹ instances of R^{TTC}.

In some embodiments, one instance of R^{T} and one instance of R¹ are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p⁴ instances of R^{T1C}.

In some embodiments, one instance of R^{T} and one instance of R^{L} are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p⁵ instances of R^{TLC}.

In some embodiments, R^{T} is selected from the groups depicted in the compounds in Table 1.

As defined generally above, L¹ is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L¹ is a covalent bond. In some embodiments, L¹ is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L¹ is a C₁₋₄ bivalent saturated or unsaturated, straight or branched hydrocarbon chain.

In some embodiments, L¹ is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L¹ is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L¹ is a C₁₋₂ bivalent saturated or unsaturated hydrocarbon chain.

In some embodiments, L¹ is selected from the groups depicted in the compounds in Table 1.

As defined generally above, L² is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L² is a covalent bond. In some embodiments, L² is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L² is a C₁₋₄ bivalent saturated or unsaturated, straight or branched hydrocarbon chain.

In some embodiments, L² is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L² is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L² is a C₁₋₂ bivalent saturated or unsaturated hydrocarbon chain.

In some embodiments, L² is —N(R)—, —N(R)C(O)—, —CH(R^{L})N(R)—, —N(R)C(O)CH(R^{L})—, —CH(R^{L})O—, —CH(R^{L})—, or a covalent bond. In some embodiments, R² is —N(R)—, —N(R)C(O)—, —CH(R^{L})N(R)—, or a covalent bond. In some embodiments, R² is —N(R)—, —N(R)C(O)—, or a covalent bond. In some embodiments, R² is —N(R)C(O)— or a covalent bond.

In some embodiments, R² is —N(H)—, —N(H)C(O)—, —CH₂N(H)—, —N(H)C(O)CH₂—, —CH₂O—, —CH₂—, or a covalent bond. In some embodiments, R² is —N(H)—, —N(H)C(O)—, —CH₂N(H)—, or a covalent bond. In some embodiments, R² is —N(H)—, —N(H)C(O)—, or a covalent bond. In some embodiments, R² is —N(H)C(O)— or a covalent bond.

In some embodiments, L² is —N(R)C(O)— or —N(R)C(O)N(R)—. In some embodiments, L² is —N(H)C(O)— or —N(H)C(O)N(H)—. In some embodiments, L² is —N(R)C(O)—. In some embodiments, L² is —N(H)C(O)—. In some embodiments, L² is —N(R)C(O)N(R)—. In some embodiments, L² is —N(H)C(O)N(H)—. In some embodiments, L² is —N(R)—. In some embodiments, L² is —N(H)—. In some embodiments, L² is a covalent bond. In some embodiments, L² is —CH(R^{L})N(R)—. In some embodiments, L² is —N(R)C(O)CH(R^{L})—. In some embodiments, L² is —CH(R^{L})O—. In some embodiments, L² is —CH(R^{L})—.

In some embodiments, L² is selected from the groups depicted in the compounds in Table 1.

As defined generally above, L^{Q} is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Q} is a covalent bond. In some embodiments, L^{Q} is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Q} is a C₁₋₄ bivalent saturated or unsaturated, straight or branched hydrocarbon chain.

In some embodiments, L^{Q} is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Q} is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L^{Q} is a C₁₋₂ bivalent saturated or unsaturated hydrocarbon chain.

In some embodiments, L^{Q} is —C(O)N(R)—, —C(O)N(R)CH₂—, —N(R)—, —CH₂C(O)N(R)—, —N(R)C(O)N(R)—, or a covalent bond. In some embodiments, L^{Q} is —C(O)N(H)—, —C(O)N(H)CH₂—, —N(H)—, —CH₂C(O)N(H)—, —N(H)C(O)N(H)—, or a covalent bond. In some embodiments, L^{Q} is —C(O)N(H)—, —C(O)N(H)CH₂—, or a covalent bond. In some embodiments, L^{Q} is —C(O)N(H)— or —C(O)N(H)CH₂—. In some embodiments, L^{Q} is —C(O)N(H)—. In some embodiments, L^{Q} is —C(O)N(H)CH₂—. In some embodiments, L^{Q} is —N(H)—. In some embodiments, L^{Q} is —CH₂C(O)N(H)—. In some embodiments, L^{Q} is —N(H)C(O)N(H)—. In some embodiments, L^{Q} is a covalent bond.

In some embodiments, L^{Q} is selected from the groups depicted in the compounds in Table 1.

As defined generally above, L^{T} is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, LT is a covalent bond. In some embodiments, L^{T} is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{X} is a C₁₋₄ bivalent saturated or unsaturated, straight, or branched hydrocarbon chain.

In some embodiments, L^{T} is a C₁₋₂ bivalent saturated or unsaturated hydrocarbon chain wherein one or two methylene units of the chain are optionally and independently replaced by —CH(R^{L}), —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{T} is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L^{T} is a C₁₋₂ bivalent saturated or unsaturated hydrocarbon chain.

In some embodiments, L^{T} is selected from the groups depicted in the compounds in Table 1.

As defined generally above, each R^{1A} is independently R^{A} or R^{B} substituted by r¹ instances of R^{1C}. In some embodiments, each R^{1A} is independently R^{A}. In some embodiments, each R^{1A} is independently R^{B} substituted by r¹ instances of R^{1C}.

In some embodiments, R^{1A} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{1A} is substituted by r¹ instances of R^{1C}.

In some embodiments, R^{1A} is phenyl substituted by r¹ instances of R^{1C}. In some embodiments, R^{1A} is an 8-10 membered bicyclic heteroaryl ring having 1-4 heteroatoms independently selected from nitrogen, oxygen, and sulfur, wherein R^{1A} is substituted by r¹ instances of R^{1C}. In some embodiments, R^{1A} is phenyl or an 8-10 membered bicyclic heteroaryl ring having 1-4 heteroatoms independently selected from nitrogen, oxygen, and sulfur, wherein R^{1A} is substituted by r¹ instances of R^{1C}.

In some embodiments, R^{1A} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; or a 3-7 membered saturated or partially unsaturated monocyclic carbocyclic ring; wherein R^{1A} is substituted by r¹ instances of R^{1C}.

In some embodiments, R^{1A} is phenyl substituted by r¹ instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{1A} is an 8-10 membered bicyclic heteroaryl ring having 1-4 heteroatoms independently selected from nitrogen, oxygen, and sulfur, wherein R^{1A} is substituted by r¹ instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{1A} is phenyl or an 8-10 membered bicyclic heteroaryl ring having 1-4 heteroatoms independently selected from nitrogen, oxygen, and sulfur, wherein R^{1A} is substituted by r¹ instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{1A} is phenyl substituted by 1-3 instances of R^{1C}. In some embodiments, R^{1A} is phenyl substituted by 2 instances of R^{1C}. In some embodiments, R^{1A} is phenyl substituted by 1 instance of R^{1C}.

In some embodiments, R^{1A} is phenyl substituted by 1-3 instances of a group independently selected from halogen, —CN, —O-(optionally substituted C₁₋₆ aliphatic), and an optionally substituted C₁₋₆ aliphatic. In some embodiments, R^{1A} is phenyl substituted by 1-3 instances of a group independently selected from halogen and C₁₋₃ aliphatic optionally substituted with 1-3 halogen. In some embodiments, R^{1A} is phenyl substituted by 1-3 instances of a group independently selected from fluorine, chlorine, —CH₃, —CHF₂, and —CF₃.

In some embodiments, R^{1A} is phenyl substituted by 2 instances of a group independently selected from halogen, —CN, —O-(optionally substituted C₁₋₆ aliphatic), and an optionally substituted C₁₋₆ aliphatic. In some embodiments, R^{1A} is phenyl substituted by 2 instances of a group independently selected from halogen and C₁₋₃ aliphatic optionally substituted with 1-3 halogen. In some embodiments, R^{1A} is phenyl substituted by 2 instances of a group independently selected from fluorine, chlorine, —CH₃, —CHF₂, and —CF₃.

In some embodiments, R^{1A} is phenyl substituted by one group selected from halogen, —CN, —O-(optionally substituted C₁₋₆ aliphatic), and an optionally substituted C₁₋₆ aliphatic. In some embodiments, R^{1A} is phenyl substituted by one halogen or C₁₋₃ aliphatic group optionally substituted with 1-3 halogen. In some embodiments, R^{1A} is phenyl substituted by one fluorine, chlorine, —CH₃, —CHF₂, or —CF₃.

In some embodiments, each R^{1A} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, each R^{1A} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{1A} is oxo. In some embodiments, each R^{1A} is independently halogen. In some embodiments, R^{1A} is —CN. In some embodiments, R^{1A} is —NO₂. In some embodiments, each R^{1A} is independently —OR. In some embodiments, each R^{1A} independently —SR. In some embodiments, each R^{1A} is independently —NR₂. In some embodiments, each R^{1A} is independently —S(O)₂R. In some embodiments, each R^{1A} is independently —S(O)₂NR₂. In some embodiments, R^{1A} is —S(O)₂F. In some embodiments, each R^{1A} is independently —S(O)R. In some embodiments, each R^{1A} is independently —S(O)NR₂. In some embodiments, each R^{1A} is independently —S(O)(NR)R. In some embodiments, each R^{1A} is independently —C(O)R. In some embodiments, each R^{1A} is independently —C(O)OR. In some embodiments, each R^{1A} is independently —C(O)NR₂. In some embodiments, each R^{1A} is independently —C(O)N(R)OR. In some embodiments, each R^{1A} is independently —OC(O)R. In some embodiments, each R^{1A} is independently —OC(O)NR₂. In some embodiments, each R^{1A} is independently —N(R)C(O)OR. In some embodiments, each R^{1A} is independently —N(R)C(O)R. In some embodiments, each R^{1A} is independently —N(R)C(O)NR₂. In some embodiments, each R¹ is independently —N(R)C(NR)NR₂. In some embodiments, each R^{1A} is independently —N(R)S(O)₂NR₂. In some embodiments, each R^{1A} is independently —N(R)S(O)₂R. In some embodiments, each R^{1A} is independently —P(O)R₂. In some embodiments, each R^{1A} is independently —P(O)(R)OR. In some embodiments, each R^{1A} is independently —B(OR)₂. In some embodiments, R^{1A} is deuterium.

In some embodiments, R^{1A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{1A} is halogen, —CN, or —NO₂. In some embodiments, R^{1A} is —OR, —SR, or —NR₂. In some embodiments, R^{1A} is —S(O)₂R, —S(O)₂NR₂, —S(O)₂F, —S(O)R, —S(O)NR₂, or —S(O)(NR)R. In some embodiments, R^{1A} is —C(O)R, —C(O)OR, —C(O)NR₂, or —C(O)N(R)OR. In some embodiments, R^{1A} is —OC(O)R or —OC(O)NR₂. In some embodiments, R^{1A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{1A} is —P(O)R₂ or —P(O)(R)OR.

In some embodiments, R^{1A} is —OR, —OC(O)R, or —OC(O)NR₂. In some embodiments, R^{1A} is —SR, —S(O)₂R, —S(O)₂NR₂, —S(O)₂F, —S(O)R, —S(O)NR₂, or —S(O)(NR)R. In some embodiments, R^{1A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{1A} is —S(O)₂R, —S(O)₂NR₂, or —S(O)₂F. In some embodiments, R^{1A} is —S(O)R, —S(O)NR₂, or —S(O)(NR)R. In some embodiments, R^{1A} is —SR, —S(O)₂R, or —S(O)R. In some embodiments, R^{1A} is —S(O)₂NR₂, —S(O)NR₂, or —S(O)(NR)R. In some embodiments, R^{1A} is —S(O)₂NR₂ or —S(O)NR₂. In some embodiments, R^{1A} is —SR, —S(O)₂R, —S(O)₂NR₂, or —S(O)R.

In some embodiments, R^{1A} is —N(R)C(O)OR, —N(R)C(O)R, or —N(R)C(O)NR₂. In some embodiments, R^{1A} is —N(R)S(O)₂NR₂ or —N(R)S(O)₂R. In some embodiments, R^{1A} is —N(R)C(O)OR or —N(R)C(O)R. In some embodiments, R^{1A} is —N(R)C(O)NR₂ or —N(R)S(O)₂NR₂. In some embodiments, R^{1A} is —N(R)C(O)OR, —N(R)C(O)R, or —N(R)S(O)₂R.

In some embodiments, R^{1A} is —NR₂, —N(R)C(O)OR, —N(R)C(O)R, or —N(R)C(O)NR₂. In some embodiments, R^{1A} is —NR₂, —N(R)C(O)OR, or —N(R)C(O)R. In some embodiments, R^{1A} is —NR₂, —N(R)C(O)OR, —N(R)C(O)R, or —N(R)S(O)₂R.

In some embodiments, R^{1A} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is a C₁₋₆ aliphatic chain substituted by r¹ instances of R^{1C} In some embodiments, R^{1A} is phenyl substituted by r¹ instances of R^{1C}. In some embodiments, R^{1A} is naphthyl substituted by r¹ instances of R^{1C}. In some embodiments, R^{1A} is a 5-6 membered monocyclic heteroaryl ring having 1-4 heteroatoms independently selected from nitrogen, oxygen, and sulfur; wherein said ring is substituted by r¹ instances of R^{1C}. In some embodiments, R^{1A} is an 8-10 membered bicyclic heteroaryl ring having 1-4 heteroatoms independently selected from nitrogen, oxygen, and sulfur; wherein said ring is substituted by r¹ instances of R^{1C}. In some embodiments, R^{1A} is a 3-7 membered saturated or partially unsaturated monocyclic carbocyclic ring substituted by r¹ instances of R^{1C}. In some embodiments, R^{1A} is a 5-12 membered saturated or partially unsaturated bicyclic carbocyclic ring substituted by r¹ instances of R^{1C}. In some embodiments, R^{1A} is a 3-7 membered saturated or partially unsaturated monocyclic heterocyclic ring having 1-2 heteroatoms independently selected from nitrogen, oxygen, and sulfur; wherein said ring is substituted by r¹ instances of R^{1C}. In some embodiments, R^{1A} is a 7-12 membered saturated or partially unsaturated bicyclic heterocyclic ring having 1-4 heteroatoms independently selected from nitrogen, oxygen, and sulfur; wherein said ring is substituted by r¹ instances of R^{1C}.

In some embodiments, R^{1A} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r¹ instances of R^{1C}. In some embodiments, R^{1A} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is phenyl; naphthyl; a 3-7 membered saturated or partially unsaturated monocyclic carbocyclic ring; or a 5-12 membered saturated or partially unsaturated bicyclic carbocyclic ring; each of which is substituted by r¹ instances of R^{1C}. In some embodiments, R^{1A} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r¹ instances of R^{1C}. In some embodiments, R^{1A} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is phenyl or naphthyl; each of which is substituted by r¹ instances of R^{1C}. In some embodiments, R^{1A} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r¹ instances of R^{1C}. In some embodiments, R^{1A} is a 3-7 membered saturated or partially unsaturated monocyclic carbocyclic ring or a 5-12 membered saturated or partially unsaturated bicyclic carbocyclic ring; each of which is substituted by r¹ instances of R^{1C}. In some embodiments, R^{1A} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is phenyl or a 5-6 membered monocyclic heteroaryl ring having 1-4 heteroatoms independently selected from nitrogen, oxygen, and sulfur; each of which is substituted by r¹ instances of R^{1C}. In some embodiments, R^{1A} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by r¹ instances of R^{1C}. In some embodiments, R^{1A} is naphthyl or an 8-10 membered bicyclic heteroaryl ring having 1-4 heteroatoms independently selected from nitrogen, oxygen, and sulfur; each of which is substituted by r¹ instances of R^{1C}. In some embodiments, R^{1A} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is phenyl or a 3-7 membered saturated or partially unsaturated monocyclic carbocyclic ring; each of which is substituted by r¹ instances of R^{1C} In some embodiments, R^{1A} is naphthyl or a 5-12 membered saturated or partially unsaturated bicyclic carbocyclic ring; each of which is substituted by r¹ instances of R^{1C}. In some embodiments, R^{1A} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r¹ instances of R^{1C}. In some embodiments, R^{1A} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}. In some embodiments, R^{1A} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by r¹ instances of R^{1C}. In some embodiments, R^{1A} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r¹ instances of R^{1C}.

In some embodiments, R^{1A} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by r¹ instances of R^{1C}. In some embodiments, R^{1A} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by r¹ instances of R^{1C}. In some embodiments, R^{1A} is a C₁₋₆ aliphatic chain, phenyl, or a 3-7 membered saturated or partially unsaturated monocyclic carbocyclic ring; each of which is substituted by r¹ instances of R^{1C}.

In some embodiments, each R^{1A} is independently halogen, —CN, —OR, or a C₁₋₆ aliphatic chain substituted with r¹ halogens. In some embodiments, each R^{1A} is independently halogen, —CN, —OR, or a C₁₋₆ aliphatic chain substituted with 0-5 halogens. In some embodiments, each R^{1A} is independently halogen, —CN, —O—(C₁₋₆ aliphatic chain substituted with 0-5 halogens), or a C₁₋₆ aliphatic chain substituted with 0-5 halogens. In some embodiments, each R^{1A} is independently halogen or a C₁₋₆ aliphatic chain substituted with 0-5 halogens. In some embodiments, each R^{1A} is independently halogen or a C₁₋₆ aliphatic chain substituted with 0-4 halogens. In some embodiments, each R^{1A} is independently halogen or a C₁₋₆ aliphatic chain substituted with 0-3 halogens. In some embodiments, each R^{1A} is independently halogen or a C₁₋₃ aliphatic chain substituted with 0-3 halogens. In some embodiments, each R^{1A} is independently halogen or a C₁₋₃ aliphatic chain substituted with 0-2 halogens.

In some embodiments, each R^{1A} is independently a halogen selected from Br, Cl, and F. In some embodiments, each R^{1A} is independently a halogen selected from Cl and F. In some embodiments, R^{1A} is Cl. In some embodiments, R^{1A} is F.

In some embodiments, at least one R^{1A} is halogen. In some embodiments, at least two R^{1A} are halogen. In some embodiments, at least three R^{1A} are halogen. In some embodiments one instance of R^{1A} is Cl. In some embodiments two instances of R^{1A} are Cl. In some embodiments, one instance of R^{1A} is F. In some embodiments, two instances of R^{1A} are F. In some embodiments, one instance of R^{1A} is Cl, and one instance of R^{1A} is F. In some embodiments, two instances of R^{1A} are Cl, and one instance of R^{1A} is F. In some embodiments, one instance of R^{1A} is Cl, and two instances of R^{1A} are F.

In some embodiments, R^{1A} is a C₁₋₆ aliphatic chain substituted with 0-5 halogens. In some embodiments, R^{1A} is a C₁₋₆ aliphatic chain substituted with 0-4 halogens. In some embodiments, R^{1A} is a C₁₋₆ aliphatic chain substituted with 0-3 halogens. In some embodiments, R^{1A} is a C₁₋₃ aliphatic chain substituted with 0-3 halogens. In some embodiments, R^{1A} is a C₁₋₃ aliphatic chain substituted with 0-2 halogens.

In some embodiments, at least one R^{1A} is C₁₋₃ aliphatic optionally substituted with 1-3 halogen. In some embodiments, at least one R^{1A} is —O—C₁₋₃ aliphatic optionally substituted with 1-3 halogen.

In some embodiments, each R^{1A} is independently halogen, —OH, —OCH₃, or C₁₋₃ aliphatic optionally substituted with 1-3 halogen. In some embodiments, each R^{1A} is independently fluorine, chlorine, —OCH₃, or —CH₃. In some embodiments, R^{1A} is —OH. In some embodiments, R^{1A} is —CH₃. In some embodiments, R^{1A} is —OCH₃. In some embodiments, R^{1A} is —CF₃. In some embodiments, R^{1A} is —CHF₂.

In some embodiments, R^{1A} is selected from the groups depicted in the compounds in Table 1.

As defined generally above, each R^{2A} is independently R^{A} or R^{B} substituted by r² instances of R^{2C}. In some embodiments, each R^{2A} is R^{A}. In some embodiments, each R^{2A} is R^{B} substituted by r² instances of R^{2C}.

In some embodiments, R^{2A} is phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{2A} is substituted by r² instances of R^{2C}.

In some embodiments, R^{2A} is phenyl; naphthyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{2A} is substituted by r² instances of R^{2C}. In some embodiments, R^{2A} is phenyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{2A} is substituted by r² instances of R^{2C}. In some embodiments, R^{2A} is phenyl or an 8-10 membered bicyclic heteroaryl ring having 1-4 heteroatoms independently selected from nitrogen, oxygen, and sulfur; wherein R^{2A} is substituted by r² instances of R^{2C}.

In some embodiments, R^{2A} is phenyl; naphthyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{2A} is substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)O R, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{2A} is phenyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{2A} is substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{2A} is phenyl or an 8-10 membered bicyclic heteroaryl ring having 1-4 heteroatoms independently selected from nitrogen, oxygen, and sulfur; wherein R^{2A} is substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{2A} is phenyl substituted by r² instances of R^{2C}. In some embodiments, R^{2A} is phenyl substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{2A} is phenyl substituted by 1-3 instances of a group independently selected from halogen, —CN, —O-(optionally substituted C₁₋₆ aliphatic), and an optionally substituted C₁₋₆ aliphatic. In some embodiments, R^{2A} is phenyl substituted by 1-3 instances of a group independently selected from halogen and C₁₋₃ aliphatic optionally substituted with 1-3 halogen. In some embodiments, R^{2A} is phenyl substituted by 1-3 instances of a group independently selected from fluorine, chlorine, —CH₃, —CHF₂, and —CF₃.

In some embodiments, R^{2A} is phenyl substituted by 2 instances of a group independently selected from halogen, —CN, —O-(optionally substituted C₁₋₆ aliphatic), and an optionally substituted C₁₋₆ aliphatic. In some embodiments, R^{2A} is phenyl substituted by 2 instances of a group independently selected from halogen and C₁₋₃ aliphatic optionally substituted with 1-3 halogen. In some embodiments, R^{2A} is phenyl substituted by 2 instances of a group independently selected from fluorine, chlorine, —CH₃, —CHF₂, and —CF₃.

In some embodiments, R^{2A} is an 8-10 membered bicyclic heteroaryl ring having 1-4 heteroatoms independently selected from nitrogen, oxygen, and sulfur; wherein R^{2A} is substituted by r² instances of R^{2C}. In some embodiments, R^{2A} is an 8-10 membered bicyclic heteroaryl ring having 1-4 heteroatoms independently selected from nitrogen, oxygen, and sulfur; wherein R^{2A} is substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{2A} is an 8-10 membered bicyclic heteroaryl ring having 1-2 heteroatoms independently selected from nitrogen, oxygen, and sulfur; wherein R^{2A} is substituted by r² instances of R^{2C}. In some embodiments, R^{2A} is an 8-10 membered bicyclic heteroaryl ring having 1-2 heteroatoms independently selected from nitrogen, oxygen, and sulfur; wherein R^{2A} is substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂, and optionally substituted C₁₋₆ aliphatic.

In some embodiments, R^{2A} is an 8-10 membered bicyclic heteroaryl ring having 1-2 heteroatoms independently selected from nitrogen, oxygen, and sulfur; wherein R^{2A} is substituted by 0-2 instances of a group independently selected from halogen, —CN, —O-(optionally substituted C₁₋₆ aliphatic), and an optionally substituted C₁₋₆ aliphatic. In some embodiments, R^{2A} is an 8-10 membered bicyclic heteroaryl ring having 1-2 heteroatoms independently selected from nitrogen, oxygen, and sulfur; wherein R^{2A} is substituted by 0-2 instances of a group independently selected from halogen and C₁₋₃ aliphatic optionally substituted with 1-3 halogen. In some embodiments, R^{2A} is an 8-10 membered bicyclic heteroaryl ring having 1-2 heteroatoms independently selected from nitrogen, oxygen, and sulfur; wherein R^{2A} is substituted by 0-2 instances of a group independently selected from fluorine, chlorine, —CH₃, —CHF₂, and —CF₃.

In some embodiments, R^{2A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{2A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{2A} is oxo. In some embodiments, R^{2A} is halogen. In some embodiments, R^{2A} is —CN. In some embodiments, R^{2A} is —NO₂. In some embodiments, R^{2A} is —OR. In some embodiments, R^{2A} is —SR. In some embodiments, R^{2A} is —NR₂. In some embodiments, R^{2A} is —S(O)₂R. In some embodiments, R^{2A} is —S(O)₂NR₂. In some embodiments, R^{2A} is —S(O)₂F. In some embodiments, R^{2A} is —S(O)R. In some embodiments, R^{2A} is —S(O)NR₂. In some embodiments, R^{2A} is —S(O)(NR)R. In some embodiments, R^{2A} is —C(O)R. In some embodiments, R^{2A} is —C(O)OR. In some embodiments, R^{2A} is —C(O)NR₂. In some embodiments, R^{2A} is —C(O)N(R)OR. In some embodiments, R^{2A} is —OC(O)R. In some embodiments, R^{2A} is —OC(O)NR₂. In some embodiments, R^{2A} is —N(R)C(O)OR. In some embodiments, R^{2A} is —N(R)C(O)R. In some embodiments, R^{2A} is —N(R)C(O)NR₂. In some embodiments, R^{2A} is —N(R)C(NR)NR₂. In some embodiments, R^{2A} is —N(R)S(O)₂NR₂. In some embodiments, R^{2A} is —N(R)S(O)₂R. In some embodiments, R^{2A} is —P(O)R₂. In some embodiments, R^{2A} is —P(O)(R)OR. In some embodiments, R^{2A} is —B(OR)₂. In some embodiments, R^{2A} is deuterium.

In some embodiments, R^{2A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R², —P(O)(R)OR, or —B(OR)₂.

In some embodiments, R^{2A} is halogen, —CN, or —NO₂. In some embodiments, R^{2A} is —OR, —SR, or —NR₂. In some embodiments, R^{2A} is —S(O)₂R, —S(O)₂NR₂, —S(O)₂F, —S(O)R, —S(O)NR₂, or —S(O)(NR)R. In some embodiments, R^{2A} is —C(O)R, —C(O)OR, —C(O)NR₂, or —C(O)N(R)OR. In some embodiments, R^{2A} is —OC(O)R or —OC(O)NR₂. In some embodiments, R^{2A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{2A} is —P(O)R₂ or —P(O)(R)OR.

In some embodiments, R^{2A} is —OR, —OC(O)R, or —OC(O)NR₂. In some embodiments, R^{2A} is —SR, —S(O)₂R, —S(O)₂NR₂, —S(O)₂F, —S(O)R, —S(O)NR₂, or —S(O)(NR)R. In some embodiments, R^{2A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{2A} is —S(O)₂R, —S(O)₂NR₂, or —S(O)₂F. In some embodiments, R^{2A} is —S(O)R, —S(O)NR₂, or —S(O)(NR)R. In some embodiments, R^{2A} is —SR, —S(O)₂R, or —S(O)R. In some embodiments, R^{2A} is —S(O)₂NR₂, —S(O)NR₂, or —S(O)(NR)R. In some embodiments, R^{2A} is —S(O)₂NR₂ or —S(O)NR₂. In some embodiments, R^{2A} is —SR, —S(O)₂R, —S(O)₂NR₂, or —S(O)R.

In some embodiments, R^{2A} is —N(R)C(O)OR, —N(R)C(O)R, or —N(R)C(O)NR₂. In some embodiments, R^{2A} is —N(R)S(O)₂NR₂ or —N(R)S(O)₂R. In some embodiments, R^{2A} is —N(R)C(O)OR or —N(R)C(O)R. In some embodiments, R^{2A} is —N(R)C(O)NR₂ or —N(R)S(O)₂NR₂. In some embodiments, R^{2A} is —N(R)C(O)OR, —N(R)C(O)R, or —N(R)S(O)₂R.

In some embodiments, R^{2A} is —NR₂, —N(R)C(O)OR, —N(R)C(O)R, or —N(R)C(O)NR₂. In some embodiments, R^{2A} is —NR₂, —N(R)C(O)OR, or —N(R)C(O)R. In some embodiments, R^{2A} is —NR₂, —N(R)C(O)OR, —N(R)C(O)R, or —N(R)S(O)₂R.

In some embodiments, R^{2A} is a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is a C₁₋₆ aliphatic chain substituted by r² instances of R^{2C}. In some embodiments, R^{2A} is phenyl substituted by r² instances of R^{2C}. In some embodiments, R^{2A} is naphthyl substituted by r² instances of R^{2C}. In some embodiments, R^{2A} is cubanyl substituted by r² instances of R^{2C}. In some embodiments, R^{2A} is adamantyl substituted by r² instances of R^{2C}. In some embodiments, R^{2A} is a 5-6 membered monocyclic heteroaryl ring having 1-4 heteroatoms independently selected from nitrogen, oxygen, and sulfur; wherein said ring is substituted by r² instances of R^{2C}. In some embodiments, R^{2A} is an 8-10 membered bicyclic heteroaryl ring having 1-4 heteroatoms independently selected from nitrogen, oxygen, and sulfur; wherein said ring is substituted by r² instances of R^{2C}. In some embodiments, R^{2A} is a 3-7 membered saturated or partially unsaturated monocyclic carbocyclic ring substituted by r² instances of R^{2C}. In some embodiments, R^{2A} is a 5-12 membered saturated or partially unsaturated bicyclic carbocyclic ring substituted by r² instances of R^{2C}. In some embodiments, R^{2A} is a 3-7 membered saturated or partially unsaturated monocyclic heterocyclic ring having 1-2 heteroatoms independently selected from nitrogen, oxygen, and sulfur; wherein said ring is substituted by r² instances of R^{2C}. In some embodiments, R^{2A} is a 7-12 membered saturated or partially unsaturated bicyclic heterocyclic ring having 1-4 heteroatoms independently selected from nitrogen, oxygen, and sulfur; wherein said ring is substituted by r² instances of R^{2C}.

In some embodiments, R^{2A} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r² instances of R^{2C}. In some embodiments, R^{2A} is cubanyl; adamantyl; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is phenyl; naphthyl; cubanyl; adamantyl; a 3-7 membered saturated or partially unsaturated monocyclic carbocyclic ring; or a 5-12 membered saturated or partially unsaturated bicyclic carbocyclic ring; each of which is substituted by r² instances of R^{2C}. In some embodiments, R^{2A} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r² instances of R^{2C}. In some embodiments, R^{2A} is naphthyl; cubanyl; adamantyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is phenyl or naphthyl; each of which is substituted by r² instances of R^{2C}. In some embodiments, R^{2A} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r² instances of R^{2C}. In some embodiments, R^{2A} is a 3-7 membered saturated or partially unsaturated monocyclic carbocyclic ring or a 5-12 membered saturated or partially unsaturated bicyclic carbocyclic ring; each of which is substituted by r² instances of R^{2C}. In some embodiments, R² is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is phenyl or a 5-6 membered monocyclic heteroaryl ring having 1-4 heteroatoms independently selected from nitrogen, oxygen, and sulfur; each of which is substituted by r² instances of R^{2C}. In some embodiments, R^{2A} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by r² instances of R^{2C}. In some embodiments, R^{2A} is naphthyl or an 8-10 membered bicyclic heteroaryl ring having 1-4 heteroatoms independently selected from nitrogen, oxygen, and sulfur; each of which is substituted by r² instances of R^{2C}. In some embodiments, R^{2A} is cubanyl; adamantyl; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is phenyl or a 3-7 membered saturated or partially unsaturated monocyclic carbocyclic ring; each of which is substituted by r² instances of R^{2C}. In some embodiments, R^{2A} is naphthyl; cubanyl; adamantyl; or a 5-12 membered saturated or partially unsaturated bicyclic carbocyclic ring; each of which is substituted by r² instances of R^{2C}. In some embodiments, R^{2A} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r² instances of R^{2C}. In some embodiments, R^{2A} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is a C₁₋₆ aliphatic chain; cubanyl; adamantyl; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}. In some embodiments, R^{2A} is a C₁₋₆ aliphatic chain; phenyl; naphthyl; cubanyl; adamantyl; a 3-7 membered saturated or partially unsaturated monocyclic carbocyclic ring; or a 5-12 membered saturated or partially unsaturated bicyclic carbocyclic ring; each of which is substituted by r² instances of R^{2C}. In some embodiments, R^{2A} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r² instances of R^{2C}.

In some embodiments, R^{2A} is a C₁₋₆ aliphatic chain, cubanyl, adamantyl, a 3-7 membered saturated or partially unsaturated monocyclic carbocyclic ring, or a 5-12 membered saturated or partially unsaturated bicyclic carbocyclic ring; each of which is substituted by r² instances of R^{2C}. In some embodiments, R^{2A} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by r² instances of R^{2C}. In some embodiments, R^{2A} is a C₁₋₆ aliphatic chain, phenyl, or a 3-7 membered saturated or partially unsaturated monocyclic carbocyclic ring; each of which is substituted by r² instances of R^{2C}.

In some embodiments, R^{2A} is selected from the groups depicted in the compounds in Table 1.

As defined generally above, each R^{TA} is independently R^{A} or R^{B} substituted with r³ instances of R^{TC}. In some embodiments, each R^{T} is independently R^{A}. In some embodiments, each R^{T} is independently R^{B} substituted with r³ instances of R^{TC}.

In some embodiments, R^{TA} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{TA} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{TA} is oxo. In some embodiments, R^{TA} is halogen. In some embodiments, R^{TA} is —CN. In some embodiments, R^{TA} is —NO₂. In some embodiments, R^{TA} is —OR. In some embodiments, R^{TA} is —SR. In some embodiments, R^{TA} is —NR₂. In some embodiments, R^{TA} is —S(O)₂R. In some embodiments, R^{TA} is —S(O)₂NR₂. In some embodiments, R^{TA} is —S(O)₂F. In some embodiments, R^{TA} is —S(O)R. In some embodiments, R^{TA} is —S(O)NR₂. In some embodiments, R^{TA} is —S(O)(NR)R. In some embodiments, R^{TA} is —C(O)R. In some embodiments, R^{TA} is —C(O)OR. In some embodiments, R^{TA} is —C(O)NR₂. In some embodiments, R^{TA} is —C(O)N(R)OR. In some embodiments, R^{TA} is —OC(O)R. In some embodiments, R^{TA} is —OC(O)NR₂. In some embodiments, R^{TA} is —N(R)C(O)OR. In some embodiments, R^{TA} is —N(R)C(O)R. In some embodiments, R^{TA} is —N(R)C(O)NR₂. In some embodiments, R^{TA} is —N(R)C(NR)NR₂. In some embodiments, R^{TA} is —N(R)S(O)₂NR₂. In some embodiments, R^{TA} is —N(R)S(O)₂R. In some embodiments, R^{TA} is —P(O)R₂. In some embodiments, R^{TA} is —P(O)(R)OR. In some embodiments, R^{TA} is —B(OR)₂. In some embodiments, R^{TA} is deuterium.

In some embodiments, R^{TA} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{TA} is halogen, —CN, or —NO₂. In some embodiments, R^{TA} is —OR, —SR, or —NR₂. In some embodiments, R^{TA} is —S(O)₂R, —S(O)₂NR₂, —S(O)₂F, —S(O)R, —S(O)NR₂, or —S(O)(NR)R. In some embodiments, R^{TA} is —C(O)R, —C(O)OR, —C(O)NR₂, or —C(O)N(R)OR. In some embodiments, R^{TA} is —OC(O)R or —OC(O)NR₂. In some embodiments, R^{TA} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{TA} is —P(O)R₂ or —P(O)(R)OR.

In some embodiments, R^{TA} is —OR, —OC(O)R, or —OC(O)NR₂. In some embodiments, R^{TA} is —SR, —S(O)₂R, —S(O)₂NR₂, —S(O)₂F, —S(O)R, —S(O)NR₂, or —S(O)(NR)R. In some embodiments, R^{TA} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{TA} is —S(O)₂R, —S(O)₂NR₂, or —S(O)₂F. In some embodiments, R^{TA} is —S(O)R, —S(O)NR₂, or —S(O)(NR)R. In some embodiments, R^{TA} is —SR, —S(O)₂R, or —S(O)R. In some embodiments, R^{TA} is —S(O)₂NR₂, —S(O)NR₂, or —S(O)(NR)R. In some embodiments, R^{TA} is —S(O)₂NR₂ or —S(O)NR₂. In some embodiments, R^{TA} is —SR, —S(O)₂R, —S(O)₂NR₂, or —S(O)R.

In some embodiments, R^{TA} is —N(R)C(O)OR, —N(R)C(O)R, or —N(R)C(O)NR₂. In some embodiments, R^{TA} is —N(R)S(O)₂NR₂ or —N(R)S(O)₂R. In some embodiments, R^{TA} is —N(R)C(O)OR or —N(R)C(O)R. In some embodiments, R^{TA} is —N(R)C(O)NR₂ or —N(R)S(O)₂NR₂. In some embodiments, R^{TA} is —N(R)C(O)OR, —N(R)C(O)R, or —N(R)S(O)₂R.

In some embodiments, R^{TA} is —NR₂, —N(R)C(O)OR, —N(R)C(O)R, or —N(R)C(O)NR₂. In some embodiments, R^{TA} is —NR₂, —N(R)C(O)OR, or —N(R)C(O)R. In some embodiments, R^{TA} is —NR₂, —N(R)C(O)OR, —N(R)C(O)R, or —N(R)S(O)₂R.

In some embodiments, R^{TA} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r³ instances of R^{TC}.

In some embodiments, R^{TA} is a C₁₋₆ aliphatic chain substituted by r³ instances of R^{TC} In some embodiments, R^{TA} is phenyl substituted by r³ instances of R^{TC}. In some embodiments, R^{TA} is naphthyl substituted by r³ instances of R^{TC}. In some embodiments, R^{TA} is a 5-6 membered monocyclic heteroaryl ring having 1-4 heteroatoms independently selected from nitrogen, oxygen, and sulfur; wherein said ring is substituted by r³ instances of R^{TC}. In some embodiments, R^{TA} is an 8-10 membered bicyclic heteroaryl ring having 1-4 heteroatoms independently selected from nitrogen, oxygen, and sulfur; wherein said ring is substituted by r³ instances of R^{TC}. In some embodiments, R^{TA} is a 3-7 membered saturated or partially unsaturated monocyclic carbocyclic ring substituted by r³ instances of R^{TC}. In some embodiments, R^{TA} is a 5-12 membered saturated or partially unsaturated bicyclic carbocyclic ring substituted by r³ instances of R^{TC}. In some embodiments, R^{TA} is a 3-7 membered saturated or partially unsaturated monocyclic heterocyclic ring having 1-2 heteroatoms independently selected from nitrogen, oxygen, and sulfur; wherein said ring is substituted by r³ instances of R^{TC}. In some embodiments, R^{TA} is a 7-12 membered saturated or partially unsaturated bicyclic heterocyclic ring having 1-4 heteroatoms independently selected from nitrogen, oxygen, and sulfur; wherein said ring is substituted by r³ instances of R^{TC}.

In some embodiments, RTA is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r³ instances of R^{TC}.

In some embodiments, R^{TA} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r³ instances of R^{TC}. In some embodiments, R^{TA} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r³ instances of R^{TC}.

In some embodiments, R^{TA} is phenyl; naphthyl; a 3-7 membered saturated or partially unsaturated monocyclic carbocyclic ring; or a 5-12 membered saturated or partially unsaturated bicyclic carbocyclic ring; each of which is substituted by r³ instances of R^{TC}. In some embodiments, R^{TA} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r³ instances of R^{TC}.

In some embodiments, R^{TA} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r³ instances of R^{TC}. In some embodiments, R^{TA} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r³ instances of R^{TC}.

In some embodiments, R^{TA} is phenyl or naphthyl; each of which is substituted by r³ instances of R^{TC}. In some embodiments, R^{TA} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r³ instances of R^{TC}. In some embodiments, R^{TA} is a 3-7 membered saturated or partially unsaturated monocyclic carbocyclic ring or a 5-12 membered saturated or partially unsaturated bicyclic carbocyclic ring; each of which is substituted by r³ instances of R^{TC}. In some embodiments, R^{TA} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r³ instances of R^{TC}.

In some embodiments, R^{TA} is phenyl or a 5-6 membered monocyclic heteroaryl ring having 1-4 heteroatoms independently selected from nitrogen, oxygen, and sulfur; each of which is substituted by r³ instances of R^{TC}. In some embodiments, R^{TA} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by r³ instances of R^{TC}. In some embodiments, R^{TA} is naphthyl or an 8-10 membered bicyclic heteroaryl ring having 1-4 heteroatoms independently selected from nitrogen, oxygen, and sulfur; each of which is substituted by r³ instances of R^{TC}. In some embodiments, R^{TA} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by r³ instances of R^{TC}.

In some embodiments, R^{TA} is phenyl or a 3-7 membered saturated or partially unsaturated monocyclic carbocyclic ring; each of which is substituted by r³ instances of R^{TC} In some embodiments, R^{TA} is naphthyl or a 5-12 membered saturated or partially unsaturated bicyclic carbocyclic ring; each of which is substituted by r³ instances of R^{TC}. In some embodiments, R^{TA} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r³ instances of R^{TC}. In some embodiments, R^{TA} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r³ instances of R^{TC}.

In some embodiments, R^{TA} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r³ instances of R^{TC}. In some embodiments, R^{TA} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by r³ instances of R^{TC}. In some embodiments, R^{TA} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r³ instances of R^{TC}.

In some embodiments, R^{TA} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by r³ instances of R^{TC} In some embodiments, R^{TA} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by r³ instances of RC. In some embodiments, R^{TA} is a C₁₋₆ aliphatic chain, phenyl, or a 3-7 membered saturated or partially unsaturated monocyclic carbocyclic ring; each of which is substituted by r³ instances of R^{TC}.

In some embodiments, R^{TA} is selected from the groups depicted in the compounds in Table 1.

As defined generally above, each R^{L} is independently R^{A} or R^{B} substituted by r⁴ instances of R^{LC}. In some embodiments, each R^{L} is independently R^{A}. In some embodiments, each R^{L} is independently R^{B} substituted by r⁴ instances of R^{LC}.

In some embodiments, R^{L} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{L} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R², —P(O)(R)OR, or —B(OR)₂.

In some embodiments, R^{L} is oxo. In some embodiments, R^{L} is halogen. In some embodiments, R^{L} is —CN. In some embodiments, R^{L} is —NO₂. In some embodiments, R^{L} is —OR. In some embodiments, R^{L} is —SR. In some embodiments, R^{L} is —NR₂. In some embodiments, R^{L} is —S(O)₂R. In some embodiments, R^{L} is —S(O)₂NR₂. In some embodiments, R^{L} is —S(O)₂F. In some embodiments, R^{L} is —S(O)R. In some embodiments, R^{L} is —S(O)NR₂. In some embodiments, R^{L} is —S(O)(NR)R. In some embodiments, R^{L} is —C(O)R. In some embodiments, R^{L} is —C(O)OR. In some embodiments, R^{L} is —C(O)NR₂. In some embodiments, R^{L} is —C(O)N(R)OR. In some embodiments, R^{L} is —OC(O)R. In some embodiments, R^{L} is —OC(O)NR₂. In some embodiments, R^{L} is —N(R)C(O)OR. In some embodiments, R^{L} is —N(R)C(O)R. In some embodiments, R^{L} is —N(R)C(O)NR₂. In some embodiments, R^{L} is —N(R)C(NR)NR₂. In some embodiments, R^{L} is —N(R)S(O)₂NR₂. In some embodiments, R^{L} is —N(R)S(O)₂R. In some embodiments, R^{L} is —P(O)R₂. In some embodiments, R^{L} is —P(O)(R)OR. In some embodiments, R^{L} is —B(OR)₂. In some embodiments, R^{L} is deuterium.

In some embodiments, R^{L} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{L} is halogen, —CN, or —NO₂. In some embodiments, R^{L} is —OR, —SR, or —NR₂. In some embodiments, R^{L} is —S(O)₂R, —S(O)₂NR₂, —S(O)₂F, —S(O)R, —S(O)NR₂, or —S(O)(NR)R. In some embodiments, R^{L} is —C(O)R, —C(O)OR, —C(O)NR₂, or —C(O)N(R)OR. In some embodiments, R^{L} is —OC(O)R or —OC(O)NR₂. In some embodiments, R^{L} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{L} is —P(O)R₂ or —P(O)(R)OR.

In some embodiments, R^{L} is —OR, —OC(O)R, or —OC(O)NR₂. In some embodiments, R^{L} is —SR, —S(O)₂R, —S(O)₂NR₂, —S(O)₂F, —S(O)R, —S(O)NR₂, or —S(O)(NR)R. In some embodiments, R^{L} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{L} is —S(O)₂R, —S(O)₂NR₂, or —S(O)₂F. In some embodiments, R^{L} is —S(O)R, —S(O)NR₂, or —S(O)(NR)R. In some embodiments, R^{L} is —SR, —S(O)₂R, or —S(O)R. In some embodiments, R^{L} is —S(O)₂NR₂, —S(O)NR₂, or —S(O)(NR)R. In some embodiments, R^{L} is —S(O)₂NR₂ or —S(O)NR₂. In some embodiments, R^{L} is —SR, —S(O)₂R, —S(O)₂NR₂, or —S(O)R.

In some embodiments, R^{L} is —N(R)C(O)OR, —N(R)C(O)R, or —N(R)C(O)NR₂. In some embodiments, R^{L} is —N(R)S(O)₂NR₂ or —N(R)S(O)₂R. In some embodiments, R^{L} is —N(R)C(O)OR or —N(R)C(O)R. In some embodiments, R^{L} is —N(R)C(O)NR₂ or —N(R)S(O)₂NR₂. In some embodiments, R^{L} is —N(R)C(O)OR, —N(R)C(O)R, or —N(R)S(O)₂R.

In some embodiments, R^{L} is —NR₂, —N(R)C(O)OR, —N(R)C(O)R, or —N(R)C(O)NR₂. In some embodiments, R^{L} is —NR₂, —N(R)C(O)OR, or —N(R)C(O)R. In some embodiments, R^{L} is —NR₂, —N(R)C(O)OR, —N(R)C(O)R, or —N(R)S(O)₂R.

In some embodiments, R^{L} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁴ instances of R^{LC}.

In some embodiments, R^{L} is a C₁₋₆ aliphatic chain substituted by r⁴ instances of R^{LC} In some embodiments, R^{L} is phenyl substituted by r⁴ instances of R^{LC}. In some embodiments, R^{L} is naphthyl substituted by r⁴ instances of R^{LC}. In some embodiments, R^{L} is a 5-6 membered monocyclic heteroaryl ring having 1-4 heteroatoms independently selected from nitrogen, oxygen, and sulfur; wherein said ring is substituted by r⁴ instances of R^{LC} In some embodiments, R^{L} is an 8-10 membered bicyclic heteroaryl ring having 1-4 heteroatoms independently selected from nitrogen, oxygen, and sulfur; wherein said ring is substituted by r⁴ instances of R^{LC}. In some embodiments, R^{L} is a 3-7 membered saturated or partially unsaturated monocyclic carbocyclic ring substituted by r⁴ instances of R^{LC}. In some embodiments, R^{L} is a 5-12 membered saturated or partially unsaturated bicyclic carbocyclic ring substituted by r⁴ instances of R^{LC}. Tn some embodiments, R^{L} is a 3-7 membered saturated or partially unsaturated monocyclic heterocyclic ring having 1-2 heteroatoms independently selected from nitrogen, oxygen, and sulfur; wherein said ring is substituted by r⁴ instances of R^{LC}. In some embodiments, R^{L} is a 7-12 membered saturated or partially unsaturated bicyclic heterocyclic ring having 1-4 heteroatoms independently selected from nitrogen, oxygen, and sulfur; wherein said ring is substituted by r⁴ instances of R^{LC}.

In some embodiments, R^{L} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁴ instances of R^{LC}.

In some embodiments, R^{L} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r⁴ instances of R^{LC}. In some embodiments, R^{L} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁴ instances of R^{LC}.

In some embodiments, R^{L} is phenyl; naphthyl; a 3-7 membered saturated or partially unsaturated monocyclic carbocyclic ring; or a 5-12 membered saturated or partially unsaturated bicyclic carbocyclic ring; each of which is substituted by r⁴ instances of R^{LC}. In some embodiments, R^{L} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁴ instances of R^{LC}.

In some embodiments, R^{L} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r⁴ instances of R^{LC}. In some embodiments, R^{L} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r⁴ instances of R^{LC}.

In some embodiments, R^{L} is phenyl or naphthyl; each of which is substituted by r⁴ instances of R^{LC}. In some embodiments, R^{L} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r⁴ instances of R^{LC}. In some embodiments, R^{L} is a 3-7 membered saturated or partially unsaturated monocyclic carbocyclic ring or a 5-12 membered saturated or partially unsaturated bicyclic carbocyclic ring; each of which is substituted by r⁴ instances of R^{LC}. In some embodiments, R^{L} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁴ instances of R^{LC}.

In some embodiments, R^{L} is phenyl or a 5-6 membered monocyclic heteroaryl ring having 1-4 heteroatoms independently selected from nitrogen, oxygen, and sulfur; each of which is substituted by r⁴ instances of R^{LC}. In some embodiments, R^{L} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by r⁴ instances of R^{LC}. In some embodiments, R^{L} is naphthyl or an 8-10 membered bicyclic heteroaryl ring having 1-4 heteroatoms independently selected from nitrogen, oxygen, and sulfur; each of which is substituted by r⁴ instances of R^{LC}. In some embodiments, R^{L} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by r⁴ instances of R^{LC}.

In some embodiments, R^{L} is phenyl or a 3-7 membered saturated or partially unsaturated monocyclic carbocyclic ring; each of which is substituted by r⁴ instances of R^{LC} In some embodiments, R^{L} is naphthyl or a 5-12 membered saturated or partially unsaturated bicyclic carbocyclic ring; each of which is substituted by r⁴ instances of R^{LC}. In some embodiments, R^{L} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r⁴ instances of R^{LC}. In some embodiments, R^{L} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁴ instances of R^{LC}.

In some embodiments, R^{L} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁴ instances of R^{LC}. In some embodiments, R^{L} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by r⁴ instances of R^{LC}. In some embodiments, R^{L} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r⁴ instances of R^{LC}.

In some embodiments, R^{L} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by r⁴ instances of R^{LC} In some embodiments, R^{L} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by r⁴ instances of R^{LC} In some embodiments, R^{L} is a C₁₋₆ aliphatic chain, phenyl, or a 3-7 membered saturated or partially unsaturated monocyclic carbocyclic ring; each of which is substituted by r⁴ instances of R^{LC}.

In some embodiments, R^{L} is selected from the groups depicted in the compounds in Table 1.

As defined generally above, each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{A} is independently oxo, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{A} is oxo. In some embodiments, R^{A} is halogen. In some embodiments, R^{A} is —CN. In some embodiments, R^{A} is —NO₂. In some embodiments, R^{A} is —OR. In some embodiments, R^{A} is —SF₅. In some embodiments, R^{A} is —SR. In some embodiments, R^{A} is —NR₂. In some embodiments, R^{A} is —S(O)₂R. In some embodiments, R^{A} is —S(O)₂NR₂. In some embodiments, R^{A} is —S(O)₂F. In some embodiments, R^{A} is —S(O)R. In some embodiments, R^{A} is —S(O)NR₂. In some embodiments, R^{A} is —S(O)(NR)R. In some embodiments, R^{A} is —C(O)R. In some embodiments, R^{A} is —C(O)OR. In some embodiments, R^{A} is —C(O)NR₂. In some embodiments, R^{A} is —C(O)N(R)OR. In some embodiments, R^{A} is —OC(O)R. In some embodiments, R^{A} is —OC(O)NR₂. In some embodiments, R^{A} is —N(R)C(O)OR. In some embodiments, R^{A} is —N(R)C(O)R. In some embodiments, R^{A} is —N(R)C(O)NR₂. In some embodiments, R^{A} is —N(R)C(NR)NR₂. In some embodiments, R^{A} is —N(R)S(O)₂NR₂. In some embodiments, R^{A} is —N(R)S(O)₂R. In some embodiments, R^{A} is —P(O)R₂. In some embodiments, R^{A} is —P(O)(R)OR. In some embodiments, R^{A} is —B(OR)₂. In some embodiments, R^{A} is deuterium.

In some embodiments, R^{A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{A} is halogen, —CN, or —NO₂. In some embodiments, R^{A} is —OR, —SR, or —NR₂. In some embodiments, R^{A} is —S(O)₂R, —S(O)₂NR₂, —S(O)₂F, —S(O)R, —S(O)NR₂, or —S(O)(NR)R. In some embodiments, R^{A} is —C(O)R, —C(O)OR, —C(O)NR₂, or —C(O)N(R)OR. In some embodiments, R^{A} is —OC(O)R or —OC(O)NR₂. In some embodiments, R^{A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{A} is —P(O)R₂ or —P(O)(R)OR.

In some embodiments, R^{A} is —OR, —OC(O)R, or —OC(O)NR₂. In some embodiments, R^{A} is —SR, —S(O)₂R, —S(O)₂NR₂, —S(O)₂F, —S(O)R, —S(O)NR₂, or —S(O)(NR)R. In some embodiments, R^{A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{A} is —S(O)₂R, —S(O)₂NR₂, or —S(O)₂F. In some embodiments, R^{A} is —S(O)R, —S(O)NR₂, or —S(O)(NR)R. In some embodiments, R^{A} is —SR, —S(O)₂R, or —S(O)R. In some embodiments, R^{A} is —S(O)₂NR₂, —S(O)NR₂, or —S(O)(NR)R. In some embodiments, R^{A} is —S(O)₂NR₂ or —S(O)NR₂. In some embodiments, R^{A} is —SR, —S(O)₂R, —S(O)₂NR₂, or —S(O)R.

In some embodiments, R^{A} is —N(R)C(O)OR, —N(R)C(O)R, or —N(R)C(O)NR₂. In some embodiments, R^{A} is —N(R)S(O)₂NR₂ or —N(R)S(O)₂R. In some embodiments, R^{A} is —N(R)C(O)OR or —N(R)C(O)R. In some embodiments, R^{A} is —N(R)C(O)NR₂ or —N(R)S(O)₂NR₂. In some embodiments, R^{A} is —N(R)C(O)OR, —N(R)C(O)R, or —N(R)S(O)₂R.

In some embodiments, R^{A} is —NR₂, —N(R)C(O)OR, —N(R)C(O)R, or —N(R)C(O)NR₂. In some embodiments, R^{A} is —NR₂, —N(R)C(O)OR, or —N(R)C(O)R. In some embodiments, R^{A} is —NR₂, —N(R)C(O)OR, —N(R)C(O)R, or —N(R)S(O)₂R.

In some embodiments, R^{A} is selected from the groups depicted in the compounds in Table 1.

As defined generally above, each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is a C₁₋₆ aliphatic chain. In some embodiments, R^{B} is phenyl. In some embodiments, R^{B} is naphthyl. In some embodiments, R^{B} is cubanyl. In some embodiments, R^{B} is adamantyl. In some embodiments, R^{B} is a 5-6 membered monocyclic heteroaryl ring having 1-4 heteroatoms independently selected from nitrogen, oxygen, and sulfur. In some embodiments, R^{B} is an 8-10 membered bicyclic heteroaryl ring having 1-4 heteroatoms independently selected from nitrogen, oxygen, and sulfur. In some embodiments, R^{B} is a 3-7 membered saturated or partially unsaturated monocyclic carbocyclic ring. In some embodiments, R^{B} is a 5-12 membered saturated or partially unsaturated bicyclic carbocyclic ring. In some embodiments, R^{B} is a 3-7 membered saturated or partially unsaturated monocyclic heterocyclic ring having 1-2 heteroatoms independently selected from nitrogen, oxygen, and sulfur. In some embodiments, R^{B} is a 7-12 membered saturated or partially unsaturated bicyclic heterocyclic ring having 1-4 heteroatoms independently selected from nitrogen, oxygen, and sulfur.

In some embodiments, R^{B} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur. In some embodiments, R^{B} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is phenyl; naphthyl; a 3-7 membered saturated or partially unsaturated monocyclic carbocyclic ring; or a 5-12 membered saturated or partially unsaturated bicyclic carbocyclic ring. In some embodiments, R^{B} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur. In some embodiments, R^{B} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur.

In some embodiments, R^{B} is phenyl or naphthyl. In some embodiments, R^{B} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur. In some embodiments, R^{B} is a 3-7 membered saturated or partially unsaturated monocyclic carbocyclic ring or a 5-12 membered saturated or partially unsaturated bicyclic carbocyclic ring. In some embodiments, R^{B} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is phenyl or a 5-6 membered monocyclic heteroaryl ring having 1-4 heteroatoms independently selected from nitrogen, oxygen, and sulfur. In some embodiments, R^{B} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur. In some embodiments, R^{B} is naphthyl or an 8-10 membered bicyclic heteroaryl ring having 1-4 heteroatoms independently selected from nitrogen, oxygen, and sulfur. In some embodiments, R^{B} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur.

In some embodiments, R^{B} is phenyl or a 3-7 membered saturated or partially unsaturated monocyclic carbocyclic ring. In some embodiments, R^{B} is naphthyl or a 5-12 membered saturated or partially unsaturated bicyclic carbocyclic ring. In some embodiments, R^{B} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur. In some embodiments, R^{B} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur. In some embodiments, R^{B} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring. In some embodiments, R^{B} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur.

In some embodiments, R^{B} is a C₁₋₆ aliphatic chain, a 3-7 membered saturated or partially unsaturated monocyclic carbocyclic ring, or a 5-12 membered saturated or partially unsaturated bicyclic carbocyclic ring. In some embodiments, R^{B} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur. In some embodiments, R^{B} is a C₁₋₆ aliphatic chain, phenyl, or a 3-7 membered saturated or partially unsaturated monocyclic carbocyclic ring.

In some embodiments, R^{B} is selected from the groups depicted in the compounds in Table 1.

As defined generally above, each instance of R^{1C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{1C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{1C} is oxo. In some embodiments, R^{1C} is deuterium. In some embodiments, each instance of R^{1C} is independently halogen. In some embodiments, R^{1C} is —CN. In some embodiments, R^{1C} is —NO₂. In some embodiments, R^{1C} is —OR. In some embodiments, R^{1C} is —SR. In some embodiments, R^{1C} is —NR₂. In some embodiments, R^{1C} is —S(O)₂R. In some embodiments, R^{1C} is —S(O)₂NR₂. In some embodiments, R^{1C} is —S(O)₂F. In some embodiments, R^{1C} is —S(O)R. In some embodiments, R^{1C} is —S(O)NR₂. In some embodiments, R^{1C} is —S(O)(NR)R. In some embodiments, R^{1C} is —C(O)R. In some embodiments, R^{1C} is —C(O)OR. In some embodiments, R^{1C} is —C(O)NR₂. In some embodiments, R^{1C} is —C(O)N(R)OR. In some embodiments, R^{1C} is —OC(O)R. In some embodiments, R^{1C} is —OC(O)NR₂. In some embodiments, R^{1C} is —N(R)C(O)OR. In some embodiments, R^{1C} is —N(R)C(O)R. In some embodiments, R^{1C} is —N(R)C(O)NR₂. In some embodiments, R^{1C} is —N(R)C(NR)NR₂. In some embodiments, R^{1C} is —N(R)S(O)₂NR₂. In some embodiments, R^{1C} is —N(R)S(O)₂R. In some embodiments, R^{1C} is —P(O)R₂. In some embodiments, R^{1C} is —P(O)(R)OR. In some embodiments, R^{1C} is —B(OR)₂.

In some embodiments, each instance of R^{1C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{1C} is independently halogen, —CN, or —NO₂. In some embodiments, each instance of R^{1C} is independently —OR, —SR, or —NR₂. In some embodiments, each instance of R^{1C} is independently —S(O)₂R, —S(O)₂NR₂, —S(O)₂F, —S(O)R, —S(O)NR₂, or —S(O)(NR)R. In some embodiments, each instance of R^{1C} is independently —C(O)R, —C(O)OR, —C(O)NR₂, or —C(O)N(R)OR. In some embodiments, each instance of R^{1C} is independently —OC(O)R or —OC(O)NR₂. In some embodiments, each instance of R^{1C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{1C} is independently —P(O)R₂ or —P(O)(R)OR.

In some embodiments, each instance of R^{1C} is independently —OR, —OC(O)R, or —OC(O)NR₂. In some embodiments, each instance of R^{1C} is independently —SR, —S(O)₂R, —S(O)₂NR₂, —S(O)₂F, —S(O)R, —S(O)NR₂, or —S(O)(NR)R. In some embodiments, each instance of R^{1C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{1C} is independently —S(O)₂R, —S(O)₂NR₂, or —S(O)₂F. In some embodiments, each instance of R^{1C} is independently —S(O)R, —S(O)NR₂, or —S(O)(NR)R. In some embodiments, each instance of R^{1C} is independently —SR, —S(O)₂R, or —S(O)R. In some embodiments, each instance of R^{1C} is independently —S(O)₂NR₂, —S(O)NR₂, or —S(O)(NR)R. In some embodiments, each instance of R^{1C} is independently —S(O)₂NR₂ or —S(O)NR₂. In some embodiments, each instance of R^{1C} is independently —SR, —S(O)₂R, —S(O)₂NR₂, or —S(O)R.

In some embodiments, each instance of R^{1C} is independently —N(R)C(O)OR, —N(R)C(O)R, or —N(R)C(O)NR₂. In some embodiments, each instance of R^{1C} is independently —N(R)S(O)₂NR₂ or —N(R)S(O)₂R. In some embodiments, each instance of R^{1C} is independently —N(R)C(O)OR or —N(R)C(O)R. In some embodiments, each instance of R^{1C} is independently —N(R)C(O)NR₂ or —N(R)S(O)₂NR₂. In some embodiments, each instance of R^{1C} is independently —N(R)C(O)OR, —N(R)C(O)R, or —N(R)S(O)₂R.

In some embodiments, each instance of R^{1C} is independently —NR₂, —N(R)C(O)OR, —N(R)C(O)R, or —N(R)C(O)NR₂. In some embodiments, each instance of R^{1C} is independently —NR₂, —N(R)C(O)OR, or —N(R)C(O)R. In some embodiments, each instance of R^{1C} is independently —NR₂, —N(R)C(O)OR, —N(R)C(O)R, or —N(R)S(O)₂R.

In some embodiments, each instance of R^{1C} is independently an optionally substituted C₁₋₆ aliphatic. In some embodiments, each instance of R^{1C} is independently an optionally substituted phenyl. In some embodiments, each instance of R^{1C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{1C} is independently an optionally substituted 5-6 membered monocyclic heteroaryl ring having 1-4 heteroatoms independently selected from nitrogen, oxygen, and sulfur.

In some embodiments, each instance of R^{1C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{1C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{1C} is independently an optionally substituted C₁₋₆ aliphatic or an optionally substituted phenyl. In some embodiments, each instance of R^{1C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{1C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{1C} is independently a C₁₋₆ aliphatic. In some embodiments, R^{1C} is phenyl. In some embodiments, each instance of R^{1C} is independently a 3-7 membered saturated or partially unsaturated monocyclic heterocyclic ring having 1-2 heteroatoms independently selected from nitrogen, oxygen, and sulfur. In some embodiments, each instance of R^{1C} is independently a 5-6 membered monocyclic heteroaryl ring having 1-4 heteroatoms independently selected from nitrogen, oxygen, and sulfur.

In some embodiments, each instance of R^{1C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{1C} is independently phenyl or a 5-6 membered monocyclic heteroaryl ring having 1-4 heteroatoms independently selected from nitrogen, oxygen, and sulfur.

In some embodiments, each instance of R^{1C} is independently a C₁₋₆ aliphatic or phenyl. In some embodiments, each instance of R^{1C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{1C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{1C} is independently halogen, —CN, —O-(optionally substituted C₁₋₆ aliphatic), or an optionally substituted C₁₋₆ aliphatic. In some embodiments, each instance of R^{1C} is independently halogen, —CN, —O—(C₁₋₆ aliphatic), or C₁₋₆ aliphatic; wherein each C₁₋₆ aliphatic is optionally substituted with one or more halogen atoms. In some embodiments, each instance of R^{1C} is independently halogen or C₁₋₃ aliphatic optionally substituted with 1-3 halogen. In some embodiments, each instance of R^{1C} is independently fluorine, chlorine, —CH₃, —CHF₂, or —CF₃.

In some embodiments, each instance of R^{1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{1C} is independently selected from the groups depicted in the compounds in Table 1.

As defined generally above, each instance of R^{2C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{2C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{2C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{2C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{2C} is oxo. In some embodiments, R^{2C} is deuterium. In some embodiments, each instance of R^{2C} is independently halogen. In some embodiments, R^{2C} is —CN. In some embodiments, R^{2C} is —NO₂. In some embodiments, R^{2C} is —OR. In some embodiments, R^{2C} is —SR. In some embodiments, R^{2C} is —NR₂. In some embodiments, R^{2C} is —S(O)₂R. In some embodiments, R^{2C} is —S(O)₂NR₂. In some embodiments, R^{2C} is —S(O)₂F. In some embodiments, R^{2C} is —S(O)R. In some embodiments, R^{2C} is —S(O)NR₂. In some embodiments, R^{2C} is —S(O)(NR)R. In some embodiments, R^{2C} is —C(O)R. In some embodiments, R^{2C} is —C(O)OR. In some embodiments, R^{2C} is —C(O)NR₂. In some embodiments, R^{2C} is —C(O)N(R)OR. In some embodiments, R^{2C} is —OC(O)R. In some embodiments, R^{2C} is —OC(O)NR₂. In some embodiments, R^{2C} is —N(R)C(O)OR. In some embodiments, R^{2C} is —N(R)C(O)R. In some embodiments, R^{2C} is —N(R)C(O)NR₂. In some embodiments, R^{2C} is —N(R)C(NR)NR₂. In some embodiments, R^{2C} is —N(R)S(O)₂NR₂. In some embodiments, R^{2C} is —N(R)S(O)₂R. In some embodiments, R^{2C} is —P(O)R₂. In some embodiments, R^{2C} is —P(O)(R)OR. In some embodiments, R^{2C} is —B(OR)₂.

In some embodiments, each instance of R^{2C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{2C} is independently halogen, —CN, or —NO₂. In some embodiments, each instance of R^{2C} is independently —OR, —SR, or —NR₂. In some embodiments, each instance of R^{2C} is independently —S(O)₂R, —S(O)₂NR₂, —S(O)₂F, —S(O)R, —S(O)NR₂, or —S(O)(NR)R. In some embodiments, each instance of R^{2C} is independently —C(O)R, —C(O)OR, —C(O)NR₂, or —C(O)N(R)OR. In some embodiments, each instance of R^{2C} is independently —OC(O)R or —OC(O)NR₂. In some embodiments, each instance of R^{2C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{2C} is independently —P(O)R₂ or —P(O)(R)OR.

In some embodiments, each instance of R^{2C} is independently —OR, —OC(O)R, or —OC(O)NR₂. In some embodiments, each instance of R^{2C} is independently —SR, —S(O)₂R, —S(O)₂NR₂, —S(O)₂F, —S(O)R, —S(O)NR₂, or —S(O)(NR)R. In some embodiments, each instance of R^{2C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{2C} is independently —S(O)₂R, —S(O)₂NR₂, or —S(O)₂F. In some embodiments, each instance of R^{2C} is independently —S(O)R, —S(O)NR₂, or —S(O)(NR)R. In some embodiments, each instance of R^{2C} is independently —SR, —S(O)₂R, or —S(O)R. In some embodiments, each instance of R^{2C} is independently —S(O)₂NR₂, —S(O)NR₂, or —S(O)(NR)R. In some embodiments, each instance of R^{2C} is independently —S(O)₂NR₂ or —S(O)NR₂. In some embodiments, each instance of R^{2C} is independently —SR, —S(O)₂R, —S(O)₂NR₂, or —S(O)R.

In some embodiments, each instance of R^{2C} is independently —N(R)C(O)OR, —N(R)C(O)R, or —N(R)C(O)NR₂. In some embodiments, each instance of R^{2C} is independently —N(R)S(O)₂NR₂ or —N(R)S(O)₂R. In some embodiments, each instance of R^{2C} is independently —N(R)C(O)OR or —N(R)C(O)R. In some embodiments, each instance of R^{2C} is independently —N(R)C(O)NR₂ or —N(R)S(O)₂NR₂. In some embodiments, each instance of R^{2C} is independently —N(R)C(O)OR, —N(R)C(O)R, or —N(R)S(O)₂R.

In some embodiments, each instance of R^{2C} is independently —NR₂, —N(R)C(O)OR, —N(R)C(O)R, or —N(R)C(O)NR₂. In some embodiments, each instance of R^{2C} is independently —NR₂, —N(R)C(O)OR, or —N(R)C(O)R. In some embodiments, each instance of R^{2C} is independently —NR₂, —N(R)C(O)OR, —N(R)C(O)R, or —N(R)S(O)₂R.

In some embodiments, each instance of R^{2C} is independently an optionally substituted C₁₋₆ aliphatic. In some embodiments, each instance of R^{2C} is independently an optionally substituted phenyl. In some embodiments, each instance of R^{2C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{2C} is independently an optionally substituted 5-6 membered monocyclic heteroaryl ring having 1-4 heteroatoms independently selected from nitrogen, oxygen, and sulfur.

In some embodiments, each instance of R^{2C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{2C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{2C} is independently an optionally substituted C₁₋₆ aliphatic or an optionally substituted phenyl. In some embodiments, each instance of R^{2C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{2C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{2C} is independently a C₁₋₆ aliphatic. In some embodiments, R^{2C} is phenyl. In some embodiments, each instance of R^{2C} is independently a 3-7 membered saturated or partially unsaturated monocyclic heterocyclic ring having 1-2 heteroatoms independently selected from nitrogen, oxygen, and sulfur. In some embodiments, each instance of R^{2C} is independently a 5-6 membered monocyclic heteroaryl ring having 1-4 heteroatoms independently selected from nitrogen, oxygen, and sulfur.

In some embodiments, each instance of R^{2C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{2C} is independently phenyl or a 5-6 membered monocyclic heteroaryl ring having 1-4 heteroatoms independently selected from nitrogen, oxygen, and sulfur.

In some embodiments, each instance of R^{2C} is independently a C₁₋₆ aliphatic or phenyl. In some embodiments, each instance of R^{2C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{2C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{2C} is independently halogen, —CN, —O-(optionally substituted C₁₋₆ aliphatic), or an optionally substituted C₁₋₆ aliphatic. In some embodiments, each instance of R^{2C} is independently halogen, —CN, —O—(C₁₋₆ aliphatic), or C₁₋₆ aliphatic; wherein each C₁₋₆ aliphatic is optionally substituted with one or more halogen atoms. In some embodiments, each instance of R^{2C} is independently halogen or C₁₋₃ aliphatic optionally substituted with 1-3 halogen. In some embodiments, each instance of R^{2C} is independently fluorine, chlorine, —CH₃, —CHF₂, or —CF₃.

In some embodiments, each instance of R^{2C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{2C} is independently a C₁₋₆ aliphatic optionally substituted with (i) 1 or 2 groups independently selected from —O—(C₁₋₆ aliphatic), —OH, —N(C₁₋₆ aliphatic)₂, and —CN, and (ii) 1, 2, or 3 atoms independently selected from halogen and deuterium. In some embodiments, each instance of R^{2C} is independently a C₁₋₆ aliphatic optionally substituted with (i) 1 or 2 groups independently selected from —O—(C₁₋₆ aliphatic), —OH, —N(C₁₋₆ aliphatic)₂, and —CN, and (ii) 1, 2, or 3 halogen atoms. In some embodiments, each instance of R^{2C} is independently a C₁₋₆ aliphatic optionally substituted with 1 or 2 groups independently selected from —O—(C₁₋₆ aliphatic), —OH, —N(C₁₋₆ aliphatic)₂, and —CN. In some embodiments, each instance of R^{2C} is independently a C₁₋₆ aliphatic optionally substituted with 1, 2, or 3 atoms independently selected from halogen and deuterium. In some embodiments, each instance of R^{2C} is independently a C₁₋₆ aliphatic optionally substituted with 1, 2, or 3 atoms independently selected from halogen.

In some embodiments, each instance of R^{2C} is independently oxo, deuterium, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{2C} is independently oxo, deuterium, halogen, or —CN. In some embodiments, each instance of R^{2C} is independently oxo, —OH, —O—(C₁₋₃ aliphatic), or C₁₋₃ aliphatic, wherein each C₁₋₃ aliphatic is optionally substituted with one or more halogen atoms. In some embodiments, each instance of R^{2C} is independently —O—(C₁₋₃ aliphatic) or C₁₋₃ aliphatic, wherein each C₁₋₃ aliphatic is optionally substituted with one or more halogen atoms. In some embodiments, each instance of R^{2C} is independently —O—(C₁₋₃ aliphatic) or C₁₋₃ aliphatic.

In some embodiments, each instance of R^{2C} is independently selected from the groups depicted in the compounds in Table 1.

As defined generally above, each instance of R^{TC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{TC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{TC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{TC} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{TC} is oxo. In some embodiments, R^{TC} is deuterium. In some embodiments, each instance of R^{TC} is independently halogen. In some embodiments, R^{TC} is —CN. In some embodiments, R^{TC} is —NO₂. In some embodiments, R^{TC} is —OR. In some embodiments, R^{TC} is —SR. In some embodiments, R^{TC} is —NR₂. In some embodiments, R^{TC} is —S(O)₂R. In some embodiments, R^{TC} is —S(O)₂NR₂. In some embodiments, R^{TC} is —S(O)₂F. In some embodiments, R^{TC} is —S(O)R. In some embodiments, R^{TC} is —S(O)NR₂. In some embodiments, R^{TC} is —S(O)(NR)R. In some embodiments, R^{TC} is —C(O)R. In some embodiments, R^{TC} is —C(O)OR. In some embodiments, R^{TC} is —C(O)NR₂. In some embodiments, R^{TC} is —C(O)N(R)OR. In some embodiments, R^{TC} is —OC(O)R. In some embodiments, R^{TC} is —OC(O)NR₂. In some embodiments, R^{TC} is —N(R)C(O)OR. In some embodiments, R^{TC} is —N(R)C(O)R. In some embodiments, R^{TC} is —N(R)C(O)NR₂. In some embodiments, R^{TC} is —N(R)C(NR)NR₂. In some embodiments, R^{TC} is —N(R)S(O)₂NR₂. In some embodiments, R^{TC} is —N(R)S(O)₂R. In some embodiments, R^{TC} is —P(O)R₂. In some embodiments, R^{TC} is —P(O)(R)OR. In some embodiments, R^{TC} is —B(OR)₂.

In some embodiments, each instance of R^{TC} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{TC} is independently halogen, —CN, or —NO₂. In some embodiments, each instance of R^{TC} is independently —OR, —SR, or —NR₂. In some embodiments, each instance of R^{TC} is independently —S(O)₂R, —S(O)₂NR₂, —S(O)₂F, —S(O)R, —S(O)NR₂, or —S(O)(NR)R. In some embodiments, each instance of R^{TC} is independently —C(O)R, —C(O)OR, —C(O)NR₂, or —C(O)N(R)OR. In some embodiments, each instance of R^{TC} is independently —OC(O)R or —OC(O)NR₂. In some embodiments, each instance of R^{TC} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{TC} is independently —P(O)R₂ or —P(O)(R)OR.

In some embodiments, each instance of R^{TC} is independently —OR, —OC(O)R, or —OC(O)NR₂. In some embodiments, each instance of R^{TC} is independently —SR, —S(O)₂R, —S(O)₂NR₂, —S(O)₂F, —S(O)R, —S(O)NR₂, or —S(O)(NR)R. In some embodiments, each instance of R^{TC} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{TC} is independently —S(O)₂R, —S(O)₂NR₂, or —S(O)₂F. In some embodiments, each instance of R^{TC} is independently —S(O)R, —S(O)NR₂, or —S(O)(NR)R. In some embodiments, each instance of R^{TC} is independently —SR, —S(O)₂R, or —S(O)R. In some embodiments, each instance of R^{TC} is independently —S(O)₂NR₂, —S(O)NR₂, or —S(O)(NR)R. In some embodiments, each instance of R^{TC} is independently —S(O)₂NR₂ or —S(O)NR₂. In some embodiments, each instance of R^{TC} is independently —SR, —S(O)₂R, —S(O)₂NR₂, or —S(O)R.

In some embodiments, each instance of R^{TC} is independently —N(R)C(O)OR, —N(R)C(O)R, or —N(R)C(O)NR₂. In some embodiments, each instance of R^{TC} is independently —N(R)S(O)₂NR₂ or —N(R)S(O)₂R. In some embodiments, each instance of R^{TC} is independently —N(R)C(O)OR or —N(R)C(O)R. In some embodiments, each instance of R^{TC} is independently —N(R)C(O)NR₂ or —N(R)S(O)₂NR₂. In some embodiments, each instance of R^{TC} is independently —N(R)C(O)OR, —N(R)C(O)R, or —N(R)S(O)₂R.

In some embodiments, each instance of R^{TC} is independently —NR₂, —N(R)C(O)OR, —N(R)C(O)R, or —N(R)C(O)NR₂. In some embodiments, each instance of R^{TC} is independently —NR₂, —N(R)C(O)OR, or —N(R)C(O)R. In some embodiments, each instance of R^{TC} is independently —NR₂, —N(R)C(O)OR, —N(R)C(O)R, or —N(R)S(O)₂R.

In some embodiments, each instance of R^{TC} is independently an optionally substituted C₁₋₆ aliphatic. In some embodiments, each instance of R^{TC} is independently an optionally substituted phenyl. In some embodiments, each instance of R^{TC} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{TC} is independently an optionally substituted 5-6 membered monocyclic heteroaryl ring having 1-4 heteroatoms independently selected from nitrogen, oxygen, and sulfur.

In some embodiments, each instance of R^{TC} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{TC} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{TC} is independently an optionally substituted C₁₋₆ aliphatic or an optionally substituted phenyl. In some embodiments, each instance of R^{TC} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{TC} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{TC} is independently a C₁₋₆ aliphatic. In some embodiments, R^{TC} is phenyl. In some embodiments, each instance of R^{TC} is independently a 3-7 membered saturated or partially unsaturated monocyclic heterocyclic ring having 1-2 heteroatoms independently selected from nitrogen, oxygen, and sulfur. In some embodiments, each instance of R^{TC} is independently a 5-6 membered monocyclic heteroaryl ring having 1-4 heteroatoms independently selected from nitrogen, oxygen, and sulfur.

In some embodiments, each instance of R^{TC} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{TC} is independently phenyl or a 5-6 membered monocyclic heteroaryl ring having 1-4 heteroatoms independently selected from nitrogen, oxygen, and sulfur.

In some embodiments, each instance of R^{TC} is independently a C₁₋₆ aliphatic or phenyl. In some embodiments, each instance of R^{TC} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{TC} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{TC} is independently halogen, —CN, —O-(optionally substituted C₁₋₆ aliphatic), or an optionally substituted C₁₋₆ aliphatic. In some embodiments, each instance of R^{TC} is independently halogen, —CN, —O—(C₁₋₆ aliphatic), or C₁₋₆ aliphatic; wherein each C₁₋₆ aliphatic is optionally substituted with one or more halogen atoms. In some embodiments, each instance of R^{TC} is independently halogen or C₁₋₃ aliphatic optionally substituted with 1-3 halogen. In some embodiments, each instance of R^{TC} is independently fluorine, chlorine, —CH₃, —CHF₂, or —CF₃.

In some embodiments, each instance of R^{TC} is a C₁₋₆ aliphatic optionally substituted with (i) 1 or 2 groups independently selected from —O—(C₁₋₆ aliphatic), —OH, —N(C₁₋₆ aliphatic)₂, and —CN, and (ii) 1, 2, or 3 atoms independently selected from halogen and deuterium. In some embodiments, each instance of R^{TC} is a C₁₋₆ aliphatic optionally substituted with (i) 1 or 2 groups independently selected from —O—(C₁₋₆ aliphatic), —OH, —N(C₁₋₆ aliphatic)₂, and —CN, and (ii) 1, 2, or 3 halogen atoms. In some embodiments, each instance of R^{TC} is a C₁₋₆ aliphatic optionally substituted with 1 or 2 groups independently selected from —O—(C₁₋₆ aliphatic), —OH, —N(C₁₋₆ aliphatic)₂, and —CN. In some embodiments, each instance of R^{TC} is a C₁₋₆ aliphatic optionally substituted with 1, 2, or 3 atoms independently selected from halogen and deuterium. In some embodiments, each instance of R^{TC} is a C₁₋₆ aliphatic optionally substituted with 1, 2, or 3 atoms independently selected from halogen.

In some embodiments, each instance of R^{TC} is independently oxo, deuterium, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{TC} is independently oxo, deuterium, halogen, or —CN. In some embodiments, each instance of R^{TC} is independently oxo, —OH, —O—(C₁₋₃ aliphatic), or C₁₋₃ aliphatic, wherein each C₁₋₃ aliphatic is optionally substituted with one or more halogen atoms. In some embodiments, each instance of R^{TC} is independently —O—(C₁₋₃ aliphatic) or C₁₋₃ aliphatic, wherein each C₁₋₃ aliphatic is optionally substituted with one or more halogen atoms. In some embodiments, each instance of R^{TC} is independently —O—(C₁₋₃ aliphatic) or C₁₋₃ aliphatic.

In some embodiments, each instance of R^{TC} is independently halogen, —CN, —OH, —O-(optionally substituted C₁₋₃ aliphatic), or an optionally substituted C₁₋₃ aliphatic. In some embodiments, each instance of R^{TC} is independently halogen, —OH, —O—(C₁₋₃ aliphatic), or C₁₋₃ aliphatic, wherein each C₁₋₃ aliphatic is optionally substituted with 1-3 halogen. In some embodiments, each instance of R^{TC} is independently fluorine, chlorine, —OH, —OCH₃, —OCF₃, —CH₃, —CHF₂, or —CF₃. In some embodiments, each instance of R^{TC} is independently fluorine or —OH.

In some embodiments, each instance of R^{TC} is independently oxo, deuterium, halogen, —CN, —OH, —O-(optionally substituted C₁₋₃ aliphatic), or an optionally substituted C₁₋₃ aliphatic. In some embodiments, each instance of R^{TC} is independently oxo, deuterium, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{TC} is independently oxo, deuterium, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{TC} is independently oxo, deuterium, fluorine, chlorine, —CN, —OH, —OCH₃, —OCF₃, —CH₃, —CHF₂, or —CF₃. In some embodiments, each instance of R^{TC} is independently oxo, deuterium, —CN, fluorine, or —OH. In some embodiments, each instance of R^{TC} is independently oxo, deuterium, —CN, —CH₃, or —CHF₂. In some embodiments, each instance of R^{TC} is independently deuterium, —CN, —CH₃, or —CHF₂.

In some embodiments, each instance of R^{TC} is independently oxo, halogen, —CN, —OH, —O-(optionally substituted C₁₋₃ aliphatic), or an optionally substituted C-3 aliphatic. In some embodiments, each instance of R^{TC} is independently oxo, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{TC} is independently oxo, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{TC} is independently oxo, fluorine, chlorine, —CN, —OH, —OCH₃, —OCF₃, —CH₃, —CHF₂, or —CF₃. In some embodiments, each instance of R^{TC} is independently oxo, —CN, fluorine, or —OH. In some embodiments, each instance of R^{TC} is independently oxo, —CN, —CH₃, or —CHF₂. In some embodiments, each instance of R^{TC} is independently —CN, —CH₃, or —CHF₂.

In some embodiments, each instance of R^{TC} is independently selected from the groups depicted in the compounds in Table 1.

As defined generally above, each instance of R^{TTC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{TTC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R², —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{TTC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{TTC} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{TTC} is oxo. In some embodiments, R^{TTC} is deuterium. In some embodiments, each instance of R^{TTC} is independently halogen. In some embodiments, R^{TTC} is —CN. In some embodiments, R^{TTC} is —NO₂. In some embodiments, R^{TTC} is —OR. In some embodiments, R^{TTC} is —SR. In some embodiments, R^{TTC} is —NR₂. In some embodiments, R^{TTC} is —S(O)₂R. In some embodiments, R^{TTC} is —S(O)₂NR₂. In some embodiments, R^{TTC} is —S(O)₂F. In some embodiments, R^{TTC} is —S(O)R. In some embodiments, R^{TTC} is —S(O)NR₂. In some embodiments, R^{TTC} is —S(O)(NR)R. In some embodiments, R^{TTC} is —C(O)R. In some embodiments, R^{TTC} is —C(O)OR. In some embodiments, R^{TTC} is —C(O)NR₂. In some embodiments, R^{TTC} is —C(O)N(R)OR. In some embodiments, R^{TTC} is —OC(O)R. In some embodiments, R^{TTC} is —OC(O)NR₂. In some embodiments, R^{TTC} is —N(R)C(O)OR. In some embodiments, R^{TTC} is —N(R)C(O)R. In some embodiments, R^{TTC} is —N(R)C(O)NR₂. Tn some embodiments, R^{TTC} is —N(R)C(NR)NR₂. In some embodiments, R^{TTC} is —N(R)S(O)₂NR₂. In some embodiments, R^{TTC} is —N(R)S(O)₂R. In some embodiments, R^{TTC} is —P(O)R₂. In some embodiments, R^{TTC} is —P(O)(R)OR. In some embodiments, R^{TTC} is —B(OR)₂.

In some embodiments, each instance of R^{TTC} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{TTC} is independently halogen, —CN, or —NO₂. In some embodiments, each instance of R^{TTC} is independently —OR, —SR, or —NR₂. In some embodiments, each instance of R^{TTC} is independently —S(O)₂R, —S(O)₂NR₂, —S(O)₂F, —S(O)R, —S(O)NR₂, or —S(O)(NR)R. In some embodiments, each instance of R^{TTC} is independently —C(O)R, —C(O)OR, —C(O)NR₂, or —C(O)N(R)OR. In some embodiments, each instance of R^{TTC} is independently —OC(O)R or —OC(O)NR₂. In some embodiments, each instance of R^{TTC} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{TTC} is independently —P(O)R₂ or —P(O)(R)OR.

In some embodiments, each instance of R^{TTC} is independently —OR, —OC(O)R, or —OC(O)NR₂. In some embodiments, each instance of R^{TTC} is independently —SR, —S(O)₂R, —S(O)₂NR₂, —S(O)₂F, —S(O)R, —S(O)NR₂, or —S(O)(NR)R. In some embodiments, each instance of R^{TTC} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{TTC} is independently —S(O)₂R, —S(O)₂NR₂, or —S(O)₂F. In some embodiments, each instance of R^{TTC} is independently —S(O)R, —S(O)NR₂, or —S(O)(NR)R. In some embodiments, each instance of R^{TTC} is independently —SR, —S(O)₂R, or —S(O)R. In some embodiments, each instance of R^{TTC} is independently —S(O)₂NR₂, —S(O)NR₂, or —S(O)(NR)R. In some embodiments, each instance of R^{TTC} is independently —S(O)₂NR₂ or —S(O)NR₂. In some embodiments, each instance of R^{TTC} is independently —SR, —S(O)₂R, —S(O)₂NR₂, or —S(O)R.

In some embodiments, each instance of R^{TTC} is independently —N(R)C(O)OR, —N(R)C(O)R, or —N(R)C(O)NR₂. In some embodiments, each instance of R^{TTC} is independently —N(R)S(O)₂NR₂ or —N(R)S(O)₂R. In some embodiments, each instance of R^{TTC} is independently —N(R)C(O)OR or —N(R)C(O)R. In some embodiments, each instance of R^{TTC} is independently —N(R)C(O)NR₂ or —N(R)S(O)₂NR₂. In some embodiments, each instance of R^{TTC} is independently —N(R)C(O)OR, —N(R)C(O)R, or —N(R)S(O)₂R.

In some embodiments, each instance of R^{TTC} is independently —NR₂, —N(R)C(O)OR, —N(R)C(O)R, or —N(R)C(O)NR₂. In some embodiments, each instance of R^{TTC} is independently —NR₂, —N(R)C(O)OR, or —N(R)C(O)R. In some embodiments, each instance of R^{TTC} is independently —NR₂, —N(R)C(O)OR, —N(R)C(O)R, or —N(R)S(O)₂R.

In some embodiments, each instance of R^{TTC} is independently an optionally substituted C₁₋₆ aliphatic. In some embodiments, each instance of R^{TTC} is independently an optionally substituted phenyl. In some embodiments, each instance of R^{TTC} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{TTC} is independently an optionally substituted 5-6 membered monocyclic heteroaryl ring having 1-4 heteroatoms independently selected from nitrogen, oxygen, and sulfur.

In some embodiments, each instance of R^{TTC} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{TTC} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{TTC} is independently an optionally substituted C₁₋₆ aliphatic or an optionally substituted phenyl. In some embodiments, each instance of R^{TTC} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{TTC} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{TTC} is independently a C₁₋₆ aliphatic. In some embodiments, R^{TTC} is phenyl. In some embodiments, each instance of R^{TTC} is independently a 3-7 membered saturated or partially unsaturated monocyclic heterocyclic ring having 1-2 heteroatoms independently selected from nitrogen, oxygen, and sulfur. In some embodiments, each instance of R^{TTC} is independently a 5-6 membered monocyclic heteroaryl ring having 1-4 heteroatoms independently selected from nitrogen, oxygen, and sulfur.

In some embodiments, each instance of R^{TTC} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{TTC} is independently phenyl or a 5-6 membered monocyclic heteroaryl ring having 1-4 heteroatoms independently selected from nitrogen, oxygen, and sulfur.

In some embodiments, each instance of R^{TTC} is independently a C₁₋₆ aliphatic or phenyl. In some embodiments, each instance of R^{TTC} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{TTC} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{TTC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{TTC} is independently halogen, —CN, —OH, —O-(optionally substituted C₁₋₃ aliphatic), or an optionally substituted C₁₋₃ aliphatic. In some embodiments, each instance of R^{TTC} is independently halogen, —OH, —O—(C₁₋₃ aliphatic), or C₁₋₃ aliphatic, wherein each C₁₋₃ aliphatic is optionally substituted with 1-3 halogen. In some embodiments, each instance of R^{TTC} is independently fluorine, chlorine, —OH, —OCH₃, —OCF₃, —CH₃, —CHF₂, or —CF₃. In some embodiments, each instance of R^{TTC} is independently fluorine or —OH.

In some embodiments, each instance of R^{TTC} is independently oxo, deuterium, halogen, —CN, —OH, —O-(optionally substituted C₁₋₃ aliphatic), or an optionally substituted C₁₋₃ aliphatic. In some embodiments, each instance of R^{TTC} is independently oxo, deuterium, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{TTC} is independently oxo, deuterium, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{TTC} is independently oxo, deuterium, fluorine, chlorine, —CN, —OH, —OCH₃, —OCF₃, —CH₃, —CHF₂, or —CF₃. In some embodiments, each instance of R^{TTC} is independently oxo, deuterium, —CN, fluorine, or —OH. In some embodiments, each instance of R^{TTC} is independently oxo, deuterium, —CN, —CH₃, or —CHF₂. In some embodiments, each instance of R^{TTC} is independently deuterium, —CN, —CH₃, or —CHF₂.

In some embodiments, each instance of R^{TTC} is independently oxo, halogen, —CN, —OH, —O-(optionally substituted C₁₋₃ aliphatic), or an optionally substituted C₁₋₃ aliphatic. In some embodiments, each instance of R^{TTC} is independently oxo, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{TTC} is independently oxo, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{TTC} is independently oxo, fluorine, chlorine, —CN, —OH, —OCH₃, —OCF₃, —CH₃, —CHF₂, or —CF₃. In some embodiments, each instance of R^{TTC} is independently oxo, —CN, fluorine, or —OH. In some embodiments, each instance of R^{TTC} is independently oxo, —CN, —CH₃, or —CHF₂. In some embodiments, each instance of R^{TTC} is independently —CN, —CH₃, or —CHF₂.

In some embodiments, each instance of R^{TTC} is independently selected from the groups depicted in the compounds in Table 1.

As defined generally above, each instance of R^{1C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R², —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{11C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{11C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{11C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{11C} is oxo. In some embodiments, R^{11C} is deuterium. In some embodiments, each instance of R^{11C} is independently halogen. In some embodiments, R^{11C} is —CN. In some embodiments, R^{11C} is —NO₂. In some embodiments, R^{11C} is —OR. In some embodiments, R^{11C} is —SR. In some embodiments, R^{11C} is —NR₂. In some embodiments, R^{11C} is —S(O)₂R. In some embodiments, R^{11C} is —S(O)₂NR₂. In some embodiments, R^{11C} is —S(O)₂F. In some embodiments, R^{11C} is —S(O)R. In some embodiments, R^{11C} is —S(O)NR₂. In some embodiments, R^{11C} is —S(O)(NR)R. In some embodiments, R^{11C} is —C(O)R. In some embodiments, R^{11C} is —C(O)OR. In some embodiments, R^{11C} is —C(O)NR₂. In some embodiments, R^{11C} is —C(O)N(R)OR. In some embodiments, R^{11C} is —OC(O)R. In some embodiments, R^{11C} is —OC(O)NR₂. In some embodiments, R^{11C} is —N(R)C(O)OR. In some embodiments, R^{11C} is —N(R)C(O)R. In some embodiments, R^{11C} is —N(R)C(O)NR₂. In some embodiments, R^{11C} is —N(R)C(NR)NR₂. In some embodiments, R^{11C} is —N(R)S(O)₂NR₂. In some embodiments, R^{11C} is —N(R)S(O)₂R. In some embodiments, R^{11C} is —P(O)R₂. In some embodiments, R^{11C} is —P(O)(R)OR. In some embodiments, R^{11C} is —B(OR)₂.

In some embodiments, each instance of R^{11C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{11C} is independently halogen, —CN, or —NO₂. In some embodiments, each instance of R^{11C} is independently —OR, —SR, or —NR₂. In some embodiments, each instance of R^{11C} is independently —S(O)₂R, —S(O)₂NR₂, —S(O)₂F, —S(O)R, —S(O)NR₂, or —S(O)(NR)R. In some embodiments, each instance of R^{11C} is independently —C(O)R, —C(O)OR, —C(O)NR₂, or —C(O)N(R)OR. In some embodiments, each instance of R^{11C} is independently —OC(O)R or —OC(O)NR₂. In some embodiments, each instance of R^{11C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{11C} is independently —P(O)R₂ or —P(O)(R)OR.

In some embodiments, each instance of R^{11C} is independently —OR, —OC(O)R, or —OC(O)NR₂. In some embodiments, each instance of R^{11C} is independently —SR, —S(O)₂R, —S(O)₂NR₂, —S(O)₂F, —S(O)R, —S(O)NR₂, or —S(O)(NR)R. In some embodiments, each instance of R^{11C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{11C} is independently —S(O)₂R, —S(O)₂NR₂, or —S(O)₂F. In some embodiments, each instance of R^{11C} is independently —S(O)R, —S(O)NR₂, or —S(O)(NR)R. In some embodiments, each instance of R^{11C} is independently —SR, —S(O)₂R, or —S(O)R. In some embodiments, each instance of R^{11C} is independently —S(O)₂NR₂, —S(O)NR₂, or —S(O)(NR)R. In some embodiments, each instance of R^{11C} is independently —S(O)₂NR₂ or —S(O)NR₂. In some embodiments, each instance of R^{11C} is independently —SR, —S(O)₂R, —S(O)₂NR₂, or —S(O)R.

In some embodiments, each instance of R^{11C} is independently —N(R)C(O)OR, —N(R)C(O)R, or —N(R)C(O)NR₂. In some embodiments, each instance of R^{11C} is independently —N(R)S(O)₂NR₂ or —N(R)S(O)₂R. In some embodiments, each instance of R^{11C} is independently —N(R)C(O)OR or —N(R)C(O)R. In some embodiments, each instance of R^{11C} is independently —N(R)C(O)NR₂ or —N(R)S(O)₂NR₂. In some embodiments, each instance of R^{11C} is independently —N(R)C(O)OR, —N(R)C(O)R, or —N(R)S(O)₂R.

In some embodiments, each instance of R^{11C} is independently —NR₂, —N(R)C(O)OR, —N(R)C(O)R, or —N(R)C(O)NR₂. In some embodiments, each instance of R^{11C} is independently —NR₂, —N(R)C(O)OR, or —N(R)C(O)R. In some embodiments, each instance of R^{11C} is independently —NR₂, —N(R)C(O)OR, —N(R)C(O)R, or —N(R)S(O)₂R.

In some embodiments, each instance of R^{11C} is independently an optionally substituted C₁₋₆ aliphatic. In some embodiments, each instance of R^{11C} is independently an optionally substituted phenyl. In some embodiments, each instance of R^{11C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{11C} is independently an optionally substituted 5-6 membered monocyclic heteroaryl ring having 1-4 heteroatoms independently selected from nitrogen, oxygen, and sulfur.

In some embodiments, each instance of R^{11C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{11C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{11C} is independently an optionally substituted C₁₋₆ aliphatic or an optionally substituted phenyl. In some embodiments, each instance of R^{11C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{11C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{11C} is independently a C₁₋₆ aliphatic. In some embodiments, R^{11C} is phenyl. In some embodiments, each instance of R^{11C} is independently a 3-7 membered saturated or partially unsaturated monocyclic heterocyclic ring having 1-2 heteroatoms independently selected from nitrogen, oxygen, and sulfur. In some embodiments, each instance of R^{11C} is independently a 5-6 membered monocyclic heteroaryl ring having 1-4 heteroatoms independently selected from nitrogen, oxygen, and sulfur.

In some embodiments, each instance of R^{11C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{11C} is independently phenyl or a 5-6 membered monocyclic heteroaryl ring having 1-4 heteroatoms independently selected from nitrogen, oxygen, and sulfur.

In some embodiments, each instance of R^{11C} is independently a C₁₋₆ aliphatic or phenyl. In some embodiments, each instance of R^{11C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{11C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{11C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{11C} is independently halogen, —CN, —OH, —O-(optionally substituted C₁₋₃ aliphatic), or an optionally substituted C₁₋₃ aliphatic. In some embodiments, each instance of R^{11C} is independently halogen, —OH, —O—(C₁₋₃ aliphatic), or C₁₋₃ aliphatic, wherein each C₁₋₃ aliphatic is optionally substituted with 1-3 halogen. In some embodiments, each instance of R^{11C} is independently fluorine, chlorine, —OH, —OCH₃, —OCF₃, —CH₃, —CHF₂, or —CF₃. In some embodiments, each instance of R^{11C} is independently fluorine or —OH.

In some embodiments, each instance of R^{11C} is independently oxo, deuterium, halogen, —CN, —OH, —O-(optionally substituted C₁₋₃ aliphatic), or an optionally substituted C₁₋₃ aliphatic. In some embodiments, each instance of R^{11C} is independently oxo, deuterium, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{11C} is independently oxo, deuterium, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{11C} is independently oxo, deuterium, fluorine, chlorine, —CN, —OH, —OCH₃, —OCF₃, —CH₃, —CHF₂, or —CF₃. In some embodiments, each instance of R^{11C} is independently oxo, deuterium, —CN, fluorine, or —OH. In some embodiments, each instance of R^{11C} is independently oxo, deuterium, —CN, —CH₃, or —CHF₂. In some embodiments, each instance of R^{11C} is independently deuterium, —CN, —CH₃, or —CHF₂.

In some embodiments, each instance of R^{11C} is independently oxo, halogen, —CN, —OH, —O-(optionally substituted C₁₋₃ aliphatic), or an optionally substituted C₁₋₃ aliphatic. In some embodiments, each instance of R^{11C} is independently oxo, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{11C} is independently oxo, halogen, —CN, —OH, —O—(Ca-3 aliphatic), or C₁₋₃ aliphatic, wherein each C₁₋₃ aliphatic is optionally substituted with 1-3 halogen. In some embodiments, each instance of R^{11C} is independently oxo, fluorine, chlorine, —CN, —OH, —OCH₃, —OCF₃, —CH₃, —CHF₂, or —CF₃. In some embodiments, each instance of R^{11C} is independently oxo, —CN, fluorine, or —OH. In some embodiments, each instance of R^{11C} is independently oxo, —CN, —CH₃, or —CHF₂. In some embodiments, each instance of R^{11C} is independently —CN, —CH₃, or —CHF₂.

In some embodiments, each instance of R^{11C} is independently selected from the groups depicted in the compounds in Table 1.

As defined generally above, each instance of R^{22C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{22C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{22C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{22C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{22C} is oxo. In some embodiments, R^{22C} is deuterium. In some embodiments, each instance of R^{22C} is independently halogen. In some embodiments, R^{22C} is —CN. In some embodiments, R^{22C} is —NO₂. In some embodiments, R^{22C} is —OR. In some embodiments, R^{22C} is —SR. In some embodiments, R^{22C} is —NR₂. In some embodiments, R^{22C} is —S(O)₂R. In some embodiments, R^{22C} is —S(O)₂NR₂. In some embodiments, R^{22C} is —S(O)₂F. In some embodiments, R^{22C} is —S(O)R. In some embodiments, R^{22C} is —S(O)NR₂. In some embodiments, R^{22C} is —S(O)(NR)R. In some embodiments, R^{22C} is —C(O)R. In some embodiments, R^{22C} is —C(O)OR. In some embodiments, R^{22C} is —C(O)NR₂. In some embodiments, R^{22C} is —C(O)N(R)OR. In some embodiments, R^{22C} is —OC(O)R. In some embodiments, R^{22C} is —OC(O)NR₂. In some embodiments, R^{22C} is —N(R)C(O)OR. In some embodiments, R^{22C} is —N(R)C(O)R. In some embodiments, R^{22C} is —N(R)C(O)NR₂. In some embodiments, R^{22C} is —N(R)C(NR)NR₂. In some embodiments, R^{22C} is —N(R)S(O)₂NR₂. In some embodiments, R^{22C} is —N(R)S(O)₂R. In some embodiments, R^{22C} is —P(O)R₂. In some embodiments, R^{22C} is —P(O)(R)OR. In some embodiments, R^{22C} is —B(OR)₂.

In some embodiments, each instance of R^{22C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{22C} is independently halogen, —CN, or —NO₂. In some embodiments, each instance of R^{22C} is independently —OR, —SR, or —NR₂. In some embodiments, each instance of R^{22C} is independently —S(O)₂R, —S(O)₂NR₂, —S(O)₂F, —S(O)R, —S(O)NR₂, or —S(O)(NR)R. In some embodiments, each instance of R^{22C} is independently —C(O)R, —C(O)OR, —C(O)NR₂, or —C(O)N(R)OR. In some embodiments, each instance of R^{22C} is independently —OC(O)R or —OC(O)NR₂. In some embodiments, each instance of R^{22C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{22C} is independently —P(O)R₂ or —P(O)(R)OR.

In some embodiments, each instance of R^{22C} is independently —OR, —OC(O)R, or —OC(O)NR₂. In some embodiments, each instance of R^{22C} is independently —SR, —S(O)₂R, —S(O)₂NR₂, —S(O)₂F, —S(O)R, —S(O)NR₂, or —S(O)(NR)R. In some embodiments, each instance of R^{22C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{22C} is independently —S(O)₂R, —S(O)₂NR₂, or —S(O)₂F. In some embodiments, each instance of R^{22C} is independently —S(O)R, —S(O)NR₂, or —S(O)(NR)R. In some embodiments, each instance of R^{22C} is independently —SR, —S(O)₂R, or —S(O)R. In some embodiments, each instance of R^{22C} is independently —S(O)₂NR₂, —S(O)NR₂, or —S(O)(NR)R. In some embodiments, each instance of R^{22C} is independently —S(O)₂NR₂ or —S(O)NR₂. In some embodiments, each instance of R^{22C} is independently —SR, —S(O)₂R, —S(O)₂NR₂, or —S(O)R.

In some embodiments, each instance of R^{22C} is independently —N(R)C(O)OR, —N(R)C(O)R, or —N(R)C(O)NR₂. In some embodiments, each instance of R^{22C} is independently —N(R)S(O)₂NR₂ or —N(R)S(O)₂R. In some embodiments, each instance of R^{22C} is independently —N(R)C(O)OR or —N(R)C(O)R. In some embodiments, each instance of R^{22C} is independently —N(R)C(O)NR₂ or —N(R)S(O)₂NR₂. In some embodiments, each instance of R^{22C} is independently —N(R)C(O)OR, —N(R)C(O)R, or —N(R)S(O)₂R.

In some embodiments, each instance of R^{22C} is independently —NR₂, —N(R)C(O)OR, —N(R)C(O)R, or —N(R)C(O)NR₂. In some embodiments, each instance of R^{22C} is independently —NR₂, —N(R)C(O)OR, or —N(R)C(O)R. In some embodiments, each instance of R^{22C} is independently —NR₂, —N(R)C(O)OR, —N(R)C(O)R, or —N(R)S(O)₂R.

In some embodiments, each instance of R^{22C} is independently an optionally substituted C₁₋₆ aliphatic. In some embodiments, each instance of R^{22C} is independently an optionally substituted phenyl. In some embodiments, each instance of R^{22C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{22C} is independently an optionally substituted 5-6 membered monocyclic heteroaryl ring having 1-4 heteroatoms independently selected from nitrogen, oxygen, and sulfur.

In some embodiments, each instance of R^{22C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{22C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{22C} is independently an optionally substituted C₁₋₆ aliphatic or an optionally substituted phenyl. In some embodiments, each instance of R^{22C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{22C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{22C} is independently a C₁₋₆ aliphatic. In some embodiments, R^{22C} is phenyl. In some embodiments, each instance of R^{22C} is independently a 3-7 membered saturated or partially unsaturated monocyclic heterocyclic ring having 1-2 heteroatoms independently selected from nitrogen, oxygen, and sulfur. In some embodiments, each instance of R^{22C} is independently a 5-6 membered monocyclic heteroaryl ring having 1-4 heteroatoms independently selected from nitrogen, oxygen, and sulfur.

In some embodiments, each instance of R^{22C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{22C} is independently phenyl or a 5-6 membered monocyclic heteroaryl ring having 1-4 heteroatoms independently selected from nitrogen, oxygen, and sulfur.

In some embodiments, each instance of R^{22C} is independently a C₁₋₆ aliphatic or phenyl. In some embodiments, each instance of R^{22C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{22C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{22C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{22C} is independently halogen, —CN, —OH, —O-(optionally substituted C₁₋₃ aliphatic), or an optionally substituted C₁₋₃ aliphatic. In some embodiments, each instance of R^{22C} is independently halogen, —OH, —O—(C₁₋₃ aliphatic), or C₁₋₃ aliphatic, wherein each C₁₋₃ aliphatic is optionally substituted with 1-3 halogen. In some embodiments, each instance of R^{22C} is independently fluorine, chlorine, —OH, —OCH₃, —OCF₃, —CH₃, —CHF₂, or —CF₃. In some embodiments, each instance of R^{22C} is independently fluorine or —OH.

In some embodiments, each instance of R^{22C} is independently oxo, deuterium, halogen, —CN, —OH, —O-(optionally substituted C₁₋₃ aliphatic), or an optionally substituted C₁₋₃ aliphatic. In some embodiments, each instance of R^{22C} is independently oxo, deuterium, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{22C} is independently oxo, deuterium, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{22C} is independently oxo, deuterium, fluorine, chlorine, —CN, —OH, —OCH₃, —OCF₃, —CH₃, —CHF₂, or —CF₃. In some embodiments, each instance of R^{22C} is independently oxo, deuterium, —CN, fluorine, or —OH. In some embodiments, each instance of R^{22C} is independently oxo, deuterium, —CN, —CH₃, or —CHF₂. In some embodiments, each instance of R^{22C} is independently deuterium, —CN, —CH₃, or —CHF₂.

In some embodiments, each instance of R^{22C} is independently oxo, halogen, —CN, —OH, —O-(optionally substituted C₁₋₃ aliphatic), or an optionally substituted C₁₋₃ aliphatic. In some embodiments, each instance of R^{22C} is independently oxo, halogen, —CN, —OH, —O—(C-3 aliphatic), or C₁₋₃ aliphatic, wherein each C₁₋₃ aliphatic is optionally substituted with one or more halogen atoms. In some embodiments, each instance of R^{22C} is independently oxo, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{22C} is independently oxo, fluorine, chlorine, —CN, —OH, —OCH₃, —OCF₃, —CH₃, —CHF₂, or —CF₃. In some embodiments, each instance of R^{22C} is independently oxo, —CN, fluorine, or —OH. In some embodiments, each instance of R^{22C} is independently oxo, —CN, —CH₃, or —CHF₂. In some embodiments, each instance of R^{22C} is independently —CN, —CH₃, or —CHF₂.

In some embodiments, each instance of R^{22C} is independently selected from the groups depicted in the compounds in Table 1.

As defined generally above, each instance of R^{TC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{T1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{T1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{T1C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{T1C} is oxo. In some embodiments, R^{T1C} is deuterium. In some embodiments, each instance of R^{T1C} is independently halogen. In some embodiments, R^{T1C} is —CN. In some embodiments, R^{T1C} is —NO₂. In some embodiments, R^{T1C} is —OR. In some embodiments, R^{T1C} is —SR. In some embodiments, R^{T1C} is —NR₂. In some embodiments, R^{T1C} is —S(O)₂R. In some embodiments, R^{T1C} is —S(O)₂NR₂. In some embodiments, R^{T1C} is —S(O)₂F. In some embodiments, R^{T1C} is —S(O)R. In some embodiments, R^{T1C} is —S(O)NR₂. In some embodiments, R^{T1C} is —S(O)(NR)R. In some embodiments, R^{T1C} is —C(O)R. In some embodiments, R^{T1C} is —C(O)OR. In some embodiments, R^{T1C} is —C(O)NR₂. In some embodiments, R^{T1C} is —C(O)N(R)OR. In some embodiments, R^{T1C} is —OC(O)R. In some embodiments, R^{T1C} is —OC(O)NR₂. In some embodiments, R^{T1C} is —N(R)C(O)OR. In some embodiments, R^{T1C} is —N(R)C(O)R. In some embodiments, R^{T1C} is —N(R)C(O)NR₂. In some embodiments, R^{T1C} is —N(R)C(NR)NR₂. In some embodiments, R^{T1C} is —N(R)S(O)₂NR₂. In some embodiments, R^{T1C} is —N(R)S(O)₂R. In some embodiments, R^{T1C} is —P(O)R₂. In some embodiments, R^{T1C} is —P(O)(R)OR. In some embodiments, R^{T1C} is —B(OR)₂.

In some embodiments, each instance of R^{T1C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{T1C} is independently halogen, —CN, or —NO₂. In some embodiments, each instance of R^{T1C} is independently —OR, —SR, or —NR₂. In some embodiments, each instance of R^{T1C} is independently —S(O)₂R, —S(O)₂NR₂, —S(O)₂F, —S(O)R, —S(O)NR₂, or —S(O)(NR)R. In some embodiments, each instance of R^{TLC} is independently —C(O)R, —C(O)OR, —C(O)NR₂, or —C(O)N(R)OR. In some embodiments, each instance of R^{T1C} is independently —OC(O)R or —OC(O)NR₂. In some embodiments, each instance of R^{T1C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{T1C} is independently —P(O)R² or —P(O)(R)OR.

In some embodiments, each instance of R^{T1C} is independently —OR, —OC(O)R, or —OC(O)NR₂. In some embodiments, each instance of R^{T1C} is independently —SR, —S(O)₂R, —S(O)₂NR₂, —S(O)₂F, —S(O)R, —S(O)NR₂, or —S(O)(NR)R. In some embodiments, each instance of R^{T1C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{T1C} is independently —S(O)₂R, —S(O)₂NR₂, or —S(O)₂F. In some embodiments, each instance of R^{T1C} is independently —S(O)R, —S(O)NR₂, or —S(O)(NR)R. In some embodiments, each instance of R^{T1C} is independently —SR, —S(O)₂R, or —S(O)R. In some embodiments, each instance of R^{T1C} is independently —S(O)₂NR₂, —S(O)NR₂, or —S(O)(NR)R. In some embodiments, each instance of R^{T1C} is independently —S(O)₂NR₂ or —S(O)NR₂. In some embodiments, each instance of R^{T1C} is independently —SR, —S(O)₂R, —S(O)₂NR₂, or —S(O)R.

In some embodiments, each instance of R^{T1C} is independently —N(R)C(O)OR, —N(R)C(O)R, or —N(R)C(O)NR₂. In some embodiments, each instance of R^{T1C} is independently —N(R)S(O)₂NR₂ or —N(R)S(O)₂R. In some embodiments, each instance of R^{TLC} is independently —N(R)C(O)OR or —N(R)C(O)R. In some embodiments, each instance of R^{T1C} is independently —N(R)C(O)NR₂ or —N(R)S(O)₂NR₂. In some embodiments, each instance of R^{T1C} is independently —N(R)C(O)OR, —N(R)C(O)R, or —N(R)S(O)₂R.

In some embodiments, each instance of R^{T1C} is independently —NR₂, —N(R)C(O)OR, —N(R)C(O)R, or —N(R)C(O)NR₂. In some embodiments, each instance of R^{T1C} is independently —NR₂, —N(R)C(O)OR, or —N(R)C(O)R. In some embodiments, each instance of R^{T1C} is independently —NR₂, —N(R)C(O)OR, —N(R)C(O)R, or —N(R)S(O)₂R.

In some embodiments, each instance of R^{T1C} is independently an optionally substituted C₁₋₆ aliphatic. In some embodiments, each instance of R^{T1C} is independently an optionally substituted phenyl. In some embodiments, each instance of R^{T1C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{T1C} is independently an optionally substituted 5-6 membered monocyclic heteroaryl ring having 1-4 heteroatoms independently selected from nitrogen, oxygen, and sulfur.

In some embodiments, each instance of R^{T1C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{T1C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{T1C} is independently an optionally substituted C₁₋₆ aliphatic or an optionally substituted phenyl. In some embodiments, each instance of R^{T1C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{T1C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{TLC} is independently a C₁₋₆ aliphatic. In some embodiments, R^{T1C} is phenyl. In some embodiments, each instance of R^{T1C} is independently a 3-7 membered saturated or partially unsaturated monocyclic heterocyclic ring having 1-2 heteroatoms independently selected from nitrogen, oxygen, and sulfur. In some embodiments, each instance of R^{TLC} is independently a 5-6 membered monocyclic heteroaryl ring having 1-4 heteroatoms independently selected from nitrogen, oxygen, and sulfur.

In some embodiments, each instance of R^{T1C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{T1C} is independently phenyl or a 5-6 membered monocyclic heteroaryl ring having 1-4 heteroatoms independently selected from nitrogen, oxygen, and sulfur.

In some embodiments, each instance of R^{T1C} is independently a C₁₋₆ aliphatic or phenyl. In some embodiments, each instance of R^{T1C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{T1C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{T1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{T1C} is independently halogen, —CN, —OH, —O-(optionally substituted C₁₋₃ aliphatic), or an optionally substituted C-3 aliphatic. In some embodiments, each instance of R^{T1C} is independently halogen, —OH, —O—(C₁₋₃ aliphatic), or C₁₋₃ aliphatic, wherein each C₁₋₃ aliphatic is optionally substituted with 1-3 halogen. In some embodiments, each instance of R^{T1C} is independently fluorine, chlorine, —OH, —OCH₃, —OCF₃, —CH₃, —CHF₂, or —CF₃. In some embodiments, each instance of R^{T1C} is independently fluorine or —OH.

In some embodiments, each instance of R^{T1C} is independently oxo, deuterium, halogen, —CN, —OH, —O-(optionally substituted C₁₋₃ aliphatic), or an optionally substituted C₁₋₃ aliphatic. In some embodiments, each instance of R^{T1C} is independently oxo, deuterium, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{T1C} is independently oxo, deuterium, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{T1C} is independently oxo, deuterium, fluorine, chlorine, —CN, —OH, —OCH₃, —OCF₃, —CH₃, —CHF₂, or —CF₃. In some embodiments, each instance of R^{T1C} is independently oxo, deuterium, —CN, fluorine, or —OH. In some embodiments, each instance of R^{T1C} is independently oxo, deuterium, —CN, —CH₃, or —CHF₂. In some embodiments, each instance of R^{T1C} is independently deuterium, —CN, —CH₃, or —CHF₂.

In some embodiments, each instance of R^{T1C} is independently oxo, halogen, —CN, —OH, —O-(optionally substituted C₁₋₃ aliphatic), or an optionally substituted C₁₋₃ aliphatic. In some embodiments, each instance of R^{TLC} is independently oxo, halogen, —CN, —OH, —O—(C-3 aliphatic), or C₁₋₃ aliphatic, wherein each C-3 aliphatic is optionally substituted with one or more halogen atoms. In some embodiments, each instance of R^{T1C} is independently oxo, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{T1C} is independently oxo, fluorine, chlorine, —CN, —OH, —OCH₃, —OCF₃, —CH₃, —CHF₂, or —CF₃. In some embodiments, each instance of R^{TLC} is independently oxo, —CN, fluorine, or —OH. In some embodiments, each instance of R^{TLC} is independently oxo, —CN, —CH₃, or —CHF₂. In some embodiments, each instance of R^{TLC} is independently —CN, —CH₃, or —CHF₂.

In some embodiments, each instance of R^{TLC} is independently selected from the groups depicted in the compounds in Table 1.

As defined generally above, each instance of R^{TLC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R², —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{TLC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{TLC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R², —P(O)(R)OR, or —B(OR)₂. In some embodiments, each instance of R^{TLC} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{TLC} is OXO. In some embodiments, R^{TLC} is deuterium. In some embodiments, each instance of R^{TLC} is independently halogen. In some embodiments, R^{TLC} is —CN. In some embodiments, R^{TLC} is —NO₂. In some embodiments, R^{TLC} is —OR. In some embodiments, R^{TLC} is —SR. In some embodiments, R^{TLC} is —NR₂. In some embodiments, R^{TLC} is —S(O)₂R. In some embodiments, R^{TLC} is —S(O)₂NR₂. In some embodiments, R^{TLC} is —S(O)₂F. In some embodiments, R^{TLC} is —S(O)R. In some embodiments, R^{TLC} is —S(O)NR₂. In some embodiments, R^{TLC} is —S(O)(NR)R. In some embodiments, R^{TLC} is —C(O)R. In some embodiments, R^{TLC} is —C(O)OR. In some embodiments, R^{TLC} is —C(O)NR₂. In some embodiments, R^{TLC} is —C(O)N(R)OR. In some embodiments, R^{TLC} is —OC(O)R. In some embodiments, R^{TLC} is —OC(O)NR₂. In some embodiments, R^{TLC} is —N(R)C(O)OR. In some embodiments, R^{TLC} is —N(R)C(O)R. In some embodiments, R^{TLC} is —N(R)C(O)NR₂. In some embodiments, R^{TLC} is —N(R)C(NR)NR₂. In some embodiments, R^{TLC} is —N(R)S(O)₂NR₂. In some embodiments, R^{TLC} is —N(R)S(O)₂R. In some embodiments, R^{TLC} is —P(O)R₂. In some embodiments, R^{TLC} is —P(O)(R)OR. In some embodiments, R^{TLC} is —B(OR)₂.

In some embodiments, each instance of R^{TLC} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{TLC} is independently halogen, —CN, or —NO₂. In some embodiments, each instance of R^{TLC} is independently —OR, —SR, or —NR₂. In some embodiments, each instance of R^{TLC} is independently —S(O)₂R, —S(O)₂NR₂, —S(O)₂F, —S(O)R, —S(O)NR₂, or —S(O)(NR)R. In some embodiments, each instance of R^{TLC} is independently —C(O)R, —C(O)OR, —C(O)NR₂, or —C(O)N(R)OR. In some embodiments, each instance of R^{TLC} is independently —OC(O)R or —OC(O)NR₂. In some embodiments, each instance of R^{TLC} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{TLC} is independently —P(O)R₂ or —P(O)(R)OR.

In some embodiments, each instance of R^{TLC} is independently —OR, —OC(O)R, or —OC(O)NR₂. In some embodiments, each instance of R^{TLC} is independently —SR, —S(O)₂R, —S(O)₂NR₂, —S(O)₂F, —S(O)R, —S(O)NR₂, or —S(O)(NR)R. In some embodiments, each instance of R^{TLC} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{TLC} is independently —S(O)₂R, —S(O)₂NR₂, or —S(O)₂F. In some embodiments, each instance of R^{TLC} is independently —S(O)R, —S(O)NR₂, or —S(O)(NR)R. In some embodiments, each instance of R^{TLC} is independently —SR, —S(O)₂R, or —S(O)R. In some embodiments, each instance of R^{TLC} is independently —S(O)₂NR₂, —S(O)NR₂, or —S(O)(NR)R. In some embodiments, each instance of R^{TLC} is independently —S(O)₂NR₂ or —S(O)NR₂. In some embodiments, each instance of R^{TLC} is independently —SR, —S(O)₂R, —S(O)₂NR₂, or —S(O)R.

In some embodiments, each instance of R^{TLC} is independently —N(R)C(O)OR, —N(R)C(O)R, or —N(R)C(O)NR₂. In some embodiments, each instance of R^{TLC} is independently —N(R)S(O)₂NR₂ or —N(R)S(O)₂R. In some embodiments, each instance of R^{TLC} is independently —N(R)C(O)OR or —N(R)C(O)R. In some embodiments, each instance of R^{TLC} is independently —N(R)C(O)NR₂ or —N(R)S(O)₂NR₂. In some embodiments, each instance of R^{TLC} is independently —N(R)C(O)OR, —N(R)C(O)R, or —N(R)S(O)₂R.

In some embodiments, each instance of R^{TLC} is independently —NR₂, —N(R)C(O)OR, —N(R)C(O)R, or —N(R)C(O)NR₂. In some embodiments, each instance of R^{TLC} is independently —NR₂, —N(R)C(O)OR, or —N(R)C(O)R. In some embodiments, each instance of R^{TLC} is independently —NR₂, —N(R)C(O)OR, —N(R)C(O)R, or —N(R)S(O)₂R.

In some embodiments, each instance of R^{TLC} is independently an optionally substituted C₁₋₆ aliphatic. In some embodiments, each instance of R^{TLC} is independently an optionally substituted phenyl. In some embodiments, each instance of R^{TLC} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{TLC} is independently an optionally substituted 5-6 membered monocyclic heteroaryl ring having 1-4 heteroatoms independently selected from nitrogen, oxygen, and sulfur.

In some embodiments, each instance of R^{TLC} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{TLC} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{TLC} is independently an optionally substituted C₁₋₆ aliphatic or an optionally substituted phenyl. In some embodiments, each instance of R^{TLC} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{TLC} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{TLC} is independently a C₁₋₆ aliphatic. In some embodiments, R^{TLC} is phenyl. In some embodiments, each instance of R^{TLC} is independently a 3-7 membered saturated or partially unsaturated monocyclic heterocyclic ring having 1-2 heteroatoms independently selected from nitrogen, oxygen, and sulfur. In some embodiments, each instance of R^{TLC} is independently a 5-6 membered monocyclic heteroaryl ring having 1-4 heteroatoms independently selected from nitrogen, oxygen, and sulfur.

In some embodiments, each instance of R^{TLC} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{TLC} is independently phenyl or a 5-6 membered monocyclic heteroaryl ring having 1-4 heteroatoms independently selected from nitrogen, oxygen, and sulfur.

In some embodiments, each instance of R^{TLC} is independently a C₁₋₆ aliphatic or phenyl. In some embodiments, each instance of R^{TLC} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{TLC} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{TLC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{TLC} is independently halogen, —CN, —OH, —O-(optionally substituted C₁₋₃ aliphatic), or an optionally substituted C₁₋₃ aliphatic. In some embodiments, each instance of R^{TLC} is independently halogen, —OH, —O—(C₁₋₃ aliphatic), or C₁₋₃ aliphatic, wherein each C₁₋₃ aliphatic is optionally substituted with 1-3 halogen. In some embodiments, each instance of R^{TLC} is independently fluorine, chlorine, —OH, —OCH₃, —OCF₃, —CH₃, —CHF₂, or —CF₃. In some embodiments, each instance of R^{TLC} is independently fluorine or —OH.

In some embodiments, each instance of R^{TLC} is independently oxo, deuterium, halogen, —CN, —OH, —O-(optionally substituted C₁₋₃ aliphatic), or an optionally substituted C₁₋₃ aliphatic. In some embodiments, each instance of R^{TLC} is independently oxo, deuterium, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{TLC} is independently oxo, deuterium, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{TLC} is independently oxo, deuterium, fluorine, chlorine, —CN, —OH, —OCH₃, —OCF₃, —CH₃, —CHF₂, or —CF₃. In some embodiments, each instance of R^{TLC} is independently oxo, deuterium, —CN, fluorine, or —OH. In some embodiments, each instance of R^{TLC} is independently oxo, deuterium, —CN, —CH₃, or —CHF₂. In some embodiments, each instance of R^{TLC} is independently deuterium, —CN, —CH₃, or —CHF₂.

In some embodiments, each instance of R^{TLC} is independently oxo, halogen, —CN, —OH, —O-(optionally substituted C₁₋₃ aliphatic), or an optionally substituted C₁₋₃ aliphatic. In some embodiments, each instance of R^{TLC} is independently oxo, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{TLC} is independently oxo, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{TLC} is independently oxo, fluorine, chlorine, —CN, —OH, —OCH₃, —OCF₃, —CH₃, —CHF₂, or —CF₃. In some embodiments, each instance of R^{TLC} is independently oxo, —CN, fluorine, or —OH. In some embodiments, each instance of R^{TLC} is independently oxo, —CN, —CH₃, or —CHF₂. In some embodiments, each instance of R^{TLC} is independently —CN, —CH₃, or —CHF₂.

In some embodiments, each instance of R^{TLC} is independently selected from the groups depicted in the compounds in Table 1.

As defined generally above, each instance of R^{LC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{LC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{LC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{LC} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{LC} is oxo. In some embodiments, R^{LC} is deuterium. In some embodiments, each instance of R^{LC} is independently halogen. In some embodiments, R^{LC} is —CN. In some embodiments, R^{LC} is —NO₂. In some embodiments, R^{LC} is —OR. In some embodiments, R^{LC} is —SR. In some embodiments, R^{LC} is —NR₂. In some embodiments, R^{LC} is —S(O)₂R. In some embodiments, R^{LC} is —S(O)₂NR₂. In some embodiments, R^{LC} is —S(O)₂F. In some embodiments, R^{LC} is —S(O)R. In some embodiments, R^{LC} is —S(O)NR₂. In some embodiments, R^{LC} is —S(O)(NR)R. In some embodiments, R^{LC} is —C(O)R. In some embodiments, R^{LC} is —C(O)OR. In some embodiments, R^{LC} is —C(O)NR₂. In some embodiments, R^{LC} is —C(O)N(R)OR. In some embodiments, R^{LC} is —OC(O)R. In some embodiments, R^{LC} is —OC(O)NR₂. In some embodiments, R^{LC} is —N(R)C(O)OR. In some embodiments, R^{LC} is —N(R)C(O)R. In some embodiments, R^{LC} is —N(R)C(O)NR₂. In some embodiments, R^{LC} is —N(R)C(NR)NR₂. In some embodiments, R^{LC} is —N(R)S(O)₂NR₂. In some embodiments, R^{LC} is —N(R)S(O)₂R. In some embodiments, R^{LC} is —P(O)R₂. In some embodiments, R^{LC} is —P(O)(R)OR. In some embodiments, R^{LC} is —B(OR)₂.

In some embodiments, each instance of R^{LC} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{LC} is independently halogen, —CN, or —NO₂. In some embodiments, each instance of R^{LC} is independently —OR, —SR, or —NR₂. In some embodiments, each instance of R^{LC} is independently —S(O)₂R, —S(O)₂NR₂, —S(O)₂F, —S(O)R, —S(O)NR₂, or —S(O)(NR)R. In some embodiments, each instance of R^{LC} is independently —C(O)R, —C(O)OR, —C(O)NR₂, or —C(O)N(R)OR. In some embodiments, each instance of R^{LC} is independently —OC(O)R or —OC(O)NR₂. In some embodiments, each instance of R^{LC} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{LC} is independently —P(O)R₂ or —P(O)(R)OR.

In some embodiments, each instance of R^{LC} is independently —OR, —OC(O)R, or —OC(O)NR₂. In some embodiments, each instance of R^{LC} is independently —SR, —S(O)₂R, —S(O)₂NR₂, —S(O)₂F, —S(O)R, —S(O)NR₂, or —S(O)(NR)R. In some embodiments, each instance of R^{LC} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{LC} is independently —S(O)₂R, —S(O)₂NR₂, or —S(O)₂F. In some embodiments, each instance of R^{LC} is independently —S(O)R, —S(O)NR₂, or —S(O)(NR)R. In some embodiments, each instance of R^{LC} is independently —SR, —S(O)₂R, or —S(O)R. In some embodiments, each instance of R^{LC} is independently —S(O)₂NR₂, —S(O)NR₂, or —S(O)(NR)R. In some embodiments, each instance of R^{LC} is independently —S(O)₂NR₂ or —S(O)NR₂. In some embodiments, each instance of R^{LC} is independently —SR, —S(O)₂R, —S(O)₂NR₂, or —S(O)R.

In some embodiments, each instance of R^{LC} is independently —N(R)C(O)OR, —N(R)C(O)R, or —N(R)C(O)NR₂. In some embodiments, each instance of R^{LC} is independently —N(R)S(O)₂NR₂ or —N(R)S(O)₂R. In some embodiments, each instance of R^{LC} is independently —N(R)C(O)OR or —N(R)C(O)R. In some embodiments, each instance of R^{LC} is independently —N(R)C(O)NR₂ or —N(R)S(O)₂NR₂. In some embodiments, each instance of R^{LC} is independently —N(R)C(O)OR, —N(R)C(O)R, or —N(R)S(O)₂R.

In some embodiments, each instance of R^{LC} is independently —NR₂, —N(R)C(O)OR, —N(R)C(O)R, or —N(R)C(O)NR₂. In some embodiments, each instance of R^{LC} is independently —NR₂, —N(R)C(O)OR, or —N(R)C(O)R. In some embodiments, each instance of R^{LC} is independently —NR₂, —N(R)C(O)OR, —N(R)C(O)R, or —N(R)S(O)₂R.

In some embodiments, each instance of R^{LC} is independently an optionally substituted C₁₋₆ aliphatic. In some embodiments, each instance of R^{LC} is independently an optionally substituted phenyl. In some embodiments, each instance of R^{LC} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{LC} is independently an optionally substituted 5-6 membered monocyclic heteroaryl ring having 1-4 heteroatoms independently selected from nitrogen, oxygen, and sulfur.

In some embodiments, each instance of R^{LC} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{LC} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{LC} is independently an optionally substituted C₁₋₆ aliphatic or an optionally substituted phenyl. In some embodiments, each instance of R^{LC} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{LC} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{LC} is independently a C₁₋₆ aliphatic. In some embodiments, R^{LC} is phenyl. In some embodiments, each instance of R^{LC} is independently a 3-7 membered saturated or partially unsaturated monocyclic heterocyclic ring having 1-2 heteroatoms independently selected from nitrogen, oxygen, and sulfur. In some embodiments, each instance of R^{LC} is independently a 5-6 membered monocyclic heteroaryl ring having 1-4 heteroatoms independently selected from nitrogen, oxygen, and sulfur.

In some embodiments, each instance of R^{LC} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{LC} is independently phenyl or a 5-6 membered monocyclic heteroaryl ring having 1-4 heteroatoms independently selected from nitrogen, oxygen, and sulfur.

In some embodiments, each instance of R^{LC} is independently a C₁₋₆ aliphatic or phenyl. In some embodiments, each instance of R^{LC} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{LC} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{LC} is independently selected from the groups depicted in the compounds in Table 1.

As defined generally above, each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur. In some embodiments, two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen. In some embodiments, R is an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur. In some embodiments, R is hydrogen, C₁₋₆ aliphatic, phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, R is an optionally substituted C₁₋₆ aliphatic. In some embodiments, R is an optionally substituted phenyl. In some embodiments, R is an optionally substituted 3-7 membered saturated or partially unsaturated monocyclic heterocyclic ring having 1-2 heteroatoms independently selected from nitrogen, oxygen, and sulfur. In some embodiments, R is an optionally substituted 5-6 membered monocyclic heteroaryl ring having 1-4 heteroatoms independently selected from nitrogen, oxygen, and sulfur.

In some embodiments, R is an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, R is an optionally substituted phenyl or an optionally substituted 5-6 membered monocyclic heteroaryl ring having 1-4 heteroatoms independently selected from nitrogen, oxygen, and sulfur.

In some embodiments, R is an optionally substituted C₁₋₆ aliphatic or an optionally substituted phenyl. In some embodiments, R is an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, R is an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R is a C₁₋₆ aliphatic. In some embodiments, R is phenyl. In some embodiments, R is a 3-7 membered saturated or partially unsaturated monocyclic heterocyclic ring having 1-2 heteroatoms independently selected from nitrogen, oxygen, and sulfur. In some embodiments, R is a 5-6 membered monocyclic heteroaryl ring having 1-4 heteroatoms independently selected from nitrogen, oxygen, and sulfur.

In some embodiments, R is a C₁₋₆ aliphatic or a 3-7 membered saturated or partially unsaturated monocyclic heterocyclic ring having 1-2 heteroatoms independently selected from nitrogen, oxygen, and sulfur. In some embodiments, R is phenyl or a 5-6 membered monocyclic heteroaryl ring having 1-4 heteroatoms independently selected from nitrogen, oxygen, and sulfur.

In some embodiments, R is a C₁₋₆ aliphatic or phenyl. In some embodiments, R is a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, R is phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, two R groups on the same nitrogen are taken together with their intervening atoms to form a 4-7 membered saturated, partially unsaturated, or heteroaryl ring having 1-3 heteroatoms, in addition to the nitrogen, independently selected from nitrogen, oxygen, and sulfur. In some embodiments, two R groups on the same nitrogen are taken together with their intervening atoms to form a 4-7 membered saturated, partially unsaturated, or heteroaryl ring having no additional heteroatoms other than said nitrogen.

In some embodiments, two R groups on the same nitrogen are taken together with their intervening atoms to form a 4-7 membered saturated ring having 0-3 heteroatoms, in addition to the nitrogen, independently selected from nitrogen, oxygen, and sulfur. In some embodiments, two R groups on the same nitrogen are taken together with their intervening atoms to form a 4-7 membered partially unsaturated ring having 0-3 heteroatoms, in addition to the nitrogen, independently selected from nitrogen, oxygen, and sulfur. In some embodiments, two R groups on the same nitrogen are taken together with their intervening atoms to form a 4-7 membered heteroaryl ring having 0-3 heteroatoms, in addition to the nitrogen, independently selected from nitrogen, oxygen, and sulfur.

In some embodiments, two R groups on the same nitrogen are taken together with their intervening atoms to form a 4-7 membered saturated ring having 1-3 heteroatoms, in addition to the nitrogen, independently selected from nitrogen, oxygen, and sulfur. In some embodiments, two R groups on the same nitrogen are taken together with their intervening atoms to form a 4-7 membered partially unsaturated ring having 1-3 heteroatoms, in addition to the nitrogen, independently selected from nitrogen, oxygen, and sulfur. In some embodiments, two R groups on the same nitrogen are taken together with their intervening atoms to form a 4-7 membered heteroaryl ring having 1-3 heteroatoms, in addition to the nitrogen, independently selected from nitrogen, oxygen, and sulfur.

In some embodiments, two R groups on the same nitrogen are taken together with their intervening atoms to form a 4-7 membered saturated ring having no additional heteroatoms other than said nitrogen. In some embodiments, two R groups on the same nitrogen are taken together with their intervening atoms to form a 4-7 membered partially unsaturated ring having no additional heteroatoms other than said nitrogen. In some embodiments, two R groups on the same nitrogen are taken together with their intervening atoms to form a 4-7 membered heteroaryl ring having no additional heteroatoms other than said nitrogen.

In some embodiments, R is selected from the groups depicted in the compounds in Table 1.

As defined generally above, n is 0, 1, 2, 3, 4, or 5. In some embodiments, n is 0. In some embodiments, n is 1. In some embodiments, n is 2. In some embodiments, n is 3. In some embodiments, n is 4. In some embodiments, n is 5. In some embodiments, n is 0 or 1. In some embodiments, n is 0, 1, or 2. In some embodiments, n is 0, 1, 2, or 3. In some embodiments, n is 0, 1, 2, 3, or 4. In some embodiments, n is 1 or 2. In some embodiments, n is 1, 2, or 3. In some embodiments, n is 1, 2, 3, or 4. In some embodiments, n is 1, 2, 3, 4, or 5. In some embodiments, n is 2 or 3. In some embodiments, n is 2, 3, or 4. In some embodiments, n is 2, 3, 4, or 5. In some embodiments, n is 3 or 4. In some embodiments, n is 3, 4, or 5. In some embodiments, n is 4 or 5. In some embodiments, n is selected from the values represented in the compounds in Table 1.

As defined generally above, m is 0, 1, 2, 3, 4, or 5. In some embodiments, m is 0. In some embodiments, m is 1. In some embodiments, m is 2. In some embodiments, m is 3. In some embodiments, m is 4. In some embodiments, m is 5. In some embodiments, m is 0 or 1. In some embodiments, m is 0, 1, or 2. In some embodiments, m is 0, 1, 2, or 3. In some embodiments, m is 0, 1, 2, 3, or 4. In some embodiments, m is 1 or 2. In some embodiments, m is 1, 2, or 3. In some embodiments, m is 1, 2, 3, or 4. In some embodiments, m is 1, 2, 3, 4, or 5. In some embodiments, m is 2 or 3. In some embodiments, m is 2, 3, or 4. In some embodiments, m is 2, 3, 4, or 5. In some embodiments, m is 3 or 4. In some embodiments, m is 3, 4, or 5. In some embodiments, m is 4 or 5. In some embodiments, m is selected from the values represented in the compounds in Table 1.

As defined generally above, q is 0, 1, 2, 3, 4, or 5. In some embodiments, q is 0. In some embodiments, q is 1. In some embodiments, q is 2. In some embodiments, q is 3. In some embodiments, q is 4. In some embodiments, q is 5. In some embodiments, q is 0 or 1. In some embodiments, q is 0, 1, or 2. In some embodiments, q is 0, 1, 2, or 3. In some embodiments, q is 0, 1, 2, 3, or 4. In some embodiments, q is 1 or 2. In some embodiments, q is 1, 2, or 3. In some embodiments, q is 1, 2, 3, or 4. In some embodiments, q is 1, 2, 3, 4, or 5. In some embodiments, q is 2 or 3. In some embodiments, q is 2, 3, or 4. In some embodiments, q is 2, 3, 4, or 5. In some embodiments, q is 3 or 4. In some embodiments, q is 3, 4, or 5. In some embodiments, q is 4 or 5. In some embodiments, q is selected from the values represented in the compounds in Table 1.

As defined generally above, p¹ is 0, 1, 2, 3, 4, or 5. In some embodiments, p¹ is 0. In some embodiments, p¹ is 1. In some embodiments, p¹ is 2. In some embodiments, p¹ is 3. In some embodiments, p¹ is 4. In some embodiments, p¹ is 5. In some embodiments, p¹ is 0 or 1. In some embodiments, p¹ is 0, 1, or 2. In some embodiments, p¹ is 0, 1, 2, or 3. In some embodiments, p¹ is 0, 1, 2, 3, or 4. In some embodiments, p¹ is 1 or 2. In some embodiments, p¹ is 1, 2, or 3. In some embodiments, p¹ is 1, 2, 3, or 4. In some embodiments, p¹ is 1, 2, 3, 4, or 5. In some embodiments, p¹ is 2 or 3. In some embodiments, p¹ is 2, 3, or 4. In some embodiments, p¹ is 2, 3, 4, or 5. In some embodiments, p¹ is 3 or 4. In some embodiments, p¹ is 3, 4, or 5. In some embodiments, p¹ is selected from the values represented in the compounds in Table 1.

As defined generally above, p² is 0, 1, 2, 3, 4, or 5. In some embodiments, p² is 0. In some embodiments, p² is 1. In some embodiments, p2 is 2. In some embodiments, p² is 3. In some embodiments, p² is 4. In some embodiments, p² is 5. In some embodiments, p² is 0 or 1. In some embodiments, p² is 0, 1, or 2. In some embodiments, p² is 0, 1, 2, or 3. In some embodiments, p² is 0, 1, 2, 3, or 4. In some embodiments, p² is 1 or 2. In some embodiments, p² is 1, 2, or 3. In some embodiments, p² is 1, 2, 3, or 4. In some embodiments, p² is 1, 2, 3, 4, or 5. In some embodiments, p² is 2 or 3. In some embodiments, p² is 2, 3, or 4. In some embodiments, p² is 2, 3, 4, or 5. In some embodiments, p² is 3 or 4. In some embodiments, p² is 3, 4, or 5. In some embodiments, p2 is selected from the values represented in the compounds in Table 1.

As defined generally above, p³ is 0, 1, 2, 3, 4, or 5. In some embodiments, p³ is 0. In some embodiments, p³ is 1. In some embodiments, p³ is 2. In some embodiments, p³ is 3. In some embodiments, p³ is 4. In some embodiments, p³ is 5. In some embodiments, p³ is 0 or 1. In some embodiments, p³ is 0, 1, or 2. In some embodiments, p³ is 0, 1, 2, or 3. In some embodiments, p³ is 0, 1, 2, 3, or 4. In some embodiments, p³ is 1 or 2. In some embodiments, p³ is 1, 2, or 3. In some embodiments, p³ is 1, 2, 3, or 4. In some embodiments, p³ is 1, 2, 3, 4, or 5. In some embodiments, p³ is 2 or 3. In some embodiments, p³ is 2, 3, or 4. In some embodiments, p³ is 2, 3, 4, or 5. In some embodiments, p³ is 3 or 4. In some embodiments, p³ is 3, 4, or 5. In some embodiments, p³ is selected from the values represented in the compounds in Table 1.

As defined generally above, p⁴ is 0, 1, 2, 3, 4, or 5. In some embodiments, p⁴ is 0. In some embodiments, p⁴ is 1. In some embodiments, p⁴ is 2. In some embodiments, p⁴ is 3. In some embodiments, p⁴ is 4. In some embodiments, p⁴ is 5. In some embodiments, p⁴ is 0 or 1. In some embodiments, p⁴ is 0, 1, or 2. In some embodiments, p⁴ is 0, 1, 2, or 3. In some embodiments, p⁴ is 0, 1, 2, 3, or 4. In some embodiments, p⁴ is 1 or 2. In some embodiments, p⁴ is 1, 2, or 3. In some embodiments, p⁴ is 1, 2, 3, or 4. In some embodiments, p⁴ is 1, 2, 3, 4, or 5. In some embodiments, p⁴ is 2 or 3. In some embodiments, p⁴ is 2, 3, or 4. In some embodiments, p⁴ is 2, 3, 4, or 5. In some embodiments, p⁴ is 3 or 4. In some embodiments, p⁴ is 3, 4, or 5. In some embodiments, p⁴ is selected from the values represented in the compounds in Table 1.

As defined generally above, r¹ is 0, 1, 2, 3, 4, or 5. In some embodiments, r¹ is 0. In some embodiments, r¹ is 1. In some embodiments, r¹ is 2. In some embodiments, r¹ is 3. In some embodiments, r¹ is 4. In some embodiments, r¹ is 5. In some embodiments, r¹ is 0 or 1. In some embodiments, r¹ is 0, 1, or 2. In some embodiments, r¹ is 0, 1, 2, or 3. In some embodiments, r¹ is 0, 1, 2, 3, or 4. In some embodiments, r¹ is 1 or 2. In some embodiments, r¹ is 1, 2, or 3. In some embodiments, r¹ is 1, 2, 3, or 4. In some embodiments, r¹ is 1, 2, 3, 4, or 5. In some embodiments, r¹ is 2 or 3. In some embodiments, r¹ is 2, 3, or 4. In some embodiments, r¹ is 2, 3, 4, or 5. In some embodiments, r¹ is 3 or 4. In some embodiments, r¹ is 3, 4, or 5. In some embodiments, r¹ is selected from the values represented in the compounds in Table 1.

As defined generally above, r² is 0, 1, 2, 3, 4, or 5. In some embodiments, r² is 0. In some embodiments, r² is 1. In some embodiments, r² is 2. In some embodiments, r² is 3. In some embodiments, r² is 4. In some embodiments, r² is 5. In some embodiments, r² is 0 or 1. In some embodiments, r² is 0, 1, or 2. In some embodiments, r² is 0, 1, 2, or 3. In some embodiments, r² is 0, 1, 2, 3, or 4. In some embodiments, r² is 1 or 2. In some embodiments, r² is 1, 2, or 3. In some embodiments, r² is 1, 2, 3, or 4. In some embodiments, r² is 1, 2, 3, 4, or 5. In some embodiments, r² is 2 or 3. In some embodiments, r² is 2, 3, or 4. In some embodiments, r² is 2, 3, 4, or 5. In some embodiments, r² is 3 or 4. In some embodiments, r² is 3, 4, or 5. In some embodiments, r² is selected from the values represented in the compounds in Table 1.

As defined generally above, r³ is 0, 1, 2, 3, 4, or 5. In some embodiments, r³ is 0. In some embodiments, r³ is 1. In some embodiments, r³ is 2. In some embodiments, r³ is 3. In some embodiments, r³ is 4. In some embodiments, r³ is 5. In some embodiments, r³ is 0 or 1. In some embodiments, r³ is 0, 1, or 2. In some embodiments, r³ is 0, 1, 2, or 3. In some embodiments, r³ is 0, 1, 2, 3, or 4. In some embodiments, r³ is 1 or 2. In some embodiments, r³ is 1, 2, or 3. In some embodiments, r³ is 1, 2, 3, or 4. In some embodiments, r³ is 1, 2, 3, 4, or 5. In some embodiments, r³ is 2 or 3. In some embodiments, r³ is 2, 3, or 4. In some embodiments, r³ is 2, 3, 4, or 5. In some embodiments, r³ is 3 or 4. In some embodiments, r³ is 3, 4, or 5. In some embodiments, r³ is selected from the values represented in the compounds in Table 1.

As defined generally above, r⁴ is 0, 1, 2, 3, 4, or 5. In some embodiments, r⁴ is 0. In some embodiments, r⁴ is 1. In some embodiments, r⁴ is 2. In some embodiments, r⁴ is 3. In some embodiments, r⁴ is 4. In some embodiments, r⁴ is 5. In some embodiments, r⁴ is 0 or 1. In some embodiments, r⁴ is 0, 1, or 2. In some embodiments, r⁴ is 0, 1, 2, or 3. In some embodiments, r⁴ is 0, 1, 2, 3, or 4. In some embodiments, r⁴ is 1 or 2. In some embodiments, r⁴ is 1, 2, or 3. In some embodiments, r⁴ is 1, 2, 3, or 4. In some embodiments, r⁴ is 1, 2, 3, 4, or 5. In some embodiments, r⁴ is 2 or 3. In some embodiments, r⁴ is 2, 3, or 4. In some embodiments, r⁴ is 2, 3, 4, or 5. In some embodiments, r⁴ is 3 or 4. In some embodiments, r⁴ is 3, 4, or 5. In some embodiments, r⁴ is selected from the values represented in the compounds in Table 1.

In some embodiments, the present disclosure provides a compound of formula I, wherein Cy¹ is phenyl substituted with n instances of R¹, forming a compound of formula II:

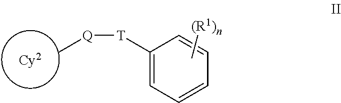

or a pharmaceutically acceptable salt thereof, wherein each of Cy², Q, R¹, T and n is as defined in embodiments and classes and subclasses herein.

In some embodiments, the present disclosure provides a compound of formula II wherein Q is —C(O)NH— or —NH—, forming a compound of formula III or IV:

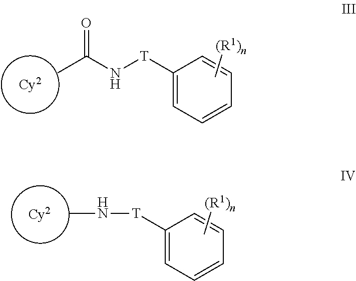

or a pharmaceutically acceptable salt thereof, wherein each of Cy², R¹, T and n is as defined in embodiments and classes and subclasses herein.

In some embodiments, the present disclosure provides a compound of formula III or IV, wherein T is selected from embodiments herein, forming a compound of formula V, VI, VII, VIII, IX, or X:

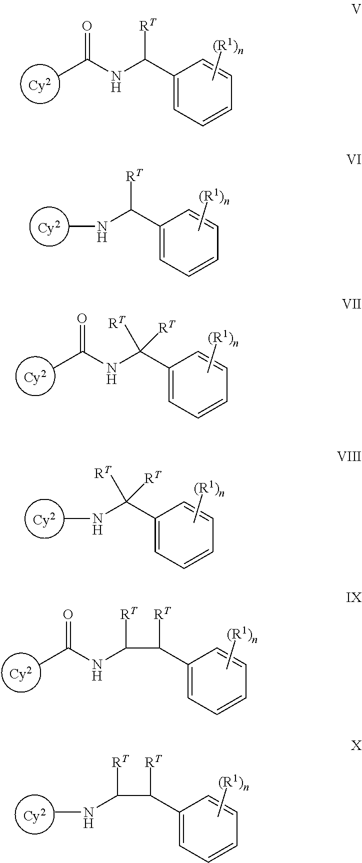

or a pharmaceutically acceptable salt thereof, wherein each of Cy², R¹, R^{T} and n is as defined in embodiments and classes and subclasses herein.

In some embodiments, the present disclosure provides a compound of formula V, wherein R^{T} is selected from embodiments herein, forming a compound of formula XI, XII, XIII, XIV, XV, XVI, XVII, XVIII, or XIX:

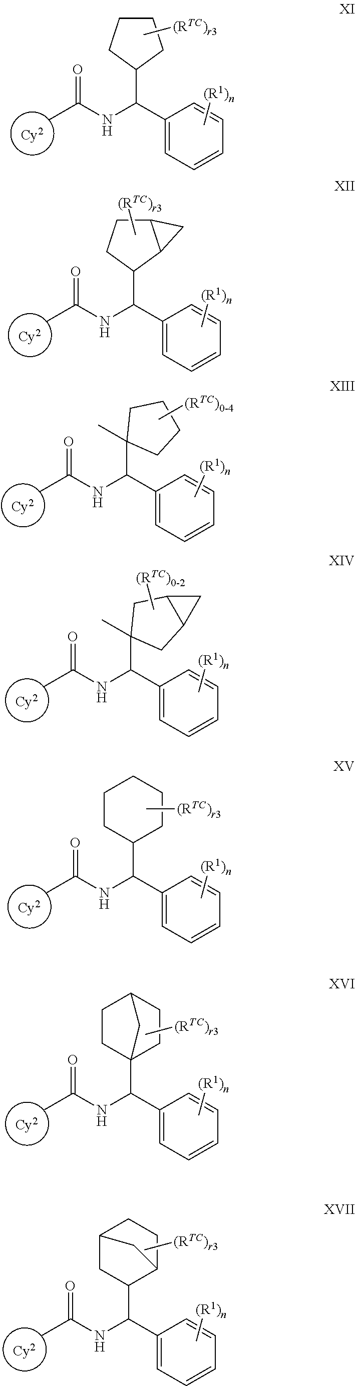

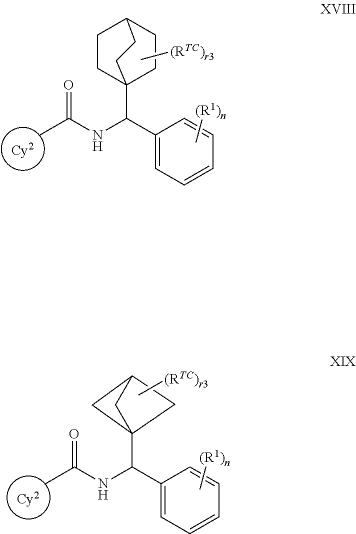

or a pharmaceutically acceptable salt thereof, wherein each of Cy², R¹, R^{TC}, n and r³ is as defined in embodiments and classes and subclasses herein.

In some embodiments, the present disclosure provides a compound of formula V, wherein Cy² is selected from embodiments herein, forming a compound of formula XX, XXI, XXII, XXIII, XXIV, or XXV:

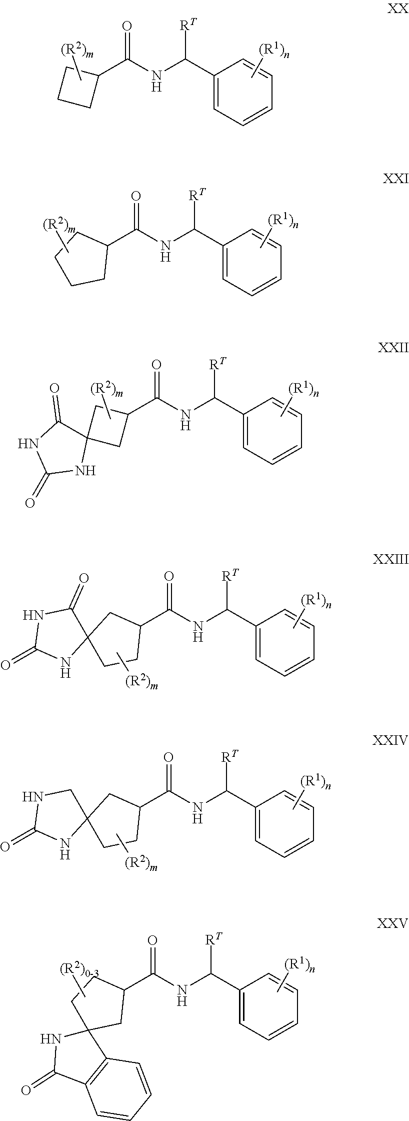

or a pharmaceutically acceptable salt thereof, wherein each of R¹, R², R^{T}, n and m is as defined in embodiments and classes and subclasses herein.

In some embodiments, the present disclosure provides a compound of formula V, wherein n and the position(s) of R¹ are selected from embodiments of Cy¹ herein, forming a compound of formulas XXVI, XXVII, XXVIII, XXIX, XXX, XXXI, XXXII, XXXIII, XXXIV, XXXV, and XXXVI:

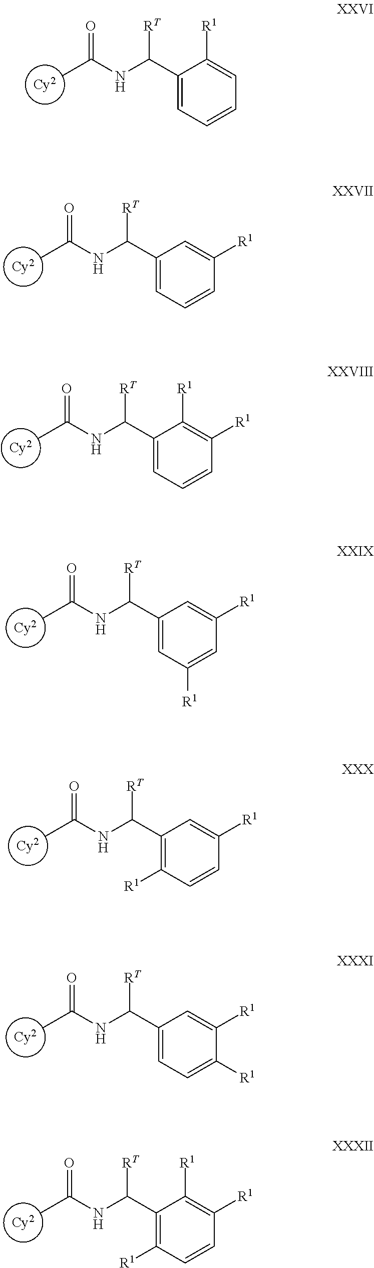

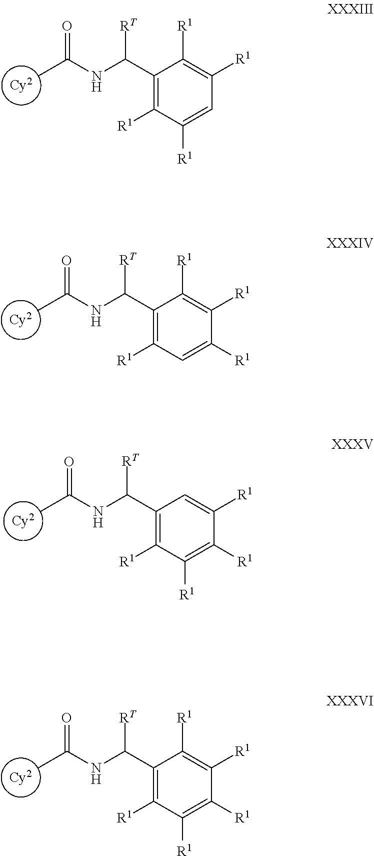

or a pharmaceutically acceptable salt thereof, wherein each of Cy², R¹, and R^{T} is as defined in embodiments and classes and subclasses herein.

In some embodiments, the present disclosure provides a compound of formula I, II, III, TV, V, VI, VII, VIII, IX, X, XI, XII, XIII, XIV, XV, XVI, XVII, XVIII, XIX, XX, XXI, XXI, XXII, XXIII, XXIV, XXV, XXVI, XXVII, XXVIII, XXIX, XXX, XXXI, XXXII, XXXIII, XXXIV, XXXV, or XXXVI, wherein L¹ is a covalent bond, and R² is —N(H)C(O)—R^{2A}, —N(H)—R^{2A}, —CH₂—R^{2A}, or —R^{2A}.

In some embodiments, the present disclosure provides a compound of I, II, III, IV, V, VI, VII, VIII, IX, X, XI, XII, XIII, XIV, XV, XVI, XVII, XVIII, XIX, XX, XXI, XXI, XXII, XXIII, XXIV, XXV, XXVI, XXVII, XXVIII, XXIX, XXX, XXXI, XXXII, XXXIII, XXXIV, XXXV, or XXXVI, wherein L¹ is a covalent bond, and R² is —N(H)C(O)—R^{2A}. In some embodiments, the present disclosure provides a compound of I, II, III, IV, V, VI, VII, VIII, IX, X, XI, XII, XIII, XIV, XV, XVI, XVII, XVIII, XIX, XX, XXI, XXI, XXII, XXIII, XXIV, XXV, XXVI, XXVII, XXVIII, XXIX, XXX, XXXI, XXXII, XXXIII, XXXIV, XXXV, or XXXVI, wherein L¹ is a covalent bond, and R² is —N(H)—R^{2A}. In some embodiments, the present disclosure provides a compound of I, II, III, IV, V, VI, VII, VIII, IX, X, XI, XII, XIII, XIV, XV, XVI, XVII, XVIII, XIX, XX, XXI, XXI, XXII, XXIII, XXIV, XXV, XXVI, XXVII, XXVIII, XXIX, XXX, XXXI, XXXII, XXXIII, XXXIV, XXXV, or XXXVI, wherein L¹ is a covalent bond, and R² is —CH₂—R^{2A}. In some embodiments, the present disclosure provides a compound of T, II, IT, TV, V, VT, VII, VIII, IX, X, XI, XII, XIII, XIV, XV, XVI, XVII, XVIII, XIX, XX, XXI, XXI, XXII, XXIII, XXIV, XXV, XXVI, XXVII, XXVIII, XXIX, XXX, XXXI, XXXII, XXXIII, XXXIV, XXXV, or XXXVI, wherein L¹ is a covalent bond, and R² is —R^{2A}.

Examples of compounds of the present disclosure include those listed in the Tables and exemplification herein, or a pharmaceutically acceptable salt, stereoisomer, or mixture of stereoisomers thereof. In some embodiments, the present disclosure provides a compound selected from those depicted in Table 1, below, or a pharmaceutically acceptable salt, stereoisomer, or mixture of stereoisomers thereof. In some embodiments, the present disclosure provides a compound set forth in Table 1, below, or a pharmaceutically acceptable salt thereof. In some embodiments, the present disclosure provides a compound set forth in Table 1, below.

Lengthy table referenced here
US20250313524A1-20251009-T00001
Please refer to the end of the specification for access instructions.

In chemical structures in Table 1, above, and the Examples, below, stereogenic centers are described according to the Enhanced Stereo Representation format (MDL/Biovia, e.g., using labels “or1”, “or2”, “abs”, “and1”). (See, for example, the structures of Compounds 1-21, 1-23, 1-29, 1-30, etc.)

In some embodiments, the present disclosure provides a compound in Table 1, above, wherein the compound is denoted as having an ADP-Glo IC₅₀ of “A”. In some embodiments, the present disclosure provides a compound in Table 1, above, wherein the compound is denoted as having an ADP-Glo IC₅₀ of “A” or “B”. In some embodiments, the present disclosure provides a compound in Table 1, above, wherein the compound is denoted as having an ADP-Glo IC₅₀ of “A” or “B” or “C”. In some embodiments, the present disclosure provides a compound in Table 1, above, wherein the compound is denoted as having an ADP-Glo IC₅₀ of “A” or “B” or “C” or “D”.

In some embodiments, the present disclosure provides a compound in Table 1, above, wherein the compound is denoted as having an MCF10A IC₅₀ of “A”. In some embodiments, the present disclosure provides a compound in Table 1, above, wherein the compound is denoted as having an MCF10A IC₅₀ of “A” or “B”. In some embodiments, the present disclosure provides a compound in Table 1, above, wherein the compound is denoted as having an MCF10A IC₅₀ of “A” or “B” or “C”. In some embodiments, the present disclosure provides a compound in Table 1, above, wherein the compound is denoted as having an MCF10A IC₅₀ of “A” or “B” or “C” or “D”.

In some embodiments, the present disclosure comprises a compound of formula I selected from those depicted in Table 1, above, or a pharmaceutically acceptable salt, stereoisomer, or mixture of stereoisomers thereof. In some embodiments, the present disclosure provides a compound of formula I selected from those depicted in Table 1, above, or a pharmaceutically acceptable salt thereof. In some embodiments, the present disclosure provides a compound of formula I selected from those depicted in Table 1, above.

In some embodiments, the present disclosure comprises a compound of formula II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, or XXXVI selected from those depicted in Table 1, above, or a pharmaceutically acceptable salt, stereoisomer, or mixture of stereoisomers thereof. In some embodiments, the present disclosure provides a compound of formula II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, or XXXVI selected from those depicted in Table 1, above, or a pharmaceutically acceptable salt thereof. In some embodiments, the present disclosure provides a compound of formula II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, or XXXVI selected from those depicted in Table 1, above.

### 4. Uses, Formulation, and Administration

### Pharmaceutically Acceptable Compositions

According to another embodiment, the disclosure provides a composition comprising a compound of this disclosure, or a pharmaceutically acceptable derivative thereof, and a pharmaceutically acceptable carrier, adjuvant, or vehicle. In some embodiments, the disclosure provides a pharmaceutical composition comprising a compound of this disclosure, and a pharmaceutically acceptable carrier. The amount of compound in compositions of this disclosure is such that it is effective to measurably inhibit a PI3Kα protein kinase, or a mutant thereof, in a biological sample or in a patient. In certain embodiments, the amount of compound in compositions of this disclosure is such that it is effective to measurably inhibit a PI3Kα protein kinase, or a mutant thereof, in a biological sample or in a patient. In certain embodiments, a composition of this disclosure is formulated for administration to a patient in need of such composition. In some embodiments, a composition of this disclosure is formulated for oral administration to a patient.

The terms “subject” and “patient,” as used herein, mean an animal (i.e., a member of the kingdom animal), preferably a mammal, and most preferably a human. In some embodiments, the subject is a human, mouse, rat, cat, monkey, dog, horse, or pig. In some embodiments, the subject is a human. In some embodiments, the subject is a mouse, rat, cat, monkey, dog, horse, or pig.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily active metabolite or residue thereof.

As used herein, the term “inhibitorily active metabolite or residue thereof” means that a metabolite or residue thereof is also an inhibitor of a PI3Kα protein kinase, or a mutant thereof.

Compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this disclosure may be administered in the form of suppositories for rectal or vaginal administration. These can be prepared by mixing the agent with a suitable non-irritating excipient that is solid at room temperature but liquid at rectal or vaginal temperature and therefore will melt in the rectum or vagina to release the drug. Such materials include cocoa butter, beeswax and polyethylene glycols.

Pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Preferably, pharmaceutically acceptable compositions of this disclosure are formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

The amount of compounds of the present disclosure that may be combined with the carrier materials to produce a composition in a single dosage form will vary depending upon the patient treated and the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

The precise dose to be employed in the compositions will also depend on the route of administration and should be decided according to the judgment of the practitioner and each subject's circumstances. In specific embodiments of the disclosure, suitable dose ranges for oral administration of the compounds of the disclosure are generally about 1 mg/day to about 1000 mg/day. In some embodiments, the oral dose is about 1 mg/day to about 800 mg/day. In some embodiments, the oral dose is about 1 mg/day to about 500 mg/day. In some embodiments, the oral dose is about 1 mg/day to about 250 mg/day. In some embodiments, the oral dose is about 1 mg/day to about 100 mg/day. In some embodiments, the oral dose is about 5 mg/day to about 50 mg/day. In some embodiments, the oral dose is about 5 mg/day. In some embodiments, the oral dose is about 10 mg/day. In some embodiments, the oral dose is about 20 mg/day. In some embodiments, the oral dose is about 30 mg/day. In some embodiments, the oral dose is about 40 mg/day. In some embodiments, the oral dose is about 50 mg/day. In some embodiments, the oral dose is about 60 mg/day. In some embodiments, the oral dose is about 70 mg/day. In some embodiments, the oral dose is about 100 mg/day. It will be recognized that any of the dosages listed herein may constitute an upper or lower dosage range and may be combined with any other dosage to constitute a dosage range comprising an upper and lower limit.

In some embodiments, pharmaceutically acceptable compositions contain a provided compound and/or a pharmaceutically acceptable salt thereof at a concentration ranging from about 0.01 to about 90 wt %, about 0.01 to about 80 wt %, about 0.01 to about 70 wt %, about 0.01 to about 60 wt %, about 0.01 to about 50 wt %, about 0.01 to about 40 wt %, about 0.01 to about 30 wt %, about 0.01 to about 20 wt %, about 0.01 to about 2.0 wt %, about 0.01 to about 1 wt %, about 0.05 to about 0.5 wt %, about 1 to about 30 wt %, or about 1 to about 20 wt %. The composition can be formulated as a solution, suspension, ointment, or a capsule, and the like. The pharmaceutical composition can be prepared as an aqueous solution and can contain additional components, such as preservatives, buffers, tonicity agents, antioxidants, stabilizers, viscosity-modifying ingredients and the like.

Pharmaceutically acceptable carriers are well-known to those skilled in the art, and include, e.g., adjuvants, diluents, excipients, fillers, lubricants and vehicles. In some embodiments, the carrier is a diluent, adjuvant, excipient, or vehicle. In some embodiments, the carrier is a diluent, adjuvant, or excipient. In some embodiments, the carrier is a diluent or adjuvant. In some embodiments, the carrier is an excipient.

Examples of pharmaceutically acceptable carriers may include, e.g., water or saline solution, polymers such as polyethylene glycol, carbohydrates and derivatives thereof, oils, fatty acids, or alcohols. Non-limiting examples of oils as pharmaceutical carriers include oils of petroleum, animal, vegetable or synthetic origin, such as peanut oil, soybean oil, mineral oil, sesame oil and the like. The pharmaceutical carriers may also be saline, gum acacia, gelatin, starch paste, talc, keratin, colloidal silica, urea, and the like. In addition, auxiliary, stabilizing, thickening, lubricating and coloring agents may be used. Other examples of suitable pharmaceutical carriers are described in e.g., Remington's: The Science and Practice of Pharmacy, 22nd Ed. (Allen, Loyd V., Jr ed., Pharmaceutical Press (2012)); Modern Pharmaceutics, 5^{th} Ed. (Ale^{YA}nder T. Florence, Juergen Siepmann, CRC Press (2009)); Handbook of Pharmaceutical Excipients, 7^{th} Ed. (Rowe, Raymond C.; Sheskey, Paul J.; Cook, Walter G.; Fenton, Marian E. eds., Pharmaceutical Press (2012)) (each of which is hereby incorporated by reference in its entirety).

The pharmaceutically acceptable carriers employed herein may be selected from various organic or inorganic materials that are used as materials for pharmaceutical formulations and which are incorporated as analgesic agents, buffers, binders, disintegrants, diluents, emulsifiers, excipients, extenders, glidants, solubilizers, stabilizers, suspending agents, tonicity agents, vehicles and viscosity-increasing agents. Pharmaceutical additives, such as antioxidants, aromatics, colorants, flavor-improving agents, preservatives, and sweeteners, may also be added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc and water, among others. In some embodiments, the term “pharmaceutically acceptable” means approved by a regulatory agency of the Federal or a state government or listed in the U.S. Pharmacopeia or other generally recognized pharmacopeia for use in animals, and more particularly in humans.

Surfactants such as, e.g., detergents, are also suitable for use in the formulations. Specific examples of surfactants include polyvinylpyrrolidone, polyvinyl alcohols, copolymers of vinyl acetate and of vinylpyrrolidone, polyethylene glycols, benzyl alcohol, mannitol, glycerol, sorbitol or polyoxyethylenated esters of sorbitan; lecithin or sodium carboxymethylcellulose; or acrylic derivatives, such as methacrylates and others, anionic surfactants, such as alkaline stearates, in particular sodium, potassium or ammonium stearate; calcium stearate or triethanolamine stearate; alkyl sulfates, in particular sodium lauryl sulfate and sodium cetyl sulfate; sodium dodecylbenzenesulphonate or sodium dioctyl sulphosuccinate; or fatty acids, in particular those derived from coconut oil, cationic surfactants, such as water-soluble quaternary ammonium salts of formula N⁺R′R″R′″R″″Y⁻, in which the R radicals are identical or different optionally hydroxylated hydrocarbon radicals and Y⁻ is an anion of a strong acid, such as halide, sulfate and sulfonate anions; cationic surfactants, such as cetyltrimethylammonium bromide; amine salts of formula N⁺R′R″R′″, in which the R radicals are identical or different optionally hydroxylated hydrocarbon radicals; cationic surfactants, such as octadecylamine hydrochloride; non-ionic surfactants, such as optionally polyoxyethylenated esters of sorbitan, in particular Polysorbate 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide; and amphoteric surfactants, such as substituted lauryl compounds of betaine.

Suitable pharmaceutical carriers may also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, polyethylene glycol 300, water, ethanol, polysorbate 20, and the like. The present compositions, if desired, may also contain wetting or emulsifying agents, or pH buffering agents.

Tablets and capsule formulations may further contain one or more adjuvants, binders, diluents, disintegrants, excipients, fillers, or lubricants, each of which are known in the art. Examples of such include carbohydrates such as lactose or sucrose, dibasic calcium phosphate anhydrous, corn starch, mannitol, xylitol, cellulose or derivatives thereof, microcrystalline cellulose, gelatin, stearates, silicon dioxide, talc, sodium starch glycolate, acacia, flavoring agents, preservatives, buffering agents, disintegrants, and colorants. Orally administered compositions may contain one or more optional agents such as, e.g., sweetening agents such as fructose, aspartame or saccharin; flavoring agents such as peppermint, oil of wintergreen, or cherry; coloring agents; and preservative agents, to provide a pharmaceutically palatable preparation.

### Uses of Compounds and Pharmaceutically Acceptable Compositions

Compounds and compositions described herein are generally useful for the inhibition of a kinase or a mutant thereof. In some embodiments, the kinase inhibited by the compounds and compositions described herein is a phosphatidylinositol 3-kinase (PI3K). In some embodiments, the kinase inhibited by the compounds and compositions described herein is one or more of a PI3Kα, PI3Kδ, and PI3Kγ. In some embodiments, the kinase inhibited by the compounds and compositions described herein is a PI3Kα. In some embodiments, the kinase inhibited by the compounds and compositions described herein is a PI3Kα containing at least one of the following mutations: H1047R, E542K, and E545K.

Compounds or compositions of the disclosure can be useful in applications that benefit from inhibition of PI3K enzymes. For example, PI3K inhibitors of the present disclosure are useful for the treatment of cellular proliferative diseases generally. Compounds or compositions of the disclosure can be useful in applications that benefit from inhibition of PI3Kα enzymes. For example, PI3Kα inhibitors of the present disclosure are useful for the treatment of cellular proliferative diseases generally.

Aberrant regulation of PI3K, which often increases survival through Aid activation, is one of the most prevalent events in human cancer and has been shown to occur at multiple levels. The tumor suppressor gene PTEN, which dephosphorylates phosphoinositides at the 3′ position of the inositol ring, and in so doing antagonizes PI3K activity, is functionally deleted in a variety of tumors. In other tumors, the genes for the p110 alpha isoform, PIK3CA, and for Akt are amplified, and increased protein expression of their gene products has been demonstrated in several human cancers. Furthermore, mutations and translocation of p85 alpha that serve to up-regulate the p85-p110 complex have been described in human cancers. Finally, somatic missense mutations in PIK3CA that activate downstream signaling pathways have been described at significant frequencies in a wide diversity of human cancers (Kang et el., Proc. Natl. Acad. Sci. USA 102:802 (2005); Samuels et al., Science 304:554 (2004); Samuels et al., Cancer Cell 7:561-573 (2005)). These observations show that deregulation of phosphoinositol-3 kinase, and the upstream and downstream components of this signaling pathway, is one of the most common deregulations associated with human cancers and proliferative diseases (Parsons et al., Nature 436:792 (2005); Hennessey at el., Nature Rev. Drug Disc. 4:988-1004 (2005)).

The activity of a compound utilized in this disclosure as an inhibitor of a PI3K kinase, for example, a PI3Kα, or a mutant thereof, may be assayed in vitro, in vivo or in a cell line. In vitro assays include assays that determine inhibition of either the phosphorylation activity and/or the subsequent functional consequences, or ATPase activity of an activated PI3Kα, or a mutant thereof. Alternative in vitro assays quantitate the ability of the inhibitor to bind to a a PI3Kα. Inhibitor binding may be measured by radiolabeling the inhibitor prior to binding, isolating the inhibitor/PI3Kα complex and determining the amount of radiolabel bound. Alternatively, inhibitor binding may be determined by running a competition experiment where new inhibitors are incubated with a PI3Kα bound to known radioligands. Representative in vitro and in vivo assays useful in assaying a PI3Kα inhibitor include those described and disclosed in the patent and scientific publications described herein. Detailed conditions for assaying a compound utilized in this disclosure as an inhibitor of a PI3Kα, or a mutant thereof, are set forth in the Examples below.

### Treatment of Disorders

Provided compounds are inhibitors of PI3Kα and are therefore useful for treating one or more disorders associated with activity of PI3Kα or mutants thereof. Thus, in certain embodiments, the present disclosure provides a method of treating a PI3Kα-mediated disorder in a subject, comprising administering a therapeutically effective amount of a compound of the present disclosure, or a pharmaceutically acceptable salt thereof, or a pharmaceutically acceptable composition of either of the foregoing, to a subject in need thereof. In certain embodiments, the present disclosure provides a method of treating a PI3Kα-mediated disorder in a subject comprising administering a therapeutically effective amount of a compound of the present disclosure, or a pharmaceutically acceptable composition thereof, to a subject in need thereof. In some embodiments, the subject has a mutant PI3Kα. In some embodiments, the subject has PI3Kα containing at least one of the following mutations: H1047R, E542K, and E545K. In some embodiments, the subject has PI3Kα containing at least one of the mutations in Table A:

TABLE A

| | | | |
|---|---|---|---|
| | R4G | G8C | E9_M16 > V |
| M1* | R4L | G8D | E9_M16del |
| M1I | R4P | G8R | E9_P17 > A |
| M1L | R4Q | G8S | E9_P17del |
| M1R | R4_P17del | G8V | E9_P18 > CPT |
| M1T | R4_P18del | G8_P17del | E9_P18 > GCPT |
| M1V | P5T | G8_P18del | E9_P18del |
| P2H | P5_R19del | G8_R19del | E9_R19 > G |
| P2L | S6L | E9* | E9_R19del |
| P2S | S6_I13del | E9A | E9fs*6 |
| P3A | S7* | E9G | L10P |
| P3L | S7L | E9K | L10Q |
| P3S | S7_P18del | E9Q | L10R |
| P3T | S7_R19del | E9_I20 > V | L10V |
| R4* | S7fs*3 | E9_L15 > G | L10_L15 > R |
| L10_L15del | L15W | V32L | E52* |
| L10_L21del | M16I | V32M | E52K |
| L10_M16del | M16K | T33A | E52Q |
| L10_P17 > T | M16L | T33I | A53S |
| L10_P17del | M16R | T33S | A53V |
| L10_P18 > Q | M16T | E35* | A53fs*19 |
| L10_P18del | M16V | E35K | R54I |
| W11* | P17A | E35Q | R54K |
| W11C | P17L | C36* | K55I |
| W11G | P17R | L37F | K55Q |
| W11L | P17S | L37I | Y56* |
| W11R | P18L | R38C | Y56H |
| W11S | P18Q | R38G | P57H |
| W11_H14del | P18S | R38H | P57L |
| W11_L15del | P18T | R38L | P57S |
| W11_L21del | P18fs*4 | R38S | L58F |
| W11_M16del | R19* | R38_I43 > L | L58R |
| W11_P17 > S | R19I | E39* | L58V |
| W11_P18 > S | R19K | E39A | L58fs*13 |
| W11_P18 > CG | I20M | E39D | L58fs*14 |
| W11_P18del | I20T | E39K | H59L |
| W11_R19del | I20fs*3 | E39Q | H59N |
| W11_V22del | L21I | A40V | H59R |
| W11del | L21R | T41I | H59Y |
| G12C | L21V | L42* | Q60* |
| G12D | V22G | L42F | Q60E |
| G12S | V22I | I43L | Q60L |
| G12V | V22L | I43M | Q60P |
| G12_L21del | E23K | T44F | L61F |
| G12_P17 > A | E23Q | T44I | L61I |
| G12_P18 > I | C24F | I45L | L61R |
| I13F | C24Y | I45T | L62F |
| I13M | L25* | I45V | L62I |
| I13N | L25F | K46M | L62del |
| I13V | L25S | H47L | Q63* |
| I13_P18del | P27S | H47Q | Q63E |
| I13del | G29K | H47Y | Q63H |
| H14D | G29R | E48* | Q63L |
| H14N | M30I | E48K | Q63fs*9 |
| H14Y | M30V | E48Q | D64G |
| H14_I20del | M30fs*9 | E48V | D64H |
| H14_L21del | I31M | F50L | D64N |
| L15S | I31V | F50fs*22 | D64V |
| L15V | V32A | K51R | D64Y |
| D64_S72 > VL | E78G | R88L | V101I |
| E65* | E78K | R88P | V101L |
| E65A | E78Q | R88Q | V101del |
| E65G | E78_R79insVSK | L89F | I102F |
| E65K | NTYLSKCYS | L89H | I102M |
| E65V | R79M | C90F | I102T |
| S66C | R79W | C90G | I102V |
| S66F | R79_E80 > K | C90S | I102_E103 > K |
| S66Y | E80K | C90W | I102_E103ins16 |
| S67A | E80_E81 > KK | C90Y | I102_P104 > K |
| S67C | E81* | C90fs*1 | I102_P104 > T |
| S67F | E81A | L92F | I102_P104del |
| S67Y | E81D | L92I | I102_V105del |
| S67del | E81G | R93G | I102del |
| Y68C | E81K | R93L | I102fs*6 |
| Y68H | E81Q | R93L | E103* |
| I69F | E81V | R93P | E103D |
| I69L | E81_F82 > V | R93Q | E103G |
| I69N | E81del | R93W | E103K |
| I69S | F82I | L94F | E103Q |
| I69V | F82L | F95C | E103_E110del |
| F70V | F83C | F95S | E103_G106 > D |
| V71G | F83I | F95fs*2 | E103_N107del |
| V71I | F83K | Q96* | E103_P104 > A |
| V71L | F83L | Q96E | E103_P104 > S |
| S72G | F83S | Q96H | E103_P104del |
| S72R | F83V | Q96K | E103_R108 > YC |
| S72T | F83Y | Q96R | E103_V105 > A |
| S72fs*27 | F83del | Q96fs*4 | E103_V105 > D |
| V73A | F83fs*17 | P97F | E103del |
| V73I | D84H | P97H | P104A |
| T74A | D84N | P97L | P104K |
| T74I | D84Y | P97S | P104L |
| T74S | D84fs*1 | F98C | P104Q |
| Q75* | E85* | F98L | P104R |
| Q75E | E85K | F98V | P104S |
| Q75H | E85Q | L99* | P104T |
| Q75L | T86I | L99F | P104_E110 > Q |
| Q75P | T86S | L99V | P104_E110del |
| E76Q | R87G | L99fs*1 | P104_G106 > R |
| E76del | R87I | K100* | P104_G106 > S |
| A77G | R87S | K100N | P104_G106del |
| A77P | R87T | K100del | P104_N107 > H |
| E78* | R88* | V101A | P104_N107del |
| | R88G | | |
| P104_V105 > I | G106del | E109fs*11 | I112_L113insQI |
| P104_V105 > L | N107H | E110* | I112 > MSM |
| P104_V105del | N107I | E110K | I112del |
| P104_V105insV | N107K | E110Q | L113F |
| GNREEKILNREIG | N107S | E110_E116del | L113H |
| MIQY | N107T | E110_I112 > D | L113I |
| P104del | N107Y | E110_I112del | L113R |
| V105A | N107_E109 > K | E110_K111 > D | L113V |
| V105I | N107_E109del | E110_K111 > G | L113_I117del |
| V105L | N107_E109del | E110_K111 > M | L113_N114 > H |
| V105_E109 > A | N107_E110del | E110_K111 > V | L113_N114del |
| V105_E109 > K | N107_K111 > YRE | E110_K111del | L113_N114insL |
| V105_E109 > EE | N107_R108 > S | E110_K111insE | L113del |
| V105_E109del | N107_R108del | E110_N114 > D | N114D |
| V105_E110del | N107del | E110_R115 > G | N114H |
| V105_G106del | R108C | E110del | N114I |
| V105_N107 > D | R108G | K111E | N114S |
| V105_N107 > Y | R108H | K111M | N114Y |
| V105_N107 > AT | R108L | K111N | N114_1117del |
| V105_N107 > GD | R108P | K111Q | N114_R115 > K |
| V105_N107del | R108S | K111R | N114_R115insL |
| V105_N107del | R108_E109 > C | K111T | N |
| V105_R108del | R108_E109 > Q | K111_E116del | N114del |
| V105del | R108_E109del | K111_I112 > D | R115* |
| V105del | R108_E109insLK | K111_I112 > F | R115E |
| G106A | VIEPVGNR | K111_I112 > N | R115G |
| G106C | R108_E110del | K111_I112del | R115L |
| G106D | R108_I112 > L | K111_I112insK | R115P |
| G106E | R108_I112 > V | K111_I112insEK | R115Q |
| G106F | R108_I112del | K111_L113 > I | R115_E116insD |
| G106L | R108_K111 > Q | K111_L113del | EEKILNR |
| G106R | R108_K111 > EA | K111_N114 > D | R115del |
| G106S | R108_K111del | K111_N114del | E116* |
| G106V | R108del | K111_R115del | E116G |
| G106_E109 > A | E109* | K111del | E116K |
| G106_E109 > E | E109A | I112D | E116Q |
| G106_E109del | E109G | I112F | E116_I117insRE |
| G106_E110 > K | E109K | I112L | I117F |
| G106_I112 > F | E109_E110 > A | I112N | I117V |
| G106_K111 > E | E109_I112 > D | I112S | I117_G118insEI |
| G106_N107 > T | E109_I112 > V | I112T | G118D |
| G106_N107del | E109_L113 > D | I112V | G118E |
| G106_R108 > I | E109_L113 > V | I112_L113del | G118V |
| G106_R108 > V | E109_N114del | I112_L113insl | |
| G106_R108del | E109_R115 > G | | |
| G118_F119insM | M130T | C147S | R162K |
| IEPVGNREEKILN | V131F | C147Y | R162T |
| REIG | V131I | K148E | A163G |
| G118_F119insM | K132T | E149* | A163T |
| IQEILNREIG | D133G | E149A | A163V |
| G118_F119insM | D133H | E149D | M164I |
| IQYPQSILNREIG | D133Y | E149G | M164L |
| F119I | D133fs*12 | E149K | M164T |
| F119L | P134L | E149Q | M164V |
| A120S | P134S | E149V | Y165C |
| A120T | E135* | A150G | Y165F |
| I121L | E135K | A150S | YV165_166*F |
| I121N | E135Q | A150V | V166A |
| G122A | E135fs*3 | V151A | V166G |
| G122C | Q137* | V151L | V166I |
| G122D | Q137H | V151M | Y167* |
| G122S | Q137L | D152H | Y167C |
| G122V | D138G | D152N | Y167H |
| M123I | D138N | L153F | P168F |
| M123K | D138Y | R154K | P168H |
| M123L | F139I | R154M | P168L |
| M123V | F139L | R154S | P168R |
| P124A | F139S | R154W | P168S |
| P124L | F139Y | D155H | P168T |
| P124Q | F139_R140 > S* | D155V | P169A |
| P124S | R140* | D155Y | P169L |
| P124_V125 > Q | R140Q | L156I | P169Q |
| V125A | R141I | L156V | P169R |
| V125E | R141K | N157K | P169S |
| V125G | R141T | S158* | P169T |
| V125L | R141fs*4 | S158A | N170fs*2 |
| V125M | N142D | S158L | V171E |
| C126S | I143T | S158P | V171L |
| C126W | L144P | P159S | E172D |
| C126Y | L144V | P159T | E172G |
| C126fs*19 | N145K | H160N | E172Q |
| E127* | N145S | H160R | S173C |
| E127D | N145T | H160Y | S173Y |
| E127K | N145Y | H160fs*12 | S174L |
| E127Q | N145_V146 > KI | S161C | P175L |
| F128Y | V146A | S161I | P175Q |
| D129H | V146G | S161T | P175S |
| D129N | V146I | R162G | E175D |
| D129Y | C147F | R162I | E175K |
| M130I | | | |
| E176Q | I197T | V216I | M232V |
| L177* | I197V | V216L | L233F |
| L177F | V198D | P217L | L233S |
| P178S | V198F | P217R | L233V |
| K179N | V198I | P217S | L234fs*8 |
| K179Q | S199F | E218D | S235C |
| H180N | S199P | E218K | S235F |
| H180P | S199Y | E218Q | S235P |
| H180R | P200A | E218fs*4 | S235T |
| I181V | P200L | Q219H | S236C |
| Y182F | P200R | Q219K | S236F |
| Y182H | P200S | V220L | S236T |
| N183H | N201D | I221N | S236Y |
| N183T | N201S | I221V | E237K |
| K184E | N202D | E223D | E237Q |
| K184N | N202fs*9 | E223G | Q238K |
| K184Q | D203G | E223K | Q238L |
| K184R | D203H | E223Q | L239I |
| K184T | D203N | A224G | L239Q |
| L185* | D203Y | A224S | L239R |
| L185F | K204T | A224T | L239V |
| L185S | Q205* | A224V | K240N |
| D186A | Q205H | I225M | K240Q |
| D186E | Q205K | I225S | K240T |
| D186G | Q205P | I225T | V243D |
| D186N | Q205R | I225V | V243G |
| D186Y | K206E | R226G | V243I |
| K187E | Y207S | R226S | V243L |
| K187Q | T208A | R226T | V243fs*15 |
| K187R | L209V | K227* | E245K |
| G188R | K210R | K228N | Y246C |
| Q189* | I211M | T229A | Q247* |
| Q189fs*14 | I211V | T229P | Q247H |
| I190L | N212S | T229S | Q247L |
| I190V | H213P | T229fs*9 | Q247R |
| V192L | H213Y | T229fs*11 | G248C |
| V192M | D214E | R230* | G248V |
| V193L | D214G | R230G | K249* |
| I194M | D214Y | R230L | K249N |
| W195* | D214_C215 > ER | R230Q | K249T |
| W195C | C215S | R230fs*7 | Y250C |
| W195R | C215T | S231R | Y250F |
| V196L | C215Y | M232I | Y250H |
| I197M | V216A | M232L | Y250N |
| I251N | S268N | G280R | P298Q |
| K253I | S268T | G280V | P298R |
| K253N | Q269* | G280W | P298S |
| K253T | Q269H | R281M | M299I |
| V254G | Q269L | R281S | M299L |
| V254L | Q269R | R281fs*5 | M299T |
| V254M | Q269fs*4 | M282I | D300A |
| V254_C255 > LW | Y270F | P283L | D300E |
| C255F | Y270N | P283S | D300H |
| C255Y | K271N | N284H | D300N |
| C255fs*3 | K271R | N284K | C301* |
| G256* | Y272C | N284S | C301F |
| G256A | Y272F | N284Y | C301G |
| G256E | Y272H | L285F | C301S |
| G256R | I273L | L285M | C301W |
| G256V | I273M | M286I | C301Y |
| C257R | I273T | L287F | F302C |
| D258E | R274G | M288I | F302Y |
| D258G | R274I | M288T | T303A |
| D258H | R274K | A289T | T303K |
| D258N | R274T | A289V | T303R |
| E259Q | S275C | K290N | M304I |
| Y260C | S275I | E291* | M304L |
| Y260H | S275N | E291K | M304T |
| F261L | C276* | E291Q | M304V |
| F261V | C276F | S292C | P305S |
| F261Y | C276G | S292I | P305_N319 > H |
| E263* | I277L | S292N | S306A |
| E263D | I277T | S292R | S306C |
| E263K | I277V | L293F | S306F |
| E263Q | I277fs*7 | Y294* | S306P |
| E263fs*5 | M278I | Y294H | S306Y |
| K264Q | M278K | Y294fs*25 | S306fs*13 |
| K264T | M278L | S295A | Y307F |
| K264fs*4 | M278T | S295C | Y307H |
| Y265* | M278fs*23 | S295F | S308A |
| Y265C | L279F | S295Y | S308C |
| P266H | L279H | Q296* | S308F |
| P266L | L279I | Q296E | S308Y |
| P266R | L279R | Q296K | R309G |
| P266S | L279V | Q296R | R309_R310 > S |
| L267M | G280A | L297P | R310C |
| L267P | G280E | L297R | R310H |
| L267V | G280K | P298A | R310L |
| I311F | S326P | C340F | V344_N345insK |
| I311N | S326Y | C340R | V |
| S312C | L327F | C340_A341insV | V344_N345insKI |
| S312F | L327H | KILC | LCATYV |
| S312fs*18 | L327I | A341S | V344_N345insT |
| T313I | L327fs*4 | A341V | TYV |
| T313K | W328* | A341_T342insIKI | V344_N345insT |
| T315I | W328C | LCA | YV |
| P316L | W328L | A341_T342insLR | V344_N347del |
| P316Q | W328R | IKILCA | N345D |
| P316S | W328S | A341_T342insYK | N345H |
| P316T | V329F | ILCA | N345I |
| Y317C | V329G | T342A | N345K |
| Y317F | V329I | T342I | N345S |
| Y317H | I330L | T342S | N345T |
| M318I | I330V | T342_N345 > H | N345Y |
| M318T | N331H | T342_Y343ins37 | N345_I348 > K |
| M318V | S332I | T342_Y343insRI | N345_I348del |
| M318fs*15 | S332T | KILCAT | N345_K353del |
| N319S | A333S | Y343C | N345_V346 > K |
| N319T | A333T | Y343F | N345_V346 > KL |
| G320* | A333V | Y343I | N345_V346 > KA |
| G320A | L334F | Y343L | TYVNV |
| G320E | R335I | Y343S | N345_V346insA |
| G320R | R335K | Y343_N345del | TYVN |
| G320V | R335S | Y343_V344 > L | V346A |
| E321A | R335T | Y343_V344insA | V346E |
| E321K | R335_I336ins18 | TY | V346G |
| E321Q | R335fs*2 | Y343_V344insER | V346L |
| E321V | R335fs*17 | IKILCATY | V346L |
| S323A | R335fs*33 | Y343_V344insLC | V346Q |
| S323C | I336M | ATY | V346_N347 > ERT |
| S323F | I336fs*8 | V344A | YVNVN |
| S323P | K337Q | V344E | V346_N347insK |
| S323Y | K337T | V344F | V346_N347insV |
| T324I | I338F | V344G | V346_N347insE |
| K325E | I338N | V344L | KIKKKKKK |
| K325Q | I338S | V344M | V346_N347insK |
| K325R | I338T | V344R | NV |
| K325_I330del | I338fs*7 | V344_N345 > RFS | V346_N347insM |
| K325fs*6 | L339F | AFWLRSS | NV |
| S326A | L339I | V344_N345insK | V346_N347insV |
| S326C | L339R | V344_N345insM | NV |
| S326F | L339V | V344_N345insV | V346 > GK |
| | | V344_N345insIL | N347D |
| | | CATYV | N347I |
| | | | N347K |
| N347T | I354S | E365K | R382fs*6 |
| N347Y | I354T | E365Q | W383* |
| N347_I348insR | I354V | E365V | W383C |
| N347_I348insVN | Y355C | P366F | W383L |
| I348M | Y355_V356insY | P366H | E385K |
| I348S | V356A | P366L | W386R |
| I348V | V356F | P366R | L387Q |
| I348_R349insLNI | V356I | P366S | L387V |
| R349* | V356L | P366T | N388D |
| R349Q | R357* | P366fs*5 | N388I |
| R349_D350insIR | R357G | C368Y | N388T |
| R349_D350insV | R357L | D369G | Y389C |
| R | R357Q | D369N | Y389F |
| D350G | R357fs*10 | D369Y | Y389S |
| D350H | T358A | N370D | D390A |
| D350K | T358K | N370K | D390H |
| D350N | T358S | N370S | D390N |
| D350Y | T358_G359insA | N372S | D390Y |
| D350_I351insKK | G359A | T373I | I391V |
| ILCATYVNVNIRD | G359C | T373P | I391fs*36 |
| D350_I351insRI | G359R | T373S | Y392* |
| KILCATYVNVNIRD | G359V | Q374* | Y392H |
| D350del | I360F | Q374E | P394S |
| I351F | I360T | Q374H | P394_D395ins2 |
| I351S | I360V | R375G | 3 |
| I351_D352 > E | Y361C | R375I | D395H |
| I351_D352 > KYL | Y361F | R375K | D395N |
| Q | Y361H | R375S | D395V |
| I351_D352insGI | Y361_H362insQI | V376I | D395Y |
| KILCATYVNVNIR | YVRTGIY | P377L | L396F |
| DI | H362N | P377S | L396I |
| I351_D352insVN | H362R | C378F | L396P |
| VNIRDI | H362Y | C378L | L396V |
| D352H | G363A | C378R | P397A |
| D352N | G363E | C378W | P397R |
| D352Y | G363V | C378Y | P397S |
| D352 > RDIN | G363_G364insY | S379C | P397T |
| K353M | VRTGIYHG | S379F | R398C |
| K353N | G363 > YHR | N380K | R398H |
| K353_I354insVV | G364E | N380Y | R398L |
| NVNIRDIDK | G364K | P381A | A399D |
| I354D | G364R | P381S | A399G |
| I354F | G364_E365insV | R382K | A399S |
| I354L | RTGIYHGG | R382W | A399T |
| I354N | E365D | | A399V |
| R401* | K416E | P421S | T433A |
| R401L | K416I | P421T | T433R |
| R401Q | E417D | P421_A423 > H | T433_D434 > NTD |
| R401S | E417G | P421_A423 > H | T433_D434insTL |
| L402V | E417K | P421_A423del | VSGKMALNLWP |
| C403R | E417Q | P421_L422del | VPHGLE |
| L404F | E417V | P421 > RR | D434E |
| L404I | E418* | L422E | D434H |
| L404V | E418A | L422F | D434fs*2 |
| S405F | E418K | L422S | T435I |
| S405T | E418Q | L422W | T435N |
| S405Y | E418_C420 > D | L422_A423 > F | T435S |
| I406F | E418_P421 > A | A423E | L436P |
| I406M | H419L | A423S | L436V |
| I406V | H419P | A423T | L436fs*1 |
| C407F | H419Q | A423V | L436fs*32 |
| C407R | H419R | W424* | V437E |
| C407W | H419Y | W424C | V437G |
| C407Y | H419_C420 > R | W424G | V437I |
| C407fs*21 | H419_C420del | W424L | S438A |
| S408C | H419_L422 > T | W424R | S438C |
| S408P | H419_L422 > LM | W424_G425insF | S438fs*30 |
| V409F | H419_L422 > PW | W424_I427del | G439A |
| V409I | H419_L422del | G425E | G439E |
| K410_G411insG | H419_L422del | G425R | G439K |
| RKGAKEVKYFRR | H419_P421 > L | G425V | G439R |
| K | H419_P421 > P | N426D | G439fs*5 |
| K410fs*6 | H419_P421 > Q | N426S | K440E |
| G411D | H419_P421 > R | N426fs*6 | K440N |
| G411R | H419_P421 > T | I427K | K440fs*45 |
| G411S | H419_P421del | I427M | M441I |
| G411V | H419fs*11 | I427T | M441V |
| R412* | C420R | I427V | E441fs*3 |
| R412L | C420S | N428K | M441fs*3 |
| R412Q | C420Y | N428S | M441fs*28 |
| K413N | C420_P421del | N428Y | A442T |
| K413_G414insRK | C420_L422 > W | L429F | A442V |
| G414A | C420_A423 > W | L429V | L443F |
| G414D | C420_A423 > Y | L429fs*2 | N444H |
| G414R | C420_A423del | F430C | N444K |
| G414S | C420_I427 > WH | F430L | N444_G451 > K |
| G414V | GNV | Y432F | N444_L455 > H |
| G414fs*13 | P421A | Y432H | L445F |
| A415D | P421L | Y432fs*5 | L445I |
| | P421R | | |
| L445_W446insL | P449S | G451K | E453* |
| W446* | P449T | G451R | E453A |
| W446S | P449_D454 > R | G451V | E453D |
| W446_D454del | P449_D454del | G451_D454 > RR | E453G |
| W446_E453del | P449_E453 > Q | G451_D454del | E453K |
| W446_G451del | P449_E453del | G451_E453del | E453Q |
| W446_G460 > C | P449_H450insL | G451_G460del | E453V |
| W446_H450 > R | VSGKMALNLWP | G451_I459 > A | E453_D454 > KN |
| W446_H450del | VP | G451_I459 > V | E453_D454del |
| W446_I459del | P449_H450insP | G451_L452 > KKK | E453_D454insG |
| W446_L456del | VP | KK | KMALNLWPVPHGLE |
| W446_P447insW | P449_H450insP | G451_L452insF | E453_D454insV |
| W446_P458del | VP | GKMALNLWPVP | SGKMALNLWPVPHGLE |
| W446_V461del | P449_I459del | HG | E453_D454insV |
| P447A | P449_L452del | G451_L455 > A | VSGKMALNLWP |
| P447L | P449_L455del | G451_L455 > V | VPHGLE |
| P447S | P449_L456del | G451_L455 > GTM | E453_G460 > C |
| P447_L452del | P449_P458del | G451_L455del | E453_G460del |
| P447_L455del | H450D | G451_L456 > K | E453_G463del |
| P447_V448ins24 | H450N | G451_L456 > V | E453_I459 > G |
| P447_V448insLF | H450Q | G451_N457del | E453_I459 > V |
| DYTDTLVSGKMA | H450R | G451_P458 > V | E453_I459del |
| LNLWP | H450Y | G451_P458del | E453_L455 > G |
| V448A | H450_D454del | L452S | E453_L455 > V |
| V448E | H450_E453del | L452_E453del | E453_L455del |
| V448G | H450_G451 > PRG | L452_E453ins21 | E453_L455del |
| V448L | H450_G460 > R | L452_E453insA | E453_L456 > M |
| V448_D454del | H450_I459 > L | GKMALNLWPVPHGL | E453_L456 > V |
| V448_E453 > K | H450_I459del | L452_E453insVS | E453_L456del |
| V448_E453 > P | H450_L452del | GKMALNLWPVP | E453_P458del |
| V448_E453 > YK | H450_L455 > P | HGL | E453_T462del |
| V448_E453del | H450_L455 > Q | L452_G460 > F | E453_T462del |
| V448_G451del | H450_L455 > KM | L452_G460del | E453 > GLK |
| V448_L452del | H450_L455del | L452_I459 > FRRF | E453del |
| V448_L455del | H450_L455del | L452_I459 > PLW | D454E |
| V448_P449insS | H450_L455del | ARL | D454G |
| GKMALNLWPV | H450_L456 > P | L452_I459del | D454H |
| V448_P449insV | H450_L456del | L452_I459del | D454K |
| SGKMALNLWPV | H450_N457del | L452_L455 > W | D454N |
| V448fs*14 | H450_P458 > LIH | L452_N457 > T | D454Y |
| P449A | H450_P458del | L452_N457 > Y | D454_I459del |
| P449H | H450_V461 > GS | L452_P458 > F | D454_K468del |
| P449L | G451A | L452_T462 > QKT | D454_L455 > V |
| P449R | G451E | L452_V461 > F | D454_L455del |
| D454_N467 > VS | P458_V461 > L | K468_E469ins35 | E476_F477insLE |
| D454_P458 > Y | I459M | K468_E469insVE | D478A |
| D454del | I459N | RLLNPIGVTGSNP | D478E |
| L455F | I459S | NK | D478G |
| L455_G460 > C | I459T | K468_E469insVL | D478H |
| L455_G460del | I459V | LNPIGVTGSNPNK | D478N |
| L455_I459 > C | I459_T462del | E469* | D478Y |
| L455_I459 > C | G460A | E469A | W479C |
| L455_I459 > F | G460C | E469D | W479S |
| L455_I459del | G460D | E469G | F480L |
| L455_L456 > FM | G460R | E469K | S481G |
| L455_L456insPG | G460V | E469V | S481N |
| KMALNLWPVPHGLEDL | V461A | E469_T470 > D | S481R |
| L455_N467 > SD | V461_N465del | E469 > DK | S481T |
| L455_N467del | T462I | T470I | S482C |
| L455_P458del | T462_N465del | T470N | S482N |
| L455_T462del | T462_S464del | T470P | V483A |
| L455_V461 > F | T462fs*12 | T470S | V483L |
| L456M | G463* | T470fs*4 | V483M |
| L456P | G463A | P471A | V484A |
| L456R | G463E | P471I | V484I |
| L456V | G463R | P471L | V484L |
| L456_I459del | G463V | P471Q | K485E |
| L456_N457insKK | G463_K468del | P471S | K485R |
| KKKKREDLL | S464* | P471T | K485T |
| N457D | S464L | P471 > QTL | F486L |
| N457K | N465I | C472S | F486Y |
| N457S | N465K | C472W | P487L |
| N457_G460 > S | N465S | C472 > FF | P487Q |
| N457_G463 > R | N465T | L473I | P487R |
| N457_I459 > K | N465Y | L473V | P487S |
| N457_I459 > K | N465_P466ins27 | L473_E474insL | D488G |
| N457_P458 > TR | P466L | L473_E474insAC | D488H |
| N457_T462del | P466Q | L | D488N |
| N457_V461del | P466S | L473_L475 > FGV | D488_S490del |
| P458A | P466_N467insKL | WSLEL | M489I |
| P458L | LNPIGVTGSNP | E474A | M489V |
| P458R | N467H | E474K | S490P |
| P458S | N467K | E474Q | V491G |
| P458_G463 > R | N467T | E474V | V491L |
| P458_I459insM | K468* | L475* | V491M |
| NLWPVPHGLEDLLNP | K468R | L475F | I492F |
| P458_K468del | K468T | E476G | I492M |
| | K468_E469ins31 | E476K | I492T |
| | | E476Q | |
| E493K | Y508C | L523F | D538_S541 > A |
| E493Q | Y508H | L523V | D538del |
| E494* | Y508N | L523fs*1 | P539A |
| E494D | S509C | R524K | P539H |
| E494K | S509F | R524M | P539L |
| E494Q | H510Q | R524S | P539R |
| E494V | H510R | R524fs*36 | P539S |
| H495L | H510Y | E525* | P539T |
| H495N | A511P | E525A | P539del |
| H495Q | A511S | E525G | L540F |
| A496D | G512* | E525K | L540H |
| A496S | G512A | E525fs*35 | L540I |
| A496T | G512E | N526S | L540P |
| A496V | G512R | N526fs*34 | L540R |
| N497S | G512V | D527E | L540V |
| W498* | L513P | D527G | S541A |
| W498C | L513V | D527H | S541C |
| W498L | L513fs*5 | D527N | S541F |
| W498R | S514C | K528E | S541L |
| W498S | S514N | E529K | S541P |
| S499F | S514R | E529Q | S541T |
| S499Y | N515Y | Q530* | S541_E542insAISTRDRLS |
| V500L | R516I | Q530H | S541fs*1 |
| V500fs*9 | R516K | Q530K | E542A |
| S501F | R516T | Q530R | E542D |
| S501T | L517I | L531R | E542G |
| S501Y | L517P | A533T | E542I |
| R502* | L517R | I534N | E542K |
| R502G | L517V | I534V | E542L |
| R502Q | A518G | S535C | E542Q |
| E503G | A518P | S535F | E542R |
| E503K | A518S | S535Y | E542V |
| E503Q | A518T | T536A | I543V |
| E503del | R519K | T536K | I543_E545del |
| G505A | R519T | T536S | T544N |
| G505E | D520E | R537* | T544S |
| G505R | D520H | R537L | E545A |
| F506C | D520N | R537Q | E545D |
| F506L | N521D | D538A | E545G |
| F506V | N521K | D538E | E545K |
| S507G | N521S | D538G | E545L |
| S507I | E522* | D538H | E545N |
| S507R | E522K | D538N | E545P |
| S507T | E522Q | D538Y | E545Q |
| E545R | H554Q | R577T | E596Q |
| E545S | H554R | D578E | E596V |
| E545T | H554Y | D578H | E596fs*28 |
| E545V | R555G | D578N | Q597H |
| E545W | R555K | D578V | Q597K |
| E545_Q546 > DK | R555T | D578Y | Q597R |
| E545_Q546 > DL | H556D | E579* | Q597_A598 > HT |
| Q546E | Y557C | E579D | A598D |
| Q546H | Y557S | E579K | A598T |
| Q546K | C558F | E579Q | M599L |
| Q546L | C558S | V580A | M599_E600 > IK |
| Q546P | V559I | V580E | E600* |
| Q546R | V559L | V580L | E600A |
| E547* | T560I | A581T | E600K |
| E547D | T560P | A581V | L602M |
| E547G | T560S | Q582* | L602R |
| E547K | I561M | Q582L | L602_D603insT |
| E547Q | I561V | Q582R | D603N |
| K548N | P562L | M583I | D603Y |
| K548Q | P562S | Y584H | C604R |
| K548R | E563D | C585F | C604Y |
| D549G | E563K | C585G | N605H |
| D549H | I564S | L586F | N605K |
| D549N | P566L | L586M | N605S |
| D549Y | P566S | V587I | N605Y |
| D549fs*21 | K567Q | V587fs*10 | P607A |
| F550C | L569I | K588N | P607L |
| F550L | L570M | K588fs*8 | D608Y |
| F550V | L570P | D589E | P609G |
| L551I | S571C | D589H | P609H |
| L551P | V572A | D589Y | P609L |
| L551V | V572I | W590* | P609S |
| L551fs*8 | V572fs*9 | P591T | M610I |
| L551fs*9 | K573R | P592A | M610L |
| W552* | W574L | P592L | M610T |
| W552C | N575I | P592S | V611I |
| W552G | N575K | P592T | R612* |
| W552R | S576F | P592fs*32 | R612G |
| S553C | S576T | I593M | R612L |
| S553G | S576Y | I593N | R612P |
| S553N | R577G | I593V | R612Q |
| S553R | R577I | P595L | R612fs*1 |
| S553T | R577K | P595T | G613D |
| H554D | R577S | E596K | G613V |
| A615V | Y631C | A657P | E674Q |
| V616A | L632* | A657S | E674V |
| V616L | L632F | A657V | M675I |
| R617Q | I633L | L658F | M675T |
| R617W | Q634* | T659A | M675V |
| C618R | Q634E | T659N | H676R |
| C618W | Q634H | T659S | H676Y |
| L619S | V636A | N660D | H676fs*24 |
| L619V | V636L | N660S | N677H |
| K621* | V636R | Q661E | N677K |
| K621I | Q637* | Q661K | N677S |
| K621N | Q637K | Q661fs*11 | K678* |
| K621Q | Q637L | R662K | K678E |
| Y622* | V638A | R662M | K678T |
| L623F | V638I | R662S | T679R |
| L623I | K640Q | I663S | V680A |
| L623V | K640R | I663del | V680D |
| T624I | Y641* | G664E | V680I |
| T624P | E642K | G664W | V680fs*19 |
| T624S | E642Q | H665fs*7 | S681C |
| D625F | Q643H | F666C | S681I |
| D625H | Q643L | F666L | S681N |
| D625V | Q643R | F667L | S681R |
| D625Y | Y644* | W669* | S681T |
| D626E | Y644C | W669C | Q682* |
| D626G | L645F | W669G | Q682H |
| D626N | D646E | W669R | Q682R |
| D626Y | D646H | W669fs*6 | R683K |
| K627N | N647K | H670N | R683M |
| K627Q | N647S | H670Q | R683S |
| K627R | N647T | H670R | R683T |
| L628I | L648F | L671F | F684Y |
| L628P | L648V | L671I | G685S |
| L628R | L649F | L671V | G685V |
| L628V | V650A | L671fs*1 | L687F |
| S629C | R651K | K672I | L687I |
| S629F | R651S | K672N | L688M |
| S629P | L653* | S673C | E689K |
| S629Y | L653fs*2 | S673F | E689Q |
| Q630* | L653fs*8 | S673T | S690F |
| Q630E | L654V | E674A | S690Y |
| Q630H | K655N | E674D | Y691C |
| Q630P | K656N | E674G | C692* |
| Q630R | K656T | E674K | C692R |
| R693C | N714I | D725_Q728del | Q738fs*2 |
| R693G | N714K | D725_T727del | M739I |
| R693H | N714Y | D725del | R741* |
| R693L | L715V | E726* | R741Q |
| R693P | T716A | E726A | P742A |
| R693S | T716I | E726D | P742R |
| A694T | T716N | E726G | P742S |
| A694V | T716S | E726K | P742T |
| G696R | D717E | E726Q | D743E |
| G696W | D717H | E726V | D743G |
| M697fs*3 | D717N | T727A | D743H |
| Y698* | D717V | T727K | D743N |
| Y698C | D717_D725del | T727R | D743V |
| Y698H | I718L | T727S | F744I |
| L699F | I718V | T727del | F744L |
| L699V | I718_L719del | Q728K | F744V |
| H701Q | L719F | Q728R | M745I |
| L702V | L719I | K729N | M745L |
| R704S | L719R | V730A | M745T |
| R704T | L719V | V730I | D746A |
| R704W | K720E | V730L | D746G |
| Q705H | K720I | Q731E | D746H |
| Q705L | Q721E | Q731H | D746N |
| V706F | Q721H | Q731K | D746V |
| V706I | Q721K | Q731R | D746Y |
| E707* | Q721P | M732I | L748V |
| E707D | E722D | K733N | L748fs*5 |
| E707K | E722K | K733T | Q749* |
| E707Q | E722Q | F734I | Q749H |
| A708T | E722del | F734L | Q749K |
| M709I | K723N | F734V | G750A |
| M709V | K723R | L735I | G750C |
| E710K | K723T | L735S | G750D |
| K711E | K724T | L735V | G750E |
| K711N | K724del | V736A | G750S |
| K711Q | D725A | V736F | F751S |
| K711R | D725E | V736I | F751V |
| K711T | D725G | V736L | L752R |
| L712F | D725H | E737* | L752V |
| L712H | D725V | E737D | S753F |
| L712I | D725Y | E737K | S753T |
| L712V | D725_E726del | E737Q | S753Y |
| N714D | D725_K729del | Q738E | P754A |
| N714H | D725_Q728 > L | Q738R | P754S |
| P754T | R770L | S790A | T813A |
| P754del | R770Q | S790P | L814V |
| L755I | I771L | E791* | Q815* |
| L755Q | I771N | E791D | Q815R |
| L755V | I771S | E791Q | I816N |
| L755_N756 > P | I771T | L792* | I816S |
| N756K | M772I | L792F | I816T |
| N756S | M772V | L793V | I816V |
| N756Y | S773F | L793fs*5 | I817F |
| P757L | S773P | Q795* | I817V |
| P757S | S774C | Q795E | I817_I819del |
| P757fs*5 | S774F | Q795H | I817fs*14 |
| A758S | A775V | Q795R | R818C |
| A758T | K776R | N796H | R818G |
| A758V | R777K | N796I | R818H |
| H759D | R777S | E798D | R818L |
| H759N | R777fs*5 | E798K | I819N |
| H759Q | R777fs*22 | E798Q | I819V |
| H759Y | P778S | I799L | M820I |
| Q760* | L779V | I799M | M820L |
| Q760E | W780S | I799V | E821G |
| Q760L | L781F | I800F | E821K |
| Q760R | L781S | I800M | N822D |
| L761R | L781W | F801L | N822I |
| G762E | N782D | F801N | I823F |
| G762R | W783* | F801V | I823M |
| N763T | W783S | N803H | I823T |
| L764F | E784D | N803Y | I823V |
| L764I | N785I | G804R | W824* |
| L764P | N785S | D805G | W824F |
| L764V | P786A | D805N | W824G |
| R765G | P786Q | D805Y | Q825* |
| R765S | P786S | D806G | Q827E |
| L766P | P786T | D806Y | Q827K |
| L766fs*10 | D787G | L807S | G828D |
| E767G | D787V | L807V | L829F |
| E767K | I788F | R808L | L829I |
| E767Q | I788V | R808W | L829P |
| E768* | I788del | Q809* | D830G |
| E768D | M789I | Q809H | D830N |
| E768K | M789S | Q809K | D830V |
| E768Q | M789T | D810H | L831F |
| C769W | M789V | D810Y | L831I |
| R770* | S790* | M811I | L831V |
| R832* | I848T | G868C | E888D |
| R832L | E849* | G868S | E888G |
| R832Q | E849D | A869T | E888K |
| M833I | E849K | A869V | E888Q |
| M833V | E849Q | Q871L | I889L |
| M833fs*1 | V850A | F872L | I889M |
| L834S | V850G | F872V | I889T |
| L834V | V850M | N873D | Y890C |
| P835A | V851E | N873H | Y890N |
| P835L | R852* | N873K | A892V |
| P835T | R852G | N873S | A893S |
| Y836C | R852Q | S874N | A893T |
| G837C | N853K | H875N | A893V |
| G837D | N853S | H875R | A893fs*3 |
| G837N | S854A | H875Y | I894L |
| G837S | S854C | T876A | I894V |
| G837fs*30 | S854F | T876I | D895E |
| C838F | S854P | T876K | D895H |
| C838Y | H855L | L877I | D895N |
| L839V | H855R | L877V | D895Y |
| S840* | H855S | H878Y | L896M |
| S840L | H855T | Q879* | L896P |
| S840fs*27 | H855Y | Q879H | F897L |
| I841V | T856S | Q879K | F897fs*18 |
| I841fs*3 | I857L | Q879L | T898I |
| G842C | I857V | Q879R | T898P |
| G842S | I860M | L881I | T898fs*19 |
| D843G | I860V | L881V | R899C |
| D843N | Q861* | K882T | R899G |
| C844F | Q861E | D883E | R899H |
| C844R | Q861H | D883G | C901F |
| C844S | Q861K | D883H | C901S |
| C844Y | C862W | D883N | C901W |
| V845L | K863I | D883V | C901fs*19 |
| V845W | G864S | D883Y | A902P |
| V845_G846insC | G865D | K884* | A902T |
| V | G865S | K884N | A902V |
| V845fs*4 | L866F | N885S | G903* |
| G846* | L866V | K886E | G903A |
| L847F | L866W | K886N | G903E |
| L847H | K867I | K886R | Y904* |
| L847R | K867N | G887A | C905S |
| L847V | K867R | G887E | V906I |
| I848L | G868A | G887R | V906L |
| I848M | | | |
| A907V | D939N | F960L | E978Q |
| T908fs*15 | H940R | L961F | R979M |
| F909C | H940Y | I962M | R979S |
| F909L | K941N | I962T | F980V |
| I910L | K941R | V963M | Q981* |
| I910V | K942M | I964N | Q981E |
| L911F | K942N | I964fs*2 | Q981H |
| L911M | K943N | I964fs*22 | Q981K |
| L911fs*9 | K944I | S965N | Q981L |
| G912R | K944R | S965T | Q981R |
| G914E | K944del | K966I | E982D |
| G914R | F945C | K966R | E982K |
| D915N | F945I | G967A | E982Q |
| R916C | F945L | G967E | M983I |
| R916H | F945fs*4 | G967R | M983L |
| S919G | F945fs*12 | A968S | C984R |
| S919T | G946C | A968T | C984S |
| N920S | G946D | A968V | Y985C |
| I921N | G946V | Q969* | Y985D |
| I921V | Y947* | Q969H | Y985H |
| I921del | Y947F | Q969K | Y985N |
| M922I | R949* | Q969R | Y985S |
| V923L | R949Q | E970* | K986fs*5 |
| V923M | E950* | E970A | A987T |
| K924R | E950D | E970G | A987V |
| D925E | E950Q | E970K | Y988H |
| D926N | R951C | E970Q | Y988N |
| G927R | R951H | C971G | Y988S |
| G927V | R951L | T972R | L989Q |
| Q928R | V952A | K973N | L989R |
| L929F | V952L | T974K | L989V |
| L929M | F954Y | T974R | A990G |
| F930S | F954fs*7 | T974fs*3 | A990_I991 > V* |
| H931N | F954fs*11 | R975K | I991V |
| H931Y | V955F | R975T | I991fs*26 |
| I932L | L956F | E976* | R992* |
| D933N | T957I | E976D | R992Q |
| F934C | T957P | E976G | A995V |
| F934fs*23 | Q958* | E976K | N996I |
| H936R | Q958H | E976Q | N996K |
| F937fs*1 | Q958K | F977C | L997F |
| L938M | Q958L | F977L | L997H |
| L938fs*19 | Q958R | E978D | L997I |
| D939G | D959Y | E978K | L997V |
| F998L | E1012Q | L1028F | K1041* |
| F998fs*14 | Q1014K | L1028I | Q1042R |
| I999M | Q1014R | L1028S | Q1042fs*25 |
| I999R | S1015C | L1028V | 1043_1044MN > |
| I999V | S1015F | D1029E | IY |
| N1000H | S1015Y | D1029G | M1043I |
| N1000K | F1016I | D1029H | M1043L |
| N1000S | F1016L | D1029N | M1043T |
| L1001F | F1016S | D1029Y | M1043V |
| L1001I | F1016V | K1030* | M1043_N1044 > |
| L1001V | F1016Y | K1030E | IK |
| L1001fs*4 | D1017G | K1030R | M1043_N1044 > |
| L1001fs*17 | D1017H | T1031A | IR |
| F1002C | D1017V | T1031N | N1044D |
| F1002I | D1017Y | T1031P | N1044H |
| F1002L | D1018N | E1032* | N1044I |
| F1002V | I1019M | E1032A | N1044K |
| S1003K | I1019S | E1032D | N1044R |
| S1003L | I1019T | E1032K | N1044S |
| S1003fs*15 | A1020K | Q1033E | N1044T |
| M1004I | A1020S | Q1033K | N1044Y |
| M1004L | A1020T | Q1033P | D1045A |
| M1004R | A1020V | Q1033R | D1045G |
| M1004V | Y1021C | E1034G | D1045H |
| M1004del | Y1021F | E1034K | D1045N |
| M1005I | Y1021H | E1034Q | D1045V |
| M1005V | Y1021N | A1035T | D1045Y |
| L1006F | Y1021S | A1035V | A1046T |
| L1006H | I1022M | L1036F | H1047A |
| L1006R | I1022T | L1036K | H1047D |
| G1007C | I1022_R1023ins | L1036S | H1047I |
| G1007D | FLYVCTIAYI | E1037D | H1047L |
| G1007R | R1023* | E1037K | H1047N |
| S1008C | R1023L | E1037Q | H1047Q |
| G1009A | R1023P | Y1038C | H1047R |
| G1009E | R1023Q | Y1038F | H1047Y |
| G1009R | K1024N | Y1038H | H1047_H1048 > RR |
| M1010I | K1024T | Y1038N | H1048L |
| M1010I | T1025A | Y1038S | H1048N |
| M1010V | T1025I | F1039I | H1048R |
| P1011A | T1025N | F1039S | G1049A |
| P1011L | T1025S | M1040I | G1049C |
| P1011S | L1026P | M1040K | G1049D |
| E1012D | A1027T | M1040L | G1049R |
| | A1027V | | |
| G1049S | W1057C | H1065_*1069 > ? | L1067V |
| G1049W | I1058F | H1065_A1066 > L | L1067W |
| G1050A | I1058L | KLK | L1067_*1069 > F |
| G1050D | I1058M | H1065fs*? | KLKKN* |
| G1050S | H1060L | H1065fs*4 | L1067fs*4 |
| W1051C | T1061I | H1065fs*5 | L1067fs*5 |
| W1051L | T1061K | H1065fs*8 | L1067fs*6 |
| W1051fs*16 | I1062L | H1065fs*8 | L1067fs*7 |
| T1052A | I1062V | H1065fs*1+ | L1067fs*7 |
| T1052I | K1063* | H1065fs*15 | L1067fs*11+ |
| T1052K | Q1064H | H1065fs*6+ | L1067fs*20 |
| T1052R | Q1064_H1065in | A1066_*1069 > ? | L1067fs*5+ |
| T1053fs*15 | sQWTTKMDWIF | A1066_L1067ins | N1068K |
| K1054T | HTIKQ | CV | N1068fs*1 |
| K1054_M1055 > | Q1064fs*6 | A1066fs*2+ | N1068fs*3 |
| NGLDLPHN*TAC | Q1064fs*7 | A1066fs*4 | N1068fs*4 |
| IEM | Q1064fs*9 | A1066fs*5 | N1068fs*5 |
| M1055I | Q1064fs*24 | A1066fs*5+ | N1068fs*5 |
| M1055L | Q1064fs*5+ | A1066fs*8 | N1068fs*7 |
| M1055V | H1065L | A1066I | N1068fs*1+ |
| D1056H | H1065Q | A1066L | N1068fs*10 |
| D1056N | H1065R | A1066L | N1068fs*11 |
| W1057* | H1065Y | L1067F | N1068fs*21 |

As used herein, the term “PI3Kα-mediated” disorders, diseases, and/or conditions means any disease or other deleterious condition in which PI3Kα or a mutant thereof is known to play a role. Accordingly, another embodiment of the present disclosure relates to treating or lessening the severity of one or more diseases in which PI3Kα, or a mutant thereof, is known to play a role. Such PI3Kα-mediated disorders include, but are not limited to, cellular proliferative disorders (e.g., cancer). In some embodiments, the PI3Kα-mediated disorder is a disorder mediated by a mutant PI3Kα. In some embodiments, the PI3Kα-mediated disorder is a disorder mediated by a PI3Kα containing at least one of the following mutations: H1047R, E542K, and E545K.

In some embodiments, the present disclosure provides a method for treating a cellular proliferative disease, said method comprising administering to a patient in need thereof a therapeutically effective amount of a compound of the present disclosure, or a pharmaceutically acceptable salt thereof, or a pharmaceutically acceptable composition of either of the foregoing. In some embodiments, the present disclosure provides a method for treating a cellular proliferative disease, said method comprising administering to a patient in need thereof, a therapeutically effective amount of a compound of the present disclosure, or a pharmaceutically acceptable composition thereof.

In some embodiments, the method of treatment comprises the steps of: (i) identifying a subject in need of such treatment; (ii) providing a disclosed compound, or a pharmaceutically acceptable salt thereof; and (iii) administering said provided compound in a therapeutically effective amount to treat, suppress and/or prevent the disease state or condition in a subject in need of such treatment. In some embodiments, the subject has a mutant PI3Kα. In some embodiments, the subject has PI3Kα containing at least one of the following mutations: H1047R, E542K, and E545K.

In some embodiments, the method of treatment comprises the steps of: (i) identifying a subject in need of such treatment; (ii) providing a composition comprising a disclosed compound, or a pharmaceutically acceptable salt thereof; and (iii) administering said composition in a therapeutically effective amount to treat, suppress and/or prevent the disease state or condition in a subject in need of such treatment. In some embodiments, the subject has a mutant PI3Kα. In some embodiments, the subject has PI3Kα containing at least one of the following mutations: H1047R, E542K, and E545K.

Another aspect of the disclosure provides a compound according to the definitions herein, or a pharmaceutically acceptable salt thereof, or a pharmaceutical composition of either of the foregoing, for use in the treatment of a disorder described herein. Another aspect of the disclosure provides the use of a compound according to the definitions herein, or a pharmaceutically acceptable salt thereof, or a pharmaceutical composition of either of the foregoing, for the treatment of a disorder described herein. Similarly, the disclosure provides the use of a compound according to the definitions herein, or a pharmaceutically acceptable salt thereof, for the preparation of a medicament for the treatment of a disorder described herein.

### Cellular Proliferative Diseases

In some embodiments, the disorder is a cellular proliferative disease. In some embodiments, the cellular proliferative disease is cancer. In some embodiments, the cancer is a tumor. In some embodiments, the cancer is a solid tumor. In some embodiments, the cellular proliferative disease is a tumor and/or cancerous cell growth. In some embodiments, the cellular proliferative disease is a tumor. In some embodiments, the cellular proliferative disease is a solid tumor. In some embodiments, the cellular proliferative disease is a cancerous cell growth.

In some embodiments, the cancer is selected from sarcoma; lung; bronchus; prostate; breast (including sporadic breast cancers and sufferers of Cowden disease); pancreas; gastrointestinal; colon; rectum; carcinoma; colon carcinoma; adenoma; colorectal adenoma; thyroid; liver; intrahepatic bile duct; hepatocellular; adrenal gland; stomach; gastric; glioma; glioblastoma; endometrial; melanoma; kidney; renal pelvis; urinary bladder; uterine corpus; uterine cervix; vagina; ovary (including clear cell ovarian cancer); multiple myeloma; esophagus; a leukemia; acute myelogenous leukemia; chronic myelogenous leukemia; lymphocytic leukemia; myeloid leukemia; brain; a carcinoma of the brain; oral cavity and pharynx; larynx; small intestine; non-Hodgkin lymphoma; villous colon adenoma; a neoplasia; a neoplasia of epithelial character; lymphoma; a mammary carcinoma; basal cell carcinoma; squamous cell carcinoma; actinic keratosis; neck; head; polycythemia vera; essential thrombocythemia; myelofibrosis with myeloid metaplasia; and Waldenstrom macroglobulinemia.

In some embodiments, the cancer is selected from lung; bronchus; prostate; breast (including sporadic breast cancers and Cowden disease); pancreas; gastrointestinal; colon; rectum; thyroid; liver; intrahepatic bile duct; hepatocellular; adrenal gland; stomach; gastric; endometrial; kidney; renal pelvis; urinary bladder; uterine corpus; uterine cervix; vagina; ovary (including clear cell ovarian cancer); esophagus; a leukemia; acute myelogenous leukemia; chronic myelogenous leukemia; lymphocytic leukemia; myeloid leukemia; brain; oral cavity and pharynx; larynx; small intestine; neck; and head. In some embodiments, the cancer is selected from sarcoma; carcinoma; colon carcinoma; adenoma; colorectal adenoma; glioma; glioblastoma; melanoma; multiple myeloma; a carcinoma of the brain; non-Hodgkin lymphoma; villous colon adenoma; a neoplasia; a neoplasia of epithelial character; lymphoma; a mammary carcinoma; basal cell carcinoma; squamous cell carcinoma; actinic keratosis; polycythemia vera; essential thrombocythemia; myelofibrosis with myeloid metaplasia; and Waldenstrom macroglobulinemia.

In some embodiments, the cancer is selected from lung; bronchus; prostate; breast (including sporadic breast cancers and Cowden disease); pancreas; gastrointestinal; colon; rectum; thyroid; liver; intrahepatic bile duct; hepatocellular; adrenal gland; stomach; gastric; endometrial; kidney; renal pelvis; urinary bladder; uterine corpus; uterine cervix; vagina; ovary (including clear cell ovarian cancer); esophagus; brain; oral cavity and pharynx; larynx; small intestine; neck; and head. In some embodiments, the cancer is a leukemia. In some embodiments, the cancer is acute myelogenous leukemia; chronic myelogenous leukemia; lymphocytic leukemia; or myeloid leukemia.

In some embodiments, the cancer is breast cancer (including sporadic breast cancers and Cowden disease). In some embodiments, the cancer is breast cancer. In some embodiments, the cancer is ER+/HER2− breast cancer. In some embodiments, the cancer is ER+/HER2− breast cancer, and the subject is intolerant to, or ineligible for, treatment with alpelisib. In some embodiments, the cancer is sporadic breast cancer. In some embodiments, the cancer is Cowden disease.

In some embodiments, the cancer is ovarian cancer. In some embodiments, the ovarian cancer is clear cell ovarian cancer.

In some embodiments, the cellular proliferative disease has mutant PI3Kα. In some embodiments, the cancer has mutant PI3Kα. In some embodiments, the breast cancer has mutant PI3Kα. In some embodiments, the ovarian cancer has mutant PI3Kα.

In some embodiments, the cellular proliferative disease has PI3Kα containing at least one of the following mutations: H1047R, E542K, and E545K. In some embodiments, the cancer has PI3Kα containing at least one of the following mutations: H1047R, E542K, and E545K. In some embodiments, the breast cancer has PI3Kα containing at least one of the following mutations: H1047R, E542K, and E545K. In some embodiments, the ovarian cancer has PI3Kα containing at least one of the following mutations: H1047R, E542K, and E545K.

In some embodiments, the cancer is adenoma; carcinoma; sarcoma; glioma; glioblastoma; melanoma; multiple myeloma; or lymphoma. In some embodiments, the cancer is a colorectal adenoma or avillous colon adenoma. In some embodiments, the cancer is colon carcinoma; a carcinoma of the brain; a mammary carcinoma; basal cell carcinoma; or a squamous cell carcinoma. In some embodiments, the cancer is a neoplasia or a neoplasia of epithelial character. In some embodiments, the cancer is non-Hodgkin lymphoma. In some embodiments, the cancer is actinic keratosis; polycythemia vera; essential thrombocythemia; myelofibrosis with myeloid metaplasia; or Waldenstrom macroglobulinemia.

In some embodiments, the cellular proliferative disease displays overexpression or amplification of PI3Kα, somatic mutation of PIK3CA, germline mutations or somatic mutation of PTEN, or mutations and translocation of p85a that serve to up-regulate the p85-p110 complex. In some embodiments, the cellular proliferative disease displays overexpression or amplification of PI3Kα. In some embodiments, the cellular proliferative disease displays somatic mutation of PIK3CA. In some embodiments, the cellular proliferative disease displays germline mutations or somatic mutation of PTEN. In some embodiments, the cellular proliferative disease displays mutations and translocation of p85a that serve to up-regulate the p85-p110 complex.

### Additional Disorders

In some embodiments, the PI3Kα-mediated disorder is selected from the group consisting of: polycythemia vera, essential thrombocythemia, myelofibrosis with myeloid metaplasia, asthma, COPD, ARDS, PROS (PI3K-related overgrowth syndrome), venous malformation, Loffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma, eosinophil-related disorders affecting the airways occasioned by drug-reaction, psoriasis, contact dermatitis, atopic dermatitis, alopecia greata, erythema multiforme, dermatitis herpetiformis, scleroderma, vitiligo, hypersensitivity angiitis, urticaria, bullous pemphigoid, lupus erythematosus, pemphisus, epidermolysis bullosa acquisita, autoimmune haematogical disorders (e.g., haemolytic anaemia, aplastic anaemia, pure red cell anaemia and idiopathic thrombocytopenia), systemic lupus erythematosus, polychondritis, Wegener granulomatosis, dermatomyositis, chronic active hepatitis, myasthenia gravis, Steven-Johnson syndrome, idiopathic sprue, autoimmune inflammatory bowel disease (e.g., ulcerative colitis and Crohn's disease), endocrine opthalmopathy, Graves' disease, sarcoidosis, alveolitis, chronic hypersensitivity pneumonitis, multiple sclerosis, primary biliary cirrhosis, uveitis (anterior and posterior), interstitial lung fibrosis, psoriatic arthritis, glomerulonephritis, cardiovascular diseases, atherosclerosis, hypertension, deep venous thrombosis, stroke, myocardial infarction, unstable angina, thromboembolism, pulmonary embolism, thrombolytic diseases, acute arterial ischemia, peripheral thrombotic occlusions, and coronary artery disease, reperfusion injuries, retinopathy, such as diabetic retinopathy or hyperbaric oxygen-induced retinopathy, and conditions characterized by elevated intraocular pressure or secretion of ocular aqueous humor, such as glaucoma.

In some embodiments, the PI3Kα-mediated disorder is polycythemia vera, essential thrombocythemia, or myelofibrosis with myeloid metaplasia. In some embodiments, the PI3Kα-mediated disorder is asthma, COPD, ARDS, PROS (PI3K-related overgrowth syndrome), venous malformation, Loffler's syndrome, eosinophilic pneumonia, parasitic (in particular metazoan) infestation (including tropical eosinophilia), or bronchopulmonary aspergillosis. In some embodiments, the PI3Kα-mediated disorder is polyarteritis nodosa (including Churg-Strauss syndrome), eosinophilic granuloma, eosinophil-related disorders affecting the airways occasioned by drug-reaction, psoriasis, contact dermatitis, atopic dermatitis, alopecia greata, erythema multiforme, dermatitis herpetiformis, or scleroderma. In some embodiments, the PI3Kα-mediated disorder is vitiligo, hypersensitivity angiitis, urticaria, bullous pemphigoid, lupus erythematosus, pemphisus, epidermolysis bullosa acquisita, or autoimmune haematogical disorders (e.g., haemolytic anaemia, aplastic anaemia, pure red cell anaemia and idiopathic thrombocytopenia). In some embodiments, the PI3Kα-mediated disorder is systemic lupus erythematosus, polychondritis, scleroderma, Wegener granulomatosis, dermatomyositis, chronic active hepatitis, myasthenia gravis, Steven-Johnson syndrome, idiopathic sprue, or autoimmune inflammatory bowel disease (e.g., ulcerative colitis and Crohn's disease).

In some embodiments, the PI3Kα-mediated disorder is endocrine opthalmopathy, Graves' disease, sarcoidosis, alveolitis, chronic hypersensitivity pneumonitis, multiple sclerosis, primary biliary cirrhosis, uveitis (anterior and posterior), interstitial lung fibrosis, or psoriatic arthritis. In some embodiments, the PI3Kα-mediated disorder is glomerulonephritis, cardiovascular diseases, atherosclerosis, hypertension, deep venous thrombosis, stroke, myocardial infarction, unstable angina, thromboembolism, pulmonary embolism, thrombolytic diseases, acute arterial ischemia, peripheral thrombotic occlusions, and coronary artery disease, or reperfusion injuries. In some embodiments, the PI3Kα-mediated disorder is retinopathy, such as diabetic retinopathy or hyperbaric oxygen-induced retinopathy, and conditions characterized by elevated intraocular pressure or secretion of ocular aqueous humor, such as glaucoma.

### Routes of Administration and Dosage Forms

The compounds and compositions, according to the methods of the present disclosure, may be administered using any amount and any route of administration effective for treating or lessening the severity of the disorder (e.g., a proliferative disorder). The e^{YA}ct amount required will vary from subject to subject, depending on the species, age, and general condition of the subject, the severity of the infection, the particular agent, its mode of administration, and the like. Compounds of the disclosure are preferably formulated in unit dosage form for ease of administration and uniformity of dosage. The expression “unit dosage form” as used herein refers to a physically discrete unit of agent appropriate for the patient to be treated. It will be understood, however, that the total daily usage of the compounds and compositions of the present disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this disclosure can be administered to humans and other animals orally, rectally, parenterally, intracisternally, intravaginally, intraperitoneally, topically (as by powders, ointments, or drops), bucally, as an oral or nasal spray, or the like. In certain embodiments, the compounds of the disclosure may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this disclosure with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose, or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Dosage Amounts and Regimens

In accordance with the methods of the present disclosure, the compounds of the disclosure are administered to the subject in a therapeutically effective amount, e.g., to reduce or ameliorate symptoms of the disorder in the subject. This amount is readily determined by the skilled artisan, based upon known procedures, including analysis of titration curves established in vivo and methods and assays disclosed herein.

In some embodiments, the methods comprise administration of a therapeutically effective dosage of the compounds of the disclosure. In some embodiments, the therapeutically effective dosage is at least about 0.0001 mg/kg body weight, at least about 0.001 mg/kg body weight, at least about 0.01 mg/kg body weight, at least about 0.05 mg/kg body weight, at least about 0.1 mg/kg body weight, at least about 0.25 mg/kg body weight, at least about 0.3 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 350 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight. It will be recognized that any of the dosages listed herein may constitute an upper or lower dosage range and may be combined with any other dosage to constitute a dosage range comprising an upper and lower limit.

In some embodiments, the therapeutically effective dosage is in the range of about 0.1 mg to about 10 mg/kg body weight, about 0.1 mg to about 6 mg/kg body weight, about 0.1 mg to about 4 mg/kg body weight, or about 0.1 mg to about 2 mg/kg body weight.

In some embodiments the therapeutically effective dosage is in the range of about 1 to 500 mg, about 2 to 150 mg, about 2 to 120 mg, about 2 to 80 mg, about 2 to 40 mg, about 5 to 150 mg, about 5 to 120 mg, about 5 to 80 mg, about 10 to 150 mg, about 10 to 120 mg, about 10 to 80 mg, about 10 to 40 mg, about 20 to 150 mg, about 20 to 120 mg, about 20 to 80 mg, about 20 to 40 mg, about 40 to 150 mg, about 40 to 120 mg or about 40 to 80 mg.

In some embodiments, the methods comprise a single dosage or administration (e.g., as a single injection or deposition). Alternatively, in some embodiments, the methods comprise administration once daily, twice daily, three times daily or four times daily to a subject in need thereof for a period of from about 2 to about 28 days, or from about 7 to about 10 days, or from about 7 to about 15 days, or longer. In some embodiments, the methods comprise chronic administration. In yet other embodiments, the methods comprise administration over the course of several weeks, months, years, or decades. In still other embodiments, the methods comprise administration over the course of several weeks. In still other embodiments, the methods comprise administration over the course of several months. In still other embodiments, the methods comprise administration over the course of several years. In still other embodiments, the methods comprise administration over the course of several decades.

The dosage administered can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion. These are all readily determined and may be used by the skilled artisan to adjust or titrate dosages and/or dosing regimens.

### Inhibition of Protein Kinases

According to one embodiment, the disclosure relates to a method of inhibiting protein kinase activity in a biological sample comprising the step of contacting said biological sample with a compound of this disclosure, or a composition comprising said compound. According to another embodiment, the disclosure relates to a method of inhibiting activity of a PI3K, or a mutant thereof, in a biological sample comprising the step of contacting said biological sample with a compound of this disclosure, or a composition comprising said compound. According to another embodiment, the disclosure relates to a method of inhibiting activity of PI3Kα, or a mutant thereof, in a biological sample comprising the step of contacting said biological sample with a compound of this disclosure, or a composition comprising said compound. In some embodiments, the PI3Kα is a mutant PI3Kα. In some embodiments, the PI3Kα contains at least one of the following mutations: H1047R, E542K, and E545K.

In another embodiment, the disclosure provides a method of selectively inhibiting PI3Kα over one or both of PI3Kδ and PI3Kγ. In some embodiments, a compound of the present disclosure is more than 5-fold selective over PI3Kδ and PI3Kγ. In some embodiments, a compound of the present disclosure is more than 10-fold selective over PI3Kδ and PI3Kγ. In some embodiments, a compound of the present disclosure is more than 50-fold selective over PI3Kδ and PI3Kγ. In some embodiments, a compound of the present disclosure is more than 100-fold selective over PI3Kδ and PI3Kγ. In some embodiments, a compound of the present disclosure is more than 200-fold selective over PI3Kδ and PI3Kγ. In some embodiments, the PI3Kα is a mutant PI3Kα. In some embodiments, the PI3Kα contains at least one of the following mutations: H1047R, E542K, and E545K.

In another embodiment, the disclosure provides a method of selectively inhibiting a mutant PI3Kα over a wild-type PI3Kα. In some embodiments, a compound of the present disclosure is more than 5-fold selective for mutant PI3Kα over wild-type PI3Kα. In some embodiments, a compound of the present disclosure is more than 10-fold selective for mutant PI3Kα over wild-type PI3Kα. In some embodiments, a compound of the present disclosure is more than 50-fold selective for mutant PI3Kα over wild-type PI3Kα. In some embodiments, a compound of the present disclosure is more than 100-fold selective for mutant PI3Kα over wild-type PI3Kα. In some embodiments, a compound of the present disclosure is more than 200-fold selective for mutant PI3Kα over wild-type PI3Kα. In some embodiments, the mutant PI3Kα contains at least one of the following mutations: H1047R, E542K, and E545K.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of activity of a PI3K (for example, PI3Kα, or a mutant thereof) in a biological sample is useful for a variety of purposes that are known to one of skill in the art. Examples of such purposes include, but are not limited to, blood transfusion, organ-transplantation, biological specimen storage, and biological assays.

Another embodiment of the present disclosure relates to a method of inhibiting protein kinase activity in a patient comprising the step of administering to said patient a compound of the present disclosure, or a composition comprising said compound.

According to another embodiment, the disclosure relates to a method of inhibiting activity of a PI3K, or a mutant thereof, in a patient comprising the step of administering to said patient a compound of the present disclosure, or a composition comprising said compound. In some embodiments, the disclosure relates to a method of inhibiting activity of PI3Kα, or a mutant thereof, in a patient comprising the step of administering to said patient a compound of the present disclosure, or a composition comprising said compound. In some embodiments, the PI3Kα is a mutant PI3Kα. In some embodiments, the PI3Kα contains at least one of the following mutations: H1047R, E542K, and E545K.

According to another embodiment, the present disclosure provides a method for treating a disorder mediated by a PI3K, or a mutant thereof, in a patient in need thereof, comprising the step of administering to said patient a compound according to the present disclosure or pharmaceutically acceptable composition thereof. In some embodiments, the present disclosure provides a method for treating a disorder mediated by PI3Kα, or a mutant thereof, in a patient in need thereof, comprising the step of administering to said patient a compound according to the present disclosure or pharmaceutically acceptable composition thereof. In some embodiments, the PI3Kα is a mutant PI3Kα. In some embodiments, the PI3Kα contains at least one of the following mutations: H1047R, E542K, and E545K.

According to another embodiment, the present disclosure provides a method of inhibiting signaling activity of PI3Kα, or a mutant thereof, in a subject, comprising administering a therapeutically effective amount of a compound according to the present disclosure, or a pharmaceutically acceptable composition thereof, to a subject in need thereof. In some embodiments, the present disclosure provides a method of inhibiting PI3Kα signaling activity in a subject, comprising administering a therapeutically effective amount of a compound according to the present disclosure, or a pharmaceutically acceptable composition thereof, to a subject in need thereof. In some embodiments, the PI3Kα is a mutant PI3Kα. In some embodiments, the PI3Kα contains at least one of the following mutations: H1047R, E542K, and E545K. In some embodiments, the subject has a mutant PI3Kα. In some embodiments, the subject has PI3Kα containing at least one of the following mutations: H1047R, E542K, and E545K.

The compounds described herein can also inhibit PI3Kα function through incorporation into agents that catalyze the destruction of PI3Kα. For example, the compounds can be incorporated into proteolysis targeting chimeras (PROTACs). A PROTAC is a bifunctional molecule, with one portion capable of engaging an E3 ubiquitin ligase, and the other portion having the ability to bind to a target protein meant for degradation by the cellular protein quality control machinery. Recruitment of the target protein to the specific E3 ligase results in its tagging for destruction (i.e., ubiquitination) and subsequent degradation by the proteasome. Any E3 ligase can be used. The portion of the PROTAC that engages the E3 ligase is connected to the portion of the PROTAC that engages the target protein via a linker which consists of a variable chain of atoms. Recruitment of PI3Kα to the E3 ligase will thus result in the destruction of the PI3Kα protein. The variable chain of atoms can include, for example, rings, heteroatoms, and/or repeating polymeric units. It can be rigid or flexible. It can be attached to the two portions described above using standard techniques in the art of organic synthesis.

### Combination Therapies

Depending upon the particular disorder, condition, or disease, to be treated, additional therapeutic agents, that are normally administered to treat that condition, may be administered in combination with compounds and compositions of this disclosure. As used herein, additional therapeutic agents that are normally administered to treat a particular disease, or condition, are known as “appropriate for the disease, or condition, being treated.”

Additionally, PI3K serves as a second messenger node that integrates parallel signaling pathways, and evidence is emerging that the combination of a PI3K inhibitor with inhibitors of other pathways will be useful in treating cancer and cellular proliferative diseases.

Accordingly, in certain embodiments, the method of treatment comprises administering the compound or composition of the disclosure in combination with one or more additional therapeutic agents. In certain other embodiments, the methods of treatment comprise administering the compound or composition of the disclosure as the only therapeutic agent.

Approximately 20-30% of human breast cancers overexpress Her-2/neu-ErbB2, the target for the drug trastuzumab. Although trastuzumab has demonstrated durable responses in some patients expressing Her2/neu-ErbB2, only a subset of these patients respond. Recent work has indicated that this limited response rate can be substantially improved by the combination of trastuzumab with inhibitors of PI3K or the PI13K/AKT pathway (Chan et al., Breast Can. Res. Treat. 91:187 (2005), Woods Ignatoski et al., Brit. J. Cancer 82:666 (2000), Nagata et al., Cancer Cell 6:117 (2004)). Accordingly, in certain embodiments, the method of treatment comprises administering the compound or composition of the disclosure in combination with trastuzumab. In certain embodiments, the cancer is a human breast cancer that overexpresses Her-2/neu-ErbB2.

A variety of human malignancies express activating mutations or increased levels of Her1/EGFR and a number of antibody and small molecule inhibitors have been developed against this receptor tyrosine kinase including tarceva, gefitinib and erbitux. However, while EGFR inhibitors demonstrate anti-tumor activity in certain human tumors (e.g., NSCLC), they fail to increase overall patient survival in all patients with EGFR-expressing tumors. This may be rationalized by the fact that many downstream targets of Her1/EGFR are mutated or deregulated at high frequencies in a variety of malignancies, including the PI3K/Akt pathway.

For example, gefitinib inhibits the growth of an adenocarcinoma cell line in in vitro assays. Nonetheless, sub-clones of these cell lines can be selected that are resistant to gefitinib that demonstrate increased activation of the PI3/Akt pathway. Down-regulation or inhibition of this pathway renders the resistant sub-clones sensitive to gefitinib (Kokubo et al., Brit. J. Cancer 92:1711 (2005)). Furthermore, in an in vitro model of breast cancer with a cell line that harbors a PTEN mutation and over-expresses EGFR inhibition of both the PI3K/Akt pathway and EGFR produced a synergistic effect (She et al., Cancer Cell 8:287-297 (2005)). These results indicate that the combination of gefitinib and PI3K/Akt pathway inhibitors would be an attractive therapeutic strategy in cancer.

Accordingly, in certain embodiments, the method of treatment comprises administering the compound or composition of the disclosure in combination with an inhibitor of Her1/EGFR. In certain embodiments, the method of treatment comprises administering the compound or composition of the disclosure in combination with one or more of tarceva, gefitinib, and erbitux. In certain embodiments, the method of treatment comprises administering the compound or composition of the disclosure in combination with gefitinib. In certain embodiments, the cancer expresses activating mutations or increased levels of Her1/EGFR.

The combination of AEE778 (an inhibitor of Her-2/neu/ErbB2, VEGFR and EGFR) and RAD001 (an inhibitor of mTOR, a downstream target of Akt) produced greater combined efficacy that either agent alone in a glioblastoma xenograft model (Goudar et al., Mol. Cancer. Ther. 4:101-112 (2005)).

Anti-estrogens, such as tamoxifen, inhibit breast cancer growth through induction of cell cycle arrest that requires the action of the cell cycle inhibitor p27Kip. Recently, it has been shown that activation of the Ras-Raf-MAP Kinase pathway alters the phosphorylation status of p27Kip such that its inhibitory activity in arresting the cell cycle is attenuated, thereby contributing to anti-estrogen resistance (Donovan, et al, J. Biol. Chem. 276:40888, (2001)). As reported by Donovan et al., inhibition of MAPK signaling through treatment with MEK inhibitor reversed the aberrant phosphorylation status of p27 in hormone refractory breast cancer cell lines and in so doing restored hormone sensitivity. Similarly, phosphorylation of p27Kip by Aid also abrogates its role to arrest the cell cycle (Viglietto et al., Nat. Med. 8:1145 (2002)).

Accordingly, in certain embodiments, the method of treatment comprises administering the compound or composition of the disclosure in combination with a treatment for a hormone-dependent cancer. In certain embodiments, the method of treatment comprises administering the compound or composition of the disclosure in combination with tamoxifen. In certain embodiments, the cancer is a hormone dependent cancer, such as breast and prostate cancers. By this use, it is aimed to reverse hormone resistance commonly seen in these cancers with conventional anticancer agents.

In hematological cancers, such as chronic myelogenous leukemia (CML), chromosomal translocation is responsible for the constitutively activated BCR-Abl tyrosine kinase. The afflicted patients are responsive to imatinib, a small molecule tyrosine kinase inhibitor, as a result of inhibition of Abl kinase activity. However, many patients with advanced stage disease respond to imatinib initially, but then relapse later due to resistance-conferring mutations in the Abl kinase domain. In vitro studies have demonstrated that BCR-Abl employs the Ras-Raf kinase pathway to elicit its effects. In addition, inhibiting more than one kinase in the same pathway provides additional protection against resistance-conferring mutations.

Accordingly, in another aspect, the compounds and compositions of the disclosure are used in combination with at least one additional agent selected from the group of kinase inhibitors, such as imatinib, in the treatment of hematological cancers, such as chronic myelogenous leukemia (CML). By this use, it is aimed to reverse or prevent resistance to said at least one additional agent.

Because activation of the PI3K/Akt pathway drives cell survival, inhibition of the pathway in combination with therapies that drive apoptosis in cancer cells, including radiotherapy and chemotherapy, will result in improved responses (Ghobrial et al., CA Cancer J. Clin 55:178-194 (2005)). As an example, combination of PI3 kinase inhibitor with carboplatin demonstrated synergistic effects in both in vitro proliferation and apoptosis assays as well as in in vivo tumor efficacy in a xenograft model of ovarian cancer (Westfall and Skinner, Mol. Cancer Ther. 4:1764-1771 (2005)).

In some embodiments, the one or more additional therapeutic agents is selected from antibodies, antibody-drug conjugates, kinase inhibitors, immunomodulators, and histone deacetylase inhibitors. Synergistic combinations with PIK3CA inhibitors and other therapeutic agents are described in, for example, Castel et al., Mol. Cell Oncol. (2014)1(3) e963447.

In some embodiments, the one or more additional therapeutic agent is selected from the following agents, or a pharmaceutically acceptable salt thereof: BCR-ABL inhibitors (see, e.g., Ultimo et al. Oncotarget (2017) 8 (14) 23213-23227.): e.g., imatinib, inilotinib, nilotinib, dasatinib, bosutinib, ponatinib, bafetinib, danusertib, saracatinib, PF03814735; ALK inhibitors (see, e.g., Yang et al. Tumour Biol. (2014) 35 (10) 9759-67): e.g., crizotinib, NVP-TAE684, ceritinib, alectinib, brigatinib, entrecinib, lorlatinib; BRAF inhibitors (see, e.g., Silva et al. Mol. Cancer Res. (2014) 12, 447-463): e.g., vemurafenib, dabrafenib; FGFR inhibitors (see, e.g., Packer et al. Mol. Cancer Ther. (2017) 16(4) 637-648): e.g., infigratinib, dovitinib, erdafitinib, TAS-120, pemigatinib, BLU-554, AZD4547; FLT3 inhibitors: e.g., sunitinib, midostaurin, tanutinib, sorafenib, lestaurtinib, quizartinib, and crenolanib; MEK Inhibitors (see, e.g., Jokinen et al. Ther. Adv. Med. Oncol. (2015) 7(3) 170-180): e.g., trametinib, cobimetinib, binimetinib, selumetinib; ERK inhibitors: e.g., ulixertinib, MK 8353, LY 3214996; KRAS inhibitors: e.g., AMG-510, MRTX849, ARS-3248; Tyrosine kinase inhibitors (see, e.g., Makhov et al. Mol. Cancer. Ther. (2012) 11(7) 1510-1517): e.g., erlotinib, linifanib, sunitinib, pazopanib; Epidermal growth factor receptor (EGFR) inhibitors (see, e.g., She et al. BMC Cancer (2016) 16, 587): gefitnib, osimertinib, cetuximab, panitumumab; HER2 receptor inhibitors (see, e.g., Lopez et al. Mol. Cancer Ther. (2015) 14(11) 2519-2526): e.g., trastuzumab, pertuzumab, neratinib, lapatinib, lapatinib; MET inhibitors (see, e.g., Hervieu et al. Front. Mol. Biosci. (2018) 5, 86): e.g., crizotinib, cabozantinib; CD20 antibodies: e.g., rituximab, tositumomab, ofatumumab; DNA Synthesis inhibitors: e.g., capecitabine, gemcitabine, nelarabine, hydroxycarbamide; Antineoplastic agents (see, e.g., Wang et al. Cell Death & Disease (2018) 9, 739): e.g., oxaliplatin, carboplatin, cisplatin; Immunomodulators: e.g., afutuzumab, lenalidomide, thalidomide, pomalidomide; CD40 inhibitors: e.g., dacetuzumab; Pro-apoptotic receptor agonists (PARAs): e.g., dulanermin; Heat Shock Protein (HSP) inhibitors (see, e.g., Chen et al. Oncotarget (2014) 5 (9). 2372-2389): e.g., tanespimycin; Hedgehog antagonists (see, e.g., Chaturvedi et al. Oncotarget (2018) 9 (24), 16619-16633): e.g., vismodegib; Proteasome inhibitors (see, e.g., Lin et al. Int. J. Oncol. (2014) 44 (2), 557-562): e.g., bortezomib; PI3K inhibitors: e.g., pictilisib, dactolisib, alpelisib, buparlisib, taselisib, idelalisib, duvelisib, umbralisib; SHP2 inhibitors (see, e.g., Sun et al. Am. J. Cancer Res. (2019) 9 (1), 149-159: e.g., SHP099, RMC-4550, RMC-4630); BCL-2 inhibitors (see, e.g., Bojarczuk et al. Blood (2018) 133 (1), 70-80): e.g., venetoclax; Aromatase inhibitors (see, e.g., Mayer et al. Clin. Cancer Res. (2019) 25 (10), 2975-2987): exemestane, letrozole, anastrozole, fulvestrant, tamoxifen; mTOR inhibitors (see, e.g., Woo et al. Oncogenesis (2017) 6, e385): e.g., temsirolimus, ridaforolimus, everolimus, sirolimus; CTLA-4 inhibitors (see, e.g., O'Donnell et al. (2018) 48, 91-103): e.g., tremelimumab, ipilimumab; PD1 inhibitors (see O'Donnell, supra): e.g., nivolumab, pembrolizumab; an immunoadhesin; Other immune checkpoint inhibitors (see, e.g., Zappasodi et al. Cancer Cell (2018) 33, 581-598, where the term “immune checkpoint” refers to a group of molecules on the cell surface of CD4 and CD8 T cells. Immune checkpoint molecules include, but are not limited to, Programmed Death 1 (PD-1), Cytotoxic T-Lymphocyte Antigen 4 (CTLA-4), B7H1, B7H4, OX-40, CD 137, CD40, and LAG3. Immunotherapeutic agents which can act as immune checkpoint inhibitors useful in the methods of the present disclosure, include, but are not limited to, inhibitors of PD-L1, PD-L2, CTLA4, TIM3, LAG3, VISTA, BTLA, TIGIT, LAIR1, CD 160, 2B4 and/or TGFR beta): e.g., pidilizumab, AMP-224; PDL1 inhibitors (see e.g., O'Donnell supra): e.g., MSB0010718C; YW243.55.S70, MPDL3280A; MEDI-4736, MSB-0010718C, or MDX-1105; Histone deacetylase inhibitors (HDI, see, e.g., Rahmani et al. Clin. Cancer Res. (2014) 20(18), 4849-4860): e.g. vorinostat; Androgen Receptor inhibitors (see e.g., Thomas et al. Mol. Cancer Ther. (2013) 12(11), 2342-2355): e.g., enzalutamide, abiraterone acetate, orteronel, galeterone, seviteronel, bicalutamide, flutamide; Androgens: e.g., fluoxymesterone; CDK4/6 inhibitors (see, e.g., Gul et al. Am. J. Cancer Res. (2018) 8(12), 2359-2376): e.g., alvocidib, palbociclib, ribociclib, trilaciclib, abemaciclib.

In some embodiments, the one or more additional therapeutic agent is selected from the following agents: anti-FGFR antibodies; FGFR inhibitors, cytotoxic agents; Estrogen Receptor-targeted or other endocrine therapies, immune-checkpoint inhibitors, CDK inhibitors, Receptor Tyrosine Kinase inhibitors, BRAF inhibitors, MEK inhibitors, other PI3K inhibitors, SHP2 inhibitors, and SRC inhibitors. (See Katoh, Nat. Rev. Clin. Oncol. (2019), 16:105-122; Chae, et al. Oncotarget (2017), 8:16052-16074; Formisano et al., Nat. Comm. (2019), 10:1373-1386; and references cited therein.)

The structure of the active compounds identified by code numbers, generic or trade names may be taken from the actual edition of the standard compendium The Merck Index or from databases, e.g., Patents International (e.g., IMS World Publications).

A compound of the current disclosure may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current disclosure can be administered alone or in combination with one or more other therapeutic compounds, possible combination therapy taking the form of fixed combinations or the administration of a compound of the disclosure and one or more other therapeutic compounds being staggered or given independently of one another, or the combined administration of fixed combinations and one or more other therapeutic compounds. A compound of the current disclosure can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this disclosure in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this disclosure. For example, a compound of the present disclosure may be administered with another therapeutic agent simultaneously or sequentially in separate unit dosage forms or together in a single unit dosage form. Accordingly, the present disclosure provides a single unit dosage form comprising a compound of the current disclosure, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. In some embodiments, compositions of this disclosure should be formulated so that a dosage of between 0.01-100 mg/kg body weight/day of an inventive compound can be administered.

In those compositions which comprise an additional therapeutic agent, that additional therapeutic agent and the compound of this disclosure may act synergistically. Therefore, the amount of additional therapeutic agent in such compositions will be less than that required in a monotherapy utilizing only that therapeutic agent. In such compositions, a dosage of between 0.01-1,000 μg/kg body weight/day of the additional therapeutic agent can be administered.

The amount of additional therapeutic agent present in the compositions of this disclosure will be no more than the amount that would normally be administered in a composition comprising that therapeutic agent as the only active agent. In some embodiments, the amount of additional therapeutic agent in the presently disclosed compositions will range from about 50% to 100% of the amount normally present in a composition comprising that agent as the only therapeutically active agent.

The compounds of this disclosure, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this disclosure are another embodiment of the present disclosure.

Any of the compounds and/or compositions of the disclosure may be provided in a kit comprising the compounds and/or compositions. Thus, in some embodiments, the compound and/or composition of the disclosure is provided in a kit.

The disclosure is further described by the following non-limiting Examples.

### EXAMPLES

Examples are provided herein to facilitate a more complete understanding of the disclosure. The following examples serve to illustrate the exemplary modes of making and practicing the subject matter of the disclosure. However, the scope of the disclosure is not to be construed as limited to specific embodiments disclosed in these examples, which are illustrative only.

As depicted in the Examples and General Schemes below, in certain exemplary embodiments, compounds are prepared according to the following general procedures. It will be appreciated that, although the general methods depict the synthesis of certain compounds of the present disclosure, the following general methods, and other methods known to one of ordinary skill in the art, can be applied to other classes and subclasses and species of each of these compounds, as described herein. Additional compounds of the disclosure were prepared by methods substantially similar to those described herein in the Examples and methods known to one skilled in the art.

In the description of the synthetic methods described below, unless otherwise stated, it is to be understood that all reaction conditions (for example, reaction solvent, atmosphere, temperature, duration, and workup procedures) are selected from the standard conditions for that reaction, unless otherwise indicated. The starting materials for the Examples are either commercially available or are readily prepared by standard methods from known materials.

### LIST OF ABBREVIATIONS

aq: aqueous
Ac: acetyl
ACN or MeCN: acetonitrile
AmF: ammonium formate
anhyd.: anhydrous
BINAP: (±)-2,2′-Bis(diphenylphosphino)-1,1′-binaphthalene
Bn: Benzyl
conc.: concentrated
DBU: 1,8-Diazabicyclo[5.4.0]undec-7-ene
DCE: Dichloroethane
DCM: Dichloromethane
DIPEA: Diisopropylamine
DMF: N,N-dimethylformamide
DMP: Dess-Martin periodinane
DMPU: N,N′-Dimethylpropyleneurea
DMSO: dimethylsulfoxide
DIPEA: diisopropylethylamine
EA or EtOAc: ethyl acetate
EDCI, EDC, or EDAC: 1-Ethyl-3-(3-dimethylaminopropyl)carbodiimide
equiv or eq: molar equivalents
Et: ethyl
HATU: 1-[Bis(dimethylamino)methylene]-1H-1,2,3-triazolo[4,5-b]pyridinium 3-oxid
He^{YA}fluorophosphate
HPLC: high pressure liquid chromatography
LCMS or LC-MS: liquid chromatography-mass spectrometry
Ms: methanesulfonyl
NBS: N-bromosuccinimide
NMR: nuclear magnetic resonance
PE: petroleum ether
PMB: p-methoxybenzyl
rt or RT: room temperature
sat: saturated
TBS: tert-butyldimethylsilyl
TEA: triethylamine
Tf: trifluoromethanesulfonate
TFA: trifluoroacetic acid
THF: tetrahydrofuran
TLC: thin layer chromatography
Tol: toluene
UV: ultra violet

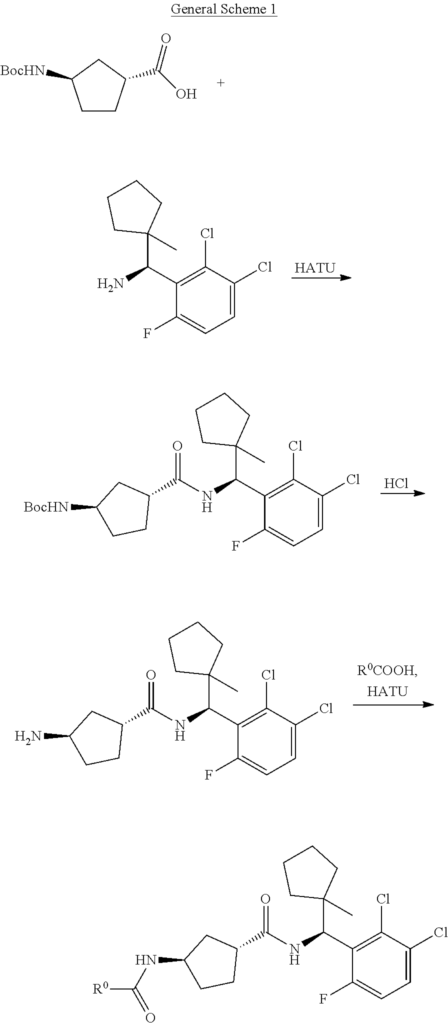

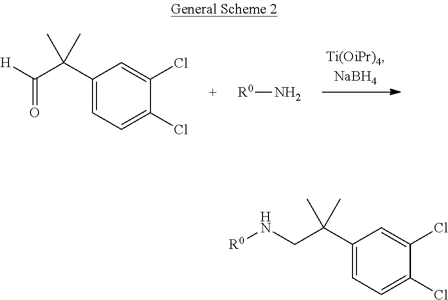

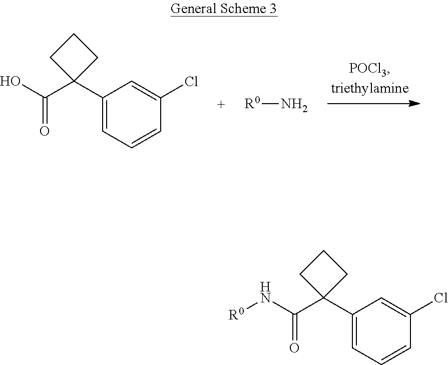

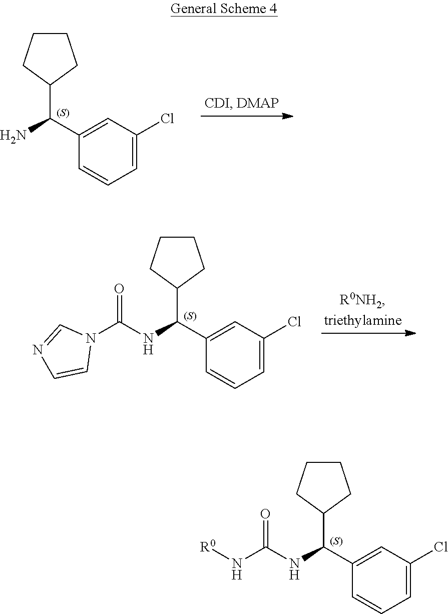

In some examples, compounds of the present disclosure were synthesized in accordance with the exemplary procedures shown in General Schemes 1, 2, 3, or 4. For the purposes of these schemes, R⁰ is an illustrative variable which, when taken together with its contiguous atoms in each instance, represents a commercially available compound, a compound disclosed herein, or other starting materials readily ascertained by one of skill in the art that result in the compounds of the present disclosure. It will be appreciated by one of skill in the art that certain reagents depicted in the General Schemes can be substituted with an appropriate reagent to accomplish an equivalent or substantially similar reaction.

### LC-MS and GC-MS Methods:

Method A: The analytical LC-MS system is equipped with Shimadzu LCMS-2020, PDA detector (operating at 254 nm), ELSD detector, and ESI-source operating in positive ion mode. LC conditions: the column is HALO C18 30*3.0 mm, 2 μm, operating at 40° C. with 1.5 mL/min of a binary gradient consisting of water+0.1% formic acid (A) and acetonitrile+0.1% formic acid (B). The retention time is expressed in minutes based on UV-trace at 254 nm.

| | | |
|---|---|---|
| Gradient: | 0.01 min | 5% B |
| | 1.00 min | 100% B |
| | 1.40 min | 100% B |
| | 1.42 min | 5% B |
| Total run time: | 1.5 min | |

Method B: The analytical LC-MS system is equipped with Shimadzu LCMS-2020, PDA detector (operating at 254 nm), ELSD detector, and ESI-source operating in positive ion mode. LC conditions: the column is Shim-pack Scepter C18-120, 33*3.0 mm, 3 μm, operating at 30° C. with 1.5 mL/min of a binary gradient consisting of water+5 mM NH₄HCO₃ (A) and acetonitrile (B). The retention time is expressed in minutes based on UV-trace at 254 nm.

| | | |
|---|---|---|
| Gradient: | 0.01 min | 10% B |
| | 1.20 min | 95% B |
| | 1.80 min | 95% B |
| | 1.82 min | 10% B |
| Total run time: | 2.0 min | |

Method C: The analytical LC-MS system is equipped with Shimadzu LCMS-2020, PDA detector (operating at 254 nm), ELSD detector, and ESI-source operating in positive ion mode. LC conditions: the column is HALO C18 30*3.0 mm, 2 μm, operating at 40° C. with 1.5 mL/min of a binary gradient consisting of water+0.05% trifluoroacetic acid (A) and acetonitrile+0.05% trifluoroacetic acid (B). The retention time is expressed in minutes based on UV-trace at 254 nm.

| | | |
|---|---|---|
| Gradient: | 0.01 min | 5% B |
| | 1.20 min | 100% B |
| | 1.80 min | 100% B |
| | 1.82 min | 5% B |
| Total run time: | 2.0 min | |

Method D: The analytical LC-MS system is equipped with Shimadzu LCMS-2020, PDA detector (operating at 254 nm), ELSD detector, and ESI-source operating in positive ion mode. LC conditions: the column is Shim-pack Scepter C18-120, 33*3.0 mm, 3 μm, operating at 30° C. with 1.5 mL/min of a binary gradient consisting of water+6.5 mM NH₄HCO₃+ammonia (pH=10) (A) and acetonitrile (B). The retention time is expressed in minutes based on UV-trace at 254 nm.

| | | |
|---|---|---|
| Gradient: | 0.01 min | 10% B |
| | 1.20 min | 95% B |
| | 1.80 min | 95% B |
| | 1.82 min | 10% B |
| Total run time: | 2.0 min | |

Method E: The analytical LC-MS system is equipped with Shimadzu LCMS-2020, PDA detector (operating at 254 nm), ELSD detector, and ESI-source operating in positive ion mode. LC conditions: the column is Kinetex EVO C18 50*3.0 mm, 2.6 μm, operating at 40° C. with 1.2 mL/min of a binary gradient consisting of water+0.04% NH₄OH (A) and acetonitrile (B). The retention time is expressed in minutes based on UV-trace at 254 nm.

| | | |
|---|---|---|
| Gradient: | 0.01 min | 10% B |
| | 1.20 min | 95% B |
| | 1.80 min | 95% B |
| | 1.82 min | 10% B |
| Total run time: | 2.0 min | |

Method F: The analytical LC-MS system is equipped with Shimadzu LCMS-2020, PDA detector (operating at 254 nm), ELSD detector, and ESI-source operating in positive ion mode. LC conditions: the column is Kinetex EVO C18 50*3.0 mm, 2.6 μm, operating at 40° C. with 1.2 mL/min of a binary gradient consisting of water+0.04% NH₄OH (A) and acetonitrile (B). The retention time is expressed in minutes based on UV-trace at 254 nm.

| | | |
|---|---|---|
| Gradient: | 0.01 min | 10% B |
| | 2.00 min | 95% B |
| | 2.60 min | 95% B |
| | 2.70 min | 10% B |
| Total run time: | 2.80 min | |

Method G: The analytical LC-MS system is equipped with Shimadzu LCMS-2020, PDA detector (operating at 254 nm), ELSD detector, and ESI-source operating in positive ion mode. LC conditions: the column is Poroshell HPH-C18 50*3.0 mm, 4 μm, operating at 40° C. with 1.5 mL/min of a binary gradient consisting of water+5 mM NH₄HCO₃ (A) and acetonitrile (B). The retention time is expressed in minutes based on UV-trace at 254 nm.

| | | |
|---|---|---|
| Gradient: | 0.01 min | 10% B |
| | 1.20 min | 95% B |
| | 1.80 min | 95% B |
| | 1.85 min | 10% B |
| Total run time: | 2.0 min | |

Method H: The analytical LC-MS system is equipped with Shimadzu LCMS-2020, PDA detector (operating at 254 nm), ELSD detector, and ESI-source operating in positive ion mode. LC conditions: the column is Shim-Pack-Scepter C18 33*3.0 mm, 3.0 μm, operating at 40° C. with 1.2 mL/min of a binary gradient consisting of water+0.1% Formic acid (A) and acetonitrile+0.07% Formic acid (B). The retention time is expressed in minutes based on UV-trace at 254 nm.

| | | |
|---|---|---|
| Gradient: | 0.01 min | 5% B |
| | 1.30 min | 95% B |
| | 1.75 min | 95% B |
| | 1.80 min | 5% B |
| Total run time: | 1.85 min | |

Method I: The analytical LC-MS system is equipped with Shimadzu LCMS-2020, PDA detector (operating at 220/254 nm), and ESI-source operating in positive ion mode. LC conditions: the column is Kinetex EVO C18 30*2.1 mm, 5 μm, operating at 50° C. with 1.5 mL/min of a binary gradient consisting of water+0.0375% TFA (A) and acetonitrile+0.01875% TFA (B). The retention times (t_{R}) are expressed in minutes based on UV-trace at 254 nm.

| | | |
|---|---|---|
| Gradient: | 0.01 min | 5% B |
| | 0.80 min | 95% B |
| | 1.20 min | 95% B |
| | 1.21 min | 5% B |
| | 1.55 min | 5% B |
| Total run time: | 1.55 min | |

Method J: The analytical LC-MS system is equipped with Shimadzu LCMS-2020, PDA detector (operating at 220/254 nm), and ESI-source operating in positive ion mode. LC conditions: the column is HALO C18 30*3.0 mm, 5 μm, operating at 50° C. with 2.0 mL/min of a binary gradient consisting of water+0.0375% TFA (A) and acetonitrile+0.01875% TFA (B). The retention times (t_{R}) are expressed in minutes based on UV-trace at 254 nm.

| | | |
|---|---|---|
| Gradient: | 0.01 min | 5% B |
| | 0.40 min | 95% B |
| | 0.75 min | 95% B |
| | 0.76 min | 5% B |
| | 1.05 min | 5% B |
| Total run time: | 1.05 min | |

Method K: Column: Waters Acquity UPLC CSH C18, 1.8 μm, 2.1×30 mm at 40° C.; Gradient: 5% to 100% B in 2.0 minutes; hold 100% B for 0.7 minute; run time: 2.7 min; flow 0.9 mL/min; Eluents: A=Milli-Q H₂O+10 mM ammonium formate; pH: 3.8; Eluent B: acetonitrile (no additive); Waters UPLC system equipped with: UV Detector=Waters Acquity PDA (198-360 nm), 20 pts/sec, 220 and 254 nm. MS Detector Waters SQD, ESI (ES+/ES−, 120-1200 amu).
Method L: HPLC-MS method: Waters Alliance UPLC CSH C18, 3.5 μm, 4.6×30 mm at 40° C.; 5% B for 0.2 min, 5% to 100% B in 1.8 minutes; hold 100% B for 1 minute, run time=3.0 min, flow 3 mL/min; Eluents: A=Milli-Q H₂O+10 mM ammonium formate pH=3.8; B=acetonitrile. Waters Alliance HPLC system. UV Detector=Waters 2996 PDA, 198-360 nm. MS Detector=Waters ZQ 2000.
Method M: HPLC-MS method: Waters Alliance UPLC CSH C18, 3.5 μm, 4.6×30 mm at 40° C.; 5% B for 0.5 min, 5% to 100% B in 5.0 minutes; hold 100% B for 0.7 minute, 100% B for 1.5 min, run time=7.0 min, flow 3 mL/min; Eluents: A=Milli-Q H₂O+10 mM ammonium formate, pH 3.8; B=MeCN. Waters Alliance HPLC system. UV Detector=Waters 2996 PDA, 198-360 nm. MS Detector=Waters ZQ 2000.
GCMS method (method Z): The GC-MS system consists of Agilent GCMS 7890B and Detector Channel FID.

### The MS Detector of Acquisition Mode:

Start Time: 2.00 min; End Time: 11.75 min; Acquisition Mode: Scan; Interface Type: EI
Threshold: 150; Scan Speed: 1562; Start m/z: 50.00; End m/z: 600.00; MS Source: 230.00° C.; MS Quad: 150.00° C.; Solvent Cut Time: 2.00 min.

### GC Parameters:

Column: HP-5MS, 30 m×0.25 mm×0.25 μm; Column Oven Temp: 50.0° C.; injection volume: 1 μL;
Column Flow: 1.0 ml/min; Injection temperature: 300° C.; Injection Mode: Split; Split Ratio: 20:1;
Detector temperature: 300° C.; Initial temperature: 50° C. for 0.5 min then 40° C./min to 300° C. for 11.75 min.
Makeup Gas: He; Makeup Flow: 25.0 mL/min; H₂; Flow: 30.0 mL/min; Air Flow: 400.0 mL/min;
Final temperature: 325° C.

### Example 1

### (S)-(2,3-dichloro-6-fluorophenyl)(1-methylcyclopentyl)methanamine

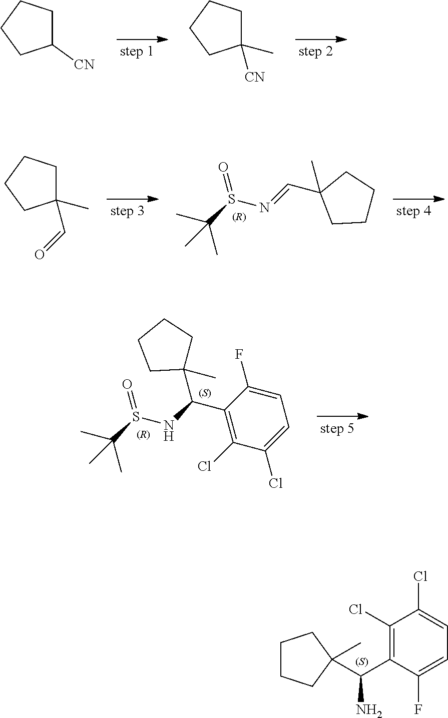

### Step 1. Synthesis of 1-methylcyclopentane-1-carbonitrile

To a solution of LiHMDS (1.00 M, 1.50 L) was a solution of cyclopentanecarbonitrile (130 g, 1.37 mol) in THF (650 mL) added dropwise at −60° C. under N₂. After the addition, the reaction mixture was stirred at −60° C. for 1 hour. Then Mel (111 mL, 1.78 mol) was added dropwise at −60° C. The reaction mixture was allowed to warm to 20° C. and stirred at 20° C. for 12 hours. The mixture was poured into saturated aqueous NH₄Cl solution (2.00 L) and extracted with ethyl acetate (1.50 L*2). The combined organic layer was washed with 1N HCl (1.00 L) and brine (1.50 L*2), dried over Na₂SO₄, filtered and concentrated under reduced pressure to give 1-methylcyclopentane-1-carbonitrile (150 g, crude) as a yellow oil. ¹H NMR: (400 MHz, CDCl₃) δ 2.16-2.14 (m, 2H), 1.85-1.77 (m, 4H), 1.63-1.59 (m, 2H), 1.41 (s, 3H).

### Step 2. Synthesis of 1-methylcyclopentane-1-carbaldehyde

To a solution of DIBAL-H (1.00 M in THF, 1.51 L) was added dropwise a solution of 1-methylcyclopentane-1-carbonitrile (150 g, 1.37 mol) in DCM (150 mL) at −65° C. under N₂. The mixture was stirred at −65° C. for 1 hour and poured into saturated aqueous NH₄Cl solution (5.00 L) under stirring. The pH was adjusted to -3 with HCl (6 N, 1.20 L), then extracted with DCM (2.00 L*2). The combined organic layer was washed with brine (1.50 L*2), dried over Na₂SO₄, filtered and concentrated (15° C.) under reduced pressure to give 1-methylcyclopentane-1-carbaldehyde (130 g, crude) as a colorless liquid. The crude product was used in the next step without purification.

### Step 3. Synthesis of (R)-2-methyl-N-((1-methylcyclopentyl)methylene)propane-2-sulfinamide

To a mixture of 1-methylcyclopentane-1-carbaldehyde (120 g, 1.07 mol) in THF (600 mL) was added (R)-2-methylpropane-2-sulfinamide (156 g, 1.28 mol), Ti(OⁱPr)₄ (608 g, 2.14 mol) at 20° C. The mixture was heated to 75° C. and stirred at 75° C. for 2 hours. The mixture was poured into brine (4.00 L). The mixture was filtered, and the filtrate was washed with ethyl acetate (1.50 L*3). The filtrate was washed with brine (2.00 L) and concentrated to give a residue. The residue was purified by column chromatography (SiO₂, petroleum ether/ethyl acetate=10/1 to 5/1) to give (R)-2-methyl-N-((1-methylcyclopentyl)methylene)propane-2-sulfinamide (120 g, 557 mmol) as a yellow oil. ¹H NMR: (400 MHz, CDCl₃) δ 7.95 (s, 1H), 1.94-1.92 (m, 2H), 1.75-1.68 (m, 4H), 1.50-1.46 (m, 2H), 1.22 (s, 3H), 1.19 (s, 9H).

### Step 4. Synthesis of (R)—N—((S)-(2,3-dichloro-6-fluorophenyl)(1-methylcyclopentyl)methyl)-2-methylpropane-2-sulfinamide

To a mixture of 1,2-dichloro-4-fluorobenzene (99.6 g, 604 mmol) in THF (1.00 L) was added dropwise n-BuLi (2.50 M in hexanes, 241 mL) at −65° C. under N₂, then the mixture was stirred at −65° C. for 0.5 hour. To the mixture was added (R)-2-methyl-N-((1-methylcyclopentyl)methylene)propane-2-sulfinamide (100 g, 464 mmol) in THF (100 mL). It was stirred at −65° C. for 1 hour. The mixture was poured into saturated aqueous NH₄Cl solution (10%, 2.00 L) and extracted with ethyl acetate (1.00 L*2). The combined organic layers were washed with brine (1.00 L*2) and concentrated under reduced pressure to give a residue. The residue was purified by preparative HPLC (column: Phenomenex luna C18 250 mm*100 mm, 10 μm; mobile phase A: water (formic acid) B: acetonitrile; B: 60%-80% over 25 min) to give (R)—N—((S)-(2,3-dichloro-6-fluorophenyl)(1-methylcyclopentyl)methyl)-2-methylpropane-2-sulfinamide (120 g, 316 mmol) as a yellow oil. ¹H NMR: (400 MHz, DMSO-d₆) δ 7.66-7.62 (m, 1H), 7.32-7.27 (m, 1H), 5.13 (d, J=8.4 Hz, 1H), 4.91 (d, J=8.4 Hz, 1H), 1.74-1.62 (m, 6H), 1.39-1.36 (m, 1H), 1.26-1.22 (m, 1H), 0.97-0.95 (m, 12H).

### Step 5. Synthesis of (S)-(2,3-dichloro-6-fluorophenyl)(1-methylcyclopentyl)methanamine

To a mixture of (R)—N—((S)-(2,3-dichloro-6-fluorophenyl)(1-methylcyclopentyl)methyl)-2-methylpropane-2-sulfinamide (110 g, 289 mmol) in ethyl acetate (1.10 L) was added HCl in EtOAc (4 M, 275 mL). The mixture was stirred at 20° C. for 1 hour. The mixture was concentrated. To the residue was added H₂O (1.50 L), and it was washed with ethyl acetate (1.00 L*2). To the aqueous layer was added saturated aqueous NaHCO₃ solution (800 mL) until pH=9. The mixture was extracted with ethyl acetate (1.00 L*2) and the combined organic layers were washed with brine (500 mL*2), dried over Na₂SO₄, filtered and concentrated under reduced pressure to give (S)-(2,3-dichloro-6-fluorophenyl)(1-methylcyclopentyl)methanamine (52.23 g, 188 mmol) as a yellow oil. ¹H NMR: (400 MHz, DMSO-d₆) δ 7.59-7.56 (m, 1H), 7.27-7.22 (m, 1H), 4.32 (s, 1H), 2.10 (s, 2H), 1.79-1.59 (m, 6H), 1.29-1.26 (m, 1H), 1.14-1.11 (m, 1H), 0.86 (d, J=2.4 Hz, 3H).

### Example 2

### (2r,4r)-6,8-dioxo-5,7-diazaspiro[3.4]octane-2-carboxylic acid

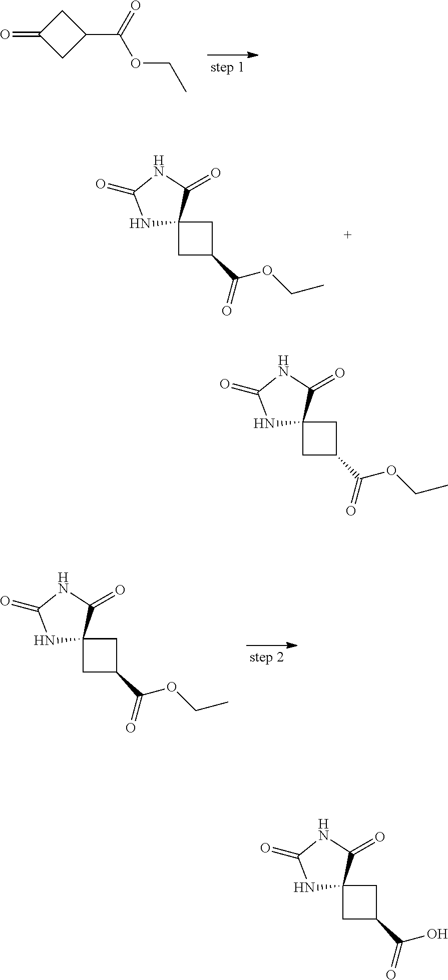

### Step 1. Synthesis of Ethyl 6,8-dioxo-5,7-diazaspiro[3.4]octane-2-carboxylate

To a mixture of ethyl 3-oxocyclobutane-1-carboxylate (113 g, 791 mmol), (NH₄)₂CO₃ (114 g, 1.19 mol) in EtOH (1.50 L) and H₂O (500 mL) was added NaCN (38.8 g, 791 mmol) at 20° C. The mixture was heated to 35° C. and stirred at 35° C. for 12 hours. Four such batches were combined. The reaction mixture was extracted with ethyl acetate (2.00 L*3). The combined organic layers were dried over anhydrous Na₂SO₄, filtered and concentrated to give a white solid. It was purified by preparative HPLC (column: Phenomenex luna C18 250*50 mm*10 μm; mobile phase A: water (0.1% TFA) B: acetonitrile; gradient: B % 10%-35% over 21 minutes). The eluent was concentrated to remove most of acetonitrile and extracted with ethyl acetate (5.00 L*6). The combined organic layer was dried over anhydrous sodium sulfate, filtered and concentrated under vacuum. Ethyl (2r,4r)-6,8-dioxo-5,7-diazaspiro[3.4]octane-2-carboxylate (90.0 g, 411 mmol) was obtained as a white solid. ¹H NMR: (400 MHz, DMSO-d6) δ 10.6 (s, 1H), 8.48 (s, 1H), 8.46 (s, 1H), 4.12-4.05 (m, 2H), 3.25-3.18 (m, 1H), 2.69-2.65 (m, 2H), 2.41-2.34 (m, 2H), 1.19 (t, J=6.8 Hz, 3H).

Ethyl (2s,4s)-6,8-dioxo-5,7-diazaspiro[3.4]octane-2-carboxylate (150 g, 66% purity, containing 30% ethyl (2r,4r)-6,8-dioxo-5,7-diazaspiro[3.4]octane-2-carboxylate) was also obtained.

### Step 2. Synthesis of (2r,4r)-6,8-dioxo-5,7-diazaspiro[3.4]octane-2-carboxylic acid

To a solution of ethyl (2r,4r)-6,8-dioxo-5,7-diazaspiro[3.4]octane-2-carboxylate (90.0 g, 424 mmol) in MeOH (450 mL) and H₂O (200 mL) was added LiOH H₂O (44.5 g, 1.06 mol) at 20° C. The mixture was stirred at 20° C. for 1 hour. The mixture was adjusted to pH=1˜2 with 3 N HCl. The precipitate was collected by filtration. The filter cake was triturated with ethyl acetate (300 mL) at 25° C. for 2 hours and filtered. The filter cake was dried over vacuum to afford (2r,4r)-6,8-dioxo-5,7-diazaspiro[3.4]octane-2-carboxylic acid (52.0 g, 279 mmol) as a white solid. ¹H NMR: (400 MHz, DMSO-d₆) δ 12.33 (s, 1H), 10.60 (s, 1H), 8.48 (s, 1H), 3.16-3.13 (m, 1H), 2.70-2.64 (m, 2H), 2.35-2.29 (m, 2H).

### Example 3

### (2r,4S)—N—((S)-(2,3-dichloro-6-fluorophenyl)(1-methylcyclopentyl)methyl)-6,8-dioxo-5,7-diazaspiro[3.4]octane-2-carboxamide

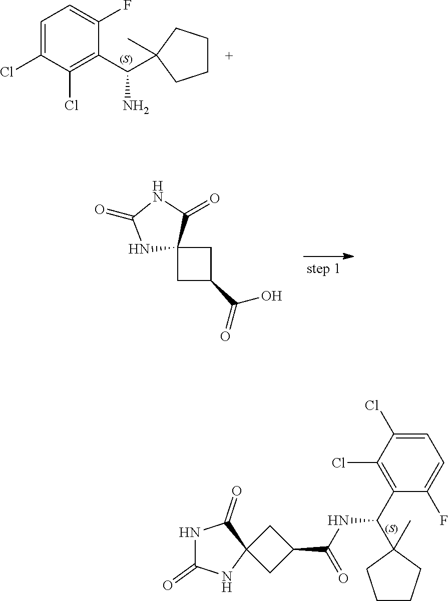

### Step 1. Synthesis of (2r,4S)—N—((S)-(2,3-dichloro-6-fluorophenyl)(1-methylcyclopentyl)methyl)-6,8-dioxo-5,7-diazaspiro[3.4]octane-2-carboxamide

A mixture of (S)-(2,3-dichloro-6-fluorophenyl)(1-methylcyclopentyl)methanamine (40 mg, 0.14 mmol), (2r,4r)-6,8-dioxo-5,7-diazaspiro[3.4]octane-2-carboxylic acid (27 mg, 0.14 mmol), TEA (44 mg, 0.43 mmol) and T₃P (0.14 g, 50% wt, 0.22 mmol) in DMF (1 mL) was stirred at 25° C. for 1 h. The reaction was quenched with water (5 ml) and extracted with ethyl acetate (10 ml*3). The combined organic layers were washed with brine (5 ml). The mixture was dried over Na₂SO₄ and concentrated. The residue was purified by C18 flash chromatography (CH₃CN/water, 25%-60% CH₃CN over 20 min) to afford (2r,4S)—N—((S)-(2,3-dichloro-6-fluorophenyl)(1-methylcyclopentyl)methyl)-6,8-dioxo-5,7-diazaspiro[3.4]octane-2-carboxamide (43.1 mg, 97.4 μmol) as a white solid. ¹H NMR (400 MHz, DMSO-d6) δ 10.57 (s, 1H), 8.62 (s, 1H), 8.23 (d, J=8.6 Hz, 1H), 7.61 (dd, J=8.9, 5.0 Hz, 1H), 7.25 (dd, J=10.8, 9.0 Hz, 1H), 5.50 (d, J=8.5 Hz, 1H), 3.30-3.26 (m, 1H), 2.71-2.53 (m, 2H), 2.21 (dd, J=24.5, 11.2 Hz, 2H), 1.59 (s, 6H), 1.37 (s, 1H), 1.27 (s, 1H), 0.96 (d, J=2.8 Hz, 3H). LC MS RT 0.928 min, [M+H]⁺ 442, LCMS method C.

### Example 4

### (S)-(3-chlorophenyl)(cyclopentyl)methanamine

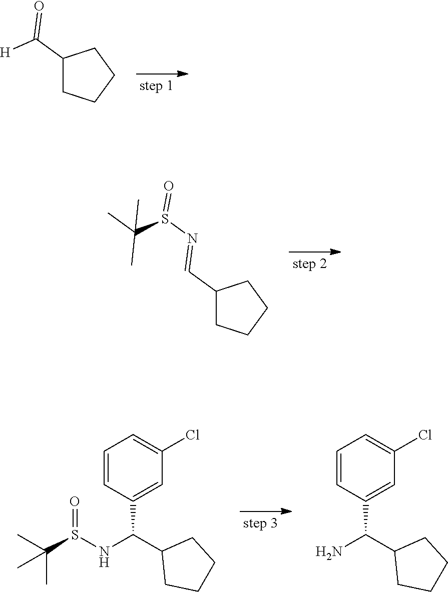 [SMILES: O=CC1CCCC1 ; CC(C)(C)[S@@](=O)/N=C/C1CCCC1 ; CC(C)(C)[S@@](=O)N[C@H](c1cccc(Cl)c1)C1CCCC1 ; N[C@H](c1cccc(Cl)c1)C1CCCC1]

### Step 1. Synthesis of (R)—N-(cyclopentylmethylene)-2-methylpropane-2-sulfinamide

To a solution of cyclopentanecarbaldehyde (112 g, 1.15 mol) and (R)-2-methylpropane-2-sulfinamide (167 g, 1.38 mol) in THF (560 mL) was added Ti(OⁱPr)₄ (651 g, 2.29 mol) under N₂ atmosphere at 25° C. The mixture was heated to 75° C. and stirred at 75° C. for 2 hours. Two batches were carried out in parallel and combined in the workup. After cooling to room temperature, to the mixture was added brine (3.00 L). The suspension was filtered. The filter cake was washed with ethyl acetate (5.00 L*2). The organic phase was separated and the aqueous phase was extracted with ethyl acetate (3.00 L). The combined organic phase was washed with brine (3.00 L), dried over Na₂SO₄, filtered and concentrated. The residue was purified by column chromatography (SiO₂, petroleum ether:ethyl acetate 1:0 to 10:1). (R)—N-(cyclopentylmethylene)-2-methylpropane-2-sulfinamide (357 g, 1.77 mol) was obtained as a yellow oil. ¹H NMR: (400 MHz, CDCl₃) δ 8.00 (d, J=5.6 Hz, 1H), 2.98-2.94 (m, 1H), 1.94-1.83 (m, 2H), 1.77-1.62 (m, 6H), 1.19 (s, 9H)

### Step 2. Synthesis of (R)—N—((S)-(3-chlorophenyl)(cyclopentyl)methyl)-2-methylpropane-2-sulfinamide

Two batches were carried out in parallel and combined in the workup. To a solution of (R)—N-(cyclopentylmethylene)-2-methylpropane-2-sulfinamide (160 g, 795 mmol) and 1-bromo-3-chlorobenzene (228 g, 1.19 mol) in THF (800 mL) was added n-BuLi (2.50 M in hexanes, 477 mL) dropwise at −70˜−60° C. under N₂. The reaction was stirred at −70˜−60° C. for 2 hours.

The mixture was poured into saturated NH₄Cl solution (5.00 L) and extracted with ethyl acetate (2.00 L*3). The combined organic phase was washed with brine (2.00 L), dried over Na₂SO₄, filtered and concentrated to give a yellow oil (563 g). The crude product was used in the next step without purification. LCMS: RT 1.030 min, [M+H]⁺ 314.1, LCMS method I.

### Step 3. Synthesis of (S)-(3-chlorophenyl)(cyclopentyl)methanamine

Two equal batches were carried out in parallel. To a solution of (R)—N—((S)-(3-chlorophenyl)(cyclopentyl)methyl)-2-methylpropane-2-sulfinamide (264 g, 757 mmol) in ethyl acetate (2.60 L) was added HCl in EtOAc (4.00 M, 473 mL) at 25° C. The mixture was stirred at 25° C. for 1 hour. A large amount of white solid was formed. The two batches of reaction mixture were combined. The suspension was concentrated to 4.0 L and the suspension was filtered. The filter cake was washed with ethyl acetate (200 mL*2). The filter cake was partitioned between ethyl acetate (2.00 L) and saturated NaHCO₃ solution (2.50 L). The suspension was stirred for 10 minutes until the solid disappeared. The organic phase was separated and the aqueous phase was extracted with ethyl acetate (1.00 L*2). The combined organic phase was washed with brine (2.00 L), dried over Na₂SO₄, filtered and concentrated to give (S)-(3-chlorophenyl)(cyclopentyl)methanamine (220 g, crude) as a yellow oil. To a solution of (2R,3R)-2,3-dihydroxysuccinic acid (80.0 g, 535 mmol) in MeOH (1.30 L) was added crude (S)-(3-chlorophenyl)(cyclopentyl)methanamine (110 g, 525 mmol) at 25° C. The reaction was stirred at 25° C. for 10 minutes, and a white solid was formed. The reaction mixture was set aside for 3 hours. The suspension was filtered. The filter cake was washed with methanol (100 mL*2), dried under vacuum to give a solid (260 g, ee %=98.0%). The product was diluted with methanol (1.00 L) and heated at 80° C. for 1.0 hour until the solid fully dissolved. The reaction was set aside for 72 hours. A white solid precipitated. The reaction mixture was filtered and the filter cake was washed with methanol (100 mL*2). The filter cake was partitioned between saturated aq. NaHCO₃ (2.50 L) and ethyl acetate (2.00 L). The organic phase was separated. The aqueous phase was extracted with ethyl acetate:methanol=10:1 (2.00 L*2). The combined organic layers were dried over Na₂SO₄, filtered and concentrated under reduced pressure to give (S)-(3-chlorophenyl)(cyclopentyl)methanamine (94.0 g, ee %=100%). ¹H NMR: (400 MHz DMSO-d₆) δ 7.40 (s, 1H), 7.32-7.22 (m, 3H), 3.54 (d J=8.4 Hz, 1H), 2.18 (s, 2H), 2.00-1.90 (m, 1H), 1.79-1.71 (m, 1H), 1.60-1.45 (m, 3H), 1.42-1.30 (m, 2H), 1.25-1.17 (m, 1H), 1.11-1.02 (m, 1H).

### Example 5

### (S)-(3-chloro-2,6-difluorophenyl)(4-fluorobicyclo[2.2.1]heptan-1-yl)methanamine hydrochloride

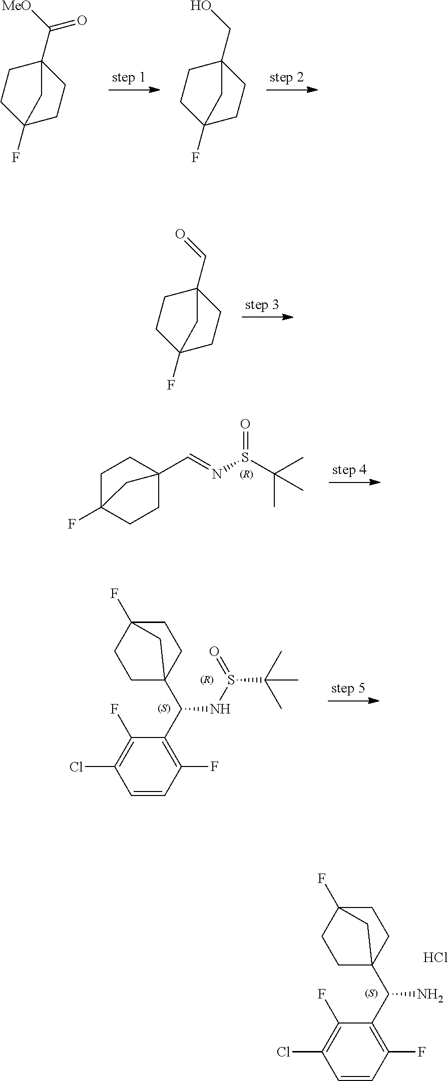

### Step 1. Synthesis of (4-fluorobicyclo[2.2.1]heptan-1-yl)methanol

To a solution of methyl 4-fluorobicyclo[2.2.1]heptane-1-carboxylate (165 g, 958 mmol) in THF (1.65 L) was added dropwise LiAlH₄ (2.5 M in THF, 460 mL) at 0˜10° C. The mixture was warmed to 20° C. and stirred at 20° C. for 2 hours. The reaction mixture was slowly poured into 1 M aqueous HCl (5.00 L) and extracted with ethyl acetate (5.00 L*2). The organic phase was washed with brine (5.00 L), dried over anhydrous Na₂SO₄, filtered and concentrated under vacuum to give (4-fluorobicyclo[2.2.1]heptan-1-yl)methanol (146 g, crude) as a light yellow oil. ¹H NMR (400 MHz, CDCl₃) δ 3.61 (s, 2H), 2.05-1.93 (m, 2H), 1.85-1.72 (m, 4H), 1.52-1.39 (m, 4H).

### Step 2. Synthesis of 4-fluorobicyclo[2.2.1]heptane-1-carbaldehyde

To a solution of (4-fluorobicyclo[2.2.1]heptan-1-yl)methanol (150 g, 1.04 mol) in DCM (1.13 L) was added DMSO (244 mL, 3.12 mol), TEA (724 mL, 5.20 mol) at 20° C. The mixture was cooled to 0˜5° C. SO₃ Py (745 g, 4.68 mol) was added to the mixture at 0˜5° C. The mixture was warmed to 20° C. and stirred at 20° C. for 2 hours. The mixture was poured into water (5.00 L) and extracted with DCM (5.00 L*2). The organic phase was dried over anhydrous Na₂SO₄, filtered and concentrated under vacuum at 25° C. The residue was dissolved in ethyl acetate (2.00 L). The organic phase was washed with 1 N aqueous HCl (1.50 L*2), brine (1.50 L), dried over anhydrous Na₂SO₄, filtered and concentrated under vacuum to give 4-fluorobicyclo[2.2.1]heptane-1-carbaldehyde (112 g, crude) as a yellow oil. ¹H NMR (400 MHz, CDCl₃) δ 9.72 (s, 1H), 2.21-2.10 (m, 2H), 2.04-1.95 (m, 2H), 1.90-1.82 (m, 4H), 1.65-1.58 (m, 2H).

### Step 3. Synthesis of (R)—N-((4-fluorobicyclo[2.2.1]heptan-1-yl)methylene)-2-methylpropane-2-sulfinamide

To a solution of 4-fluorobicyclo[2.2.1]heptane-1-carbaldehyde (110 g, 774 mmol), (R)-2-methylpropane-2-sulfinamide (93.8 g, 774 mmol) in THF (1.10 L) was added, followed by Ti(OⁱPr)₄ (440 g, 1.55 mol) at 25° C. The mixture was heated to 75° C. and stirred at 75° C. for 2 hours. The mixture was cooled to 25° C., diluted with ethyl acetate (4.00 L) and poured into water (4.00 L). The mixture was filtered. The filtrate was separated, and the organic phase was washed with brine (3.00 L), dried over anhydrous Na₂SO₄, filtered and concentrated under vacuum. The residue was combined with another batch (which started with 20.0 g of the aldehyde) and purified by column chromatography (SiO₂, petroleum ether:ethyl acetate 1:0 to 10:1) to give (R)—N—((S)-(3-chloro-2,6-difluorophenyl)(4-fluorobicyclo[2.2.1]heptan-1-yl)methyl)-2-methylpropane-2-sulfinamide (150 g, 599 mmol) as a white solid. ¹H NMR (400 MHz, CDCl₃) δ 8.09 (s, 1H), 2.13-1.94 (m, 4H), 1.91-1.80 (m, 4H), 1.72-1.63 (m, 2H), 1.19 (s, 9H); ¹⁹F NMR (376 MHz, CDCl₃) δ −176.81

### Step 4. Synthesis of (R)—N—((S)-(3-chloro-2,6-difluorophenyl)(4-fluorobicyclo[2.2.1]heptan-1-yl)methyl)-2-methylpropane-2-sulfinamide

To a solution of 1-chloro-2,4-difluorobenzene (8.97 g, 60.4 mmol) in THF (114 mL) was added n-BuLi (2.50 M in hexanes, 24.1 mL) at −65° C. The mixture was stirred at −65° C. for 0.5 hour, then a solution of (R)—N-((4-fluorobicyclo[2.2.1]heptan-1-yl)methylene)-2-methylpropane-2-sulfinamide (11.4 g, 46.4 mmol) in THF (114 mL) was added at −65° C. The mixture was stirred at −65° C. for 2 hours. The reaction mixture was poured into saturated NH₄Cl solution (500 mL). The aqueous phase was extracted with ethyl acetate (300 mL*2). The combined organic phase was washed with saturated brine (500 mL*2), dried over anhydrous Na₂SO₄, filtered and concentrated in vacuum to give (R)—N—((S)-(3-chloro-2,6-difluorophenyl)(4-fluorobicyclo[2.2.1]heptan-1-yl)methyl)-2-methylpropane-2-sulfinamide (20.0 g, crude) as a yellow solid. LCMS RT 0.598 min, [M+H]⁺ 394.1, LCMS method J.

### Step 5. Synthesis of (S)-(3-chloro-2,6-difluorophenyl)(4-fluorobicyclo[2.2.1]heptan-1-yl)methanamine hydrochloride

To a solution of (R)—N—((S)-(3-chloro-2,6-difluorophenyl)(4-fluorobicyclo[2.2.1]heptan-1-yl)methyl)-2-methylpropane-2-sulfinamide (20.0 g, 50.7 mmol) in MeOH (100 mL) was added HCl (4.00 N in MeOH, 100 mL) at 25° C. The mixture was stirred at 25° C. for 1 hour. The reaction mixture was concentrated in vacuum to give the crude product. The residue was triturated with ethyl acetate (100 mL) at 20° C. for 30 minutes, filtered and the filter cake was dried under vacuum at 50° C. to give (S)-(3-chloro-2,6-difluorophenyl)(4-fluorobicyclo[2.2.1]heptan-1-yl)methanamine hydrochloride (10.0 g, 30.6 mmol) as a white solid. LCMS: RT 0.423 min, [M+H]⁺ 290.1, LCMS method J; ¹H NMR (400 MHz, MeOH-d4) δ 7.69-7.64 (m, 1H), 7.22-7.17 (m, 1H), 3.32-3.31 (m, 1H), 1.99-1.81 (m, 8H), 1.77-1.57 (m, 2H).

### Example 6

### (1S,3S,4R)-3-((tert-butoxycarbonyl)amino)-4-hydroxycyclopentane-1-carboxylic acid

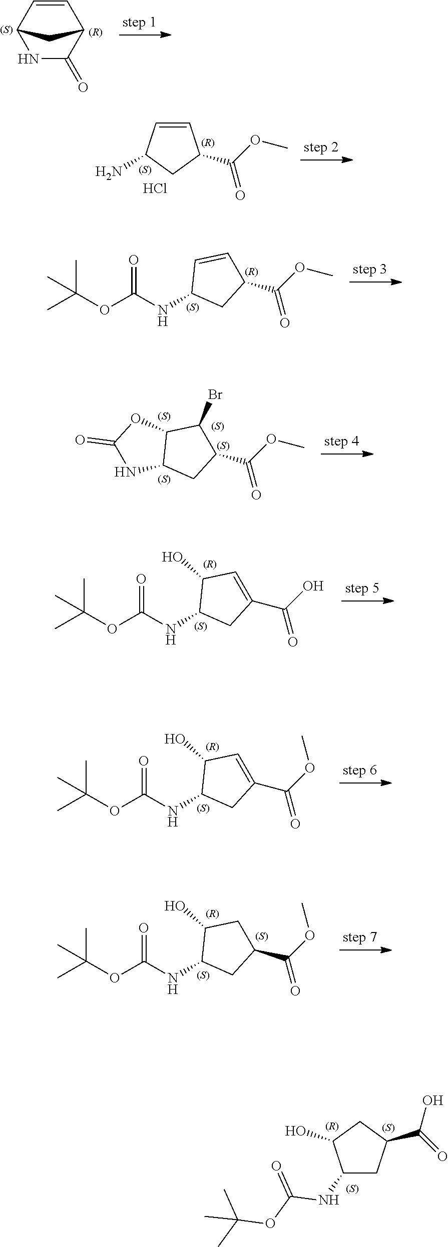

### Step 1. Synthesis of methyl (1R,4S)-4-aminocyclopent-2-ene-1-carboxylate hydrochloride

To a solution of (1S,4R)-2-azabicyclo[2.2.1]hept-5-en-3-one (535 g, 4.90 mol) in MeOH (1605 mL) was added SOCl₂ (213 mL, 2.94 mol) at 0° C. and the solution was stirred at 0° C. for 2 hours. The solution was concentrated under vacuum. The crude product was triturated with MTBE (1000 mL) at 20° C. for 30 minutes to give methyl (1R,4S)-4-aminocyclopent-2-ene-1-carboxylate hydrochloride (860 g, 4.84 mol). ¹HNMR: (400 MHz DMSO-d6) δ 8.37 (s, 3H), 6.05-6.07 (m, 1H), 5.87-5.89 (m, 1H), 4.16 (s, 1H), 3.68-3.70 (m, 1H), 3.64 (m, 3H), 2.53-2.59 (m, 1H), 1.90-1.97 (m, 1H).

### Step 2. Synthesis of methyl (1R,4S)-4-((tert-butoxycarbonyl)amino)cyclopent-2-ene-1-carboxylate

To a solution of methyl (1R,4S)-4-aminocyclopent-2-ene-1-carboxylate hydrochloride (750 g, 4.22 mol) and Boc₂O (919 g, 4.22 mol) in DCM (4.5 L) was added TEA (728 mL, 4.22 mol) at 0° C. and the solution was stirred at 25° C. for 12 hours. The reaction was quenched by water (1000 mL) and extracted with dichloromethane (500 mL*2). The combined organic layers were washed with brine (500 mL), dried over sodium sulfate, filtered and concentrated under reduced pressure to give methyl (1R,4S)-4-((tert-butoxycarbonyl)amino)cyclopent-2-ene-1-carboxylate (1.00 kg, 4.13 mol). ¹HNMR: (400 MHz CDCl₃) δ 5.83-5.87 (m, 1H), 4.88 (s, 1H), 4.77 (s, 1H), 3.69 (s, 3H), 3.45-3.47 (m, 1H), 2.45-2.53 (m, 1H), 1.83-1.87 (m, 1H), 1.25 (s, 9H).

### Step 3. Synthesis of methyl (3aS,5S,6S,6aS)-6-bromo-2-oxohexahydro-2H-cyclopenta[d]oxazole-5-carboxylate

To a solution of methyl (1R,4S)-4-((tert-butoxycarbonyl)amino)cyclopent-2-enc-1-carboxylate (630 g, 2.61 mol) in THF (2.0 L) and H₂O (189 mL) was added NBS (511 g, 2.87 mol) at 0° C. and the solution was stirred at 25° C. for 12 hours. The reaction was concentrated under reduced pressure. The residue was dissolved in dichloromethane (2000 mL) and washed sequentially with HCl (500 mL, 1 M), saturated Na₂SO₃ (aq., 1000 mL) and brine (500 mL) before drying over MgSO₄. The organic phase was concentrated under reduced pressure to give methyl (3aS,5S,6S,6aS)-6-bromo-2-oxohexahydro-2H-cyclopenta[d]oxazole-5-carboxylate (854 g, 3.23 mol) as a white solid. ¹HNMR: (400 MHz CDCl₃) δ 6.27 (s, 1H), 5.13-5.15 (d, J=8 Hz, 1H), 4.76 (s, 1H), 4.40-4.43 (m, 1H), 3.74 (s, 1H), 3.19-3.23 (m, 1H), 2.23-2.54 (m, 2H).

### Step 4. Synthesis of (3R,4S)-4-((tert-butoxycarbonyl)amino)-3-hydroxycyclopent-1-ene-1-carboxylic acid

Two reactions were run in parallel. To a solution of methyl (3aS,5S,6S,6aS)-6-bromo-2-oxohexahydro-2H-cyclopenta[d]oxazole-5-carboxylate (375 g, 1.42 mol) in H₂O (1.6 L) and MeOH (1.6 L) was added KOH (318 g, 5.68 mol) at 0° C. and the solution was stirred at 90° C. for 12 hours. The above solution was concentrated and dissolved in THF (700 mL) and Boc₂O (309 g, 1.42 mol) was added. The solution was stirred at 20° C. for 4 h. The two reactions were combined for work up. The resulting mixture was concentrated in vacuum and then ethyl acetate (500 mL) and H₂O (400 mL) were added. The aqueous phase was separated and the pH was adjusted to 3 with HCl (1 M). The solution was extracted with ethyl acetate (1000 mL*2). The combined organic layers were washed with brine (500 mL), dried over sodium sulfate, filtered and concentrated under reduced pressure to give (3R,4S)-4-((tert-butoxycarbonyl)amino)-3-hydroxycyclopent-1-ene-1-carboxylic acid (600 g, 2.47 mol) as a white solid. ¹HNMR (400 MHz DMSO-d6) δ 12.49 (s, 1H), 6.51 (s, 1H), 6.31-6.33 (m, 1H), 5.03 (s, 1H), 4.50 (s, 1H), 3.97-4.00 (m, 1H), 2.55-2.61 (m, 1H), 2.31-2.37 (m, 1H), 1.45 9 s, 9H).

### Step 5. Synthesis of methyl (3R,4S)-4-((tert-butoxycarbonyl)amino)-3-hydroxycyclopent-1-ene-1-carboxylate

To a solution of (3R,4S)-4-((tert-butoxycarbonyl)amino)-3-hydroxycyclopent-1-ene-1-carboxylic acid (400 g, 1.64 mol) in MeOH (2.8 L) was added TEA (389 mL, 2.80 mol). The mixture was cooled to 0° C. and methyl chloroformate (216 mL, 2.80 mol) was added dropwise. The mixture was stirred at 0° C. for 1 hour, then stirred at 15° C. for 12 hours. The reaction mixture was concentrated under reduced pressure. The residue was diluted with DCM (1000 mL). The organic layer was washed with 1 M potassium hydrogen sulfate (aq) (500 ml*2), saturated NaHCO₃ solution (500 ml*2), dried over Na₂SO₄, filtered and concentrated under reduced pressure to give methyl (3R,4S)-4-((tert-butoxycarbonyl)amino)-3-hydroxycyclopent-1-ene-1-carboxylate (300 g, 1.10 mol) as a white solid. ¹HNMR: (400 MHz CDCl₃) δ 6.69 (s, 1H), 5.134 (d, J=8 Hz, 1H), 4.76 (s, 1H), 4.24 (s, 1H), 3.75 (s, 3H), 2.88-2.94 (m, 1H), 2.03-2.51 (m, 2H), 1.26 (s, 9H).

### Step 6. Synthesis of methyl (1S,3S,4R)-3-((tert-butoxycarbonyl)amino)-4-hydroxycyclopentane-1-carboxylate

Three reactions were run in parallel. [(S,S)-(Me-DuPHOS)-Rh(COD)]BF₄ (2.36 g, 3.89 mmol) was added to a degassed solution of methyl (3R,4S)-4-((tert-butoxycarbonyl)amino)-3-hydroxycyclopent-1-ene-1-carboxylate (50 g, 194 mmol) in MeOH (250 mL). The reaction mixture was transferred to a hydrogenation bomb and after purging with N₂ and then H₂, an H₂ pressure of 2 MPa was applied and the reaction was stirred for 14 hours at 25° C. The pressure was released and the bomb was purged with N₂. Concentration of the reaction mixture gave a residue which was dissolved in DCM (500 mL). Addition of silica (150 g) with stirring removed the catalyst from the reaction and filtration and concentration of the organic solution gave methyl (1S,3S,4R)-3-((tert-butoxycarbonyl)amino)-4-hydroxycyclopentane-1-carboxylate (150 g, 501 mmol). HNMR: (400 MHz CDCl₃) δ 4.81 (s, 1H), 4.28 (s, 1H), 3.67 (s, 3H), 3.08-3.15 (m, 1H), 2.07-2.27 (m, 1H), 2.02-2.05 (m, 2H), 1.84-1.86 (m, 2H), 1.44 (s, 9H).

### Step 7. Synthesis of Synthesis of (1S,3S,4R)-3-((tert-butoxycarbonyl)amino)-4-hydroxycyclopentane-1-carboxylic acid

To a solution of methyl (1S,3S,4R)-3-((tert-butoxycarbonyl)amino)-4-hydroxycyclopentane-1-carboxylate (170 g, 655 mmol) in MeOH (200 mL) and H₂O (200 mL) was added LiOH·H₂O (33.0 g, 786 mmol) at 20° C. and the suspension was stirred at 20° C. for 12 hours. After concentration in vacuo, the pH of the solution was adjusted to 4 with citric acid and it was extracted with ethyl acetate (100.0 mL*3). The combined organic layers were washed with brine (10.0 mL), dried over sodium sulfate, filtered and concentrated under reduced pressure. The crude product was triturated with petroleum ether:MTBE 3:1 (200 mL) at 20° C. for 30 minutes to give (1 S,3S,4R)-3-((tert-butoxycarbonyl)amino)-4-hydroxycyclopentane-1-carboxylic acid (93.5 g, 379 mmol). The mother liquor was purified by column chromatography (SiO₂, petroleum ether/ethyl acetate 5/1 to 0/1) to give more (1S,3S,4R)-3-((tert-butoxycarbonyl)amino)-4-hydroxycyclopentane-1-carboxylic acid (18.0 g, 72.9 mmol). ¹HNMR: (400 MHz CDCl₃) δ 5.11 (s, 1H), 4.25 (s, 1H), 4.00 (s, 1H), 3.11 (s, 1H), 2.24-2.32 (m, 1H), 1.91-2.09 (m, 2H), 1.82-1.88 (s, 1H), 1.43 (s, 9H).

### Example 7

### (3S,4R)-3-((tert-butoxycarbonyl)amino)-4-hydroxycyclopentane-1-carboxylic acid

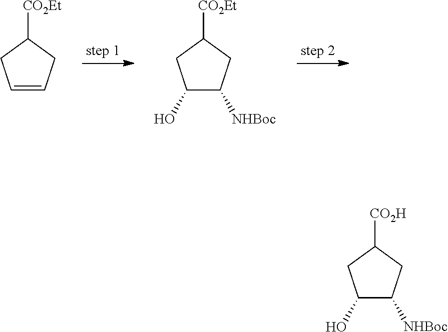 [SMILES: CCOC(=O)C1CC=CC1 ; CCOC(=O)C1C[C@@H](O)[C@@H](NC(=O)OC(C)(C)C)C1 ; CC(C)(C)OC(=O)N[C@H]1CC(C(=O)O)C[C@H]1O]

### Step 1. Synthesis of ethyl (3S,4R)-3-((tert-butoxycarbonyl)amino)-4-hydroxycyclopentane-1-carboxylate

To a mixture of BocNH₂ (98.4 g, 840 mmol) in n-propanol (690 mL) was added NaOH (0.5 M in water, 949 mL), tert-butyl hypochlorite (88.23 g, 813 mmol) at 25° C. The mixture was stirred at 25° C. for 30 minutes. A solution of ethyl cyclopent-3-ene-1-carboxylate (38.0 g, 271 mmol) and (DHQD)₂AQN (4.45 g, 5.42 mmol) in n-propanol (450 mL) was added, followed by K₂[OsO₂(OH)₄](2.00 g, 5.42 mmol) in aqueous NaOH solution (0.5 M, 152 mL) at 25° C. The mixture was stirred at 25° C. for 12 hours. The mixture was poured into H₂O (2.00 L) with stirring. The aqueous phase was extracted with ethyl acetate (1.00 L*3). The combined organic phase was dried over Na₂SO₄, filtered and concentrated under reduced pressure. The residue was purified by column chromatography (SiO₂, petroleum ether:ethyl acetate 30:1 to 0:1) and the fraction was concentrated under reduced pressure. The crude product was purified by preparative HPLC. The combined fractions were concentrated and the pH was adjusted to 7 with saturated aqueous NaHCO₃ solution. The solution was extracted with ethyl acetate (1.00 L*3). The combined organic layer was washed with brine (500 mL*2), dried over Na₂SO₄, filtered and concentrated under reduced pressure to give ethyl (3S,4R)-3-((tert-butoxycarbonyl)amino)-4-hydroxycyclopentane-1-carboxylate (42.0 g, 152 mmol) as a yellow oil. ¹H NMR (400 MHz, CDCl₃) δ 4.89 (d, J=6.4 Hz, 1H), 4.28 (s, 1H), 4.20-4.08 (m, 2H), 4.05-3.90 (m, 1H), 3.14-3.01 (m, 1H), 2.27-2.20 (m, 1H), 2.15-1.98 (m, 2H), 1.89-1.81 (m, 1H), 1.45 (s, 9H), 1.32-1.21 (m, 3H).

### Step 2. Synthesis of (3S,4R)-3-((tert-butoxycarbonyl)amino)-4-hydroxycyclopentane-1-carboxylic acid

This step was done similarly to step 7 in Example 6.

### Example 8

### (2s,4r)-6-oxo-5-azaspiro[3.4]octane-2-carboxylic acid and (2r,4s)-6-oxo-5-azaspiro[3.4]octane-2-carboxylic acid

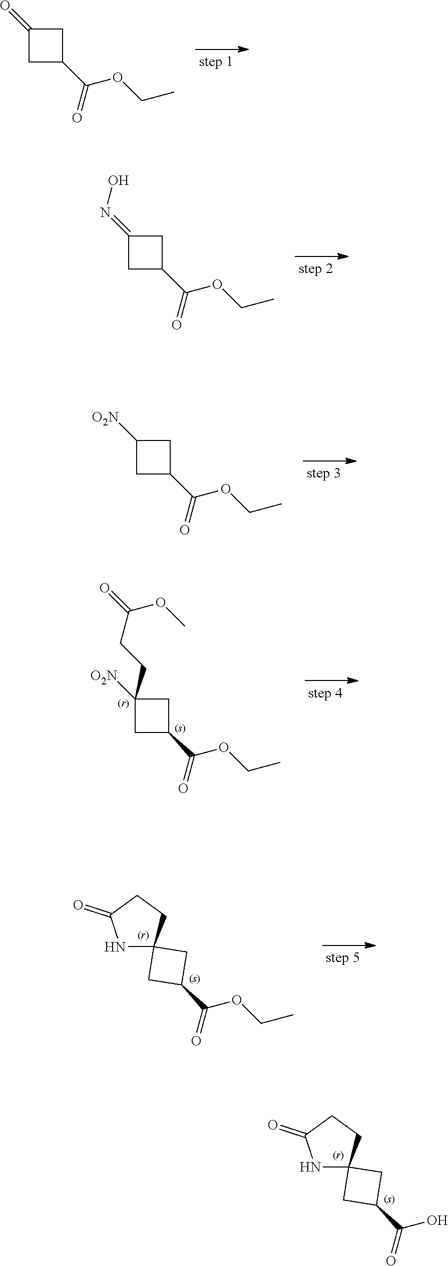

### Step 1. Synthesis of ethyl 3-(hydroxyimino)cyclobutane-1-carboxylate

To a solution of ethyl 3-oxocyclobutane-1-carboxylate (150 g, 1.06 mol) in EtOH (1.50 L) was added NH₂OH HCl (90.0 g, 1.30 mol) and NaOAc (106 g, 1.29 mol) at 20° C. The reaction mixture was heated to 90° C. and stirred at 90° C. for 15 hours. The reaction mixture was filtered and the filtrate was concentrated under reduced pressure. The residue was diluted with H₂O (1.00 L) and extracted with ethyl acetate (1.00 L*3). The combined organic layers were washed with brine (500 mL*3), dried over Na₂SO₄ and concentrated under reduced pressure. The residue was purified by column chromatography (silica gel, petroleum ether:ethyl acetate 30:1 to 1:1) to give ethyl 3-(hydroxyimino)cyclobutane-1-carboxylate (155 g, 986 mmol) as a colorless oil. ¹H NMR: (400 MHz, CDCl₃) δ 8.36 (br s, 1H), 4.18 (q, J=7.2 Hz, 2H), 3.21-3.16 (m, 5H), 1.28 (t, J=7.2 Hz, 3H).

### Step 2. Synthesis of ethyl 3-nitrocyclobutane-1-carboxylate

Two batches were carried out in parallel. To a mixture of ethyl 3-(hydroxyimino)cyclobutane-1-carboxylate (100 g, 636 mmol) in acetonitrile (600 mL) were Na₂HPO₄ (452 g, 3.18 mol) and urea-hydrogen peroxide (91.5 g, 973 mmol) added at 20° C., then to the mixture was added dropwise a solution of TFAA (266 mL, 1.91 mol) in acetonitrile (400 mL) at 30˜40° C. The mixture was heated to 80° C. and stirred at 80° C. for 1 hour. The two batches were worked up together. The reaction mixture was poured into H₂O (3.00 L) and extracted with ethyl acetate (1.50 L*2). The combined organic layers were washed by Na₂SO₃ solution (10%, 1.50 L*2), brine (1.00 L*2), concentrated under reduced pressure to give ethyl 3-nitrocyclobutane-1-carboxylate (130 g, 751 mmol) as a yellow oil. ¹H NMR: (400 MHz, CDCl₃) δ 5.11-4.82 (m, 1H), 4.22-4.16 (m, 2H), 3.44-3.25 (m, 1H), 2.97-2.80 (m, 4H), 1.32-1.25 (m, 3H).

### Step 3. Synthesis of ethyl (1s,3r)-3-(3-methoxy-3-oxopropyl)-3-nitrocyclobutane-1-carboxylate

To a mixture of ethyl 3-nitrocyclobutane-1-carboxylate (120 g, 693 mmol) in acetonitrile (1.20 L) was added methyl acrylate (246 mL, 2.73 mol) and DBU (104 mL, 693 mmol) dropwise at 0˜10° C. The mixture was warmed to 20° C. and stirred for 2 hours. The reaction mixture was quenched with aqueous NH₄Cl solution (10%, 3.00 L) and extracted with ethyl acetate (2.00 L*2). The combined organic layers were washed by brine (1.50 L*2) and concentrated under reduced pressure The residue was purified by preparative HPLC (column: Welch Ultimate XB-CN 250*50 mm, 10 μm; mobile phase A: hexane, mobile phase B: EtOH; gradient: 7% B isocratic) to give ethyl (1s,3r)-3-(3-methoxy-3-oxopropyl)-3-nitrocyclobutane-1-carboxylate

(38.0 g) as a yellow oil. ¹H NMR: (400 MHz, CDCl₃) δ 4.21-4.12 (m, 2H), 3.69 (s, 3H), 3.26-3.18 (m, 1H), 3.09-3.03 (m, 2H), 2.64-2.59 (m, 2H), 2.48-2.44 (m, 2H), 2.31-2.27 (m, 2H), 1.28 (t, J=7.2 Hz, 3H).

### Step 4. Synthesis of ethyl (2s,4r)-6-oxo-5-azaspiro[3.4]octane-2-carboxylate

To a mixture of compound ethyl (1s,3r)-3-(3-methoxy-3-oxopropyl)-3-nitrocyclobutane-1-carboxylate (38.0 g, 147 mmol) in EtOH (570 mL) was added acetic acid (83.8 mL, 1.47 mol) at 20° C., then iron powder (40.9 g, 733 mmol) was added to the mixture in portions at 50° C. The mixture was stirred at 50° C. for 12 hours. The mixture was cooled to 25° C. To the mixture was added H₂O (500 mL) and it was filtered. The filtrate was concentrated to remove EtOH. Then the mixture was extracted with ethyl acetate (500 mL*2). The combined organic layers were washed with brine (500 mL*2), aqueous NaHCO₃ solution (10%, 500 mL), brine (500 mL*2), concentrated under reduced pressure to give ethyl (2s,4r)-6-oxo-5-azaspiro[3.4]octane-2-carboxylate (25.0 g, 127 mmol) as a yellow solid. ¹H NMR: (400 MHz, DMSO-d₆) δ 8.15 (s, 1H), 4.10-4.02 (m, 2H), 3.04-2.99 (m, 1H), 2.41-2.32 (m, 4H), 2.11-2.09 (m, 2H), 2.04-1.91 (m, 2H), 1.20-1.16 (m, 3H).

### Step 5. Synthesis of (2s,4r)-6-oxo-5-azaspiro[3.4]octane-2-carboxylic acid

To a mixture of compound ethyl (2s,4r)-6-oxo-5-azaspiro[3.4]octane-2-carboxylate (25.0 g, 127 mmol) in MeOH (225 mL) was added a solution of NaOH (15.2 g, 380 mmol) in H₂O (75.0 mL). The mixture was stirred at 20° C. for 12 hours. The mixture's pH was adjusted to 4 with HCl (4 N). The solution was concentrated to remove MeOH, then the mixture was filtered and the filter cake was dried over vacuum (part 1). The filtrate was concentrated. To the residue was added MeOH (100 mL) and the suspension was filtered. The filtrate was concentrated and the residue was purified by prep-HPLC (column: Phenomenex Luna C18 (250*80 mm*15 μm); mobile phase A: water; mobile phase B: acetonitrile; gradient: 1%-20% B over 20 minutes) to give more product (part 2). Part 1 and part 2 were combined and to give (2s,4r)-6-oxo-5-azaspiro[3.4]octane-2-carboxylic acid (15.5 g, 91.4 mmol) as a yellow amorphous solid. ¹H NMR: (400 MHz, DMSO-d₆) δ 12.17 (s, 1H), 7.98 (s, 1H), 2.79-2.70 (m, 1H), 2.29-2.20 (m, 4H), 2.14-2.08 (m, 4H).

### Example 9

### (2r,4r)-6-oxo-7-oxa-5-azaspiro[3.4]octane-2-carboxylic acid

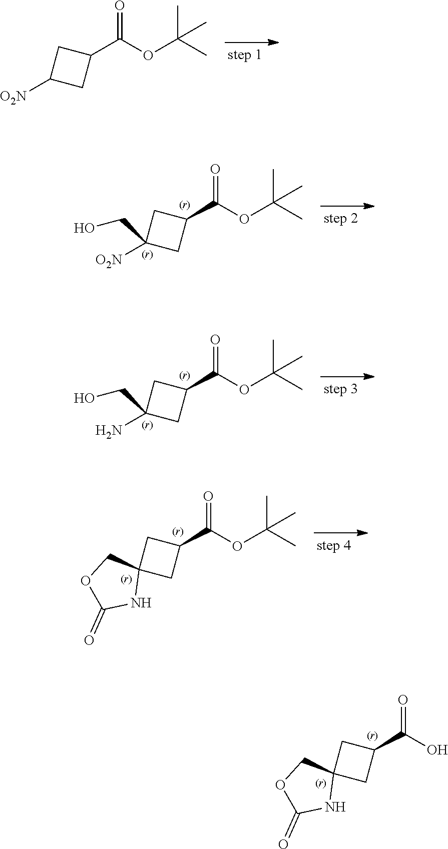 [SMILES: CC(C)(C)OC(=O)C1CC([N+](=O)[O-])C1 ; CC(C)(C)OC(=O)[C@H]1C[C@](CO)([N+](=O)[O-])C1 ; CC(C)(C)OC(=O)[C@H]1C[C@@](N)(CO)C1 ; CC(C)(C)OC(=O)[C@H]1C[C@@]2(COC(=O)N2)C1 ; O=C1N[C@]2(CO1)C[C@@H](C(=O)O)C2]

### Step 1. Synthesis of tert-butyl (1r,3r)-3-(hydroxymethyl)-3-nitrocyclobutane-1-carboxylate

To a solution of tert-butyl 3-nitrocyclobutane-1-carboxylate (81.0 g, 403 mmol) in acetonitrile (810 mL) was added (HCHO)n (48.6 g) at 25° C. To the mixture was added TEA (57.2 mL, 411 mmol) dropwise at 0° C. The mixture was stirred at 25° C. for 12 hours. The mixture was poured into water (2.00 L) and extracted with ethyl acetate (1.00 L*3). The combined organic layer was washed with brine (1.00 L), dried over Na₂SO₄, filtered and concentrated. The residue was purified by column chromatography (silica gel, petroleum ether/ethyl acetate 1/0 to 10/1) to give tert-butyl (1r,3r)-3-(hydroxymethyl)-3-nitrocyclobutane-1-carboxylate (22.4 g, 96.8 mmol) as a white solid. The other isomer is tert-butyl (1s,3s)-3-(hydroxymethyl)-3-nitrocyclobutane-1-carboxylate (49.2 g, 213 mmol). [0737]tert-butyl (1r,3r)-3-(hydroxymethyl)-3-nitrocyclobutane-1-carboxylate ¹H NMR: (400 MHz, CDCl₃) δ 4.10 (d, J=6.4 Hz, 2H), 3.27-3.16 (m, 1H), 3.03-2.93 (m, 2H), 2.68-2.58 (m, 2H), 2.26 (t, J=6.6 Hz, 1H), 1.47 (s, 9H).

tert-butyl (1s,3s)-3-(hydroxymethyl)-3-nitrocyclobutane-1-carboxylate ¹H NMR: (400 MHz, CDCl₃) δ 4.03 (d, J=6.4 Hz, 2H), 3.00-2.91 (m, 2H), 2.88-2.78 (m, 1H), 2.64-2.56 (m, 2H), 2.41 (t, J=6.6 Hz, 1H), 1.46 (s, 9H).

### Step 2. Synthesis of tert-butyl (1r,3r)-3-amino-3-(hydroxymethyl)cyclobutane-1-carboxylate

To a mixture of tert-butyl (1r,3r)-3-(hydroxymethyl)-3-nitrocyclobutane-1-carboxylate (38.5 g, 166 mmol) in isopropanol (400 mL) was added Raney Ni (8.00 g) under N₂ at 25° C. The mixture was degassed under vacuum and purged with H₂ 3 times. The mixture was heated to 70° C. and stirred at 70° C. under H₂ (50 psi) for 12 hours. The mixture was filtered and the filtrate was concentrated to give tert-butyl (1r,3r)-3-amino-3-(hydroxymethyl)cyclobutane-1-carboxylate (33.0 g, crude) as an off-white solid. ¹H NMR: (400 MHz, CDCl₃) δ 3.47 (s, 2H), 3.13-2.98 (m, 1H), 2.32-2.27 (m, 2H), 2.09-1.96 (m, 2H), 1.43 (s, 9H).

### Step 3. Synthesis of tert-butyl (2r,4r)-6-oxo-7-oxa-5-azaspiro[3.4]octane-2-carboxylate

To a solution of compound tert-butyl (1r,3r)-3-amino-3-(hydroxymethyl)cyclobutane-1-carboxylate (33.0 g, 164 mmol) in THF (330 mL) was added TEA (48.7 mL, 350 mmol) at 25° C. To the mixture was added a solution of triphosgene (17.3 g, 58.4 mmol) in THF (120 mL) dropwise at −10° C. and the mixture was stirred at −10° C. for 0.5 hour. The reaction mixture was warmed to 25° C. and stirred for 2 hours. The mixture was poured into cold water (1.50 L) and extracted with ethyl acetate (1.00 L*3). The combined organic layer was washed with brine (1.00 L), dried over Na₂SO₄, filtered and concentrated. The residue was purified by column chromatography (silica gel, petroleum ether/ethyl acetate 1/0 to 5/1) to give tert-butyl (2r,4r)-6-oxo-7-oxa-5-azaspiro[3.4]octane-2-carboxylate (27.6 g, 121 mmol) as a light yellow solid. ¹H NMR: (400 MHz, CDCl₃) δ 6.78 (s, 1H), 4.47 (s, 2H), 2.90-2.84 (m, 1H), 2.66-2.56 (m, 2H), 2.51-2.44 (m, 2H), 1.46 (s, 9H).

### Step 4. Synthesis of (2r,4r)-6-oxo-7-oxa-5-azaspiro[3.4]octane-2-carboxylic acid

To tert-butyl (2r,4r)-6-oxo-7-oxa-5-azaspiro[3.4]octane-2-carboxylate (25.6 g, 113 mmol) was added TFA (250 mL, 3.38 mol) at 0° C. The mixture was stirred at 25° C. for 6 hours. The reaction mixture was concentrated. The residue was triturated with petroleum ether/ethyl acetate 1/1 (100 mL) at 25° C. for 0.5 hour. The mixture was filtered and the filter cake was dried under vacuum. To the solid was added water (250 mL) and it was lyophilized to give (2r,4r)-6-oxo-7-oxa-5-azaspiro[3.4]octane-2-carboxylic acid (18.0 g, 99.6 mmol) an off-white amorphous solid. ¹H NMR: (400 MHz, DMSO-d6) (12.27 (br s, 1H), 8.21 (s, 1H), 4.28 (s, 2H), 2.94-2.80 (m, 1H), 2.48-2.36 (m, 4H).

### Example 10

### (2r,4S)—N—((S)-(3-chlorophenyl)(cyclopentyl)methyl)-5-oxo-6-azaspiro[3.4]octane-2-carboxamide and (2s,4R)—N—((S)-(3-chlorophenyl)(cyclopentyl)methyl)-5-oxo-6-azaspiro[3.4]octane-2-carboxamide

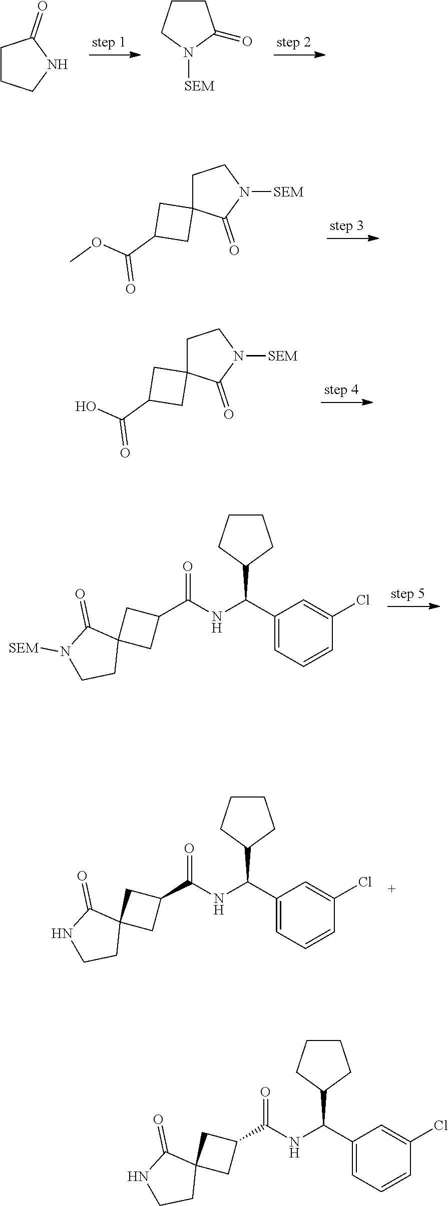

### Step 1. Synthesis of 1-((2-(trimethylsilyl)ethoxy)methyl)pyrrolidin-2-one

To a mixture of pyrrolidin-2-one (5 g, 0.059 mol) in THF (100 mL) was added NaH (1.69 g, 0.07 mol) in portions at 0° C. under a nitrogen atmosphere. The mixture was stirred for 1 h at 0° C. prior to the addition of (2-(chloromethoxy) ethyl) trimethylsilane (11.8 g, 0.07 mol) dropwise at 0° C. The mixture was stirred for 1 h at room temperature. The reaction was quenched with saturated NH₄Cl (aq.) and the aqueous phase was extracted with ethyl acetate (150 mL) three times. The combined organic layers were washed with brine, dried over sodium sulfate, filtered and concentrated in vacuo. The resulting crude material was purified by silica gel chromatography (100 g column; eluting with petroleum ether:ethyl acetate 5:1) to give 1-((2-(trimethylsilyl) ethoxy) methyl) pyrrolidin-2-one (4.0 g, 0.019 mol) as a yellow oil. ¹H NMR (400 MHz, DMSO-d6) δ 4.59 (s, 2H), 3.50-3.34 (m, 4H), 2.28 (t, J=8.0 Hz, 2H), 2.04-1.82 (m, 2H), 0.92-0.79 (m, 2H), 0.00 (s, 9H).

### Step 2. Synthesis of methyl 5-oxo-6-((2-(trimethylsilyl)ethoxy)methyl)-6-azaspiro[3.4]octane-2-carboxylate

To a mixture of 1-((2-(trimethylsilyl) ethoxy) methyl) pyrrolidin-2-one (5.0 g, 0.023 mol) in THF (100 mL) was added LDA (24.4 mL, 2 M in THF, 0.049 mol) dropwise at −78° C. under a nitrogen atmosphere. The mixture was stirred for 1 h at −78° C. prior to the addition of methyl 3-bromo-2-(bromomethyl) propanoate (6.0 g, 0.023 mol) dropwise at −78° C. The mixture was then stirred for 1 h at room temperature. The reaction was quenched with saturated NH₄Cl (aq.) and the aqueous phase was extracted with ethyl acetate (100 mL) three times. The combined organic layers were washed with brine, dried over sodium sulfate, filtered and concentrated in vacuo. The residue was purified by reverse phase flash chromatography (column: C18 silica gel; mobile phase A: water, B: Acetonitrile; gradient: 30% to 80% B in 10 min; detector: UV 220 nm to afford methyl 5-oxo-6-((2-(trimethylsilyl) ethoxy) methyl)-6-azaspiro [3.4]octane-2-carboxylate (200 mg, 0.64 mmol). LCMS RT 1.202 min, [M+H]⁺ 314.2, LCMS method C.

### Step 3. Synthesis of 5-oxo-6-((2-(trimethylsilyl)ethoxy)methyl)-6-azaspiro[3.4]octane-2-carboxylic acid

A mixture of methyl 5-oxo-6-((2-(trimethylsilyl) ethoxy) methyl)-6-azaspiro [3.4]octane-2-carboxylate (190 mg, 0.61 mmol) and NaOH (72.7 mg, 1.82 mmol) in MeOH/H₂O (1:1, 3 mL) was stirred for 1 h at room temperature. Concentration in vacuo gave the sodium salt of 5-oxo-6-((2-(trimethylsilyl)ethoxy)methyl)-6-azaspiro[3.4]octane-2-carboxylic acid (160 mg, 0.50 mmol) as a yellow oil. LCMS RT 0.655 min, [M+H]⁺ 300.2, LCMS method B.

### Step 4. Synthesis of (S)—N-((3-chlorophenyl)(cyclopentyl)methyl)-5-oxo-6-((2-(trimethylsilyl)ethoxy)methyl)-6-azaspiro[3.4]octane-2-carboxamide

A mixture of 5-oxo-6-((2-(trimethylsilyl) ethoxy) methyl)-6-azaspiro [3.4]octane-2-carboxylic acid (150 mg, 501 μmol), (S)-(3-chlorophenyl) (cyclopentyl)methanamine (105 mg, 501 μmol), DIEA (194 mg, 1.50 mmol) and HATU (381 mg, 1.00 mmol) in DMF (3 mL) was stirred for 1 h at room temperature. The reaction mixture was diluted with water (20 mL), and the aqueous phase was extracted with ethyl acetate (30 mL) three times. The combined organic layers were washed with brine, dried over sodium sulfate, filtered and concentrated in vacuo. The resulting crude material was purified by preparative HPLC (column: Xselect CSH C18 OBD Column 30*150 mm, 5 μm; mobile phase A: water (0.05% TFA), mobile phase B: acetonitrile; flow rate: 60 mL/min; gradient: 66% B to 75% B in 9 min, then 75% B; wavelength: 254/220 nm; RT: 7.15 min) to give (S)—N-((3-chlorophenyl) (cyclopentyl)methyl)-5-oxo-6-((2-(trimethylsilyl) ethoxy) methyl)-6-azaspiro [3.4]octane-2-carboxamide (70 mg, 0.14 mmol) as an off-white amorphous solid. LCMS RT 1.402 min, [M+H]⁺ 491.40, LCMS method B.

### Step 5. Synthesis of (2r,4S)—N—((S)-(3-chlorophenyl)(cyclopentyl)methyl)-5-oxo-6-azaspiro[3.4]octane-2-carboxamide and (2s,4R)—N—((S)-(3-chlorophenyl)(cyclopentyl)methyl)-5-oxo-6-azaspiro[3.4]octane-2-carboxamide

A mixture of (S)—N-((3-chlorophenyl) (cyclopentyl)methyl)-5-oxo-6-((2-(trimethylsilyl) ethoxy) methyl)-6-azaspiro [3.4]octane-2-carboxamide (50 mg, 0.10 mmol) in TFA (2 mL) was stirred for 1 h at room temperature. The mixture was concentrated in vacuo. Then the residue was dissolved in MeOH (1 mL). Ethylenediamine (61 mg, 1.0 mmol) was added, and the solution was stirred for 2 h at 80° C. After concentration in vacuo, the resulting crude material was purified by preparative HPLC (column: CHIRALPAK IC, 2*25 cm, 5 μm; mobile phase A: hexane, mobile phase B: EtOH; flow rate: 20 mL/min; gradient: 20% B; wavelength: 220/254 nm; RT1 (min): 9.15; RT2 (min): 14.23; sample solvent: EtOH; injection volume: 1.35 mL), then further purified by chiral preparative HPLC (column: DZ-CHIRALPAK ID-3, 4.6*50 mm, 3.0 μm; mobile phase: Hexane:EtOH 80:20; flow rate: 1 mL/min; gradient: isocratic; injection volume: 5 mL) to give one isomer (1 mg, 3 μmol) as an off-white amorphous solid. LCMS RT 1.496 min, [M+H]⁺ 361.2, LCMS method F; 1H NMR (400 MHz, DMSO) δ 1.08 (dq, J=17.0, 8.1 Hz, 1H), 1.27 (d, J=24.3 Hz, 3H), 1.37-1.67 (m, 5H), 1.80 (s, 1H), 1.95-2.07 (m, 2H), 2.31 (p, J=8.8 Hz, 2H), 2.50 (s, 1H), 2.52 (s, 1H), 2.74 (dd, J=14.4, 3.8 Hz, 1H), 3.04-3.17 (m, 2H), 4.58 (dd, J=10.5, 8.6 Hz, 1H), 5.36 (s, 1H), 5.59 (s, 1H), 7.24-7.38 (m, 3H), 7.45 (d, J=1.9 Hz, 1H), 7.61 (s, 1H), 8.49 (d, J=8.7 Hz, 1H). The other isomer (1 mg, 3 μmol) was also obtained as an off-white amorphous solid. LCMS RT 1.497 min, [M+H]⁺ 361.2, LCMS method F; 1H NMR (400 MHz, DMSO) δ 1.10 (dd, J=12.5, 8.3 Hz, 1H), 1.27 (dt, J=20.7, 6.5 Hz, 2H), 1.37-1.76 (m, 5H), 1.83 (s, 1H), 2.01 (td, J=15.5, 14.9, 10.7 Hz, 2H), 2.13-2.4 (m, 2H), 2.50 (s, 1H), 2.52 (s, 1H), 2.73 (dd, J=14.4, 3.8 Hz, 1H), 3.01-3.17 (m, 2H), 4.55 (dd, J=10.5, 8.6 Hz, 1H), 5.35 (s, 1H), 5.61 (s, 1H), 7.23-7.38 (m, 3H), 7.44 (d, J=1.7 Hz, 1H), 7.60 (s, 1H), 8.50 (d, J=8.6 Hz, IH).

### Example 11

### N—((R)-(3-chlorophenyl)(cyclopentyl)methyl)-7-fluoro-6-oxo-5-azaspiro[3.4]octane-2-carboxamide

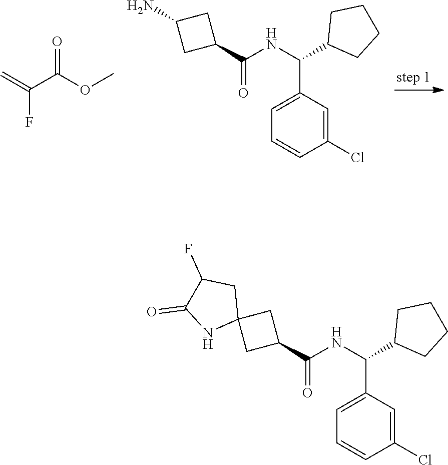

### Step 1. Synthesis of N—((R)-(3-chlorophenyl)(cyclopentyl)methyl)-7-fluoro-6-oxo-5-azaspiro[3.4]octane-2-carboxamide

To a 4-mL vial there was added (1r,3R)-3-amino-N—((R)-(3-chlorophenyl)(cyclopentyl)methyl)cyclobutane-1-carboxamide (50 mg, 0.16 mmol), tetrabutylammonium azide (4.6 mg, 16 μmol), 4CzIPN (1.3 mg, 1.6 μmol), and Cs₂CO₃ (53 mg, 0.16 mmol). The vial was capped and purged with nitrogen. Acetonitrile (1.1 mL) was added. The vial was sparged with nitrogen and while sparging, methyl 2-fluoroacrylate (15 μL, 0.16 mmol) was added via syringe. The reaction was then placed in the Merch photoreactor for 16 hours at 100% light intensity. The solution was concentrated and placed on the AccQ prep system eluting with 30-60% water with 0.1% formic acid to give N—((R)-(3-chlorophenyl)(cyclopentyl)methyl)-7-fluoro-6-oxo-5-azaspiro[3.4]octane-2-carboxamide (3.3 mg, 8.7 μmol) as an off-white solid. LCMS RT 1.44 min, [M+H]+379.23, LCMS method K.

### Example 12

### (2r,4R)—N—((R)-(3-chlorophenyl)(cyclopentyl)methyl)-8-(difluoromethyl)-6-oxo-5-azaspiro[3.4]octane-2-carboxamide and (2s,4S)—N—((R)-(3-chlorophenyl)(cyclopentyl)methyl)-8-(difluoromethyl)-6-oxo-5-azaspiro[3.4]octane-2-carboxamide

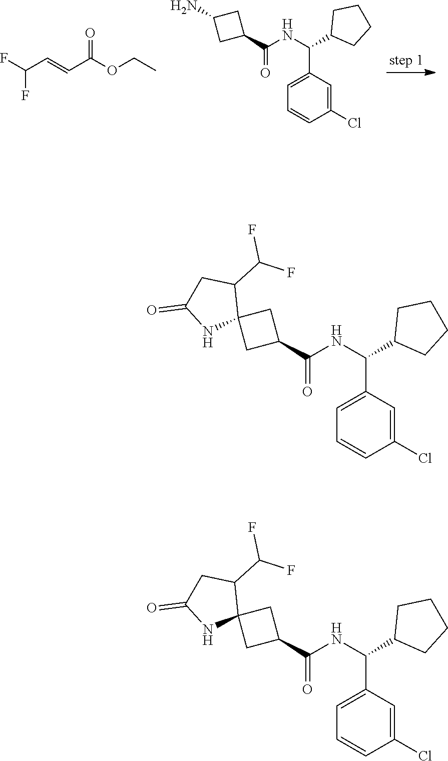

### Step 1. Synthesis of (2r,4R)—N—((R)-(3-chlorophenyl)(cyclopentyl)methyl)-8-(difluoromethyl)-6-oxo-5-azaspiro[3.4]octane-2-carboxamide and (2s,4S)—N—((R)-(3-chlorophenyl)(cyclopentyl)methyl)-8-(difluoromethyl)-6-oxo-5-azaspiro[3.4]octane-2-carboxamide

To a 8-mL vial there was added (1r,3R)-3-amino-N—((R)-(3-chlorophenyl)(cyclopentyl)methyl)cyclobutane-1-carboxamide (150 mg, 489 μmol) which was stirred with Cs₂CO₃ (159 mg, 489 μmol) in MeOH for 1 hour before filtering off the Cs₂CO₃ to convert the material to the free base. Tetrabutylammonium azide (13.9 mg, 48.9 μmol) and 4CzIPN (3.86 mg, 4.89 μmol) were added. The vial was capped and purged with nitrogen and dissolved in acetonitrile (2 mL). The vial was sparged with nitrogen and while sparging ethyl (E)-4,4-difluorobut-2-enoate (66.5 μL, 489 μmol) was added via syringe. The reaction was then placed in the Merch photoreactor for 8 hours at 100% light intensity. Reaction was concentrated and the vial was placed on the AccQ prep system, eluting with 20-50% water with 0.1% formic acid to give (2r,4R)—N—((R)-(3-chlorophenyl)(cyclopentyl)methyl)-8-(difluoromethyl)-6-oxo-5-azaspiro[3.4]octane-2-carboxamide and (2s,4S)—N—((R)-(3-chlorophenyl)(cyclopentyl)methyl)-8-(difluoromethyl)-6-oxo-5-azaspiro[3.4]octane-2-carboxamide, both as an off-white solid. Peak 1: 4.2 mg, LCMS RT 1.51 min, [M+H]⁺ 411.34, LCMS method K.

Peak 2: 5 mg, LCMS RT 1.53 min, [M+H]⁺ 411.34, LCMS method K.

### Example 13

### N-((1S,2R,4S)-4-(((S)-(3-chloro-2,6-difluorophenyl)(4-fluorobicyclo[2.2.1]heptan-1-yl)methyl)carbamoyl)-2-hydroxycyclopentyl)pyrimidine-5-carboxamide and N-((1S,2R,4R)-4-(((S)-(3-chloro-2,6-difluorophenyl)(4-fluorobicyclo[2.2.1]heptan-1-yl)methyl)carbamoyl)-2-hydroxycyclopentyl)pyrimidine-5-carboxamide

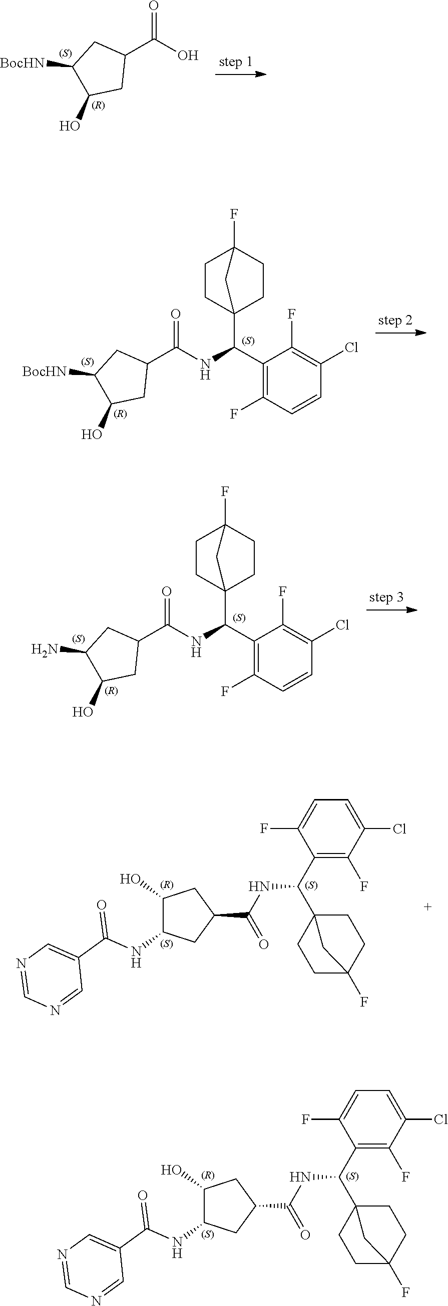

### Step 1. Synthesis of (3S,4R)-3-((tert-butoxycarbonyl)amino)-4-hydroxycyclopentane-1-carboxylic acid

A round bottomed flask was charged with (3S,4R)-3-((tert-butoxycarbonyl)amino)-4-hydroxycyclopentane-U-carboxylic acid (110 mg, 449 μmol), (S)-(3-chloro-2,6-difluorophenyl)(4-fluorobicyclo[2.2.1]heptan-1-yl)methanamine (130 mg, 449 μmol), T3P (256 mg, 673 μmol), TEA (113 mg, 1.35 mmol) and a stirbar. DMF (1 mL) was added, and the solution was stirred for 1 h at 25° C. The reaction mixture was diluted with water (50 mL), and the aqueous phase was extracted with ethyl acetate three times. The combined organic layers were washed with brine, dried over sodium sulfate, filtered and concentrated in vacuo. The residue was purified by reverse phase flash chromatography with the following condition: column: C18 silica gel; mobile phase A: water, mobile phase B: acetonitrile; gradient: 0% to 100% B in 10 min; detector: UV 220 nm. Lyophilization yielded tert-butyl ((1S,2R)-4-(((S)-(3-chloro-2,6-difluorophenyl)(4-fluorobicyclo[2.2.1]-heptan-1-yl)methyl)carbamoyl)-2-hydroxycyclopentyl)carbamate (150 mg, 290 gmol) as an amorphous off-white solid. LCMS RT 1.094 min, [M+H]⁺ 517, LCMS method D.

### Step 2. Synthesis of (3S,4R)-3-amino-N—((S)-(3-chloro-2,6-difluorophenyl)(4-fluorobicyclo[2.2.1]heptan-1-yl)methyl)-4-hydroxycyclopentane-1-carboxamide

A round bottomed flask was charged with tert-butyl ((1S,2R)-4-(((S)-(3-chloro-2,6-difluorophenyl)(4-fluorobicyclo[2.2.1]heptan-1-yl)methyl)carbamoyl)-2-hydroxycyclopentyl)carbamate (1.5 g, 2.9 mmol) and a stirbar. HCl (15 mL, 4 molar in MeOH, 60 mmol) was added, and the solution was stirred for 30 minutes at 25° C. Concentration in vacuo resulted in (3S,4R)-3-amino-N—((S)-(3-chloro-2,6-difluorophenyl)(4-fluorobicyclo[2.2.1]heptan-1-yl)methyl)-4-hydroxycyclopentane-1-carboxamide (1.0 g, 2 mmol, crude) as a white solid. No workup was performed. LCMS RT 0.898 min, [M+H]⁺ 417.25, LCMS method D.

### Step 3. Synthesis of N-((1S,2R,4S)-4-(((S)-(3-chloro-2,6-difluorophenyl)(4-fluorobicyclo [2.2.1]heptan-1-yl)methyl)carbamoyl)-2-hydroxycyclopentyl)pyrimidine-5-carboxamide and N-((1S,2R,4R)-4-(((S)-(3-chloro-2,6-difluorophenyl)(4-fluorobicyclo[2.2.1]heptan-1-yl)methyl)carbamoyl)-2-hydroxycyclopentyl)pyrimidine-5-carboxamide

A mixture of (3S,4R)-3-amino-N—((S)-(3-chloro-2,6-difluorophenyl)(4-fluorobicyclo [2.2.1]heptan-1-yl)methyl)-4-hydroxycyclopentane-1-carboxamide (350 mg, 840 μmol), pyrimidine-5-carboxylic acid (104 mg, 840 μmol), NaHCO₃ (212 mg, 2.52 mmol) and HATU (638 mg, 1.68 mmol) in DMF (5 mL) was stirred for 1 hour at 25° C. The reaction mixture w as diluted with water (10 mL), and the aqueous phase was extracted with ethyl acetate (10 m L) three times. The combined organic layers were washed with brine, dried over sodium sulfate, filtered and concentrated in vacuo. The residue was purified by reverse phase flash chromatography (column: C18 silica gel; mobile phase A: water; mobile phase B: acetonitrile. Gradient: 40% to 60% B in 10 min; detector: UV 220 nm, which afforded N-((1S,2R)-4-(((S)-(3-chloro-2,6-difluorophenyl)(4-fluorobicyclo[2.2.1]heptan-1-yl)methyl)carbamoyl)-2-hydroxycyclopentyl)pyrimidine-5-carboxamide (335 mg, 76.3%) as an off-white amorphous sol id. LCMS RT 0.842 min, [M+H]523, LCMS method C.

The resulting material was purified by chiral preparative HPLC (column: CHIRALPAKIF3; mobile phase A: hexane (0.2% DEA); B: MeOH:DCM 1:1) gradient: 75:25 isocratic; flow rate: 1 mL/min; injection volume: 3 mL). Lyophilization yielded one isomer (12 mg, 23 μmol) as an off-white amorphous solid and the other isomer (15 mg, 29 μmol, 60%), also as an off-white amorphous solid. Peak 1: ¹H NMR (400 MHz, DMSO-d₆) δ 9.29 (d, J=1.2 Hz, 1H), 9.17 (d, J=1.1 Hz, 2H), 8.43 (d, J=7.5 Hz, 1H), 8.26 (d, J=8.2 Hz, 1H), 7.57 (td, J=8.7, 5.4 Hz, 1H), 7.15 (t, J=9.1 Hz, 1H), 5.29 (d, J=8.1 Hz, 1H), 4.88 (d, J=3.4 Hz, 1H), 4.20-4.12 (m, 1H), 4.10 (d, J=3.9 Hz, 1H), 3.17 (dt, J=13.0, 6.4 Hz, 1H), 2.06-1.88 (m, 2H), 1.86-1.61 (d, J=8.4 Hz, 1OH), 1.47 (d, J=8.3 Hz, 2H). LCMS RT 1.398 min, [M+H]⁺ 523, LCMS method D. Peak 2: ¹H NMR (400 MHz, DMSO-d₆) δ 9.28 (s, 1H), 9.15 (s, 2H), 8.42 (d, J=7.2 Hz, 1H), 8.26 (d, J=8.3 Hz, 1H), 7.57 (td, J=8.6, 5.4 Hz, 1H), 7.15 (td, J=9.5, 1.6 Hz, 1H), 5.28 (d, J=8.1 Hz, 1H), 4.88 (d, J=3.2 Hz, 1H), 4.19-4.08 (m, 2H), 3.21-3.13 (m, 1H), 1.96-1.68 (m, 11H), 1.61 (d, J=8.5 Hz, 1H), 1.47 (d, J=8.9 Hz, 2H). LCMS RT 1.404 min, [M+H]⁺ 523, LCMS method D.

### Example 14

### (1S,3S,4R)-3-acetamido-N—((S)-(3-chloro-2,6-difluorophenyl)(4-fluorobicyclo[2.2.1]heptan-1-yl)methyl)-4-hydroxycyclopentane-1-carboxamide

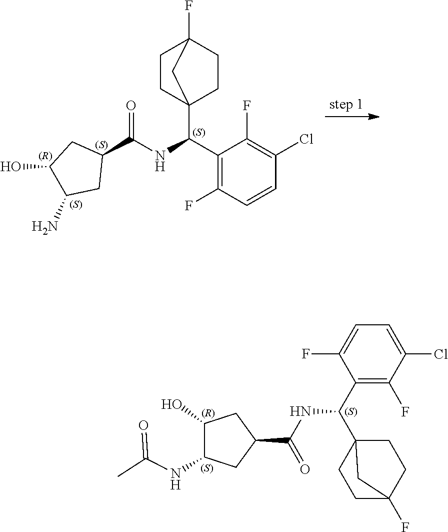

### Step 1. Synthesis of (1S,3S,4R)-3-acetamido-N—((S)-(3-chloro-2,6-difluorophenyl)(4-fluorobicyclo[2.2.1]heptan-1-yl)methyl)-4-hydroxycyclopentane-1-carboxamide

A round bottomed flask was charged with (1S,3S,4R)-3-amino-N—((S)-(3-chloro-2,6-difluorophenyl)(4-fluorobicyclo[2.2.1]heptan-1-yl)methyl)-4-hydroxycyclopentane-1-carboxamide (1.45 g, 3.48 mmol), acetic acid (209 mg, 3.48 mmol), NaHCO₃ (1.46 g, 17.4 mmol), HATU (2.64 g, 6.96 mmol) and a stir bar. DMF (15 mL) was added, and the solution was stirred for 1 hour at room temperature. The resulting crude material was purified by preparative HPLC (column: LuxCellulose-34.6*100 mm, 3 μm; mobile phase A: water, mobile phase B: MeOH (0.5% 2M NH₃ in MeOH); flow rate: 4 mL/min; gradient: 20% B isocratic) to give (1S,3S,4R)-3-acetamido-N—((S)-(3-chloro-2,6-difluorophenyl)(4-fluorobicyclo[2.2.1]heptan-1-yl)methyl)-4-hydroxycyclopentane-1-carboxamide (1.34 g, 2.93 mmol) as an off-white amorphous solid. ¹H NMR (400 MHz, DMSO-d₆) δ 8.22 (d, J=8.2 Hz, 1H), 7.61-7.48 (m, 2H), 7.19-7.09 (m, 1H), 5.26 (d, J=8.1 Hz, 1H), 4.78 (d, J=3.3 Hz, 1H), 3.96-3.85 (m, 2H), 3.15-3.03 (m, 1H), 1.82 (d, J=5.0 Hz, 2H), 1.81 (s, 3H), 1.75 (ddd, J=21.1, 11.5, 8.1 Hz, 8H), 1.59 (d, J=8.8 Hz, 2H), 1.45 (d, J=9.6 Hz, 2H). LCMS RT 0.833 min, [M+H]⁺ 459.05, LCMS method C.

### Example 15

### N-((1S,2R,4S)-4-(((S)-(2,3-dichloro-6-fluorophenyl)(4-fluorobicyclo[2.2.1]heptan-1-yl)methyl)carbamoyl)-2-hydroxycyclopentyl)-1,3,4-oxadiazole-2-carboxamide

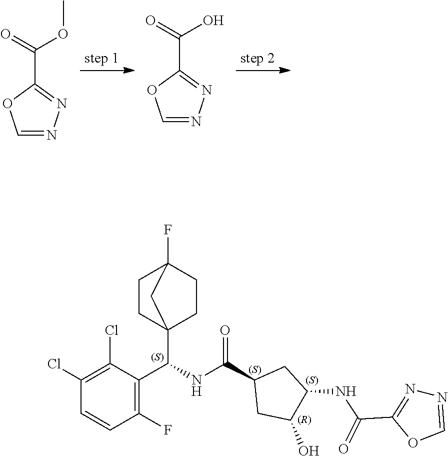

### Step 1. Synthesis of 1,3,4-oxadiazole-2-carboxylic acid

To a stirred mixture of methyl 1,3,4-oxadiazole-2-carboxylate (200 mg, 1.56 mmol) in THF (1 mL) and H₂O (1 mL) was added LiOH (74.8 mg, 3.12 mmol) at 25° C. under a nitrogen atmosphere. The resulting mixture was stirred for 1 hour at 25° C. under nitrogen. The mixture was acidified to pH 7 with HCl (aq. 1M). The resulting mixture was concentrated under reduced pressure to afford 1,3,4-oxadiazole-2-carboxylic acid (260 mg, 2.28 mmol, crude) as a white solid. LCMS RT 0.177 min, [M−H]⁻ 113.0, LCMS method E.

### Step 2. Synthesis of N-((1S,2R,4S)-4-(((S)-(2,3-dichloro-6-fluorophenyl)(4-fluorobicyclo[2.2.1]heptan-1-yl)methyl)carbamoyl)-2-hydroxycyclopentyl)-1,3,4-oxadiazole-2-carboxamide

To a stirred mixture of (1S,3S,4R)-3-amino-N—((S)-(2,3-dichloro-6-fluorophenyl)(4-fluorobicyclo[2.2.1]heptan-1-yl)methyl)-4-hydroxycyclopentane-1-carboxamide (150 mg, 346 μmol) and 1,3,4-oxadiazole-2-carboxylic acid (47.4 mg, 415 μmol) in DMF (2 mL) was added sodium bicarbonate (145 mg, 1.73 mmol) and HATU (395 mg, 1.04 mmol) at 25° C. under a nitrogen atmosphere. The resulting mixture was stirred for 2 hours at 25° C. The resulting mixture was filtered and purified by preparative HPLC (column: Xbridge Prep OBD C18 Column, 50*250 mm, 10 μm; mobile phase A: water (10 mM NH₄HCO₃+0.05% NH₄OH), mobile phase B: acetonitrile; flow rate: 100 mL/min; gradient: 25% B to 55% B in 8 min; wavelength: 254 nm/220 nm; RT (min): 9.58) to give a white solid. This was further purified by prep chiral HPLC (column: CHIRALPAK IG, 3*25 cm, 5 μm; mobile phase A: hexane:MTBE 1:1 (0.5% 2M NH₃ in MeOH), mobile phase B: MeOH; flow rate: 40 mL/min; gradient: 20% B isocratic; wavelength: 212/230 nm; RT1 (min): 4.62; RT2 (min): 6.96; sample solvent: MeOH; injection volume: 0.9 mL) to give N-((1S,2R,4S)-4-(((S)-(2,3-dichloro-6-fluorophenyl)(4-fluorobicyclo[2.2.1]heptan-1-yl)methyl)carbamoyl)-2-hydroxycyclopentyl)-1,3,4-oxadiazole-2-carboxamide (14.6 mg, 26.9 μmol) as a white solid. LCMS RT 1.838 min, [M−H]⁻ 527.10, LCMS method E. ¹H NMR (300 MHz, DMSO-d6) δ 9.44 (s, 1H), 8.50 (d, J=6.8 Hz, 1H), 8.26 (d, J=8.2 Hz, 1H), 7.63 (dd, J=9.0, 5.1 Hz, 1H), 7.28 (dd, J=10.6, 8.9 Hz, 1H), 5.52 (d, J=8.0 Hz, 1H), 5.12 (d, J=3.0 Hz, 1H), 4.11 (s, 2H), 3.16 (t, J=7.1 Hz, 1H), 2.03-1.46 (m, 14H). ¹⁹F NMR (282 MHz, DMSO-d6) δ−109.304, −173.540.

### Example 16

### (1S,3S,4R)-3-((1R,2S)-2-cyanocyclopropane-1-carboxamido)-N—((S)-(2,3-dichloro-6-fluorophenyl)((1R,3r,5S)-3-methylbicyclo[3.1.0]hexan-3-yl)methyl)-4-hydroxycyclopentane-1-carboxamide and (1S,3S,4R)-3-((1S,2R)-2-cyanocyclopropane-1-carboxamido)-N—((S)-(2,3-dichloro-6-fluorophenyl)((1R,3r,5S)-3-methylbicyclo[3.1.0]hexan-3-yl)methyl)-4-hydroxycyclopentane-1-carboxamide

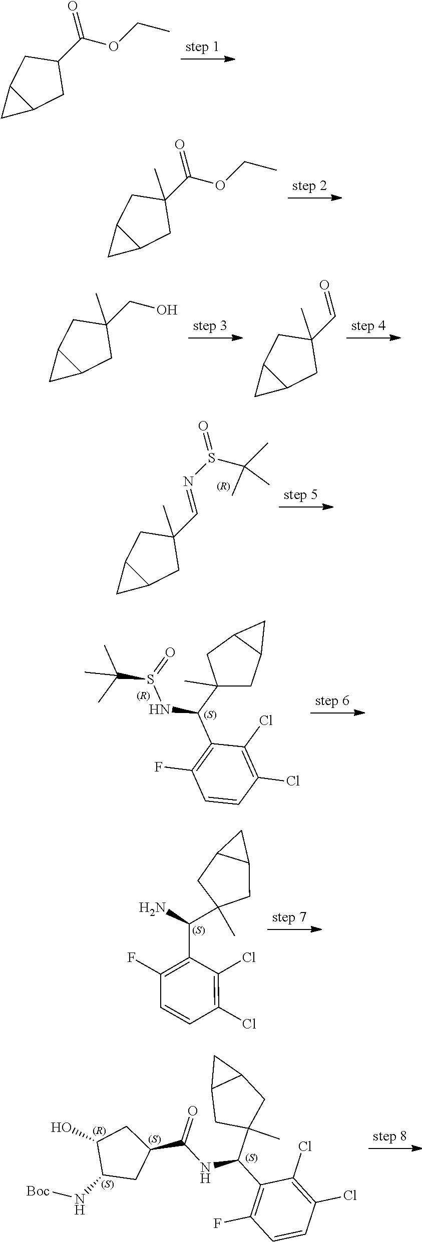

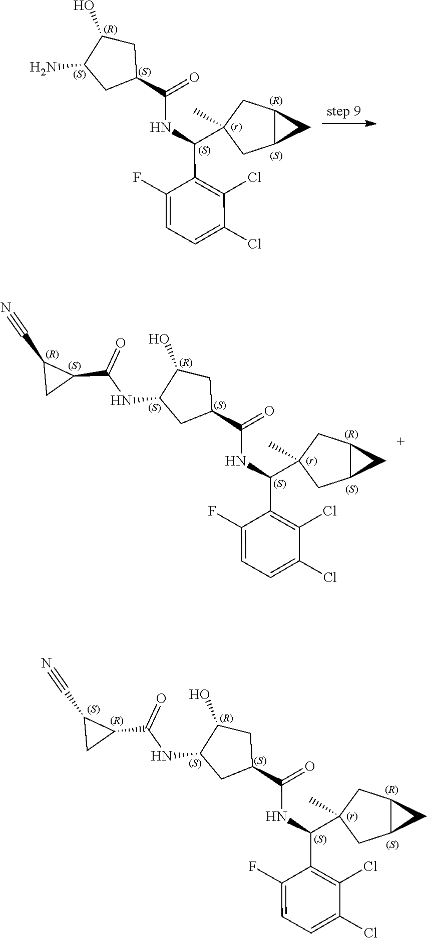

### Step 1. Synthesis of ethyl 3-methylbicyclo[3.1.0]hexane-3-carboxylate

To a mixture of ethyl bicycle [3.1.0]hexane-3-carboxylate (9.0 g, 0.058 mol) in THF (120 mL) was added LDA (45 ml, 2 M in THF, 0.09 mol) dropwise at −78° C. under a nitrogen atmosphere. The mixture was stirred for 1 h at −78° C. prior to addition of Mel (5 mL, 0.09 mol). The mixture was stirred for 2 h at 25° C. The reaction was quenched with saturated NH₄Cl (aq., 30 ml). The reaction mixture was diluted with water (100 mL), and the aqueous phase was extracted with ethyl acetate (100 mL*3). The combined organic layers were washed with brine, dried over sodium sulfate, filtered and concentrated in vacuo to give ethyl 3-methylbicyclo [3.1.0]hexane-3-carboxylate (9.0 g, 0.053 mol) as a yellow oil. GCMS RT 4.141 min, [M] 168.1, GC Method Z.

### Step 2. Synthesis of (3-methylbicyclo[3.1.0]hexan-3-yl)methanol

To a mixture of ethyl 3-methylbicyclo [3.1.0]hexane-3-carboxylate (10 g, 59 mmol) in THF (120 mL) was added LiAlH₄ (2.3 g, 59 mmol) in portions at 0° C. under a nitrogen atmosphere. The mixture was stirred for 2 hours at 25° C. The reaction was quenched with water (2.3 mL), NaOH (15%, 4.6 mL) and water (2.3 mL). The reaction mixture was filtered through a pad of Celite. The pad was washed with THF (100 mL), and the filtrate was concentrated in vacuo to give (3-methylbicyclo [3.1.0]hexan-3-yl) methanol (7.0 g) as a yellow oil. GCMS RT 3.760 min, [M] 126.0, GC Method Z.

### Step 3. Synthesis of 3-methylbicyclo[3.1.0]hexane-3-carbaldehyde

To a mixture of (3-methylbicyclo [3.1.0]hexan-3-yl) methanol (7.0 g, 55.47 mmol) in DCM (90 mL) was added PCC (13.15 g, 61.01 mmol) in portions at 0° C. under a nitrogen atmosphere. The mixture was stirred for 2 hours at 25° C. The reaction mixture was filtered (through pad of silica gel), the pad was washed with DCM. The filtrate was concentrated under reduced pressure to afford 3-methylbicyclo [3.1.0]hexane-3-carbaldehyde (6.0 g) as a brown oil. GCMS RT 3.484 min, [M] 124.1, GC Method Z.

### Step 4. Synthesis of (R)-2-methyl-N-((E)-(3-methylbicyclo[3.1.0]hexan-3-yl)methylene)propane-2-sulfinamide

To a solution of (R)-2-methylpropane-2-sulfinamide (6000 mg, 1 Eq, 49.50 mmol) and 3-methylbicyclo [3.1.0]hexane-3-carbaldehyde (6.762 g, 1.1 Eq, 54.46 mmol) in THF (75 mL) was added titanium(IV) isopropoxide (15.48 g, 16.5 mL, 1.1 Eq, 54.46 mmol). The mixture was heated at 50° C. for 16 hours. The reaction was quenched with water (100 mL). The reaction mixture was filtered (through a pad of Celite), the pad was washed with ethyl acetate (150 mL), and the filtrate was concentrated in vacuo.

The residue was purified by reverse phase flash chromatography (column: C18 silica gel; mobile phase A: water, mobile phase B: acetonitrile; 10% to 50% gradient in 10 min; detector: UV 220 nm. This resulted in (R)-2-methyl-N-((E)-(3-methylbicyclo [3.1.0]hexan-3-yl) methylene) propane-2-sulfinamide (9.2 g, 40 mmol, 82%) as a white solid. LCMS RT 0.997 min, [M+H]⁺ 228.15, LCMS method C.

### Step 5. Synthesis of (R)—N-((1S)-(2,3-dichloro-6-fluorophenyl)(3-methylbicyclo[3.1.0]hexan-3-yl)methyl)-2-methylpropane-2-sulfinamide

To a mixture of 1,2-dichloro-4-fluorobenzene (1.742 g, 10.56 mmol) in THF (25 mL) was added LDA (6.6 ml, 2M in THF, 13.2 mmol) dropwise at −78° C. under a nitrogen atmosphere. The mixture was stirred for 1 h at −78° C. prior to the addition of (R)-2-methyl-N-((E)-(3-methylbicyclo [3.1.0]hexan-3-yl) methylene) propane-2-sulfinamide (2.0 g, 8.796 mmol). The mixture was stirred for 2 h at 25° C. The reaction was quenched with saturated NH₄Cl (aq., 15 ml). The reaction mixture was diluted with water (20 mL), and the aqueous phase was extracted with ethyl acetate (50 mL*3). The combined organic layers were washed with brine, dried over sodium sulfate, filtered, and concentrated in vacuo. The residue was purified by reverse phase flash chromatography (column: C18 silica gel; mobile phase A: water, mobile phase B: acetonitrile; gradient: 10% to 50% B in 10 min; detector: UV 220 nm) to give (R)—N-((1S)-(2,3-dichloro-6-fluorophenyl) (3-methylbicyclo [3.1.0]hexan-3-yl) methyl)-2-methylpropane-2-sulfinamide (2.5 g, 6.4 mmol) as a yellow oil. LCMS RT 1.183 min, [M+H]⁺ 392, LCMS method A.

### Step 6. Synthesis of (1S)-(2,3-dichloro-6-fluorophenyl)(3-methylbicyclo[3.1.0]hexan-3-yl)methanamine

A mixture of (R)—N-((1S)-(2,3-dichloro-6-fluorophenyl) (3-methylbicyclo [3.1.0]hexan-3-yl) methyl)-2-methylpropane-2-sulfinamide (5.5 g, 14 mmol) and HCl (14 mL, 4 molar in MeOH, 56 mmol) was stirred for 1 h at 25° C. The mixture's pH was adjusted to 7-8 with saturated NaHCO₃ solution. The mixture was extracted with ethyl acetate (70 mL) three times. The combined organic layers were washed with brine, dried over sodium sulfate, filtered and concentrated in vacuo. This resulted in (1 S)-(2,3-dichloro-6-fluorophenyl) (3-methylbicyclo [3.1.0]hexan-3-yl) methanamine (3.8 g, 13 mmol) as a yellow oil. LCMS RT 0.738 min, [M+H]⁺ 288.0, LCMS method C.

### Step 7. Synthesis of tert-butyl ((1S,2R,4S)-4-(((1S)-(2,3-dichloro-6-fluorophenyl)(3-methylbicyclo[3.1.0]hexan-3-yl)methyl)carbamoyl)-2-hydroxycyclopentyl)carbamate

A mixture of (1S)-(2,3-dichloro-6-fluorophenyl) (3-methylbicyclo [3.1.0]hexan-3-yl) methanamine (4 g, 0.01 mol), (1S,3S,4R)-3-((tert-butoxycarbonyl) amino)-4-hydroxycyclopentane-1-carboxylic acid (3 g, 0.01 mol), HATU (8 g) and NaHCO₃ (3 g) in DMF (40 mL) was stirred for 1 h at 25° C. The reaction mixture was diluted with water (50 mL), and the aqueous phase was extracted with ethyl acetate (60 mL*3). The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 10% to 50% B in 10 min; detector: UV 220 nm) to give tert-butyl ((1S,2R,4S)-4-(((1S)-(2,3-dichloro-6-fluorophenyl) (3-methylbicyclo [3.1.0]hexan-3-yl) methyl) carbamoyl)-2-hydroxycyclopentyl) carbamate (5.1 g, 9.9 mmol) as an off-white solid. LCMS RT 1.080 min, [M+H]⁺ 515, LCMS method C.

### Step 8. Synthesis of (1S,3S,4R)-3-amino-N—((S)-(2,3-dichloro-6-fluorophenyl)((1R,3r,5S)-3-methylbicyclo[3.1.0]hexan-3-yl)methyl)-4-hydroxycyclopentane-1-carboxamide

A mixture of tert-butyl ((1 S,2R,4S)-4-(((1 S)-(2,3-dichloro-6-fluorophenyl) (3-methylbicyclo [3.1.0]hexan-3-yl) methyl) carbamoyl)-2-hydroxycyclopentyl) carbamate (2.0 g, 3.9 mmol) and HCl (19.40 mL, 4 N in MeOH, 77.60 mmol) in MeOH (20 mL) was stirred for 1 h at 25° C. The mixture's pH was adjusted to 7-8 with saturated NaHCO₃ solution. The reaction mixture was diluted with water (50 mL), and the aqueous phase was extracted with ethyl acetate (70 mL) three times. The combined organic layers were washed with brine, dried over sodium sulfate, filtered and concentrated in vacuo to give (1S,3S,4R)-3-amino-N-((1S)-(2,3-dichloro-6-fluorophenyl) (3-methylbicyclo [3.1.0]hexan-3-yl) methyl)-4-hydroxycyclopentane-1-carboxamide (1.5 g, 3.6 mmol) as a white amorphous solid. LCMS RT 0.780 min, [M+H]⁺ 415, LCMS method C.

### Step 9. Synthesis of (1S,3S,4R)-3-((1R,2S)-2-cyanocyclopropane-1-carboxamido)-N—((S)-(2,3-dichloro-6-fluorophenyl)((1R,3r,5S)-3-methylbicyclo[3.1.0]hexan-3-yl)methyl)-4-hydroxycyclopentane-1-carboxamide and (1S,3S,4R)-3-((1S,2R)-2-cyanocyclopropane-1-carboxamido)-N—((S)-(2,3-dichloro-6-fluorophenyl)((1R,3r,5S)-3-methylbicyclo[3.1.0]hexan-3-yl)methyl)-4-hydroxycyclopentane-1-carboxamide

A mixture of (1S,3S,4R)-3-amino-N—((S)-(2,3-dichloro-6-fluorophenyl)((1S,3r,5R)-3-methylbicyclo[3.1.0]hexan-3-yl)methyl)-4-hydroxycyclopentanecarboxamide (45 mg, 0.11 mmol), (±)-(1S,2R)-2-cyanocyclopropane-1-carboxylic acid (12 mg, 0.11 mmol), HATU (62 mg, 0.16 mmol) and NaHCO₃ (36 mg, 0.43 mmol) in DMF (1 mL) was stirred at room temperature for 1 hour. The reaction mixture was diluted with water (10 mL), and the aqueous phase was extracted with ethyl acetate (10 mL*3). The combined organic layers were washed with brine, dried over sodium sulfate, filtered and concentrated in vacuo. The resulting crude material was purified by preparative HPLC (column: XBridge Prep OBD C18 Column, 30*150 mm, 5 μm; mobile phase A: water (10 mM NH₄HCO₃)+0.05% NH₄OH, mobile phase B: acetonitrile; flow rate: 60 mL/min; gradient: 35% B to 62% B in 7 min; wavelength: 254 nm/220 nm; RT (min): 7.64) to give (1S,3S,4R)-3-((1S,2R)-2-cyanocyclopropane-1-carboxamido)-N—((S)-(2,3-dichloro-6-fluorophenyl) ((1R,3r,5S)-3-methylbicyclo [3.1.0]hexan-3-yl) methyl)-4-hydroxycyclopentane-1-carboxamide (51 mg, 0.10 mmol) as an off white amorphous solid. LCMS RT 1.118 min, [M+H]⁺ 508, LCMS method B. This material was further purified by chiral preparative HPLC (column: CHIRALPAK IE3; mobile phase A: hexane (0.2% diethylamine):(EtOH:DCM 1:1) 60:40; flow rate: 1 mL/min; gradient: isocratic; injection volume: 8 mL) to give (1S,3S,4R)-3-((1S,2R)-2-cyanocyclopropane-1-carboxamido)-N—((S)-(2,3-dichloro-6-fluorophenyl) ((1R,3r,5S)-3-methylbicyclo [3.1.0]hexan-3-yl) methyl)-4-hydroxycyclopentane-1-carboxamide and (1S,3S,4R)-3-((1R,2S)-2-cyanocyclopropane-1-carboxamido)-N—((S)-(2,3-dichloro-6-fluorophenyl) ((1R,3r,5S)-3-methylbicyclo [3.1.0]hexan-3-yl) methyl)-4-hydroxycyclopentane-1-carboxamide, both as an off white amorphous solid. One isomer is 10.5 mg (20.3 μmol), and the other is 12.5 mg (24.0 μmol). Isomer 1: ¹H NMR (400 MHz, DMSO-d₆) δ 8.12 (d, J=7.8 Hz, 1H), 8.01 (d, J=8.7 Hz, 1H), 7.60 (dd, J=9.0, 5.0 Hz, 11H), 7.25 (dd, J=10.7, 8.9 Hz, 1H), 5.41 (d, J=8.6 Hz, 1H), 4.88 (d, J=3.5 Hz, 1H), 3.98 (dt, J=14.8, 4.6 Hz, 2H), 3.13 (dt, J=14.0, 6.9 Hz, 1H), 2.23 (td, J=7.9, 6.3 Hz, 1H), 2.02 (td, J=8.6, 6.6 Hz, 1H), 1.86-1.59 (m, 5H), 1.52 (dd, J=12.8, 5.8 Hz, 1H), 1.44-1.21 (m, 6H), 1.14-1.02 (m, 3H), 0.85 (td, J=7.9, 4.0 Hz, 1H), 0.11 (q, J=3.9 Hz, 1H). LCMS RT 1.096 min, [M+H]⁺ 508, LCMS method B; isomer 2: ¹H NMR (400 MHz, DMSO-d₆) 8.07 (d, J=8.0 Hz, 1H), 7.98 (d, J=8.7 Hz, 1H), 7.60 (dd, J=9.0, 5.0 Hz, 1H), 7.24 (t, J=9.8 Hz, 1H), 5.41 (d, J=8.6 Hz, 1H), 4.93 (d, J=3.5 Hz, 1H), 4.10-3.91 (m, 2H), 3.14 (d, J=9.0 Hz, 1H), 2.26 (q, J=7.5 Hz, 1H), 2.03 (q, J=7.9 Hz, 1H), 1.80 (q, J=10.6, 7.9 Hz, 2H), 1.74-1.58 (m, 3H), 1.57-1.45 (m, 1H), 1.44-1.18 (m, 6H), 1.07 (s, 3H), 0.85 (s, 1H), 0.10 (d, J=4.1 Hz, 1H). LCMS RT 1.115 min, [M+H]⁺ 508, LCMS method B.

### Example 17

### (1S,3R)-3-acetamido-N—((S)-(2,3-dichloro-6-fluorophenyl)(1-methylcyclopentyl)methyl)-1-methylcyclopentane-1-carboxamide, (1R,3R)-3-acetamido-N—((S)-(2,3-dichloro-6-fluorophenyl)(1-methylcyclopentyl)methyl)-1-methylcyclopentane-1-carboxamide, (1S,3S)-3-acetamido-N—((S)-(2,3-dichloro-6-fluorophenyl)(1-methylcyclopentyl)methyl)-1-methylcyclopentane-1-carboxamide and (1R,3S)-3-acetamido-N—((S)-(2,3-dichloro-6-fluorophenyl)(1-methylcyclopentyl)methyl)-1-methylcyclopentane-1-carboxamide

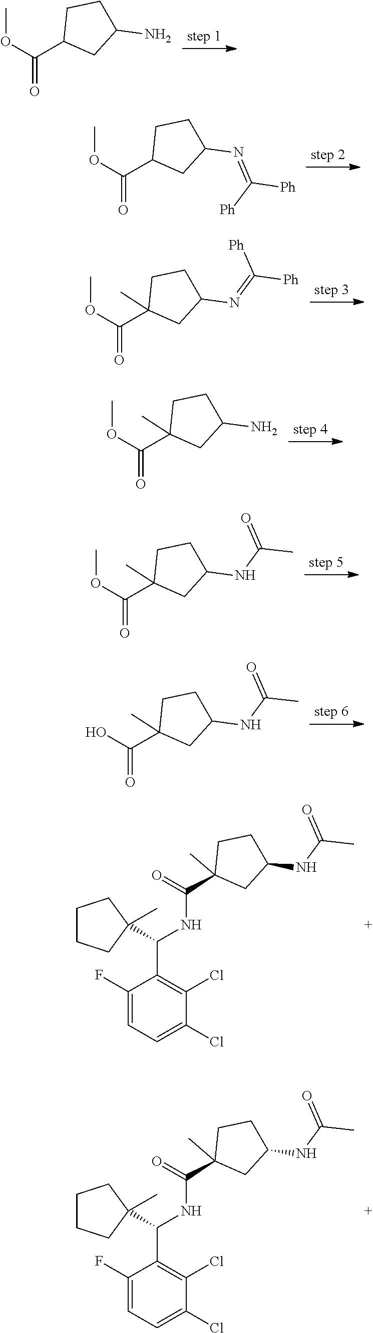

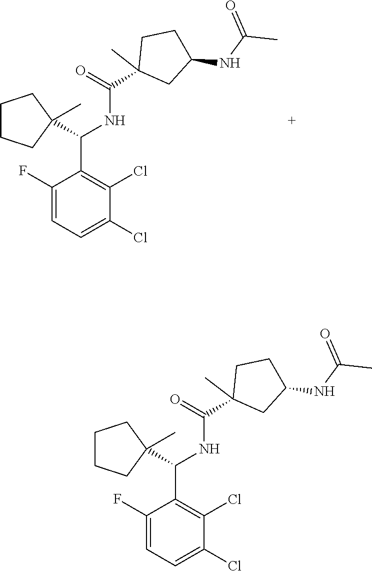 [SMILES: CC(=O)N[C@@H]1CC[C@@](C)(C(=O)N[C@H](c2c(F)ccc(Cl)c2Cl)C2(C)CCCC2)C1 ; CC(=O)N[C@H]1CC[C@@](C)(C(=O)N[C@H](c2c(F)ccc(Cl)c2Cl)C2(C)CCCC2)C1]

### Step 1. Synthesis of methyl 3-((diphenylmethylene)amino)cyclopentane-1-carboxylate

To a mixture of methyl 3-aminocyclopentane-1-carboxylate (4.6 g, 32 mmol) and T EA (18 mL, 0.13 mol) in DCM (50 mL) was added diphenylmethanimine (5.8 g, 32 mmol). The mixture was stirred at room temperature for 1 hour. The reaction mixture was filtered through a pad of Celite and the pad was washed with DCM (20 mL*3). The filtrate was concentrated in vacuo. The residue was purified by reverse phase flash chromatography (column: C18 silica gel; mobile phase A: water, mobile phase B: acetonitrile, gradient: 0% to 100% B in 20 min; detector: UV 254 nm) to give methyl 3-((diphenylmethylene) amino) cyclopenta ne-1-carboxylate (6.0 g) as a yellow oil. LCMS RT 0.670 min, [M+H]⁺ 308, LCMS method C.

### Step 2. Synthesis of methyl 3-((diphenylmethylene)amino)-1-methylcyclopentane-1-carboxylate

To a mixture of methyl 3-((diphenylmethylene)amino) cyclopentane-1-carboxylate (2.0 g, 6.51 mmol) in THF (30 mL) was added lithium diisopropylamide (3.9 mL, 2 molar, 7.8 mmol) dropwise at −78° C. under a nitrogen atmosphere. The mixture was stirred at −78° C. for 30 min prior to the addition of iodomethane (1.02 g, 7.16 mmol) dropwise at −78° C. The mixture was stirred at 25° C. for 1 hour. The reaction was quenched with saturated NH₄Cl (a q., 6 mL). The reaction mixture was diluted with water (40 mL), and the aqueous phase was extracted with ethyl acetate (50 mL*3). The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 0% to 100% in 20 min; detector: UV 254 nm) to give methy 13-((diphenylmethylene)amino)-1-methylcyclopentane-1-carboxylate (1.5 g, 4.7 mmol) as a yellow oil. LCMS RT 0.712 min, [M+H]⁺ 322, LCMS method C.

### Step 3. Synthesis of methyl 3-amino-1-methylcyclopentane-1-carboxylate

A mixture of methyl 3-((diphenylmethylene)amino)-1-methylcyclopentane-1-carboxylate (1.5 g, 4.7 mmol) in HCl (20 ml, 4 N) was stirred at 80° C. for 1 hour. The mixture was concentrated under reduced pressure to give methyl 3-amino-1-methylcyclopentane-1-carboxylate (0.7 g, 4 mmol) as a yellow oil which was used in the next step directly without purification. LCMS RT 0.479 min, [M+H]⁺ 158, LCMS method C.

### Step 4. Synthesis of methyl 3-acetamido-1-methylcyclopentane-1-carboxylate

To a mixture of methyl 3-amino-1-methylcyclopentane-1-carboxylate (700 mg, 4.45 mmol) and TEA (3.72 mL, 26.7 mmol) in DCM (10 mL) was added acetyl chloride (315 mg, 4.01 mmol) dropwise at 0° C. The mixture was stirred at room temperature for 1 hour. The reaction was quenched with MeOH (3 mL). The solution was concentrated under reduced pressure. The residue was purified by reverse phase flash chromatography (column: C18 silica gel; mobile phase A: water, mobile phase B: acetonitrile; gradient: 0% to 100% B in 15 min; detector: UV 254 nm) to give methyl 3-acetamido-1-methylcyclopentane-1-carboxylate (590 mg, 2.96 mmol) as a yellow oil. LCMS RT 0.612 min, [M+H]⁺ 200, LCMS method C.

### Step 5. Synthesis of 3-acetamido-1-methylcyclopentane-1-carboxylic acid

A mixture of methyl 3-acetamido-1-methylcyclopentane-1-carboxylate (590 mg, 2.96 mmol) and NaOH (5 mL, 4 N, aq.) in MeOH (5 mL) was stirred at room temperature for 1 hour. The solution was concentrated under reduced pressure. The mixture was acidified to pH of 4-6 with HCl (4 N). The solution was concentrated under reduced pressure. The residue was purified by reverse phase flash chromatography (column: C18 silica gel; mobile phase A: water, mobile phase B: acetonitrile; gradient: 0% to 50% B in 10 min; detector: UV 254 nm) to give 3-acetamido-1-methylcyclopentane-1-carboxylic acid (510 mg, 2.75 mmol) as a yellow oil. LCMS RT 0.496 min, [M+H]⁺ 185, LCMS method C.

### Step 6. Synthesis of (1S,3R)-3-acetamido-N—((S)-(2,3-dichloro-6-fluorophenyl)(1-methylcyclopentyl)methyl)-1-methylcyclopentane-1-carboxamide, (1R,3R)-3-acetamido-N—((S)-(2,3-dichloro-6-fluorophenyl)(1-methylcyclopentyl)methyl)-1-methylcyclopentane-1-carboxamide, (1S,3S)-3-acetamido-N—((S)-(2,3-dichloro-6-fluorophenyl)(1-methylcyclopentyl)methyl)-1-methylcyclopentane-1-carboxamide and (1R,3S)-3-acetamido-N—((S)-(2,3-dichloro-6-fluorophenyl)(1-methylcyclopentyl)methyl)-1-methylcyclopentane-1-carboxamide

A mixture of (S)-(2,3-dichloro-6-fluorophenyl)(1-methylcyclopentyl)methanamine (500 mg, 1.81 mmol), 3-acetamido-1-methylcyclopentane-1-carboxylic acid (671 mg, 3.62 mmol), HATU (1.38 g, 3.62 mmol) and NaHCO₃ (0.61 g, 7.24 mmol) in DMF (5 mL) was stirred at room temperature for 1 hour. The reaction mixture was diluted with water (6 mL), and the aqueous phase was extracted with ethyl acetate (10 mL*3). The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 0% to 100% B in 20 min; detector: UV 254 nm) to give 3-acetamido-N—((S)-(2,3-dichloro-6-fluorophenyl) (1-methylcyclopentyl) methyl)-1-methylcyclopentane-1-carboxamide (570 mg, 1.29 mmol) as a yellow oil. LCMS RT 1.146 min, [M+H]⁺ 443, LCMS method C.

The material was further purified by chiral preparative HPLC (column: (R, R)-WHELK-01-Kromasi, 5*25 cm, 5 μm; mobile phase A: hexane (0.5% 2M NH3-MeOH), mobile phase B: EtOH; flow rate: 20 mL/min; gradient: 35% B isocratic; wavelength: 220/254 nm; RT1 (min): 5.41; RT2 (min): 7.55; sample solvent: EtOH; injection volume: 0.4 mL) to give 3 peaks, then the peak that was still a mixture was purified by chiral preparative HPLC again (column: CHIRALPAK IH, 2*25 cm, 5 μm; mobile phase A: hexane (0.5% 2M NH3-MeOH), mobile phase B: EtOH; flow rate: 20 mL/min; gradient: 50% B isocratic; wavelength: 220/254 nm; RT1 (min): 3.65; RT2 (min): 37.12; sample solvent: EtOH; injection volume: 2.65 mL) to give 4 compounds in total, all as a white amorphous solid. Product 1:15 mg, 34 μmol. ¹H NMR (400 MHz, DMSO-d₆) δ 7.77 (d, J=7.2 Hz, 1H), 7.63 (dd, J=9.0, 5.1 Hz, 1H), 7.29 (dd, J=11.0, 8.9 Hz, 1H), 7.17 (d, J=8.8 Hz, 1H), 5.53 (d, J=8.6 Hz, 1H), 4.07 (h, J=7.4 Hz, 1H), 2.11-2.00 (m, 1H), 1.89 (dt, J=16.1, 7.1 Hz, 2H), 1.80 (dd, J=13.1, 8.3 Hz, 1H), 1.73 (s, 3H), 1.61 (s, 6H), 1.61-1.51 (m, 1H), 1.45-1.26 (m, 2H), 1.24 (s, 2H), 1.18 (s, 3H), 0.99 (d, J=2.9 Hz, 3H). LCMS RT 1.042 min, [M+H]⁺ 443, LC Method C. Product 2: 4.9 mg, 11 μmol. ¹H NMR (400 MHz, DMSO-d₆) δ 7.76 (d, J=7.4 Hz, 1H), 7.63 (dd, J=9.0, 5.1 Hz, 1H), 7.30 (dd, J=11.0, 9.0 Hz, 1H), 7.13 (d, J=9.0 Hz, 1H), 5.57 (d, J=8.8 Hz, 1H), 4.08 (h, J=7.6 Hz, 1H), 2.11 (ddd, J=12.6, 8.8, 6.0 Hz, 1H), 1.88 (ddd, J=24.6, 12.7, 6.9 Hz, 2H), 1.73 (s, 3H), 1.76-1.64 (m, 1H), 1.61 (s, 6H), 1.39 (ddt, J=36.1, 20.2, 7.5 Hz, 2H), 1.31 (s, 2H), 1.20 (s, 3H), 0.99 (d, J=2.9 Hz, 3H). LCMS RT 1.042 min, [M+H]⁺ 443, LCMS method C. Product 3: 80 mg, 0.18 mmol. ¹H NMR (400 MHz, DMSO-d₆) δ 7.82 (d, 0.1=7.3 Hz, 1H), 7.62 (dd, J=9.0, 5.1 Hz, 1H), 7.32-7.19 (m, 2H), 5.51 (d, J=8.7 Hz, 1H), 3.92 (h, J=7.7 Hz, 1H), 2.34 (dd, J=13.2, 8.1 Hz, 1H), 2.00 (dt, J=12.6, 7.6 Hz, 1H), 1.84-1.71 (m, 1H), 1.75 (s, 3H), 1.60 (s, 6H), 1.61-1.49 (m, 1H), 1.42 (dq, J=15.3, 7.4 Hz, 2H), 1.30 (s, 1H), 1.25 (s, 3H), 1.21 (dd, J=13.1, 7.6 Hz, 1H), 0.99 (d, J=2.9 Hz, 3H). LCMS RT 1.452 min, [M+H]⁺ 443, LCMS method B. Product 4: 64 mg, 0.14 mmol. ¹H NMR (400 MHz, DMSO-d₆) δ 7.82 (d, J=7.3 Hz, 1H), 7.62 (dd, J=8.9, 5.0 Hz, 1H), 7.37-7.19 (m, 2H), 5.50 (d, J=8.5 Hz, 1H), 3.92 (p, J=7.6, 7.0 Hz, 1H), 2.40 (dd, J=13.2, 8.0 Hz, 1H), 1.96 (dt, J=12.4, 7.4 Hz, 1H), 1.75 (s, 3H), 1.78-1.67 (m, 1H), 1.61 (s, 6H), 1.56-1.34 (m, 3H), 1.25 (s, 3H), 1.33-1.18 (m, 2H), 0.99 (d, J=2.9 Hz, 3H). LCMS RT 1.425 min, [M+H]⁺ 443, LCMS method B.

### Example 18

### (3aS,5S,6aR)—N—((S)-(3-chloro-2,6-difluorophenyl)(4-fluorobicyclo[2.2.1]heptan-1-yl)methyl)-2-oxohexahydro-2H-cyclopenta[d]oxazole-5-carboxamide and (3aS,5R,6aR)—N—((S)-(3-chloro-2,6-difluorophenyl)(4-fluorobicyclo[2.2.1]heptan-1-yl)methyl)-2-oxohexahydro-2H-cyclopenta[d]oxazole-5-carboxamide

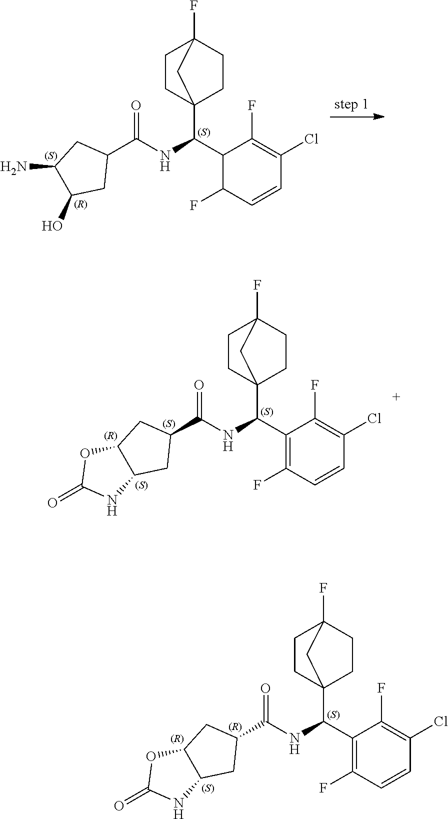

### Step 1. Synthesis of (3aS,5S,6aR)—N—((S)-(3-chloro-2,6-difluorophenyl)(4-fluorobicyclo[2.2.1]heptan-1-yl)methyl)-2-oxohexahydro-2H-cyclopenta[d]oxazole-5-carboxamide and (3aS,5R,6aR)—N—((S)-(3-chloro-2,6-difluorophenyl)(4-fluorobicyclo[2.2.1]heptan-1-yl)methyl)-2-oxohexahydro-2H-cyclopenta[d]oxazole-5-carboxamide

To a mixture of (3S,4R)-3-amino-N—((S)-(3-chloro-2,6-difluorophenyl)(4-fluorobicyclo[2.2.1]heptan-1-yl)methyl)-4-hydroxycyclopentane-1-carboxamide (50 mg, 0.12 mmol) and pyridine (47 mg, 0.60 mmol) in DCM (5 mL) was added a solution of triphosgene (18 mg, 60 μmol) in DCM (0.5 mL) dropwise at 0° C. The mixture was stirred for 12 hours at 25° C. The reaction was quenched with saturated NH₄Cl (aq.). The reaction mixture was diluted with water (10 mL), and the aqueous phase was extracted with ethyl acetate (10 mL*3). The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 10% to 80% B in 25 min; detector: UV 254 nm) to give (3aS,6aR)—N—((S)-(3-chloro-2,6-difluorophenyl)(4-fluorobicyclo[2.2.1]heptan-1-yl)methyl)-2-oxohexahydro-2H-cyclopenta[d]oxazole-5-carboxamide (35 mg, 79 μmol) as a white amorphous solid. LCMS RT 0.951 min, [M+H]⁻ 443.1, LCMS method C.

The material was purified by prep chiral-HPLC (column: CHIRALPAK-IG3; mobile phase A: hexane (0.2% diethylamine), mobile phase B: EtOH:DCM 1:1, gradient: 40% B isocratic; flow rate: 1 mL/min; injection volume: 3 mL) to give (3aS,5S,6aR)—N—((S)-(3-chloro-2,6-difluorophenyl)(4-fluorobicyclo[2.2.1]heptan-1-yl)methyl)-2-oxohexahydro-2H-cyclopenta[d]oxazole-5-carboxamide and (3aS,5R,6aR)—N—((S)-(3-chloro-2,6-difluorophenyl)(4-fluorobicyclo[2.2.1]heptan-1-yl)methyl)-2-oxohexahydro-2H-cyclopenta[d]oxazole-5-carboxamide, both as a white amorphous solid. One isomer is 5.3 mg, 12 μmol. ¹HNMR (400 MHz, CDCl₃) δ 7.49-7.26 (m, 1H), 6.88 (t, J=9.5 Hz, 1H), 6.74 (d, J=9.7 Hz, 1H), 5.64 (d, J=9.8 Hz, 1H), 5.45 (s, 1H), 5.26-4.93 (m, 1H), 4.42 (s, 1H), 3.00 (d, J=12.9 Hz, 1H), 2.38-2.16 (m, 1H), 2.10-1.31 (m, 13H). LCMS RT 0.882 min, [M+H]⁺ 443.1, LCMS method C; the other isomer is 11.5 mg, 26.0 μmol. ¹H NMR (400 MHz, DMSO-d6) 8.48 (d, J=8.5 Hz, 1H), 7.57 (td, J=8.6, 5.4 Hz, 2H), 7.32-7.00 (m, 1H), 5.27 (d, J=8.1 Hz, 1H), 5.14-4.81 (m, 1H), 4.19 (t, J=6.5 Hz, 1H), 3.11 (tt, J=12.0, 6.1 Hz, 1H), 2.09-1.87 (m, 1H), 1.88-1.31 (m, 13H). LCMS RT 0.879 min, [M+H]⁺ 443.1, LCMS method C.

### Example 19

### (1S,2R,4S)-4-acetamido-N—((S)-(2,3-dichloro-6-fluorophenyl)(1-methylcyclopentyl)methyl)-2-(hydroxymethyl)cyclopentane-1-carboxamide and (1R,2S,4R)-4-acetamido-N—((S)-(2,3-dichloro-6-fluorophenyl)(1-methylcyclopentyl)methyl)-2-(hydroxymethyl)cyclopentane-1-carboxamide

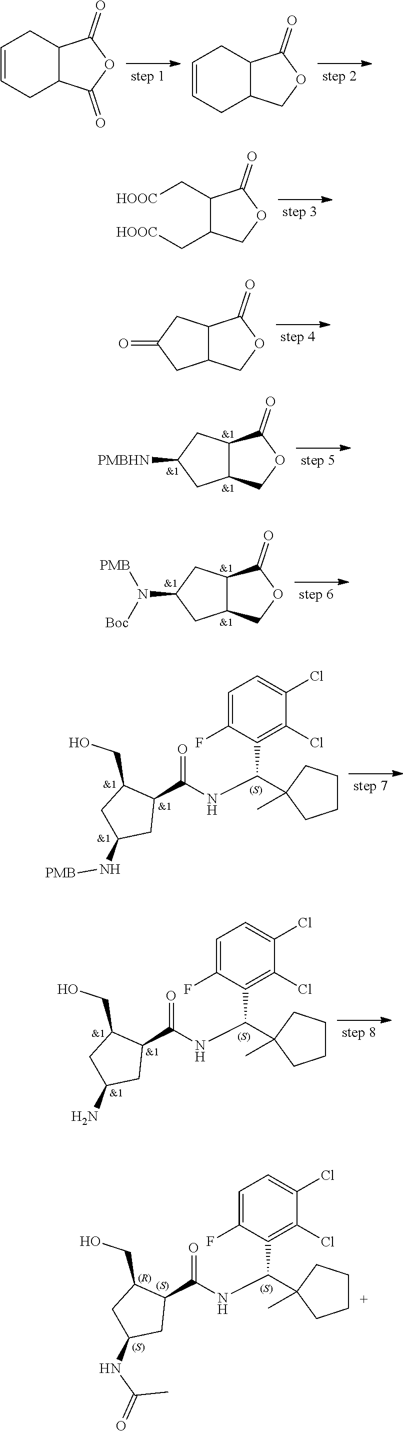

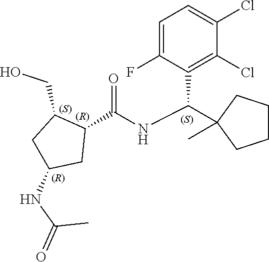

### Step 1. Synthesis of 3a,4,7,7a-tetrahydroisobenzofuran-1(3H)-one

To a mixture of 3a,4,7,7a-tetrahydroisobenzofuran-1,3-dione (20 g, 0.13 mol) in THF (200 mL) was added LiAlH₄ (5.0 g, 0.13 mol) in portions at 0° C. The mixture was stirred for 3 hours at room temperature. The resulting mixture was poured into 25 g of ice (mixed with 50 mL of 6% HCl in water) and extracted three times with ethyl acetate (200 ml*3). The combined organic layers were washed with brine and dried over anhydrous MgSO₄. The crude product was purified by silica gel chromatography (200 g column; eluting with petroleum ether/ethyl acetate; ratio: 10/1) to give 3a,4,7,7a-tetrahydroisobenzofuran-1(3H)-one (7 g, 0.05 mol) as a yellow oil. ¹H NMR (400 MHz, DMSO-d₆) δ 5.77-5.64 (m, 2H), 4.29 (dd, J=8.6, 4.9 Hz, 1H), 3.98 (dd, J=8.6, 1.5 Hz, 1H), 3.17 (d, J=5.2 Hz, 1H), 2.93 (td, J=7.3, 3.6 Hz, 1H), 2.64-2.52 (m, 1H), 2.44-2.01 (m, 3H).

### Step 2. Synthesis of 2,2′-(2-oxotetrahydrofuran-3,4-diyl)diacetic acid

To a mixture of KMnO₄ (15 g, 98 mmol) in H₂O (180 mL) was added a solution of 3a,4,7,7a-tetrahydroisobenzofuran-1(3H)-one (4.5 g, 33 mmol) in acetone (36 mL) dropwise at 0° C. The brown slurry was stirred for 1 h at 0° C., warmed to room temperature and stirred overnight. The reaction was quenched with NaHSO₃. The resulting slurry was filtered through a pad of Celite and the Celite was washed with water/THF (1/1, 250 mL). The combined filtrate was acidified to pH 2. The mixture was diluted with saturated NaCl (aq.) and extracted with tert-butyl methyl ether/THF (2/3, 6×120 mL). The combined organic layers were dried over Na₂SO₄, filtered and concentrated under reduced pressure (the bath temperature not exceeding 30° C.) to give 2,2′-(2-oxotetrahydrofuran-3,4-diyl)diacetic acid (5.5 g, 27 mmol) as an off-white solid. LCMS RT 0.238 min, [M+H]⁺ 203.05. LCMS method B.

### Step 3. Synthesis of tetrahydro-1H-cyclopenta[c]furan-1,5(3H)-dione

A mixture of 2,2′-(2-oxotetrahydrofuran-3,4-diyl) diacetic acid (7.3 g, 36 mmol) in acetic anhydride (50 mL) was stirred for 1 h at 130° C. After cooling to room temperature, the mixture was diluted with THF (10 mL) before K₂CO₃ (5.0 g, 36 mmol) was added. The resulting mixture was stirred at 60° C. overnight. After cooling to 0° C., the reaction was quenched with MeOH (5 mL) and the mixture was stirred for 30 min at 0° C. Saturated NH₄Cl solution (10 ml, aq.) and DCM (10 mL) were added and stirring continued for 20 min at 0° C. Phase separation followed by extraction of the aqueous layer with DCM (3×200 mL) gave a combined organic phase, which was dried over Na₂SO₄. The crude product was purified by silica gel chromatography (10 g column; eluting with petroleum ether/ethyl acetate; ratio: 1/1) to give tetrahydro-1H-cyclopenta[c]furan-1,5(3H)-dione (3.5 g, 25 mmol) as a pale yellow solid. ¹H NMR (400 MHz, Chloroform-d) δ 4.54 (dd, J=9.6, 5.9 Hz, 1H), 4.25 (dd, J=9.6, 1.9 Hz, 1H), 3.44-3.23 (m, 2H), 2.82-2.54 (m, 3H), 2.35-2.14 (m, 1H).

### Step 4. Synthesis of (f)-(3aS,5R,6aR)-5-((4-methoxybenzyl)amino)hexahydro-1H-cyclopenta[c]furan-1-one

To a mixture of tetrahydro-1H-cyclopenta[c]furan-1,5(3H)-dione (3.5 g, 25 mmol) a nd (4-methoxyphenyl) methanamine (4.1 g, 30 mmol) in MeOH (20 mL) was added NaBH₃ CN (2.4 g, 37 mmol) in portions at 0° C. The resulting mixture was stirred for 1 h at room temperature. The reaction mixture was diluted with water (120 mL), and the aqueous phase was extracted with ethyl acetate (150 mL) three times. The combined organic layers were washed with brine, dried over sodium sulfate, filtered and concentrated in vacuo. The resulting crude material was purified by flash chromatography (acetonitrile/water) to give (±)-(3aS,5R,6aR)-5-((4-methoxybenzyl)amino)hexahydro-1H-cyclopenta[c]furan-1-one (850 mg, 3.25 m mol) as colorless oil. LCMS RT 0.451 min, [M+H]⁺ 262, LCMS method C.

### Step 5. Synthesis of (±)-tert-butyl (4-methoxybenzyl)((3aS,5R,6aR)-1-oxohexahydro-1H-cyclopenta[c]furan-5-yl)carbamate

To a mixture of (3aS,5R,6aR)-5-((4-methoxybenzyl) amino) hexahydro-1H-cyclopenta[c]furan-1-one (630 mg, 2.41 mmol) and triethylamine (732 mg, 7.23 mmol) in DCM (10 mL) was added di-tert-butyl dicarbonate (789 mg, 3.62 mmol) dropwise at 0° C. The mixture was stirred for 2 hours at room temperature. The reaction mixture was diluted with water (20 mL), and the aqueous phase was extracted with ethyl acetate (30 mL) three times. The combined organic layers were washed with brine, dried over sodium sulfate, filtered and concentrated in vacuo. The resulting crude material was purified by flash chromatography (acetonitrile/water) to give (±)-tert-butyl (4-methoxybenzyl) ((3aS,5R,6aR)-1-oxohexahydro-1H-cyclopenta[c]furan-5-yl) carbamate (500 mg, 1.38 mmol, 57.4%) as an off-white solid. LCMS RT 1.178 min), [M+H]+=361, LCMS method C.

### Step 6. Synthesis of (±)-(1S,2R,4S)—N—((S)-(2,3-dichloro-6-fluorophenyl)(1-methylcyclopentyl)methyl)-2-(hydroxymethyl)-4-((4-methoxybenzyl)amino)cyclopentane-1-carboxamide

To a mixture of (±)-tert-butyl (4-methoxybenzyl)((3aS,5R,6aR)-1-oxohexahydro-1H-cyclopenta[c]furan-5-yl)carbamate (450 mg, 1.25 mmol) in THF (5 mL) was added trimethylaluminum (359 mg, 4.98 mmol) dropwise at 0° C. under a nitrogen atmosphere. The mixture was stirred for 15 min at 0° C. prior to the addition of (S)-(2,3-dichloro-6-fluorophenyl) (1-methylcyclopentyl) methanamine (1.38 g, 4.98 mmol). The mixture was stirred for 2 h at 50° C. The reaction mixture was diluted with water (10 mL), and the aqueous phase was extracted with ethyl acetate (15 mL) three times. The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 0% to 100% B in 10 min; detector: UV 220 nm) to give (+)-(1 S,2R,4S)—N—((S)-(2,3-dichloro-6-fluorophenyl)(1-methylcyclopentyl)methyl)-2-(hydroxymethyl)-4-((4-methoxybenzyl)amino)cyclopentane-1-carboxamide (150 mg, 279 μmol) as a yellow oil. LCMS RT 0.909 min, [M+H]⁺ 537.20, LCMS method C.

### Step 7. Synthesis of (±)-(1S,2R,4S)-4-amino-N—((S)-(2,3-dichloro-6-fluorophenyl)(1-methylcyclopentyl)methyl)-2-(hydroxymethyl)cyclopentane-1-carboxamide

A mixture of (±)-(1S,2R,4S)—N—((S)-(2,3-dichloro-6-fluorophenyl)(1-methylcyclopentyl)methyl)-2-(hydroxymethyl)-4-((4-methoxybenzyl)amino)cyclopentane-1-carboxamide (120 mg, 223 μmol) and Ce(NH₄)₂(NO₃)₆ (1.22 g, 2.23 mmol) in acetonitrile (10 mL) was stirred for 12 h at room temperature. The mixture was concentrated. The resulting crude material was purified by C18 flash (acetonitrile/water) to give (±)-(1S,2R,4S)-4-amino-N—((S)-(2,3-dichloro-6-fluorophenyl)(1-methylcyclopentyl)methyl)-2-(hydroxymethyl)cyclopentane-1-carboxamide (50 mg, 0.12 mmol) as a colorless oil. LCMS RT 0.750 min, [M+H]⁺ 417, LC MS method C.

### Step 8. Synthesis of (1S,2R,4S)-4-acetamido-N—((S)-(2,3-dichloro-6-fluorophenyl)(1-methylcyclopentyl)methyl)-2-(hydroxymethyl)cyclopentane-1-carboxamide and (1R,2S,4R)-4-acetamido-N—((S)-(2,3-dichloro-6-fluorophenyl)(1-methylcyclopentyl)methyl)-2-(hydroxymethyl)cyclopentane-1-carboxamide

A mixture of (±)-(1S,2R,4S)-4-amino-N—((S)-(2,3-dichloro-6-fluorophenyl)(1-methylcyclopentyl)methyl)-2-(hydroxymethyl)cyclopentane-1-carboxamide (45 mg, 0.11 mmol), TEA (45 μL, 0.32 mmol), acetic acid (13 mg, 0.22 mmol) and T₃P (51 mg, 0.16 mmol) in D MF (2 mL) was stirred for 1 h at room temperature. The reaction mixture was diluted with water (10 mL), and the aqueous phase was extracted with ethyl acetate (20 mL) three times. The combined organic layers were washed with brine, dried over sodium sulfate, filtered and concentrated in vacuo. The resulting crude material was purified by preparative HPLC (column: XBridge Shield RP18 OBD Column, 30*150 mm, 5 μm; mobile phase A: water (10 mM NH₄HCO₃+0.1% NH₄OH), mobile phase B: acetonitrile; flow rate: 60 mL/min; gradient 31% B to 51% B in 8 min, then 51% B; wavelength: 220/254 nm; RT1 (min): 7.40) to give (±)-(1R,2S,4R)-4-acetamido-N—((S)-(2,3-dichloro-6-fluorophenyl)(1-methylcyclopentyl)methyl)-2-(hydroxymethyl)cyclopentane-1-carboxamide (17 mg, 37 μmol) as a colorless oil. LCMS RT 1.077 min, [M+H]⁺ 459, LCMS method C. The material was further purified by chiral preparative HPLC (column: CHIRALPAK IC, 2*25 cm, 5 μm; mobile phase A: hexane (0.5% 2M NH₃ in MeOH), mobile phase B: EtOH:DCM 1:1; flow rate: 20 mL/min; gradient: 15% B isocratic; wavelength: 220/254 nm; RT1 (min): 15.95; RT2 (min): 21.01; sample solvent: EtOH:DCM 1:1; injection volume: 1 mL) to give (1 S,2R,4S)-4-acetamido-N—((S)-(2,3-dichloro-6-fluorophenyl)(1-methylcyclopentyl)methyl)-2-(hydroxymethyl)cyclopentane-1-carboxamide and (1R,2S,4R)-4-acetamido-N—((S)-(2,3-dichloro-6-fluorophenyl)(1-methylcyclopentyl)methyl)-2-(hydroxymethyl)cyclopentane-1-carboxamide, both as an off-white amorphous solid. Isomer 1 is 1 mg, 2 μmol. ¹HNMR (400 MHz, DMSO-d₆) δ 8.08 (d, J=8.7 Hz, 1H), 7.79 (d, J=7.1 Hz, 1H), 7.61 (dd, J=9.0, 4.9 Hz, 1H), 7.24 (t, J=9.9 Hz, 1H), 5.49 (d, J=8.5 Hz, 1H), 4.22 (t, J=5.1 Hz, 1H), 4.17 (s, 1H), 3.10 (d, J=7.4 Hz, 1H), 3.06-2.99 (m, 1H), 2.77 (q, J=6.7, 4.7 Hz, 1H), 2.29 (q, J=9.5, 8.7 Hz, 1H), 2.07 (dt, J=13.8, 6.7 Hz, 1H), 1.75 (s, 3H), 1.71 (t, J=7.0 Hz, 1H), 1.59 (s, 6H), 1.54-1.44 (m, 2H), 1.36 (s, 1H), 1.25 (s, 1H), 0.95 (s, 3H). LCMS RT 0.911 min, [M+H]459, LCMS method C. Isomer 22 is 2 mg, 4 μmol. ¹HNMR (400 MHz, DMSO-d₆) δ 8.11 (d, J=8.5 Hz, 1H), 7.80 (d, J=7.0 Hz, 1H), 7.61 (dd, J=9.0, 5.0 Hz, 1H), 7.25 (t, J=9.9 Hz, 1H), 5.46 (d, J=8.4 Hz, 1H), 4.50 (t, J=5.2 Hz, 1H), 4.14 (s, 1H), 3.44-3.36 (m, 1H), 3.21 (td, J=9.6, 5.6 Hz, 1H), 3.03 (q, J=8.0, 7.5 Hz, 1H), 2.42-2.32 (m, 1H), 1.93 (dd, J=13.1, 6.9 Hz, 1H), 1.74 (s, 3H), 1.61 (s, 6H), 1.53 (d, J=8.9 Hz, 2H), 1.40 (t, J=6.8 Hz, 2H), 1.24 (s, 1H), 1.00-0.91 (m, 3H). LCMS RT 0.933 min, [M+H]⁺ 459, LCMS method C.

### Example 20

### (1S,3R,4S)—N—((S)-(3-chloro-2,6-difluorophenyl)(4-fluorobicyclo[2.2.1]heptan-1-yl)methyl)-3-hydroxy-4-isopropoxycyclopentane-1-carboxamide, (1R,3S,4R)—N—((S)-(3-chloro-2,6-difluorophenyl)(4-fluorobicyclo[2.2.1]heptan-1-yl)methyl)-3-hydroxy-4-isopropoxycyclopentane-1-carboxamide, (1R,3R,4S)—N—((S)-(3-chloro-2,6-difluorophenyl)(4-fluorobicyclo[2.2.1]heptan-1-yl)methyl)-3-hydroxy-4-isopropoxycyclopentane-1-carboxamide and (1S,3S,4R)—N—((S)-(3-chloro-2,6-difluorophenyl)(4-fluorobicyclo[2.2.1]heptan-1-yl)methyl)-3-hydroxy-4-isopropoxycyclopentane-1-carboxamide

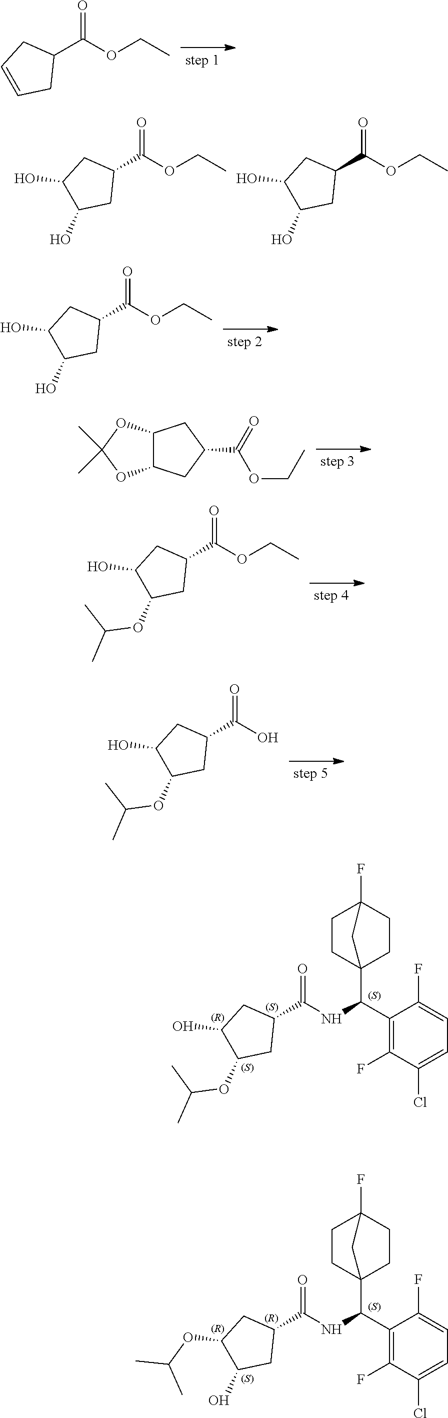

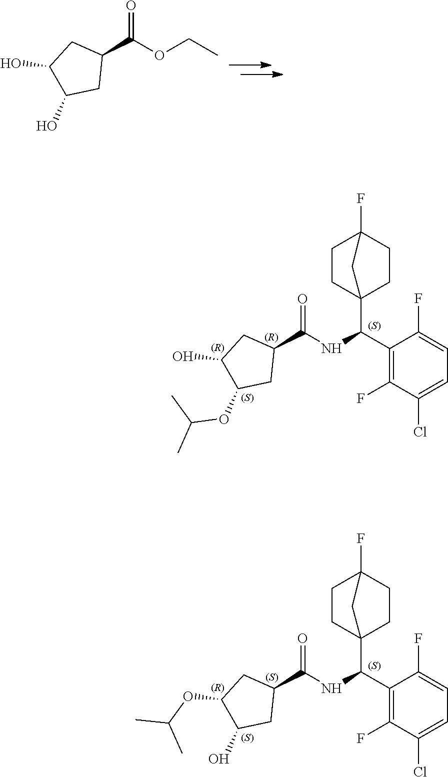

### Step 1. Synthesis of ethyl (1r,3R,4S)-3,4-dihydroxycyclopentane-1-carboxylate and ethyl (1s,3R,4S)-3,4-dihydroxycyclopentane-1-carboxylate

To a stirred mixture of ethyl cyclopent-3-ene-1-carboxylate (5 g, 0.04 mol) and NMO (5 g, 0.04 mol) in acetone (10 mL) and H₂O (10 mL) was added K₂OSO₂(OH)₄ (3 g, 7 mmol) at room temperature under a nitrogen atmosphere. The resulting mixture was stirred overnight at room temperature. The mixture was extracted with DCM (3×250 mL). The combined organic layers were washed with brine (1×100 mL), dried over anhydrous Na₂SO₄. After filtration, the filtrate was concentrated under reduced pressure. The residue was purified by silica gel column chromatography, eluting with petroleum ether:ethyl acetate (1:5) to afford ethyl (3R,4S)-3,4-dihydroxycyclopentane-1-carboxylate (4.14 g, 23.8 mmol, including 1.5 g isomer 1, 420 mg isomer 2, and 2.2 g mixture of the two) as a yellow oil. Isomer 1: ¹H NMR (400 MHz, DMSO-d6) δ 4.47 (d, J=4.2 Hz, 2H), 4.04 (q, J=7.1 Hz, 2H), 3.88 (h, J=4.0 Hz, 2H), 2.95 (tt, J=9.6, 6.7 Hz, 1H), 1.90-1.70 (m, 4H), 1.17 (t, J=7.1 Hz, 3H). Isomer 2: ¹H NMR (400 MHz, DMSO-d6) δ 4.37 (d, J=4.3 Hz, 2H), 4.04 (dd, J=7.1, 3.2 Hz, 2H), 3.76 (dp, J=7.5, 4.5 Hz, 2H), 2.67 (tt, J=9.3, 8.0 Hz, 1H), 1.95 (tdd, J=9.4, 4.8, 1.7 Hz, 2H), 1.83-1.76 (m, 2H), 1.17 (t, J=7.1 Hz, 3H).

### Step 2. Synthesis of ethyl (3aR,5r,6aS)-2,2-dimethyltetrahydro-4H-cyclopenta[d][1,3]dioxole-5-carboxylate

To a stirred mixture of ethyl (1r,3R,4S)-3,4-dihydroxycyclopentane-1-carboxylate (500 mg, 2.87 mmol, isomer 2) and 2,2-dimethoxypropane (299 mg, 2.87 mmol) in acetone (1 mL) was added 4-methylbenzene-1-sulfonic acid (98.9 mg, 574 μmol) at 25° C. under a nitrogen atmosphere. The resulting mixture was stirred for 16 hours at 25° C. under nitrogen. The resulting mixture was extracted with EtOAc (3×20 mL). The combined organic layers were washed with water (1×20 mL) and brine (1×20 mL), and dried over anhydrous Na₂SO₄. After filtration, the filtrate was concentrated under reduced pressure to afford ethyl (3aR,5r,6aS)-2,2-dimethyltetrahydro-4H-cyclopenta[d][1,3]dioxole-5-carboxylate (649 mg, 3.03 mmol, crude) as a colorless oil. 11H NMR (400 MHz, DMSO-d6) δ 4.62 (dd, J=3.7, 1.6 Hz, 2H), 4.06 (q, J=7.1 Hz, 2H), 2.89-2.80 (m, 1H), 1.98-1.87 (m, 2H), 1.67-1.59 (m, 2H), 1.34 (s, 3H), 1.23-1.12 (m, 6H).

### Step 3. Synthesis of (f)-ethyl (1S,3R,4S)-3-hydroxy-4-isopropoxycyclopentane-1-carboxylate

To a stirred mixture of ethyl (3aR,5r,6aS)-2,2-dimethyltetrahydro-4H-cyclopenta[d][1,3]dioxole-5-carboxylate (200 mg, 0.93 mmol) and triethylsilane (139 mg, 1.20 mmol) in DCM (5 mL) was added TiCl₄ (1.02 mL, 1 M in DCM, 1.02 mmol) dropwise at −40° C. under a nitrogen atmosphere. The resulting mixture was stirred at −40° C. for 1 hour under nitrogen. The reaction was quenched with water/ice at 0° C. The resulting mixture was extracted with DCM (3×50 mL). The combined organic layers were washed with brine (1×100 mL) and NaHCO₃ (1×100 mL), and dried over anhydrous Na₂SO₄. After filtration, the filtrate was concentrated under reduced pressure. The residue was purified by silica gel column chromatography, eluting with petroleum ether:ethyl acetate (5:1) to afford (±)-ethyl (1S,3R,4S)-3-hydroxy-4-isopropoxycyclopentane-1-carboxylate (120 mg, 555 μmol) as a colorless oil. ¹H NMR (400 MHz, DMSO-d6) δ 4.16 (d, J=4.4 Hz, 1H), 4.05 (q, J=7.1 Hz, 2H), 3.98 (q, J=4.3 Hz, 1H), 3.77 (td, J=6.9, 3.7 Hz, 1H), 3.70-3.64 (m, 1H), 3.00-2.87 (m, 1H), 1.94-1.76 (m, 4H), 1.17 (t, J=7.1 Hz, 3H), 1.09 (t, J=6.3 Hz, 6H).

### Step 4. Synthesis of (±)-(1S,3R,4S)-3-hydroxy-4-isopropoxycyclopentane-1-carboxylic acid

To a stirred mixture of (±)-ethyl (1S,3R,4S)-3-hydroxy-4-isopropoxycyclopentane-1-carboxylate (120 mg, 555 μmol) in MeOH (2 mL) and H₂O (2 mL) was added NaOH (44.4 mg, 1.11 mmol) at 25° C. under a nitrogen atmosphere. The resulting mixture was stirred for 1 hour at 25° C. under nitrogen. The mixture was acidified to pH 4 with conc. HCl. The resulting mixture was extracted with DCM (3×250 mL). The combined organic layers were washed with brine (1×100 mL) and dried over anhydrous Na₂SO₄. After filtration, the filtrate was concentrated under reduced pressure to afford (±)-(1S,3R,4S)-3-hydroxy-4-isopropoxycyclopentane-1-carboxylic acid (110 mg, 584 μmol). 1H NMR (300 MHz, DMSO-d6) δ 12.05 (s, 1H), 4.13 (d, J=4.4 Hz, 1H), 3.97 (p, J=4.3 Hz, 1H), 3.76 (td, J=7.0, 3.7 Hz, 1H), 3.70-3.62 (m, 1H), 2.87 (qd, J=8.6, 5.5 Hz, 1H), 1.95-1.74 (m, 4H), 1.09 (dd, J=6.1, 4.7 Hz, 6H).

### Step 5. Synthesis of (1S,3R,4S)—N—((S)-(3-chloro-2,6-difluorophenyl)(4-fluorobicyclo[2.2.1]heptan-1-yl)methyl)-3-hydroxy-4-isopropoxycyclopentane-1-carboxamide and (1R,3S,4R)—N—((S)-(3-chloro-2,6-difluorophenyl)(4-fluorobicyclo[2.2.1]heptan-1-yl)methyl)-3-hydroxy-4-isopropoxycyclopentane-1-carboxamide

To a stirred mixture of (±)-(1S,3R,4S)-3-hydroxy-4-isopropoxycyclopentane-1-carboxylic acid (100 mg, 531 μmol) and (S)-(3-chloro-2,6-difluorophenyl)(4-fluorobicyclo[2.2.1]heptan-1-yl)methanamine (169 mg, 584 μmol) in DMF (5 mL) was added T3P (507 mg, 50% wt. in EtOAc, 797 μmol) and TEA (69.9 mg, 691 μmol) at 25° C. under a nitrogen atmosphere. The resulting mixture was stirred for 1 hour at 25° C. under nitrogen. The resulting mixture was purified by preparative HPLC with the following conditions (column: XBridge Prep OBD C18 Column, 30*150 mm, 10 μm; mobile phase A: water (10 mM NH₄HCO₃+0.05% NH₄OH), mobile phase B: acetonitrile; flow rate: 60 mL/min; gradient: 35% B to 65% B in 30 min; wavelength: 254 nm/220 nm; RT (min): 9.58) to afford the desired product (140 mg, 304 μmol) as a white solid, which was further purified by preparative chiral HPLC (column: CHIRAL ART Cellulose-SZ, 3*25 cm, 5 μm; mobile phase A: hexane (0.5% of 2M NH₃ in MeOH), mobile phase B: EtOH; flow rate: 40 mL/min; gradient: 10% B isocratic; wavelength: 254/220 nm; RT1 (min): 8.63; RT2 (min): 10.525; sample solvent: EtOH:DCM 1:1) to give (1S,3R,4S)—N—((S)-(3-chloro-2,6-difluorophenyl)(4-fluorobicyclo[2.2.1]heptan-1-yl)methyl)-3-hydroxy-4-isopropoxycyclopentane-1-carboxamide and (1R,3S,4R)—N—((S)-(3-chloro-2,6-difluorophenyl)(4-fluorobicyclo[2.2.1]heptan-1-yl)methyl)-3-hydroxy-4-isopropoxycyclopentane-1-carboxamide, both as a white solid. Isomer 1: 34.6 mg, 73.4 μmol, LCMS RT 1.700 min, [M+H]⁺ 460.20, LCMS method F, ¹H NMR (400 MHz, DMSO-d6) δ 8.20 (d, J=8.4 Hz, 1H), 7.58 (td, J=8.7, 5.4 Hz, 1H), 7.16 (td, J=9.4, 1.6 Hz, 1H), 5.27 (d, J=8.3 Hz, 1H), 4.44 (d, J=5.1 Hz, 1H), 3.89 (p, J=4.7 Hz, 1H), 3.72-3.62 (m, 2H), 2.66 (qd, J=8.9, 6.2 Hz, 1H), 2.05-1.39 (m, 14H), 1.09 (dd, J=12.0, 6.0 Hz, 6H). Isomer 2: 46.0 mg, 97.5 μmol, LCMS RT 1.696 min, [M+H]⁺ 460.15, LCMS method F. ¹H NMR (300 MHz, DMSO-d6) δ 8.20 (d, J=8.3 Hz, 1H), 7.58 (td, J=8.7, 5.5 Hz, 1H), 7.16 (td, J=9.5, 1.7 Hz, 1H), 5.26 (d, J=8.2 Hz, 1H), 4.45 (d, J=5.2 Hz, 1H), 3.90 (p, J=4.6 Hz, 1H), 3.73-3.59 (m, 2H), 2.68 (qd, J=8.9, 6.1 Hz, 1H), 2.06-1.54 (m, 12H), 1.46 (s, 2H), 1.08 (dd, J=13.2, 6.1 Hz, 6H).

Similarly, (1R,3R,4S)—N—((S)-(3-chloro-2,6-difluorophenyl)(4-fluorobicyclo[2.2.1]heptan-1-yl)methyl)-3-hydroxy-4-isopropoxycyclopentane-1-carboxamide and (1S,3S,4R)—N—((S)-(3-chloro-2,6-difluorophenyl)(4-fluorobicyclo[2.2.1]heptan-1-yl)methyl)-3-hydroxy-4-isopropoxycyclopentane-1-carboxamide, both as a white solid, can be prepared from ethyl (1s,3R,4S)-3,4-dihydroxycyclopentane-1-carboxylate after chiral separation by preparative chiral HPLC with the following conditions (column: Lux Cellulose-4, 2.12*25 cm, 5 μm; mobile phase A: hexane (0.5% of 2 M NH₃ in MeOH), mobile phase B: EtOH; flow rate: 20 mL/min; gradient: 3% B isocratic; wavelength: 210/220 nm; RT1 (min): 5.49; RT2 (min): 7.80; sample solvent: EtOH; injection volume: 0.4 mL). Isomer 3: 22.2 mg, 47.4 μmol. LCMS RT 1.534 min, [M+H]⁺ 460.20, LCMS method F, ¹H NMR (400 MHz, DMSO-d6) δ 8.22 (d, J=8.2 Hz, 1H), 7.56 (td, J=8.7, 5.5 Hz, 1H), 7.15 (td, J=9.5, 1.6 Hz, 1H), 5.24 (d, J=8.1 Hz, 1H), 4.07 (d, J=4.3 Hz, 1H), 3.95 (p, J=4.2 Hz, 1H), 3.72-3.58 (m, 2H), 3.06 (ddd, J=15.8, 8.9, 6.2 Hz, 1H), 1.84-1.54 (m, 12H), 1.45 (d, J=9.2 Hz, 2H), 1.06 (dd, J=8.9, 6.1 Hz, 6H). Isomer 4: 34.2 mg, 72.0 μmol, LCMS RT 1.662 min, [M+H]⁺ 460.20, LCMS method F, ¹H NMR (400 MHz, DMSO-d6) δ 8.21 (d, J=8.1 Hz, 1H), 7.56 (td, J=8.7, 5.4 Hz, 1H), 7.19-7.08 (m, 1H), 5.24 (d, J=8.1 Hz, 1H), 4.06 (d, J=4.1 Hz, 1H), 3.92 (p, J=4.1 Hz, 1H), 3.73 (td, J=6.8, 3.6 Hz, 1H), 3.64 (h, J=6.1 Hz, 1H), 3.14-3.00 (m, 1H), 1.87-1.66 (m, 1OH), 1.59 (d, J=8.5 Hz, 1H), 1.48 (ddd, J=20.7, 10.3, 6.7 Hz, 3H), 1.08 (t, J=5.7 Hz, 6H).

### Example 21

### Synthesis of N-((1S,2R,4S)-4-(((S)-(3-chloro-2,6-difluorophenyl)(4-fluorobicyclo[2.2.1]heptan-1-yl)methyl)carbamoyl)-2-(methoxy-d3)cyclopentyl)pyrimidine-5-carboxamide and N-((1S,2R,4R)-4-(((S)-(3-chloro-2,6-difluorophenyl)(4-fluorobicyclo[2.2.1]heptan-1-yl)methyl)carbamoyl)-2-(methoxy-d3)cyclopentyl)pyrimidine-5-carboxamide

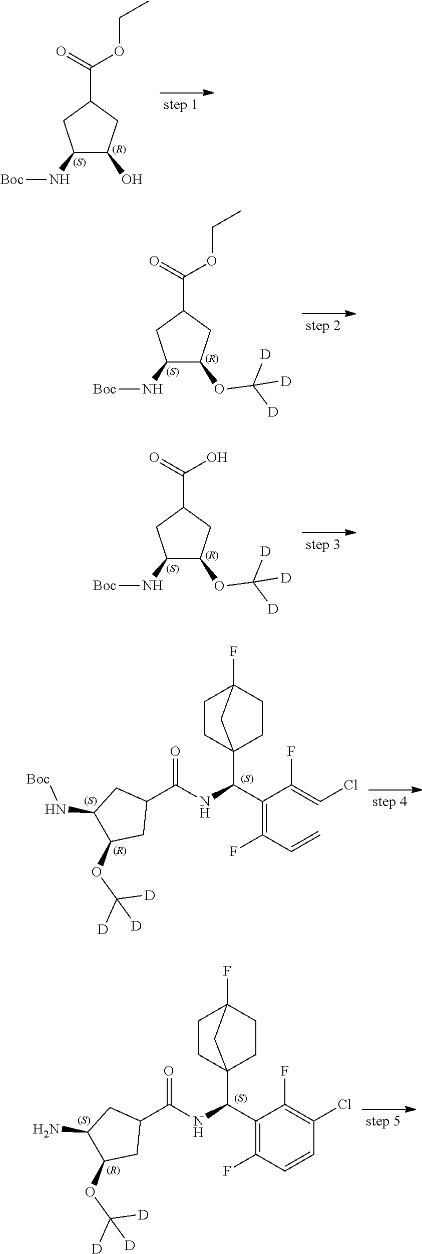

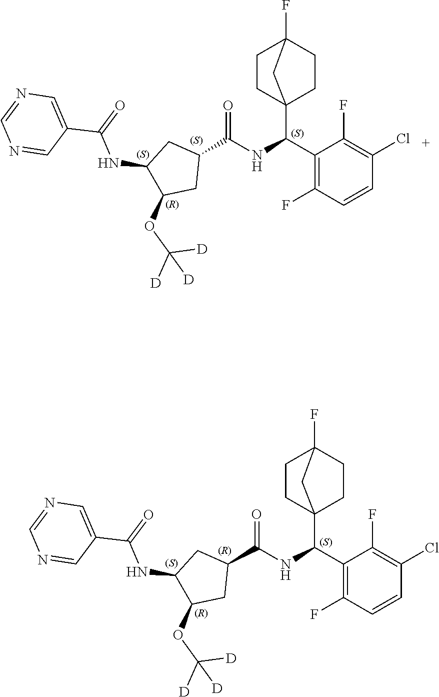

### Step 1. Synthesis of ethyl (3S,4R)-3-((tert-butoxycarbonyl)amino)-4-(methoxy-d3)cyclopentane-1-carboxylate

To a stirred solution of ethyl (3S,4R)-3-((tert-butoxycarbonyl)amino)-4-hydroxycyclopentane-1-carboxylate (600 mg, 2.20 mmol) and Ag₂O (5.09 g, 22.00 mmol) in DCE (30 mL) was added iodomethane-d₃ (1.59 g, 11.00 mmol) dropwise at room temperature under a nitrogen atmosphere. The resulting mixture was stirred for 12 hours at 80° C. under nitrogen. The mixture was cooled to room temperature and filtered. The filter cake was washed with CH₂Cl₂ (3×100 mL). The filtrate was concentrated under reduced pressure. The residue was purified by silica gel column chromatography, eluting with petroleum ether/EtOAc (0% to 50% of EtOAc over 30 min) to afford ethyl (3S,4R)-3-((tert-butoxycarbonyl)amino)-4-(methoxy-d3)cyclopentane-1-carboxylate (400 mg, 1.30 mmol) as a light yellow solid. ¹H NMR (400 MHz, DMSO-d6) δ 6.49 (d, J=8.2 Hz, 1H), 4.05 (q, J=7.1 Hz, 2H), 3.91-3.80 (m, 1H), 3.68-3.61 (m, 1H), 2.90-2.76 (m, 1H), 2.04-1.90 (m, 2H), 1.79-1.72 (m, 2H), 1.38 (s, 9H), 1.17 (t, J=7.1 Hz, 3H).

### Step 2. Synthesis of (3S,4R)-3-((tert-butoxycarbonyl)amino)-4-(methoxy-d3)cyclopentane-1-carboxylic acid

To a stirred solution of ethyl (3S,4R)-3-((tert-butoxycarbonyl)amino)-4-(methoxy-d3)cyclopentane-1-carboxylate (290 mg, 999 μmol) in THF (3 mL) and H₂O (1 mL) was added LiOH (71 mg, 3.00 mmol) at room temperature under a nitrogen atmosphere. The resulting mixture was stirred for 4 hours at 30° C. under nitrogen. The mixture was acidified to pH 5 with HCl (1 N, aq.). The resulting mixture was extracted with EtOAc (3×100 mL). The combined organic layers were washed with brine (3×50 mL), and dried over anhydrous Na₂SO₄. After filtration, the filtrate was concentrated under reduced pressure. The crude product (3S,4R)-3-((tert-butoxycarbonyl)amino)-4-(methoxy-d3)cyclopentane-1-carboxylic acid (200 mg, 762 mol) was used in the next step directly without purification. LCMS RT 0.539 min, [M+H]⁺=263.1, LCMS method G.

### Step 3. Synthesis of tert-butyl ((1S,2R)-4-(((S)-(3-chloro-2,6-difluorophenyl)(4-fluorobicyclo[2.2.1]heptan-1-yl)methyl)carbamoyl)-2-(methoxy-d3)cyclopentyl)carbamate

To a stirred solution of (3S,4R)-3-((tert-butoxycarbonyl)amino)-4-(methoxy-d3)cyclopentane-1-carboxylic acid (80 mg, 0.30 mmol) and (S)-(3-chloro-2,6-difluorophenyl)(4-fluorobicyclo[2.2.1]heptan-1-yl)methanamine (88 mg, 0.30 mmol) in DMF (2 mL) was added sodium bicarbonate (77 mg, 0.91 mmol) and HATU (170 mg, 0.46 mmol) at room temperature under a nitrogen atmosphere. The resulting mixture was stirred for 3 hours at 30° C. under nitrogen. After concentration in vacuo, the residue was purified by reversed-phase flash chromatography (column: C18 gel; mobile phase A: water (0.1% NH₄OH), mobile phase B: acetonitrile; gradient: 10% to 90% B in 40 min; detector: UV 254/220 nm) to give tert-butyl ((1S,2R)-4-(((S)-(3-chloro-2,6-difluorophenyl)(4-fluorobicyclo[2.2.1]heptan-1-yl)methyl)carbamoyl)-2-(methoxy-d3)cyclopentyl)carbamate (80 mg, 0.14 mmol) as a white solid. LCMS RT 1.291 min, m/z [M−H]⁻ 532.2, LCMS method G.

### Step 4. Synthesis of (3S,4R)-3-amino-N—((S)-(3-chloro-2,6-difluorophenyl)(4-fluorobicyclo[2.2.1]heptan-1-yl)methyl)-4-(methoxy-d3)cyclopentane-1-carboxamide hydrochloride

To a stirred solution of tert-butyl ((1S,2R)-4-(((S)-(3-chloro-2,6-difluorophenyl)(4-fluorobicyclo[2.2.1]heptan-1-yl)methyl)carbamoyl)-2-(methoxy-d3)cyclopentyl)carbamate (40 mg, 75.00 μmol) in 1,4-dioxane (1 mL) was added HCl in 1,4-dioxane (4M, 1 mL) dropwise at room temperature under a nitrogen atmosphere. The resulting mixture was stirred for 2 hours at 30° C. under nitrogen. The mixture was concentrated and triturated with Et₂O (2 mL) twice. The crude product (3S,4R)-3-amino-N—((S)-(3-chloro-2,6-difluorophenyl)(4-fluorobicyclo[2.2.1]heptan-1-yl)methyl)-4-(methoxy-d3)cyclopentane-1-carboxamide hydrochloride (40 mg, crude) was used in the next step directly without further purification. LCMS RT 0.985 min, [M+H]⁺ 434.2, LCMS method G.

### Step 5. Synthesis of N-((1S,2R,4S)-4-(((S)-(3-chloro-2,6-difluorophenyl)(4-fluorobicyclo[2.2.1]heptan-1-yl)methyl)carbamoyl)-2-(methoxy-d3)cyclopentyl)pyrimidine-5-carboxamide and N-((1S,2R,4R)-4-(((S)-(3-chloro-2,6-difluorophenyl)(4-fluorobicyclo[2.2.1]heptan-1-yl)methyl)carbamoyl)-2-(methoxy-d3)cyclopentyl)pyrimidine-5-carboxamide

To a stirred solution of (3S,4R)-3-amino-N—((S)-(3-chloro-2,6-difluorophenyl)(4-fluorobicyclo[2.2.1]heptan-1-yl)methyl)-4-(methoxy-d3)cyclopentane-1-carboxamide hydrochloride (30 mg, 69 μmol) and pyrimidine-5-carboxylic acid (9 mg, 69 μmol) in DMF (1 mL) was added sodium bicarbonate (17 mg, 0.21 mmol) and HATU (39 mg, 0.10 mmol) at room temperature under a nitrogen atmosphere. The resulting mixture was stirred for 3 hours at 30° C. under nitrogen. After concentration under reduced pressure, the residue was purified by reversed-phase flash chromatography (column: C18 gel; mobile phase A: water (0.1% NH₄OH), mobile phase B: acetonitrile; gradient: 10% to 90% B in 40 min; detector: UV 254/220 nm) to give N-((1S,2R)-4-(((S)-(3-chloro-2,6-difluorophenyl)(4-fluorobicyclo[2.2.1]heptan-1-yl)methyl)carbamoyl)-2-(methoxy-d3)cyclopentyl)pyrimidine-5-carboxamide (20 mg, 51%) as a white solid. LCMS RT 1.177 min, [M+H]⁺ 540.2, LCMS method. It was further purified by preparative chiral HPLC with the following conditions (column: CHIRALPAK IA, 2*25 cm, 5 μm; mobile phase A: hexane (0.5% of 2M NH₃ in MeOH), mobile phase B: EtOH; flow rate: 20 mL/min; gradient: 50% B isocratic; wavelength: 200/215 nm; RT1 (min): 6.3; RT2 (min): 17.48; sample solvent: EtOH:CH₂Cl₂ 1:1; injection volume: 1 mL) to give N-((1 S,2R,4S)-4-(((S)-(3-chloro-2,6-difluorophenyl)(4-fluorobicyclo[2.2.1]heptan-1-yl)methyl)carbamoyl)-2-(methoxy-d3)cyclopentyl)pyrimidine-5-carboxamide and N-((1S,2R,4R)-4-(((S)-(3-chloro-2,6-difluorophenyl)(4-fluorobicyclo[2.2.1]heptan-1-yl)methyl)carbamoyl)-2-(methoxy-d3)cyclopentyl)pyrimidine-5-carboxamide, both as a white solid. Isomer 1: 7.1 mg, 13 μmol, LCMS RT 1.875 min, [M+H]⁺ 540.25, LCMS method F, ¹H NMR (400 MHz, DMSO-d6) δ 9.30 (d, J=3.2 Hz, 1H), 9.17-9.08 (m, 2H), 8.57 (d, J=7.7 Hz, 1H), 8.28 (d, J=8.1 Hz, 1H), 7.58 (td, J=8.7, 5.4 Hz, 1H), 7.16 (t, J=9.5 Hz, 1H), 5.30 (d, J=8.2 Hz, 1H), 4.30 (dt, J=12.6, 6.2 Hz, 1H), 3.77 (s, 1H), 3.16-3.02 (m, 1H), 2.07-1.93 (m, 3H), 1.86-1.56 (m, 9H), 1.46 (s, 2H). ¹⁹F NMR (282 MHz, DMSO) δ −111.047, −113.597, −173.563. Isomer 2: 3.0 mg, 5.5 μmol, LCMS RT 1.875 min, [M+H]⁺ 540.25, LCMS method F. ¹H NMR (400 MHz, DMSO-d6) δ 9.30 (s, 1H), 9.14 (d, J=1.4 Hz, 2H), 8.56 (d, J=8.0 Hz, 1H), 8.28 (d, J=8.3 Hz, 1H), 7.57 (td, J=8.7, 5.5 Hz, 1H), 7.23-7.09 (m, 1H), 5.29 (d, J=8.2 Hz, 1H), 4.35-4.18 (m, 1H), 3.80 (td, J=4.5, 2.4 Hz, 1H), 3.10 (d, J=10.5 Hz, 1H), 2.12 (ddd, J=13.6, 8.8, 2.5 Hz, 1H), 1.94 (q, J=10.5 Hz, 1H), 1.85-1.68 (m, 9H), 1.61 (d, J=8.4 Hz, 1H), 1.47 (d, J=8.0 Hz, 2H). ¹⁹F NMR (282 MHz, DMSO) δ −111.635, −113.401, −173.565.

### Example 22

### (5R,7R)—N—((R)-(2,3-dichloro-6-fluorophenyl)(1-methylcyclopentyl)methyl)-3-methyl-2-oxo-1,3-diazaspiro[4.4]nonane-7-carboxamide, (5R,7S)—N—((R)-(2,3-dichloro-6-fluorophenyl)(1-methylcyclopentyl)methyl)-3-methyl-2-oxo-1,3-diazaspiro[4.4]nonane-7-carboxamide, (5S,7S)—N—((R)-(2,3-dichloro-6-fluorophenyl)(1-methylcyclopentyl)methyl)-3-methyl-2-oxo-1,3-diazaspiro[4.4]nonane-7-carboxamide and (5S,7R)—N—((R)-(2,3-dichloro-6-fluorophenyl)(1-methylcyclopentyl)methyl)-3-methyl-2-oxo-1,3-diazaspiro[4.4]nonane-7-carboxamide

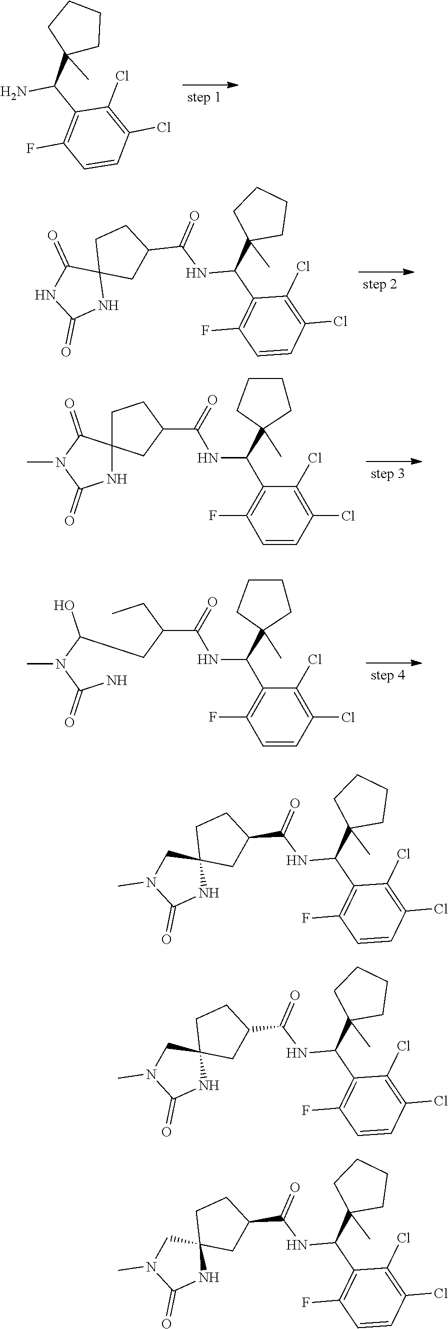

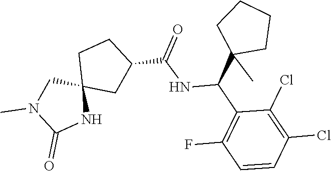 [SMILES: CN1C[C@@]2(CC[C@H](C(=O)N[C@H](c3c(F)ccc(Cl)c3Cl)C3(C)CCCC3)C2)NC1=O]

### Step 1. Synthesis of N—((S)-(2,3-dichloro-6-fluorophenyl)(1-methylcyclopentyl)methyl)-2,4-dioxo-1,3-diazaspiro[4.4]nonane-7-carboxamide

To a mixture of 2,4-dioxo-1,3-diazaspiro [4.4]nonane-7-carboxylic acid (300 mg, 1.51 mmol), (S)-(2,3-dichloro-6-fluorophenyl)(1-methylcyclopentyl)methanamine (418 mg, 1.51 mmol) and TEA (917 mg, 9.08 mmol) in DMF (3 mL) was added T₃P (1.93 g, 50% wt, 3.03 mmol). The mixture was stirred for 2 hours at 25° C. The reaction mixture was diluted with water (20 mL), and the aqueous phase was extracted with ethyl acetate (20 mL) three times. The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 0% to 100% B in 25 min; detector: UV 254 nm). Concentration in vacuo resulted in N—((S)-(2,3-dichloro-6-fluorophenyl)(1-methylcyclopentyl)methyl)-2,4-dioxo-1,3-diazaspiro[4.4]nonane-7-carboxamide (0.23 g, 33%) as a colorless oil. LCMS RT 0.981 min, [M+H]⁺ 456, LC method C.

### Step 2. Synthesis of N—((S)-(2,3-dichloro-6-fluorophenyl)(1-methylcyclopentyl)methyl)-3-methyl-2,4-dioxo-1,3-diazaspiro[4.4]nonane-7-carboxamide

A mixture of N—((S)-(2,3-dichloro-6-fluorophenyl)(1-methylcyclopentyl)methyl)-2,4-dioxo-1,3-diazaspiro[4.4]nonane-7-carboxamide (220 mg, 482 μmol) and 1,1-dimethoxy-N, N-dimethylethan-1-amine (193 mg, 1.45 mmol) in toluene (2 mL) was stirred for 2 hours at 110° C. The reaction mixture was concentrated in vacuo. The residue was purified by reverse phase flash chromatography (column: C18 silica gel; mobile phase A: water, mobile phase B: acetonitrile; gradient: 0% to 100% B in 25 min; detector: UV 254 nm) to give N—((S)-(2,3-dichloro-6-fluorophenyl)(1-methylcyclopentyl)methyl)-3-methyl-2,4-dioxo-1,3-diazaspiro[4.4]nonane-7-carboxamide (0.19 g, 0.40 mmol) as a colorless oil. LCMS RT 0.994 min, [M+H]⁺ 470, LCMS method C.

### Step 3. Synthesis of N—((S)-(2,3-dichloro-6-fluorophenyl)(1-methylcyclopentyl)methyl)-4-hydroxy-3-methyl-2-oxo-1,3-diazaspiro[4.4]nonane-7-carboxamide

To a mixture of N—((S)-(2,3-dichloro-6-fluorophenyl)(1-methylcyclopentyl)methyl)-3-methyl-2,4-dioxo-1,3-diazaspiro[4.4]nonane-7-carboxamide (180 mg, 383 μmol) in THF (3 mL) was added LiAlH₄ (22 mg, 574 μmol) in portions at 0° C. under a nitrogen atmosphere. The mixture was stirred for 2 hours at 25° C. The reaction was then cooled to 0° C. and quenched with water (0.18 mL), sodium hydroxide (0.36 mL, 4M) and then water (0.18 mL). The mixture was filtered through a pad of Celite. The pad was washed with ethyl acetate, and the filtrate was concentrated in vacuo. The residue was purified by reverse phase flash chromatography (column: C18 silica gel; mobile phase A: water, mobile phase B: acetonitrile; gradient: 0% to 100% in 25 min; detector: UV 254 nm) to give N—((S)-(2,3-dichloro-6-fluorophenyl)(1-methylcyclopentyl)methyl)-3-methyl-2,4-dioxo-1,3-diazaspiro[4.4]nonane-7-carboxamide (0.15 g, 0.32 mmol) as a colorless oil. LCMS RT 0.941 min, [M+H]⁺ 472, LCMS method C.

### Step 4. Synthesis of (5R,7R)—N—((R)-(2,3-dichloro-6-fluorophenyl)(1-methylcyclopentyl)methyl)-3-methyl-2-oxo-1,3-diazaspiro[4.4]nonane-7-carboxamide, (5R,7S)—N—((R)-(2,3-dichloro-6-fluorophenyl)(1-methylcyclopentyl)methyl)-3-methyl-2-oxo-1,3-diazaspiro[4.4]nonane-7-carboxamide, (5S,7S)—N—((R)-(2,3-dichloro-6-fluorophenyl)(1-methylcyclopentyl)methyl)-3-methyl-2-oxo-1,3-diazaspiro[4.4]nonane-7-carboxamide and (5S,7R)—N—((R)-(2,3-dichloro-6-fluorophenyl)(1-methylcyclopentyl)methyl)-3-methyl-2-oxo-1,3-diazaspiro[4.4]nonane-7-carboxamide

To a mixture of N—((S)-(2,3-dichloro-6-fluorophenyl)(1-methylcyclopentyl)methyl)-3-methyl-2,4-dioxo-1,3-diazaspiro[4.4]nonane-7-carboxamide (140 mg, 335 μmol) in THF (2 mL) was added Et₃SiH (78.3 mg, 669 μmol) dropwise at room temperature, and then TFA (76.3 mg, 669 μmol) was added. The mixture was stirred for 2 hours at 70° C. The reaction mixture was concentrated in vacuo. The resulting crude material was purified by preparative HPLC (mobile phase A: water with 0.10% formic acid, mobile phase B: acetonitrile) to give N—((S)-(2,3-dichloro-6-fluorophenyl)(1-methylcyclopentyl)methyl)-3-methyl-2-oxo-1,3-diazaspiro[4.4]nonane-7-carboxamide (0.10 g) as a colorless oil. LCMS RT 1.020 min, [M+H]⁺ 456, LCMS method C.

The product was further purified by chiral preparative HPLC (column: CHIRALPAK IG, 2*25 cm, 5 μm; mobile phase A: hexane, mobile phase B: EtOH; flow rate: 20 mL/min; gradient: 15% B isocratic; wavelength: 220/254 nm; RT1 (min): 9.259; RT2 (min): 11.358; sample solvent: EtOH:DCM 1:1; injection volume: 1.5 mL) to (5R,7R)—N—((R)-(2,3-dichloro-6-fluorophenyl)(1-methylcyclopentyl)methyl)-3-methyl-2-oxo-1,3-diazaspiro[4.4]nonane-7-carboxamide, (5R,7S)—N—((R)-(2,3-dichloro-6-fluorophenyl)(1-methylcyclopentyl)methyl)-3-methyl-2-oxo-1,3-diazaspiro[4.4]nonane-7-carboxamide, (5S,7S)—N—((R)-(2,3-dichloro-6-fluorophenyl)(1-methylcyclopentyl)methyl)-3-methyl-2-oxo-1,3-diazaspiro[4.4]nonane-7-carboxamide and (5S,7R)—N—((R)-(2,3-dichloro-6-fluorophenyl)(1-methylcyclopentyl)methyl)-3-methyl-2-oxo-1,3-diazaspiro[4.4]nonane-7-carboxamide, all as an off-white amorphous solid.

Isomer 1: 6.8 mg, 15 μmol. ¹H NMR (400 MHz, DMSO-d6) δ 8.12 (d, J=8.6 Hz, 1H), 7.61 (dd, J=9.0, 5.1 Hz, 1H), 7.26 (dd, J=10.8, 9.0 Hz, 1H), 6.63 (s, 1H), 5.48 (d, J=8.5 Hz, 1H), 3.17-3.06 (m, 2H), 3.06-2.98 (m, 1H), 2.56 (s, 3H), 1.92 (td, J=9.0, 8.5, 5.6 Hz, 1H), 1.82-1.54 (m, 4H), 1.60 (s, 7H), 1.37 (s, 1H), 1.26 (t, J=9.2 Hz, 1H), 0.97 (d, J=2.8 Hz, 3H), LCMS RT 1.205 min, [M+H]⁺ 456.10, LC method B

Isomer 2: 7.4 mg, 16 μmol. ¹H NMR (400 MHz, DMSO-d6) δ 8.11 (d, J=8.6 Hz, 1H), 7.61 (dd, J=8.9, 5.1 Hz, 1H), 7.26 (dd, J=10.7, 9.0 Hz, 1H), 6.65 (s, 1H), 5.48 (d, J=8.5 Hz, 1H), 3.22 (d, J=8.8 Hz, 1H), 3.12 (d, J=8.8 Hz, 1H), 3.02 (p, J=7.7 Hz, 1H), 2.58 (s, 3H), 1.89 (dd, J=13.2, 9.3 Hz, 1H), 1.78 (dt, J=13.0, 5.9 Hz, 2H), 1.72-1.44 (m, 9H), 1.38 (d, J=6.7 Hz, 1H), 1.31-1.22 (m, 1H), 0.97 (d, J=2.8 Hz, 3H), LCMS RT 1.195 min, [M+H]⁺ 456.10, LCMS method B

Isomer 3: 27.4 mg, 60.0 μmol. ¹H NMR (400 MHz, DMSO-d6) δ 8.08 (d, J=8.7 Hz, 1H), 7.61 (dd, J=8.9, 5.0 Hz, 1H), 7.31-7.21 (m, 1H), 6.45 (s, 1H), 5.50 (d, J=8.5 Hz, 1H), 3.14 (q, J=8.5 Hz, 2H), 2.94 (p, J=8.2 Hz, 1H), 2.51 (p, J=1.8 Hz, 3H), 1.85-1.71 (m, 3H), 1.71-1.50 (m, 9H), 1.41-1.33 (m, 1H), 1.27 (d, J=8.1 Hz, 1H), 0.97 (d, J=2.8 Hz, 3H), LCMS RT 1.210 min, [M+H]+, 456.10, LCMS method B

Isomer 4: 27.4 mg, 60.0 μmol. ¹H NMR (400 MHz, DMSO-d6) δ 8.09 (d, J=8.7 Hz, 1H), 7.62 (dd, J=8.9, 5.1 Hz, 1H), 7.26 (dd, J=10.7, 8.9 Hz, 1H), 6.49 (s, 1H), 5.52 (d, J=8.6 Hz, 1H), 3.19 (d, J=8.6 Hz, 1H), 3.13 (d, J=8.5 Hz, 1H), 2.98-2.90 (m, 1H), 2.61 (s, 3H), 1.89 (dd, J=12.4, 7.8 Hz, 1H), 1.79-1.49 (m, 1OH), 1.40-1.33 (m, 1H), 1.27 (d, J=8.1 Hz, 1H), 0.96 (d, J=2.8 Hz, 3H), LCMS RT 1.198 min, [M+H]⁺ 456.10, LCMS method B.

### Example 23

### (2r,4S)—N—((S)-(3-chloro-2,6-difluorophenyl)(cyclopentyl)methyl)-5-(2-hydroxyethyl)-6,8-dioxo-5,7-diazaspiro[3.4]octane-2-carboxamide

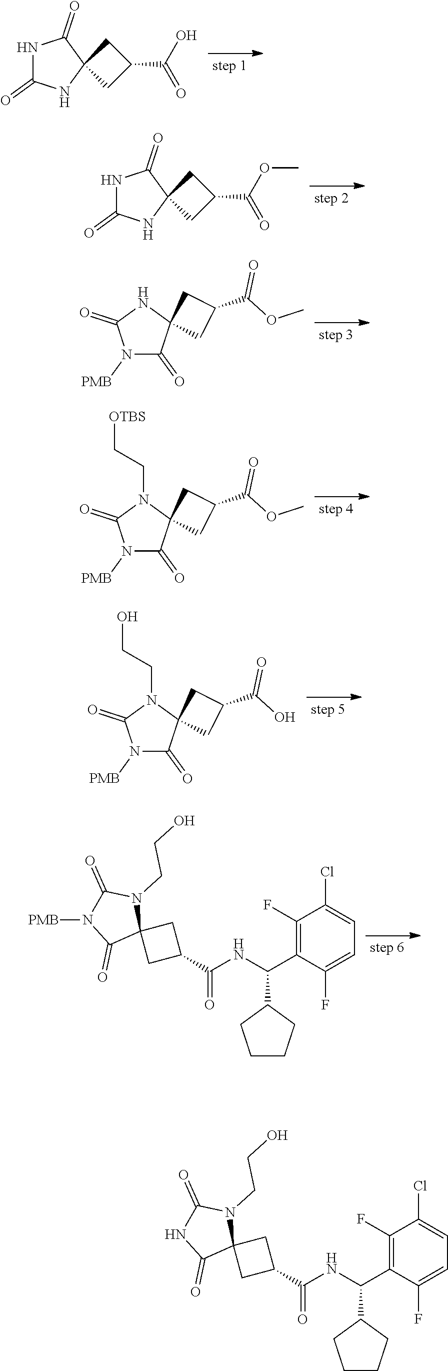

### Step 1. Synthesis of methyl (2r,4r)-6,8-dioxo-5,7-diazaspiro[3.4]octane-2-carboxylate

To a mixture of (2r,4r)-6,8-dioxo-5,7-diazaspiro[3.4]octane-2-carboxylic acid (1 g, 5 mmol) in DCM/MeOH (2:1, 10 mL) was added TMSCHN₂ (8 mL, 2 M, 16 mmol) dropwise at 0° C. under a nitrogen atmosphere. The mixture was stirred for 2 h at room temperature. The reaction was quenched with saturated NH₄Cl (aq.) and the aqueous phase was extracted with DCM (20 mL) three times. The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 5% to 40% B in 10 min; detector: UV 220 nm) to afford methyl (2r,4r)-6,8-dioxo-5,7-diazaspiro[3.4]octane-2-carboxylate (500 mg, 2.52 mmol) as a colorless oil. LCMS RT 0.535 min, [M+H]⁺ 199, LCMS method C.

### Step 2. Synthesis of methyl (2s,4s)-7-(4-methoxybenzyl)-6,8-dioxo-5,7-diazaspiro[3.4]octane-2-carboxylate

To a mixture of methyl (2r,4r)-6,8-dioxo-5,7-diazaspiro[3.4]octane-2-carboxylate (1.7 g, 8.6 mmol), Cs₂CO₃ (5.6 g, 17 mmol) in DMF (20 mL) was added 1-(chloromethyl)-4-m ethoxybenzene (1.5 g, 9.4 mmol) dropwise at 0° C. under a nitrogen atmosphere. The mixture was stirred for 16 hours at 0° C. The reaction mixture was diluted with water (100 mL), and the aqueous phase was extracted with ethyl acetate (100 mL) three times. The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 0% to 100% B in 25 min; detector: UV 254 nm) to give methyl (2s,4s)-7-(4-methoxybenzyl)-6,8-dioxo-5,7-diazaspiro [3.4]octane-2-carboxylate (1 g, 3 mmol) as a colorless oil. LCMS RT 0.696 min, [M+H]⁺ 319, LCMS method A.

### Step 3. Synthesis of methyl (2s,4s)-5-(2-((tert-butyldimethylsilyl)oxy)ethyl)-7-(4-methoxybenzyl)-6,8-dioxo-5,7-diazaspiro[3.4]octane-2-carboxylate

To a mixture of methyl (2s,4s)-7-(4-methoxybenzyl)-6,8-dioxo-5,7-diazaspiro[3.4]octane-2-carboxylate (290 mg, 911 μmol) and Cs₂CO₃ (594 mg, 1.82 mmol) in DMF (5 mL) was added (2-bromoethoxy)(tert-butyl)dimethylsilane (262 mg, 1.09 mmol) at −78° C. The mixture was stirred for 2 h at room temperature. The reaction mixture was diluted with water (10 ml) and extracted with ethyl acetate (50 mL*3). The combined organic layers were washed with brine, dried over sodium sulfate, filtered and concentrated in vacuo. The resulting crude material was purified by C18 flash (acetonitrile/water) to give methyl (2s,4s)-5-(2-((tert-butyldimethylsilyl)oxy)ethyl)-7-(4-methoxybenzyl)-6,8-dioxo-5,7-diazaspiro[3.4]octane-2-carboxylate (275 mg, 577 μmol) as a colorless oil. LCMS RT 1.456 min, [M+H]⁺ 477, LCMS method C.

### Step 4. Synthesis of (2s,4s)-5-(2-hydroxyethyl)-7-(4-methoxybenzyl)-6,8-dioxo-5,7-diazaspiro[3.4]octane-2-carboxylic acid

A mixture of methyl (2s,4s)-5-{2-[(tert-butyldimethylsilyl)oxy]ethyl}-7-[(4-methoxyphenyl)methyl]-6,8-dioxo-5,7-diazaspiro[3.4]octane-2-carboxylate (1 g, 2.098 mmol) and NaOH (0.25 g, 6.294 mmol) in MeOH (10 mL) was stirred for 1 h at 25° C. The mixture was acidified to pH 5 with HCl (1N). The precipitated solids were collected by filtration and washed with MeOH to give (2s,4s)-5-(2-hydroxyethyl)-7-[(4-methoxyphenyl)methyl]-6,8-dioxo-5,7-diazaspiro[3.4]octane-2-carboxylic acid (530 mg,) as an off-white solid. LCMS RT 0.640 min, [M+H]⁺ 349, LCMS method C.

### Step 5. Synthesis of (2r,4S)—N—((S)-(3-chloro-2,6-difluorophenyl)(cyclopentyl)methyl)-5-(2-hydroxyethyl)-7-(4-methoxybenzyl)-6,8-dioxo-5,7-diazaspiro[3.4]octane-2-carboxamide

A mixture of (2s,4s)-5-(2-hydroxyethyl)-7-[(4-methoxyphenyl)methyl]-6,8-dioxo-5,7-diazaspiro[3.4]octane-2-carboxylic acid (530 mg, 1.521 mmol), (1S)-1-(3-chloro-2,6-difluorophenyl)-1-cyclopentylmethanamine (373.82 mg, 1.521 mmol), T₃P (726.14 mg, 2.281 mmol) and TEA (461.88 mg, 4.563 mmol) in DCM (8 mL) was stirred for 1 h at 25° C. The reaction mixture was diluted with water (10 mL), and the aqueous phase was extracted with DCM (10 mL) three times. The combined organic layers were washed with brine, dried over sodium sulfate, filtered and concentrated in vacuo. The residue was purified by reverse phase flash chromatography (column, C18 silica gel; mobile phase A: water, mobile phase B: acetonitrile; gradient: 0% to 100% B in 10 min; detector: UV 220 nm) to give (2r,4S)—N—((S)-(3-chloro-2,6-difluorophenyl)(cyclopentyl)methyl)-5-(2-hydroxyethyl)-7-(4-methoxybenzyl)-6,8-dioxo-5,7-diazaspiro[3.4]octane-2-carboxamide (540 mg) as an off-white solid. LCMS RT 1.227 min, [M+H]⁺ 576, LCMS method C.

### Step 6. Synthesis of (2r,4S)—N—((S)-(3-chloro-2,6-difluorophenyl)(cyclopentyl)methyl)-5-(2-hydroxyethyl)-6,8-dioxo-5,7-diazaspiro[3.4]octane-2-carboxamide

A mixture of (2r,4S)—N—((S)-(3-chloro-2,6-difluorophenyl)(cyclopentyl)methyl)-5-(2-hydroxyethyl)-7-(4-methoxybenzyl)-6,8-dioxo-5,7-diazaspiro[3.4]octane-2-carboxamide (540 mg, 0.937 mmol) and Ce(NH₄)₂(NO₃)₆ (515.81 mg, 0.937 mmol) in acetonitrile/H₂O (10 mL, 4:1) was stirred for 1 h at 70° C. The reaction mixture was diluted with water (20 mL), and the aqueous phase was extracted with ethyl acetate (20 mL) three times. The combined organic layers were washed with brine, dried over sodium sulfate, filtered and concentrated in vacuo. The residue was purified by reverse phase flash chromatography (column, C18 silica gel; mobile phase A: water, mobile phase B: acetonitrile; gradient: 0% to 100% B in 10 min; detector: UV 220 nm) to give (2r,4S)—N—((S)-(3-chloro-2,6-difluorophenyl)(cyclopentyl)methyl)-5-(2-hydroxyethyl)-6,8-dioxo-5,7-diazaspiro[3.4]octane-2-carboxamide (10 mg) as an off-white solid. ¹H NMR (400 MHz, DMSO-d6) δ 10.62 (s, 1H), 8.38 (d, J=7.4 Hz, 1H), 7.53 (td, J=8.6, 5.4 Hz, 1H), 7.21-7.03 (m, 1H), 4.97-4.72 (m, 2H), 3.61 (q, J=5.9 Hz, 2H), 3.42 (t, J=6.1 Hz, 2H), 3.19 (q, J=9.3 Hz, 1H), 2.62 (ddd, J=21.6, 12.6, 8.7 Hz, 3H), 2.47-2.33 (m, 2H), 1.88 (dt, J=12.4, 5.1 Hz, 1H), 1.69-1.42 (m, 4H), 1.32 (ddd, J=26.8, 12.2, 6.2 Hz, 2H), 1.02 (d, J=9.8 Hz, 1H). LCMS RT 1.078 min, [M+H]⁺ 456.10, LCMS method B.

### Example 24

### (1R,3R)-3-acetamido-N—((S)-(2,3-dichloro-6-fluorophenyl)(1-(2-hydroxyethyl)cyclopentyl)methyl)cyclopentane-1-carboxamide

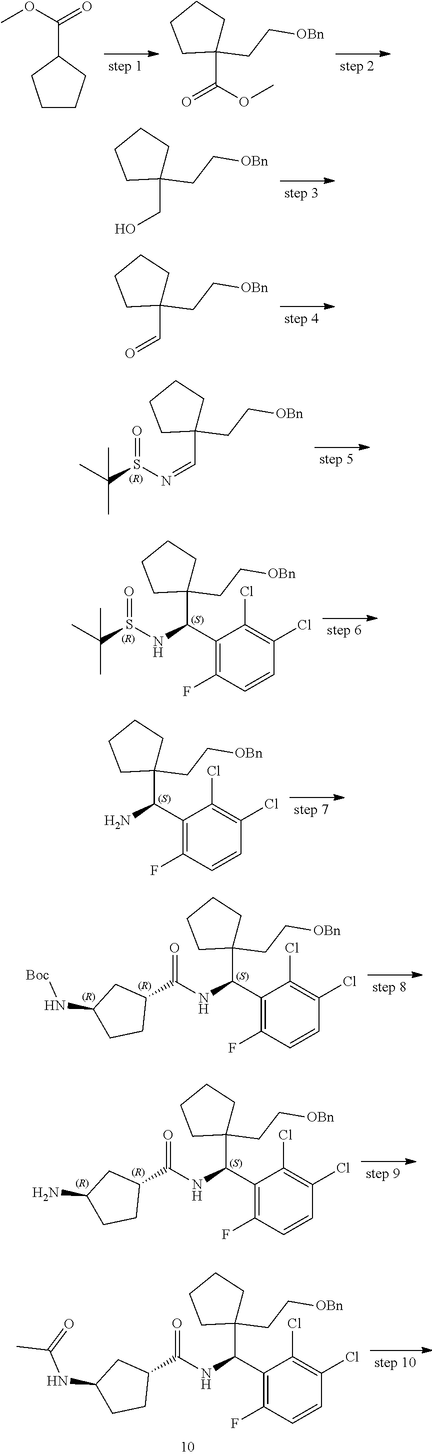

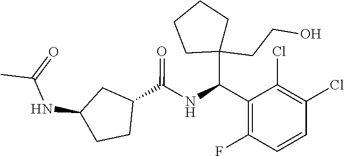 [SMILES: CC(=O)N[C@@H]1CC[C@@H](C(=O)N[C@H](c2c(F)ccc(Cl)c2Cl)C2(CCO)CCCC2)C1]

### Step 1. Synthesis of methyl 1-(2-(benzyloxy)ethyl)cyclopentane-1-carboxylate

To a mixture of methyl cyclopentanecarboxylate (5 g, 0.04 mol) in THF (70 mL) was added LDA (30 mL, 2 molar, 0.06 mol) dropwise at −78° C. under a nitrogen atmosphere. The mixture was stirred for 1 h at −78° C. prior to the addition of ((2-bromoethoxy) methyl) benzene (10 g, 0.05 mol) dropwise at −78° C. The mixture was stirred for 16 h at room temperature. The reaction was quenched with saturated NH₄Cl (aq.) and the aqueous phase was extracted with ethyl acetate (250 mL) three times. The combined organic layers were washed with brine, dried over sodium sulfate, filtered and concentrated in vacuo. The resulting crude material was purified by C18 flash chromatography (mobile phase A: water, mobile phase B: acetonitrile) to give methyl 1-(2-(benzyloxy) ethyl) cyclopentane-1-carboxylate (7.2 g, 27 mmol) as a colorless oil. LCMS RT 1.123 min, [M+H]⁺ 263, LCMS method C.

### Step 2. Synthesis of (1-(2-(benzyloxy)ethyl)cyclopentyl)methanol

To a mixture of methyl 1-(2-(benzyloxy) ethyl) cyclopentane-1-carboxylate (7.1 g, 27 mmol) in THF (100 mL) was added LiAlH₄ (1.2 g, 32 mmol) in portions at 0° C. The mixture was stirred for 2 h at room temperature. The reaction was cooled to 0° C. and quenched with water (1.5 mL), sodium hydroxide (3 mL, 4N) and water (1.5 mL). The mixture was filtered through a pad of Celite. The pad was washed with DCM, and the filtrate was concentrated in vacuo resulted in (1-(2-(benzyloxy)ethyl)cyclopentyl)methanol (5 g, 0.02 mol) as a colorless oil. LCMS RT 0.988 min, [M+H]⁻ 235, LCMS method C.

### Step 3. Synthesis of 1-(2-(benzyloxy)ethyl)cyclopentane-1-carbaldehyde

To a mixture of (1-(2-(benzyloxy)ethyl)cyclopentyl)methanol (4.9 g, 21 mmol) and molecule sieve 4 Å activated powder (500 mg) in DCM (100 mL) was added PCC (5.4 g, 25 mmol) at 0° C. The mixture was stirred at 0° C. for 2 h. The mixture was diluted with ether/pentane (1:1, 500 mL). The mixture was then filtered through Celite (50 g). The pad was washed with ether. The combined filtrate was concentrated (water bath temperature <15° C.) to ˜2 mL to give 1-(2-(benzyloxy)ethyl)cyclopentane-1-carbaldehyde (5 g, 0.02 mol, crude). LCMS RT 1.107 min, [M+Na]+255, LCMS method C.

### Step 4. Synthesis of (R)—N-((1-(2-(benzyloxy)ethyl)cyclopentyl)methylene)-2-methylpropane-2-sulfinamide

A mixture of 1-(2-(benzyloxy) ethyl) cyclopentane-1-carbaldehyde (5.5 g, 24 mmol), (R)-2-methylpropane-2-sulfinamide (3.2 g, 26 mmol) and Ti(OⁱPr)₄ (6.7 g, 24 mmol) in THF (100 mL) was stirred for 2 h at 50° C. The reaction mixture was diluted with water (300 mL) and filtrated. The filtrate was extracted with ethyl acetate (350 mL) three times. The combined organic layers were washed with brine, dried over sodium sulfate, filtered and concentrated in vacuo. The resulting crude material was purified by C18 flash chromatography (acetonitrile/water) to give (R)—N-((1-(2-(benzyloxy)ethyl)cyclopentyl)methylene)-2-methylpropane-2-sulfinamide (2.9 g, 8.6 mmol) as a colorless oil. LCMS RT1.210 min, [M+H]⁺ 336, LCMS method C.

### Step 5. Synthesis of (R)—N—((S)-(1-(2-(benzyloxy)ethyl)cyclopentyl)(2,3-dichloro-6-fluorophenyl)methyl)-2-methylpropane-2-sulfinamide

To a mixture of 1,2-dichloro-4-fluorobenzene (1.7 g, 10 mmol) in THF (50 mL) was added LDA (6.3 mL, 2 molar, 13 mmol) dropwise at −78° C. under a nitrogen atmosphere. The mixture was stirred for 1 h at −78° C. prior to the addition of (R)—N-((1-(2-(benzyloxy)ethyl)cyclopentyl)methylene)-2-methylpropane-2-sulfinamide (2.8 g, 8.3 mmol) at −78° C. The mixture was stirred for 16 h at room temperature. The reaction was quenched with saturated NH₄Cl (aq.) and the aqueous phase was extracted with ethyl acetate (300 mL) three times. The combined organic layers were washed with brine, dried over sodium sulfate, filtered and concentrated in vacuo. The resulting crude material was purified by C18 flash chromatography (acetonitrile/water) to give (R)—N—((S)-(1-(2-(benzyloxy)ethyl)cyclopentyl)(2,3-dichloro-6-fluorophenyl)methyl)-2-methylpropane-2-sulfinamide (2.5 g, 5.0 mmol) as a yellow oil. LCMS RT1.342 min, [M+H]⁺ 500, LCMS method C.

### Step 6. Synthesis of (S)-(1-(2-(benzyloxy)ethyl)cyclopentyl)(2,3-dichloro-6-fluorophenyl)methanamine

A mixture of (R)—N—((S)-(1-(2-(benzyloxy)ethyl)cyclopentyl)(2,3-dichloro-6-fluorophenyl)methyl)-2-methylpropane-2-sulfinamide (2.5 g, 5.0 mmol) in HCl (30 ml, 4 N in dioxane) was stirred for 1 h at room temperature. The mixture was concentrated in vacuo to afford (S)-(1-(2-(benzyloxy)ethyl)cyclopentyl)(2,3-dichloro-6-fluorophenyl)methanamine (1.9 g, 4.8 mmol) as a yellow oil. LCMS RT 0.896 min, [M+H]⁺ 396, LCMS method C.

### Step 7. Synthesis of tert-butyl ((1R,3R)-3-(((S)-(1-(2-(benzyloxy)ethyl)cyclopentyl)(2,3-dichloro-6-fluorophenyl)methyl)carbamoyl)cyclopentyl)carbamate

To a mixture of (S)-(1-(2-(benzyloxy)ethyl)cyclopentyl)(2,3-dichloro-6-fluorophenyl)methanamine (400 mg, 1.01 mmol), (1R,3R)-3-((tert-butoxycarbonyl)amino) cyclopentane-1-carboxylic acid (231 mg, 1.01 mmol), TEA (306 mg, 3.03 mmol) in DMF (8 mL) was added T₃P (642 mg, 2.02 mmol). The mixture was stirred for 1 h at room temperature. The reaction mixture was diluted with water (50 mL), and the aqueous phase was extracted with ethyl acetate (60 mL) three times. The combined organic layers were washed with brine, dried over sodium sulfate, filtered and concentrated in vacuo. The resulting crude material was purified by C18 flash chromatography (acetonitrile/water) to give tert-butyl ((1R,3R)-3-(((S)-(1-(2-(benzyloxy)ethyl)cyclopentyl)(2,3-dichloro-6-fluorophenyl)methyl)carbamoyl)cyclopentyl)carbamate (360 mg, 593 μmol) as a yellow oil. LCMS RT1.469 min, [M+H]⁺ 607, LCMS method C.

### Step 8. Synthesis of (1R,3R)-3-amino-N—((S)-(1-(2-(benzyloxy)ethyl)cyclopentyl)(2,3-dichloro-6-fluorophenyl)methyl)cyclopentane-1-carboxamide

A mixture of tert-butyl ((1R,3R)-3-(((S)-(1-(2-(benzyloxy)ethyl)cyclopentyl)(2,3-dichloro-6-fluorophenyl)methyl)carbamoyl)cyclopentyl)carbamate (340 mg, 560 μmol) in HCl (5 ml, 4 N in dioxane) was stirred for 1 h at room temperature. The mixture was concentrated in vacuo to afford (1R,3R)-3-amino-N—((S)-(1-(2-(benzyloxy)ethyl)cyclopentyl)(2,3-dichloro-6-fluorophenyl)methyl)cyclopentane-1-carboxamide (230 mg, 453 μmol) as a yellow oil. LCMS RT 0.921 min, [M+H]⁺ 507, LCMS method C.

### Step 9. Synthesis of (1R,3R)-3-acetamido-N—((S)-(1-(2-(benzyloxy)ethyl)cyclopentyl)(2,3-dichloro-6-fluorophenyl)methyl)cyclopentane-1-carboxamide

To a mixture of (1R,3R)-3-amino-N—((S)-(1-(2-(benzyloxy)ethyl)cyclopentyl)(2,3-dichloro-6-fluorophenyl)methyl)cyclopentane-1-carboxamide (200 mg, 394 μmol) and TEA (119 mg, 1.18 mmol) in DCM (5 mL) was added acetyl chloride (30.9 mg, 394 μmol) dropwise at 0° C. The solution was stirred for 1 h at room temperature. The reaction was quenched with water. The aqueous phase was extracted with ethyl acetate (50 mL) three times. The combined organic layers were washed with brine, dried over sodium sulfate, filtered and concentrated in vacuo. The resulting crude material was purified by C18 flash chromatography (acetonitrile/water) to give (1R,3R)-3-acetamido-N—((S)-(1-(2-(benzyloxy)ethyl)cyclopentyl)(2,3-dichloro-6-fluorophenyl)methyl)cyclopentane-1-carboxamide (190 mg, 346 μmol) as an off-white amorphous solid. LCMS RT 1.129 min, [M+H]⁺ 549, LCMS method C.

### Step 10. Synthesis of (1R,3R)-3-acetamido-N—((S)-(2,3-dichloro-6-fluorophenyl)(1-(2-hydroxyethyl)cyclopentyl)methyl)cyclopentane-1-carboxamide

A mixture of (1R,3R)-3-acetamido-N—((S)-(1-(2-(benzyloxy)ethyl)cyclopentyl)(2,3-dichloro-6-fluorophenyl)methyl)cyclopentane-1-carboxamide (100 mg, 182 μmol) and Ce(NH₄)₂(NO₃)₆ (997 mg, 1.82 mmol) in acetonitrile/H₂O (2:1, 10 mL) was stirred for 16 h at room temperature. The mixture was diluted with water. The mixture was extracted with ethyl acetate (100 mL) three times. The combined organic layers were washed with brine, dried over sodium sulfate, filtered and concentrated in vacuo. The resulting crude material was purified by preparative HPLC (column: Xselect CSH C18 OBD Column 30*150 mm 5 μm; mobile phase A: water (0.1% formic acid), mobile phase B: acetonitrile; flow rate: 60 mL/min; gradient: 36% B to 47% B in 7 min, then 47% B; wavelength: 254/220 nm; RT1 (min): 6.29) to give (1R,3R)-3-acetamido-N—((S)-(2,3-dichloro-6-fluorophenyl)(1-(2-hydroxyethyl)cyclopentyl)methyl)cyclopentane-1-carboxamide (16.3 mg, 35.5 μmol) as an off-white amorphous solid. ¹H NMR (400 MHz, DMSO-d6) δ 8.15 (d, J=8.6 Hz, 1H), 7.77 (d, J=7.0 Hz, 1H), 7.61 (dd, J=9.0, 5.0 Hz, 1H), 7.25 (dd, J=10.8, 8.9 Hz, 1H), 5.51 (d, J=8.5 Hz, 1H), 4.40 (t, J=4.7 Hz, 1H), 4.00 (q, J=6.5 Hz, 1H), 3.43 (s, 2H), 2.99-2.91 (m, 1H), 1.94-1.72 (m, 7H), 1.62 (dt, J=14.0, 8.2 Hz, 3H), 1.56-1.30 (m, 8H), 1.15 (t, J=10.6 Hz, 1H). LCMS RT 0.817 min, [M+H]⁺ 459, LCMS method C.

### Example 25

### (1S,3S,4S)-3-acetamido-N—((S)-(2,3-dichloro-6-fluoro-5-hydroxyphenyl)(4-fluorobicyclo[2.2.1]heptan-1-yl)methyl)-4-fluorocyclopentane-1-carboxamide and (1R,3R,4R)-3-acetamido-N—((S)-(2,3-dichloro-6-fluoro-5-hydroxyphenyl)(4-fluorobicyclo[2.2.1]heptan-1-yl)methyl)-4-fluorocyclopentane-1-carboxamide

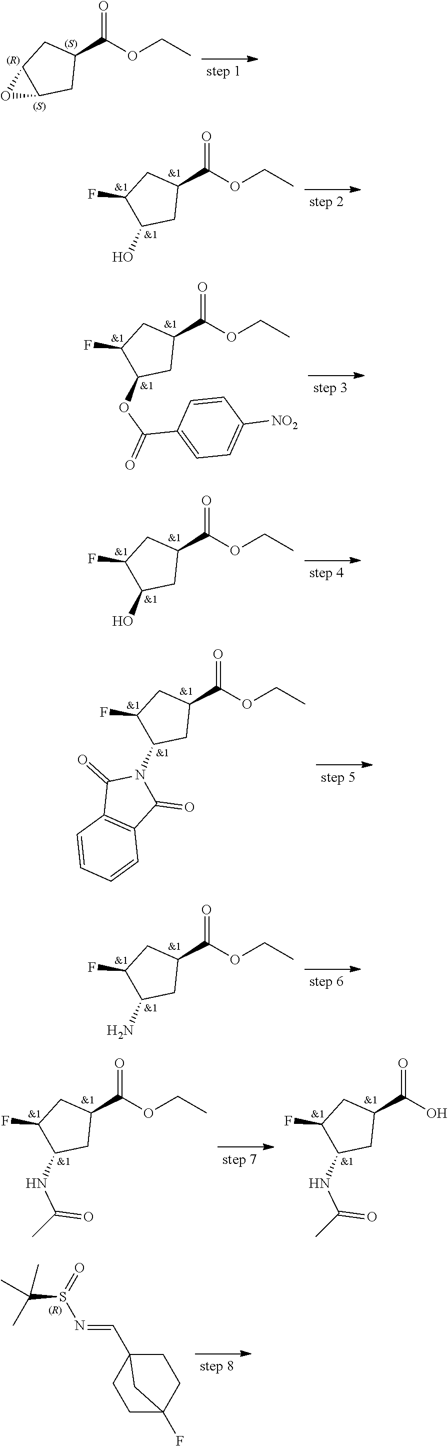

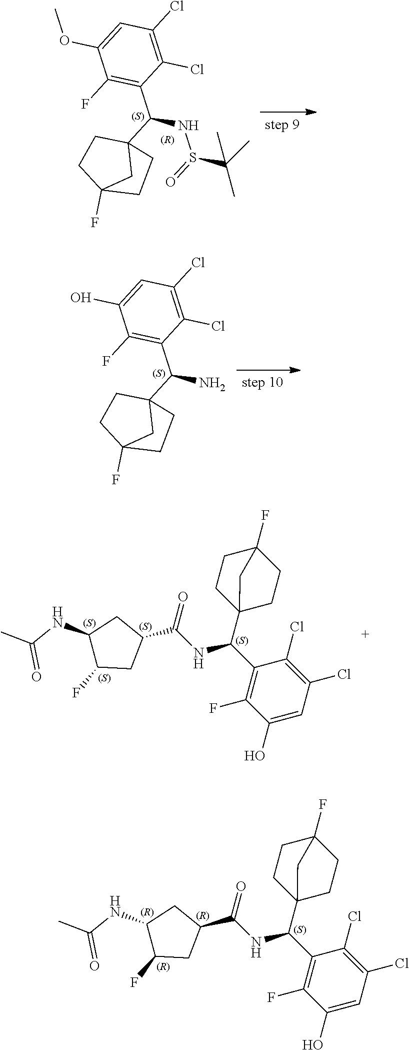

### Step 1. Synthesis of (f)-ethyl (1S,3S,4S)-3-fluoro-4-hydroxycyclopentane-1-carboxylate

A mixture of ethyl (1R,3s,5S)-6-oxabicyclo [3.1.0]hexane-3-carboxylate (9.5 g, 61 mmol) and Et₃N—(HF)₃ (20 g, 0.12 mol) was stirred for 5 h at 110° C. After cooling to room temperature the reaction was quenched by the addition of water (100 mL). The resulting mixture was extracted with ethyl acetate (3×100 mL). The combined organic layers were washed with water (1×100 mL) and dried over anhydrous Na₂SO₄. After filtration, the filtrate was concentrated under reduced pressure. The crude product was purified by silica gel chromatography (120 g column; eluting with petroleum ether/ethyl acetate; ratio: 10/1) to give (±)-ethyl (1S,3S,4S)-3-fluoro-4-hydroxycyclopentane-1-carboxylate (8.0 g, 0.05 mol) as a yellow oil. ¹H NMR (400 MHz, Chloroform-d) δ 4.85 (dddd, J=51.5, 5.6, 3.8, 1.6 Hz, 1H), 4.39 (ddt, J=10.8, 5.2, 2.4 Hz, 1H), 4.16 (q, J=7.1 Hz, 2H), 3.09 (dtd, J=10.0, 8.4, 6.1 Hz, 1H), 2.43 (dddd, J=29.4, 15.4, 10.0, 5.6 Hz, 1H), 2.34-2.08 (m, 2H), 1.97 (ddd, J=14.1, 8.4, 2.7 Hz, 1H), 1.27 (t, J=7.1 Hz, 3H).

### Step 2. Synthesis of (f)-(1R,2S,4S)-4-(ethoxycarbonyl)-2-fluorocyclopentyl 4-nitrobenzoate

To a mixture of (±)-ethyl (1S,3S,4S)-3-fluoro-4-hydroxycyclopentane-1-carboxylate (7.5 g, 43 mmol), 4-nitrobenzoic acid (8.5 g, 51 mmol) and triphenylphosphine (26 g, 98 mmol) was DIAD (20 g, 98 mmol) added dropwise at 0° C. under N₂. The solution was stirred for 12 h at 25° C. The reaction was quenched by the addition of water (100 mL) at room temperature. The resulting mixture was extracted with ethyl acetate (3×150 mL). The combined organic layers were washed with water (1×100 mL) and dried over anhydrous Na₂SO₄. After filtration, the filtrate was concentrated under reduced pressure. The crude product was purified by silica gel chromatography (120 g column; eluting with petroleum ether/ethyl acetate; ratio: 15/1) to give (±)-(1R,2S,4S)-4-(ethoxycarbonyl)-2-fluorocyclopentyl 4-nitrobenzoate (11.5 g, 35.4 mmol) as a yellow oil. ¹H NMR (400 MHz, DMSO-d6) δ 8.44-8.32 (m, 2H), 8.26-8.13 (m, 2H), 5.37-5.08 (m, 2H), 4.11 (q, J=7.1 Hz, 2H), 3.05 (dtd, J=10.3, 8.5, 6.0 Hz, 1H), 2.40 (tdd, J=22.3, 9.5, 5.7 Hz, 2H), 2.22 (dddd, J=23.6, 15.5, 6.9, 2.7 Hz, 2H), 1.22 (dt, J=14.3, 6.3 Hz, 3H).

### Step 3. Synthesis of (±)-ethyl (1S,3S,4R)-3-fluoro-4-hydroxycyclopentane-1-carboxylate

A mixture of (±)-(1R,2S,4S)-4-(ethoxycarbonyl)-2-fluorocyclopentyl 4-nitrobenzoate (9.5 g, 29 mmol) and lithium hydroxide (0.77 g, 32 mmol) in THF/EtOH/H₂O (30 ml, 4/1/1) was stirred for 2 hours at 25° C. The mixture was concentrated and the aqueous solution's pH was adjusted to 6. The reaction mixture was extracted with ethyl acetate (150 mL) three times. The organic layers were combined, dried over Na₂SO₄ and concentrated. The residue was purified by silica gel chromatography, eluting with petroleum ether/ethyl acetate 3/1 to give (±)-ethyl (1S,3S,4R)-3-fluoro-4-hydroxycyclopentane-1-carboxylate (4 g, 0.02 mol) as a colorless oil. ¹H NMR (400 MHz, Chloroform-d) δ 4.87 (dq, J=54.3, 3.9 Hz, 1H), 4.17 (q, J=7.1 Hz, 2H), 4.11-3.97 (m, 1H), 2.90-2.71 (m, 1H), 2.53 (s, 1H), 2.36 (dt, J=6.2, 3.1 Hz, 3H), 2.31-2.13 (m, 1H), 1.27 (t, J=7.0 Hz, 3H).

### Step 4. Synthesis of (±)-ethyl (1S,3S,4S)-3-(1,3-dioxoisoindolin-2-yl)-4-fluorocyclopentane-1-carboxylate

To a mixture of (±)-ethyl (1S,3S,4R)-3-fluoro-4-hydroxycyclopentane-1-carboxylate (5.7 g, 32 mmol), triphenylphosphane (10 g, 39 mmol) and isoindoline-1,3-dione (5.7 g, 39 mmol) in THF (100 mL) was DIAD (7.9 g, 39 mmol) added dropwise. The solution was stirred for 12 hours at 25° C. The reaction was quenched with water and extracted with ethyl acetate (200 mL*3). The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 10% to 80% B in 25 min; detector: UV 254 nm) to give (±)-ethyl (1S,3S,4S)-3-(1,3-dioxoisoindolin-2-yl)-4-fluorocyclopentane-1-carboxylate (3 g, 0.01 mol) as a white amorphous solid. ¹H NMR (400 MHz, DMSO-d₆) δ 7.98-7.75 (m, 4H), 5.45 (ddt, J=53.7, 6.6, 4.7 Hz, 1H), 4.70 (dtd, J=24.9, 8.6, 4.4 Hz, 1H), 4.11 (qd, J=7.1, 1.4 Hz, 2H), 3.23 (td, J=8.5, 4.2 Hz, 1H), 2.71-2.50 (m, 1H), 2.36 (ddd, J=14.1, 9.5, 4.8 Hz, 1H), 2.26-2.04 (m, 2H), 1.21 (t, J=7.1 Hz, 3H).

### Step 5. Synthesis of (f)-ethyl (1S,3S,4S)-3-amino-4-fluorocyclopentane-1-carboxylate

A mixture of (±)-ethyl (1S,3S,4S)-3-(1,3-dioxoisoindolin-2-yl)-4-fluorocyclopentane-1-carboxylate (500 mg, 1.64 mmol) and N₂H₄·H₂O (164 mg, 3.28 mmol) in EtOH (20 mL) was stirred for 2 hours at 70° C. The reaction mixture was filtered, the pad was washed with EtOH, and the filtrate was concentrated in vacuo to give (±)-ethyl (1S,3S,4S)-3-amino-4-fluorocyclopentane-1-carboxylate (235 mg, 1.1 mmol) as a yellow oil. LCMS RT 0.481 min, [M+H]⁺ 176, LCMS method B.

### Step 6. Synthesis of (f)-ethyl (1S,3S,4S)-3-acetamido-4-fluorocyclopentane-1-carboxylate

To a mixture of (±)-ethyl (1S,3S,4S)-3-amino-4-fluorocyclopentane-1-carboxylate (235 mg, 1.34 mmol) and triethylamine (407 mg, 4.02 mmol) in DCM (5 mL) was added acetyl chloride (158 mg, 2.01 mmol) dropwise. The solution was stirred for 2 hours at 0° C. The reaction was quenched with water (10 mL) and extracted with ethyl acetate (50 mL*3). The combined organic layers were washed with brine, dried over sodium sulfate, filtered and concentrated in vacuo to give (±)-ethyl (1S,3S,4S)-3-acetamido-4-fluorocyclopentane-1-carboxylate (200 mg, 921 μmol) as a yellow oil. LCMS RT 0.542 min, [M+H]⁺ 218, LCMS method C.

### Step 7. Synthesis of (±)-(1S,3S,4S)-3-acetamido-4-fluorocyclopentane-1-carboxylic acid

A mixture of (±)-ethyl (1S,3S,4S)-3-acetamido-4-fluorocyclopentane-1-carboxylate (240 mg, 1.10 mmol) and LiOH (79.4 mg, 3.31 mmol) was dissolved in MeOH/H₂O (4 ml, 3/1). The solution was stirred at 25° C. for 3 hours. The mixture was concentrated and the residue's pH was adjusted to 6. The solution was concentrated in vacuo to give (±)-(1S,3S,4S)-3-acetamido-4-fluorocyclopentane-1-carboxylic acid (200 mg, 1.06 mmol) as a white amorphous solid, which was used in the next step without purification. LCMS RT 0.278 min, [M+H]⁺ 190, LCMS method A.

### Step 8. Synthesis of (R)—N—((S)-(2,3-dichloro-6-fluoro-5-methoxyphenyl) (4-fluorobicyclo [2.2.1]heptan-1-yl) methyl)-2-methylpropane-2-sulfinamide

To a solution of 1,2-dichloro-4-fluoro-5-methoxybenzene (1 g, 6 mmol) in THF (80 mL) was added LDA (2 M in THF, 5.5 mL, 11 mmol) dropwise at −78° C. under a N₂ atmosphere. The reaction mixture was stirred at −78° C. for 1 hour prior to the addition of a solution of (R)—N-((4-fluorobicyclo[2.2.1]heptan-1-yl)methylene)-2-methylpropane-2-sulfinamide (1 g, 4 mmol) in THF (5 mL) at −78° C. under N₂. The mixture was stirred for 2 hours at −78° C. The reaction was quenched with saturated NH₄Cl solution (100 mL), and the mixture was extracted with EtOAc (3*100 mL). The combined organic extracts were washed with brine (100 mL) and dried over anhydrous Na₂SO₄. The resulting crude material was purified by flash chromatography (acetonitrile/water) to give (R)—N—((S)-(2,3-dichloro-6-fluoro-5-methoxyphenyl) (4-fluorobicyclo [2.2.1]heptan-1-yl) methyl)-2-methylpropane-2-sulfinamide (880 mg, 2.00 mmol) as a yellow oil. LCMS RT 1.10 min, [M+H]⁺ 440, LCMS method C.

### Step 9. Synthesis of (S)-3-(amino(4-fluorobicyclo[2.2.1]heptan-1-yl)methyl)-4,5-dichloro-2-fluorophenol

To (R)—N—((S)-(2,3-dichloro-6-fluoro-5-methoxyphenyl) (4-fluorobicyclo [2.2.1]heptan-1-yl) methyl)-2-methylpropane-2-sulfinamide (940 mg, 2.13 mmol) was added HBr (40 ml, 33% in AcOH). The solution was stirred for 24 hours at 100° C. The resulting mixture was concentrated under reduced pressure. The mixture was adjusted to pH 7 with NaOH (4 N, aq.). The resulting mixture was extracted with ethyl acetate (3×50 mL). The combined organic layers were washed with water (50 mL) and dried over anhydrous Na₂SO₄. After filtration, the filtrate was concentrated under reduced pressure. The resulting crude material was purified by flash chromatography (acetonitrile/water) to give (S)-3-(amino(4-fluorobicyclo[2.2.1]heptan-1-yl)methyl)-4,5-dichloro-2-fluorophenol (630 mg, 1.96 mmol) as a colorless oil. LCMS RT 0.66 min, [M+H]⁺ 322, LCMS method D.

### Step 10. Synthesis of (1S,3S,4S)-3-acetamido-N—((S)-(2,3-dichloro-6-fluoro-5-hydroxyphenyl)(4-fluorobicyclo[2.2.1]heptan-1-yl)methyl)-4-fluorocyclopentane-1-carboxamide and (1R,3R,4R)-3-acetamido-N—((S)-(2,3-dichloro-6-fluoro-5-hydroxyphenyl)(4-fluorobicyclo[2.2.1]heptan-1-yl)methyl)-4-fluorocyclopentane-1-carboxamide

To a mixture of (S)-3-(amino(4-fluorobicyclo[2.2.1]heptan-1-yl)methyl)-4,5-dichloro-2-fluorophenol (50 mg, 0.16 mmol), (±)-(1S,3S,4S)-3-acetamido-4-fluorocyclopentane-1-carboxylic acid (29 mg, 0.16 mmol) and NaHCO₃ (39 mg, 0.47 mmol) in DMF (1 mL) was added HATU (88 mg, 0.23 mmol). The mixture was stirred for 1 h at 25° C. The reaction mixture was diluted with water (50 mL), and the aqueous phase was extracted with ethyl acetate (50 ml) three times. The combined organic layers were washed with brine, dried over sodium sulfate, filtered and concentrated in vacuo. The residue was purified by reverse phase flash chromatography (column, C18 silica gel; mobile phase A: water, mobile phase B: acetonitrile; 0% to 100% gradient in 10 min; detector: UV 220 nm. The resulting crude material was purified by chiral preparative HPLC (column: Sunfire prep C18 column, 30*150 mm, 5 μm; mobile phase A: water (0.1% formic acid), mobile phase B: acetonitrile; flow rate: 60 mL/min; gradient: 40% B to 51% B in 7 min, then 51% B; wavelength: 254/220 nm; RT1 (min): 6.5) to give (±)-(1S,3S,4S)-3-acetamido-N—((S)-(2,3-dichloro-6-fluoro-5-hydroxyphenyl)(4-fluorobicyclo[2.2.1]heptan-1-yl)methyl)-4-fluorocyclopentane-1-carboxamide (40 mg, 81 μmol) as an off-white solid.

The product was further purified by chiral preparative HPLC (column: CHIRALPAK IE, 2*25 cm, 5 μm; mobile phase A: hexane (0.5% 2M NH₃-MeOH), mobile phase B: EtOH; flow rate: 20 mL/min; gradient: 20% B isocratic; wavelength: 220/254 nm; RT1 (min): 6.18; RT2 (min): 7.67; sample solvent: EtOH; injection volume: 0.35 mL) to give (1S,3S,4S)-3-acetamido-N—((S)-(2,3-dichloro-6-fluoro-5-hydroxyphenyl) (4-fluorobicyclo [2.2.1]heptan-1-yl) methyl)-4-fluorocyclopentane-1-carboxamide and (1R,3R,4R)-3-acetamido-N—((S)-(2,3-dichloro-6-fluoro-5-hydroxyphenyl) (4-fluorobicyclo [2.2.1]heptan-1-yl) methyl)-4-fluorocyclopentane-1-carboxamide, both as an off-white amorphous solid.

Isomer 1: 5.2 mg, 10 μmol. ¹H NMR (400 MHz, DMSO-d6) δ 8.14 (d, J=8.4 Hz, 1H), 7.93 (d, J=6.9 Hz, 1H), 7.08 (d, J=8.2 Hz, 1H), 5.51-5.35 (m, 1H), 4.83 (dq, J=53.3, 5.0 Hz, 1H), 4.07 (d, J=11.8 Hz, 1H), 3.00 (p, J=7.9 Hz, 1H), 2.34-2.15 (m, 1H), 1.99 (dt, J=14.0, 7.7 Hz, 1H), 1.80 (d, J=4.6 Hz, 6H), 1.75-1.61 (m, 5H), 1.60-1.35 (m, 4H). LCMS RT 0.958 min, [M+H]⁺ 493.15, LCMS method B.

Isomer 2: 5.7 mg, 11 μmol. ¹H NMR (400 MHz, DMSO-d6) δ 10.60 (s, 1H), 8.36-8.11 (m, 1H), 7.93 (d, J=6.7 Hz, 1H), 7.11 (d, J=8.2 Hz, 1H), 5.51-5.29 (m, 1H), 4.86 (dq, J=53.2, 4.6 Hz, 1H), 4.28-3.97 (m, 1H), 3.08-2.89 (m, 1H), 2.39-2.24 (m, 1H), 2.04-1.83 (m, 4H), 1.80 (s, 5H), 1.74-1.60 (m, 4H), 1.54 (dq, J=21.6, 10.1, 9.0 Hz, 3H). LCMS RT 1.522 min, [M+H]⁺ 493.10, LCMS method B.

### Example 26

### (2r,4S)—N—((S)-(3-chloro-2,6-difluorophenyl)(cyclopentyl)methyl)-7-methyl-6-oxo-5,7-diazaspiro[3.5]nonane-2-carboxamide and (2s,4R)—N—((S)-(3-chloro-2,6-difluorophenyl)(cyclopentyl)methyl)-7-methyl-6-oxo-5,7-diazaspiro[3.5]nonane-2-carboxamide

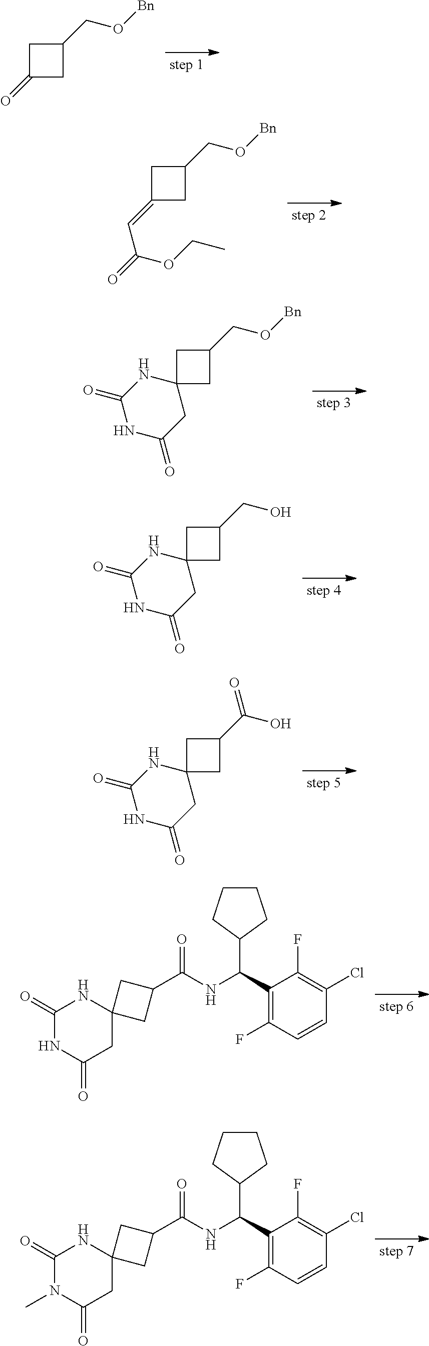

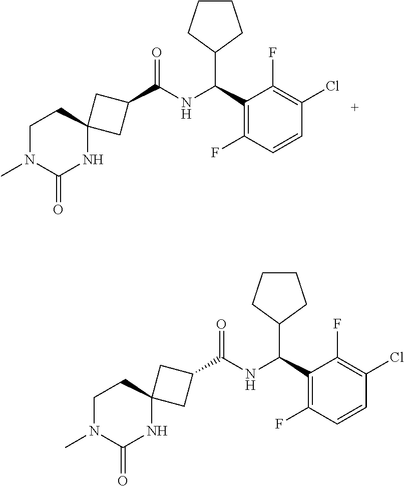 [SMILES: CN1CC[C@]2(C[C@@H](C(=O)N[C@H](c3c(F)ccc(Cl)c3F)C3CCCC3)C2)NC1=O ; CN1CC[C@]2(C[C@H](C(=O)N[C@H](c3c(F)ccc(Cl)c3F)C3CCCC3)C2)NC1=O]

### Step 1. Synthesis of ethyl 2-(3-((benzyloxy)methyl)cyclobutylidene)acetate

To a mixture of 3-((benzyloxy)methyl) cyclobutan-1-one (10 g, 53 mmol) and ethyl 2-(diethoxyphosphoryl) acetate (14 g, 63 mmol) in THF (100 mL) was added NaH (1.3 g, 53 mmol) in portions at 0° C. The mixture was stirred for 1 hour at room temperature. The reaction was quenched with saturated NH₄Cl (aq.) (30 ml) and the aqueous phase was extracted with ethyl acetate (100 mL) three times. The combined organic layers were washed with brine, dried over sodium sulfate, filtered and concentrated in vacuo. The resulting crude material was purified by silica gel chromatography (100 g column; eluting with petroleum ether:ethyl acetate 5:1) to give ethyl 2-(3-((benzyloxy)methyl)cyclobutylidene)acetate (13.46 g, 51.70 mmol) as a colorless oil. LCMS RT 1.082 min, [M+H]⁺ 261, LCMS method C.

### Step 2. Synthesis of 2-((benzyloxy)methyl)-5,7-diazaspiro[3.5]nonane-6,8-dione

To a mixture of ethyl 2-(3-((benzyloxy)methyl)cyclobutylidene)acetate (11 g, 42 mmol) and urea (15 g, 0.2 mol) in NMP (120 mL) was added DBU (25 mL, 0.17 mol) at room temperature. The mixture was stirred for 16 h at 140° C. After cooling to room temperature, the reaction mixture was diluted with water (150 mL), and the aqueous phase was extracted with ethyl acetate (150 mL) three times. The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 10% to 60% B in 10 min; detector: UV 220 nm) to give 2-((benzyloxy)methyl)-5,7-diazaspiro[3.5]nonane-6,8-dione (7.07 g, 25.8 mmol) as a yellow oil. LCMS RT 0.902 min, [M+H]⁺ 275, LCMS method C.

### Step 3. Synthesis of 2-(hydroxymethyl)-5,7-diazaspiro[3.5]nonane-6,8-dione

A mixture of 2-((benzyloxy)methyl)-5,7-diazaspiro[3.5]nonane-6,8-dione (5.5 g, 20 mmol) and Pd/C (0.21 g) in MeOH (60 mL) was treated with H₂ (20 atm) and stirred at room temperature overnight. The reaction mixture was filtered through a pad of Celite, the pad was washed with MeOH (200 mL), and the filtrate was concentrated in vacuo. The resulting crude material was purified by silica gel chromatography (100 g column; eluting with DCM:MeOH 25:1) to give 2-(hydroxymethyl)-5,7-diazaspiro[3.5]nonane-6,8-dione (3.22 g, 17.5 mmol) as a white solid. LCMS RT 0.202 min, [M+H]⁺ 185, LCMS method C.

### Step 4. Synthesis of 6,8-dioxo-5,7-diazaspiro[3.5]nonane-2-carboxylic acid

To a mixture of 2-(hydroxymethyl)-5,7-diazaspiro[3.5]nonane-6,8-dione (400 mg, 2.17 mmol) in H₂O (5 mL) was added a solution of KMnO₄ (343 mg, 2.17 mmol) in H₂O (5 mL) at 0° C. The mixture was stirred for 3 hours at room temperature. The reaction mixture was filtered through a pad of Celite, the pad was washed with MeOH (50 mL), and the filtrate was concentrated in vacuo to afford 6,8-dioxo-5,7-diazaspiro[3.5]nonane-2-carboxylic acid (400 mg, 2.02 mmol) as a brown solid. LCMS RT 0.119 min, [M+H]⁺ 199, LCMS method D.

### Step 5. Synthesis of (S)—N-((3-chloro-2,6-difluorophenyl)(cyclopentyl)methyl)-6,8-dioxo-5,7-diazaspiro[3.5]nonane-2-carboxamide

To a mixture of 6,8-dioxo-5,7-diazaspiro [3.5]nonane-2-carboxylic acid (400 mg, 2.02 mmol), (S)-(3-chloro-2,6-difluorophenyl) (cyclopentyl)methanamine (496 mg, 2.02 mmol) and TEA (612 mg, 6.0m mol) in DMF (4 mL) was added T₃P (1.93 g, 6.06 mmol) at room temperature. The mixture was stirred for 1 h at room temperature. The reaction mixture was diluted with water (20 mL), and the aqueous phase was extracted with ethyl acetate (20 mL) three times. The combined organic layers were washed with brine, dried over sodium sulfate, filtered and concentrated in vacuo. The resulting crude material was purified by preparative HPLC (column: XB ridge Prep OBD C18 Column, 30*150 mm, 5 μm; mobile phase A: water (10 mM NH₄HCO₃), mobile phase B: acetonitrile; flow rate: 60 mL/min; gradient: 25% B to 55% B in 8 min, then 55% B; wavelength: 220 nm; RT1 (min): 7.68) to give (S)—N-((3-chloro-2,6-difluorophenyl)(cyclopentyl)methyl)-6,8-dioxo-5,7-diazaspiro[3.5]nonane-2-carboxamide [3.5]nonane-2-carboxamide (380 mg, 892 μmol) as a white amorphous solid. LCMS RT 1.060 min, [M+H]⁺ 390, LCMS method C.

### Step 6. Synthesis of (S)—N-((3-chloro-2,6-difluorophenyl)(cyclopentyl)methyl)-7-methyl-6,8-dioxo-5,7-diazaspiro[3.5]nonane-2-carboxamide

To a mixture of (S)—N-((3-chloro-2,6-difluorophenyl) (cyclopentyl)methyl)-6,8-dioxo-5,7-diazaspiro [3.5]nonane-2-carboxamide (140 mg, 329 μmol) in toluene (2 mL) was added 1,1-dimethoxy-N,N-dimethylethan-1-amine (131 mg, 986 μmol). The mixture was stirred for 2 h at 110° C. The mixture was concentrated in vacuo. The residue was purified by reverse phase flash chromatography (column: C18 silica gel; mobile phase A: water, mobile phase B: acetonitrile; gradient: 45% to 65% B in 15 min; detector: UV 220 nm) to give (S)—N-((3-chloro-2,6-difluorophenyl)(cyclopentyl)methyl)-7-methyl-6,8-dioxo-5,7-diazaspiro[3.5]nonane-2-carboxamide (100 mg, 227 μmol) as a colorless oil. LCMS RT 1.135 min, [M+H]⁺ 440, LCMS method C.

### Step 7. Synthesis of (2r,4S)—N—((S)-(3-chloro-2,6-difluorophenyl)(cyclopentyl)methyl)-7-methyl-6-oxo-5,7-diazaspiro[3.5]nonane-2-carboxamide and (2s,4R)—N—((S)-(3-chloro-2,6-difluorophenyl)(cyclopentyl)methyl)-7-methyl-6-oxo-5,7-diazaspiro[3.5]nonane-2-carboxamide

To a mixture of (S)—N-((3-chloro-2,6-difluorophenyl) (cyclopentyl)methyl)-7-methyl-6,8-dioxo-5,7-diazaspiro[3.5]nonane-2-carboxamide (80 mg, 0.18 mmol) in THF (2 mL) were added BF₃-Et₂O (31 mg, 0.22 mmol) and NaBH₄ (6.9 mg, 0.18m mol) at 0° C. The mixture was stirred for 1 h at room temperature. The reaction mixture was diluted with water (10 mL), and the aqueous phase was extracted with ethyl acetate (10 mL) three times. The combined organic layers were washed with brine, dried over sodium sulfate, filtered and concentrated in vacuo. The resulting crude material was purified by preparative HPLC (column: YMC-Actus Triart C18 Ex RS, 30*150 mm, 5 μm; mobile phase A: water (10 mM NH₄HCO₃+0.1% NH₄OH), mobile phase B: acetonitrile; flow rate: 60 mL/min; gradient: 30% B to 55% B in 9 min, 55% B to 60% B in 9.5 min, then 60% B; wavelength: 220 nm; RT1 (min): 9.13) to give (S)—N-((3-chloro-2,6-difluorophenyl) (cyclopentyl) methyl)-7-methyl-6-oxo-5,7-diazaspiro [3.5]nonane-2-carboxamide (35 mg, 82 μmol) as an off-white solid. LCMS RT 1.503 min, [M+H]⁺ 426, LCMS method D.

The product was purified by preparative chiral HPLC (column: DZ-CHIRALPAK IC-3, 4.6*50 mm, 3.0 μm; mobile phase A: hexane:EtOH 70:30; flow rate: 1 mL/min; gradient: 0% B isocratic; injection volume: 0.5 mL). Lyophilization yielded (2r,4S)—N—((S)-(3-chloro-2,6-difluorophenyl)(cyclopentyl)methyl)-7-methyl-6-oxo-5,7-diazaspiro[3.5]nonane-2-carboxamide and (2s,4R)—N—((S)-(3-chloro-2,6-difluorophenyl)(cyclopentyl)methyl)-7-methyl-6-oxo-5,7-diazaspiro[3.5]nonane-2-carboxamide, both as an off-white amorphous solid.

Isomer 1: 2.3 mg, 5.4 μmol. ¹H NMR (400 MHz, DMSO-d₆) δ 8.37 (d, J=7.4 Hz, 1H), 7.53 (td, J=8.7, 5.5 Hz, 1H), 7.12 (t, J=9.6 Hz, 1H), 6.70 (s, 1H), 4.82 (dd, J=11.1, 7.4 Hz, 1H), 3.04 (t, J=6.0 Hz, 2H), 2.94 (dq, J=10.0, 4.9 Hz, 1H), 2.71 (s, 3H), 2.41 (d, J=9.1 Hz, 1H), 2.25 (t, J=11.0 Hz, 1H), 2.15 (q, J=14.3, 12.8 Hz, 2H), 2.03 (d, J=12.6 Hz, 1H), 1.90 (d, J=8.2 Hz, 1H), 1.74 (dd, J=7.4, 4.7 Hz, 2H), 1.59 (s, 3H), 1.51 (dt, J=16.6, 9.3 Hz, 1H), 1.38-1.30 (m, 1H), 1.24 (s, 1H), 1.00 (s, 1H). LCMS RT 1.537 min, [M+H]⁺ 426, LCMS method D;

Isomer 2: 3.1 mg, 7.3 μmol. ¹H NMR (400 MHz, DMSO-d₆) δ 8.27 (d, J=7.5 Hz, 1H), 7.53 (td, J=8.7, 5.5 Hz, 1H), 7.12 (t, J=9.3 Hz, 1H), 6.54 (s, 1H), 4.83 (dd, J=11.2, 7.5 Hz, 1H), 3.14 (t, J=5.9 Hz, 2H), 2.72 (s, 3H), 2.41 (d, J=9.0 Hz, 1H), 2.26 (t, J=11.0 Hz, 1H), 2.16 (t, J=10.1 Hz, 1H), 2.11-2.01 (m, 2H), 2.00 (s, 1H), 1.94-1.85 (m, 1H), 1.80 (t, J=5.9 Hz, 2H), 1.64-1.51 (m, 4H), 1.49 (dd, J=15.5, 7.5 Hz, 1H), 1.38-1.30 (m, 1H), 1.00 (s, 1H). LCMS RT 1.537 min, [M+H]⁺ 426, LCMS method D.

### Example 27

### (1R,3S,4R)-3-acetamido-4-amino-N—((S)-(2,3-dichloro-6-fluorophenyl)(1-methylcyclopentyl)methyl)cyclopentane-1-carboxamide and (1S,3S,4R)-3-acetamido-4-amino-N—((S)-(2,3-dichloro-6-fluorophenyl)(1-methylcyclopentyl)methyl)cyclopentane-1-carboxamide

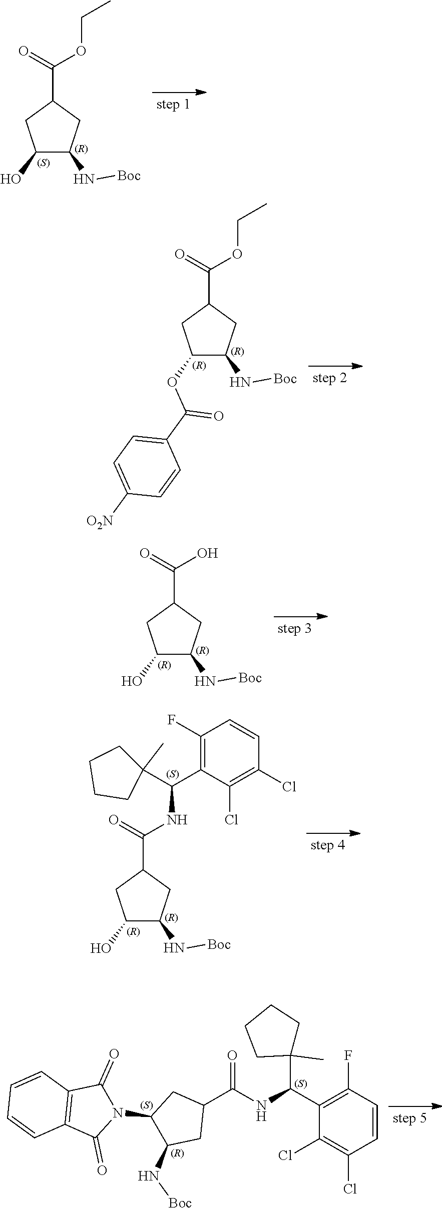

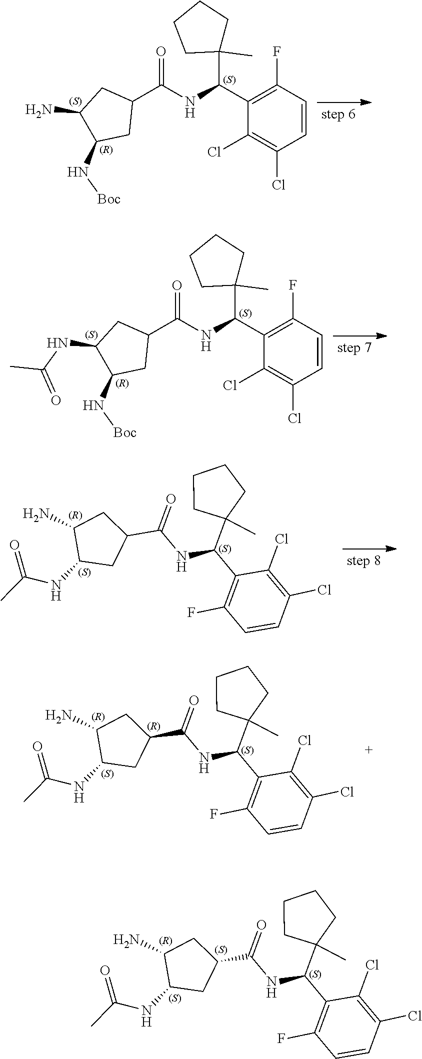

### Step 1. Synthesis of (1R,2R)-2-((tert-butoxycarbonyl)amino)-4-(ethoxycarbonyl)cyclopentyl 4-nitrobenzoate

To a stirred solution of ethyl (3R,4S)-3-((tert-butoxycarbonyl)amino)-4-hydroxycyclopentane-1-carboxylate (1.91 g, 7 mmol), 4-nitrobenzoic acid (1.17 g, 7 mmol) and triphenylphosphine (1.83 g, 7 mmol) in THF (20 mL) was added DIAD (1.41 g, 7 mmol) dropwise a t 0° C. The resulting mixture was stirred for 2 h at 25° C. The reaction mixture was diluted with water (30 mL), and the aqueous phase was extracted with ethyl acetate (30 mL) three times. The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 10% to 100% B in 30 min; detector: UV 254 nm) to afford (1R,2R)-2-((tert-butoxycarbonyl)amino)-4-(ethoxycarbonyl) cyclopentyl 4-nitrobenzoate (1.2 g, 2.8 mmol) as a white solid. L CMS RT=1.23 min, [M+H]⁺ 423, LCMS method A.

### Step 2. Synthesis of (3R,4R)-3-((tert-butoxycarbonyl)amino)-4-hydroxycyclopentane-1-carboxylic acid

To a solution of (1R,2R)-2-((tert-butoxycarbonyl) amino)-4-(ethoxycarbonyl) cyclo pentyl 4-nitrobenzoate (500 mg, 1.18 mmol) in MeOH (4 mL) was added lithium hydroxide (142 mg, 5.92 mmol) in H₂O (1 mL). The mixture was stirred at 25° C. for 1 hour. The solution was concentrated under reduced pressure to remove MeOH. The residue was acidified to pH 5-6 with HCl (2N). The solution was concentrated to dryness under reduced pressure to give (3R,4R)-3-((tert-butoxycarbonyl) amino)-4-hydroxycyclopentane-1-carboxylic acid (270 mg, 1.10 mmol) as a white amorphous solid. LCMS RT 0.388 min, [M−H]⁻ 244, LCMS method B.

### Step 3. Synthesis of tert-butyl ((1R,2R)-4-(((S)-(2,3-dichloro-6-fluorophenyl) (1-methylcyclopentyl) methyl) carbamoyl)-2-hydroxycyclopentyl) carbamate

To a mixture of (3R,4R)-3-((tert-butoxycarbonyl) amino)-4-hydroxycyclopentane-1-carboxylic acid (270 mg, 1.10 mmol), (S)-(2,3-dichloro-6-fluorophenyl) (1-methylcyclopent yl) methanamine (365 mg, 1.32 mmol) and NaHCO₃ (370 mg, 4.40 mmol) in DMF (5 mL) was added HATU (837 mg, 2.20 mmol). The mixture was stirred at room temperature for 1 hour. The reaction was quenched with water (10 ml) and extracted with ethyl acetate (20 ml*3). The combined organic layers were washed with brine, dried over Na₂SO₄ and concentrated. The residue was purified by reverse phase flash chromatography (column: C18 silica gel; mobile phase A: water, mobile phase B: acetonitrile; gradient: 10% to 50% B in 10 min; detector: UV 254 nm) to give tert-butyl ((1R,2R)-4-(((S)-(2,3-dichloro-6-fluorophenyl) (1-methylcyclopentyl)methyl)carbamoyl)-2-hydroxycyclopentyl) carbamate (300 mg, 596 μmol) as a yellow oil. LCMS RT 1.129 min, m/z [M-56+H]+446, LCMS method C.

### Step 4. Synthesis of tert-butyl ((1R,2S)-4-(((S)-(2,3-dichloro-6-fluorophenyl)(1-methylcyclopentyl)methyl)carbamoyl)-2-(1,3-dioxoisoindolin-2-yl)cyclopentyl)carbamate

To a mixture of tert-butyl ((1R,2R)-4-(((S)-(2,3-dichloro-6-fluorophenyl) (1-methylcyclopentyl)methyl)carbamoyl)-2-hydroxycyclopentyl) carbamate (105 mg, 715 μmol) and triphenylphosphine (234 mg, 894 μmol) in THF (6 mL) was added DIAD (174 μL, 894 μmol) dropwise at 0° C. under a nitrogen atmosphere. The mixture was stirred for 16 hours at 25° C. The reaction mixture was diluted with water (10 mL), and the aqueous phase was extracted with ethyl acetate (40 mL) three times. The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 0% to 100% B in 20 min; detector: UV 254 nm) to give tert-butyl ((1R,2S)-4-(((S)-(2,3-dichloro-6-fluorophenyl) (1-methylcyclopentyl) methyl) carbamoyl)-2-(1,3-dioxoisoindolin-2-yl) cyclopentyl) carbamate (240 mg, 379 μmol) as a yellow oil. LCMS RT 1.279 min, [M-56+H]+576, LCMS method C.

### Step 5. Synthesis of tert-butyl ((1R,2S)-2-amino-4-(((S)-(2,3-dichloro-6-fluorophenyl)(1-methylcyclopentyl)methyl)carbamoyl)cyclopentyl)carbamate

To a solution of tert-butyl ((1R,2S)-4-(((S)-(2,3-dichloro-6-fluorophenyl) (1-methyl cyclopentyl) methyl) carbamoyl)-2-(1,3-dioxoisoindolin-2-yl) cyclopentyl) carbamate (220 mg, 348 μmol) in EtOH (4 mL) was added hydrazine hydrate (34.8 mg, 696 μmol). The mixture was heated at 70° C. for 2 hours. The reaction mixture was filtered, the collected solid was washed with EtOH, and the filtrate was concentrated in vacuo. The residue was purified by reverse phase flash chromatography (column: C18 silica gel; mobile phase A: water, mobile phase B: acetonitrile; gradient: 10% to 50% B in 10 min; detector: UV 254 nm) to give tert-butyl ((1R,2S)-2-amino-4-(((S)-(2,3-dichloro-6-fluorophenyl)(1-methylcyclopentyl)methyl)carbamoyl)cyclopentyl)carbamate (130 mg, 259 μmol) as a white amorphous solid. LCMS RT 1.035 min, [M+H]⁺ 502, LCMS method C.

### Step 6. Synthesis of tert-butyl ((1R,2S)-2-acetamido-4-(((S)-(2,3-dichloro-6-fluorophenyl)(1-methylcyclopentyl)methyl)carbamoyl)cyclopentyl)carbamate

To a mixture of tert-butyl ((1R,2S)-2-amino-4-(((S)-(2,3-dichloro-6-fluorophenyl)(1-methylcyclopentyl)methyl)carbamoyl)cyclopentyl)carbamate (120 mg, 239 μmol), acetic acid (17.2 mg, 287 μmol) and NaHCO₃ (80.2 mg, 955 μmol) in DMF (4 mL) was added HATU (182 mg, 478 μmol). The mixture was stirred at room temperature for 1 hour. The reaction was quenched with water (10 ml) and extracted with ethyl acetate (20 mL*3). The combined organic layers were washed with brine, dried over Na₂SO₄ and concentrated. The residue w as purified by reverse phase flash chromatography (column: C18 silica gel; mobile phase A: water, mobile phase B: acetonitrile; gradient: 10% to 50% B in 10 min; detector: UV 254 nm) to give tert-butyl ((1R,2S)-2-acetamido-4-(((S)-(2,3-dichloro-6-fluorophenyl)(1-methylcyclopentyl)methyl)carbamoyl)cyclopentyl)carbamate (100 mg, 184 μmol) as a yellow oil. LCMS RT 1.238 min, [M-100+H]+444, LCMS method B.

### Step 7. Synthesis of (1R,3S,4R)-3-acetamido-4-amino-N—((S)-(2,3-dichloro-6-fluorophenyl)(1-methylcyclopentyl)methyl)cyclopentane-1-carboxamide and (1S,3S,4R)-3-acetamido-4-amino-N—((S)-(2,3-dichloro-6-fluorophenyl)(1-methylcyclopentyl)methyl)cyclopentane-1-carboxamide

A solution of tert-butyl ((1R,2S)-2-acetamido-4-(((S)-(2,3-dichloro-6-fluorophenyl)(1-methylcyclopentyl)methyl)carbamoyl)cyclopentyl)carbamate (100 mg, 184 μmol) in HCl (4 mL, 4 N in MeOH) was stirred at 25° C. for 1 hour. The solution was concentrated under reduced pressure. The residue was purified by reverse phase flash chromatography (column: C18 silica gel; mobile phase A: water, mobile phase B: acetonitrile; gradient: 10% to 50% B in 10 min; detector: UV 254 nm) to give (3S,4R)-3-acetamido-4-amino-N—((S)-(2,3-dichloro-6-fluorophenyl)(1-methylcyclopentyl)methyl)cyclopentane-1-carboxamide (50 mg, 0.11 mmol) as a white amorphous solid. LCMS RT 0.927 min, [M+H]⁺ 444, LCMS method C.

### Step 8. Synthesis of (1R,3S,4R)-3-acetamido-4-amino-N—((S)-(2,3-dichloro-6-fluorophenyl)(1-methylcyclopentyl)methyl)cyclopentane-1-carboxamide and (1S,3S,4R)-3-acetamido-4-amino-N—((S)-(2,3-dichloro-6-fluorophenyl)(1-methylcyclopentyl)methyl)cyclopentane-1-carboxamide

(3S,4R)-3-acetamido-4-amino-N—((S)-(2,3-dichloro-6-fluorophenyl)(1-methylcyclopentyl)methyl)cyclopentane-1-carboxamide (50 mg, 0.11 mmol) was purified by chiral preparative HPLC (column: CHIRALPAK IH, 2*25 cm, 5 μm; mobile phase A: hexane (0.5% 2 M NH₃ in MeOH), mobile phase B: EtOH:DCM 1:1; flow rate: 20 mL/min; gradient: 15% B isocratic; wavelength: 220/254 nm; RT1 (min): 6.23; RT2 (min): 7.94; sample solvent: EtOH:DCM 1:1; injection volume: 0.25 mL) to give (1R,3S,4R)-3-acetamido-4-amino-N—((S)-(2,3-dichloro-6-fluorophenyl)(1-methylcyclopentyl)methyl)cyclopentane-1-carboxamide and (1S,3S,4R)-3-acetamido-4-amino-N—((S)-(2,3-dichloro-6-fluorophenyl)(1-methylcyclopentyl)methyl)cyclopentane-1-carboxamide, both as a white amorphous solid.

Isomer 1: 5.7 mg, 13 μmol. ¹H NMR (400 MHz, DMSO-d₆) δ 8.08 (d, J=8.9 Hz, 1H), 7.59 (s, 2H), 7.25 (t, J=9.9 Hz, 1H), 5.47 (d, J=8.5 Hz, 1H), 3.90 (s, 1H), 3.31 (s, 1H), 3.12-3.04 (m, 2H), 2.31 (s, 1H), 1.82 (d, J=6.8 Hz, 3H), 1.59 (s, 9H), 1.37 (s, 1H), 1.23 (s, 3H), 0.96 (s, 3H). LCMS RT 1.298 min, [M+H]⁺ 444.15, LCMS method C

Isomer 2: 4.8 mg, 11 μmol. ¹H NMR (400 MHz, DMSO-d₆) δ 8.10 (s, 1H), 7.61 (dd, J=9.0, 4.9 Hz, 2H), 7.25 (t, J=9.9 Hz, 1H), 5.46 (d, J=8.4 Hz, 1H), 3.89 (s, 1H), 1.82 (s, 3H), 1.70 (d, J=9.6 Hz, 4H), 1.59 (s, 9H), 1.36 (d, J=10.6 Hz, 1H), 1.25 (d, J=11.8 Hz, 2H), 0.99-0.93 (m, 3H). LCMS RT 0.938 min, [M+H]⁺ 444, LCMS method D.

### Example 28

### (1R,3S,4R)-3-acetamido-N—((S)-(2,3-dichloro-6-fluorophenyl)(4-fluorobicyclo[2.2.1]heptan-1-yl)methyl)-4-((2,2,2-trifluoroethyl)amino)cyclopentane-1-carboxamide and (1S,3S,4R)-3-acetamido-N—((S)-(2,3-dichloro-6-fluorophenyl)(4-fluorobicyclo[2.2.1]heptan-1-yl)methyl)-4-((2,2,2-trifluoroethyl)amino)cyclopentane-1-carboxamide

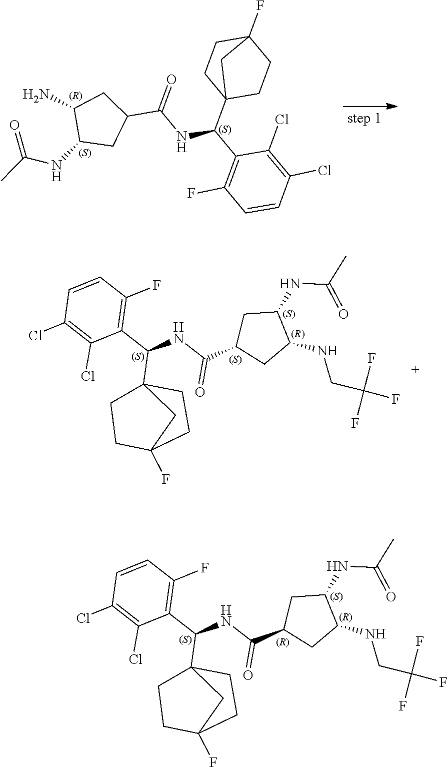

### Step 1. Synthesis of (1R,3S,4R)-3-acetamido-N—((S)-(2,3-dichloro-6-fluorophenyl)(4-fluorobicyclo[2.2.1]heptan-1-yl)methyl)-4-((2,2,2-trifluoroethyl)amino)cyclopentane-1-carboxamide and (1S,3S,4R)-3-acetamido-N—((S)-(2,3-dichloro-6-fluorophenyl)(4-fluorobicyclo[2.2.1]heptan-1-yl)methyl)-4-((2,2,2-trifluoroethyl)amino)cyclopentane-1-carboxamide

To a mixture of (3S,4R)-3-acetamido-4-amino-N—((S)-(2,3-dichloro-6-fluorophenyl)(4-fluorobicyclo[2.2.1]heptan-1-yl)methyl)cyclopentane-1-carboxamide (70 mg, 0.15 mmol) and 4 Å molecular sieves (200 mg) in MeOH (4 mL) was added 2,2,2-trifluoroacetaldehyde (22 mg, 0.22 mmol). The mixture was stirred at 25° C. for 30 min prior to the addition of NaBH₃CN (28 mg, 0.44 mmol). The mixture was stirred for 16 hours at 25° C. The reaction mixture was diluted with water (5 mL), and the aqueous phase was extracted with ethyl acetate (10 mL) three times. The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 10% to 50% B in 10 min; detector: UV 254 nm) to give a yellow oil, which w as further purified by chiral preparative HPLC (column: (R, R)-WHELK-01-Kromasil, 2.11*25 cm, 5 μm; mobile phase A: hexane (0.5% 2M NH₃ in MeOH), mobile phase B: isopropanol:DCM 1:1; flow rate: 20 mL/min; gradient: 40% B isocratic; wavelength: 220/254 nm RT1 (min): 14.62; RT2 (min): 22.08; sample solvent: EtOH:DCM 1:1; injection volume: 0.7 mL) to give (1S,3S,4R)-3-acetamido-N—((S)-(2,3-dichloro-6-fluorophenyl)(4-fluorobicyclo[2.2.1]heptan-1-yl)methyl)-4-((2,2,2-trifluoroethyl)amino)cyclopentane-1-carboxamide and (1R,3S,4R)-3-acetamido-N—((S)-(2,3-dichloro-6-fluorophenyl)(4-fluorobicyclo[2.2.1]heptan-1-yl)methyl)-4-((2,2,2-trifluoroethyl)amino)cyclopentane-1-carboxamide, both as a white amorphous solid.

Isomer 1:10 mg, 0.022 mmol. LCMS RT 1.078 min, [M+H]⁺ 556, LCMS method D. ¹H NMR (400 MHz, DMSO-d₆) δ 8.13 (d, J=8.2 Hz, 1H), 7.61 (dd, J=8.9, 5.0 Hz, 1H), 7.48 (d, J=7.4 Hz, 1H), 7.25 (dd, J=10.7, 9.0 Hz, 1H), 5.48 (d, J=7.9 Hz, 1H), 4.12-3.84 (m, 1H), 3.28-3.11 (m, 3H), 2.96 (d, J=6.3 Hz, 1H), 2.11 (q, J=7.4 Hz, 1H), 1.95-1.43 (m, 16H).

Isomer 2: 7 mg, 0.016 mmol. LCMS RT 1.078 min, [M+H]⁺ 556, LCMS method D. ¹H NMR (400 MHz, DMSO-d₆) δ 8.14 (d, J=8.2 Hz, 1H), 7.62 (dd, J=9.0, 5.1 Hz, 1H), 7.49 (d, J=7.5 Hz, 1H), 7.26 (dd, J=10.6, 9.0 Hz, 1H), 5.50 (d, J=8.1 Hz, 1H), 4.06 (p, J=6.0 Hz, 1H), 3.24-3.04 (m, 1H), 3.00-2.92 (m, 3H), 2.09 (q, J=7.3 Hz, 1H), 1.84 (s, 4H), 1.91-1.75 (m, 4H), 1.68 (qd, J=18.8, 17.1, 7.3 Hz, 5H), 1.57 (d, J=8.1 Hz, 2H), 0.06 (s, 2H)

### Example 29

### (1R,3S,4R)-3-acetamido-N—((S)-(2,3-dichloro-6-fluorophenyl)(4-fluorobicyclo[2.2.1]heptan-1-yl)methyl)-4-(methylamino)cyclopentane-1-carboxamide and (1S,3S,4R)-3-acetamido-N—((S)-(2,3-dichloro-6-fluorophenyl)(4-fluorobicyclo[2.2.1]heptan-1-yl)methyl)-4-(methylamino)cyclopentane-1-carboxamide

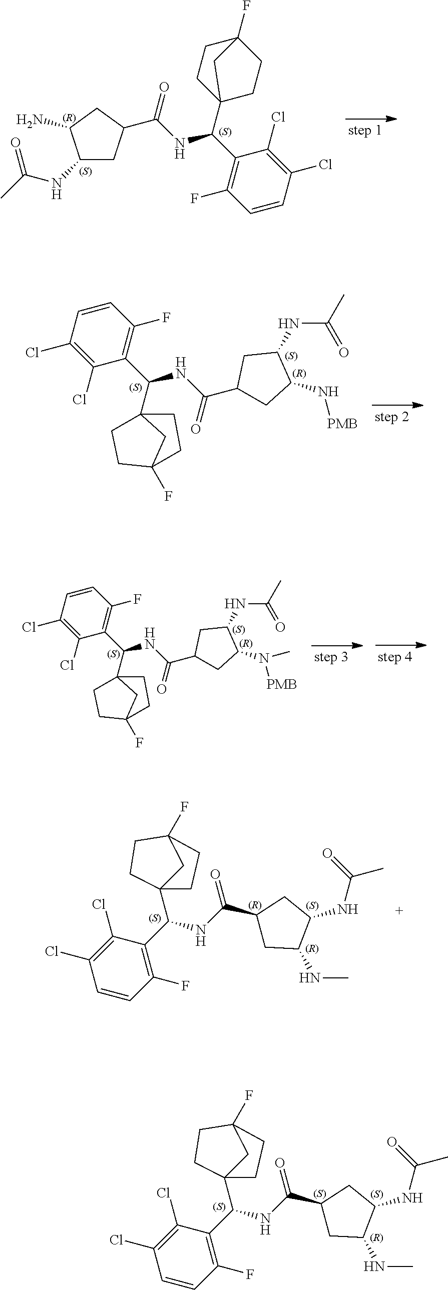

### Step 1. Synthesis of (3S,4R)-3-acetamido-N—((S)-(2,3-dichloro-6-fluorophenyl)(4-fluorobicyclo[2.2.1]heptan-1-yl)methyl)-4-((4-methoxybenzyl)amino)cyclopentane-1-carboxamide

To a mixture of (3S,4R)-3-acetamido-4-amino-N—((S)-(2,3-dichloro-6-fluorophenyl)(4-fluorobicyclo[2.2.1]heptan-1-yl)methyl)cyclopentane-1-carboxamide (70 mg, 0.15 mmol) in MeOH (5 mL) was added 4-methoxybenzaldehyde (30 mg, 0.22 mmol). The mixture was stirred at room temperature for 2 hours prior to the addition of NaBH₃CN (28 mg, 0.44 mmol) in portions at 0° C. under a nitrogen atmosphere. The mixture was stirred for 16 hours at room temperature. The mixture was diluted with water (5 mL), and the aqueous phase was extracted with ethyl acetate (10 mL*3). The combined organic layers were washed with brine, dried over sodium sulfate, filtered and concentrated in vacuo. The residue was purified by re verse phase flash chromatography (column: C18 silica gel; mobile phase A: water, mobile phase B: acetonitrile; gradient: 10% to 50% B in 10 min; detector: UV 254 nm) to give (3S,4R)-3-acetamido-N—((S)-(2,3-dichloro-6-fluorophenyl) (4-fluorobicyclo[2.2.1]heptan-1-yl)methyl)-4-((4-methoxybenzyl)amino)cyclopentane-1-carboxamide (50 mg, 84 μmol) as a yellow oil. LCMS RT 0.847 min, [M+H]⁺ 594, LCMS method C.

### Step 2. Synthesis of (3S,4R)-3-acetamido-N—((S)-(2,3-dichloro-6-fluorophenyl)(4-fluorobicyclo[2.2.1]heptan-1-yl)methyl)-4-((4-methoxybenzyl)(methyl)amino)cyclopentane-1-carboxamide

To a mixture of (3S,4R)-3-acetamido-N—((S)-(2,3-dichloro-6-fluorophenyl)(4-fluoro bicyclo[2.2.1]heptan-1-yl)methyl)-4-((4-methoxybenzyl)amino)cyclopentane-1-carboxamide (50 mg, 84 μmol) and paraformaldehyde (3.8 mg, 0.13 mmol) in MeOH (4 mL) was added NaBH₃CN (16 mg, 0.25 mmol) in portions at 0° C. under a nitrogen atmosphere. The mixture was stirred for 16 hours at room temperature. The mixture was diluted with water (5 mL), and the aqueous phase was extracted with ethyl acetate (10 mL*3). The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 10% to 50% B in 10 min; detector: UV 254 nm) to give (3S,4R)-3-acetamido-N—((S)-(2,3-dichloro-6-fluorophenyl)(4-fluorobicyclo[2.2.1]heptan-1-yl)methyl)-4-((4-methoxybenzyl)(methyl)amino)cyclopentane-1-carboxamide (40 mg, 66 μmol) as a yellow oil. LCMS RT 0.896 min, [M+H]⁺ 608, LCMS method C.

### Step 3. Synthesis of (3S,4R)-3-acetamido-N—((S)-(2,3-dichloro-6-fluorophenyl) (4-fluoro bicyclo[2.2.1]heptan-1-yl)methyl)-4-(methylamino)cyclopentane-1-carboxamide

To a mixture of (3S,4R)-3-acetamido-N—((S)-(2,3-dichloro-6-fluorophenyl)(4-fluoro bicyclo[2.2.1]heptan-1-yl)methyl)-4-((4-methoxybenzyl)(methyl)amino)cyclopentane-1-carboxamide (40 mg, 66 μmol) in acetonitrile/H₂O (2.2 ml, 10:1) was added ceric ammonium nitrate (0.36 g, 0.66 mmol). The mixture was stirred at 20° C. for 3 hours. The reaction was quenched with water and extracted with ethyl acetate. The organic layer was washed with brine, dried over Na₂SO₄ and evaporated. The residue was purified by reverse phase flash chromatography (column: C18 silica gel; mobile phase A: water, mobile phase B: acetonitrile; gradient: 10% to 50% B in 10 min; detector: UV 254 nm) to give (3S,4R)-3-acetamido-N—((S)-(2,3-dichloro-6-fluorophenyl)(4-fluorobicyclo[2.2.1]heptan-1-yl)methyl)-4-(methylamino)cyclopentane-1-carboxamide (24 mg, 49 μmol) as a yellow oil. LCMS RT 0.755 min, [M+H]⁺ 488, LCMS method C.

### Step 4. Synthesis of (1R,3S,4R)-3-acetamido-N—((S)-(2,3-dichloro-6-fluorophenyl)(4-fluorobicyclo[2.2.1]heptan-1-yl)methyl)-4-(methylamino)cyclopentane-1-carboxamide and (1S,3S,4R)-3-acetamido-N—((S)-(2,3-dichloro-6-fluorophenyl)(4-fluorobicyclo[2.2.1]heptan-1-yl)methyl)-4-(methylamino)cyclopentane-1-carboxamide

(3S,4R)-3-acetamido-N—((S)-(2,3-dichloro-6-fluorophenyl)(4-fluorobicyclo[2.2.1]heptan-1-yl)methyl)-4-(methylamino)cyclopentane-1-carboxamide (24 mg, 49 μmol) was purified by chiral preparative HPLC (column: CHIRALPAK IE, 2*25 cm, 5 μm; mobile phase A: hexane (0.5% 2M NH₃ in MeOH), mobile phase B: EtOH:DCM 1:1; flow rate: 20 mL/min; gradient: 30% B isocratic; wavelength: 220/254 nm; RT1 (min): 8.86; RT2 (min): 10.32; sample solvent: EtOH:DCM 1:1; injection volume: 0.5 mL) to give (1R,3S,4R)-3-acetamido-N—((S)-(2,3-dichloro-6-fluorophenyl)(4-fluorobicyclo[2.2.1]heptan-1-yl)methyl)-4-(methylamino)cyclopentane-1-carboxamide and (1S,3S,4R)-3-acetamido-N—((S)-(2,3-dichloro-6-fluorophenyl)(4-fluorobicyclo[2.2.1]heptan-1-yl)methyl)-4-(methylamino)cyclopentane-1-carboxamide, both as a white amorphous solid.

Isomer 1: 2 mg, 4 μmol. LCMS RT 1.772 min). ¹HNMR (400 MHz, DMSO-d₆) δ 8.07 (d, J=8.4 Hz, 1H), 7.82 (d, J=7.2 Hz, 1H), 7.62 (dd, J=9.0, 5.1 Hz, 1H), 7.26 (t, J=9.8 Hz, 1H), 5.52 (d, J=8.1 Hz, 1H), 3.92 (td, J=7.4, 3.6 Hz, 1H), 3.57 (p, J=6.0 Hz, 1H), 3.21 (d, J=13.8 Hz, 3H), 2.90 (p, J=8.4 Hz, 1H), 2.25-2.05 (m, 1H), 1.92 (dt, J=13.6, 8.2 Hz, 1H), 1.78 (d, J=3.5 Hz, 5H), 1.73 (s, 4H), 1.71 (dd, J=12.4, 8.7 Hz, 1H), 1.70-1.55 (m, 3H), 1.50 (s, 1H).

Isomer 2: 2.9 mg, 5.9 μmol. ¹H NMR (400 MHz, DMSO-d₆) δ 8.07 (d, J=8.3 Hz, 1H), 7.82 (d, J=7.3 Hz, 1H), 7.62 (dd, J=9.0, 5.1 Hz, 1H), 7.26 (t, J=9.8 Hz, 1H), 5.52 (d, J=8.2 Hz, 1H), 3.92 (tq, J=10.6, 5.4 Hz, 1H), 3.57 (p, J=6.1 Hz, 1H), 3.19 (s, 2H), 2.90 (p, J=8.4 Hz, 1H), 2.15 (ddt, J=28.7, 14.6, 7.6 Hz, 1H), 1.98-1.82 (m, 2H), 1.78 (d, J=3.5 Hz, 5H), 1.72 (s, 2H), 1.70-1.56 (m, 5H), 1.58-1.40 (m, 1H).

### Example 30

### (1S,3S,4S)-3-acetamido-N—((S)-(3-chloro-2,6-difluorophenyl)(4-fluorobicyclo[2.2.1]heptan-1-yl)methyl)-4-hydroxycyclopentane-1-carboxamide and (1R,3S,4S)-3-acetamido-N—((S)-(3-chloro-2,6-difluorophenyl)(4-fluorobicyclo[2.2.1]heptan-1-yl)methyl)-4-hydroxycyclopentane-1-carboxamide

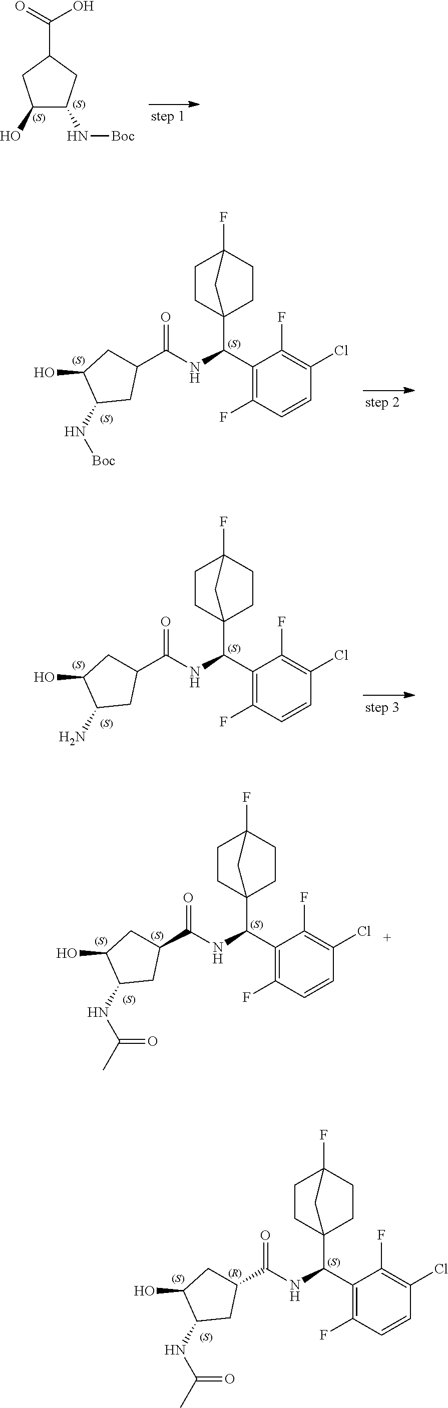

### Step 1. Synthesis of tert-butyl ((1S,2S)-4-(((S)-(3-chloro-2,6-difluorophenyl) (4-fluorobicyclo [2.2.1]heptan-1-yl) methyl) carbamoyl)-2-hydroxycyclopentyl)

(3S,4S)-3-((tert-butoxycarbonyl)amino)-4-hydroxycyclopentane-1-carboxylic acid was prepared using the same procedure in Example 31, from ethyl (3S,4R)-3-((tert-butoxycarbonyl)amino)-4-hydroxycyclopentane-1-carboxylate. To a mixture of (3S,4S)-3-((tert-butoxycarbonyl) amino)-4-hydroxycyclopentane-1-carboxylic acid (0.98 g, 4.0 mmol), (S)-(3-chloro-2,6-difluorophenyl)(4-fluorobicyclo[2.2.1]heptan-1-yl)methanamine (1.16 g, 4 mmol), NaHCO₃ (0.84 g, 0.01 mol) in DMF (10 mL) was added HATU (2.28 g, 6 mmol). The mixture was stirred for 1 h at 25° C. The reaction mixture was diluted with water (50 mL). The aqueous phase was extracted with ethyl acetate (50 ml) three times. The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 0% to 100% B in 10 min; detector: UV 220 nm) to give tert-butyl ((1S,2S)-4-(((S)-(3-chloro-2,6-difluorophenyl)(4-fluorobicyclo[2.2.1]heptan-1-yl)methyl)carbamoyl)-2-hydroxycyclopentyl)carbamate (1.03 g, 2 mmol) as an off-white amorphous solid. LCMS RT 0.972 min, [M+H]⁺ 517.40, LCMS method C.

### Step 2. Synthesis of (3S,4S)-3-amino-N—((S)-(3-chloro-2,6-difluorophenyl) (4-fluorobicyclo [2.2.1]heptan-1-yl) methyl)-4-hydroxycyclopentane-1-carboxamide

A mixture of tert-butyl ((1S,2S)-4-(((S)-(3-chloro-2,6-difluorophenyl)(4-fluorobicyclo[2.2.1]heptan-1-yl)methyl)carbamoyl)-2-hydroxycyclopentyl)carbamate (500 mg, 967 μmol) in HCl (5 mL, 4 N in MeOH) was stirred for 30 min at 25° C. Concentration in vacuo gave (3S,4S)-3-amino-N—((S)-(3-chloro-2,6-difluorophenyl)(4-fluorobicyclo[2.2.1]heptan-1-yl)methyl)-4-hydroxycyclopentane-1-carboxamide (400 mg, 960 μmol) as a white solid. LCMS RT 0.918 min, [M+H]⁺ 417.15, LCMS method B.

### Step 3. Synthesis of (1S,3S,4S)-3-acetamido-N—((S)-(3-chloro-2,6-difluorophenyl)(4-fluorobicyclo[2.2.1]heptan-1-yl)methyl)-4-hydroxycyclopentane-1-carboxamide and (1R,3S,4S)-3-acetamido-N—((S)-(3-chloro-2,6-difluorophenyl)(4-fluorobicyclo[2.2.1]heptan-1-yl)methyl)-4-hydroxycyclopentane-1-carboxamide

To mixture of (3S,4S)-3-amino-N—((S)-(3-chloro-2,6-difluorophenyl)(4-fluorobicyclo[2.2.1]heptan-1-yl)methyl)-4-hydroxycyclopentane-1-carboxamide (390 mg, 936 μmol), acetic acid (169 mg, 2.81 mmol), TEA (283 mg, 2.81 mmol) in DMF (1 mL) was added T₃P (446 mg, 1.40 mmol). The mixture was stirred for 1 h at 25° C. The reaction mixture was diluted with water (10 mL), and the aqueous phase was extracted with ethyl acetate (20 ml) three times. The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 0% to 100% B in 10 min; detector: UV 220 nm) to give an amorphous off-white solid. LCMS RT 0.721 min, [M+H]⁺ 517, LCMS method C.

The product was further purified by chiral preparative HPLC (column: CHIRALPAK ID, 2*25 cm, 5 μm; mobile phase A: hexane (0.5% 2M NH₃ in MeOH), mobile phase B: EtOH:DCM 1:1; flow rate: 20 mL/min; gradient: 15% B isocratic; wavelength: 220/254 nm; RT1 (min): 7.41; RT2 (min): 9.34; sample solvent: EtOH:DCM 1:1; injection volume: 0.6 mL) to give (1S,3S,4S)-3-acetamido-N—((S)-(3-chloro-2,6-difluorophenyl)(4-fluorobicyclo[2.2.1]heptan-1-yl)methyl)-4-hydroxycyclopentane-1-carboxamide and (1R,3S,4S)-3-acetamido-N—((S)-(3-chloro-2,6-difluorophenyl)(4-fluorobicyclo[2.2.1]heptan-1-yl)methyl)-4-hydroxycyclopentane-1-carboxamide, both as an off-white amorphous solid.

Isomer 1: 27.0 mg, 58.6 μmol. ¹H NMR (400 MHz, DMSO-d6) δ 8.24 (d, J=8.3 Hz, 1H), 7.79 (d, J=6.9 Hz, 1H), 7.57 (td, J=8.6, 5.4 Hz, 1H), 7.20-7.11 (m, 1H), 5.26 (d, J=8.3 Hz, 1H), 5.13 (d, J=4.6 Hz, 1H), 3.89-3.70 (m, 2H), 2.88 (p, J=8.1 Hz, 1H), 2.12-2.01 (m, 1H), 1.88 (dt, J=14.4, 7.6 Hz, 1H), 1.78 (s, 6H), 1.71 (d, J=13.5 Hz, 4H), 1.56 (ddd, J=13.7, 11.1, 6.7 Hz, 3H), 1.44 (q, J=7.4, 5.2 Hz, 2H). LCMS RT 0.878 min, [M+H]⁺ 459.35, LCMS method D.

Isomer 2: 15.5 mg, 33.4 μmol. ¹H NMR (400 MHz, DMSO-d6) δ 8.23 (d, J=8.3 Hz, 1H), 7.79 (d, 0.1=6.7 Hz, 1H), 7.57 (td, J=8.7, 5.4 Hz, 1H), 7.16 (t, J=9.4 Hz, 1H), 5.26 (d, J=8.2 Hz, 1H), 5.10 (d, J=4.7 Hz, 1H), 3.79 (dp, J=19.7, 5.9 Hz, 2H), 2.88 (p, J=8.3 Hz, 1H), 2.06-1.95 (m, 2H), 1.79 (s, 7H), 1.71 (d, J=9.3 Hz, 4H), 1.63-1.54 (m, 2H), 1.45 (p, J=6.6 Hz, 3H). LCMS RT 0.888 min, [M+H]⁺ 459.35, LCMS method D.

### Example 31

### (1S,2R,3S,4S)-4-acetamido-N—((S)-(3-chloro-2,6-difluorophenyl)(4-fluorobicyclo[2.2.1]heptan-1-yl)methyl)-2,3-dihydroxycyclopentane-1-carboxamide and (1S,2S,3R,4S)-4-acetamido-N—((S)-(3-chloro-2,6-difluorophenyl)(4-fluorobicyclo[2.2.1]heptan-1-yl)methyl)-2,3-dihydroxycyclopentane-1-carboxamide

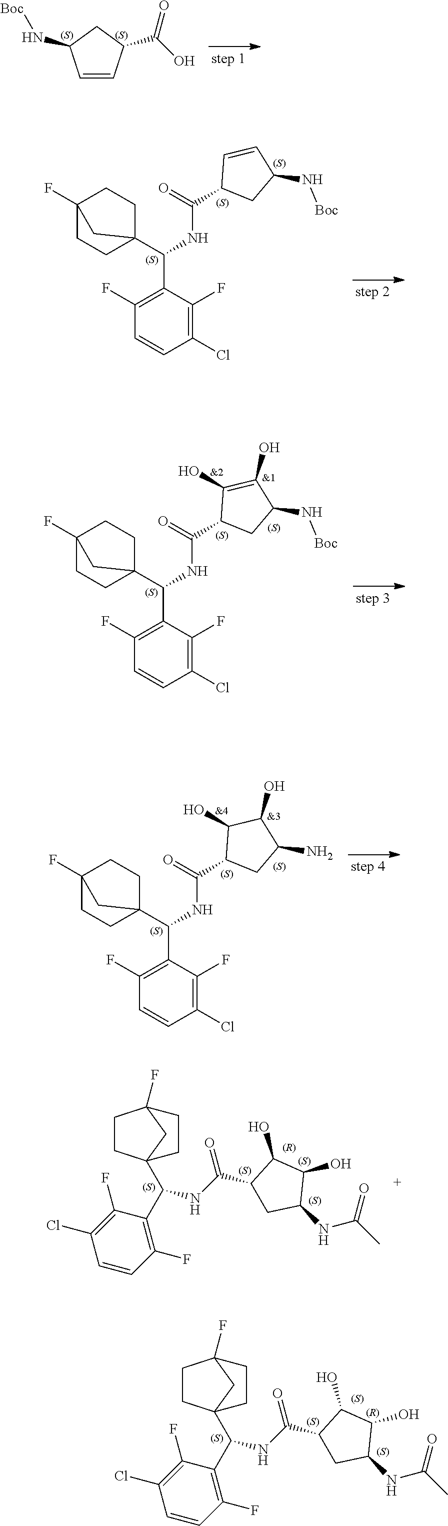

### Step 1. Synthesis of tert-butyl ((1S,4S)-4-(((S)-(3-chloro-2,6-difluorophenyl)(4-fluorobicyclo[2.2.1]heptan-1-yl)methyl)carbamoyl)cyclopent-2-en-1-yl)carbamate

To a solution of (1S,4S)-4-((tert-butoxycarbonyl)amino)cyclopent-2-ene-1-carboxylic acid (150 mg, 660 μmol), (S)-(3-chloro-2,6-difluorophenyl)(4-fluorobicyclo[2.2.1]heptan-1-yl)methanamine (191 mg, 660 μmol) and NaHCO₃ (277 mg, 3.30 mmol) in DMF (2 mL) was added HATU (318 mg, 1.32 mmol). The mixture was stirred for 1 hour at 25° C. The reaction mixture was diluted with water (10 mL), and the aqueous phase was extracted with ethyl acetate (15 mL) three times. The combined organic layers were washed with brine, dried over sodium sulfate, filtered and concentrated in vacuo. The resulting crude material was purified by preparative HPLC (column: XBridge Prep OBD C18 Column, 30*150 mm, 5 μm; mobile phase A: water (10 mM NH₄HCO₃), mobile phase B: acetonitrile; flow rate: 60 mL/min; gradient: 56% B to 79% B in 8 min, then 79% B; wavelength: 254 nm; RT1 (min): 7.63; injection volume: 0.8 mL). Lyophilization yielded tert-butyl ((1S,4S)-4-(((S)-(3-chloro-2,6-difluorophenyl)(4-fluorobicyclo[2.2.1]heptan-1-yl)methyl)carbamoyl)cyclopent-2-en-1-yl)carbamate (190 mg, 381 μmol) as an off-white amorphous solid. LCMS RT 1.217 min, [M+H]⁺ 499.10, LCMS method B.

### Step 2. Synthesis of tert-butyl ((1S,2RS,3SR,4S)-4-(((S)-(3-chloro-2,6-difluorophenyl)(4-fluorobicyclo[2.2.1]heptan-1-yl)methyl)carbamoyl)-2,3-dihydroxycyclopentyl)carbamate

A mixture of tert-butyl ((1S,4S)-4-(((S)-(3-chloro-2,6-difluorophenyl)(4-fluorobicyclo[2.2.1]heptan-1-yl)methyl)carbamoyl)cyclopent-2-en-1-yl)carbamate (180 mg, 361 μmol), NMO (10.8 mg, 361 μmol), K₂OSO₄·2H₂O (11.1 mg, 36.1 μmol) in DCM (2 mL) was stirred for 1 hour at 25° C. The reaction mixture was diluted with water (10 mL), and the aqueous phase was extracted with ethyl acetate (15 mL) three times. The combined organic layers were washed with brine, dried over sodium sulfate, filtered and concentrated in vacuo. The resulting crude material was purified by preparative HPLC (column: XBridge Prep OBD C18 Column, 30*150 mm, 5 μm; mobile phase A: water (10 mM NH₄HCO₃), mobile phase B: acetonitrile; flow rate: 60 mL/min; gradient: 56% B to 79% B in 8 min, then 79% B; wavelength: 254 nm; RT (min): 7.63; injection volume: 0.8 mL) to give tert-butyl ((1S,2RS,3SR,4S)-4-(((S)-(3-chloro-2,6-difluorophenyl)(4-fluorobicyclo[2.2.1]heptan-1-yl)methyl)carbamoyl)-2,3-dihydroxycyclopentyl)carbamate (140 mg, 263 μmol) as an off-white amorphous solid. LCMS RT 1.105 min, [M+H]⁺ 533.10, LCMS method C.

### Step 3. Synthesis of (1S,2RS,3SR,4S)-4-amino-N—((S)-(3-chloro-2,6-difluorophenyl)(4-fluorobicyclo[2.2.1]heptan-1-yl)methyl)-2,3-dihydroxycyclopentane-1-carboxamide

A mixture of tert-butyl ((1S,2RS,3SR,4S)-4-(((S)-(3-chloro-2,6-difluorophenyl)(4-fluorobicyclo[2.2.1]heptan-1-yl)methyl)carbamoyl)-2,3-dihydroxycyclopentyl)carbamate (130 mg, 244 μmol) in HCl (3 mL, 4 N in MeOH) was stirred for 2 hours at 25° C. The mixture was concentrated in vacuo to give (1S,2RS,3SR,4S)-4-amino-N—((S)-(3-chloro-2,6-difluorophenyl)(4-fluorobicyclo[2.2.1]heptan-1-yl)methyl)-2,3-dihydroxycyclopentane-1-carboxamide (150 mg) as a white amorphous solid. LCMS RT 0.913 min, [M+H]⁺ 433.30, LCMS method C.

### Step 4. Synthesis of (1S,2R,3S,4S)-4-acetamido-N—((S)-(3-chloro-2,6-difluorophenyl)(4-fluorobicyclo[2.2.1]heptan-1-yl)methyl)-2,3-dihydroxycyclopentane-1-carboxamide and (1S,2S,3R,4S)-4-acetamido-N—((S)-(3-chloro-2,6-difluorophenyl)(4-fluorobicyclo[2.2.1]heptan-1-yl)methyl)-2,3-dihydroxycyclopentane-1-carboxamide

To a solution of (1S,2RS,3SR,4S)-4-amino-N—((S)-(3-chloro-2,6-difluorophenyl)(4-fluorobicyclo[2.2.1]heptan-1-yl)methyl)-2,3-dihydroxycyclopentane-1-carboxamide (140 mg, 323 μmol), acetic acid (25.2 mg, 420 μmol) and NaHCO₃ (136 mg, 1.62 mmol) in DMF (2 mL) was added HATU (160 mg, 420 μmol). The mixture was stirred for 12 hours at 25° C. The reaction mixture was diluted with water (10 mL), and the aqueous phase was extracted with ethyl acetate (20 mL*3). The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 10% to 80% B in 25 min; detector: UV 254 nm) to give a white amorphous solid. LCMS RT 0.681 min, [M+H]⁺ 475.15, LCMS method B.

The material was further purified by preparative chiral HPLC (Column: CHIRALPAK IH3; mobile phase A: hexane (0.2% diethylamine), mobile phase B: EtOH:DCM 1:1); gradient: A:B 80:20 isocratic; flow rate: 1 mL/min; injection volume: 3 mL) to give (1 S,2R,3S,4S)-4-acetamido-N—((S)-(3-chloro-2,6-difluorophenyl)(4-fluorobicyclo[2.2.1]heptan-1-yl)methyl)-2,3-dihydroxycyclopentane-1-carboxamide and (1S,2S,3R,4S)-4-acetamido-N—((S)-(3-chloro-2,6-difluorophenyl)(4-fluorobicyclo[2.2.1]heptan-1-yl)methyl)-2,3-dihydroxycyclopentane-1-carboxamide, both as a white amorphous solid.

Isomer 1: 23.7 mg, 49.9 μmol. LCMS RT 0.950 min, [M+H]⁺ 475.10, LCMS method B. ¹HNMR (400 MHz, DMSO-d6) δ 8.16 (d, J=8.5 Hz, 11H), 7.87 (d, J=7.7 Hz, 1H), 7.69-7.48 (m, 1H), 7.16 (t, J=9.4 Hz, 1H), 5.31 (d, J=8.4 Hz, 1H), 4.93-4.52 (m, 2H), 3.86 (dd, J=7.9, 4.2 Hz, 2H), 3.56 (d, J=4.9 Hz, 1H), 2.94-2.63 (m, 1H), 2.05 (dt, J=13.2, 8.6 Hz, 1H), 1.91-1.53 (m, 11H), 1.44 (d, J=10.8 Hz, 2H), 1.27-1.07 (m, 1H).

Isomer 2: 2.8 mg, 5.9 μmol. LCMS RT 0.806 min, [M+H]⁺ 475.00, LCMS method C. ¹H NMR (400 MHz, DMSO-d6) δ 8.53 (d, J=8.4 Hz, 1H), 7.58 (t, J=5.8 Hz, 2H), 7.16 (t, J=9.3 Hz, 1H), 5.32 (d, J=8.3 Hz, 1H), 5.13 (d, J=7.9 Hz, 1H), 4.97 (d, J=5.3 Hz, 1H), 4.14 (d, J=8.6 Hz, 1H), 4.04-3.89 (m, 1H), 3.67-3.59 (m, 1H), 2.96 (q, J=8.4 Hz, 1H), 1.75 (d, J=31.7 Hz, 13H), 1.51-1.34 (m, 1H).

### Example 32

### (1S,4S)-4-acetamido-N—((S)-(3-chloro-2-fluoro-5-hydroxyphenyl)(4-fluorobicyclo[2.2.1]heptan-1-yl)methyl)-3,3-difluorocyclopentane-1-carboxamide, (1R,4S)-4-acetamido-N—((S)-(3-chloro-2-fluoro-5-hydroxyphenyl)(4-fluorobicyclo[2.2.1]heptan-1-yl)methyl)-3,3-difluorocyclopentane-1-carboxamide, (1S,4R)-4-acetamido-N—((S)-(3-chloro-2-fluoro-5-hydroxyphenyl)(4-fluorobicyclo[2.2.1]heptan-1-yl)methyl)-3,3-difluorocyclopentane-1-carboxamide and (1R,4R)-4-acetamido-N—((S)-(3-chloro-2-fluoro-5-hydroxyphenyl)(4-fluorobicyclo[2.2.1]heptan-1-yl)methyl)-3,3-difluorocyclopentane-1-carboxamide

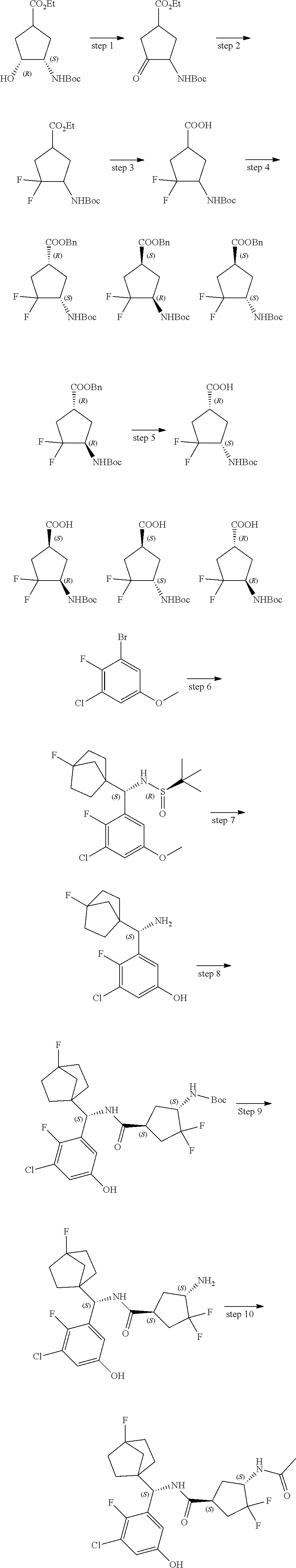

### Step 1. Synthesis of ethyl 3-((tert-butoxycarbonyl)amino)-4-oxocyclopentane-1-carboxylate

To a mixture of ethyl (3S,4R)-3-((tert-butoxycarbonyl)amino)-4-hydroxycyclopentane-1-carboxylate (130 g, 475 mmol) and 4 Å molecular sieves (40.0 g) in DCM (1.30 L) was added PCC (133 g, 618 mmol) at 25° C. The mixture was stirred at 25° C. for 1 hour. The mixture was diluted with MTBE (4.50 L) and filtered through celite under reduced pressure. The filtrate was concentrated under reduced pressure. The residue was purified by column chromatography (SiO₂, petroleum ether:ethyl acetate 20:1 to 0:1) to give ethyl 3-((tert-butoxycarbonyl)amino)-4-oxocyclopentane-1-carboxylate (71.1 g, 262 mmol) as a yellow oil. ¹H NMR: (400 MHz, DMSO-d₆) δ 7.09 (dd, J=8.0, 20.0 Hz, 1H), 4.10 (q, J=6.8 Hz, 2H), 3.99-3.77 (m, 1H), 3.26-3.01 (m, 1H), 2.48-2.40 (m, 1H), 2.39-2.20 (m, 2H), 2.12-1.81 (m, 1H), 1.37 (s, 9H), 1.19 (t, J=7.2 Hz, 3H).

### Step 2. Synthesis of ethyl 4-((tert-butoxycarbonyl)amino)-3,3-difluorocyclopentane-1-carboxylate

To a mixture of ethyl 3-((tert-butoxycarbonyl)amino)-4-oxocyclopentane-1-carboxylate (31.7 g, 117 mmol) in DCM (317 mL) was added DAST (77.3 mL, 585 mmol) at 0° C. under N₂. The mixture was warmed to 25° C. and stirred at 25° C. for 2 hours. The mixture was cooled to 0° C. and quenched with MeOH (150 mL). The mixture was stirred at 25° C. for 12 hours and concentrated under reduced pressure. The residue was purified by column chromatography (SiO₂, petroleum ether:ethyl acetate 50:1 to 3:1) to give ethyl 4-((tert-butoxycarbonyl)amino)-3,3-difluorocyclopentane-1-carboxylate (27.5 g, 93.8 mmol) as a brown oil. ¹H NMR: (400 MHz, DMSO-d₆) δ 7.19 (dd, J=9.2 Hz, 12.8 Hz, 1H), 4.20-3.98 (m, 3H), 3.13-2.95 (m, 1H), 2.45-2.08 (m, 3H), 1.95-1.69 (m, 1H), 1.39 (s, 9H), 1.21-1.16 (m, 3H).

### Step 3. Synthesis of 4-((tert-butoxycarbonyl)amino)-3,3-difluorocyclopentane-1-carboxylic acid

To a mixture of ethyl 4-((tert-butoxycarbonyl)amino)-3,3-difluorocyclopentane-1-carboxylate (28.5 g, 97.2 mmol) in MeOH (427 mL) and H₂O (140 mL) was added LiOH H₂O (20.4 g, 486 mmol) at 0° C. The mixture was warmed to 25° C. and stirred at 25° C. for 2 hours. The mixture was concentrated under reduced pressure to remove most of MeOH. The residue was diluted with H₂O (300 mL). The mixture's pH was adjusted to 4 with saturated citric acid aqueous solution and extracted with DCM (300 mL*3). The combined organic layer was washed with brine (100 mL), dried over Na₂SO₄, filtered and concentrated under reduced pressure to obtain 4-((tert-butoxycarbonyl)amino)-3,3-difluorocyclopentane-1-carboxylic acid (25.7 g, crude) as a brown oil.

### Step 4. Synthesis of benzyl (1R,4S)-4-((tert-butoxycarbonyl)amino)-3,3-difluorocyclopentane-1-carboxylate, benzyl (1S,4S)-4-((tert-butoxycarbonyl)amino)-3,3-difluorocyclopentane-1-carboxylate, benzyl (1S,4R)-4-((tert-butoxycarbonyl)amino)-3,3-difluorocyclopentane-1-carboxylate and benzyl (1R,4R)-4-((tert-butoxycarbonyl)amino)-3,3-difluorocyclopentane-1-carboxylate

To a mixture of 4-((tert-butoxycarbonyl)amino)-3,3-difluorocyclopentane-1-carboxylic acid (25.7 g, 96.9 mmol) in DMF (260 mL) was added K₂CO₃ (26.8 g, 194 mmol) and BnBr (19.9 g, 116 mmol) at 25° C. The mixture was stirred at 25° C. for 2 hours. The mixture was poured into H₂O (1.00 L) under stirring. The mixture was extracted with ethyl acetate (500 mL*3), then combined organic phase was dried with Na₂SO₄, filtered and concentrated under reduced pressure. The residue was purified by column chromatography (SiO₂, petroleum ether:ethyl acetate 10:1 to 0:1) to give the product (34.0 g, crude) as a white solid.

The product (33.0 g) was purified by reverse phase HPLC (mobile phase A: 0.1% NH₄OH in water, mobile phase B: acetonitrile). The collected fractions were concentrated under reduced pressure to remove acetonitrile. The remaining aqueous solution was extracted with ethyl acetate (500 mL*3). The combined organic phase was dried over Na₂SO₄, filtered and concentrated under reduced pressure to give a solid (30.0 g). The residue was purified by chiral SFC (column: Daicel Chiralcel OJ 250 mm*50 mm, 10 μm; mobile phase: 12% isopropanol in hexane) to obtain peak 1 and peak 2.

Peak 1, after concentration, was further purified by chiral SFC (column: Daicel Chiralpak IG 250 mm*50 mm, 10 μm; mobile phase: 20% MeOH in 0.1% NH₄OH]) to obtain peak 3 and peak 4.

Peak 3 was concentrated under reduced pressure to give a white solid (5.20 g, 14.6 mmol). ¹⁹F NMR (376 MHz, DMSO-d₆) S -101.23 ppm, −101.83 ppm, −107.06 ppm, −107.66 ppm. ¹H NMR: (400 MHz, DMSO-d₆) δ 7.41-7.31 (m, 5H), 7.22 (d, J=7.6 Hz, 1H), 5.12 (s, 2H), 4.26-4.05 (m, 1H), 3.27-3.09 (m, 1H), 2.46-2.32 (m, 2H), 2.26-2.12 (m, 1H), 1.98-1.83 (m, 1H), 1.39 (s, 9H).

Peak 4 was concentrated under reduced pressure to give a yellow solid (9.00 g, 25.3 mmol). ¹⁹F NMR: (376 MHz, DMSO-d₆) δ −101.23 ppm, −101.83 ppm, −107.07 ppm, −107.67 ppm. ¹H NMR: (400 MHz, DMSO-d₆) δ 7.41-7.31 (m, 5H), 7.22 (d, J=7.2 Hz, 1H), 5.12 (s, 2H), 4.26-4.05 (m, 1H), 3.27-3.09 (m, 1H), 2.46-2.32 (m, 2H), 2.26-2.12 (m, 1H), 1.98-1.83 (m, 1H), 1.39 (s, 9H).

Peak 2, after concentration, was further purified by chiral SFC (column: Daicel Chiralpak IG (250 mm*50 mm, 10 μm); mobile phase: 15% MeOH in 0.1% NH40H) to obtain peak 5 and peak 6.

Peak 5 was concentrated under reduced pressure to give a white solid (4.30 g, 12.1 mmol). ¹⁹F NMR: (376 MHz, DMSO-d₆) δ −100.03 ppm, −100.62 ppm, −103.25 ppm, −103.85 ppm. ¹H NMR (400 MHz, DMSO-d₆) δ 7.43-7.30 (m, 5H), 7.19 (d, J=8.8 Hz, 1H), 5.12 (s, 2H), 4.24-4.06 (m, 1H), 3.11 (br s, 1H), 2.46-2.16 (m, 3H), 1.85-7.30 (m, 1H), 1.38 (s, 9H).

Peak 6 was concentrated under reduced pressure to give a yellow solid (8.90 g, 25.0 mmol). ¹⁹F NMR: (376 MHz, DMSO-d₆) δ −100.03 ppm, −100.62 ppm, −103.25 ppm, −103.85 ppm

¹H NMR: (400 MHz, DMSO-d₆) δ 7.43-7.30 (m, 5H), 7.19 (d, J=9.2 Hz, 1H), 5.12 (s, 2H), 4.25-4.02 (m, 1H), 3.10-3.00 (m, 1H), 2.48-2.17 (m, 3H), 1.88-1.74 (m, 1H), 1.38 (s, 9H).

### Step 5. Synthesis of (1R,4S)-4-((tert-butoxycarbonyl)amino)-3,3-difluorocyclopentane-1-carboxylic acid, (1S,4S)-4-((tert-butoxycarbonyl)amino)-3,3-difluorocyclopentane-1-carboxylic acid, (1S,4R)-4-((tert-butoxycarbonyl)amino)-3,3-difluorocyclopentane-1-carboxylic acid and (1R,4R)-4-((tert-butoxycarbonyl)amino)-3,3-difluorocyclopentane-1-carboxylic acid

To a solution of peak 4 in step 4 (9.00 g, 25.3 mmol) in MeOH (135 mL) was added Pd/C (1.80 g, 10%) at 25° C. under N₂. The mixture was degassed and purged with H₂ 3 times. The mixture was stirred at 25° C. for 4 hours under H₂ (50 psi). The mixture was filtered through celite under reduced pressure. The filtrate was concentrated under reduced pressure to give a white solid (6.36 g, 25.6 mmol) as a white solid. ¹⁹F NMR: (376 MHz, DMSO-d₆) δ −101.09 ppm, −101.69 ppm, −106.70 ppm, −107.30 ppm. ¹H NMR: (400 MHz, DMSO-d₆) δ 12.58 (s, 1H), 7.19 (d, J=8.8 Hz, 1H), 4.30-3.96 (m, 1H), 3.08-2.88 (m, 1H), 2.38-2.28 (m, 2H), 2.17-2.11 (m, 1H), 1.90-1.80 (m, 1H), 1.39 (s, 9H).

The other 3 isomers were synthesized similarly.

### Step 6. Synthesis of (R)—N—((S)-(3-chloro-2-fluoro-5-methoxyphenyl) (4-fluorobicyclo [2.2.1]heptan-1-yl) methyl)-2-methylpropane-2-sulfinamide

To a mixture of 1-bromo-3-chloro-2-fluoro-5-methoxybenzene (1.2 g, 5.0 mmol) in THF (12 mL) was added n-butyllithium (2.4 mL, 2.5 M in THF, 6.0 mmol) dropwise at −78° C. under a nitrogen atmosphere. The mixture was stirred for 1 h at −78° C. prior to the addition of (R)—N-((4-fluorobicyclo [2.2.1]heptan-1-yl) methylene)-2-methylpropane-2-sulfinamide (981 mg, 4.0 mmol) at −78° C. The mixture was stirred for 1 hour at −78° C. The reaction was quenched with saturated NH₄Cl (aq.) and the aqueous phase was extracted with ethyl acetate (30 mL) three times. The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 0% to 100% B in 20 min; detector: UV 220 nm) to afford (R)—N—((S)-(3-chloro-2-fluoro-5-methoxyphenyl)(4-fluorobicyclo[2.2.1]heptan-1-yl)methyl)-2-methylpropane-2-sulfinamide (1.21 g, 2.98 mmol) as a colorless oil. LCMS RT 1.118 min, [M+H]⁺ 405.90, LCMS method B.

### Step 7. (S)-3-(amino(4-fluorobicyclo[2.2.1]heptan-1-yl)methyl)-5-chloro-4-fluorophenol

A mixture of (R)—N—((S)-(3-chloro-2-fluoro-5-methoxyphenyl)(4-fluorobicyclo[2.2.1]heptan-1-yl)methyl)-2-methylpropane-2-sulfinamide (1.2 g, 3.0 mmol) in HBr (5 ml, 33% in AcOH) was stirred at 100° C. for 4 hours. The mixture was concentrated. The resulting crude material was purified by reverse phase flash chromatography (column: C18 silica gel; mobile phase A: water, mobile phase B: acetonitrile; gradient: 0% to 100% B in 10 min; detector: UV 220 nm) to give (S)-3-(amino(4-fluorobicyclo[2.2.1]heptan-1-yl)methyl)-5-chloro-4-fluorophenol (700 mg, 2.43 mmol) as an off-white solid. LCMS RT 0.917 min, [M+H]⁺ 288.05, LCMS method D.

### Step 8. Synthesis of tert-butyl ((1S,4S)-4-(((S)-(3-chloro-2-fluoro-5-hydroxyphenyl)(4-fluorobicyclo[2.2.1]heptan-1-yl)methyl)carbamoyl)-2,2-difluorocyclopentyl)carbamate

To a solution of (S)-3-(amino(4-fluorobicyclo[2.2.1]heptan-1-yl)methyl)-5-chloro-4-fluorophenol (100 mg, 348 μmol), (1S,4S)-4-((tert-butoxycarbonyl)amino)-3,3-difluorocyclopentane-1-carboxylic acid (111 mg, 417 μmol), TEA (145 μL, 1.04 mmol) in DMF (1 mL) was added T₃P (166 mg, 521 μmol) at room temperature. The resulting mixture was stirred for 2 hours at room temperature. The reaction mixture was diluted with water (15 mL), and the aqueous phase was extracted with DCM (20 mL) three times. The combined organic layers were washed with brine, dried over sodium sulfate, filtered and concentrated in vacuo. The resulting crude material was purified by reverse phase flash chromatography (acetonitrile/water) to give tert-butyl ((1S,4S)-4-(((S)-(3-chloro-2-fluoro-5-hydroxyphenyl)(4-fluorobicyclo[2.2.1]heptan-1-yl)methyl)carbamoyl)-2,2-difluorocyclopentyl)carbamate (130 mg, 69.9%) as a colorless oil. LCMS RT 0.892 min, [M+H]⁺ 535.00. LCMS method C.

### Step 9. Synthesis of (1S,4S)-4-amino-N—((S)-(3-chloro-2-fluoro-5-hydroxyphenyl)(4-fluorobicyclo[2.2.1]heptan-1-yl)methyl)-3,3-difluorocyclopentane-1-carboxamide

A mixture of tert-butyl ((1S,4S)-4-(((S)-(3-chloro-2-fluoro-5-hydroxyphenyl)(4-fluorobicyclo[2.2.1]heptan-1-yl)methyl)carbamoyl)-2,2-difluorocyclopentyl)carbamate (125 mg, 234 μmol) in HCl (4 N in MeOH, 3 mL) was stirred at room temperature for 2 hours. The mixture was concentrated. The resulting crude material was purified by reverse phase flash chromatography (column: C18 silica gel; mobile phase A: water, mobile phase B: acetonitrile; gradient: 0% to 100% B in 10 min; detector: UV 220 nm) to give (1S,4S)-4-amino-N—((S)-(3-chloro-2-fluoro-5-hydroxyphenyl)(4-fluorobicyclo[2.2.1]heptan-1-yl)methyl)-3,3-difluorocyclopentane-1-carboxamide (100 mg, 230 μmol) as a colorless oil. LCMS RT 0.717 min, [M+H]⁺ 435.00, LCMS method C.

### Step 10. Synthesis of (1S,4S)-4-acetamido-N—((S)-(3-chloro-2-fluoro-5-hydroxyphenyl)(4-fluorobicyclo[2.2.1]heptan-1-yl)methyl)-3,3-difluorocyclopentane-1-carboxamide

To a mixture of (1S,4S)-4-amino-N—((S)-(3-chloro-2-fluoro-5-hydroxyphenyl)(4-fluorobicyclo[2.2.1]heptan-1-yl)methyl)-3,3-difluorocyclopentane-1-carboxamide (50 mg, 0.11 mmol), NaHCO₃ (48 mg, 0.57 mmol) and acetic acid (8.3 mg, 0.14 mmol) in DMF (1 mL) was added HATU (66 mg, 0.17 mmol) at room temperature. The resulting mixture was stirred for 2 hours at room temperature. The reaction mixture was diluted with water (10 mL), a nd the aqueous phase was extracted with ethyl acetate (20 mL) three times. The combined organic layers were washed with brine, dried over sodium sulfate, filtered and concentrated in vacuo. The resulting crude material was purified by preparative HPLC (column: XBridge Prep OBD C18 Column, 30*150 mm, 5 μm; flow rate: 60 mL/min; gradient: 23% B to 50% B in 8 min, then 50% B; wavelength: 220 nm; RT1 (min): 7.58) to give (1S,4S)-4-acetamido-N—((S)-(3-chloro-2-fluoro-5-hydroxyphenyl)(4-fluorobicyclo[2.2.1]heptan-1-yl)methyl)-3,3-difluorocyclopentane-1-carboxamide (29.7 mg, 62.3 μmol) as an off-white amorphous solid. ¹H NMR (400 MHz, DMSO-d6) δ 9.81 (s, 1H), 8.37 (d, J=8.6 Hz, 1H), 8.08 (d, J=8.8 Hz, 1H), 6.79 (dd, J=5.8, 2.9 Hz, 1H), 6.65 (dd, J=5.4, 2.9 Hz, 1H), 5.20 (d, J=8.7 Hz, 1H), 4.48 (dt, J=18.1, 9.2 Hz, 1H), 3.08 (s, 1H), 2.37-2.17 (m, 2H), 2.04-1.94 (m, 1H), 1.86 (s, 4H), 1.81-1.64 (m, 6H), 1.62-1.52 (m, 2H), 1.36 (s, 2H). LCMS RT 0.899 min, [M−H]⁻ 475.15, LCMS method D.

Additional compounds prepared according to the methods of Examples 1-32 are listed in Table 2 below. Corresponding ¹H NMR and mass spectrometry characterization for these compounds are described in Table 1. Certain compounds in Table 2 below were prepared with other compounds whose preparation is described further below in the Examples.

TABLE 2
Additional exemplary compounds

| | | | | |
|---|---|---|---|---|
| I-83 | I-247 | I-248 | I-252 | I-253 |
| I-257 | I-258 | I-259 | I-260 | I-263 |
| I-266 | I-325 | I-327 | I-333 | I-334 |
| I-337 | I-338 | I-339 | I-340 | I-341 |
| I-342 | I-343 | I-344 | I-348 | I-349 |
| I-351 | I-352 | I-353 | I-354 | I-369 |
| I-370 | I-371 | I-372 | I-378 | I-384 |
| I-403 | I-406 | I-424 | I-427 | I-432 |
| I-445 | I-446 | I-454 | I-455 | I-460 |
| I-461 | I-466 | I-467 | I-469 | I-479 |
| I-494 | I-507 | I-510 | I-621 | I-630 |
| I-634 | I-635 | I-666 | I-670 | I-691 |
| I-692 | I-693 | I-694 | I-695 | I-696 |
| I-700 | I-701 | I-702 | I-703 | I-704 |
| I-709 | I-710 | I-711 | I-712 | I-717 |
| I-718 | I-719 | I-724 | I-725 | I-730 |
| I-794 | I-802 | I-803 | I-804 | I-806 |
| I-808 | I-817 | I-823 | I-825 | I-830 |
| I-838 | I-841 | I-842 | I-843 | I-844 |
| I-846 | I-847 | I-848 | I-849 | I-850 |
| I-851 | I-852 | I-858 | I-859 | I-860 |
| I-861 | I-862 | I-863 | I-872 | I-877 |
| I-878 | I-879 | I-882 | I-883 | I-887 |
| I-891 | I-897 | I-898 | I-900 | I-901 |
| I-902 | I-903 | I-904 | I-905 | I-906 |
| I-907 | I-908 | I-909 | I-910 | I-911 |
| I-912 | I-913 | I-914 | I-915 | I-916 |
| I-917 | I-918 | I-919 | I-925 | I-927 |
| I-929 | I-931 | I-932 | I-934 | I-935 |
| I-936 | I-937 | I-941 | I-942 | I-943 |
| I-944 | I-945 | I-946 | I-948 | I-949 |
| I-950 | I-951 | I-952 | I-953 | I-954 |
| I-955 | I-956 | I-957 | I-958 | I-959 |
| I-960 | I-961 | I-962 | I-963 | I-964 |
| I-965 | I-966 | I-967 | I-968 | I-980 |
| I-981 | I-982 | I-983 | I-984 | I-985 |
| I-986 | I-987 | I-988 | I-989 | I-992 |
| I-993 | I-996 | I-997 | I-998 | I-999 |
| I-1000 | I-1001 | I-1002 | I-1003 | I-1004 |
| I-1005 | I-1011 | I-1012 | I-1013 | I-1014 |
| I-1017 | I-1018 | I-1019 | I-1025 | I-1026 |
| I-1028 | I-1029 | I-1030 | I-1031 | I-1032 |
| I-1033 | I-1034 | I-1035 | I-1036 | I-1037 |
| I-1038 | I-1039 | I-1040 | I-1041 | I-1045 |
| I-1046 | I-1047 | I-1048 | I-1049 | I-1050 |
| I-1051 | I-1052 | I-1053 | I-1054 | I-1055 |
| I-1056 | I-1057 | I-1058 | I-1059 | I-1060 |
| I-1061 | I-1062 | I-1063 | I-1064 | I-1065 |
| I-1066 | I-1067 | I-1068 | I-1069 | I-1070 |
| I-1071 | I-1072 | I-1073 | I-1074 | I-1075 |
| I-1076 | I-1077 | I-1078 | I-1079 | I-1080 |
| I-1081 | I-1082 | I-1083 | I-1084 | I-1085 |
| I-1086 | I-1087 | I-1088 | I-1089 | I-1090 |
| I-1091 | I-1092 | I-1093 | I-1094 | I-1095 |
| I-1096 | I-1097 | I-1098 | I-1099 | I-1100 |
| I-1101 | I-1102 | I-1103 | I-1104 | I-1105 |
| I-1106 | I-1107 | I-1108 | I-1109 | I-1110 |
| I-1111 | I-1115 | I-1120 | I-1121 | I-1122 |
| I-1123 | I-1124 | I-1125 | I-1126 | I-1127 |
| I-1128 | I-1129 | I-1130 | I-1131 | I-1132 |
| I-1133 | I-1134 | I-1135 | I-1136 | I-1137 |
| I-1138 | I-1139 | I-1140 | I-1141 | I-1142 |
| I-1143 | I-1144 | I-1145 | I-1146 | I-1147 |
| I-1148 | I-1149 | I-1150 | I-1151 | I-1152 |
| I-1153 | I-1154 | I-1155 | I-1156 | I-1157 |
| I-1158 | I-1159 | I-1160 | I-1161 | I-1162 |
| I-1163 | I-1164 | I-1165 | I-1166 | I-1167 |
| I-1168 | I-1169 | I-1170 | I-1171 | I-1172 |
| I-1173 | I-1174 | I-1175 | I-1176 | I-1177 |
| I-1178 | I-1179 | I-1180 | I-1181 | I-1182 |
| I-1183 | I-1184 | I-1185 | I-1186 | I-1187 |
| I-1188 | I-1189 | I-1190 | I-1191 | I-1192 |
| I-1193 | I-1194 | I-1195 | I-1196 | I-1197 |
| I-1198 | I-1200 | I-1201 | I-1203 | I-1204 |
| I-1205 | I-1206 | I-1207 | I-1208 | I-1209 |
| I-1210 | I-1211 | I-1212 | I-1213 | I-1217 |
| I-1218 | I-1219 | I-1221 | I-1226 | I-1227 |
| I-1228 | I-1229 | I-1230 | I-1231 | I-1232 |
| I-1233 | I-1234 | I-1235 | I-1236 | I-1237 |
| I-1238 | I-1239 | I-1240 | I-1241 | I-1242 |
| I-1243 | I-1244 | I-1245 | I-1246 | I-1247 |
| I-1248 | I-1249 | I-1250 | I-1251 | I-1252 |
| I-1253 | I-1254 | I-1255 | I-1256 | I-1257 |
| I-1258 | I-1261 | I-1262 | I-1263 | I-1264 |
| I-1265 | I-1266 | I-1267 | I-1268 | I-1269 |
| I-1270 | I-1271 | I-1272 | I-1273 | I-1274 |
| I-1279 | I-1280 | I-1281 | I-1282 | I-1283 |
| I-1284 | I-1285 | I-1286 | I-1287 | I-1288 |
| I-1289 | I-1290 | I-1291 | I-1292 | I-1293 |
| I-1294 | I-1295 | I-1296 | I-1297 | I-1298 |
| I-1299 | I-1300 | I-1301 | I-1302 | I-1303 |
| I-1304 | I-1305 | I-1306 | I-1307 | I-1308 |
| I-1309 | I-1310 | I-1311 | I-1312 | I-1313 |
| I-1314 | I-1315 | I-1316 | I-1317 | I-1318 |
| I-1319 | I-1320 | I-1321 | I-1322 | I-1323 |
| I-1324 | I-1325 | I-1326 | I-1327 | I-1336 |
| I-1337 | I-1338 | I-1339 | I-1340 | I-1341 |
| I-1342 | I-1344 | I-1345 | I-1346 | I-1347 |
| I-1348 | I-1349 | I-1350 | I-1351 | I-1352 |
| I-1353 | I-1354 | I-1355 | I-1356 | I-1357 |
| I-1358 | I-1359 | I-1360 | I-1361 | I-1362 |
| I-1364 | I-1365 | I-1366 | I-1367 | I-1368 |
| I-1369 | I-1370 | I-1371 | I-1372 | I-1373 |
| I-1374 | I-1375 | I-1376 | I-1377 | I-1378 |
| I-1379 | I-1380 | I-1381 | I-1382 | I-1383 |
| I-1384 | I-1385 | I-1386 | I-1387 | I-1388 |
| I-1389 | I-1390 | I-1391 | I-1392 | I-1393 |
| I-1394 | I-1395 | I-1396 | I-1397 | I-1398 |
| I-1399 | I-1400 | I-1401 | I-1402 | I-1403 |
| I-1404 | I-1405 | I-1406 | I-1407 | I-1408 |
| I-1409 | I-1410 | I-1411 | I-1412 | I-1413 |
| I-1414 | I-1415 | I-1416 | I-1417 | I-1418 |
| I-1419 | I-1420 | I-1421 | I-1422 | I-1423 |
| I-1424 | I-1425 | I-1426 | I-1427 | I-1428 |
| I-1429 | I-1430 | I-1431 | I-1432 | I-1433 |
| I-1434 | I-1435 | I-1436 | I-1437 | I-1438 |
| I-1439 | I-1440 | I-1441 | I-1442 | I-1443 |
| I-1444 | I-1445 | I-1446 | I-1447 | I-1448 |
| I-1449 | I-1450 | I-1451 | I-1452 | I-1453 |
| I-1454 | I-1455 | I-1456 | I-1457 | I-1458 |
| I-1459 | I-1460 | I-1461 | I-1462 | I-1463 |
| I-1464 | I-1465 | I-1466 | I-1467 | I-1468 |
| I-1469 | I-1470 | I-1471 | I-1472 | I-1473 |
| I-1474 | I-1475 | I-1476 | I-1477 | I-1478 |
| I-1479 | I-1480 | I-1481 | I-1482 | I-1483 |
| I-1484 | I-1485 | I-1486 | I-1487 | I-1488 |
| I-1489 | I-1490 | I-1491 | I-1492 | I-1494 |
| I-1495 | I-1496 | I-1497 | I-1498 | I-1499 |
| I-1500 | I-1501 | I-1502 | I-1503 | I-1504 |
| I-1505 | I-1517 | I-1518 | I-1519 | I-1520 |
| I-1521 | I-1522 | I-1523 | I-1524 | I-1525 |
| I-1526 | I-1527 | I-1528 | I-1529 | I-1530 |
| I-1531 | I-1532 | I-1533 | I-1534 | I-1535 |
| I-1536 | I-1537 | I-1538 | I-1539 | I-1540 |
| I-1541 | I-1542 | I-1543 | I-1544 | I-1545 |
| I-1546 | I-1547 | I-1548 | I-1549 | I-1550 |
| I-1551 | I-1552 | I-1553 | I-1554 | I-1555 |
| I-1556 | I-1557 | I-1558 | I-1559 | I-1560 |
| I-1561 | I-1562 | I-1563 | I-1564 | I-1565 |
| I-1566 | I-1567 | I-1568 | I-1569 | I-1570 |
| I-1571 | I-1572 | I-1573 | I-1574 | I-1575 |
| I-1576 | I-1577 | I-1578 | I-1579 | I-1580 |
| I-1581 | I-1582 | I-1583 | I-1584 | I-1585 |
| I-1586 | I-1587 | I-1588 | I-1589 | I-1590 |
| I-1591 | I-1592 | I-1593 | I-1594 | I-1595 |
| I-1596 | I-1597 | I-1598 | I-1599 | I-1600 |
| I-1601 | I-1602 | I-1603 | I-1604 | I-1605 |
| I-1606 | I-1607 | I-1608 | I-1609 | I-1610 |
| I-1611 | I-1612 | I-1613 | I-1614 | I-1615 |
| I-1616 | I-1617 | I-1618 | I-1619 | I-1620 |
| I-1621 | I-1622 | I-1623 | I-1624 | I-1625 |
| I-1626 | I-1627 | I-1628 | I-1629 | I-1630 |
| I-1631 | I-1632 | I-1633 | I-1634 | I-1635 |
| I-1636 | I-1637 | I-1638 | I-1639 | I-1643 |
| I-1644 | I-1645 | I-1646 | I-1647 | I-1648 |
| I-1649 | I-1650 | I-1652 | I-1653 | I-1654 |
| I-1655 | I-1656 | I-1657 | I-1658 | I-1659 |
| I-1660 | I-1661 | I-1662 | I-1663 | I-1664 |
| I-1665 | I-1666 | I-1667 | I-1668 | I-1669 |
| I-1670 | I-1671 | I-1672 | I-1673 | I-1674 |
| I-1675 | I-1676 | I-1677 | I-1679 | I-1680 |
| I-1681 | I-1682 | I-1683 | I-1684 | I-1686 |
| I-1687 | I-1688 | I-1689 | I-1690 | I-1691 |
| I-1692 | I-1693 | I-1694 | I-1695 | I-1696 |
| I-1697 | I-1698 | I-1699 | I-1700 | I-1701 |
| I-1702 | I-1703 | I-1704 | I-1705 | I-1706 |
| I-1707 | I-1708 | I-1709 | I-1710 | I-1711 |
| I-1712 | I-1713 | I-1714 | I-1715 | I-1716 |
| I-1717 | I-1718 | I-1719 | I-1720 | I-1721 |
| I-1722 | I-1723 | I-1724 | I-1725 | I-1726 |
| I-1727 | I-1728 | I-1729 | I-1730 | I-1731 |
| I-1732 | I-1733 | I-1734 | I-1735 | I-1736 |
| I-1737 | I-1738 | I-1739 | I-1740 | I-1741 |
| I-1742 | I-1743 | I-1744 | I-1745 | I-1746 |
| I-1747 | I-1748 | I-1749 | I-1750 | I-1751 |
| I-1752 | I-1753 | I-1754 | I-1755 | I-1756 |
| I-1757 | I-1758 | I-1759 | I-1760 | I-1761 |
| I-1762 | I-1763 | I-1764 | I-1765 | I-1766 |
| I-1767 | I-1768 | I-1769 | I-1770 | I-1771 |
| I-1772 | I-1773 | I-1774 | I-1775 | I-1776 |
| I-1777 | I-1778 | I-1779 | I-1780 | I-1781 |
| I-1782 | I-1783 | I-1784 | I-1785 | I-1786 |
| I-1787 | I-1788 | I-1789 | I-1791 | I-1792 |
| I-1793 | I-1794 | I-1795 | I-1796 | I-1797 |
| I-1798 | I-1799 | I-1800 | I-1801 | I-1814 |
| I-1815 | I-1828 | I-1829 | I-1833 | I-1834 |
| I-1835 | I-1836 | I-1837 | I-1838 | I-1840 |
| I-1841 | I-1842 | I-1843 | I-1845 | I-1846 |
| I-1847 | I-1848 | I-1849 | I-1850 | I-1851 |
| I-1852 | I-1854 | I-1855 | I-1856 | I-1857 |
| I-1858 | I-1859 | I-1860 | I-1861 | I-1862 |
| I-1863 | I-1864 | I-1865 | I-1866 | I-1867 |
| I-1868 | I-1869 | I-1870 | I-1871 | I-1873 |
| I-1874 | I-1875 | I-1876 | I-1877 | I-1878 |
| I-1879 | I-1880 | I-1881 | I-1882 | I-1883 |
| I-1884 | I-1885 | I-1886 | I-1887 | I-1888 |
| I-1889 | I-1890 | I-1891 | I-1892 | I-1893 |
| I-1895 | I-1896 | I-1897 | I-1898 | I-1899 |
| I-1900 | I-1901 | I-1902 | I-1903 | I-1904 |
| I-1906 | I-1907 | I-1908 | I-1909 | I-1910 |
| I-1911 | I-1912 | I-1913 | I-1914 | I-1916 |
| I-1917 | I-1918 | I-1921 | I-1922 | I-1923 |
| I-1924 | I-1925 | I-1926 | I-1927 | I-1928 |
| I-1929 | I-1930 | I-1931 | I-1932 | I-1934 |
| I-1935 | I-1936 | I-1937 | I-1938 | I-1939 |
| I-1940 | I-1941 | I-1942 | I-1943 | I-1944 |
| I-1945 | I-1946 | I-1947 | I-1948 | I-1949 |
| I-1950 | I-1951 | I-1952 | I-1953 | I-1954 |
| I-1955 | I-1956 | I-1957 | I-1961 | I-1962 |
| I-1963 | I-1964 | I-1965 | I-1966 | I-1967 |
| I-1968 | I-1970 | I-1971 | I-1972 | I-1973 |
| I-1974 | I-1975 | I-1976 | I-1977 | I-1978 |
| I-1979 | I-1980 | I-1981 | I-1984 | I-1985 |
| I-1986 | I-1987 | I-1988 | I-1989 | I-1990 |
| I-1991 | I-1992 | I-1993 | I-1994 | I-1995 |
| I-1996 | I-1997 | I-1998 | I-1999 | I-2000 |
| I-2001 | I-2002 | I-2003 | I-2004 | I-2005 |
| I-2006 | I-2007 | I-2008 | I-2010 | I-2011 |
| I-2012 | I-2013 | I-2018 | I-2019 | I-2020 |
| I-2021 | I-2030 | I-2031 | I-2032 | I-2033 |
| I-2034 | I-2035 | I-2036 | I-2040 | I-2041 |
| I-2042 | I-2043 | I-2044 | I-2045 | I-2046 |
| I-2047 | I-2048 | I-2049 | I-2050 | I-2051 |
| I-2052 | I-2053 | I-2054 | I-2056 | I-2057 |
| I-2059 | I-2060 | I-2061 | I-2062 | I-2063 |
| I-2064 | I-2065 | I-2066 | I-2067 | I-2068 |
| I-2069 | I-2070 | I-2071 | I-2072 | I-2073 |
| I-2074 | I-2075 | I-2076 | I-2077 | I-2078 |
| I-2079 | I-2080 | I-2081 | I-2082 | I-2083 |
| I-2084 | I-2085 | I-2086 | I-2087 | I-2088 |
| I-2089 | I-2090 | I-2091 | I-2092 | I-2095 |
| I-2096 | I-2097 | I-2098 | I-2099 | I-2100 |
| I-2101 | I-2102 | I-2103 | I-2104 | I-2105 |
| I-2106 | I-2107 | I-2109 | I-2110 | I-2112 |
| I-2113 | I-2114 | I-2115 | I-2116 | I-2117 |
| I-2118 | I-2119 | I-2129 | I-2131 | I-2133 |
| I-2134 | I-2136 | I-2137 | I-2138 | I-2139 |
| I-2140 | I-2141 | I-2142 | I-2143 | I-2144 |
| I-2145 | I-2146 | I-2147 | I-2148 | I-2149 |
| I-2150 | I-2154 | I-2155 | I-2157 | I-2158 |
| I-2161 | I-2162 | I-2163 | I-2164 | I-2165 |
| I-2166 | I-2167 | I-2168 | I-2169 | I-2170 |
| I-2171 | I-2172 | I-2173 | I-2174 | I-2175 |
| I-2176 | I-2177 | I-2178 | I-2179 | I-2180 |
| I-2181 | I-2182 | I-2183 | I-2184 | I-2185 |
| I-2186 | I-2187 | I-2188 | I-2189 | I-2190 |
| I-2191 | I-2192 | I-2193 | I-2194 | I-2195 |
| I-2196 | I-2197 | I-2198 | I-2199 | I-2200 |
| I-2201 | I-2202 | I-2203 | I-2204 | I-2205 |
| I-2206 | I-2207 | I-2208 | I-2209 | I-2210 |
| I-2211 | I-2212 | I-2213 | I-2214 | I-2215 |
| I-2216 | I-2217 | I-2218 | I-2219 | I-2220 |
| I-2222 | I-2223 | I-2224 | I-2225 | I-2226 |
| I-2227 | I-2228 | I-2229 | I-2230 | I-2231 |
| I-2232 | I-2233 | I-2234 | I-2235 | I-2236 |
| I-2237 | I-2238 | I-2239 | I-2240 | I-2241 |
| I-2242 | I-2243 | I-2244 | I-2245 | I-2246 |
| I-2247 | I-2248 | I-2249 | I-2250 | I-2251 |
| I-2252 | I-2253 | I-2254 | I-2255 | I-2256 |
| I-2257 | I-2258 | I-2259 | I-2260 | I-2261 |
| I-2262 | I-2263 | I-2264 | I-2265 | I-2266 |
| I-2267 | I-2268 | I-2269 | I-2271 | I-2272 |
| I-2274 | I-2275 | I-2276 | I-2277 | I-2278 |
| I-2279 | I-2280 | I-2281 | I-2282 | I-2283 |
| I-2284 | I-2285 | I-2286 | I-2287 | I-2288 |
| I-2289 | I-2290 | I-2291 | I-2292 | I-2293 |
| I-2294 | I-2295 | I-2296 | I-2297 | I-2298 |
| I-2299 | I-2300 | I-2301 | I-2302 | I-2303 |
| I-2304 | I-2305 | I-2306 | I-2307 | I-2308 |
| I-2309 | I-2310 | I-2311 | I-2313 | I-2314 |
| I-2316 | I-2317 | I-2318 | I-2319 | I-2320 |
| I-2322 | I-2323 | I-2325 | I-2326 | I-2327 |
| I-2328 | I-2329 | I-2330 | I-2331 | I-2332 |
| I-2333 | I-2334 | I-2335 | I-2336 | I-2337 |
| I-2338 | I-2339 | I-2340 | I-2341 | I-2342 |
| I-2344 | I-2345 | I-2346 | I-2348 | I-2349 |
| I-2350 | I-2351 | I-2352 | I-2353 | I-2354 |
| I-2355 | I-2356 | I-2357 | I-2358 | I-2359 |
| I-2360 | I-2361 | I-2362 | I-2363 | I-2364 |
| I-2365 | I-2366 | I-2367 | I-2368 | I-2369 |
| I-2370 | I-2371 | I-2372 | I-2373 | I-2374 |
| I-2375 | I-2376 | I-2377 | I-2378 | I-2379 |
| I-2380 | I-2381 | I-2382 | I-2383 | I-2384 |
| I-2385 | I-2386 | I-2387 | I-2388 | I-2389 |
| I-2390 | I-2391 | I-2392 | I-2393 | I-2394 |
| I-2395 | I-2396 | I-2397 | I-2398 | I-2399 |
| I-2400 | I-2401 | I-2402 | I-2403 | I-2404 |
| I-2405 | I-2406 | I-2407 | I-2408 | I-2409 |
| I-2410 | I-2411 | I-2412 | I-2413 | I-2414 |
| I-2417 | I-2418 | I-2419 | I-2420 | I-2421 |
| I-2422 | I-2423 | I-2424 | I-2425 | I-2426 |
| I-2427 | I-2428 | I-2429 | I-2430 | I-2431 |
| I-2432 | I-2433 | I-2434 | I-2435 | I-2436 |
| I-2437 | I-2438 | I-2439 | I-2440 | I-2441 |
| I-2442 | I-2443 | I-2444 | I-2445 | I-2446 |
| I-2447 | I-2448 | I-2449 | I-2450 | I-2451 |
| I-2452 | I-2453 | I-2454 | I-2455 | I-2456 |
| I-2457 | I-2458 | I-2459 | I-2460 | I-2461 |
| I-2462 | I-2463 | I-2464 | I-2465 | I-2466 |
| I-2467 | I-2468 | I-2469 | I-2470 | I-2471 |
| I-2472 | I-2473 | I-2474 | I-2475 | I-2476 |
| I-2477 | I-2478 | I-2479 | I-2480 | I-2481 |
| I-2482 | I-2483 | I-2484 | I-2485 | I-2486 |
| I-2487 | I-2488 | I-2489 | I-2490 | I-2491 |
| I-2492 | I-2493 | I-2494 | I-2495 | I-2496 |
| I-2497 | I-2498 | I-2499 | I-2500 | I-2501 |
| I-2502 | I-2503 | I-2504 | I-2505 | I-2506 |
| I-2507 | I-2508 | I-2509 | I-2510 | I-2511 |
| I-2512 | I-2513 | I-2514 | I-2515 | I-2516 |
| I-2517 | I-2518 | I-2519 | I-2520 | I-2521 |
| I-2522 | I-2523 | I-2524 | I-2525 | I-2526 |
| I-2527 | I-2528 | I-2529 | I-2530 | I-2531 |
| I-2532 | I-2533 | I-2534 | I-2535 | I-2536 |
| I-2537 | I-2538 | I-2539 | I-2540 | I-2541 |
| I-2542 | I-2543 | I-2544 | I-2545 | I-2546 |
| I-2547 | I-2548 | I-2549 | I-2550 | I-2551 |
| I-2552 | I-2553 | I-2554 | I-2555 | I-2556 |
| I-2557 | I-2558 | I-2559 | I-2560 | I-2561 |
| I-2562 | I-2563 | I-2564 | I-2565 | I-2566 |
| I-2567 | I-2568 | I-2569 | I-2570 | I-2571 |
| I-2572 | I-2573 | I-2574 | I-2575 | I-2576 |
| I-2577 | I-2578 | I-2579 | I-2580 | I-2581 |
| I-2582 | I-2583 | I-2584 | I-2585 | I-2586 |
| I-2587 | I-2588 | I-2589 | I-2590 | I-2591 |
| I-2592 | I-2593 | I-2594 | I-2595 | I-2596 |
| I-2597 | I-2598 | I-2599 | I-2600 | I-2601 |
| I-2602 | I-2603 | I-2604 | I-2605 | I-2606 |
| I-2607 | I-2608 | I-2609 | I-2610 | I-2611 |
| I-2612 | I-2613 | I-2614 | I-2615 | I-2616 |
| I-2617 | I-2618 | I-2619 | I-2620 | I-2621 |
| I-2622 | I-2623 | I-2624 | I-2625 | I-2626 |
| I-2627 | I-2628 | I-2629 | I-2630 | I-2631 |
| I-2632 | I-2633 | I-2634 | I-2635 | I-2636 |
| I-2637 | I-2638 | I-2639 | I-2640 | I-2641 |
| I-2642 | I-2643 | I-2644 | I-2645 | I-2646 |
| I-2647 | I-2648 | I-2649 | I-2650 | I-2651 |
| I-2652 | I-2653 | I-2654 | I-2655 | I-2656 |
| I-2657 | I-2658 | I-2659 | I-2660 | I-2661 |
| I-2662 | I-2663 | I-2664 | I-2665 | I-2666 |
| I-2667 | I-2668 | I-2669 | I-2670 | I-2671 |
| I-2672 | I-2673 | I-2674 | I-2675 | I-2676 |
| I-2677 | I-2678 | I-2679 | I-2680 | I-2682 |
| I-2684 | I-2685 | I-2686 | I-2687 | I-2688 |
| I-2689 | I-2690 | I-2691 | I-2692 | I-2693 |
| I-2696 | I-2697 | I-2698 | I-2699 | I-2700 |
| I-2708 | I-2709 | I-2710 | I-2711 | I-2712 |
| I-2713 | I-2714 | I-2715 | I-2716 | I-2717 |
| I-2718 | I-2719 | I-2720 | I-2721 | I-2722 |
| I-2723 | I-2724 | I-2725 | I-2726 | I-2727 |
| I-2728 | I-2729 | I-2730 | I-2731 | I-2732 |
| I-2733 | I-2734 | I-2735 | I-2736 | I-2737 |
| I-2738 | I-2739 | I-2740 | I-2741 | I-2742 |
| I-2743 | I-2744 | I-2745 | I-2746 | I-2747 |
| I-2752 | I-2753 | I-2754 | I-2755 | I-2757 |
| I-2758 | I-2759 | I-2760 | I-2761 | I-2762 |
| I-2763 | I-2770 | I-2771 | I-2772 | I-2773 |
| I-2774 | I-2775 | I-2777 | I-2778 | I-2779 |
| I-2780 | I-2781 | I-2782 | I-2783 | I-2784 |
| I-2785 | I-2786 | I-2787 | I-2788 | I-2789 |
| I-2790 | I-2791 | I-2792 | I-2793 | I-2794 |
| I-2795 | I-2796 | I-2797 | I-2798 | I-2799 |
| I-2801 | I-2802 | I-2803 | I-2804 | I-2805 |
| I-2808 | I-2809 | I-2810 | I-281 | I-2812 |
| I-2813 | I-2814 | I-2815 | I-2816 | I-2817 |
| I-2818 | I-2823 | I-2824 | I-2825 | I-2826 |
| I-2827 | I-2828 | I-2829 | I-2830 | I-2831 |
| I-2832 | I-2833 | I-2834 | I-2835 | I-2836 |
| I-2837 | I-2838 | I-2839 | I-2840 | I-2843 |
| I-2844 | I-2845 | I-2846 | I-2857 | I-2858 |
| I-2859 | I-2860 | I-2861 | I-2862 | I-2863 |
| I-2864 | I-2865 | I-2866 | I-2867 | I-2868 |
| I-2869 | I-2870 | I-2871 | I-2872 | I-2873 |
| I-2874 | I-2875 | I-2876 | I-2877 | I-2878 |
| I-2879 | I-2880 | I-2881 | I-2884 | I-2885 |
| I-2886 | I-2887 | I-2888 | I-2889 | I-2890 |
| I-2891 | I-2892 | I-2893 | I-2894 | I-2895 |
| I-2902 | I-2903 | I-2904 | I-2905 | I-2906 |
| I-2915 | I-2916 | I-2917 | I-2918 | I-2919 |
| I-2920 | I-2921 | I-2922 | I-2923 | I-2925 |
| I-2926 | I-2927 | I-2928 | I-2929 | I-2942 |
| I-2943 | I-2944 | I-2945 | I-2946 | I-2947 |
| I-2948 | I-2949 | I-2950 | I-2951 | I-2952 |
| I-2953 | I-2954 | I-2955 | I-2956 | I-2957 |
| I-2958 | I-2959 | I-2960 | I-2961 | I-2962 |
| I-2963 | I-2964 | I-2965 | I-2966 | I-2967 |
| I-2973 | I-2974 | I-2975 | I-2976 | I-2977 |
| I-2978 | I-2979 | I-2980 | I-2981 | I-2982 |
| I-2983 | I-2984 | I-2985 | I-2989 | I-2990 |
| I-2991 | I-2996 | I-2997 | I-2998 | I-2999 |
| I-3000 | I-3001 | I-3002 | I-3003 | I-3004 |
| I-3005 | I-3006 | I-3007 | I-3008 | I-3009 |
| I-3010 | I-3011 | I-3012 | I-3013 | I-3014 |
| I-3015 | I-3016 | I-3017 | I-3018 | I-3019 |
| I-3020 | I-3021 | I-3022 | I-3023 | I-3024 |
| I-3025 | I-3026 | I-3027 | I-3028 | I-3029 |
| I-3044 | I-3045 | I-3046 | I-3047 | I-3048 |
| I-3049 | I-3050 | I-3051 | I-3052 | I-3053 |
| I-3054 | I-3055 | I-3056 | I-3057 | I-3058 |
| I-3059 | I-3060 | I-3061 | I-3062 | I-3063 |
| I-3064 | I-3065 | I-3067 | I-3068 | I-3069 |
| I-3070 | I-3071 | I-3073 | I-3081 | I-3082 |
| I-3083 | I-3084 | I-3085 | I-3086 | I-3087 |
| I-3088 | I-3089 | I-3091 | I-3092 | I-3093 |
| I-3094 | I-3095 | I-3096 | I-3098 | I-3099 |
| I-3100 | I-3101 | I-3102 | I-3103 | I-3104 |
| I-3105 | I-3106 | I-3109 | I-3110 | I-3111 |
| I-3112 | I-3113 | I-3115 | I-3118 | I-3119 |
| I-3120 | I-3121 | I-3122 | I-3123 | I-3124 |
| I-3125 | I-3126 | I-3127 | I-3128 | I-3129 |
| I-3130 | I-3131 | I-3132 | I-3134 | I-3137 |
| I-3138 | I-3139 | I-3140 | I-3141 | I-3144 |
| I-3145 | I-3146 | I-3147 | I-3148 | I-3149 |
| I-3150 | I-3151 | I-3152 | I-3153 | I-3154 |
| I-3156 | I-3157 | I-3160 | I-3161 | I-3164 |
| I-3166 | I-3167 | I-3168 | I-3169 | I-3170 |
| I-3171 | I-3172 | I-3173 | I-3174 | I-3175 |
| I-3176 | I-3177 | I-3178 | I-3179 | I-3180 |
| I-3181 | I-3182 | I-3183 | I-3184 | I-3185 |
| I-3186 | I-3187 | I-3188 | I-3189 | I-3190 |
| I-3191 | I-3192 | I-3193 | I-3194 | I-3195 |
| I-3202 | I-3203 | I-3204 | I-3212 | I-3213 |
| I-3214 | I-3215 | I-3216 | I-3217 | I-3218 |
| I-3219 | I-3220 | I-3221 | I-3222 | I-3223 |
| I-3224 | I-3225 | I-3226 | I-3227 | I-3228 |
| I-3232 | I-3233 | I-3234 | I-3235 | I-3238 |
| I-3239 | I-3240 | I-3241 | I-3242 | I-3243 |
| I-3244 | I-3245 | I-3246 | I-3247 | I-3248 |
| I-3249 | I-3250 | I-3251 | I-3252 | I-3253 |
| I-3254 | I-3255 | I-3256 | I-3257 | I-3258 |
| I-3259 | I-3260 | I-3261 | I-3262 | I-3263 |
| I-3264 | I-3265 | I-3266 | I-3267 | I-3268 |
| I-3269 | I-3273 | I-3274 | I-3276 | I-3277 |
| I-3278 | I-3279 | I-3280 | I-3281 | I-3282 |
| I-3283 | I-3284 | I-3285 | I-3286 | I-3287 |
| I-3288 | I-3289 | I-3290 | I-3291 | I-3292 |
| I-3293 | I-3294 | I-3297 | I-3298 | I-3316 |
| I-3317 | I-3318 | I-3319 | I-3320 | I-3321 |
| I-3324 | I-3325 | I-3326 | I-3327 | I-3328 |
| I-3329 | I-3331 | I-3332 | I-3333 | I-3334 |
| I-3335 | I-3336 | I-3337 | I-3338 | I-3339 |
| I-3340 | I-3341 | I-3342 | I-3350 | I-3351 |
| I-3352 | I-3353 | I-3354 | I-3355 | I-3356 |
| I-3357 | I-3358 | I-3359 | I-3367 | I-3373 |
| I-3374 | I-3375 | I-3376 | I-3377 | I-3378 |
| I-3379 | I-3380 | I-3381 | I-3382 | I-3383 |
| I-3384 | I-3385 | I-3386 | I-3387 | I-3388 |
| I-3389 | I-3390 | I-3391 | I-3392 | I-3393 |
| I-3394 | I-3395 | I-3396 | I-3398 | I-3401 |
| I-3402 | I-3403 | I-3404 | I-3405 | I-3406 |
| I-3408 | I-3409 | I-3410 | I-3411 | I-3414 |
| I-3415 | I-3416 | I-3417 | I-3423 | I-3425 |
| I-3426 | I-3427 | I-3428 | I-3429 | I-3430 |
| I-3431 | I-3432 | I-3433 | I-3434 | I-3435 |
| I-3436 | I-3437 | I-3438 | I-3441 | I-3442 |
| I-3443 | I-3444 | I-3449 | I-3450 | I-3451 |
| I-3479 | I-3487 | I-3488 | I-3489 | I-3490 |
| I-3491 | I-3492 | I-3493 | I-3494 | I-3495 |
| I-3496 | I-3497 | I-3498 | I-3499 | I-3500 |
| I-3501 | I-3502 | I-3503 | I-3504 | I-3505 |
| I-3506 | I-3507 | I-3508 | I-3509 | I-3510 |
| I-3511 | I-3512 | I-3513 | I-3514 | I-3515 |
| I-3516 | I-3518 | I-3519 | I-3521 | I-3522 |
| I-3526 | I-3527 | I-3528 | I-3529 | I-3530 |
| I-3531 | I-3532 | I-3533 | I-3535 | I-3536 |
| I-3537 | I-3538 | I-3539 | I-3540 | I-3541 |
| I-3544 | I-3545 | I-3546 | I-3547 | I-3548 |
| I-3586 | I-3587 | I-3588 | I-3589 | I-3590 |
| I-3591 | I-3592 | I-3593 | I-3595 | I-3596 |
| I-3597 | I-3598 | I-3599 | I-3600 | I-3605 |
| I-3606 | I-3607 | I-3608 | I-3609 | I-3610 |
| I-3615 | I-3616 | I-3617 | I-3618 | I-3619 |
| I-3620 | I-3621 | I-3622 | I-3624 | I-3626 |
| I-3627 | I-3629 | I-3630 | I-3634 | I-3635 |
| I-3636 | I-3637 | I-3638 | I-3639 | I-3640 |
| I-3641 | I-3642 | I-3643 | I-3644 | I-3645 |
| I-3646 | I-3647 | I-3648 | I-3649 | I-3650 |
| I-3671 | I-3673 | I-3678 | I-3679 | I-3680 |
| I-3682 | I-3683 | I-3688 | I-3689 | I-3691 |
| I-3692 | I-3693 | I-3694 | I-3695 | I-3697 |
| I-3698 | I-3699 | I-3700 | I-3701 | I-3702 |
| I-3703 | I-3704 | I-3705 | I-3706 | I-3707 |
| I-3709 | I-3710 | I-3712 | I-3713 | I-3717 |
| I-3718 | I-3721 | I-3723 | I-3724 | I-3727 |
| I-3728 | I-3729 | I-3730 | I-3731 | I-3732 |
| I-3733 | I-3734 | I-3735 | I-3736 | I-3737 |
| I-3738 | I-3739 | I-3740 | | |

### Example 33

### rac-(1R,3R)-3′-oxospiro[cyclopentane-1,1′-isoindoline]-3-carboxylic acid and rac-(1R,3S)-3′-oxospiro[cyclopentane-1,1′-isoindoline]-3-carboxylic acid

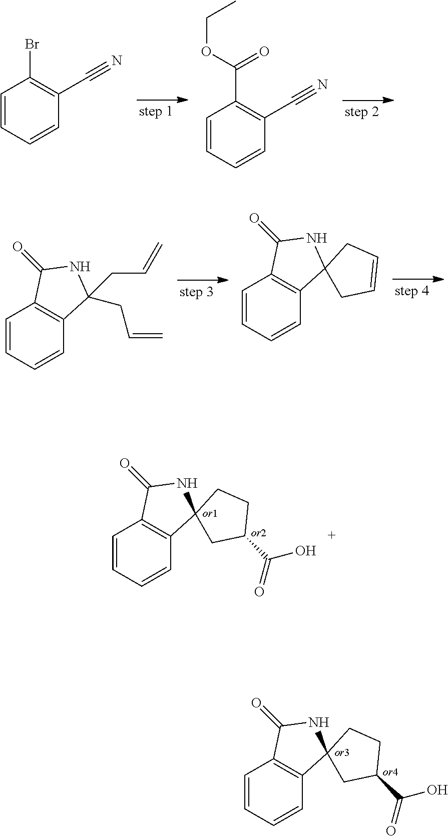 [SMILES: N#Cc1ccccc1Br ; CCOC(=O)c1ccccc1C#N ; C=CCC1(CC=C)NC(=O)c2ccccc21 ; O=C1NC2(CC=CC2)c2ccccc21 ; O=C1N[C@]2(CC[C@H](C(=O)O)C2)c2ccccc21 ; O=C1N[C@]2(CC[C@@H](C(=O)O)C2)c2ccccc21]

### Step 1. Synthesis of ethyl 2-cyanobenzoate

A 50 mL flame dried flask was charged with 2-bromobenzonitrile (1.00 g, 5.49 mmol). Dry THF (50 mL) was added under a N₂ atmosphere and the reaction media was cooled down to −78° C. n-butyllithium (2.64 mL, 2.5 molar in THF, 6.59 mmol) was added dropwise and the reaction was stirred for 15 minutes. Ethyl carbonocyanidate (651 μL, 6.59 mmol) was added and the reaction was slowly warmed up to room temperature. After 1 hour the reaction was quenched with saturated NH₄Cl solution. The aqueous layer was extracted twice with EtOAc. The organic layers were combined and washed once with water, then dried over Na₂SO₄ and concentrated under reduced pressure. The material was purified by normal phase column chromatography (hexanes:EtOAc 100:0 to 80:20) to give ethyl 2-cyanobenzoate (396 mg) as a viscous colorless oil. 1H NMR: (400 MHz, CDCl₃) δ 8.12-8.08 (m, 1H), 7.76 (dd, J=7.0, 1.6 Hz, 1H), 7.67-7.59 (m, 2H), 4.42 (q, J=7.1 Hz, 2H), 1.40 (t, J=7.1 Hz, 3H).

### Step 2. Synthesis of 3,3-diallylisoindolin-1-one

To a suspension of zinc dust (224 mg, 3.42 mmol) in THF (5 mL) were successively added ethyl 2-cyanobenzoate (200 mg, 1.14 mmol) in THF (0.5 mL) and allyl bromide (294 μL, 3.42 mmol). The solution was heated to reflux and cooled down to room temperature after 15 min of heating. HCl (1N, 5 mL) was added to the reaction and the aqueous layer was extracted with EtOAc (2×5 mL). The combined organic layers were washed with 2 N NaOH and brine, dried over anhydrous Na₂SO₄ and filtered. The solvent was removed in vacuo to afford 3,3-diallylisoindolin-1-one (240 mg, crude) as a yellow viscous oil. LCMS RT 1.39 min, [M+H]⁺ 214.1, LCMS method L.

### Step 3. Synthesis of spiro[cyclopentane-1,1′-isoindolin]-3-en-3′-one

A 150 mL flame-dried flask was charged with 3,3-diallylisoindolin-1-one (240 mg, 1.13 mmol). Dry toluene (40 mL) was added under a N₂ atmosphere and the reaction was heated to 70° C. Benzylidene(dichloro)(1,3-dimesityl-2-imidazolidinylidene)ruthenium -tricyclohexylphosphine (1:1) (47.8 mg, 56.3 μmol) was added and the reaction was kept at this temperature for 90 min. After cooling to room temperature, the reaction media was concentrated under reduced pressure and the crude material was purified using normal phase column chromatography (DCM:EtOAc 100:0 to 50:50) to give spiro[cyclopentane-1,1′-isoindolin]-3-en-3′-one (98 mg) as a colorless oil. ¹H NMR (400 MHz, CDCl₃) δ 7.79 (d, J=7.5 Hz, 1H), 7.60-7.51 (m, 2H), 7.50-7.40 (m, 2H), 5.90 (d, J=8.2 Hz, 1H), 5.88 (d, J=8.3 Hz, 1H), 2.89 (d, J=15.8 Hz, 1H), 2.89 (d, J=15.8 Hz, 1H), 2.77 (d, J=15.7 Hz, 1H). LCMS RT 1.23 min, [M+H]⁺ 186.1, LCMS method L.

### Step 4. Synthesis of rac-(1R,3R)-3′-oxospiro[cyclopentane-1,1′-isoindoline]-3-carboxylic acid and rac-(1R,3S)-3′-oxospiro[cyclopentane-1,1′-isoindoline]-3-carboxylic acid

To a flame dried 10 mL flask was added palladium diacetate (6.06 mg, 27.0 μmol) and 4,5-bis(diphenylphosphino)-9,9-dimethyl xanthene (15.6 mg, 27.0 mol). Dry PhMe (0.2 mL) was added under a N₂ atmosphere followed by spiro[cyclopentane-1,1′-isoindolin]-3-en-3′-one (100 mg, 540 μmol), formic acid (41.2 μL, 1.08 mmol), and acetic anhydride (10.2 μL, 108 μmol) successively via syringe. The vial was purged with N₂ and tightly sealed with a septum cap. The reaction mixture was stirred at 70° C. for 24 hours. The reaction media was cooled down to room temperature and diluted with DCM and HCl (1 N). The aqueous layer was extracted with DCM 3 times. The organic layers were dried over Na₂SO₄ and concentrated under reduced pressure. The crude material was diluted in DMF and purified on a 30 g C18 column (mobile phase A: 10 mM ammonium formate in water, mobile phase B: acetonitrile, gradient: A:B 95:5 to 70:30) to give rac-(1R,3R)-3′-oxospiro[cyclopentane-1,1′-isoindoline]-3-carboxylic acid and rac-(1R,3S)-3′-oxospiro[cyclopentane-1,1′-isoindoline]-3-carboxylic acid. Isomer 1: 28 mg, LCMS RT 1.04 min, [M+H]⁺ 232.0, LCMS method L. Isomer 2: 29 mg, LCMS RT 1.09 min, [M+H]⁺ 232.0, LCMS method L.

Additional compounds prepared according to the methods of Example 33 are listed in Table 3 below. Corresponding ¹H NMR and mass spectrometry characterization for these compounds are described in Table 1. Certain compounds in Table 3 below were prepared with other compounds whose preparation is described further below in the Examples.

TABLE 3
Additional exemplary compounds

| | | | | |
|---|---|---|---|---|
| I-1898 | I-1905 | I-1982 | I-1983 | I-2022 |
| I-2023 | I-2024 | I-2025 | I-2026 | I-2027 |
| I-2028 | I-2029 | I-2120 | I-2121 | I-2122 |
| I-2123 | I-2124 | I-2125 | I-2126 | I-2127 |

### Example 34

### (1S,3R,4S)—N—((S)-(3-chloro-2,6-difluorophenyl)(4-fluorobicyclo[2.2.1]heptan-1-yl)methyl)-3-hydroxy-4-(pyridazin-3-ylamino)cyclopentane-1-carboxamide and (1R,3R,4S)—N—((S)-(3-chloro-2,6-difluorophenyl)(4-fluorobicyclo[2.2.1]heptan-1-yl)methyl)-3-hydroxy-4-(pyridazin-3-ylamino)cyclopentane-1-carboxamide

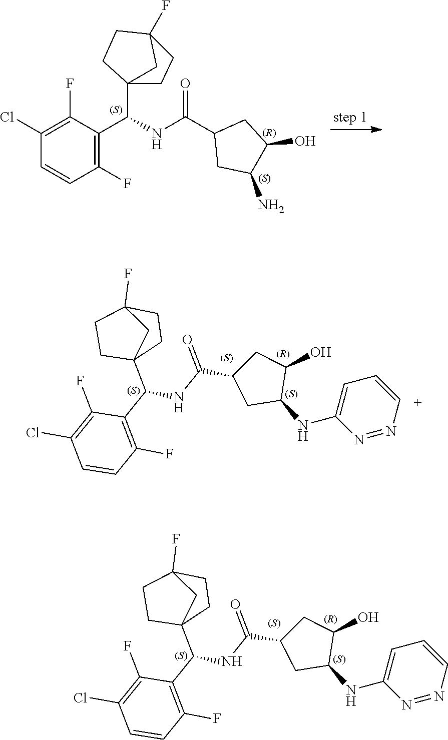

### Step 1. Synthesis of (1S,3R,4S)—N—((S)-(3-chloro-2,6-difluorophenyl)(4-fluorobicyclo[2.2.1]heptan-1-yl)methyl)-3-hydroxy-4-(pyridazin-3-ylamino)cyclopentane-1-carboxamide and (1R,3R,4S)—N—((S)-(3-chloro-2,6-difluorophenyl)(4-fluorobicyclo[2.2.1]heptan-1-yl)methyl)-3-hydroxy-4-(pyridazin-3-ylamino)cyclopentane-1-carboxamide

A mixture of (3S,4R)-3-amino-N—((S)-(3-chloro-2,6-difluorophenyl)(4-fluorobicyclo[2.2.1]heptan-1-yl)methyl)-4-hydroxycyclopentane-1-carboxamide (100 mg, 240 μmol), 3-bromopyridazine (45.8 mg, 288 μmol), Cs₂CO₃ (235 mg, 720 μmol) and Pd-PEPPSI-IHept-Cl (46.7 mg, 48.0 μmol) in 1,4-dioxane (5 mL) was stirred for 6 hours at 90° C. under a N₂ atmosphere. The resulting crude material was purified by preparative HPLC (column: XBridge Prep OBD C18 Column, 30*150 mm, 5 μm; mobile phase A: water (10 mmol/L NH4HCO₃+0.05% NH₄OH), mobile phase B: acetonitrile; flow rate: 60 mL/min; gradient: 42% B to 62% B in 8 min; wavelength: 220 nm; RT (min): 8.52) to give an off-white solid (50 mg, 42%). The solid was further purified by chiral HPLC (column: CHIRALPAKIG3; mobile phase A: hexane (0.2% diethylamine); mobile phase B: (EtOH:DCM 1:1); flow rate: 1 mL/min; gradient: isocratic; injection volume: 3 mL) to give (1S,3R,4S)—N—((S)-(3-chloro-2,6-difluorophenyl)(4-fluorobicyclo[2.2.1]heptan-1-yl)methyl)-3-hydroxy-4-(pyridazin-3-ylamino)cyclopentane-1-carboxamide and (1R,3R,4S)—N—((S)-(3-chloro-2,6-difluorophenyl)(4-fluorobicyclo[2.2.1]heptan-1-yl)methyl)-3-hydroxy-4-(pyridazin-3-ylamino)cyclopentane-1-carboxamide, both as an amorphous off-white solid. Peak 1:10.9 mg (22.0 μmol). ¹H NMR (400 MHz, DMSO-d6) δ 8.37 (dd, J=4.3, 1.4 Hz, 1H), 8.24 (d, J=8.2 Hz, 1H), 7.56 (td, J=8.7, 5.4 Hz, 1H), 7.22-7.10 (m, 2H), 6.87 (dd, J=9.0, 1.5 Hz, 1H), 6.47 (d, J=7.0 Hz, 1H), 5.27 (d, J=8.2 Hz, 1H), 4.88 (d, J=3.7 Hz, 1H), 4.17 (d, J=5.5 Hz, 2H), 3.15 (qd, J=8.6, 6.0 Hz, 1H), 1.93-1.85 (m, 2H), 1.84-1.74 (m, 6H), 1.71 (d, J=12.3 Hz, 3H), 1.60 (d, J=8.4 Hz, 1H), 1.46 (d, J=9.1 Hz, 2H). LCMS RT 0.94 min, [M+H]⁺ 495, LCMS method D; Peak 2: 20.0 mg (40.4 μmol)¹H NMR (400 MHz, DMSO-d6) δ 8.38 (dd, J=4.4, 1.4 Hz, 1H), 8.24 (d, J=8.2 Hz, 1H), 7.57 (td, J=8.7, 5.4 Hz, 1H), 7.30-7.04 (m, 2H), 6.90 (dd, J=9.1, 1.4 Hz, 1H), 6.50 (d, J=7.5 Hz, 1H), 5.29 (d, J=8.2 Hz, 1H), 4.88 (d, J=3.6 Hz, 1H), 4.28-4.10 (m, 2H), 3.14 (dd, J=12.7, 7.9 Hz, 1H), 2.02 (ddd, J=12.7, 8.1, 4.8 Hz, 1H), 1.90-1.78 (m, 4H), 1.77-1.65 (m, 6H), 1.60 (d, J=8.4 Hz, 1H), 1.46 (d, J=8.6 Hz, 2H). LCMS RT 0.94 min, [M+H]⁺ 495, LCMS method D.

Additional compounds prepared according to the methods of Example 34 are listed in Table 4 below. Corresponding ¹H NMR and mass spectrometry characterization for these compounds are described in Table 1. Certain compounds in Table 4 below were prepared with other compounds whose preparation is described further below in the Examples.

TABLE 4
Additional exemplary compounds

| | | | | |
|---|---|---|---|---|
| I-2015 | I-2016 | I-2094 | I-2108 | I-2111 |
| I-2128 | I-2130 | I-2132 | I-2135 | I-2156 |
| I-2159 | I-2160 | I-2221 | I-2312 | I-2315 |
| I-2321 | I-2324 | I-2343 | I-2800 | I-2806 |
| I-2807 | I-2841 | I-2842 | I-2924 | I-3439 |
| I-3440 | I-3542 | I-3543 | I-3623 | I-3625 |
| I-3628 | I-3674 | I-3675 | I-3690 | I-3696 |
| I-3711 | I-3719 | I-3725 | I-3726 | |

### Example 35

### (S)-1-(1-acetyl-4-methylpiperidin-4-yl)-3-((3-chlorophenyl)(cyclopentyl)methyl)urea

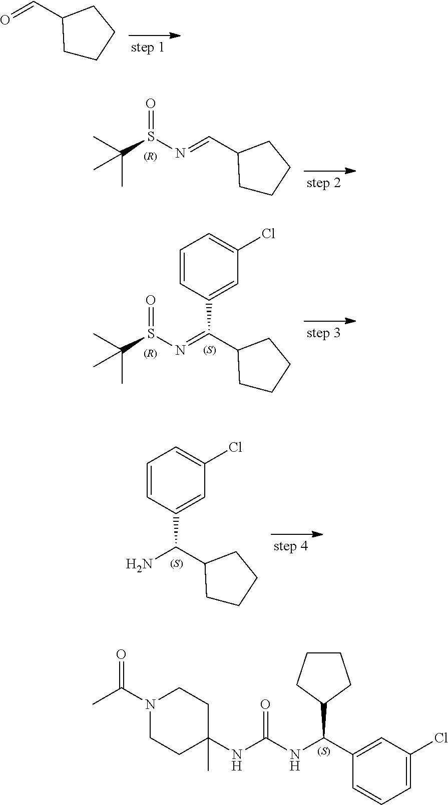

### Step 1. Synthesis of (R)—N-(cyclopentylmethylene)-2-methylpropane-2-sulfinamide

To a solution of cyclopentanecarbaldehyde (112 g, 1.15 mol) and (R)-2-methylpropane-2-sulfinamide (167 g, 1.38 mol) in THF (560 mL) was added Ti(OⁱPr)₄ (651 g, 2.29 mol) under a N₂ atmosphere at 25° C. The mixture was heated to 75° C. and stirred for 2 hours. After cooling to room temperature, to the mixture was added brine (3.00 L). The suspension was filtered. The filter cake was washed with ethyl acetate (5.00 L*2). The organic phase in the filtrate was separated and the aqueous phase was extracted with ethyl acetate (3.00 L). The combined organic phase was washed with brine (3.0 L), dried over Na₂SO₄, filtered and concentrated. The residue was purified by column chromatography (silica gel, petroleum ether:ethyl acetate 1:0 to 10:1) to give (R)—N-(cyclopentylmethylene)-2-methylpropane-2-sulfinamide (357 g, 1.77 mol) as a yellow oil. ¹H NMR (400 MHz CDCl₃)

δ 8.00 (d, J=5.6 Hz, 1H), 2.98-2.94 (m, 1H), 1.94-1.83 (m, 2H), 1.77-1.62 (m, 6H), 1.19 (s, 9H).

### Step 2. Synthesis of (R)—N—((S)-(3-chlorophenyl)(cyclopentyl)methyl)-2-methylpropane-2-sulfinamide

Two batches were executed. To a solution of (R)—N-(cyclopentylmethylene)-2-methylpropane-2-sulfinamide (160 g, 795 mmol) and 1-bromo-3-chlorobenzene (140 mL, 1.19 mol) in THF (800 mL) was added n-BuLi (2.50 M in THF, 477 mL) dropwise at −60˜−70° C. under N₂. The reaction was stirred between -70 and −60° C. for 2 hours. Two batches of mixture were combined. The mixture was poured into saturated NH₄Cl solution (5.0 L) and extracted with ethyl acetate (2.00 L*3). Then the combined organic phase was washed with brine (2.00 L), dried over Na₂SO₄, filtered and concentrated to give the crude product (R)—N—((S)-(3-chlorophenyl)(cyclopentyl)methyl)-2-methylpropane-2-sulfinamide as a yellow oil (563 g), which was used in the next step without purification.

### Step 3. Synthesis of (S)-(3-chlorophenyl)(cyclopentyl)methanamine

Two batches were carried out in parallel. To a solution of (R)—N—((S)-(3-chlorophenyl)(cyclopentyl)methyl)-2-methylpropane-2-sulfinamide (264 g, 757 mmol) in ethyl acetate (2.60 L) was added HCl (4 N in EtOAc, 473 mL) at 25° C. The mixture was stirred at 25° C. for 1 hour. After 1 hour of stirring, a large amount of white solid was formed. Two batches of reaction mixture were combined. The suspension was concentrated to 4.0 L. The suspension was filtered and the filter cake was washed with ethyl acetate (200 mL*2). Then the filter cake was partitioned between ethyl acetate (2.00 L) and saturated NaHCO₃ solution (2.50 L). The suspension was stirred for 10 minutes until the solid disappeared. The organic phase was separated and the aqueous phase was extracted with ethyl acetate (1.00 L*2). The combined organic phase was washed with brine (2.00 L), dried over Na₂SO₄, filtered and concentrated to give the crude product (S)-(3-chlorophenyl)(cyclopentyl)methanamine (220 g) as a yellow oil, which was used in the next step without purification.

### Step 4. Synthesis of (S)-1-(1-acetyl-4-methylpiperidin-4-yl)-3-((3-chlorophenyl)(cyclopentyl)methyl)urea

(3-chlorophenyl)(cyclopentyl)methanamine (100 mg, 477 μmol) was dissolved in DCM (5 mL). The solution was cooled to 0° C. CDI (92.8 mg, 572 μmol) was added, followed by DMAP (5.83 mg, 47.7 μmol). The solution was stirred at 0° C. for 1 hour. 1-(4-amino-4-methylpiperidin-1-yl)ethan-1-one hydrochloride (91.9 mg, 477 μmol) and triethylamine (199 μL, 1.43 mmol) were added, and the solution was stirred at 40° C. for 1.5 h, and then at room temperature overnight. It was then heated at 40° C. for 4.5 h, concentrated and purified by HPLC to give the product 1-(1-acetyl-4-methylpiperidin-4-yl)-3-((3-chlorophenyl)(cyclopentyl)methyl)urea (53.1 mg, 135 μmol) as a colorless solid. LCMS: RT 1.410 min, [M+H]392.46, LCMS method I.

### Example 36

### (1R,3R)—N—((R)-(2,3-dichloro-6-fluorophenyl)(1-methylcyclopentyl)methyl)-3-(3-methylureido)cyclopentane-1-carboxamide

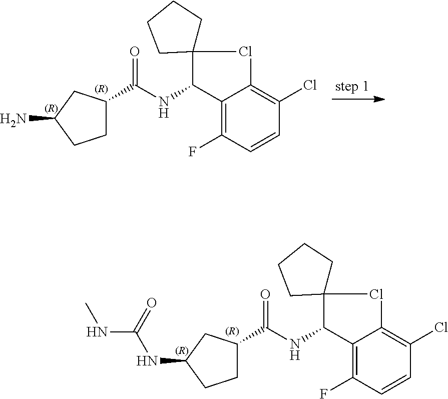

### Step 1. Synthesis of (1R,3R)—N—((R)-(2,3-dichloro-6-fluorophenyl)(1-methylcyclopentyl)methyl)-3-(3-methylureido)cyclopentane-1-carboxamide

A mixture of (1R,3R)-3-amino-N—((R)-(2,3-dichloro-6-fluorophenyl)(1-methylcyclopentyl)methyl)cyclopentane-1-carboxamide (50 mg, 0.13 mmol), N-methyl-1H-imidazole-1-carboxamide (16 mg, 0.13 mmol) and TEA (26 mg, 0.26 mmol) in CH₃CN (1 mL) was stirred for 2 h at 25° C. The reaction was quenched with MeOH (1 mL) and concentrated. The resulting crude material was purified by preparative HPLC (column: XBridge Prep OBD C18 Column, 30*150 mm, 5 μm; mobile phase A: water (10 mM NH₄HCO₃), mobile phase B: acetonitrile; flow rate: 60 mL/min; gradient: 35% B to 65% B in 8 min, then 65% B; wavelength: 220 nm; RT1 (min): 7.53) to give (1R,3R)—N—((R)-(2,3-dichloro-6-fluorophenyl)(1-methylcyclopentyl) methyl)-3-(3-methylureido)cyclopentane-1-carboxamide (17.4 mg, 39.2 μmol) as an off-white solid. ¹H NMR (400 MHz, DMSO-d₆) δ 8.05 (d, J=8.7 Hz, 1H), 7.60 (d, J=9.6 Hz, 1H), 7.25 (t, J=9.8 Hz, 1H), 5.81 (d, J=7.1 Hz, 1H), 5.56 (s, 1H), 5.49 (d, J=8.6 Hz, 1H), 3.88 (q, J=6.5 Hz, 1H), 2.99-2.91 (m, 1H), 2.53 (s, 3H), 1.88-1.70 (m, 3H), 1.68-1.57 (m, 7H), 1.44 (dd, J=12.6, 7.5 Hz, 1H), 1.37 (s, 1H), 1.35-1.24 (m, 2H), 0.96 (d, J=2.8 Hz, 3H). LCMS RT 1.158 min, [M+H]⁺ 444, LCMS method C.

### Example 37

### N-((1S,2R,4S)-4-(((S)-(2,3-dichloro-6-fluorophenyl)(4-fluorobicyclo[2.2.1]heptan-1-yl)methyl)carbamoyl)-2-hydroxycyclopentyl)azetidine-1-carboxamide

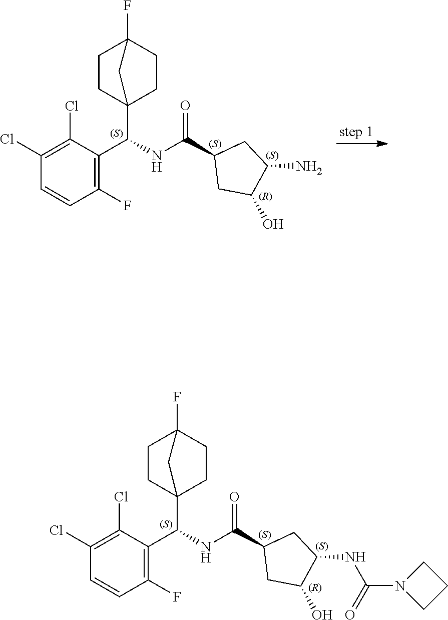

### Step 1. Synthesis of N-((1S,2R,4S)-4-(((S)-(2,3-dichloro-6-fluorophenyl)(4-fluorobicyclo[2.2.1]heptan-1-yl)methyl)carbamoyl)-2-hydroxycyclopentyl)azetidine-1-carboxamide

(1S,3S,4R)-3-amino-N—((S)-(2,3-dichloro-6-fluorophenyl)(4-fluorobicyclo[2.2.1]heptan-1-yl)methyl)-4-hydroxycyclopentane-1-carboxamide was synthesized similarly as example 5. To a stirred solution of azetidine (13 mg, 231 μmol) and TEA (70 mg, 692 μmol) in CH₂Cl₂ (3 mL) was added triphosgene (20 mg, 0.30 Eq, 69.2 μmol) dropwise at 0° C. under a nitrogen atmosphere. The resulting mixture was stirred for 1 hour at 30° C. under nitrogen. To the above mixture was added (1S,3S,4R)-3-amino-N—((S)-(2,3-dichloro-6-fluorophenyl)(4-fluorobicyclo[2.2.1]heptan-1-yl)methyl)-4-hydroxycyclopentane-1-carboxamide (100 mg, 231 μmol) at room temperature. The resulting mixture was stirred for 1 hour at room temperature. The resulting mixture was purified by reversed-phase flash chromatography (column: XBridge Prep OBD C18 Column, 30*150 mm, 10 μm; mobile phase A: water (10 mM NH₄HCO₃+0.05% NH₄OH), mobile phase B: Acetonitrile; flow rate: 60 mL/min; gradient: 32% B to 49% B in 8 minutes; wavelength: 254 nm/220 nm; RT (min): 9.48) to give N-((1S,2R,4S)-4-(((S)-(2,3-dichloro-6-fluorophenyl)(4-fluorobicyclo[2.2.1]heptan-1-yl)methyl)carbamoyl)-2-hydroxycyclopentyl)azetidine-1-carboxamide (4.3 mg, 8.0 μmol) as a white solid.

LCMS RT 1.389 min, [M+H]⁺ 516.20. LCMS Method F. ¹H NMR (300 MHz, DMSO-d6) δ 8.17 (d, J=8.2 Hz, 1H), 7.62 (dd, J=8.9, 5.1 Hz, 1H), 7.26 (dd, J=10.6, 9.0 Hz, 1H), 5.49 (d, J=7.7 Hz, 2H), 4.77 (d, J=3.5 Hz, 1H), 3.91-3.85 (m, 1H), 3.78 (dd, J=9.9, 5.1 Hz, 4H), 3.12-2.98 (m, 1H), 2.15-2.07 (m, 2H), 1.85-1.49 (m, 15H1). ¹⁹F NMR (282 MHz, DMSO) δ −109.27, −173.53, −173.77.

Additional compounds prepared according to the methods of Examples 35-37 are listed in Table 5 below. Corresponding ¹H NMR and mass spectrometry characterization for these compounds are described in Table 1. Certain compounds in Table 5 below were prepared with other compounds whose preparation is described further below in the Examples.

TABLE 5
Additional Exemplary Compounds

| | | | | |
|---|---|---|---|---|
| I-1 | I-2 | I-4 | I-5 | I-7 |
| I-8 | I-9 | I-10 | I-11 | I-12 |
| I-13 | I-14 | I-15 | I-16 | I-17 |
| I-18 | I-19 | I-20 | I-21 | I-22 |
| I-23 | I-24 | I-25 | I-26 | I-27 |
| I-28 | I-29 | I-30 | I-31 | I-32 |
| I-33 | I-34 | I-35 | I-36 | I-37 |
| I-38 | I-39 | I-40 | I-41 | I-42 |
| I-43 | I-44 | I-45 | I-46 | I-47 |
| I-48 | I-49 | I-50 | I-51 | I-52 |
| I-53 | I-54 | I-55 | I-56 | I-57 |
| I-58 | I-59 | I-60 | I-61 | I-62 |
| I-63 | I-64 | I-65 | I-66 | I-67 |
| I-68 | I-69 | I-70 | I-71 | I-72 |
| I-73 | I-74 | I-75 | I-76 | I-77 |
| I-78 | I-79 | I-80 | I-81 | I-82 |
| I-84 | I-85 | I-86 | I-87 | I-88 |
| I-89 | I-90 | I-91 | I-92 | I-93 |
| I-94 | I-95 | I-96 | I-97 | I-98 |
| I-99 | I-100 | I-101 | I-102 | I-103 |
| I-104 | I-105 | I-106 | I-107 | I-108 |
| I-109 | I-110 | I-111 | I-112 | I-113 |
| I-114 | I-115 | I-116 | I-117 | I-118 |
| I-119 | I-120 | I-121 | I-122 | I-123 |
| I-124 | I-125 | I-126 | I-127 | I-128 |
| I-129 | I-130 | I-131 | I-132 | I-133 |
| I-134 | I-135 | I-136 | I-137 | I-138 |
| I-139 | I-140 | I-141 | I-142 | I-143 |
| I-144 | I-145 | I-146 | I-147 | I-148 |
| I-149 | I-150 | I-151 | I-152 | I-153 |
| I-154 | I-155 | I-156 | I-157 | I-158 |
| I-159 | I-160 | I-161 | I-162 | I-163 |
| I-164 | I-165 | I-166 | I-167 | I-168 |
| I-169 | I-170 | I-171 | I-172 | I-173 |
| I-174 | I-175 | I-176 | I-177 | I-178 |
| I-179 | I-182 | I-184 | I-185 | I-186 |
| I-187 | I-188 | I-189 | I-190 | I-195 |
| I-196 | I-197 | I-204 | I-205 | I-206 |
| I-207 | I-215 | I-219 | I-220 | I-221 |
| I-223 | I-225 | I-230 | I-231 | I-232 |
| I-233 | I-235 | I-254 | I-255 | I-256 |
| I-261 | I-262 | I-264 | I-265 | I-267 |
| I-268 | I-274 | I-275 | I-276 | I-277 |
| I-278 | I-279 | I-280 | I-281 | I-282 |
| I-283 | I-284 | I-285 | I-286 | I-287 |
| I-288 | I-289 | I-290 | I-291 | I-292 |
| I-293 | I-299 | I-300 | I-301 | I-302 |
| I-303 | I-304 | I-305 | I-306 | I-307 |
| I-308 | I-309 | I-310 | I-311 | I-312 |
| I-313 | I-314 | I-315 | I-316 | I-317 |
| I-318 | I-319 | I-320 | I-321 | I-322 |
| I-323 | I-324 | I-326 | I-328 | I-329 |
| I-330 | I-331 | I-332 | I-335 | I-336 |
| I-345 | I-346 | I-347 | I-350 | I-355 |
| I-356 | I-357 | I-359 | I-361 | I-363 |
| I-365 | I-368 | I-374 | I-379 | I-380 |
| I-381 | I-382 | I-383 | I-385 | I-386 |
| I-387 | I-388 | I-389 | I-390 | I-391 |
| I-392 | I-393 | I-394 | I-395 | I-396 |
| I-397 | I-398 | I-399 | I-400 | I-401 |
| I-402 | I-404 | I-405 | I-407 | I-408 |
| I-409 | I-410 | I-412 | I-413 | I-414 |
| I-415 | I-416 | I-417 | I-418 | I-419 |
| I-420 | I-421 | I-422 | I-423 | I-425 |
| I-426 | I-428 | I-431 | I-433 | I-434 |
| I-435 | I-436 | I-443 | I-444 | I-447 |
| I-448 | I-449 | I-450 | I-451 | I-452 |
| I-453 | I-456 | I-457 | I-458 | I-459 |
| I-462 | I-463 | I-464 | I-465 | I-468 |
| I-472 | I-473 | I-474 | I-480 | I-481 |
| I-482 | I-483 | I-486 | I-487 | I-495 |
| I-496 | I-497 | I-499 | I-500 | I-501 |
| I-502 | I-503 | I-516 | I-517 | I-518 |
| I-519 | I-520 | I-523 | I-529 | I-530 |
| I-531 | I-532 | I-533 | I-534 | I-535 |
| I-548 | I-565 | I-584 | I-585 | I-586 |
| I-587 | I-588 | I-589 | I-590 | I-591 |
| I-592 | I-593 | I-594 | I-595 | I-596 |
| I-597 | I-599 | I-601 | I-602 | I-603 |
| I-604 | I-605 | I-617 | I-618 | I-623 |
| I-624 | I-625 | I-626 | I-627 | I-628 |
| I-629 | I-631 | I-632 | I-633 | I-636 |
| I-637 | I-638 | I-646 | I-647 | I-648 |
| I-649 | I-653 | I-654 | I-655 | I-656 |
| I-662 | I-663 | I-664 | I-677 | I-697 |
| I-698 | I-699 | I-705 | I-706 | I-707 |
| I-708 | I-713 | I-714 | I-715 | I-716 |
| I-720 | I-721 | I-722 | I-723 | I-726 |
| I-727 | I-728 | I-729 | I-731 | I-732 |
| I-733 | I-734 | I-735 | I-736 | I-737 |
| I-738 | I-739 | I-740 | I-741 | I-742 |
| I-743 | I-749 | I-750 | I-751 | I-752 |
| I-753 | I-754 | I-759 | I-779 | I-805 |
| I-807 | I-810 | I-813 | I-815 | I-687 |
| I-880 | I-881 | I-884 | I-885 | I-886 |
| I-888 | I-889 | I-890 | I-892 | I-893 |
| I-894 | I-895 | I-896 | I-899 | I-926 |
| I-928 | I-930 | I-933 | I-969 | I-970 |
| I-971 | I-972 | I-991 | I-994 | I-995 |
| I-1006 | I-1008 | I-1016 | I-1112 | I-1113 |
| I-1114 | I-1199 | I-1678 | I-1802 | I-1803 |
| I-1804 | I-1805 | I-1806 | I-1807 | I-1808 |
| I-1809 | I-1810 | I-1811 | I-1812 | I-1813 |
| I-1818 | I-1819 | I-1820 | I-1821 | I-1822 |
| I-1823 | I-1824 | I-1825 | I-1826 | I-1830 |
| I-1831 | I-1832 | I-1894 | I-1915 | I-1919 |
| I-1920 | I-1933 | I-1958 | I-1960 | I-2014 |
| I-2017 | I-2038 | I-2039 | I-2093 | I-2415 |
| I-2416 | I-2756 | I-2764 | I-2765 | I-2776 |
| I-3197 | I-3271 | I-3272 | I-3275 | I-3278 |
| I-3397 | I-3681 | I-3708 | | |

A mixture of (S)—N-((3-chloro-2,6-difluorophenyl)(cyclopentyl)methyl)-1-((2-(trimethylsilyl)ethoxy)methyl)-1H-benzo[d]imidazol-2-amine (150 mg, 305 μmol) in TFA (2 mL) was stirred for 1 hour at 25° C. The reaction mixture was concentrated in vacuo. The resulting crude material was purified by preparative HPLC (column: XBridge Prep OBD C18 Column, 30*150 mm, 5 μm; mobile phase A: water (10 mM NH₄HCO₃), mobile phase B: acetonitrile; flow rate: 60 mL/min; gradient: 40% B to 70% B in 8 min, then 70% B; wavelength: 220 nm; RT (min): 7.83). Concentration in vacuo gave (S)—N-((3-chloro-2,6-difluorophenyl)(cyclopentyl)methyl)-1H-benzo[d]imidazol-2-amine (20 mg, 55 μmol) as an off-white amorphous solid. ¹H NMR (400 MHz, DMSO-d₆) δ 10.31 (s, 1H), 7.53 (td, J=8.6, 5.4 Hz, 1H), 7.20-7.09 (m, 3H), 7.07 (d, J=8.9 Hz, 1H), 6.84 (s, 2H), 5.13 (t, J=9.7 Hz, 1H), 2.51 (s, 1H), 1.96-1.88 (m, 1H), 1.64 (s, 2H), 1.57 (dt, J=15.2, 8.1 Hz, 2H), 1.44 (td, J=12.5, 6.6 Hz, 2H), 1.17-1.09 (m, 1H). LCMS RT 0.815 min, [M+H]⁺ 362.05, LCMS method C.

### Example 38

### (1RS,3RS)—N—((S)-(3-chloro-2,6-difluorophenyl)(cyclopentyl)methyl)-3-(1H-imidazol-2-yl)cyclopentane-1-carboxamide and (1RS,3SR)—N—((S)-(3-chloro-2,6-difluorophenyl)(cyclopentyl)methyl)-3-(1H-imidazol-2-yl)cyclopentane-1-carboxamide

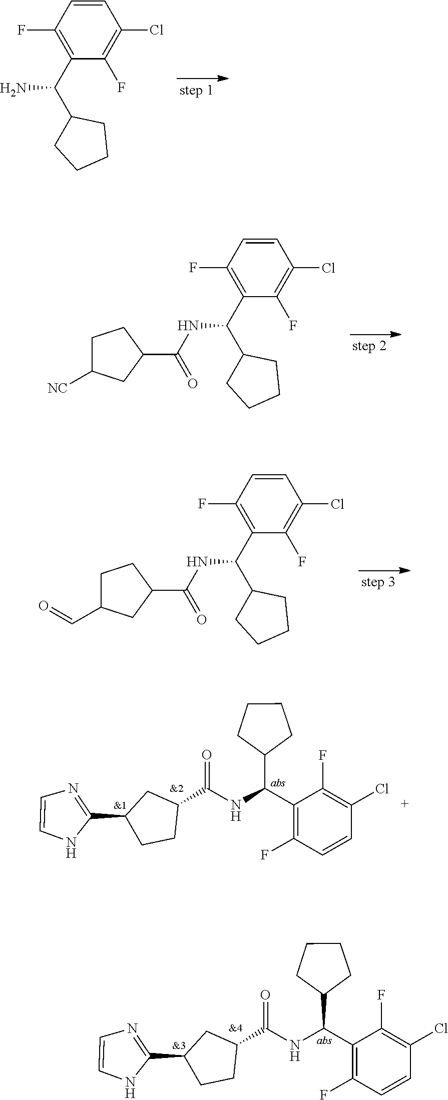 [SMILES: N[C@H](c1c(F)ccc(Cl)c1F)C1CCCC1 ; N#CC1CCC(C(=O)N[C@H](c2c(F)ccc(Cl)c2F)C2CCCC2)C1 ; O=CC1CCC(C(=O)N[C@H](c2c(F)ccc(Cl)c2F)C2CCCC2)C1 ; O=C(N[C@H](c1c(F)ccc(Cl)c1F)C1CCCC1)[C@@H]1CC[C@@H](c2ncc[nH]2)C1 ; O=C(N[C@H](c1c(F)ccc(Cl)c1F)C1CCCC1)[C@@H]1CC[C@@H](c2ncc[nH]2)C1]

### Step 1. Synthesis of N—((S)-(3-chloro-2,6-difluorophenyl)(cyclopentyl)methyl)-3-cyanocyclopentane-1-carboxamide

A flask equipped with a magnetic stirrer bar was charged with 3-cyanocyclopentane-1-carboxylic acid (160 mg, 1.15 mmol) and (S)-(3-chloro-2,6-difluorophenyl)(cyclopentyl)methanamine (324 mg, 1.15 mmol). DMF (2 mL) was added, followed by DIPEA (601 μL, 3.45 mmol) and T3P (1.10 g, 50% wt, 1.72 mmol) dropwise. The reaction mixture stirred at ambient temperature for 30 minutes. The reaction was diluted with EtOAc (10 mL) and H₂O (30 mL). The organic layer was washed twice with water, then saturated NH₄Cl solution, and finally brine. The organic layer was dried over Na₂SO₄ and concentrated under reduced pressure. The crude material was purified over a reverse phase column chromatography (mobile phase A: 10 mM ammonium formate in water, mobile phase B: acetonitrile; gradient: A:B 90:10 to 30:70) to give N—((S)-(3-chloro-2,6-difluorophenyl)(cyclopentyl)methyl)-3-cyanocyclopentane-1-carboxamide (320 mg). LCMS RT 1.79 min, [M+H]⁺ 367.2, RT 1.82 min, [M+H]⁺ 367.2, LCMS method L.

### Step 2. Synthesis of N—((S)-(3-chloro-2,6-difluorophenyl)(cyclopentyl)methyl)-3-formylcyclopentane-1-carboxamide

A flame-dried microwave vial equipped with a magnetic stirrer bar was charged with N—((S)-(3-chloro-2,6-difluorophenyl)(cyclopentyl)methyl)-3-cyanocyclopentane-1-carboxamide (100 mg, 273 μmol). DCM (2 mL) was added under a N₂ atmosphere and the reaction was cooled down to −78° C. Diisobutylaluminum hydride (654 μL, 1 M in DCM, 654 μmol) was added dropwise and the reaction was stirred for 40 mins at −78° C. The reaction was warmed up to room temperature, diluted with DCM and quenched with Rochelle salt solution (10 mL). The biphasic mixture was allowed to stir for 15 minutes and the aqueous layer was extracted with DCM twice. The organic layers were combined, dried over Na₂SO₄ and concentrated under reduced pressure to afford N—((S)-(3-chloro-2,6-difluorophenyl)(cyclopentyl)methyl)-3-formylcyclopentane-1-carboxamide as a colorless viscous oil (100 mg), which was used in the next step without purification. LCMS RT 1.81 min, [M+H]⁺ 370.2, LCMS method L.

### Step 3. Synthesis of (1RS,3RS)—N—((S)-(3-chloro-2,6-difluorophenyl)(cyclopentyl)methyl)-3-(1H-imidazol-2-yl)cyclopentane-1-carboxamide and (1RS,3SR)—N—((S)-(3-chloro-2,6-difluorophenyl)(cyclopentyl)methyl)-3-(1H-imidazol-2-yl)cyclopentane-1-carboxamide

To a solution of N—((S)-(3-chloro-2,6-difluorophenyl)(cyclopentyl)methyl)-3-formylcyclopentane-1-carboxamide (40.0 mg, 108 μmol) in ethanol (0.5 mL) at 0° C. was added a solution of glyoxal (18.6 μL, 40% wt. in water, 162 μmol) and NH₄OH (145 μL, 29% wt, 1.08 mmol). The reaction mixture was allowed to warm up to room temperature and stirred for 5 hours. The reaction mixture was diluted with EtOAc and water. The aqueous phase was extracted twice with EtOAc. The organic phases were combined, dried over Na₂SO₄ and concentrated in vacuo. The crude mixture was purified on a reverse phase column (30 g), eluent: 10 mM ammonium formate in water:acetonitrile 95:5 to 50:50 in 16 minutes to give the two racemates. Racemate 1: 5.3 mg; LCMS RT 2.79 min, [M+H]⁺ 408.3, LCMS method M.; ¹HNMR (400 MHz, DMSO-d6) δ 8.37 (d, J=7.4 Hz, 1H), 8.18 (s, 1H), 7.48 (app. td, J=8.5, 4.3 Hz, 1H), 7.08 (app. t, J=9.3 Hz, 1H), 6.80 (s, 1H), 6.79 (s, 1H), 4.78 (dd, J=10.8, 7.7 Hz, 1H), 3.18-3.05 (m, 1H), 2.89-2.79 (m, 1H), 2.42-2.34 (m, 1H), 2.03-1.62 (m, 6H), 1.62-1.36 (m, 5H), 1.35-1.16 (m, 2H), 1.01-0.87 (m, 1H). Racemate 2: 4.1 mg, 80:20 mixture of racemate 2:racemate 1. LCMS RT 2.97 min, [M+H]⁺ 408.3, LCMS method M. ¹HNMR (400 MHz, DMSO-d6) δ 8.87 (overlapping d, J=6.3 Hz, 1H), 8.86 (overlapping d, J=6.7 Hz, 1H), 8.28 (br. ss, 1H), 7.56-7.47 (m, 1H), 7.16-7.08 (m, 1H), 6.85 (overlapping br. s, 1H), 6.83 (overlapping br. s, 1H), 4.84 (br. dd, J=10.9, 7.6 Hz, 1H), 3.14 (overlapping m, 1H), 2.83-2.72 (m, 1H), 2.44 (overlapping m, 1H), 2.24-2.13 (m, 0.5H), 2.13-2.03 (m, 0.5H), 1.99-1.67 (m, 6H), 1.65-1.41 (m, 4H), 1.39-1.20 (m, 2H), 1.07-0.95 (m, 1H).

Additional compounds prepared according to the methods of Example 38 are listed in Table 6 below. Corresponding ¹H NMR and mass spectrometry characterization for these compounds are described in Table 1. Certain compounds in Table 6 below were prepared with other compounds whose preparation is described further below in the Examples.

TABLE 6
Additional exemplary compounds

| | |
|---|---|
| I-2055 | I-2058 |

### Example 39

### (1r,3S)—N—((S)-(2,3-dichloro-6-fluoro-5-hydroxyphenyl)(4-fluorobicyclo[2.2.1]heptan-1-yl)methyl)-3-((pyridazin-3-ylmethyl)amino)cyclobutane-1-carboxamide

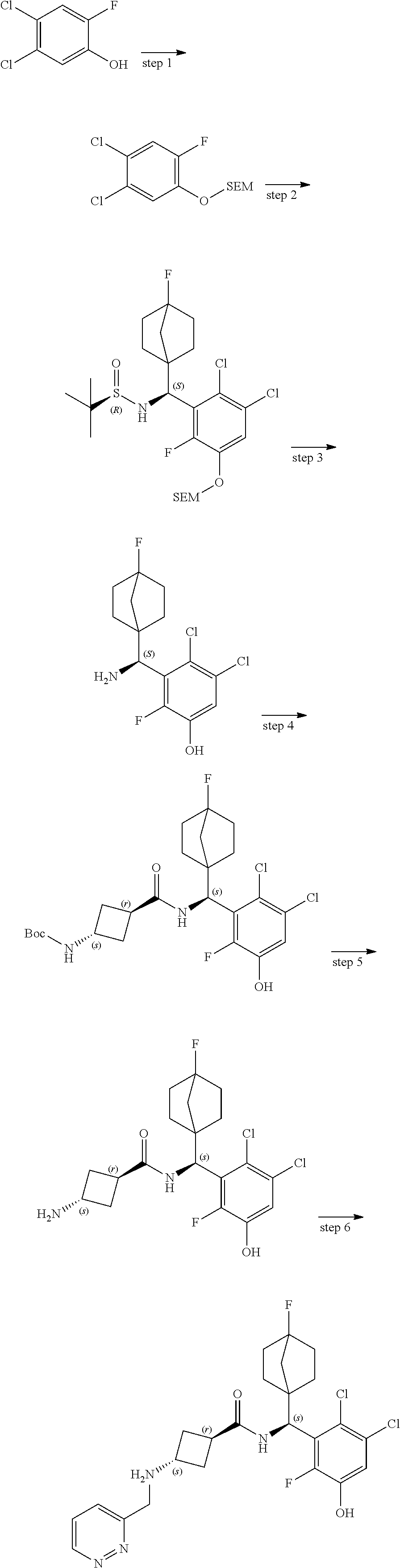

### Step 1. Synthesis of (2-((4,5-dichloro-2-fluorophenoxy)methoxy)ethyl)trimethylsilane

To a mixture of 4,5-dichloro-2-fluorophenol (7.5 g, 41.4 mmol) and K₂CO₃ (11.45 g, 82.9 mmol) in acetonitrile (75 mL) was added SEM-Cl (11.0 mL, 62.2 mmol) dropwise at 0° C. under a nitrogen atmosphere. The mixture was stirred for 1 hour at 25° C. The reaction was quenched with water (100 mL) and extracted with ethyl acetate (200 mL) three times. The combined organic layers were washed with brine, dried over sodium sulfate, filtered and concentrated in vacuo. The residue was purified by silica gel chromatography (30 g column; eluting with petroleum ether) to afford (2-((4,5-dichloro-2-fluorophenoxy)methoxy)ethyl)tri methylsilane (12 g, 39 mmol) as a colorless oil. ¹H NMR (400 MHz, DMSO-d₆) δ 7.77 (d, J=10.8 Hz, 1H), 7.58 (d, J=8.1 Hz, 1H), 5.39 (s, 2H), 3.81-3.72 (m, 2H), 0.96-0.83 (m, 2H), 0.03-0.01 (m, 9H).

### Step 2. Synthesis of (R)—N—((S)-(2,3-dichloro-6-fluoro-5-((2-(trimethylsilyl)ethoxy) methoxy)phenyl)(4-fluorobicyclo[2.2.1]heptan-1-yl)methyl)-2-methylpropane-2-sulfinamide

To a mixture of (2-((4,5-dichloro-2-fluorophenoxy)methoxy)ethyl)trimethylsilane (666 mg, 2.14 mmol) in THF (15 mL) was added LDA (1.53 mL, 2 M in THF, 3.06 mmol) dropwise at −60° C. under a nitrogen atmosphere. The mixture was stirred for 1 h at −60° C. prior to the addition of (R)—N-((4-fluorobicyclo[2.2.1]heptan-1-yl)methylene)-2-methylpropane-2-sulfinamide (500 mg, 2.04 mmol) at −60° C. The mixture was stirred for 1 h at room temperature. The reaction was quenched with saturated NH₄Cl (aq.). The reaction mixture was di luted with water (10 mL), and the aqueous phase was extracted with ethyl acetate (30 mL) three times. The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 10% to 50% B in 10 min; detector: UV 254 nm) to give (R)—N—((S)-(2,3-dichloro-6-fluoro-5-((2-(trimethylsilyl)ethoxy)methoxy)phenyl)(4-fluorobicyclo[2.2.1]heptan-1-yl)methyl)-2-methylpropane-2-sulfinamide (990 mg, 1.78 mmol). LCMS RT 1.440 min, [M+H]⁺ 556.15, LCMS method C.

### Step 3. Synthesis of (S)-3-(amino(4-fluorobicyclo[2.2.1]heptan-1-yl)methyl)-4,5-dichloro-2-fluorophenol

A mixture of (R)—N—((S)-(2,3-dichloro-6-fluoro-5-((2-(trimethylsilyl)ethoxy)methoxy)phenyl)(4-fluorobicyclo[2.2.1]heptan-1-yl)methyl)-2-methylpropane-2-sulfinamide (990 mg, 1.78 mmol) in HCl (10 mL, 4 N in 1,4-dioxane) was stirred for 1 h at room temperature. The mixture was concentrated to afford (S)-3-(amino(4-fluorobicyclo[2.2.1]heptan-1-yl)methyl)-4,5-dichloro-2-fluorophenol (543 mg, 1.69 mmol) as a yellow oil. LCMS RT 0.730 mi n, [M+H]⁺ 322.0, LCMS method C.

### Step 4. Synthesis of tert-butyl ((1S,3r)-3-(((S)-(2,3-dichloro-6-fluoro-5-hydroxyphenyl)(4-fluorobicyclo[2.2.1]heptan-1-yl)methyl)carbamoyl)cyclobutyl)carbamate

To a mixture of (S)-3-(amino(4-fluorobicyclo[2.2.1]heptan-1-yl)methyl)-4,5-dichloro-2-fluorophenol (150 mg, 466 μmol), (1r,3r)-3-((tert-butoxycarbonyl)amino)cyclobutane-1-carboxylic acid (100 mg, 466 μmol) and NaHCO₃ (117 mg, 1.40 mmol) in DMF (2 mL) was added HATU (266 mg, 698 μmol). The mixture was stirred for 1 h at room temperature. The reaction mixture was diluted with water (30 mL), and the aqueous phase was extracted with ethyl acetate (50 mL) three times. The combined organic layers were washed with brine, dried over sodium sulfate, filtered and concentrated in vacuo. The resulting crude material was purified by C18 flash to afford tert-butyl ((1S,3r)-3-(((S)-(2,3-dichloro-6-fluoro-5-hydroxyphenyl)(4-fluorobicyclo[2.2.1]heptan-1-yl)methyl)carbamoyl)cyclobutyl)carbamate (178 mg, 343 μmol) as a colorless oil. LCMS RT 1.127 min, [M+H]⁺ 463, LCMS method C.

### Step 5. Synthesis of (1r,3S)-3-amino-N—((S)-(2,3-dichloro-6-fluoro-5-hydroxyphenyl)(4-fluorobicyclo[2.2.1]heptan-1-yl)methyl)cyclobutane-1-carboxamide

A mixture of ((1S,3r)-3-(((S)-(2,3-dichloro-6-fluoro-5-hydroxyphenyl)(4-fluorobicyclo[2.2.1]heptan-1-yl)methyl)carbamoyl)cyclobutyl)carbamate (158 mg, 304 μmol) in HCl (3 mL, 4 N in dioxane) was stirred for 1 h at 25° C. The mixture was concentrated and the residue was diluted with saturated NaHCO₃ solution. The reaction mixture was extracted with ethyl acetate (50 mL) three times. The combined organic layers were washed with brine, dried over sodium sulfate, filtered and concentrated in vacuo to afford (1r,3S)-3-amino-N—((S)-(2,3-dichloro-6-fluoro-5-hydroxyphenyl)(4-fluorobicyclo[2.2.1]heptan-1-yl)methyl)cyclobutane-1-carboxamide (90 mg, 0.21 mmol) as an off-white solid. LCMS RT 0.431 min, [M+H]⁺ 419. LCMS method C.

### Step 6. Synthesis of (1r,3S)—N—((S)-(2,3-dichloro-6-fluoro-5-hydroxyphenyl)(4-fluorobicyclo[2.2.1]heptan-1-yl)methyl)-3-((pyridazin-3-ylmethyl)amino)cyclobutane-1-carboxamide

A mixture of pyridazine-3-carbaldehyde (7.7 mg, 72 μmol) and (1r,3S)-3-amino-N—((S)-(2,3-dichloro-6-fluoro-5-hydroxyphenyl)(4-fluorobicyclo[2.2.1]heptan-1-yl)methyl)cyclobutane-1-carboxamide (30 mg, 72 μmol) in MeOH (1 mL) was stirred for 0.5 h at 2° C. prior to the addition of NaBH₃CN (5.4 mg, 85 μmol). The mixture was stirred for 1 h at 25° C. The mixture was diluted with water (20 mL) and extracted with ethyl acetate (50 mL) three times. The combined organic layers were washed with brine, dried over sodium sulfate, filtered and concentrated in vacuo. The resulting crude material was purified by preparative HPLC (column: XBridge Shield RP18 OBD Column, 30*150 mm, 5 μm; mobile phase A: water (10 mM NH₄HCO₃+0.10% NH₄OH), mobile phase B: MeOH; flow rate: 60 mL/min; gradient: 38% B to 56% B in 11 min; wavelength: 220/254 nm; RT (min): 10.6; injection volume: 0.475 mL) to give (1r,3S)—N—((S)-(2,3-dichloro-6-fluoro-5-hydroxyphenyl)(4-fluorobicyclo[2.2.1]heptan-1-yl)methyl)-3-((pyridazin-3-ylmethyl)amino)cyclobutane-1-carboxamide (16 m g, 31 μmol) as an off-white amorphous solid. ¹H NMR (400 MHz, DMSO-d₆) δ 9.11 (dd, J=4.7, 1.9 Hz, 1H), 8.04 (d, J=8.4 Hz, 1H), 7.70 (dd, J=8.5, 1.9 Hz, 1H), 7.65 (dd, J=8.5, 4.7 Hz, 1H), 7.10 (d, J=8.1 Hz, 1H), 6.53 (s, 1H), 6.29 (s, 1H), 5.51-5.33 (m, 1H), 3.93 (s, 2H), 3.32 (t, J=7.4 Hz, 1H), 3.11-3.02 (m, 1H), 2.18 (dq, J=7.8, 4.1, 3.2 Hz, 1H), 2.10-1.88 (m, 3H), 1.81-1.52 (dd, J=22.7, 10.1 Hz, 1OH). LCMS RT 0.872 min, [M+H]⁺ 511.15, LCMS method B.

Additional compounds prepared according to the methods of Example 39 are listed in Table 7 below. Corresponding ¹H NMR and mass spectrometry characterization for these compounds are described in Table 1. Certain compounds in Table 7 below were prepared with other compounds whose preparation is described further below in the Examples.

TABLE 7
Additional exemplary compounds

| | | | |
|---|---|---|---|
| I-2992 | I-2993 | I-2994 | I-2995 |

### Example 40

### (1R,3R,4S)—N—((S)-(3-chloro-2,6-difluorophenyl)(4-fluorobicyclo[2.2.1]heptan-1-yl)methyl)-3-(ethylsulfonamido)-4-hydroxycyclopentane-1-carboxamide and (1S,3R,4S)—N—((S)-(3-chloro-2,6-difluorophenyl)(4-fluorobicyclo[2.2.1]heptan-1-yl)methyl)-3-(ethylsulfonamido)-4-hydroxycyclopentane-1-carboxamide

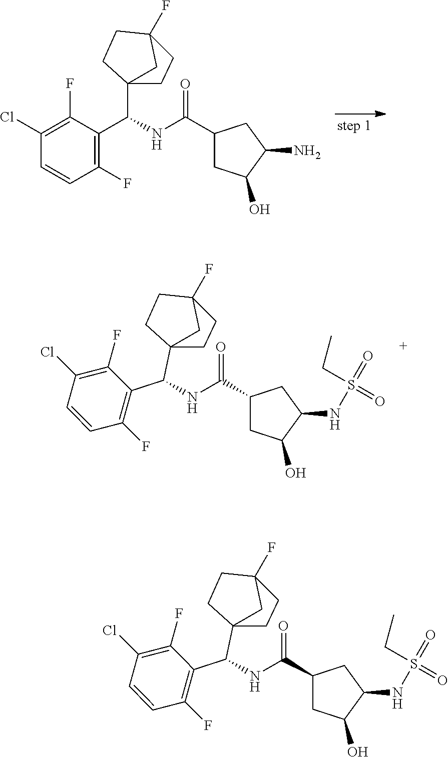 [SMILES: N[C@@H]1CC(C(=O)N[C@H](c2c(F)ccc(Cl)c2F)C23CCC(F)(CC2)C3)C[C@@H]1O ; CCS(=O)(=O)N[C@@H]1C[C@@H](C(=O)N[C@H](c2c(F)ccc(Cl)c2F)C23CCC(F)(CC2)C3)C[C@@H]1O ; CCS(=O)(=O)N[C@@H]1C[C@H](C(=O)N[C@H](c2c(F)ccc(Cl)c2F)C23CCC(F)(CC2)C3)C[C@@H]1O]

### Step 1. Synthesis of (1R,3R,4S)—N—((S)-(3-chloro-2,6-difluorophenyl)(4-fluorobicyclo[2.2.1]heptan-1-yl)methyl)-3-(ethylsulfonamido)-4-hydroxycyclopentane-1-carboxamide and (1S,3R,4S)—N—((S)-(3-chloro-2,6-difluorophenyl)(4-fluorobicyclo[2.2.1]heptan-1-yl)methyl)-3-(ethylsulfonamido)-4-hydroxycyclopentane-1-carboxamide

To a mixture of (3R,4S)-3-amino-N—((S)-(3-chloro-2,6-difluorophenyl)(4-fluorobicyclo[2.2.1]heptan-1-yl)methyl)-4-hydroxycyclopentane-1-carboxamide (50 mg, 0.12 mmol) and DIEA (63 μL, 0.36 mmol) in DCM (2 mL) was added ethanesulfonyl chloride (19 mg, 0.14 mmol) dropwise at 0° C. The mixture was stirred for 1 h at 25° C. The mixture was concentrated in vacuum. The resulting crude material was purified by preparative HPLC (column: XBridge Prep OBD C18 Column, 30*150 mm, 5 μm; mobile phase A: water (10 mM NH₄HCO₃+0.05% NH40H), mobile phase B: acetonitrile; flow rate: 60 mL/min; gradient: 38% B to 52% B in 7 min; wavelength: 254/220 nm; RT (min): 7.45) to afford (3R,4S)—N—((S)-(3-chloro-2,6-difluorophenyl)(4-fluorobicyclo[2.2.1]heptan-1-yl)methyl)-3-(ethylsulfonamido)-4-hydroxycyclopentane-1-carboxamide (40 mg, 65 μmol) as a white amorphous solid. LCMS RT 1.098 min, [M+H]⁺ 509.05, LCMS method B.

The product was further purified by preparative chiral HPLC (column: CHIRALPAK ID, 2*25 cm, 5 μm; mobile phase A: hexane (0.5% 2M NH₃ in MeOH), mobile phase B: EtOH:DCM 1:1; flow rate: 20 mL/min; gradient: 30% isocratic; wavelength: 220/254 nm; RT1 (min): 6.94; RT2 (min): 11.38; sample solvent: EtOH:DCM 1:1; injection volume: 1.9 mL) to afford (1R,3R,4S)—N—((S)-(3-chloro-2,6-difluorophenyl)(4-fluorobicyclo[2.2.1]heptan-1-yl)methyl)-3-(ethylsulfonamido)-4-hydroxycyclopentane-1-carboxamide and (1S,3R,4S)—N—((S)-(3-chloro-2,6-difluorophenyl)(4-fluorobicyclo[2.2.1]heptan-1-yl)methyl)-3-(ethylsulfonamido)-4-hydroxycyclopentane-1-carboxamide, both as a white amorphous solid.

Isomer 1: 4 mg, 8 μmol. LCMS RT 1.094 min, [M+H]⁺ 509.10, LCMS method B.

Isomer 2: 5.2 mg, 10 μmol. LCMS RT 1.092 min, [M+H]⁺ 509.10, LCMS method B.

### Example 41

### (S)—N-((3-chloro-2,6-difluorophenyl)(cyclopentyl)methyl)-1-phenylmethanesulfonamide

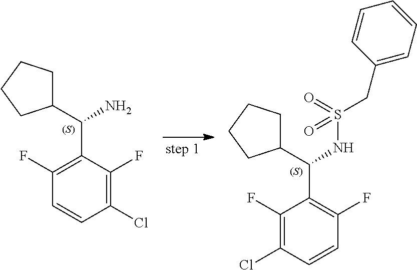

### Step 1. Synthesis of (S)—N-((3-chloro-2,6-difluorophenyl) (cyclopentyl)methyl)-1-phenylmethanesulfonamide

To a mixture of (S)-(3-chloro-2,6-difluorophenyl) (cyclopentyl)methanamine (100 mg, 407 μmol) and TEA (206 mg, 2.04 mmol) in DCM (1 mL) was added phenylmethanesulfonyl chloride (93.1 mg, 488 μmol) at room temperature. The mixture was stirred for 1 hour at room temperature. The reaction was quenched with MeOH (1 ml) and concentrated. The residue was first purified by reverse phase flash chromatography (column: C18 silica gel; mobile phase A: water, mobile phase B: acetonitrile; gradient: 10% to 60% B in 10 min; detector: UV 220 nm), then purified by preparative HPLC (column: XBridge Prep OBD C18 Column, 30*150 mm, 5 μm; mobile phase A: water (10 mM NH₄HCO₃), mobile phase B: acetonitrile; flow rate: 60 mL/min; gradient: 50% B to 80% B in 7 min, then 80% B; wavelength: 220 nm; RT1 (min): 6.6) to give (S)—N-((3-chloro-2,6-difluorophenyl) (cyclopentyl)methyl)-1-phenylmethanesulfonamide (32.1 mg, 80 μmol) as an off-white amorphous solid. ¹H NMR (400 MHz, DMSO-d6) δ 7.91 (d, J=7.8 Hz, 1H), 7.57 (td, J=8.7, 5.5 Hz, 1H), 7.30-7.21 (m, 3H), 7.19-7.10 (m, 3H), 4.42 (dd, J=10.7, 7.7 Hz, 1H), 4.26-4.07 (i, 2H), 2.39 (p, J=8.6 Hz, 1H), 1.92 (dp, J=12.3, 6.8, 5.8 Hz, 1H), 1.69-1.34 (m, 5H), 1.32-1.19 (i, 1H), 0.92 (dq, J=12.2, 8.0 Hz, 1H). LCMS RT 1.692 min [M+Na]⁺ 422, LCMS method C.

Additional compounds prepared according to the methods of Examples 40-41 are listed in Table 8 below. Corresponding ¹H NMR and mass spectrometry characterization for these compounds are described in Table 1. Certain compounds in Table 8 below were prepared with other compounds whose preparation is described further below in the Examples.

TABLE 8
Additional exemplary compounds

| | | | | |
|---|---|---|---|---|
| I-1816 | I-1827 | I-1959 | I-2009 | I-2151 |
| I-2152 | I-2153 | I-2681 | I-2683 | I-2694 |
| I-2695 | I-2701 | I-2702 | I-2703 | I-2704 |
| I-2705 | I-2706 | I-2707 | I-2748 | I-2749 |
| I-2750 | I-2751 | I-2766 | I-2767 | I-2768 |
| I-2769 | I-2819 | I-2820 | I-2821 | I-2822 |
| I-2847 | I-2848 | I-2849 | I-2850 | I-2851 |
| I-2852 | I-2853 | I-2854 | I-2855 | I-2856 |
| I-2882 | I-2883 | I-2896 | I-2897 | I-2898 |
| I-2899 | I-2900 | I-2901 | I-2907 | I-2908 |
| I-2909 | I-2910 | I-2911 | I-2912 | I-2913 |
| I-2914 | I-2930 | I-2931 | I-2932 | I-2933 |
| I-2934 | I-2935 | I-2936 | I-2937 | I-2938 |
| I-2939 | I-2940 | I-2941 | I-2968 | I-2969 |
| I-2970 | I-2971 | I-2972 | I-2986 | I-2987 |
| I-2988 | I-3030 | I-3031 | I-3032 | I-3033 |
| I-3034 | I-3035 | I-3036 | I-3037 | I-3038 |
| I-3039 | I-3040 | I-3041 | I-3042 | I-3043 |
| I-3066 | I-3072 | I-3074 | I-3075 | I-3076 |
| I-3077 | I-3078 | I-3079 | I-3080 | I-3090 |
| I-3097 | I-3107 | I-3108 | I-3114 | I-3116 |
| I-3117 | I-3133 | I-3135 | I-3136 | I-3142 |
| I-3143 | I-3155 | I-3158 | I-3159 | I-3162 |
| I-3163 | I-3165 | I-3199 | I-3200 | I-3201 |
| I-3205 | I-3206 | I-3207 | I-3208 | I-3209 |
| I-3210 | I-3211 | I-3229 | I-3230 | I-3231 |
| I-3236 | I-3237 | I-3295 | I-3296 | I-3299 |
| I-3300 | I-3301 | I-3302 | I-3303 | I-3304 |
| I-3305 | I-3306 | I-3307 | I-3308 | I-3309 |
| I-3310 | I-3311 | I-3312 | I-3313 | I-3314 |
| I-3315 | I-3322 | I-3323 | I-3330 | I-3343 |
| I-3344 | I-3345 | I-3346 | I-3347 | I-3348 |
| I-3349 | I-3360 | I-3361 | I-3362 | I-3363 |
| I-3364 | I-3365 | I-3366 | I-3368 | I-3369 |
| I-3370 | I-3371 | I-3372 | I-3399 | I-3400 |
| I-3407 | I-3412 | I-3413 | I-3418 | I-3419 |
| I-3420 | I-3421 | I-3422 | I-3424 | I-3445 |
| I-3446 | I-3447 | I-3448 | I-3452 | I-3453 |
| I-3454 | I-3455 | I-3456 | I-3457 | I-3458 |
| I-3459 | I-3460 | I-3461 | I-3462 | I-3463 |
| I-3464 | I-3465 | I-3466 | I-3467 | I-3468 |
| I-3469 | I-3470 | I-3471 | I-3472 | I-3473 |
| I-3474 | I-3475 | I-3476 | I-3477 | I-3478 |
| I-3480 | I-3481 | I-3482 | I-3483 | I-3484 |
| I-3485 | I-3486 | I-3517 | I-3520 | I-3523 |
| I-3524 | I-3525 | I-3549 | I-3550 | I-3551 |
| I-3552 | I-3553 | I-3554 | I-3555 | I-3556 |
| I-3557 | I-3558 | I-3559 | I-3560 | I-3561 |
| I-3562 | I-3563 | I-3564 | I-3565 | I-3566 |
| I-3567 | I-3568 | I-3569 | I-3570 | I-3571 |
| I-3572 | I-3573 | I-3574 | I-3575 | I-3576 |
| I-3577 | I-3578 | I-3579 | I-3580 | I-3581 |
| I-3582 | I-3583 | I-3584 | I-3585 | I-3594 |
| I-3601 | I-3602 | I-3603 | I-3604 | I-3611 |
| I-3612 | I-3613 | I-3614 | I-3631 | I-3632 |
| I-3633 | I-3651 | I-3652 | I-3653 | I-3654 |
| I-3655 | I-3656 | I-3657 | I-3658 | I-3659 |
| I-3660 | I-3661 | I-3662 | I-3663 | I-3664 |
| I-3665 | I-3666 | I-3667 | I-3668 | I-3669 |
| I-3670 | I-3672 | I-3684 | I-3685 | I-3686 |
| I-3687 | I-3714 | I-3715 | I-3716 | I-3741 |
| I-3742 | I-3743 | I-3744 | I-3745 | |

### Example 42

### N-(2-(3,4-dichlorophenyl)-2-methylpropyl)quinolin-2-amine

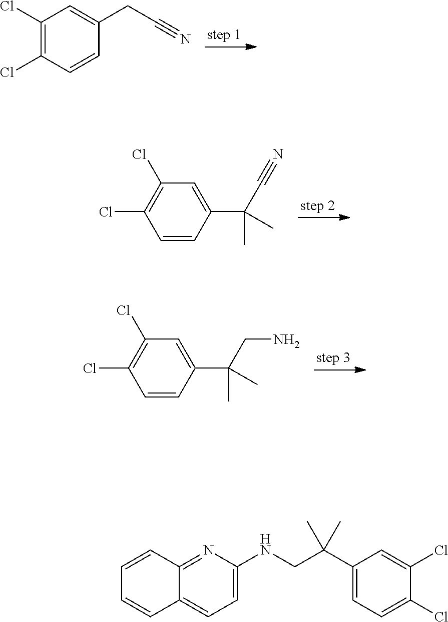 [SMILES: N#CCc1ccc(Cl)c(Cl)c1 ; CC(C)(C#N)c1ccc(Cl)c(Cl)c1 ; CC(C)(CN)c1ccc(Cl)c(Cl)c1 ; CC(C)(CNc1ccc2ccccc2n1)c1ccc(Cl)c(Cl)c1]

### Step 1. Synthesis of 2-(3,4-dichlorophenyl)-2-methylpropanenitrile

To a mixture of 2-(3,4-dichlorophenyl)acetonitrile (1000 mg, 5.38 mmol) in THF (15 mL) was added LiHMDS (13.4 mL, 1 M in THF, 13.4 mmol) dropwise at 0° C. under a nitrogen atmosphere. The mixture was stirred for 1 h at 0° C. prior to the addition of Mel (1.91 g, 13.4 mmol). The mixture was stirred for 1 hour at 25° C. The reaction was quenched with saturated NH₄Cl (aq.). The mixture was diluted with water (20 mL), and the aqueous phase was extracted with ethyl acetate (50 mL) three times. The combined organic layers were washed with brine, dried over sodium sulfate, filtered and concentrated in vacuo. The residue was purified by preparative TLC (petroleum ether/ethyl acetate, ratio 5/1) to afford 2-(3,4-dichlorophenyl)-2-methylpropanenitrile (1.0 g, 4.67 mmol) as a yellow oil. ¹HNMR (400 MHz, DMSO-d6) 7.78 (d, J=2.4 Hz, 1H), 7.71 (d, J=8.5 Hz, 1H), 7.54 (dd, J=8.5, 2.3 Hz, 1H), 1.70 (s, 6H).

### Step 2. Synthesis of 2-(3,4-dichlorophenyl)-2-methylpropan-1-amine

To a mixture of 2-(3,4-dichlorophenyl)-2-methylpropanenitrile (1.0 g, 4.67 mmol) in THF (10 mL) was added LiAlH₄ (213 mg, 5.60 mmol) in portions at 0° C. under a nitrogen atmosphere. The mixture was stirred for 2 hours at 80° C. The reaction was then cooled to 0° C. and quenched with water (3 mL), sodium hydroxide (6 mL, 4 N in water) and water (3 mL). The reaction mixture was filtered through a pad of Celite, the pad was washed with ethyl acetate, and the filtrate was concentrated in vacuo to give 2-(3,4-dichlorophenyl)-2-methylpropan-1-amine (900 mg, 1.38 mmol) as a yellow oil. LCMS RT 0.526 min, [M+H]⁺ 218, LCMS method C.

### Step 3. Synthesis of N-(2-(3,4-dichlorophenyl)-2-methylpropyl)quinolin-2-amine

A mixture of 2-chloroquinoline (200 mg, 1.22 mmol), 2-(3,4-dichlorophenyl)-2-methylpropan-1-amine (266 mg, 1.22 mmol), Pd₂(dba)₃ (111 mg, 122 μmol), BINAP (151 mg, 244 μmol) and t-BuONa (117 mg, 1.22 mmol) in toluene (4 mL) was stirred at 80° C. for 1 h. The mixture was diluted with water (20 ml) and extracted with ethyl acetate (20 ml*3). The combined organic layers were washed with brine, dried over Na₂SO₄ and concentrated. The residue was purified first by preparative TLC (MeOH:DCM 1:10) and then by preparative HPLC (column: XBridge Shield RP18 OBD Column, 30*150 mm, 5 μm; mobile phase A: water (10 mM NH₄HCO₃), mobile phase B: acetonitrile; flow rate: 60 mL/min; gradient: 55% B to 85% B in 7 min, then 85% B; wavelength: 254 nm; RT (min): 7.48) to give N-[2-(3,4-dichlorophenyl)-2-methylpropyl]quinolin-2-amine (91.3 mg, 264 μmol) as a colorless oil. ¹H NMR (400 MHz, DMSO-d6) δ 7.78 (d, J=8.9 Hz, 1H), 7.68 (d, J=2.2 Hz, 1H), 7.61-7.51 (m, 2H), 7.51-7.40 (m, 3H), 7.12 (ddd, J=8.0, 6.6, 1.6 Hz, 1H), 6.84-6.72 (m, 2H), 3.71 (d, J=5.8 Hz, 2H), 1.36 (s, 6H). LCMS RT 0.855 min, [M+H]⁺ 345.00, LCMS method C.

### Example 43

### 2-(3-(((1-(3-chlorophenyl)cyclobutyl)methyl)amino)-1H-pyrazol-1-yl)-N-methylacetamide

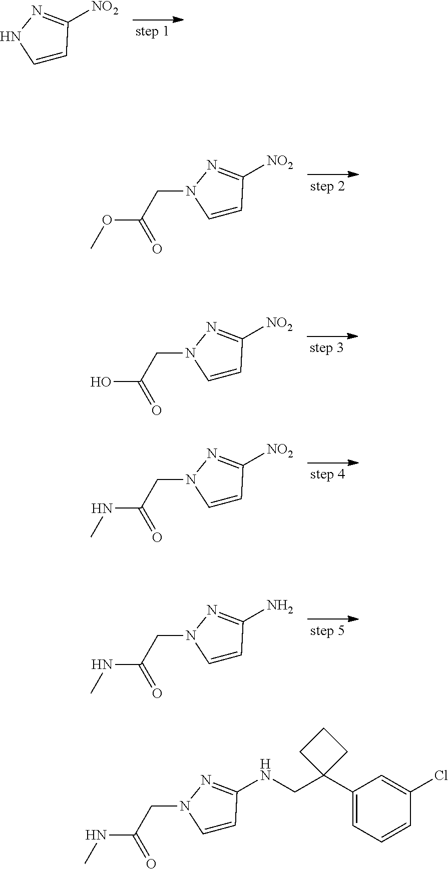 [SMILES: O=[N+]([O-])c1cc[nH]n1 ; COC(=O)Cn1ccc([N+](=O)[O-])n1 ; O=C(O)Cn1ccc([N+](=O)[O-])n1 ; CNC(=O)Cn1ccc([N+](=O)[O-])n1 ; CNC(=O)Cn1ccc(N)n1 ; CNC(=O)Cn1ccc(NCC2(c3cccc(Cl)c3)CCC2)n1]

### Step 1. Synthesis of methyl 2-(3-nitro-1H-pyrazol-1-yl)acetate

To a solution of 3-nitro-1H-pyrazole (5.00 g, 44 mmol) in DMF (30.0 mL) was added methyl 2-bromoacetate (4.18 mL, 44.2 mmol) and K₂CO₃ (12.2 g, 88.4 mmol). Then the mixture was stirred at 25° C. for 16 hours. The mixture was poured into water (30.0 mL) and extracted with ethyl acetate (30.0 mL*5). The combined organic layers were washed with brine (30.0 mL), dried over Na₂SO₄ and concentrated under vacuum. The residue was purified by column chromatography (SiO₂, petroleum ether/ethyl acetate 1/1) to give methyl 2-(3-nitro-1H-pyrazol-1-yl)acetate (6.28 g, 28.5 mmol) as a yellow oil. ¹HNMR (400 MHz, DMSO-d₆) δ 8.05 (d, J=2.6 Hz, 1H), 8.03-7.99 (m, 1H), 7.11-7.05 (m, 1H), 7.04-6.99 (m, 1H), 5.29 (s, 2H), 3.72 (s, 3H).

### Step 2. Synthesis of 2-(3-nitro-1H-pyrazol-1-yl)acetic acid

To a solution of methyl 2-(3-nitro-1H-pyrazol-1-yl)acetate (4.00 g, 18.1 mmol) in THF (40.0 mL) and H₂O (8.00 mL) was added LiOH H₂O (3.81 g, 90.8 mmol). The mixture was stirred at 60° C. for 16 hours. Ethyl acetate (10.0 mL) and water (10.0 mL) were added and the layers were separated. The pH of the aqueous phase was adjusted to 2 with 1N HCl, and the mixture was extracted with ethyl acetate (10.0 mL*3). Combined extracts were washed with brine (10.0 mL) and dried over Na₂SO₄. The mixture was filtered and concentrated under vacuum to give 2-(3-nitro-1H-pyrazol-1-yl)acetic acid (2.45 g, 14.3 mmol) as a yellow solid. ¹H NMR: (400 MHz, DMSO-d₆) δ 14.04-13.85 (m, 1H), 8.03 (s, 1H), 7.07 (d, J=2.4 Hz, 1H), 5.15 (s, 2H).

### Step 3. Synthesis of N-methyl-2-(3-nitro-1H-pyrazol-1-yl)acetamide

To a solution of 2-(3-nitro-1H-pyrazol-1-yl)acetic acid (2.00 g, 11.7 mmol) in DMF (20.0 mL) was added methanamine HCl salt (1.58 g, 23.3 mmol), HATU (5.78 g, 15.1 mmol) and DIEA (10.1 mL, 58.4 mmol). The mixture was stirred at 25° C. for 16 hours. The combined mixture was poured into water (20.0 mL) and extracted with ethyl acetate (20.0 mL*3). The combined organic layers were washed with brine (20.0 mL*3), dried over Na₂SO₄ and concentrated under vacuum. The residue was purified by silica gel column chromatography (petroleum ether/ethyl acetate 1/1) to give N-methyl-2-(3-nitro-1H-pyrazol-1-yl)acetamide (900 mg, 4.89 mmol) as a yellow oil. ¹H NMR: (400 MHz, DMSO-d₆) δ 8.21-8.13 (m, 1H), 7.99 (d, J=2.4 Hz, 1H), 7.05 (d, J=2.4 Hz, 1H), 4.95-4.93 (m, 2H), 2.64 (d, J=4.4 Hz, 3H).

### Step 4. Synthesis of 2-(3-amino-1H-pyrazol-1-yl)-N-methylacetamide

To a solution of N-methyl-2-(3-nitro-1H-pyrazol-1-yl)acetamide (800 mg, 4.34 mmol) in MeOH (10.0 mL) was added Pd/C (0.10 g, 10%) under a N₂ atmosphere. The suspension was degassed and purged with H₂ three times. The mixture was stirred under H₂ (15 psi) at 25° C. for 3 hours. The mixture was filtered and the filtrate was concentrated to give 2-(3-amino-1H-pyrazol-1-yl)-N-methylacetamide (544 mg, 3.53 mmol) as a colorless oil. ¹H NMR: (400 MHz, DMSO-d₆) δ 7.71 (br s, 1H), 7.30 (d, J=2.4 Hz, 1H), 5.40 (d, J=2.0 Hz, 1H), 4.42 (s, 2H), 2.59 (d, J=4.4 Hz, 3H).

### Step 5. Synthesis of 2-(3-(((1-(3-chlorophenyl)cyclobutyl)methyl)amino)-1H-pyrazol-1-yl)-N-methylacetamide

To a solution of 2-(3-amino-1H-pyrazol-1-yl)-N-methylacetamide (540 mg, 3.50 mmol) in MeOH (5.0 mL) was added 1-(3-chlorophenyl)cyclobutane-1-carbaldehyde (681 mg, 3.50 mmol). The mixture was stirred at 20° C. for 1 hour. NaBH₃CN (1.10 g, 17 mmol) was added at 0° C., and the mixture was stirred at 20° C. for 15 hours. The mixture was concentrated and the residue was purified by preparative HPLC (column: waters Xbridge 150*25 mm, 5 μm; mobile phase A: water (0.05% ammonia hydroxide v/v), mobile phase B: acetonitrile; gradient: 30%-60% B over 9 min) to give 2-(3-(((1-(3-chlorophenyl)cyclobutyl)methyl)amino)-1H-pyrazol-1-yl)-N-methylacetamide (281 mg, 838 umol) as a white solid. ¹H NMR: (400 MHz, DMSO-d₆)

δ 7.66 (br d, J=4.4 Hz, 1H), 7.36-7.28 (m, 2H), 7.24-7.17 (m, 2H), 7.17-7.12 (m, 1H), 5.37 (d, J=2.4 Hz, 1H), 4.86 (t, J=6.4 Hz, 1H), 4.42 (s, 2H), 3.31 (d, J=6.4 Hz, 2H), 2.58 (d, J=4.8 Hz, 3H), 2.31-2.14 (m, 4H), 2.10-1.97 (m, 1H), 1.84-1.71 (m, 1H).

### Example 44

### 6-(((1-(3,4-dichlorophenyl)cyclobutyl)methyl)amino)-N-methylpyridazine-3-carboxamide

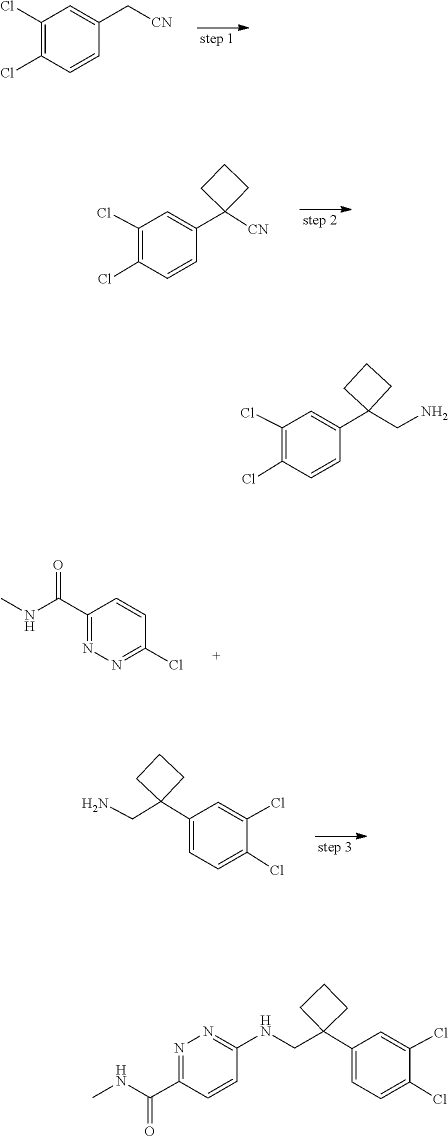 [SMILES: N#CCc1ccc(Cl)c(Cl)c1 ; N#CC1(c2ccc(Cl)c(Cl)c2)CCC1 ; NCC1(c2ccc(Cl)c(Cl)c2)CCC1 ; CNC(=O)c1ccc(Cl)nn1 ; NCC1(c2ccc(Cl)c(Cl)c2)CCC1 ; CNC(=O)c1ccc(NCC2(c3ccc(Cl)c(Cl)c3)CCC2)nn1]

### Step 1. Synthesis of 1-(3,4-dichlorophenyl)cyclobutane-1-carbonitrile

To a suspension of NaH (60% in mineral oil, 26.9 g, 672 mmol) in THF (100 mL) was added a solution of 2-(3,4-dichlorophenyl)acetonitrile (50.0 g, 269 mmol) in THF (200 mL) dropwise at 0° C. The mixture was stirred at 0° C. for 1 hour. Then 1,3-dibromopropane (57.0 g, 282 mmol) was added dropwise over 1.5 hours at 0° C. The mixture was warmed to 25° C. and stirred at 25° C. for 0.5 hr. The mixture was poured into saturated NH₄Cl solution (400 mL) and filtered. The filtrate was extracted with ethyl acetate (250 mL*3). The organic layers were washed with brine (250 mL), dried over Na₂SO₄, filtered and concentrated. The crude product was purified by silica gel column chromatography (petroleum ether:ethyl acetate 50:1 to 6:1) to give 1-(3,4-dichlorophenyl)cyclobutane-1-carbonitrile (22.1 g, 96.0 mmol) as a colorless oil. ¹H NMR: (400 MHz, CDCl₃) δ 7.55 (d, J=2.0 Hz, 1H), 7.52 (d, J=8.0 Hz, 1H), 7.32-7.29 (m, 1H), 2.90-2.85 (m, 2H), 2.64-2.62 (m, 2H), 2.61-2.47 (m, 1H), 2.15-2.09 (m, 1H).

### Step 2. Synthesis of (1-(3,4-dichlorophenyl)cyclobutyl)methanamine

To a suspension of LiAlH₄ (4.36 g, 115 mmol) in THF (100 mL) was added a solution of 1-(3,4-dichlorophenyl)cyclobutane-1-carbonitrile (20.0 g, 88.5 mmol) in THF (50.0 mL) dropwise at 0° C. The mixture was warmed to 25° C. and stirred at 25° C. for 1 hour. The stirring mixture was cooled to 10° C. Water (5.00 mL) was added, followed by 15% NaOH solution (5.00 mL), water (15.0 mL), and Na₂SO₄ (6.0 g). The mixture was filtered through celite. The filtrate was extracted with ethyl acetate (50.0 mL*2). The combined organic layers were washed with brine, dried over Na₂SO₄, filtered and concentrated to give (1-(3,4-dichlorophenyl)cyclobutyl)methanamine (11.0 g, 46.3 mmol) as a yellow oil. ¹H NMR: (400 MHz, CDCl₃) δ 7.28 (d, J=8.4 Hz, 1H), 7.19 (d, J=2.0 Hz, 1H), 6.96-6.94 (m, 1H), 2.93 (s, 2H), 2.31-2.26 (m, 2H), 2.16-2.11 (m, 2H), 2.09-2.02 (m, 1H), 1.92-1.84 (m, 1H).

### Step 3. Synthesis of 6-(((1-(3,4-dichlorophenyl)cyclobutyl)methyl)amino)-N-methylpyridazine-3-carboxamide

In a vial 6-chloro-N-methylpyridazine-3-carboxamide (25 mg, 0.15 mmol) and (1-(3,4-dichlorophenyl)cyclobutyl)methanamine (34 mg, 0.15 mmol) were dissolved in NMP (0.5 mL). DIEA (38 μL, 0.22 mmol) was added. The vial was sealed and heated at 100° C. over the weekend. After cooling to room temperature, the reaction was purified on AccQprep using 35-65% of acetonitrile (0.1% formic acid) in water to give 6-(((1-(3,4-dichlorophenyl)cyclobutyl)methyl)amino)-N-methylpyridazine-3-carboxamide (22 mg, 60 μmol). LCMS: RT 1.426 min, [M+H]⁺ 365.25. LCMS method K.

Additional compounds prepared according to the methods of Examples 42-44 are listed in Table 9 below. Corresponding ¹H NMR and mass spectrometry characterization for these compounds are described in Table 1. Certain compounds in Table 9 below were prepared with other compounds whose preparation is described further below in the Examples.

TABLE 9
Additional Exemplary Compounds that can be synthesized
similarly using Buchwald, reductive amination,
urea formation, or amide coupling reactions

| | | | | |
|---|---|---|---|---|
| I-3 | I-6 | I-180 | I-181 | I-191 |
| I-192 | I-193 | I-194 | I-198 | I-199 |
| I-200 | I-201 | I-202 | I-203 | I-209 |
| I-210 | I-211 | I-212 | I-213 | I-214 |
| I-216 | I-217 | I-218 | I-222 | I-358 |
| I-360 | I-362 | I-364 | I-366 | I-367 |
| I-376 | I-377 | I-411 | I-429 | I-430 |
| I-437 | I-438 | I-439 | I-440 | I-441 |
| I-442 | I-470 | I-471 | I-475 | I-476 |
| I-477 | I-478 | I-488 | I-489 | I-490 |
| I-492 | I-504 | I-505 | I-506 | I-508 |
| I-509 | I-511 | I-512 | I-513 | I-514 |
| I-524 | I-526 | I-527 | I-528 | I-536 |
| I-537 | I-538 | I-539 | I-540 | I-541 |
| I-542 | I-543 | I-544 | I-545 | I-546 |
| I-547 | I-549 | I-550 | I-551 | I-559 |
| I-560 | I-561 | I-562 | I-569 | I-570 |
| I-576 | I-598 | I-600 | I-616 | I-619 |
| I-620 | I-622 | I-640 | I-641 | I-642 |
| I-643 | I-644 | I-659 | I-660 | I-661 |
| I-665 | I-678 | I-679 | I-680 | I-681 |
| I-682 | I-683 | I-684 | I-685 | I-686 |
| I-688 | I-744 | I-747 | I-755 | I-756 |
| I-758 | I-760 | I-761 | I-766 | I-771 |
| I-773 | I-778 | I-780 | I-781 | I-782 |
| I-783 | I-784 | I-785 | I-786 | I-788 |
| I-789 | I-792 | I-809 | I-811 | I-812 |
| I-814 | I-816 | I-820 | I-826 | I-827 |
| I-828 | I-829 | I-831 | I-832 | I-833 |
| I-836 | I-839 | I-840 | I-845 | I-864 |
| I-865 | I-866 | I-867 | I-868 | I-869 |
| I-870 | I-871 | I-921 | I-922 | I-923 |
| I-924 | I-375 | I-939 | I-940 | I-947 |
| I-974 | I-975 | I-990 | I-1007 | I-1009 |
| I-1010 | I-1015 | I-1020 | I-1021 | I-1022 |
| I-1023 | I-1024 | I-1027 | I-1042 | I-1043 |
| I-1044 | I-1117 | I-1118 | I-1119 | I-1202 |
| I-1214 | I-1215 | I-1216 | I-1220 | I-1222 |
| I-1223 | I-1224 | I-1225 | I-1259 | I-1260 |
| I-1275 | I-1276 | I-1277 | I-1278 | I-1328 |
| I-1329 | I-1330 | I-1331 | I-1332 | I-1333 |
| I-1334 | I-1335 | I-1343 | I-1363 | I-1493 |
| I-1506 | I-1507 | I-1508 | I-1509 | I-1510 |
| I-1511 | I-1512 | I-1513 | I-1514 | I-1515 |
| I-1516 | I-1640 | I-1641 | I-1642 | I-1685 |
| I-2270 | I-2273 | I-3196 | I-3198 | |

### Example 45

### (S)-N-((3-chloro-2,6-difluorophenyl)(cyclopentyl)methyl)-1H-benzo[d]imidazol-2-amine

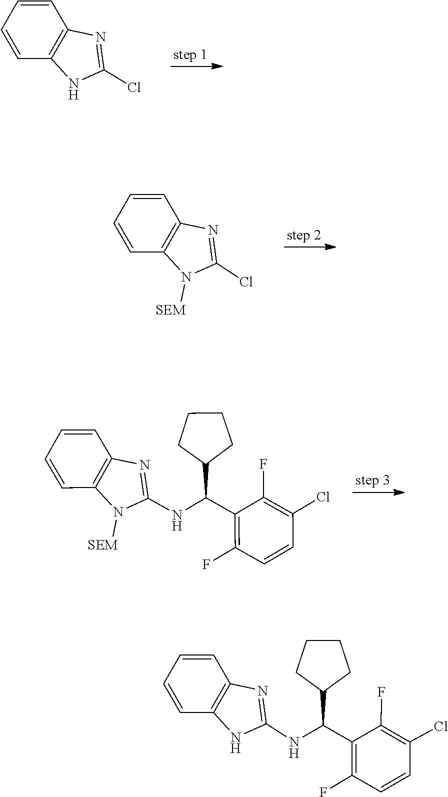 [SMILES: Clc1nc2ccccc2[nH]1 ; C[Si](C)(C)CCOCn1c(Cl)nc2ccccc21 ; C[Si](C)(C)CCOCn1c(N[C@H](c2c(F)ccc(Cl)c2F)C2CCCC2)nc2ccccc21 ; Fc1ccc(Cl)c(F)c1[C@@H](Nc1nc2ccccc2[nH]1)C1CCCC1]

### Step 1. Synthesis of 2-chloro-1-((2-(trimethylsilyl)ethoxy)methyl)-1H-benzo[d]imidazole

To a mixture of 2-chloro-1H-benzo[d]imidazole (800 mg, 5.24 mmol) and Cs₂CO₃ (5.12 g, 15.7 mmol) in DMF (5 mL) was added SEM-Cl (1.39 mL, 7.86 mmol) dropwise at 0° C. under a nitrogen atmosphere. The mixture was stirred for 1 hour at 25° C. The reaction mixture was diluted with water (30 mL), and the aqueous phase was extracted with ethyl acetate (50 mL) three times. The combined organic layers were washed with brine, dried over sodium sulfate, filtered and concentrated in vacuo. The resulting crude material was purified by C18 flash chromatography to afford 2-chloro-1-((2-(trimethylsilyl)ethoxy)methyl)-1H-benzo[d]imidazole (800 mg, 2.83 mmol) as an off-white solid. LCMS RT 0.987 min, [M+H]⁺ 283, LCMS method C.

### Step 2. Synthesis of (S)—N-((3-chloro-2,6-difluorophenyl)(cyclopentyl)methyl)-1-((2-(trimethylsilyl)ethoxy)methyl)-1H-benzo[d]imidazol-2-amine

A mixture of 2-chloro-1-((2-(trimethylsilyl)ethoxy)methyl)-1H-benzo[d]imidazole (300 mg, 1.06 mmol), (S)-(3-chloro-2,6-difluorophenyl)(cyclopentyl)methanamine (261 mg, 1.06 mmol), Cs₂CO₃ (1.04 g, 3.18 mmol), BINAP (66.0 mg, 106 μmol) and Pd₂(dba)₃ (110 mg, 106 μmol) in dioxane (3 mL) was stirred for 16 hours at 110° C. under a N₂ atmosphere. The reaction mixture was diluted with water (20 ml) and extracted with ethyl acetate (50 ml*3). The combined organic layers were washed with brine (10 ml), dried over sodium sulfate, filtered and concentrated in vacuo. The resulting crude material was purified by C18 flash chromatography (CH₃CN/H₂O) to afford (S)—N-((3-chloro-2,6-difluorophenyl)(cyclopentyl) methyl)-1-((2-(trimethylsilyl)ethoxy)methyl)-1H-benzo[d]imidazol-2-amine (150 mg, 305 μmol) as a yellow oil. LCMS RT 1.604 min, [M+H]⁺ 492, LCMS method B.

### Step 3. Synthesis of (S)—N-((3-chloro-2,6-difluorophenyl)(cyclopentyl)methyl)-1H-benzo[d]imidazol-2-amine

A mixture of (S)—N-((3-chloro-2,6-difluorophenyl)(cyclopentyl)methyl)-1-((2-(trimethylsilyl)ethoxy)methyl)-1H-benzo[d]imidazol-2-amine (150 mg, 305 μmol) in TFA (2 mL) was stirred for 1 hour at 25° C. The reaction mixture was concentrated in vacuo. The resulting crude material was purified by preparative HPLC (column: XBridge Prep OBD C18 Column, 30*150 mm, 5 μm; mobile phase A: water (10 mM NH₄HCO₃), mobile phase B: acetonitrile; flow rate: 60 mL/min; gradient: 40% B to 70% B in 8 min, then 70% B; wavelength: 220 nm; RT (min): 7.83). Concentration in vacuo gave (S)—N-((3-chloro-2,6-difluorophenyl)(cyclopentyl)methyl)-1H-benzo[d]imidazol-2-amine (20 mg, 55 μmol) as an off-white amorphous solid. ¹H NMR (400 MHz, DMSO-d₆) δ 10.31 (s, 1H), 7.53 (td, J=8.6, 5.4 Hz, 1H), 7.20-7.09 (m, 3H), 7.07 (d, J=8.9 Hz, 1H), 6.84 (s, 2H), 5.13 (t, J=9.7 Hz, 1H), 2.51 (s, 1H), 1.96-1.88 (m, 1H), 1.64 (s, 2H), 1.57 (dt, J=15.2, 8.1 Hz, 2H), 1.44 (td, J=12.5, 6.6 Hz, 2H), 1.17-1.09 (m, 1H). LCMS RT 0.815 min, [M+H]⁺ 362.05, LCMS method C.

Additional compounds prepared according to the methods of Example 45 are listed in Table 10 below. Corresponding ¹H NMR and mass spectrometry characterization for these compounds are described in Table 1. Certain compounds in Table 10 below were prepared with other compounds whose preparation is described further below in the Examples.

TABLE 10
Additional exemplary compounds

| | | | | |
|---|---|---|---|---|
| I-183 | I-208 | I-224 | I-226 | I-227 |
| I-228 | I-229 | I-234 | I-236 | I-237 |
| I-238 | I-239 | I-240 | I-241 | I-242 |
| I-243 | I-244 | I-245 | I-246 | I-249 |
| I-250 | I-251 | I-269 | I-270 | I-271 |
| I-294 | I-295 | I-296 | I-373 | I-493 |
| I-515 | I-521 | I-522 | I-525 | I-552 |
| I-553 | I-554 | I-555 | I-556 | I-557 |
| I-558 | I-563 | I-564 | I-566 | I-567 |
| I-568 | I-571 | I-572 | I-573 | I-574 |
| I-575 | I-577 | I-578 | I-579 | I-580 |
| I-581 | I-582 | I-583 | I-606 | I-607 |
| I-608 | I-609 | I-610 | I-611 | I-612 |
| I-613 | I-614 | I-615 | I-639 | I-650 |
| I-651 | I-652 | I-657 | I-658 | I-667 |
| I-668 | I-669 | I-671 | I-672 | I-673 |
| I-674 | I-675 | I-676 | I-689 | I-690 |
| I-745 | I-746 | I-748 | I-757 | I-762 |
| I-763 | I-764 | I-765 | I-767 | I-768 |
| I-769 | I-770 | I-772 | I-774 | I-775 |
| I-776 | I-777 | I-787 | I-790 | I-791 |
| I-793 | I-795 | I-796 | I-797 | I-798 |
| I-799 | I-800 | I-801 | I-818 | I-819 |
| I-821 | I-822 | I-824 | I-834 | I-835 |
| I-837 | I-853 | I-854 | I-855 | I-856 |
| I-857 | I-873 | I-874 | I-875 | I-876 |
| I-920 | I-938 | I-976 | I-977 | I-978 |
| I-979 | I-491 | I-1651 | I-1872 | |

### Example 46

### (2r,4r)-N-(4-chloro-1-cyclopentyl-2,3-dihydro-1H-inden-1-yl)-6,8-dioxo-5,7-diazaspiro[3.4]octane-2-carboxamide

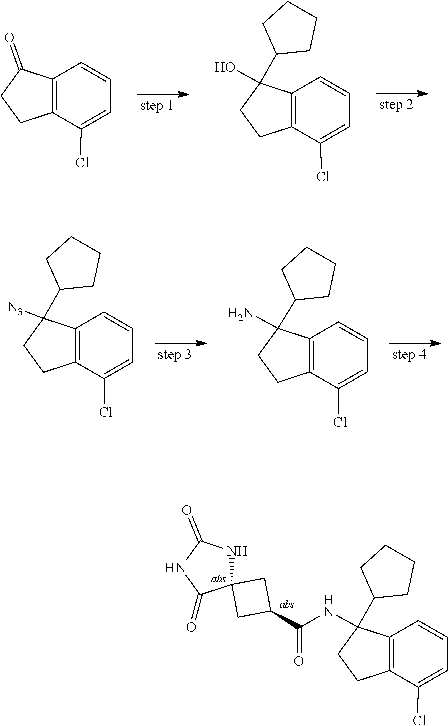 [SMILES: O=C1CCc2c(Cl)cccc21 ; OC1(C2CCCC2)CCc2c(Cl)cccc21 ; [N-]=[N+]=NC1(C2CCCC2)CCc2c(Cl)cccc21 ; NC1(C2CCCC2)CCc2c(Cl)cccc21 ; O=C1NC(=O)[C@]2(C[C@@H](C(=O)NC3(C4CCCC4)CCc4c(Cl)cccc43)C2)N1]

### Step 1. 4-chloro-1-cyclopentyl-2,3-dihydro-1H-inden-1-ol

A flame-dried round-bottomed flask equipped with a magnetic stirrer bar and capped with a rubber septum was charged with a solution of 4-chloro-2,3-dihydro-1H-inden-1-one (83 mg, 0.50 mmol) in THF (1.00 mL). This solution was added dropwise to a separate flame-dried round-bottomed flask containing a stirring solution of LaCl₃ 2LiCl (0.83 mL, 0.6 M in THF, 0.50 mmol) at ambient temperature. The resulting mixture was stirred at ambient temperature for 1 hour, then cooled to 0° C. with stirring. A solution of cyclopentylmagnesium bromide (0.28 mL, 2.0 M in Et₂O, 0.55 mmol) was then added dropwise, and the reaction mixture was stirred at 0° C. for ca. 45 minutes. An additional portion of cyclopentylmagnesium bromide (0.14 mL, 2.0 M in Et₂O, 0.28 mmol) was added dropwise to the reaction mixture after this time, and the mixture was stirred at 0° C. for a further ca. 30 minutes. The reaction was then quenched at 0° C. by slow dropwise addition of saturated aqueous NH₄Cl solution (0.5 mL). Water (0.5 mL) was added to dissolve the precipitated inorganic salts, and the mixture was warmed to ambient temperature with vigorous stirring. The mixture was further diluted with water (20 mL) and the organics were extracted with diethyl ether (3×10 mL). The combined organics were washed with saturated aqueous NaCl solution and dried over MgSO₄, filtered and concentrated in vacuo to give the crude product. Purification by flash chromatography on silica gel (eluent: EtOAc in hexanes, 0:1 to 20:80) afforded 4-chloro-1-cyclopentyl-2,3-dihydro-1H-inden-1-ol (68 mg, 0.29 mmol) as a viscous colorless oil. LCMS RT 1.43 min, (M-OH)⁺ 219.1, LCMS method K. ¹H NMR (400 MHz, CDCl₃) δ 7.25-7.22 (m, 2H), 7.20-7.15 (m, 1H), 3.04 (ddd, J=16.8, 9.0, 4.5 Hz, 1H), 2.84 (ddd, J=16.5, 8.3, 6.5 Hz, 1H), 2.42-2.34 (m, 2H), 2.06 (ddd, J=13.5, 9.0, 6.5 Hz, 1H), 1.83-1.76 (m, 2H), 1.70-1.63 (m, 1H), 1.62-1.49 (m, 5H), 1.32-1.22 (m, 1H).

### Step 2. 1-azido-4-chloro-1-cyclopentyl-2,3-dihydro-1H-indene

A flame-dried round-bottomed flask equipped with a magnetic stirrer bar and capped with a rubber septum was charged with a solution of 4-chloro-1-cyclopentyl-2,3-dihydro-1H-inden-1-ol (58 mg, 0.24 mmol) in anhydrous chloroform (0.70 mL), and the solution was cooled to 0° C. with stirring. To the cooled solution was added solid sodium azide (32 mg, 0.49 mmol) in small portions, followed by slow dropwise addition of trifluoroacetic acid (0.12 mL, 1.60 mmol). The reaction mixture was then warmed to 30° C. with stirring for ca. 2 h. The reaction mixture was then cooled to ambient temperature and carefully quenched under nitrogen with a 10% aqueous solution of NH₄OH until the pH was approximately equal to 8-9. The mixture was then poured into a separatory funnel and extracted with chloroform (3×10 mL). The combined organics were then dried over MgSO₄, filtered and concentrated in vacuo to afford crude 1-azido-4-chloro-1-cyclopentyl-2,3-dihydro-1H-indene, which was utilized immediately in the next step assuming quantitative yield.

### Step 3. 4-chloro-1-cyclopentyl-2,3-dihydro-1H-inden-1-amine

The crude azide was dissolved in THF (2.40 mL) with stirring, and a solution of trimethylphosphine (0.26 mL, 1.0 M in THF, 0.26 mmol) was added dropwise at ambient temperature, followed by dropwise addition of water (0.24 mL). The reaction mixture was then heated to 30° C. with stirring for ca. 18 h. The reaction mixture was then cooled to ambient temperature and diluted with EtOAc (10 mL). The phases were separated, and the organic phase was washed with saturated aqueous NaHCO₃ solution (3×5 mL) and saturated aqueous NaCl solution. The organics were then dried over MgSO₄, filtered and concentrated in vacuo to afford crude 4-chloro-1-cyclopentyl-2,3-dihydro-1H-inden-1-amine, which was utilized immediately in the next step assuming quantitative yield. LCMS RT 0.89 min, [M-NH₂]⁺ 219.2, LCMS method K.

### Step 4. (2r,4r)-N-(4-chloro-1-cyclopentyl-2,3-dihydro-1H-inden-1-yl)-6,8-dioxo-5,7-diazaspiro[3.4]octane-2-carboxamide

The crude amine was dissolved in DMF (2.40 mL) in a round-bottomed flask equipped with a magnetic stirbar at ambient temperature, and (2r,4r)-6,8-dioxo-5,7-diazaspiro[3.4]octane-2-carboxylic acid (47 mg, 0.25 mmol) was added in one portion with stirring. To the mixture were then added dropwise DIPEA (0.17 mL, 0.98 mmol) and a solution of T3P (0.14 mL, 50 wt. % in EtOAc, 0.24 mmol) at ambient temperature, and the reaction mixture was stirred for ca. 1 h. An additional portion of T3P (0.07 mL, 50 wt. % in EtOAc, 0.12 mmol) was added after this time, and the mixture was stirred at ambient temperature for a further ca. 30 mins. The reaction mixture was then diluted with DCM (10 mL), quenched with saturated aqueous NaHCO₃ solution (10 mL), and stirred at ambient temperature overnight. The phases were then separated and the aqueous phase was extracted with DCM (3×10 mL). The combined organics were washed with water (10 ml) and saturated aqueous NaCl solution, dried over MgSO₄, filtered and concentrated in vacuo. The residue was then dissolved in a minimum volume of DMF, loaded onto a 12 g C18 cartridge, and purified by reverse-phase chromatography (mobile phase A: 10 mM ammonium formate in water, mobile phase B: acetonitrile; gradient: 40 to 60% B) to afford (2r,4r)-N-(4-chloro-1-cyclopentyl-2,3-dihydro-1H-inden-1-yl)-6,8-dioxo-5,7-diazaspiro[3.4]octane-2-carboxamide (4 mg) as an amorphous bright yellow solid. LCMS RT 1.21 min, [M+H]⁺ 402.3, LCMS method K. ¹H NMR (400 MHz, DMSO-d6) δ 10.55 (br. s, 1H), 8.61 (s, 1H), 7.86 (s, 1H), 7.24-7.19 (m, 1H), 7.19-7.15 (m, 2H), 3.11 (p, J=9.1 Hz, 1H), 2.99 (ddd, J=16.1, 9.6, 4.8 Hz, 1H), 2.79 (ddd, J=16.5, 9.3, 5.7 Hz, 1H), 2.71-2.43 (overlapping m, 3H), 2.36 (ddd, J=11.6, 8.8, 4.6 Hz, 1H), 2.17-2.06 (m, 3H), 1.80-1.71 (m, 1H), 1.57-1.37 (m, 4H), 1.32-1.13 (m, 2H), 1.08-0.98 (m, 1H).

### Example 47

### 7-(4-(3-chlorophenyl)-4-cyclopentyl-2-oxotetrahydropyrimidin-1(2H)-yl)imidazo[1,5-a]pyridine-3-carboxamide

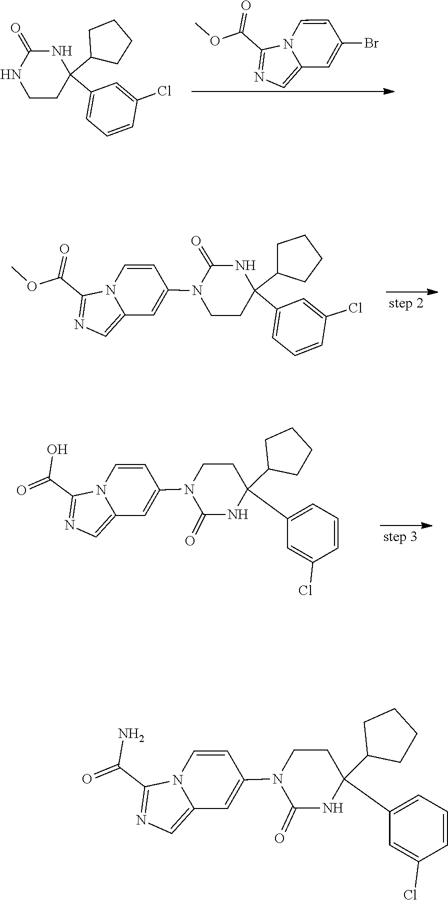 [SMILES: O=C1NCCC(c2cccc(Cl)c2)(C2CCCC2)N1 ; COC(=O)c1ncc2cc(Br)ccn12 ; COC(=O)c1ncc2cc(N3CCC(c4cccc(Cl)c4)(C4CCCC4)NC3=O)ccn12 ; O=C(O)c1ncc2cc(N3CCC(c4cccc(Cl)c4)(C4CCCC4)NC3=O)ccn12 ; NC(=O)c1ncc2cc(N3CCC(c4cccc(Cl)c4)(C4CCCC4)NC3=O)ccn12]

### Step 1. Synthesis of methyl 7-(4-(3-chlorophenyl)-4-cyclopentyl-2-oxotetrahydropyrimidin-1(2H)-yl)imidazo[1,5-a]pyridine-3-carboxylate

A round bottomed flask was charged with 4-(3-chlorophenyl)-4-cyclopentyltetrahydropyrimidin-2(1H)-one (100 mg, 359 μmol), methyl 7-bromoimidazo[1,5-a]pyridine-3-carboxylate (91.5 mg, 359 μmol), Pd-PEPPSI-IPentCl (105 mg, 108 μmol), Cs₂CO₃ (351 mg, 1.08 mmol) and a stirbar. 1,4-Dioxane (1 mL) was added, and the solution was stirred for 4 hours at 90° C. The residue was purified by reverse phase flash chromatography (column: C18 silica gel; mobile phase A: water, mobile phase B: acetonitrile; gradient: 0% to 100% in 10 minutes; detector: UV 220 nm) to give methyl 7-(4-(3-chlorophenyl)-4-cyclopentyl-2-oxotetrahydropyrimidin-1(2H)-yl)imidazo[1,5-a]pyridine-3-carboxylate (30 mg, 66 μmol) as a yellow amorphous solid. LCMS RT 0.988 min, [M+H]453.20, LCMS method C.

### Step 2. Synthesis of 7-(4-(3-chlorophenyl)-4-cyclopentyl-2-oxotetrahydropyrimidin-1(2H)-yl)imidazo[1,5-a]pyridine-3-carboxylic acid

A round bottomed flask was charged with methyl 7-(4-(3-chlorophenyl)-4-cyclopentyl-2-oxotetrahydropyrimidin-1(2H)-yl)imidazo[1,5-a]pyridine-3-carboxylate (30 mg, 66 μmol), NaOH (0.33 mL, 2 molar, 0.66 mmol) and a stirbar. MeOH (1 mL) was added, and the solution was stirred for 1 hour at 25° C. The residue was purified by reverse phase flash chromatography: (column: C18 silica gel; mobile phase A: water, mobile phase B: acetonitrile; gradient: 0% to 100% in 10 minutes; detector: UV 220 nm) to give 7-(4-(3-chlorophenyl)-4-cyclopentyl-2-oxotetrahydropyrimidin-1(2H)-yl)imidazo[1,5-a]pyridine-3-carboxylic acid (25 mg, 57 μmol) as a yellow amorphous solid, which was used in the next step without purification.

### Step 3. Synthesis of 7-(4-(3-chlorophenyl)-4-cyclopentyl-2-oxotetrahydropyrimidin-1(2H)-yl)imidazo[1,5-a]pyridine-3-carboxamide

A round bottomed flask was charged with 7-(4-(3-chlorophenyl)-4-cyclopentyl-2-oxotetrahydropyrimidin-1(2H)-yl)imidazo[1,5-a]pyridine-3-carboxylic acid (25 mg, 57 μmol), NH₄Cl (3.0 mg, 57 μmol), HATU (32 mg, 85 μmol), NaHCO₃ (14 mg, 0.17 mmol) and a stirbar. DMF (1 mL) was added, and the solution was stirred for 1 hour at 25° C. The resulting crude material was purified by chiral Pre-HPLC (Column: (R, R) WHELK-01, 4.6*50 mm, 3.5 μm; Mobile Phase A: Hex (0.2% IPAmine):EtOH=80:20; Flow rate: 1 mL/min; Gradient: 0% B to 0% B; Injection Volume: 5 ul mL). Lyophilization yielded 7-(4-(3-chlorophenyl)-4-cyclopentyl-2-oxotetrahydropyrimidin-1(2H)-yl)imidazo[1,5-a]pyridine-3-carboxamide (7.8 mg, 18 μmol, 31%) as an off-white amorphous solid. LCMS RT 0.903 min, [M+H]⁺ 438.15, LCMS method C.

### Example 48

### (S)—N-((1S,3S)-3-acetamidocyclopentyl)-2-(3-chloro-2,6-difluorophenyl)-2-(4-fluorobicyclo[2.2.1]heptan-1-yl)acetamide

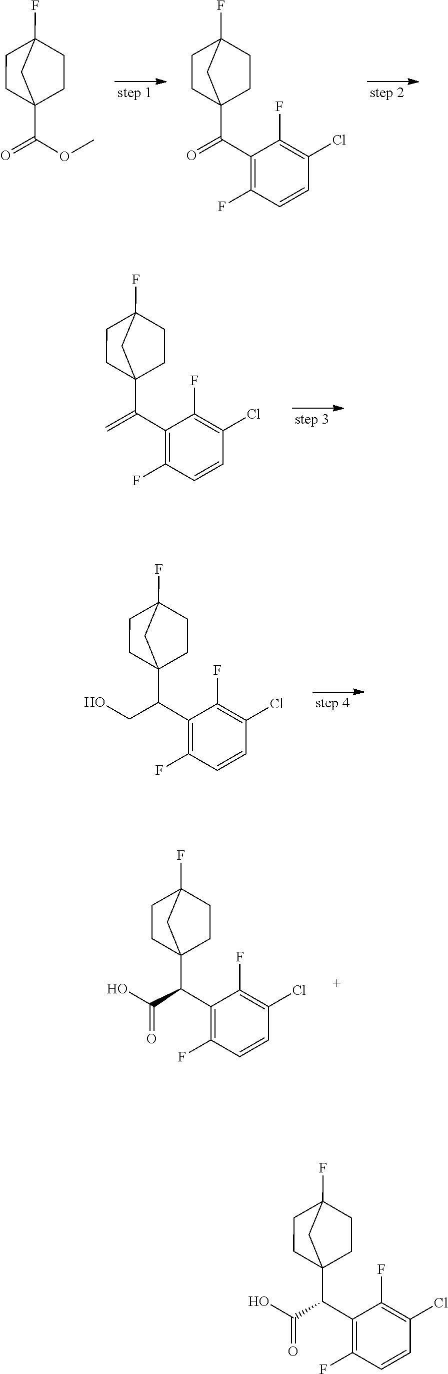 [SMILES: COC(=O)C12CCC(F)(CC1)C2 ; O=C(c1c(F)ccc(Cl)c1F)C12CCC(F)(CC1)C2 ; C=C(c1c(F)ccc(Cl)c1F)C12CCC(F)(CC1)C2 ; OCC(c1c(F)ccc(Cl)c1F)C12CCC(F)(CC1)C2 ; O=C(O)[C@H](c1c(F)ccc(Cl)c1F)C12CCC(F)(CC1)C2 ; O=C(O)[C@@H](c1c(F)ccc(Cl)c1F)C12CCC(F)(CC1)C2]

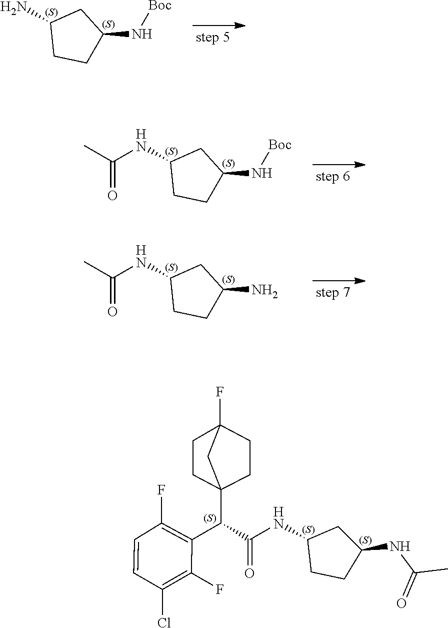

### Step 1. Synthesis of (3-chloro-2,6-difluorophenyl)(4-fluorobicyclo[2.2.1]heptan-1-yl)methanone

n-BuLi (2.5 M, 61.0 mL) was diluted with THF (175 mL). A solution of 1-chloro-2,4-difluorobenzene (18.1 g, 122 mmol) in THF (100 mL) was added dropwise at −78° C. under N₂. After stirring at −78° C. for 2 hours, a solution of methyl 4-fluorobicyclo[2.2.1]heptane-1-carboxylate (17.5 g, 102 mmol) in THF (175 mL) was added dropwise at −78° C. under N₂. The mixture was stirred at −78° C. for 4 hours. The reaction mixture was poured into sat. NH₄Cl solution (350 mL) and extracted with ethyl acetate (200 mL*2). The combined organic layers were washed with brine (200 mL), dried over Na₂SO₄, filtered and concentrated to give a residue. The residue was purified by column chromatography (silica gel, petroleum ether:ethyl acetate 1:0 to 0:1) to give (3-chloro-2,6-difluorophenyl)(4-fluorobicyclo[2.2.1]heptan-1-yl)methanone as a yellow oil. ¹H NMR: (400 MHz, CDCl₃) 37.43 (dt, J=5.6, 8.6 Hz, 1H), 6.93 (ddd, J=1.6, 7.8, 9.0 Hz, 1H), 2.29-2.15 (m, 2H), 2.06-1.93 (m, 4H), 1.92-1.77 (m, 4H).

### Step 2. Synthesis of 1-(1-(3-chloro-2,6-difluorophenyl)vinyl)-4-fluorobicyclo[2.2.1]heptane

To a solution of Ph₃PMeBr (16.3 g, 45.7 mmol) in THF (66.0 mL) was added t-BuOK (1.0 M, 45.7 mL) at 0° C. The mixture was warmed to 15° C. and stirred at 15° C. for 2 hours. Then a solution of (3-chloro-2,6-difluorophenyl)(4-fluorobicyclo[2.2.1]heptan-1-yl)methanone (6.60 g, 22.9 mmol) in THF (66.0 mL) was added at 0° C. The mixture was stirred at 0° C. for 2 hours, then warmed to 15° C. and stirred at 15° C. for 12 hours. The reaction was quenched by addition of water (6.00 mL) and filtered. The filtrate was concentrated under reduced pressure to give a residue. The residue was purified by column chromatography (silica gel, petroleum ether:ethyl acetate 1:0 to 10:1) to give 1-(1-(3-chloro-2,6-difluorophenyl)vinyl)-4-fluorobicyclo[2.2.1]heptane (5.60 g, 17.3 mmol) as a yellow oil. ¹H NMR: (400 MHz, CDCl₃)

δ 7.31 (dt, J=5.6, 8.4 Hz, 1H), 6.87 (br d, J=0.6 Hz, 1H), 5.43 (s, 1H), 5.06 (s, 1H), 2.02-1.89 (m, 4H), 1.83-1.74 (m, 4H), 1.70-1.61 (m, 2H).

### Step 3. Synthesis of 2-(3-chloro-2,6-difluorophenyl)-2-(4-fluorobicyclo[2.2.1]heptan-1-yl)ethan-1-ol

To a solution of 1-(1-(3-chloro-2,6-difluorophenyl)vinyl)-4-fluorobicyclo[2.2.1]heptane (5.50 g, 19.2 mmol) in THF (165 mL) was added BH₃-Me₂S (3.84 mL) at 25° C. under N₂. The mixture was heated to 50° C. and stirred at 50° C. 1 hour. MeOH (18.7 mL) was added dropwise at 0° C. After that NaOH (2 M, 28.8 mL) was added dropwise at 0° C., then H₂O₂ (30%, 9.28 mL, 96.6 mmol) was added at 0° C. slowly. The mixture was stirred at 0° C. for 1.5 hours. The mixture was poured into sat. Na₂S₂O₃ aqueous solution (200 mL) slowly, stirred for 10 minutes, and extracted with ethyl acetate (200 mL*2). The combined organic phases were washed with water (200 mL), brine (200 mL), dried over Na₂SO₄, filtered and concentrated. The residue was purified by column chromatography (silica gel, petroleum ether:ethyl acetate 1:0 to 0:1) to give 2-(3-chloro-2,6-difluorophenyl)-2-(4-fluorobicyclo[2.2.1]heptan-1-yl)ethan-1-ol (4.80 g, 15.8 mmol) as a yellow oil. ¹H NMR: (400 MHz, DMSO) δ 7.52 (dt, J=5.6, 8.6 Hz, 1H), 7.19-7.05 (m, 1H), 4.72-4.63 (m, 1H), 3.93-3.77 (m, 2H), 3.43-3.35 (m, 1H), 1.88-1.57 (m, 7H), 1.52-1.29 (m, 3H).

### Step 4. Synthesis of (R)-2-(3-chloro-2,6-difluorophenyl)-2-(4-fluorobicyclo[2.2.1]heptan-1-yl)acetic acid and (S)-2-(3-chloro-2,6-difluorophenyl)-2-(4-fluorobicyclo[2.2.1]heptan-1-yl)acetic acid

To a solution of 2-(3-chloro-2,6-difluorophenyl)-2-(4-fluorobicyclo[2.2.1]heptan-1-yl)ethan-1-ol (4.80 g, 15.8 mmol) in acetonitrile (76.0 mL) was added a solution of NaClO₂ (11.4 g, 126 mmol) in H₂O (14.0 mL) at 0° C. Then TEMPO (297 mg, 1.89 mmol), a solution of Na₂HPO₄ (0.67 M, 23.5 mL) and NaH₂PO₄ (0.67 M, 23.5 mL) in water, and a solution of NaClO (2.35 g, 1.89 mmol, 1.94 mL) in H₂O (14.0 mL) was added at 0° C. The mixture was warmed to 15° C. and stirred at 15° C. for 12 hours. The reaction mixture was cooled to 0° C. Water (200 mL) was added, followed by Na₂SO₃ (28.4 g) at 0° C. The mixture was stirred at 15° C. for 30 minutes. The pH was adjusted to 1-2 with H₃PO₄, and the solution was extracted with ethyl acetate (200 mL*2). The combined organic layers were washed with brine (200 mL), dried over Na₂SO₄, filtered and concentrated under reduced pressure. The residue was purified by column chromatography (silica gel, petroleum ether:ethyl acetate 1:0 to 0:1) to give 2-(3-chloro-2,6-difluorophenyl)-2-(4-fluorobicyclo[2.2.1]heptan-1-yl)acetic acid as a white solid. It was further purified by chiral SFC (column: DAICEL CHIRALPAK AS 250 mm*30 mm, 10 μm); mobile phase: [CO₂-ⁱPrOH]; gradient: 15% ⁱPrOH isocratic) to give (R)-2-(3-chloro-2,6-difluorophenyl)-2-(4-fluorobicyclo[2.2.1]heptan-1-yl)acetic acid and (S)-2-(3-chloro-2,6-difluorophenyl)-2-(4-fluorobicyclo[2.2.1]heptan-1-yl)acetic acid.

Isomer 1: 2.00 g, 6.28 mmol was obtained as a white solid. ¹H NMR: (400 MHz, CDCl₃) δ 7.37 (dt, J=5.6, 8.6 Hz, 1H), 6.93 (dt, J=1.6, 9.0 Hz, IH), 4.21 (s, 1H), 2.07-1.91 (m, 3H), 1.91-1.80 (m, 3H), 1.80-1.68 (m, 2H), 1.67-1.56 (m, 2H).

Isomer 2: 2.01 g, 6.28 mmol was obtained as a white solid. ¹H NMR: (400 MHz, CDCl₃) (7.37 (dt, J=5.6, 8.6 Hz, 1H), 6.93 (dt, J=1.6, 9.0 Hz, IH), 4.20 (s, 1H), 2.07-1.93 (m, 3H), 1.92-1.81 (m, 3H), 1.81-1.70 (m, 2H), 1.68-1.57 (m, 2H).

### Step 5. Synthesis of tert-butyl ((1S,3S)-3-acetamidocyclopentyl) carbamate

To a mixture of tert-butyl ((1S,3S)-3-aminocyclopentyl) carbamate (500 mg, 2.50 m mol) and TEA (1.04 mL, 7.49 mmol) in DCM (8 mL) was added Ac₂O (283 μL, 3.00 mmol) dropwise at 0° C. under a nitrogen atmosphere. The mixture was stirred for 2 hours at room temperature. The mixture was concentrated. The resulting crude material was purified by reverse phase flash chromatography (column: C18 silica gel; mobile phase A: water, mobile phase B: acetonitrile; gradient: 0% to 100% B in 20 min; detector: UV 200 nm) to give tert-butyl ((1S,3S)-3-acetamidocyclopentyl) carbamate (300 mg, 1.24 mmol) as an off-white solid. LCMS RT 0.738 min, [M+H]⁺ 243.15, LCMS method B.

### Step 6. Synthesis of N-((1S,3S)-3-aminocyclopentyl) acetamide

A mixture of tert-butyl ((1S,3S)-3-acetamidocyclopentyl) carbamate (120 mg, 495 μmol) in DCM:TFA (2:1, 1 mL) was stirred for 2 hours at room temperature. The mixture w as concentrated in vacuo to give N-((1S,3S)-3-aminocyclopentyl)acetamide (60 mg, 0.42 m molas a colorless oil. LCMS RT 0.158 min, [M+H]⁻ 142.00, LCMS method B.

### Step 7. Synthesis of (S)—N-((1S,3S)-3-acetamidocyclopentyl)-2-(3-chloro-2,6-difluorophenyl)-2-(4-fluorobicyclo [2.2.1]heptan-1-yl) acetamide

To a mixture of (S)-2-(3-chloro-2,6-difluorophenyl)-2-(4-fluorobicyclo [2.2.1]heptan-1-yl) acetic acid (25 mg, 78 μmol), N-((1S,3S)-3-aminocyclopentyl) acetamide (13 mg, 94 μmol) and NaHCO₃ (33 mg, 0.39 mmol) in DMF (1 mL) was added HATU (60 mg, 0.16 mmol). The mixture was stirred for 6 hours at 25° C. The reaction mixture was diluted with water (10 mL), and the aqueous phase was extracted with ethyl acetate (20 mL) three times. The combined organic layers were washed with brine, dried over sodium sulfate, filtered and concentrated in vacuo. The resulting crude material was purified by preparative HPLC (column: XBridge Prep OBD C18 Column, 30*150 mm, 5 μm; mobile phase A: water (10 mM NH₄HCO₃)+0.05% NH₄OH, mobile phase B: acetonitrile; flow rate: 60 mL/min; gradient: 31% B to 58% B in 7 min; wavelength: 254/220 nm; RT (min): 7.62) to give (S)—N-((1S,3S)-3-acetamidocyclopentyl)-2-(3-chloro-2,6-difluorophenyl)-2-(4-fluorobicyclo[2.2.1]heptan-1-yl)acetamide (3.6 mg, 8.1 mol) as an off-white amorphous solid. ¹HNMR (400 MHz, DMSO-d6) δ 7.83 (s, 1H), 7.69 (d, J=7.3 Hz, 1H), 7.58 (td, J=8.7, 5.5 Hz, 1H), 7.15 (td, J=9.3, 1.6 Hz, 1H), 4.13 (p, J=7.1 Hz, 1H), 3.97 (p, J=7.0 Hz, 1H), 3.86 (s, 1H), 1.94-1.76 (m, 5H), 1.70 (d, J=28.3 Hz, 9H), 1.62-1.38 (m, 3H), 1.38-1.14 (m, 2H). LCMS RT 1.008 min, [M+H]⁺ 443.25, LCMS method D.

Additional compounds prepared according to the methods of Example 48 are listed in Table 11 below. Corresponding ¹H NMR and mass spectrometry characterization for these compounds are described in Table 1. Certain compounds in Table 11 below were prepared with other compounds whose preparation is described further below in the Examples.

TABLE 11
Additional exemplary compounds

| | | | | |
|---|---|---|---|---|
| I-272 | I-273 | I-297 | I-298 | I-3720 |
| I-3722 | | | | |

### Example 49

### (2r,4S)—N—((S)-2-amino-1-(3-chlorophenyl)-1-cyclopentyl-2-oxoethyl)-6,8-dioxo-5,7-diazaspiro[3.4]octane-2-carboxamide

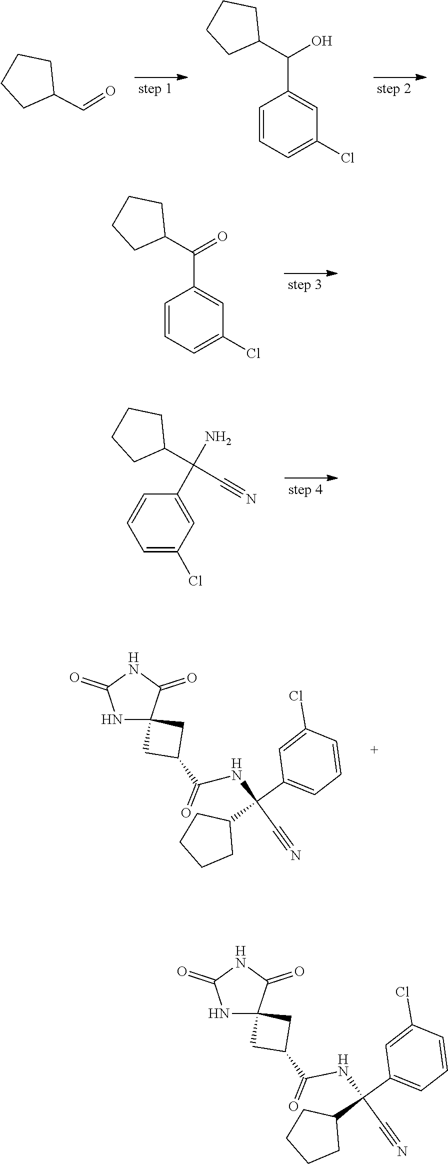 [SMILES: O=CC1CCCC1 ; OC(c1cccc(Cl)c1)C1CCCC1 ; O=C(c1cccc(Cl)c1)C1CCCC1 ; N#CC(N)(c1cccc(Cl)c1)C1CCCC1 ; N#C[C@@](NC(=O)[C@H]1C[C@@]2(C1)NC(=O)NC2=O)(c1cccc(Cl)c1)C1CCCC1 ; N#C[C@](NC(=O)[C@H]1C[C@@]2(C1)NC(=O)NC2=O)(c1cccc(Cl)c1)C1CCCC1]

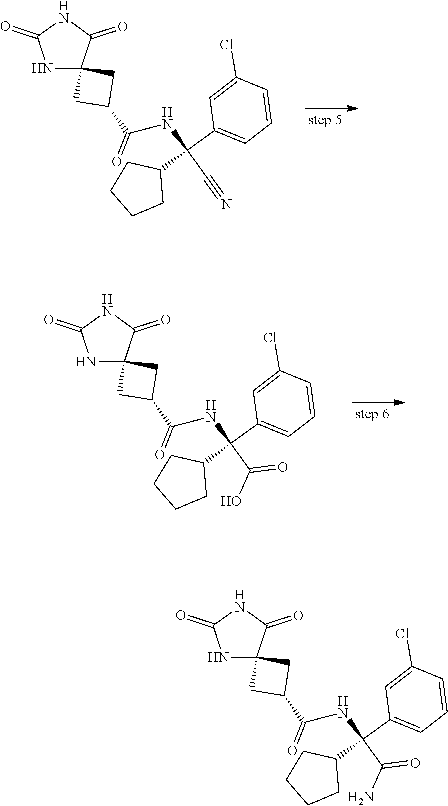 [SMILES: N#C[C@@](NC(=O)[C@H]1C[C@@]2(C1)NC(=O)NC2=O)(c1cccc(Cl)c1)C1CCCC1 ; O=C1NC(=O)[C@]2(C[C@@H](C(=O)N[C@](C(=O)O)(c3cccc(Cl)c3)C3CCCC3)C2)N1 ; NC(=O)[C@@](NC(=O)[C@H]1C[C@@]2(C1)NC(=O)NC2=O)(c1cccc(Cl)c1)C1CCCC1]

### Step 1. Synthesis of (3-chlorophenyl)(cyclopentyl)methanol

To a mixture of cyclopentanecarbaldehyde (3.92 g, 0.040 mol) in THF (30 mL) was added (3-chlorophenyl)magnesium bromide (i M in THF, 40 ml, 0.040 mol) dropwise at −78° C. under a nitrogen atmosphere. The mixture was stirred for 2 hours at −78° C. The reaction was quenched with saturated NH₄Cl (aq.) and the aqueous phase was extracted with ethyl acetate (100 mL) three times. The combined organic layers were washed with brine, dried over sodium sulfate, filtered and concentrated in vacuo. The residue was purified by C18 flash chromatography (CH₃CN/water) to afford (3-chlorophenyl)(cyclopentyl)methanol (2.74 g, 0.013 mol, 30%) as a yellow oil. LCMS RT 1.003 min, [M+H]⁺ not observed, LCMS method C.

### Step 2. Synthesis of (3-chlorophenyl)(cyclopentyl)methanone

To a mixture of (3-chlorophenyl)(cyclopentyl)methanol (1.05 g, 5.0 mmol) and molecular sieve 4 Å (5.0 g) in DCM (10 mL) was added PCC (1.29 g, 6.0 mmol) in portions at 0° C. under a nitrogen atmosphere. The mixture was stirred for 2 hours at 25° C. The reaction mixture was filtered through Celite, the pad was washed with DCM, and the filtrate was concentrated in vacuo to give (3-chlorophenyl)(cyclopentyl)methanone (1.22 g, 5.85 mmol) as a yellow oil. ¹H NMR 17 (400 MHz, DMSO-d₆) δ 7.93 (dt, J=6.0, 1.6 Hz, 2H), 7.76-7.65 (m, 1H), 7.56 (t, J=8.1 Hz, 1H), 3.83 (tt, J=8.8, 6.8 Hz, 1H), 1.88 (ddt, J=12.7, 8.8, 6.4 Hz, 2H), 1.78-1.66 (m, 2H), 1.70-1.54 (m, 4H).

### Step 3. Synthesis of 2-amino-2-(3-chlorophenyl)-2-cyclopentylacetonitrile

A mixture of (3-chlorophenyl)(cyclopentyl)methanone (1.04 g, 5.0 mmol), TMSCN (1.98 g, 0.02 mol) and NH₃ (10 mL, 7 N in MeOH) was stirred for 16 hours at 90° C. The reaction mixture was concentrated in vacuo. The residue was purified by C18 flash chromatography (CH₃CN/H₂O) to afford 2-amino-2-(3-chlorophenyl)-2-cyclopentylacetonitrile (600 mg, 2.56 mmol) as a yellow oil. LCMS RT 0.870 min, [M+H]⁺ 235, LCMS method C.

### Step 4. Synthesis of (2r,4S)—N—((S)-(3-chlorophenyl)(cyano)(cyclopentyl)methyl)-6,8-dioxo-5,7-diazaspiro[3.4]octane-2-carboxamide and (2r,4R)—N—((R)-(3-chlorophenyl)(cyano)(cyclopentyl)methyl)-6,8-dioxo-5,7-diazaspiro[3.4]octane-2-carboxamide

A mixture of 2-amino-2-(3-chlorophenyl)-2-cyclopentylacetonitrile (500 mg, 2.13 m mol), (2r,4r)-6,8-dioxo-5,7-diazaspiro[3.4]octane-2-carboxylic acid (392 mg, 2.13 mmol), T EA (891 μL, 6.39 mmol) and T₃P (1.02 g, 3.20 mmol) in DMF (5 mL) was stirred for 1 hour at room temperature. The reaction mixture was diluted with water (30 mL), and the aqueous phase was extracted with ethyl acetate (50 mL) three times. The combined organic layers were washed with brine, dried over sodium sulfate, filtered and concentrated in vacuo. The resulting crude material was purified by C18 flash chromatography to afford (2r,4r)-N-((3-chlorophenyl)(cyano)(cyclopentyl)methyl)-6,8-dioxo-5,7-diazaspiro[3.4]octane-2-carboxamide as a white solid. LCMS RT 0.855 min, [M+H]⁺ 401, LCMS method D.

The product was further purified by chiral preparative HPLC (column: DZ-CHIRALPAK IH-3, 4.6*50 mm, 3.0 μm; mobile phase A: hexane; mobile phase B: EtOH; flow rate: 1 mL/min; gradient: 20% B isocratic; injection volume: 5 mL) to give (2r,4S)—N—((S)-(3-chlorophenyl)(cyano)(cyclopentyl)methyl)-6,8-dioxo-5,7-diazaspiro[3.4]octane-2-carboxamide and (2r,4R)—N—((R)-(3-chlorophenyl)(cyano)(cyclopentyl)methyl)-6,8-dioxo-5,7-diazaspiro[3.4]octane-2-carboxamide, both as an off-white amorphous solid.

Isomer 1:10 mg. ¹H NMR (400 MHz, DMSO-d₆) δ 10.59 (s, 1H), 8.99 (s, 1H), 8.64 (s, 1H), 7.50-7.38 (m, 2H), 7.35 (dt, J=4.6, 1.9 Hz, 2H), 3.34 (s, 1H), 2.68 (t, J=10.9 Hz, 1H), 2.44 (t, J=8.6 Hz, 1H), 2.22 (dd, J=12.8, 9.0 Hz, 2H), 2.05 (dd, J=13.4, 8.0 Hz, 1H), 1.62-1.48 (m, 4H), 1.43-1.13 (m, 4H). LCMS RT 0.838 min, [M+H]⁺ 401.10, LCMS method C.

Isomer 2: 5 mg. LCMS 1.318 min, [M+H]⁺ 401.10, LCMS method B. ¹H NMR (400 MHz, DMSO-d₆) δ 10.60 (s, 1H), 8098 (s, 1H), 8.64 (s, 1H), 7.34-7.47 (m, 4H), 3.24 (t, J=8.8 Hz, 1H), 2.66-2.69 (m, 1H), 2.40-2.58 (m, 2H), 2.19-2.30 (m, 2H), 2.01-2.08 (m, 1H), 1.40-1.72 (m, 5H), 1.10-1.29 (m, 2H).

### Step 5. Synthesis of (S)-2-(3-chlorophenyl)-2-cyclopentyl-2-((2r,4S)-6,8-dioxo-5,7-diazaspiro[3.4]octane-2-carboxamido)acetic acid

A mixture of (2r,4S)—N—((S)-(3-chlorophenyl)(cyano)(cyclopentyl)methyl)-6,8-dioxo-5,7-diazaspiro[3.4]octane-2-carboxamide (85 mg, 0.21 mmol) and HCl (5 mL, 12 N) was stirred for 1 h at 40° C. After cooling to room temperature, the reaction mixture was extracted with dichloromethane (20 mL) three times. The combined organic layers were washed with brine, dried over sodium sulfate, filtered and concentrated in vacuo to afford (S)-2-(3-chlorophenyl)-2-cyclopentyl-2-((2r,4S)-6,8-dioxo-5,7-diazaspiro[3.4]octane-2-carboxamido)acetic acid (70 mg, 0.17 mmol) as a colorless oil. LCMS RT 0.820 min, [M+H]⁺ 420.0, LCMS method D.

### Step 6. Synthesis of (2r,4S)—N—((S)-2-amino-1-(3-chlorophenyl)-1-cyclopentyl-2-oxoethyl)-6,8-dioxo-5,7-diazaspiro[3.4]octane-2-carboxamide

A mixture of (S)-2-(3-chlorophenyl)-2-cyclopentyl-2-((2r,4S)-6,8-dioxo-5,7-diazaspiro[3.4]octane-2-carboxamido)acetic acid (75 mg, 0.18 mmol), DIEA (93 μL, 0.54 mmol), HATU (0.10 g, 0.27 mmol) and NH₄Cl (10 mg, 0.20 mmol) in DMF (2 mL) was stirred for 1 hour at room temperature. The reaction mixture was diluted with water (10 mL), and the aqueous phase was extracted with ethyl acetate (10 mL) three times. The combined organic layers were washed with brine, dried over sodium sulfate, filtered and concentrated in vacuo. The resulting crude material was purified by C18 flash chromatography (CH₃CN/H₂O) to give (2r,4S)—N—((S)-2-amino-1-(3-chlorophenyl)-1-cyclopentyl-2-oxoethyl)-6,8-dioxo-5,7-diazaspiro[3.4]octane-2-carboxamide (50 mg, 0.12 mmol) as colorless oil. ¹H NMR (400 MHz, DMSO-d₆) 10.59 (s, 1H), 8.58 (s, 1H), 7.95 (s, 1H), 7.54 (s, 1H), 7.37 (s, 1H), 7.28 (d, J=13.4 Hz, 2H), 7.18 (s, 1H), 7.10 (s, 1H), 2.72 (s, 2H), 2.62 (s, 1H), 2.28 (d, J=12.5 Hz, 2H), 1.58 (s, 1H), 1.42 (s, 8H). LCMS RT 0.715 min, [M+H]⁺ 419, LCMS method C.

### Example 50

### (S)-2-(2-(((3-chloro-2,6-difluorophenyl)(cyclopentyl)methyl)amino)phenyl)ethan-1-ol

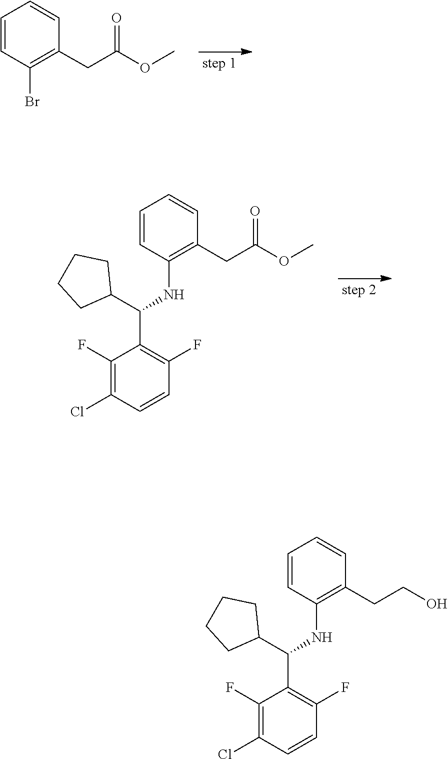 [SMILES: COC(=O)Cc1ccccc1Br ; COC(=O)Cc1ccccc1N[C@H](c1c(F)ccc(Cl)c1F)C1CCCC1 ; OCCc1ccccc1N[C@H](c1c(F)ccc(Cl)c1F)C1CCCC1]

### Step 1. Synthesis of methyl (S)-2-(2-(((3-chloro-2,6-difluorophenyl)(cyclopentyl)methyl)amino)phenyl)acetate

To a mixture of methyl 2-(2-bromophenyl)acetate (400 mg, 1.75 mmol), (S)-(3-chloro-2,6-difluorophenyl)(cyclopentyl)methanamine (429 mg, 1.75 mmol) and Cs₂CO₃ (1.70 g, 5.24 mmol) in toluene (1 mL) was added Pd-PEPPSI-IHept-Cl (CAS: 1814936-54-3) (170 mg, 175 μmol) under a N₂ atmosphere. The mixture was stirred for 16 h at 100° C. After cooling to room temperature, the reaction mixture was diluted with water (50 mL), and the aqueous phase was extracted with ethyl acetate (50 mL*3). The combined organic layers were washed with brine, dried over sodium sulfate, filtered and concentrated in vacuo. The residue was purified by reverse phase flash chromatography (column, C18 silica gel; mobile phase A: water, mobile phase B: acetonitrile; gradient: 0% to 100% in 20 min; detector: UV 220 nm) to give methyl (S)-2-(2-(((3-chloro-2,6-difluorophenyl)(cyclopentyl)methyl)amino)phenyl)acetate (380 mg, 965 μmol) as a yellow oil. LCMS RT 1.530 min, [M+H]⁺ 394.05, LCMS method B.

### Step 2. Synthesis of (S)-2-(2-(((3-chloro-2,6-difluorophenyl)(cyclopentyl)methyl)amino)phenyl)ethan-1-ol

To a mixture of methyl (S)-2-(2-(((3-chloro-2,6-difluorophenyl) (cyclopentyl) methyl) amino) phenyl) acetate (100 mg, 254 μmol) in THF (1 mL) was added LiAlH₄ (19 mg, 508 μmol) in portions at 0° C. The mixture was stirred for 1 h at 25° C. The reaction was quenched with H₂O (19 μl), NaOH (4N, 38 μl), H₂O (19 μl). The mixture was filtered through a pad of Celite, the pad was washed with ethyl acetate, and the combined filtrate was concentrated in vacuo. The resulting crude material was purified by preparative HPLC (column: XBridge Prep Phenyl OBD Column, 19*250 mm, 5 μm; mobile phase A: water (10 mM NH₄HCO₃), mobile phase B: acetonitrile; flow rate: 25 mL/min; gradient: 55% B to 75% B in 10 min; wavelength: 220 nm; RT1 (min): 9.77) to give (S)-2-(2-(((3-chloro-2,6-difluorophenyl)(cyclopentyl)methyl)amino)phenyl)ethan-1-ol (5 mg, 0.01 mmol) as a colorless oil. ¹H NMR (400 MHz, DMSO-d6) δ 7.52 (td, J=8.8, 5.6 Hz, 1H), 7.14 (td, J=9.5, 1.6 Hz, 1H), 6.97-6.91 (m, 2H), 6.50 (td, J=7.4, 1.1 Hz, 1H), 6.41 (d, J=7.9 Hz, 1H), 5.34 (d, J=8.7 Hz, 1H), 4.85 (t, J=5.0 Hz, 1H), 4.49 (t, J=9.5 Hz, 1H), 3.59 (dt, J=7.1, 5.6 Hz, 2H), 2.70-2.55 (m, 3H), 2.13-2.03 (m, 1H), 1.71-1.34 (m, 6H), 1.11 (dq, J=16.2, 8.2, 6.8 Hz, 1H). LCMS RT 1.383 min, [M+H]⁺ 366.10, LCMS method B.

Additional compounds prepared according to the methods of Example 50 are listed in Table 12 below. Corresponding ¹H NMR and mass spectrometry characterization for these compounds are described in Table 1. Certain compounds in Table 12 below were prepared with other compounds whose preparation is described further below in the Examples.

TABLE 12
other exemplary compounds

| | | | | |
|---|---|---|---|---|
| I-1790 | I-1817 | I-1839 | I-1844 | I-1853 |
| I-1969 | | | | |

### Example 51

### (R)-1-(1-(4,6-difluoro-1-methyl-1H-benzo[d]imidazol-2-yl)-2,2,2-trifluoroethyl)-3-(2-(3-hydroxyazetidin-1-yl)pyrimidin-5-yl)urea and (S)-1-(1-(4,6-difluoro-1-methyl-1H-benzo[d]imidazol-2-yl)-2,2,2-trifluoroethyl)-3-(2-(3-hydroxyazetidin-1-yl)pyrimidin-5-yl)urea

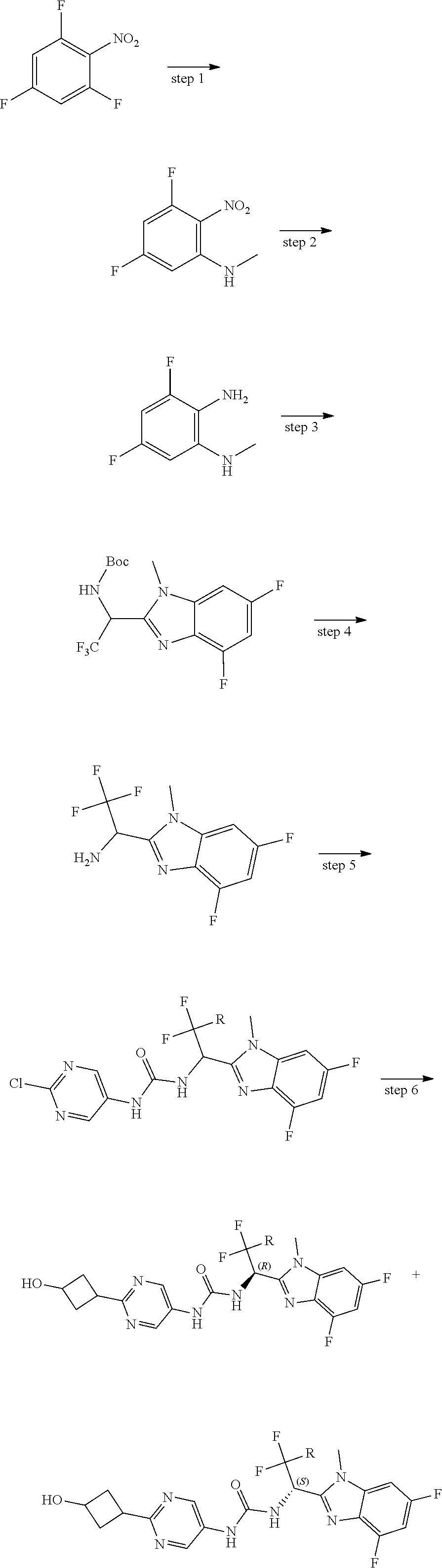

### Step 1. Synthesis of 3,5-difluoro-N-methyl-2-nitroaniline

To a stirred mixture of 1,3,5-trifluoro-2-nitrobenzene (50 mg, 0.28 mmol) and methanamine (13 mg, 0.42 mmol) in THF (1 mL) was added TEA (86 mg, 0.85 mmol) at 25° C. under a nitrogen atmosphere. The resulting mixture was stirred at 25° C. for 16 hours under nitrogen. The mixture was filtered, and the filter cake was washed with EtOAc (3×50 mL). The filtrate was concentrated under reduced pressure. The residue was purified by silica gel column chromatography, eluting with petroleum ether/EtOAc (10:1) to give 3,5-difluoro-N-methyl-2-nitroaniline (30 mg, 0.16 mmol) as a yellow oil. ¹H NMR (300 MHz, DMSO-d6) δ 7.73 (s, 1H), 6.70-6.49 (m, 2H), 2.86 (d, J=4.9 Hz, 3H).

### Step 2. Synthesis of 3,5-difluoro-N1-methylbenzene-1,2-diamine

To a stirred mixture of 3,5-difluoro-N-methyl-2-nitroaniline (200 mg, 1.06 mmol) and Zn powder (695 mg, 10.6 mmol) in MeOH (1 mL) was added saturated NH₄Cl solution (1 mL) at room temperature under a nitrogen atmosphere. The resulting mixture was stirred for 3 hours at 50° C. under nitrogen. After cooling to room temperature, the mixture was filtered, and the filter cake was washed with EtOH (3×50 mL). The filtrate was concentrated under reduced pressure. The residue was diluted with water (100 mL) and extracted with DCM (2×100 mL). The combined organic layers were washed with water (1×100 mL) and brine (1×100 mL), and dried over anhydrous Na₂SO₄. After filtration, the filtrate was concentrated under reduced pressure to afford 3,5-difluoro-N1-methylbenzene-1,2-diamine (120 mg, 759 μmol) as a black solid. LCMS RT 1.087 min, [M−H]⁻ 157.00, LCMS method E. ¹H NMR (400 MHz, DMSO-d6) δ 6.27 (ddd, J=10.9, 9.1, 2.8 Hz, 1H), 6.08 (ddd, J=11.6, 2.8, 1.6 Hz, 1H), 5.32 (s, 1H), 4.19 (s, 2H), 2.72 (d, J=4.9 Hz, 3H).

### Step 3. Synthesis of tert-butyl (1-(4,6-difluoro-1-methyl-1H-benzo[d]imidazol-2-yl)-2,2,2-trifluoroethyl)carbamate

To a stirred mixture of 3,5-difluoro-N1-methylbenzene-1,2-diamine (200 mg, 1.26 mmol) and 2-((tert-butoxycarbonyl)amino)-3,3,3-trifluoropropanoic acid (308 mg, 1.26 mmol) in DMF (1 mL) were added HATU (736 mg, 1.39 mmol) and TEA (647 mg, 2.53 mmol) at room temperature under a nitrogen atmosphere. The resulting mixture was stirred at 60° C. overnight under nitrogen. After cooling to room temperature, water was added and the mixture was extracted with EtOAc (3×100 mL). The combined organic layers were washed with brine (1×100 mL) and dried over anhydrous Na₂SO₄. After filtration, the filtrate was concentrated under reduced pressure. The residue was purified by silica gel column chromatography, eluting with petroleum ether/EtOAc (10:1) to afford tert-butyl (3-((2,4-difluoro-6-(methylamino)phenyl)amino)-1,1,1-trifluoro-3-oxopropan-2-yl)carbamate (150 mg) as a yellow solid.

A solution of tert-butyl (3-((2,4-difluoro-6-(methylamino)phenyl)amino)-1,1,1-trifluoro-3-oxopropan-2-yl)carbamate (140 mg) in HOAc (2 mL) was stirred for 80° C. at 3 hours under a nitrogen atmosphere. The resulting mixture was concentrated under reduced pressure to afford tert-butyl (1-(4,6-difluoro-1-methyl-1H-benzo[d]imidazol-2-yl)-2,2,2-trifluoroethyl)carbamate (150 mg, 0.25 mmol) as a yellow solid, which was used in the next step without purification.

### Step 4. Synthesis of 1-(4,6-difluoro-1-methyl-1H-benzo[d]imidazol-2-yl)-2,2,2-trifluoroethan-1-amine

To a stirred mixture of tert-butyl (1-(4,6-difluoro-1-methyl-1H-benzo[d]imidazol-2-yl)-2,2,2-trifluoroethyl)carbamate (126 mg, 345 μmol) in DCM (1 mL) was added HCl in 1,4-dioxane (2 mL, 1 M, 2 mmol) at 25° C. under a nitrogen atmosphere. The resulting mixture was stirred for 2 hours at 25° C. under nitrogen. The mixture was concentrated under reduced pressure to afford 1-(4,6-difluoro-1-methyl-1H-benzo[d]imidazol-2-yl)-2,2,2-trifluoroethan-1-amine (140 mg, 528 μmol) as a yellow solid. LCMS RT 1.027 min, [M+H]⁺ 265.95, LCMS method E.

### Step 5. 1-(2-chloropyrimidin-5-yl)-3-(1-(4,6-difluoro-1-methyl-1H-benzo[d]imidazol-2-yl)-2,2,2-trifluoroethyl)urea

To a stirred mixture of 1-(4,6-difluoro-1-methyl-1H-benzo[d]imidazol-2-yl)-2,2,2-trifluoroethan-1-amine (155 mg, 584 μmol) in pyridine (2 mL) was added phenyl (2-chloropyrimidin-5-yl)carbamate (146 mg, 584 μmol) at 25° C. under a nitrogen atmosphere. The resulting mixture was stirred for 16 hours at 80° C. under nitrogen. The resulting mixture was extracted with DCM (2×100 mL). The combined organic layers were washed with brine (3×50 mL) and dried over anhydrous Na₂SO₄. After filtration, the filtrate was concentrated under reduced pressure. The residue was purified by silica gel column chromatography, eluted with petroleum ether/EtOAc (10:1) to afford 1-(2-chloropyrimidin-5-yl)-3-(1-(4,6-difluoro-1-methyl-IH-benzo[d]imidazol-2-yl)-2,2,2-trifluoroethyl)urea (80 mg, 0.19 mmol) as a yellow solid. LCMS RT 1.142 min, [M+H]⁻ 421.05, LCMS method E. ¹H NMR (300 MHz, DMSO-d6) δ 9.35 (s, 1H), 8.82 (d, J=6.4 Hz, 2H), 8.10 (d, J=9.0 Hz, 1H), 7.65-7.45 (m, 1H), 7.27-7.11 (m, 1H), 6.28 (p, J=7.1 Hz, 1H), 3.91 (s, 3H).

### Step 6. Synthesis of (R)-1-(1-(4,6-difluoro-1-methyl-1H-benzo[d]imidazol-2-yl)-2,2,2-trifluoroethyl)-3-(2-(3-hydroxyazetidin-1-yl)pyrimidin-5-yl)urea and (S)-1-(1-(4,6-difluoro-1-methyl-1H-benzo[d]imidazol-2-yl)-2,2,2-trifluoroethyl)-3-(2-(3-hydroxyazetidin-1-yl)pyrimidin-5-yl)urea

To a stirred mixture of 1-(2-chloropyrimidin-5-yl)-3-(1-(4,6-difluoro-1-methyl-1H-benzo[d]imidazol-2-yl)-2,2,2-trifluoroethyl)urea (85 mg, 0.20 mmol) and DIEA (0.11 mL, 0.61 mmol) in NMP (3 mL) was added azetidin-3-ol (74 mg, 1.0 mmol) at 25° C. under a nitrogen atmosphere. The resulting mixture was stirred for 16 hours at 80° C. under nitrogen. The product was purified by preparative HPLC (Column: XBridge Prep OBD C18 Column, 30*150 mm, 10 μm; mobile phase A: water (10 mM NH₄HCO₃+0.05% NH₄OH), mobile phase B: acetonitrile; flow rate: 60 mL/min; gradient: 24% B to 34% B in 8 min; wavelength: 254/220 nm; RT (min): 9.63) to afford 1-(1-(4,6-difluoro-1-methyl-1H-benzo[d]imidazol-2-yl)-2,2,2-trifluoroethyl)-3-(2-(3-hydroxyazetidin-1-yl)pyrimidin-5-yl)urea (50 mg, 0.11 mmol) as a white solid. LCMS RT 1.132 min, [M+H]j 458.10, LCMS method F.

1-(1-(4,6-difluoro-1-methyl-1H-benzo[d]imidazol-2-yl)-2,2,2-trifluoroethyl)-3-(2-(3-hydroxyazetidin-1-yl)pyrimidin-5-yl)urea (50 mg) was further purified by preparative chiral HPLC (column: CHIRAL ART Cellulose-SZ, 2.0*25 cm, 5 μm; mobile phase A: hexane (0.5% 2M NH₃ in MeOH), mobile phase B: EtOH; flow rate: 20 mL/min; gradient: 50% B isocratic; wavelength: 196/200 nm; RT1 (min): 4.5; RT2 (min): 6.6; sample solvent: MeOH:DCM 1:2; injection volume: 0.5 mL) to give (R)-1-(1-(4,6-difluoro-1-methyl-1H-benzo[d]imidazol-2-yl)-2,2,2-trifluoroethyl)-3-(2-(3-hydroxyazetidin-1-yl)pyrimidin-5-yl)urea and (S)-1-(1-(4,6-difluoro-1-methyl-1H-benzo[d]imidazol-2-yl)-2,2,2-trifluoroethyl)-3-(2-(3-hydroxyazetidin-1-yl)pyrimidin-5-yl)urea, both as a white solid.

Isomer 1: 7 mg, LCMS RT 1.167 min, [M+H]⁺ 458.15, LCMS method F. ¹H NMR (300 MHz, DMSO-d6) δ 8.58 (s, 1H), 8.37 (s, 2H), 7.74 (d, J=9.1 Hz, 1H), 7.50 (dd, J=8.9, 2.3 Hz, 1H), 7.19 (td, J=10.6, 2.2 Hz, 1H), 6.28-6.18 (m, 1H), 5.65 (d, J=6.5 Hz, 1H), 4.59-4.49 (m, 1H), 4.18 (dd, J=9.1, 6.6 Hz, 2H), 3.90 (s, 3H), 3.80-3.71 (m, 2H).

Isomer 2: 5 mg, LCMS RT 1.180 min, [M+H]⁺ 458.10, LCMS method F. ¹H NMR (300 MHz, DMSO-d6) δ 8.58 (s, 1H), 8.37 (s, 2H), 7.74 (d, J=9.1 Hz, 1H), 7.50 (dd, J=8.7, 2.2 Hz, 1H), 7.19 (td, J=10.6, 2.2 Hz, 1H), 6.27-6.15 (m, 1H), 5.65 (d, J=6.5 Hz, 1H), 4.60-4.45 (m, 1H), 4.18 (dd, J=9.1, 6.6 Hz, 2H), 3.90 (s, 3H), 3.73 (dd, J=9.1, 4.6 Hz, 2H).

Additional compounds prepared according to the methods of Example 51 are listed in Table 13 below. Corresponding ¹H NMR and mass spectrometry characterization for these compounds are described in Table 1. Certain compounds in Table 13 below were prepared with other compounds whose preparation is described further below in the Examples.

TABLE 13
Additional exemplary compounds

I-3676

### Example 52

### (R)-1-(2-(azetidin-1-yl)pyrimidin-5-yl)-3-(1-(3-chloro-2,6-difluorophenyl)-2,2,2-trifluoroethyl)urea

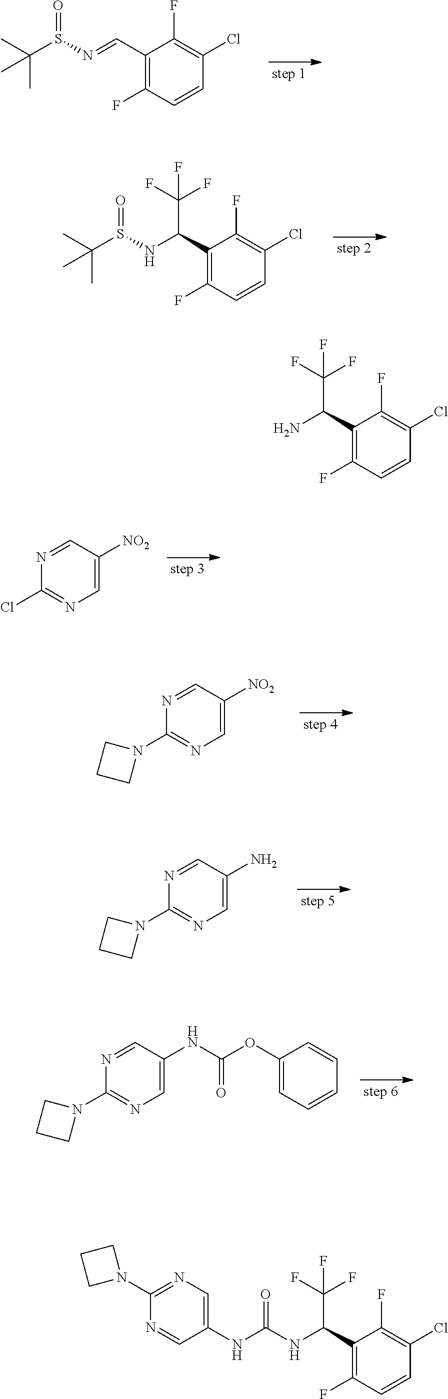

### Step 1. Synthesis of (S)—N—((R)-1-(3-chloro-2,6-difluorophenyl)-2,2,2-trifluoroethyl)-2-methylpropane-2-sulfinamide

A solution of (S,E)-N-(3-chloro-2,6-difluorobenzylidene)-2-methylpropane-2-sulfinamide (1.96 g, 7 mmol) and tetrabutylammoniumdifluorotriphenylsilicate (4.86 g, 9 mmol) in THF (15 mL) was stirred for 1 hour at −60° C. under a nitrogen atmosphere. Trifluoromethyltrimethylsilane (1.14 g, 8 mmol) was added at −60° C. The resulting mixture was stirred at −60° C. for 1 hour. After warming to room temperature water was added, and the solution was extracted with EtOAc (3×100 mL). The combined organic layers were washed with brine (3×50 mL) and dried over anhydrous Na₂SO₄. After filtration, the filtrate was concentrated under reduced pressure. The residue was purified by reverse phase flash chromatography (column, C18 gel; mobile phase, acetonitrile in water (0.1% NH₄OH), gradient: 10% to 90% acetonitrile in 40 min; detector: UV 254 nm) to give (S)—N—((R)-1-(3-chloro-2,6-difluorophenyl)-2,2,2-trifluoroethyl)-2-methylpropane-2-sulfinamide (600 mg, 1.6 mmol) as a white solid. LCMS RT 1.390 min, [M+H]⁺ 350, LCMS method E.

### Step 2. Synthesis of (R)-1-(3-chloro-2,6-difluorophenyl)-2,2,2-trifluoroethan-1-amine

To a stirred solution of (S)—N—((R)-1-(3-chloro-2,6-difluorophenyl)-2,2,2-trifluoroethyl)-2-methylpropane-2-sulfinamide (600 mg, 1.72 mmol) in 1,4-dioxane (10 mL) was added HCl (8.58 mL, 2 M in MeOH, 17.2 mmol) dropwise at room temperature under a nitrogen atmosphere. The resulting mixture was stirred for 2 hours at 30° C. under nitrogen. The solution was concentrated under reduced pressure. The residue was purified by trituration with Et₂O (3×5 mL). The crude product (R)-1-(3-chloro-2,6-difluorophenyl)-2,2,2-trifluoroethan-1-amine (400 mg, 1.5 mmol) was used in the next step directly without further purification. LCMS RT 1.390 min, [M+H]⁺ 246, LCMS method E.

### Step 3. Synthesis of 2-(azetidin-1-yl)-5-nitropyrimidine

To a stirred solution of 2-chloro-5-nitropyrimidine (0.96 g, 6 mmol) and azetidine (0.46 g, 8 mmol) in DMF (5 mL) was added K₂CO₃ (2.76 g, 0.02 mol) at room temperature under a nitrogen atmosphere. The resulting mixture was stirred for 1 hour at 90° C. under nitrogen. The mixture was allowed to cool down to room temperature and diluted with water. The solution was extracted with EtOAc (3×60 mL). The combined organic layers were washed with brine (5×10 mL) and dried over anhydrous Na₂SO₄. After filtration, the filtrate was concentrated under reduced pressure. The residue was purified by silica gel column chromatography, eluting with petroleum ether/EtOAc (1:1) to afford 2-(azetidin-1-yl)-5-nitropyrimidine (430 mg, 2.39 mmol) as a white solid. LCMS RT 0.360 min, [M+H]f 181, LCMS method E.

### Step 4. Synthesis of 2-(azetidin-1-yl)pyrimidin-5-amine

To a stirred solution of 2-(azetidin-1-yl)-5-nitropyrimidine (430 mg, 2.39 mmol) in THF (8 mL) at room temperature was Pd/C (203 mg) added. The flask was purged with hydrogen and stirred for 12 hours under a hydrogen atmosphere. After filtration, the filtrate was concentrated under reduced pressure to afford 2-(azetidin-1-yl)pyrimidin-5-amine (220 mg, 1.46 mmol) as a white solid. LCMS RT 1.076 min, [M+H]⁺ 150.19. LCMS method E.

### Step 5. Synthesis of phenyl (2-(azetidin-1-yl)pyrimidin-5-yl)carbamate

To a stirred solution of 2-(azetidin-1-yl)pyrimidin-5-amine (60 mg, 0.40 mmol) and phenyl carbonochloridate (63 mg, 0.40 mmol) in DMF (2 mL) was added DIEA (0.21 mL, 1.2 mmol) dropwise at room temperature under a nitrogen atmosphere. The resulting mixture was stirred for 1 hour at 0° C. under nitrogen. The crude product was used in the next step directly without purification. LCMS RT 0.755 min, [M+H]⁺ 271, LCMS method E.

### Step 6. Synthesis of (R)-1-(2-(azetidin-1-yl)pyrimidin-5-yl)-3-(1-(3-chloro-2,6-difluorophenyl)-2,2,2-trifluoroethyl)urea

To a stirred solution of phenyl (2-(azetidin-1-yl)pyrimidin-5-yl)carbamate (50 mg, 0.18 mmol) and (R)-1-(3-chloro-2,6-difluorophenyl)-2,2,2-trifluoroethan-1-amine (45 mg, 0.18 mmol) in DMF (1 mL) was added DIEA (72 mg, 0.55 mmol) dropwise at room temperature under a nitrogen atmosphere. The resulting mixture was stirred for 3 hours at 30° C. under nitrogen. The mixture was allowed to cool down to room temperature. The resulting mixture was purified by reverse phase flash chromatography (Column: XBridge Prep OBD C18 Column, 30*150 mm, 10 μm; mobile phase A: water (10 mM NH₄HCO₃+0.05% NH₄OH), mobile phase B: acetonitrile; flow rate: 60 mL/min; gradient: 34% B to 49% B in 8 min; wavelength: 254/220 nm; RT (min): 9.32) to give (R)-1-(2-(azetidin-1-yl)pyrimidin-5-yl)-3-(1-(3-chloro-2,6-difluorophenyl)-2,2,2-trifluoroethyl)urea (4.5 mg, 11 μmol) as a white solid. LCMS RT 1.435 min, [M+H]⁺ 422.05, LCMS method F. ¹H NMR (300 MHz, DMSO-d6) δ 8.67 (s, 1H), 8.36 (s, 2H), 7.84 (td, J=8.8, 5.6 Hz, IH), 7.60 (d, J=9.9 Hz, 1H), 7.39 (t, J=9.2 Hz, 1H), 6.09 (p, J=9.0 Hz, 1H), 3.99 (t, J=7.4 Hz, 4H), 2.28 (p, J=7.5 Hz, 2H).

### Example 43

Selected compounds of the present disclosure were tested in an ADP-Glo Biochemical PIK3CA Kinase Assay. Compounds to be assayed were plated in 16 doses of 1:2 serial dilutions (20 nL volume each well) on a 1536-well plate, and the plate warmed to room temperature. PIK3CA enzyme (e.g., H1047R, E542K, E545K, or wild-type) (1 μL of 2 nM solution in Enzyme Assay Buffer (comprising 50 mM HEPES pH 7.4, 50 nM NaCl, 6 mM MgCl₂, 5 mM DTT and 0.03% CHAPS)) was added and shaken for 10 seconds and preincubated for 30 minutes. To the well was added 1 μL of 200 μM ATP and 20 μM of diC8-PIP2 in Substrate Assay Buffer (50 mM HEPES pH7.4, 50 mM NaCl, 5 mM DTT and 0.03% CHAPS) to start the reaction, and the plate was shaken for 10 seconds, then spun briefly at 1500 rpm, and then incubated for 60 minutes at room temperature. The reaction was stopped by adding 2 μL. of ADP-Glo reagent (Promega), and spinning briefly at 1500 rpm, and then incubating for 40 minutes. ADP-Glo Detection reagent (Promega) was added and the plate spun briefly at 1500 rpm, then incubated for 30 minutes. The plate was read on an Envision 2105 (Perkin Elmer), and the IC₅₀ values were calculated using Genedata software.

Results of the ADP-Glo Biochemical PIK3CA Kinase Assay using H1047R PIK3CA enzyme are presented in Table 1. Compounds having an IC₅₀ less than or equal to 100 nM are represented as “A”; compounds having an IC₅₀ greater than 100 nM but less than or equal to 500 nM are represented as “B”; compounds having an IC₅₀ greater than 500 nM but less than or equal to 1 μM are represented as “C”; compounds having an IC₅₀ greater than 1 μM but less than or equal to 10 μM are represented as “D”; and compounds having an IC₅₀ greater than 10 μM but less than or equal to 100 μM are represented as “E”.

### Example 44

Selected compounds of the present disclosure were tested in a MCF10A Cell-Based PIK3CA Kinase Assay, namely the CisBio Phospho-AKT (Ser473) HTRF assay, to measure the degree of PIK3CA-mediated AKT phosphorylation. MCF10A cells (immortalized non-transformed breast cell line) overexpressing hotspot PIK3CA mutations (including H1047R, E542K, and E545K mutations) were used. Cells were seeded at 5,000 cells per well in DMEM/F12 (Thermo Fisher Scientific) supplemented with 0.5 mg/mL hydrocortisone, 100 ng/mL Cholera Toxin, 10 μg/mL insulin, and 0.5% horse serum. Once plated, cells were placed in a 5% CO₂, 37° C. incubator to adhere overnight.

The following day, compounds were added to the cell plates in 12 doses of 1:3 serial dilutions. The dose response curves were run in duplicate. Compound addition was carried out utilizing an Echo 55 Liquid Handler acoustic dispenser (Labcyte). The cell plates were incubated for 2 hours in a 5% CO₂, 37° C. incubator. Following compound incubation, the cells were lysed for 60 min at room temperature. Finally, a 4-hour incubation with the HTRF antibodies was performed at room temperature. All reagents, both lysis buffer and antibodies, were used from the CisBio pAKT S473 HTRF assay kit, as per the manufacturers protocol. Plates were read on an Envision 2105 (Perkin Elmer), and the IC₅₀ values were calculated using Genedata software.

Results of the MCF10A Cell-Based PIK3CA Kinase Assay are presented in Table 1. Compounds having an IC₅₀ less than or equal to 1 μM are represented as “A”; compounds having an IC₅₀ greater than 1 μM but less than or equal to 5 μM are represented as “B”; compounds having an IC₅₀ greater than 5 μM but less than or equal to 10 μM are represented as “C”; compounds having an IC₅₀ greater than 10 μM but less than or equal to 36 μM are represented as “D”; and compounds having an IC₅₀ greater than 36 μM but less than or equal to 100 μM are represented as “E”.

### INCORPORATION BY REFERENCE

All publications and patents mentioned herein are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the present disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

LENGTHY TABLES
The patent application contains a lengthy table section. A copy of the table is available in electronic form from the USPTO web site (https://seqdata.uspto.gov/?pageRequest=docDetail&DocID=US20250313524A1). An electronic copy of the table will also be available from the USPTO upon request and payment of the fee set forth in 37 CFR 1.19(b)(3).

What is claimed is:

1. A compound of formula I: 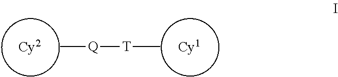
or a pharmaceutically acceptable salt thereof, wherein:
Cy¹ is phenyl; naphthyl; cubanyl; adamantyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; wherein Cy¹ is substituted with n instances of R¹;
Cy² is phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein Cy² is substituted with m instances of R²;
Q is L^{Q};
T is a bivalent C₁₋₃ aliphatic chain substituted with q instances of R^{T};
each R¹ is independently -L¹-R^{1A};
each R² is independently -L²-R^{2A};
each R¹ is independently -L¹-R^{TA}; or
two instances of R^{T} are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p¹ instances of R^{TTC};
two instances of R¹ are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p² instances of R^{11C};
two instances of R² are taken together with their intervening atoms to form a 3-7 membered saturated, partially unsaturated, or aromatic carbocyclic ring or a 3-7 membered saturated or partially unsaturated monocyclic heterocyclic ring having 1-3 heteroatoms independently selected from nitrogen, oxygen, and sulfur; wherein the ring is substituted with p³ instances of R^{22C};
one instance of R^{T} and one instance of R¹ are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p⁴ instances of R^{TLC}; or
one instance of R^{T} and one instance of R^{L} are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p⁵ instances of R^{TLC};
each of L¹, L², L^{Q}, and L^{T} is independently a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —N(R)C(NR)—, —N(R)C(NOR)—, —N(R)C(NCN)—, —C(O)N(R)—, —N(R)S(O)₂—, —S(O)₂N(R)—, —O—, —C(O)—, —OC(O)—, —C(O)O—, —S—, —S(O)—, or —S(O)₂—;
each R^{1A} is independently R^{A} or R^{B} substituted by r¹ instances of R^{1C};
each R^{2A} is independently R^{A} or R^{B} substituted by r² instances of R^{2C};
each R^{TA} is independently R^{A} or R^{B} substituted by r³ instances of R^{TC};
each R^{L} is independently R^{A} or R^{B} substituted by r⁴ instances of R^{LC};
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{1C}, R^{2C}, R^{TC}, R^{TTC}, R^{11C}, R^{22C}, R^{T1C}, R^{TLC}, and R^{LC} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of n, m, q, p¹, p², p³, p⁴, p⁵, r¹, r², r³, and r⁴ is independently 0, 1, 2, 3, 4, or 5.

2. The compound of claim 1, wherein Cy¹ is

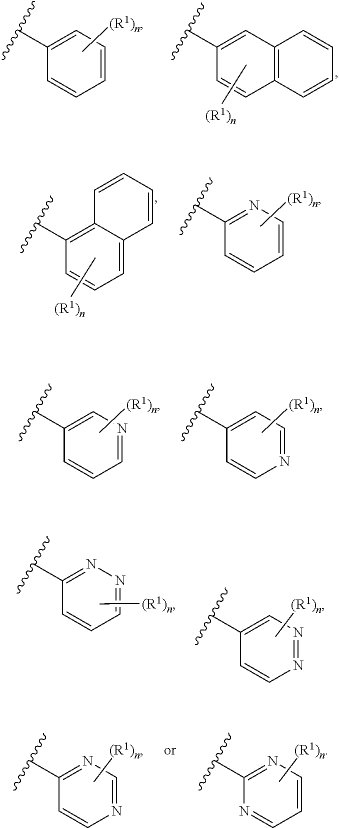

3. The compound of claim 1 or 2, wherein the compound is a compound of formula II: 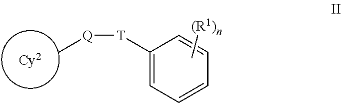
or a pharmaceutically acceptable salt thereof.

4. The compound of any one of the preceding claims, wherein Q is —C(O)N(R)—, —C(O)N(R)CH₂—, —N(R)—, —CH₂C(O)N(R)—, —N(R)C(O)N(R)—, or a covalent bond.

5. The compound of any one of the preceding claims, wherein T is 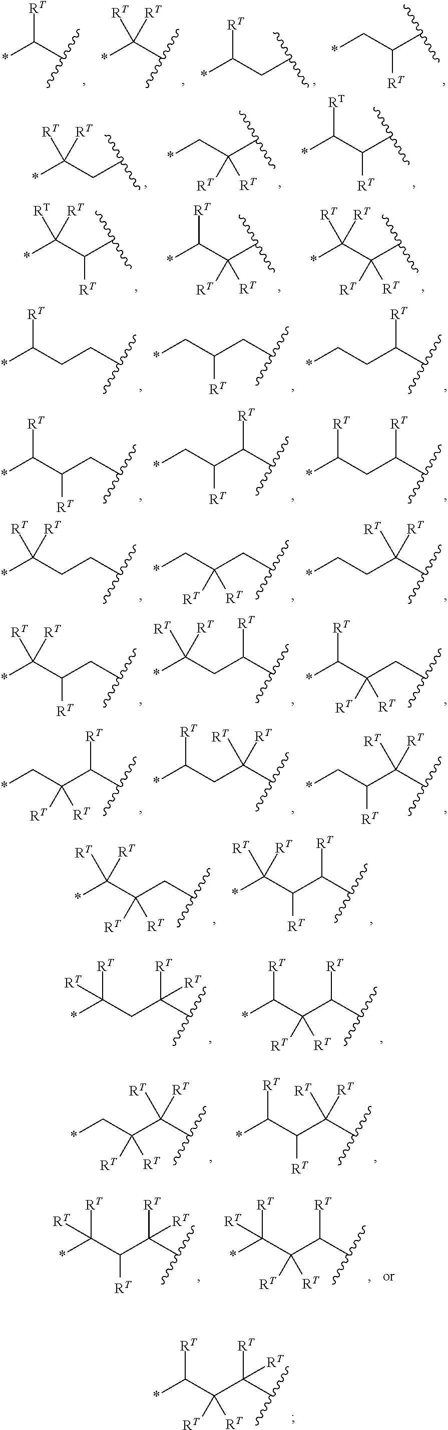
wherein [symbol] represents a bond to Q and [symbol] represents a bond to Cy¹.

6. The compound of any one of the preceding claims, wherein the compound is a compound of formula V, VI, VII, VIII, IX, or X: 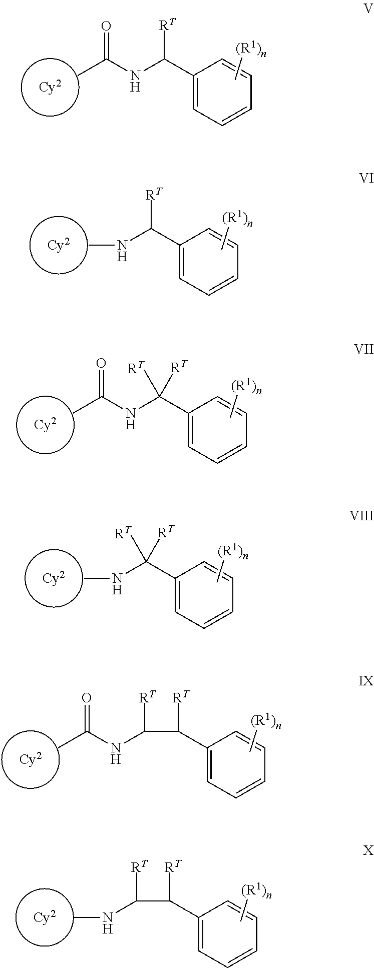
or a pharmaceutically acceptable salt thereof.

7. The compound of any one of the preceding claims, wherein two instances of R^{T} are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p¹ instances of R^{TTC}.

8. The compound of any one of claims 1 to 5, wherein T is 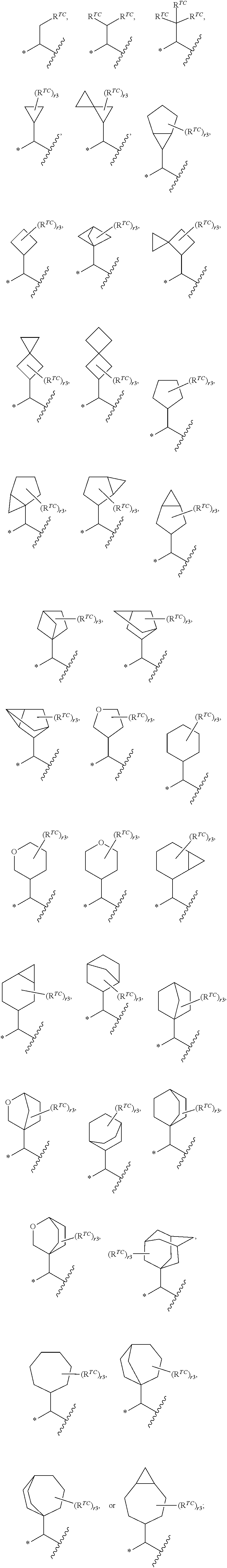
wherein [symbol] represents a bond to Q and [symbol] represents a bond to Cy¹.

9. The compound of any one of claims 1 to 6 and claim 8, wherein the compound is a compound of formula XI, XII, XIII, XIV, XV, XVI, XVII, XVIII, or XIX: 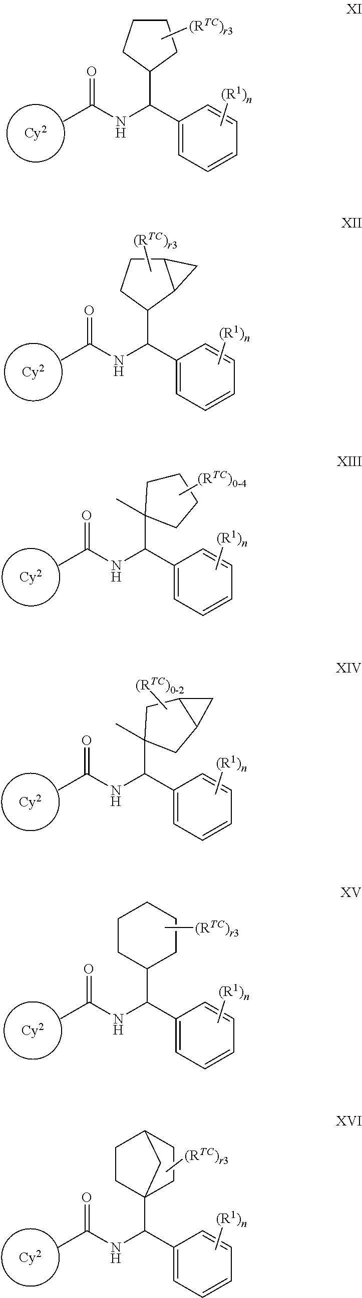 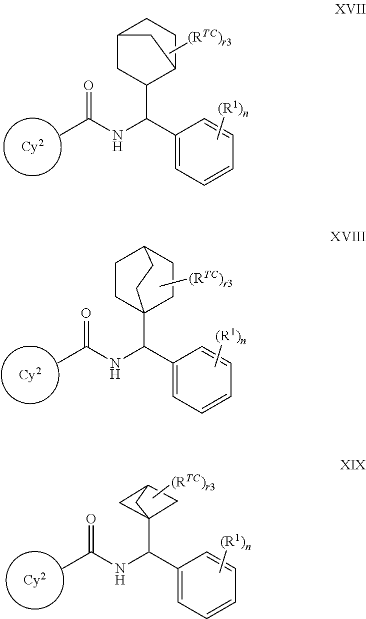
or a pharmaceutically acceptable salt thereof.

10. The compound of any one of claims 1-3, wherein the compound is a compound of formula XXVI, XXVII, XXVIII, XXIX, XXX, XXXI, XXXII, XXXIII, XXXIV, XXXV, or XXXVI: 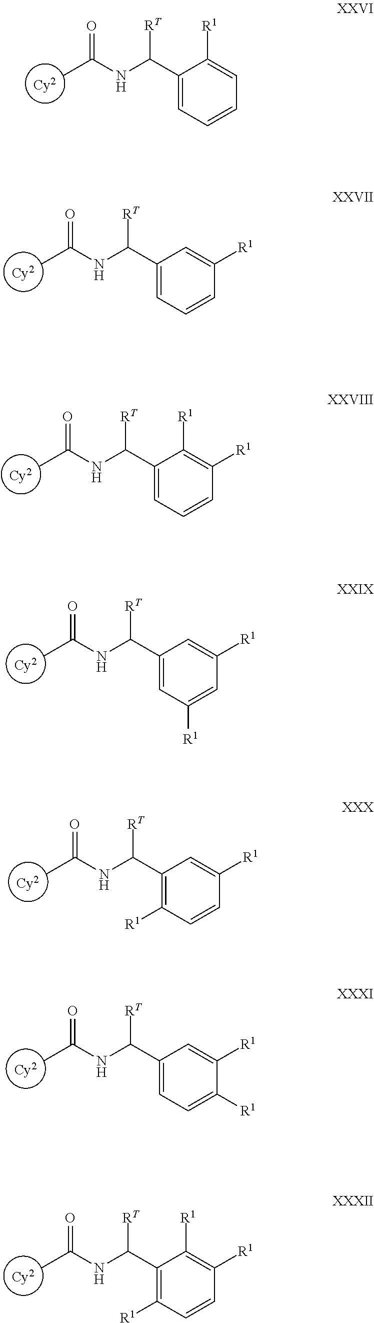 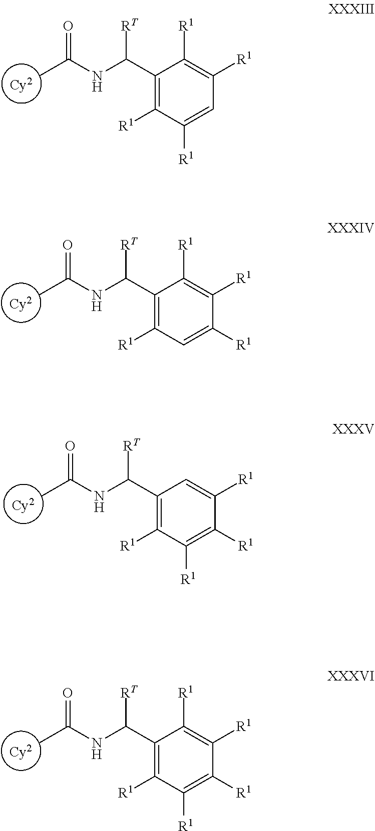
or a pharmaceutically acceptable salt thereof.

11. The compound of any one of the preceding claims, wherein Cy² is

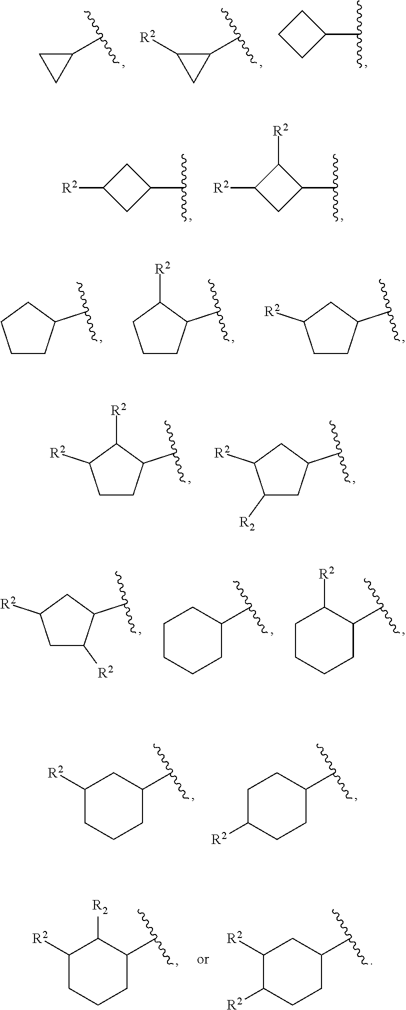

12. The compound of any one of claims 1 to 10, wherein Cy² is

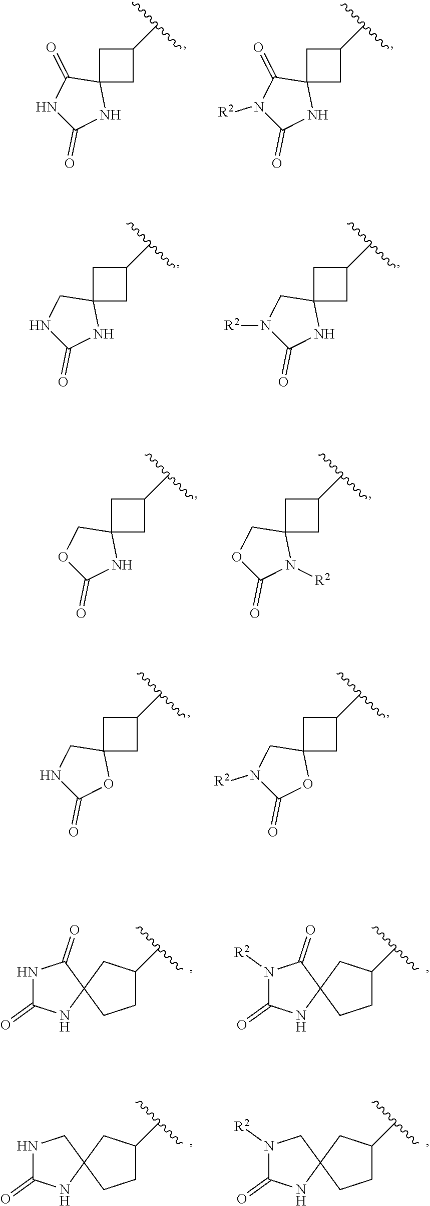

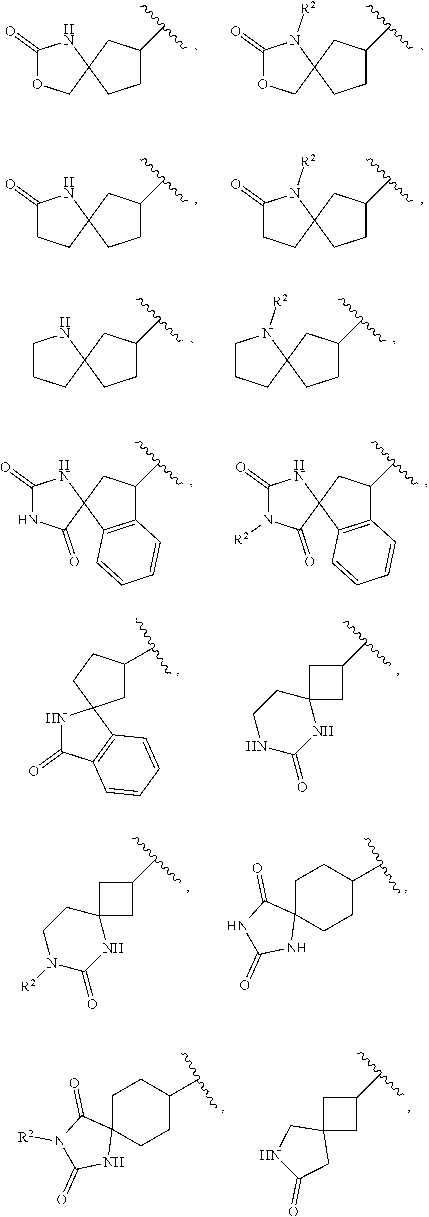

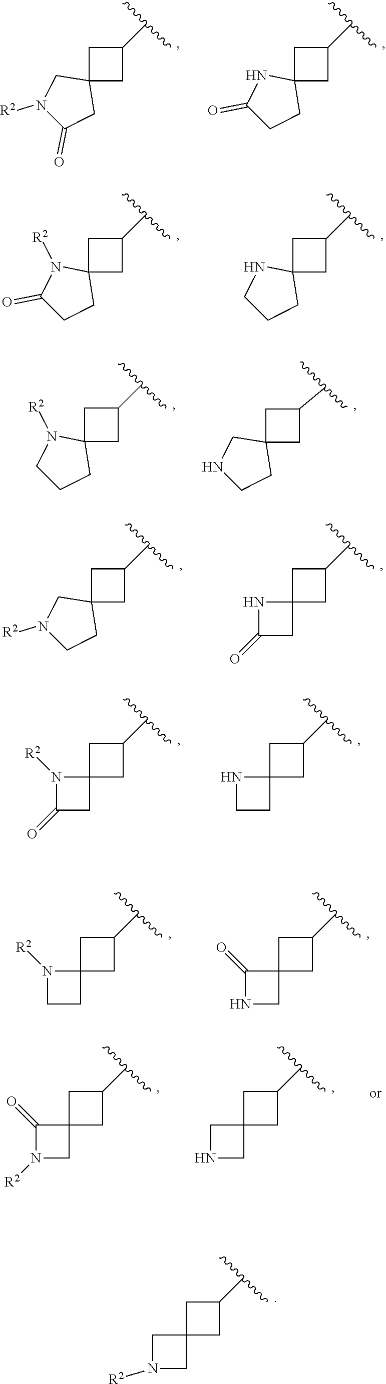

13. The compound of any one of claims 1-6, wherein the compound is a compound of formula XX, XXI, XXII, XXIII, XXIV, or XXV: 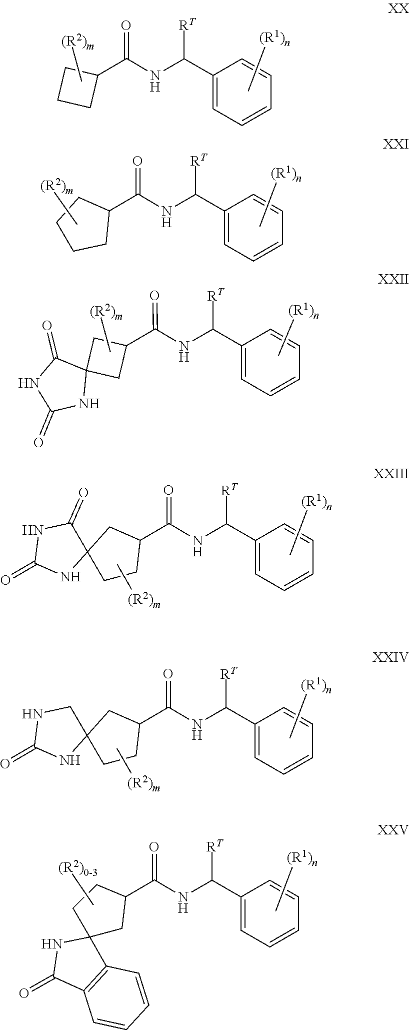
or a pharmaceutically acceptable salt thereof.

14. The compound of any one of the claims 1-6 and 10-13, wherein R^{T} is a 3-7 membered saturated or partially unsaturated monocyclic carbocyclic ring, or a 5-12 membered saturated or partially unsaturated bicyclic carbocyclic ring; each of which is substituted by r³ instances of R^{TC}.

15. The compound of any one of the preceding claims, wherein L¹ is a covalent bond.

16. The compound of any one of the preceding claims, wherein each R¹ is independently halogen, —CN, —O—(C₁₋₆ aliphatic chain substituted with 0-5 halogens), or a C₁₋₆ aliphatic chain substituted with 0-5 halogens.

17. The compound of any one of the preceding claims, wherein at least one R¹ is halogen.

18. The compound of any one of the preceding claims, wherein at least two R¹ are halogen.

19. The compound of any one of the preceding claims, wherein at least three R¹ are halogen.

20. The compound of any one of the preceding claims, wherein at least one R¹ is C₁₋₃ aliphatic optionally substituted with 1-3 halogen.

21. The compound of any one of the preceding claims, wherein at least one R¹ is —O—C₁₋₃ aliphatic optionally substituted with 1-3 halogen.

22. The compound of any one of the preceding claims, wherein n is 1, 2, 3, 4, or 5.

23. The compound of any one of the preceding claims, wherein R² is —N(R)—R^{2A}, —N(R)C(O)—R^{2A}, —CH(R^{L})N(R)—R^{2A}, —N(R)C(O)CH(R^{L})—R^{2A}, —CH(R^{L})O—R^{2A}, —CH(R^{L})—R^{2A}, or —R^{2A}.

24. The compound of any one of the preceding claims, wherein R² is —N(H)—R^{2A}, —N(H)C(O)—R^{2A}, or —CH₂—R^{2A}.

25. The compound of any one of the preceding claims, wherein R^{2A} is R^{B} substituted by r² instances of R^{2C}.

26. The compound of any one of the preceding claims, wherein R^{2A} is phenyl; naphthyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{2A} is substituted by r² instances of R^{2C}.

27. The compound of any one of the preceding claims, wherein R^{2A} is phenyl substituted with r² instances of R^{2C}.

28. The compound of any one of claims 1-25, wherein R^{2A} is a 5-6 membered monocyclic heteroaryl ring having 1-4 heteroatoms independently selected from nitrogen, oxygen, and sulfur; wherein said ring is substituted by r² instances of R^{2C}.

29. The compound of any one of claims 1-25, wherein R^{2A} is a 3-7 membered saturated or partially unsaturated monocyclic heterocyclic ring having 1-2 heteroatoms independently selected from nitrogen, oxygen, and sulfur; wherein said ring is substituted by r² instances of R^{2C}.

30. The compound of any one of claims 1-25, wherein R² is

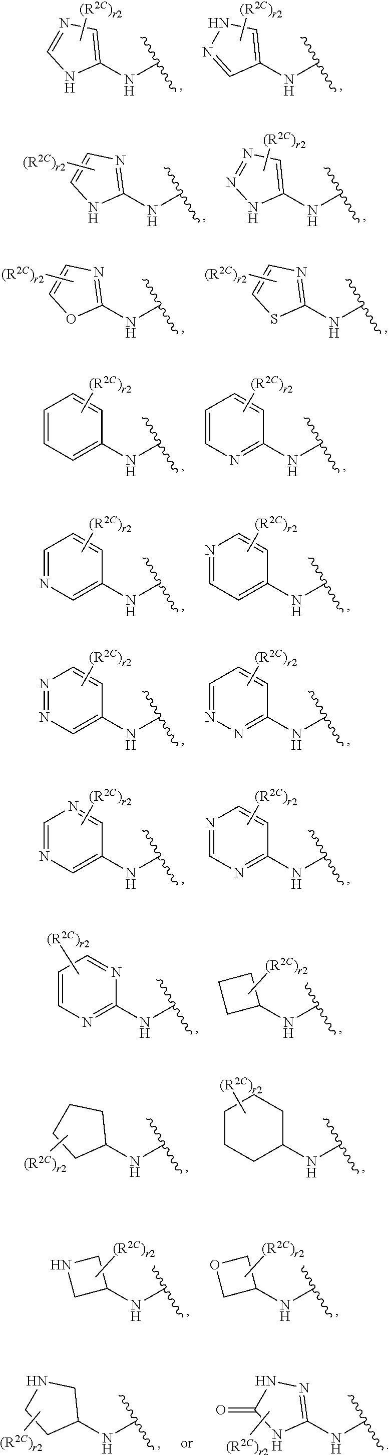

31. The compound of any one of claims 1-25, wherein R² is

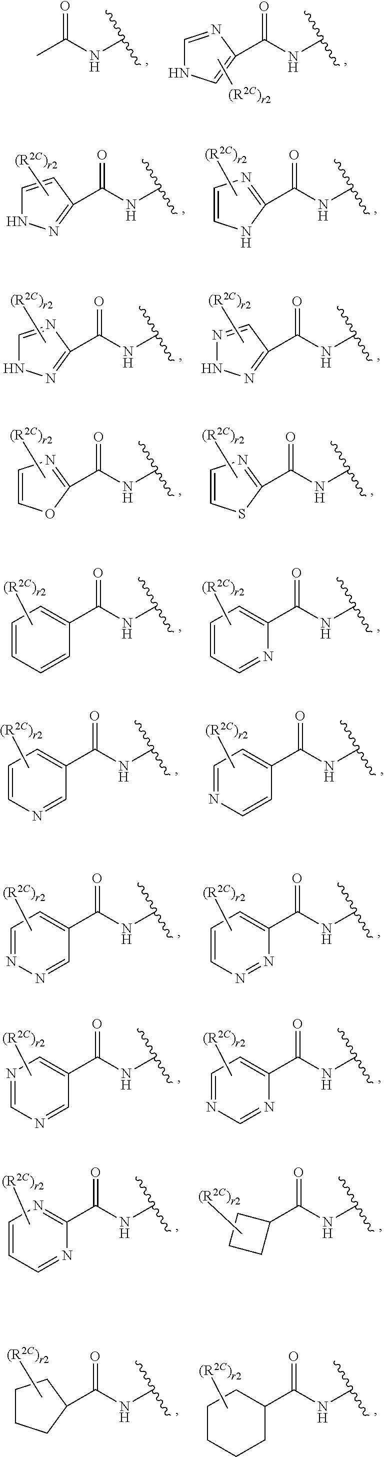

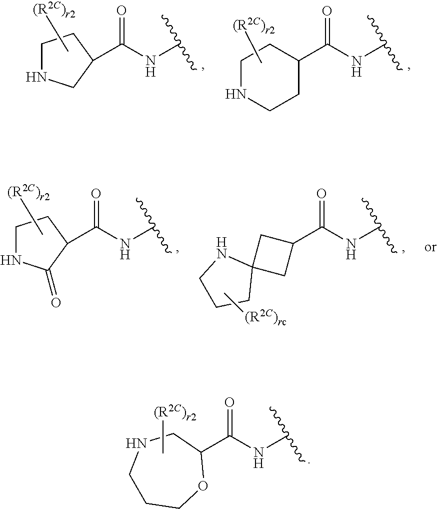

32. The compound of any one of claims 1-25, wherein R² is

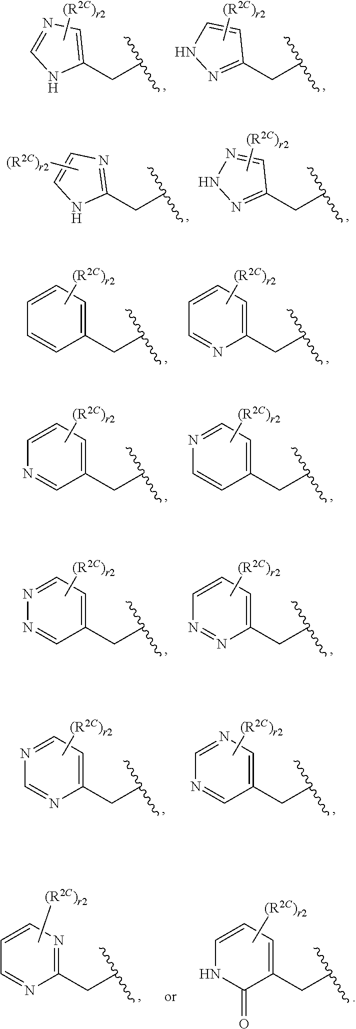

33. The compound of any one of the preceeding claims, wherein m is 1.

34. The compound of any one of claims 1-33, wherein each instance of R^{2C} is independently a C₁₋₆ aliphatic optionally substituted with (i) 1 or 2 groups independently selected from —O—(C₁₋₆ aliphatic), —OH, —N(C₁₋₆ aliphatic)₂, and —CN, and (ii) 1, 2, or 3 atoms independently selected from halogen and deuterium.

35. The compound of any one of claims 1-33, wherein each instance of R^{2C} is independently oxo, deuterium, halogen, —CN, —OH, —O—(C₁₋₃ aliphatic), or C₁₋₃ aliphatic, wherein each C₁₋₃ aliphatic is optionally substituted with one or more halogen atoms.

36. The compound of any one of claims 1-35, wherein each instance of R^{TC} is independently oxo, deuterium, halogen, —CN, —OH, —O—(C₁₋₃ aliphatic), or C₁₋₃ aliphatic, wherein each C₁₋₃ aliphatic is optionally substituted with one or more halogen atoms.

37. A compound selected from those set forth in Table 1, or a pharmaceutically acceptable salt thereof.

38. A pharmaceutical composition, comprising a compound of any one of the preceding claims, and a pharmaceutically acceptable carrier.

39. A method of inhibiting PI3Kα signaling activity in a subject, comprising administering a therapeutically effective amount of a compound of any of claims 1-37, or the pharmaceutical composition of claim 38, to a subject in need thereof.

40. A method of treating a PI3Kα-mediated disorder in a subject, comprising administering a therapeutically effective amount of a compound of any of claims 1-37, or the pharmaceutical composition of claim 38, to a subject in need thereof.

41. A method of treating a cellular proliferative disease in a subject, comprising administering a therapeutically effective amount of a compound of any of claims 1-37, or the pharmaceutical composition of claim 38, to a subject in need thereof.

42. The method of claim 41, wherein the cellular proliferative disease is cancer.

43. The method of claim 42, wherein the cancer is breast cancer.

44. The method of claim 42, wherein the cancer is ovarian cancer.

45. The method of claim 44, wherein the ovarian cancer is clear cell ovarian cancer.

46. The method of any one of claims 39-45, wherein the subject has PI3Kα containing at least one of the following mutations: H1047R, E542K, and E545K.
